(19) United States
(12) Patent Application Publication
(10) Pub. No.: US 2025/0145665 A1
(43) Pub. Date: May 8, 2025

(54) MACROCYCLIC PEPTIDES USEFUL AS MODULATORS OF TNF ALPHA

(71) Applicant: Bristol-Myers Squibb Company, Princeton, NJ (US)

(72) Inventors: Yong Zhang, West Windsor, NJ (US); Alaric J. Dyckman, Lawrenceville, NJ (US); Ving G. Lee, Hamilton Township, NJ (US); Zheming Ruan, Dayton, NJ (US); Jason Goodrich, Bridgewater, NJ (US); Lidet A. Negash, Plainsboro, NJ (US); Saleem Ahmad, Wall, NJ (US); Hong Wu, New Hope, NJ (US); John Hynes, Washington Crossing, PA (US); Michael A. Poss, Lawrenceville, NJ (US); Alexander C. Brueckner, Noblesville, IN (US); Sirish Kaushik Lakkaraju, Pennington, NJ (US)

(21) Appl. No.: 18/789,771

(22) Filed: Jul. 31, 2024

Related U.S. Application Data
(60) Provisional application No. 63/517,153, filed on Aug. 2, 2023.

(51) Int. Cl. C07K 7/56; A61K 38/00; A61P 37/06

(52) U.S. Cl. CPC C07K 7/56; A61P 37/06; A61K 38/00

(57) ABSTRACT

Disclosed are macrocyclic peptide compounds or a salt thereof, which are useful as modulators of TNFα, and pharmaceutical compositions comprising such compounds. These compounds may be useful in treating cancers, inflammatory and autoimmune diseases.

### CROSS-REFERENCE TO RELATED APPLICATIONS

This application claims the benefit of U.S. Provisional Application No. 63/517,153, filed Aug. 2, 2023, the disclosure of which is incorporated herein by reference in its entirety.

### DESCRIPTION

The present invention generally relates to macrocyclic peptides useful as modulators of TNFα signaling. Provided herein are macrocyclic peptides, compositions comprising such compounds, and methods of their use. The invention further pertains to pharmaceutical compositions containing at least one compound according to the invention that are useful for the treatment of conditions related to TNFα activity, including inflammatory and autoimmune disorders.

TNFα is the first and archetypical member of the TNF superfamily (TNFSF) of ligands. TNFSF ligands are involved in the regulation of several key biological processes including cell differentiation, cell survival, cell death, and inflammation. Ligands of the TNF superfamily play a pivotal role in the regulation and orchestration of the immune and inflammatory responses at multiple levels. A common structural feature of TNFSF ligands is the formation of trimeric complexes that can bind to and activate specific TNFSF receptors. Similar to several other family members, TNFα is a type II transmembrane protein that can be secreted as a soluble form following proteolytic cleavage by a metalloprotease. Both the transmembrane and soluble forms of TNFα form biologically active trimeric complexes that signal through TNF receptors 1 and 2. TNFα can act on multiple cell types (T cells, monocytes, endothelial cells) through TNFRs to induce activation of the immune system, production of inflammatory cytokines, osteoclastogenesis, and cell death.

Based on their physiological and pathophysiological functions, TNF and TNFSF ligands are implicated in the pathogenesis of a number of inflammatory and autoimmune disorders (see, for example, E. C. Keystone et al., J Rheumatol, 2010, 37, 27-39; and L. M. Sedger & M. F. McDermott, Cytokine Growth Factor Rev, 2014, 25 (4), 453-72). To date, a number of TNFα modulating agents have been developed and are commercially available. The mechanism of action of clinically-proven protein-based therapeutic agents directed against TNFα is to act as competitive antagonists to inhibit TNFα from binding to TNFR1 and TNFR2. These agents include antibodies specific to TNFα including adalimumab, golimumab, certolizumab pegol, and infliximab. Another approved agent for the treatment of TNFα-mediated disorders is etanercept, a chimera of the immunoglobulin molecule and the TNFR2 ectodomain which also prevents TNFα from binding to the cellular receptors.

Being modulators of human TNFα activity, the macrocyclic peptides are beneficial in the treatment and/or prevention of a number of human maladies. These include inflammatory and autoimmune disorders, neurological and neurodegenerative disorders, pain and nociceptive disorders, cardiovascular disorders, metabolic disorders, ocular disorders, and oncological disorders.

WO 2013/186229, WO 2014/009295, and WO 2014/009296 disclose compounds useful as modulators of TNFα.

In view of the numerous conditions that are contemplated to benefit by treatment involving modulation of TNF, it is immediately apparent that new compounds capable of modulating the signaling of TNFα and methods of using these compounds should provide substantial therapeutic benefits to a wide variety of patients.

The present invention relates to a new class of macrocyclic peptides found to be effective inhibitors of TNFα activity. These compounds are provided to be useful as pharmaceuticals with desirable stability, bioavailability, therapeutic index, and toxicity values that are important to their drugability.

### Definitions

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof. Embodiments identified herein as exemplary or preferred are intended to be illustrative and not limiting.

Unless specifically stated otherwise herein, references made in the singular may also include the plural. For example, “a” and “an” may refer to either one, or one or more.

As used herein, the phrase “compounds” refers to at least one compound. For example, a compound of Formula (I) includes a compound of Formula (I); and two or more compounds of Formula (I).

Unless otherwise indicated, any heteroatom with unsatisfied valences is assumed to have hydrogen atoms sufficient to satisfy the valences.

The definitions set forth herein take precedence over definitions set forth in any patent, patent application, and/or patent application publication incorporated herein by reference.

Listed below are definitions of various terms used to describe the present invention. These definitions apply to the terms as they are used throughout the specification (unless they are otherwise limited in specific instances) either individually or as part of a larger group.

Throughout the specification, groups and substituents thereof may be chosen by one skilled in the field to provide stable moieties and compounds.

In accordance with a convention used in the art,
[symbol] is used in structural formulas herein to depict the bond that is the point of attachment of the moiety or substituent to the core or backbone structure.

The terms “halo” and “halogen,” as used herein, refer to F, Cl, Br, and I.

The term “cyano” refers to the group —CN.

The term “amino” refers to the group —NH₂.

The term “hydroxy” refers to the group —OH.

The term “nitro” refers to the group —NO₂.

The term “oxo” refers to the group ═O.

The term “alkyl” as used herein, refers to both branched and straight-chain saturated aliphatic hydrocarbon groups containing, for example, from 1 to 12 carbon atoms, from 1 to 6 carbon atoms, and from 1 to 4 carbon atoms. Examples of alkyl groups include, but are not limited to, methyl (Me), ethyl (Et), propyl (e.g., n-propyl and i-propyl), butyl (e.g., n-butyl, i-butyl, sec-butyl, and t-butyl), and pentyl (e.g., n-pentyl, isopentyl, neopentyl), n-hexyl, 2-methylpentyl, 2-ethylbutyl, 3-methylpentyl, and 4-methylpentyl. When numbers appear in a subscript after the symbol “C”, the subscript defines with more specificity the number of carbon atoms that a particular group may contain. For example, “C₁₋₆ alkyl” denotes straight and branched chain alkyl groups with one to six carbon atoms.

The term “haloalkyl” as used herein is intended to include both branched and straight-chain saturated aliphatic hydrocarbon groups substituted with one or more halogen atoms. For example, “C₁₋₄ haloalkyl” is intended to include C₁, C₂, C₃, and C₄ alkyl groups substituted with one or more halogen atoms. Representative examples of haloalkyl groups include, but are not limited to, —CF₃, —CCl₃, —CFCl₂, and —CH₂CF₃.

The term “fluoroalkyl” as used herein is intended to include both branched and straight-chain saturated aliphatic hydrocarbon groups substituted with one or more fluorine atoms. For example, “C₁₋₄ fluoroalkyl” is intended to include C₁, C₂, C₃, and C₄ alkyl groups substituted with one or more fluorine atoms. Representative examples of fluoroalkyl groups include, but are not limited to, —CF₃ and —CH₂CF₃.

The term “hydroxyalkyl” includes both branched and straight-chain saturated alkyl groups substituted with one or more hydroxyl groups. For example, “hydroxyalkyl” includes —CH₂OH, —CH₂CH₂OH, and C₁₋₄ hydroxyalkyl.

The term “cycloalkyl,” as used herein, refers to a group derived from a non-aromatic monocyclic or polycyclic hydrocarbon molecule by removal of one hydrogen atom from a saturated ring carbon atom. Representative examples of cycloalkyl groups include, but are not limited to, cyclopropyl, cyclopentyl, and cyclohexyl. When numbers appear in a subscript after the symbol “C”, the subscript defines with more specificity the number of carbon atoms that a particular cycloalkyl group may contain. For example, “C₃₋₆ cycloalkyl” denotes cycloalkyl groups with three to six carbon atoms.

The term “alkoxy,” as used herein, refers to an alkyl group attached to the parent molecular moiety through an oxygen atom, for example, methoxy group (—OCH₃). For example, “C₁₋₃ alkoxy” denotes alkoxy groups with one to three carbon atoms.

The terms “haloalkoxy” and “—O(haloalkyl)” represent a haloalkyl group as defined above attached through an oxygen linkage (—O—). For example, “C₁₋₄ haloalkoxy” is intended to include C₁, C₂, C₃, and C₄ haloalkoxy groups.

The terms “fluoroalkoxy” and “—O(fluoroalkyl)” represent a fluoroalkyl group as defined above attached through an oxygen linkage (—O—). For example, “C₁₋₄ fluoroalkoxy” is intended to include C₁, C₂, C₃, and C₄ fluoroalkoxy groups.

The terms “carbocyclo”, “carbocyclic” or “carbocyclyl” may be used interchangeably and refer to cyclic groups having at least one saturated or partially saturated non-aromatic ring wherein all atoms of all rings are carbon. The carbocyclyl ring may be unsubstituted or may contain one or more substituents as valence allows. Thus, the term includes nonaromatic rings such as for example, cycloalkyl, cycloalkenyl, and cycloalkynyl rings. Exemplary bicyclic carbocyclyl groups include, indanyl, indenyl, dihydronaphthalenyl, tetrahydronaphthenyl, hexahydronaphthalenyl, octahydronaphthalenyl, decahydronaphthalenyl, bicycloheptanyl, bicyclooctanyl, and bicyclononanyl.

The term “aryl” as used herein, refers to a group of atoms derived from a molecule containing aromatic ring(s) by removing one hydrogen that is bonded to the aromatic ring(s). Heteroaryl groups that have two or more rings must include only aromatic rings. Representative examples of aryl groups include, but are not limited to, phenyl and naphthyl. The aryl ring may be unsubstituted or may contain one or more substituents as valence allows.

The term “benzyl,” as used herein, refers to a methyl group in which one of the hydrogen atoms is replaced by a phenyl group. The phenyl ring may be unsubstituted or may contain one or more substituents as valence allows.

The term “heteroatom” refers to oxygen (O), sulfur(S), and nitrogen (N).

The terms “heterocyclo”, “heterocyclic”, or “heterocyclyl” may be used interchangeably and refer to cyclic groups having at least saturated or partially saturated non-aromatic ring and wherein one or more of the rings have at least one heteroatom (O, S or N), said heteroatom containing ring preferably having 1 to 3 heteroatoms independently selected from O, S, and/or N. The ring of such a group containing a heteroatom can contain one or two oxygen or sulfur atoms and/or from one to four nitrogen atoms provided that the total number of heteroatoms in each ring is four or less, and further provided that the ring contains at least one carbon atom. The nitrogen and sulfur atoms may optionally be oxidized and the nitrogen atoms may optionally be quaternized. The heterocyclo group may be attached at any available nitrogen or carbon atom. The heterocyclo ring may be unsubstituted or may contain one or more substituents as valence allows.

Exemplary monocyclic heterocyclyl groups include pyrrolidinyl, imidazolinyl, oxazolidinyl, isoxazolinyl, thiazolidinyl, isothiazolidinyl, tetrahydrofuranyl, piperidinyl, piperazinyl, 2-oxopiperazinyl, 2-oxopiperidinyl, 2-oxopyrrolodinyl, 2-oxoazepinyl, azepinyl, 4-piperidonyl, tetrahydropyranyl, morpholinyl, thiamorpholinyl, thiamorpholinyl sulfoxide, thiamorpholinyl sulfone, 1,3-dioxolane, tetrahydro-1,1-dioxothienyl, dihydroisoindolyl, and tetrahydroquinolinyl

The term “heteroaryl” refers to substituted and unsubstituted aromatic 5- or 6-membered monocyclic groups and 9- or 10-membered bicyclic groups that have at least one heteroatom (O, S or N) in at least one of the rings, said heteroatom-containing ring preferably having 1, 2, or 3 heteroatoms independently selected from O, S, and/or N. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic group are aromatic and may contain only carbon atoms. The nitrogen and sulfur atoms may optionally be oxidized and the nitrogen atoms may optionally be quaternized. Bicyclic heteroaryl groups must include only aromatic rings. The heteroaryl group may be attached at any available nitrogen or carbon atom of any ring. The heteroaryl ring system may be unsubstituted or may contain one or more substituents.

Exemplary monocyclic heteroaryl groups include pyrrolyl, pyrazolyl, pyrazolinyl, imidazolyl, oxazolyl, isoxazolyl, thiazolyl, thiadiazolyl, isothiazolyl, furanyl, thiophenyl, oxadiazolyl, pyridinyl, pyrazinyl, pyrimidinyl, pyridazinyl, and triazinyl.

Exemplary bicyclic heteroaryl groups include indolyl, benzothiazolyl, benzodioxolyl, benzoxazolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, and pyrrolopyridyl.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds of Formula (I) can be provided as amorphous solids or crystalline solids. Lyophilization can be employed to provide the compounds of Formula (I) as amorphous solids.

It should further be understood that solvates (e.g., hydrates) of the compounds of Formula (I) are also within the scope of the present invention. The term “solvate” means a physical association of a compound of Formula (I) with one or more solvent molecules, whether organic or inorganic. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, isopropanolates, acetonitrile solvates, and ethyl acetate solvates. Methods of solvation are known in the art.

Various forms of prodrugs are well known in the art and are described in:
a) The Practice of Medicinal Chemistry, Camille G. Wermuth et al., Ch 31, (Academic Press, 1996);
b) Design of Prodrugs, edited by H. Bundgaard, (Elsevier, 1985);
c) A Textbook of Drug Design and Development, P. Krogsgaard-Larson and H. Bundgaard, eds. Ch 5, pgs 113-191 (Harwood Academic Publishers, 1991); and
d) Hydrolysis in Drug and Prodrug Metabolism, Bernard Testa and Joachim M. Mayer, (Wiley-VCH, 2003).

In addition, compounds of Formula (I), subsequent to their preparation, can be isolated and purified to obtain a composition containing an amount by weight equal to or greater than 99% of a compound of Formula (I) (“substantially pure”), which is then used or formulated as described herein. Such “substantially pure” compounds of Formula (I) are also contemplated herein as part of the present invention.

“Stable compound” and “stable structure” are meant to indicate a compound that is sufficiently robust to survive isolation to a useful degree of purity from a reaction mixture, and formulation into an efficacious therapeutic agent. The present invention is intended to embody stable compounds.

“Therapeutically effective amount” is intended to include an amount of a compound of the present invention alone or an amount of the combination of compounds claimed or an amount of a compound of the present invention in combination with other active ingredients effective to act as an inhibitor to TNFα, or effective to treat or prevent autoimmune and/or inflammatory disease states, such as multiple sclerosis and rheumatoid arthritis.

As used herein, “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting its development; and/or (c) relieving the disease-state, i.e., causing regression of the disease state.

The compounds of the present invention are intended to include all isotopes of atoms occurring in the present compounds. Isotopes include those atoms having the same atomic number but different mass numbers. By way of general example and without limitation, isotopes of hydrogen include deuterium (D) and tritium (T). Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. For example, methyl (—CH₃) also includes deuterated methyl groups such as —CD3.

Compounds in accordance with Formula (I) can be administered by any means suitable for the condition to be treated, which can depend on the need for site-specific treatment or quantity of Formula (I) compound to be delivered.

Also embraced within this invention is a class of pharmaceutical compositions comprising a compound of Formula (I) and one or more non-toxic, pharmaceutically-acceptable carriers and/or diluents and/or adjuvants (collectively referred to herein as “carrier” materials) and, if desired, other active ingredients. The compounds of Formula (I) may be administered by any suitable route, preferably in the form of a pharmaceutical composition adapted to such a route, and in a dose effective for the treatment intended. The compounds and compositions of the present invention may, for example, be administered orally, mucosally, or parentally including intravascularly, intravenously, intraperitoneally, subcutaneously, intramuscularly, and intrasternally in dosage unit formulations containing conventional pharmaceutically acceptable carriers, adjuvants, and vehicles. For example, the pharmaceutical carrier may contain a mixture of mannitol or lactose and microcrystalline cellulose. The mixture may contain additional components such as a lubricating agent, e.g. magnesium stearate and a disintegrating agent such as crospovidone. The carrier mixture may be filled into a gelatin capsule or compressed as a tablet. The pharmaceutical composition may be administered as an oral dosage form or an infusion, for example.

For oral administration, the pharmaceutical composition may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 0.1 to 1000 mg, preferably from about 0.25 to 250 mg, and more preferably from about 0.5 to 100 mg. A suitable daily dose for a human or other mammal may vary widely depending on the condition of the patient and other factors, but, can be determined using routine methods.

Any pharmaceutical composition contemplated herein can, for example, be delivered orally via any acceptable and suitable oral preparations. Exemplary oral preparations, include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the invention can contain at least one agent selected from sweetening agents, flavoring agents, coloring agents, demulcents, antioxidants, and preserving agents.

A tablet can, for example, be prepared by admixing at least one compound of Formula (I) with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents, such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water soluble taste masking materials, include, but are not limited to, hydroxypropyl-methylcellulose and hydroxypropyl-cellulose. Exemplary time delay materials, include, but are not limited to, ethyl cellulose and cellulose acetate butyrate.

Hard gelatin capsules can, for example, be prepared by mixing at least one compound of Formula (I) with at least one inert solid diluent, such as, for example, calcium carbonate; calcium phosphate; and kaolin.

Soft gelatin capsules can, for example, be prepared by mixing at least one compound of Formula (I) with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of Formula (I) with at least one excipient suitable for the manufacture of an aqueous suspension. Exemplary excipients suitable for the manufacture of an aqueous suspension, include, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of Formula (I) in either a vegetable oil, such as, for example, arachis oil; olive oil; sesame oil; and coconut oil; or in mineral oil, such as, for example, liquid paraffin. An oily suspension can also contain at least one thickening agent, such as, for example, beeswax; hard paraffin; and cetyl alcohol. In order to provide a palatable oily suspension, at least one of the sweetening agents already described hereinabove, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of Formula (I) with at least one dispersing and/or wetting agent; at least one suspending agent; and/or at least one preservative. Suitable dispersing agents, wetting agents, and suspending agents are as already described above. Exemplary preservatives include, but are not limited to, for example, anti-oxidants, e.g., ascorbic acid. In addition, dispersible powders and granules can also contain at least one excipient, including, but not limited to, for example, sweetening agents; flavoring agents; and coloring agents.

An emulsion of at least one compound of Formula (I) thereof can, for example, be prepared as an oil-in-water emulsion. The oily phase of the emulsions comprising compounds of Formula (I) may be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase may comprise merely an emulsifier, it may comprise a mixture of at least one emulsifier with a fat or an oil or with both a fat and an oil. Suitable emulsifying agents include, but are not limited to, for example, naturally-occurring phosphatides, e.g., soy bean lecithin; esters or partial esters derived from fatty acids and hexitol anhydrides, such as, for example, sorbitan monooleate; and condensation products of partial esters with ethylene oxide, such as, for example, polyoxyethylene sorbitan monooleate. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

The compounds of Formula (I) can, for example, also be delivered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as, for example, water, Ringer's solution, and isotonic sodium chloride solution; sterile oil-in-water microemulsions; and aqueous or oleaginous suspensions.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

A sterile injectable oil-in-water microemulsion can, for example, be prepared by 1) dissolving at least one compound of Formula (I) in an oily phase, such as, for example, a mixture of soybean oil and lecithin; 2) combining the Formula (I) containing oil phase with a water and glycerol mixture; and 3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butane diol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid.

Pharmaceutically acceptable carriers, adjuvants, and vehicles that may be used in the pharmaceutical compositions of this invention include, but are not limited to, ion exchangers, alumina, aluminum stearate, lecithin, self-emulsifying drug delivery systems (SEDDS) such as d-alpha-tocopherol polyethyleneglycol 1000 succinate, surfactants used in pharmaceutical dosage forms such as Tweens, polyethoxylated castor oil such as CREMOPHOR surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutical compositions can be presented in a pack or dispenser device which can contain one or more unit dosage forms including the compound of Formula (I). The pack can, for example, comprise metal or plastic foil, such as a blister pack. The pack or dispenser device can be accompanied by instructions for administration.

The pharmaceutically active compounds of this invention can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

The amounts of compounds that are administered and the dosage regimen for treating a disease condition with the compounds and/or compositions of this invention depends on a variety of factors, including the age, weight, sex, the medical condition of the subject, the type of disease, the severity of the disease, the route and frequency of administration, and the particular compound employed. Thus, the dosage regimen may vary widely, but can be determined routinely using standard methods. A daily dose of about 0.001 to 100 mg/kg body weight, preferably between about 0.0025 and about 50 mg/kg body weight and most preferably between about 0.005 to 10 mg/kg body weight, may be appropriate. The daily dose can be administered in one to four doses per day. Other dosing schedules include one dose per week and one dose per two day cycle.

For therapeutic purposes, the active compounds of this invention are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

Pharmaceutical compositions of this invention comprise at least one compound of Formula (I) and optionally an additional agent selected from any pharmaceutically acceptable carrier, adjuvant, and vehicle. Alternate compositions of this invention comprise a compound of the Formula (I) described herein, or a prodrug thereof, and a pharmaceutically acceptable carrier, adjuvant, or vehicle.

The pharmaceutical compositions may contain other therapeutic agents and may be formulated, for example, by employing conventional solid or liquid vehicles or diluents, as well as pharmaceutical additives of a type appropriate to the mode of desired administration (e.g., excipients, binders, preservatives, stabilizers, flavors, etc.) according to techniques such as those well known in the art of pharmaceutical formulation.

### General Information:

### Peptide Synthesis

The macrocyclic peptides of the present disclosure can be produced by methods known in the art, such as they can be synthesized chemically, recombinantly in a cell free system, recombinantly within a cell or can be isolated from a biological source. Chemical synthesis of a macrocyclic peptide of the present disclosure can be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. A preferred method to synthesize the macrocyclic peptides and analogs thereof described herein is chemical synthesis using various solid-phase techniques such as those described in Chan, W. C. et al, eds., Fmoc Solid Phase Synthesis, Oxford University Press, Oxford (2000); Barany, G. et al, The Peptides: Analysis, Synthesis, Biology, Vol. 2: “Special Methods in Peptide Synthesis, Part A”, pp. 3-284, Gross, E. et al, eds., Academic Press, New York (1980); in Atherton, E., Sheppard, R. C. Solid Phase Peptide Synthesis: A Practical Approach, IRL Press, Oxford, England (1989); and in Stewart, J. M. Young, J. D. Solid-Phase Peptide Synthesis, 2nd Edition, Pierce Chemical Co., Rockford, IL (1984). The preferred strategy is based on the (9-fluorenylmethyloxycarbonyl) group (Fmoc) for temporary protection of the α-amino group, in combination with the tert-butyl group (tBu) for temporary protection of the amino acid side chains (see for example Atherton, E. et al, “The Fluorenylmethoxycarbonyl Amino Protecting Group”, in The Peptides: Analysis, Synthesis, Biology, Vol. 9: “Special Methods in Peptide Synthesis, Part C”, pp. 1-38, Undenfriend, S. et al, eds., Academic Press, San Diego (1987).

The peptides can be synthesized in a stepwise manner on an insoluble polymer support (also referred to as “resin”) starting from the C-terminus of the peptide. A synthesis is begun by appending the C-terminal amino acid of the peptide to the resin through formation of an amide or ester linkage. This allows the eventual release of the resulting peptide as a C-terminal amide or carboxylic acid, respectively.

The C-terminal amino acid and all other amino acids used in the synthesis are required to have their a-amino groups and side chain functionalities (if present) differentially protected such that the a-amino protecting group may be selectively removed during the synthesis. The coupling of an amino acid is performed by activation of its carboxyl group as an active ester and reaction thereof with the unblocked a-amino group of the N-terminal amino acid appended to the resin. The sequence of a-amino group deprotection and coupling is repeated until the entire peptide sequence is assembled. The peptide is then released from the resin with concomitant deprotection of the side chain functionalities, usually in the presence of appropriate scavengers to limit side reactions. The resulting peptide is finally purified by reverse phase HPLC LC-MS.

The syntheses of the peptide analogs described herein can be carried out by using a single or multi-channel peptide synthesizer, such as an CEM Liberty Microwave synthesizer, or a Protein Technologies, Inc. Prelude (6 channels) or Symphony (12 channels) or Symphony X (24 channels) synthesizer.

The peptidyl-resin precursors for their respective peptides may be cleaved and deprotected using any standard procedure (see, for example, King, D. S. et al, Int. J. Peptide Protein Res., 36:255-266 (1990)). A desired method is the use of TFA in the presence of TIS as scavenger and DTT or TCEP as the disulfide reducing agent. Typically, the peptidyl-resin is stirred in TFA/TIS/DTT (95:5:1 to 97:3:1), v:v:w; 1-3 mL/100 mg of peptidyl resin) for 1.5-3 h at rt. The spent resin is then filtered off and the TFA solution was cooled and Et₂O solution was added. The precipitates were collected by centrifuging and decanting the ether layer (3 x). The resulting crude peptide is either redissolved directly into DMF or DMSO or CH₃CN/H₂O for purification by preparative HPLC or used directly in the next step.

### Analytical Data:

Mass Spectrometry: “ESI-MS(+)” signifies electrospray ionization mass spectrometry performed in positive ion mode; “ESI-MS (−)” signifies electrospray ionization mass spectrometry performed in negative ion mode; “ESI-HRMS(+)” signifies high-resolution electrospray ionization mass spectrometry performed in positive ion mode; “ESI-HRMS (−)” signifies high-resolution electrospray ionization mass spectrometry performed in negative ion mode. The detected masses are reported following the “m z” unit designation. Compounds with exact masses greater than 1000 were often detected as double-charged or triple-charged ions.

The crude material was purified via preparative LC/MS. Fractions containing the desired product were combined and dried via centrifugal evaporation.

The following abbreviations are employed in the Examples and elsewhere herein:

| | |
|---|---|
| μL | microliter |
| aq. | aqueous |
| Boc or BOC | tert-butyloxycarbonyl |
| Bn | benzyl |
| Bu | n-butyl |
| Bip | biphenyl |
| DCM | dichloromethane |
| DMAP | 4-(dimethylamino)pyridine |
| DIPEA | diisopropylethylamine |
| DMF | N,N-dimethylformamide |
| Et | ethyl |
| EDC | 1-ethyl-3-(3- |
| | dimethylaminopropyl)carbodiimide |
| Et₂O | diethyl ether |
| EtOAc | ethyl acetate |
| Fmoc or FMOC | fluorenylmethyloxycarbonyl |
| g | gram(s) |
| HOAc or AcOH | acetic acid |
| HATU | O-(7-Azabenzotriazol-1-yl)-1,1,3,3- |
| | tetramethyluronim hexafluorophosphate |
| HPLC | high performance liquid chromatography |
| h or hr | hour(s) |
| i-Bu | iso-butyl |
| i-Pr | iso-propyl |
| L | liter |
| LC/MS | high performance liquid |
| | chromatography/mass spectrometry |
| Me | methyl |
| min. | minute(s) |
| mL or ml | milliliter |
| MS or Mass Spec | mass spectrometry |
| mg | milligram(s) |
| mol | mole(s) |
| mmol | millimole(s) |
| mp | melting point |
| NMM | N-methylmorpholine |
| NMP | N-methylpyrrolidone |
| NMR | nuclear magnetic resonance |
| Pr | n-propyl |
| Ph | phenyl |
| pbf | 2,2,4,6,7-pentamethyIdlhydrobenzofuran-5- |
| | sulfonyl |
| rt or RT | room temperature |
| sat or sat'd | saturated |
| t-Bu | tert-butyl |
| Trt | trityl |
| TEA | triethylamine |
| TMS | trimethylsilyl |
| TIS | triisopropylsilane |
| THF | tetrahydrofuran |
| TFA | TFA |

### METHODS OF PREPARATION

All manipulations were performed under automation on a Prelude, Symphony, or Symphony X peptide synthesizer (Protein Technologies). Unless noted, all procedures were performed in a 45-mL polypropylene reaction vessel fitted with a bottom frit. The reaction vessel connects to the Prelude peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N2 gas through the bottom frit; the pulse lasts approximately 5 seconds and occurs every 30 seconds. Amino acid solutions were generally not used beyond two weeks from preparation. HATU solution was used within 7-14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl) (4-alkoxyphenyl) methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading. Fmoc-glycine-2-chlorotrityl chloride resin, 200-400 mesh, 1% DVB, 0.63 mmol/g loading.

Common amino acids used are listed below with side-chain protecting groups indicated inside parenthesis.

Fmoc-Ala-OH; Fmoc-Arg(Pbf)-OH; Fmoc-Asn(Trt)-OH; Fmoc-Asp(tBu)-OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH, Fmoc-Bip-OH and their corresponding D-amino acids.

Examples of Orthogonally Protected Amino Acids used in Solid Phase Synthesis

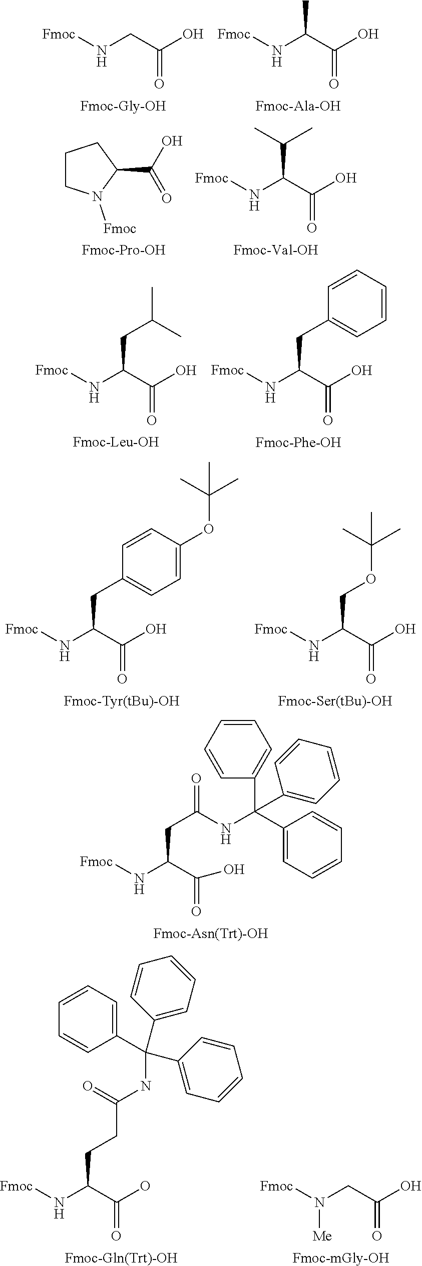

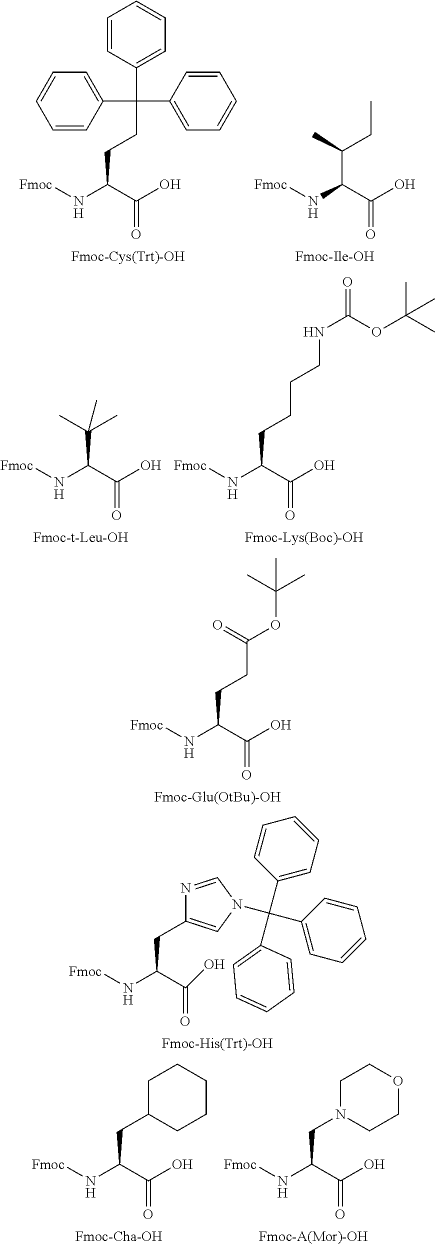

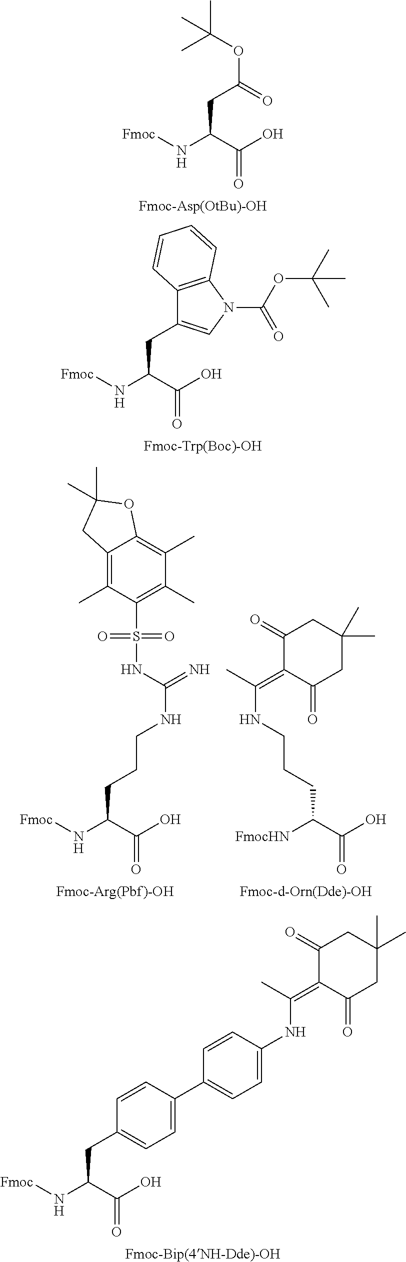

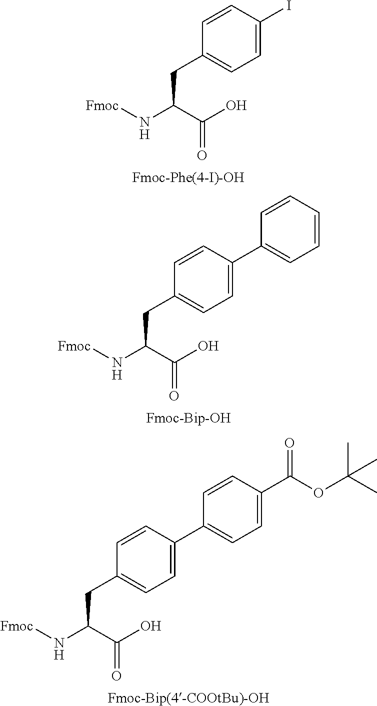

### Resin-Swelling Procedure:

To a 45-mL polypropylene solid-phase reaction vessel was added Sieber amide resin (140 mg, 0.100 mmol). The resin was washed (swelled) two times as follows: to the reaction vessel was added DMF (5.0 mL) through the top of the vessel “DMF top wash” upon which the mixture was periodically agitated for 10 minutes before the solvent was drained through the frit.

### Single-Coupling Procedure:

To the reaction vessel containing the resin from the previous step was added piperidine:DMF (20:80 v/v, 5.0 mL). The mixture was periodically agitated for 5.0 minutes and then the solution was drained through the frit. To the reaction vessel was added piperidine:DMF (20:80 v/v, 5.0 mL). The mixture was periodically agitated for 5.0 minutes and then the solution was drained through the frit. The resin was washed successively six times as follows: for each wash, DMF (6.0 mL) was added through the top of the vessel and the resulting mixture was periodically agitated for 1.0 minutes before the solution was drained through the frit. To the reaction vessel was added the amino acid (0.2 M in DMF, 4 equiv), then HATU (0.4 M in DMF, 4 equiv), and finally NMM (0.8 M in DMF, 8 equiv). The mixture was periodically agitated for 60-120 minutes, then the reaction solution was drained through the frit. The resin was washed successively four times as follows: for each wash, DMF (5.0 mL) was added through the top of the vessel and the resulting mixture was periodically agitated for 1.0 minute before the solution was drained through the frit. The resulting resin was used directly in the next step.

### Chloroacetic Anhydride Coupling:

To the reaction vessel containing the resin from the previous step was added piperidine:DMF (20:80 v/v, 3.0 mL). The mixture was periodically agitated for 5 minutes and then the solution was drained through the frit. To the reaction vessel was added piperidine:DMF (20:80 v/v, 3.0 mL). The mixture was periodically agitated for 5 minutes and then the solution was drained through the frit. The resin was washed successively six times as follows: for each wash, DMF (3.0 mL) was added through the top of the vessel and the resulting mixture was periodically agitated for one minute before the solution was drained through the frit. To the reaction vessel was added the chloroacetic anhydride solution (0.2 M in DMF, 2.5 mL, 10 equiv), then NMM (0.4 M in DMF, 2.5 mL, 20 equiv). The mixture was periodically agitated for 15 minutes, then the reaction solution was drained through the frit. The resin was washed twice as follows: for each wash, DMF (3.0 mL) was added through the top of the vessel and the resulting mixture was periodically agitated for one minute before the solution was drained through the frit. To the reaction vessel was added the chloroacetic anhydride solution (0.2 M in DMF, 2.5 mL, 10 equiv), then NMM (0.4 M in DMF, 5.0 mL, 20 equiv). The mixture was periodically agitated for 15 minutes, then the reaction solution was drained through the frit. The resin was washed successively five times as follows: for each wash, DMF (3.0 mL) was added through the top of the vessel and the resulting mixture was periodically agitated for one minute before the solution was drained through the frit. The resin was washed successively six times as follows: for each wash, DCM (5.0 mL) was added through the top of the vessel and the resulting mixture was periodically agitated for 0.5 minutes before the solution was drained through the frit. The resin was then dried with nitrogen flow for 10 minutes. The resulting resin was used directly in the next step.

### Global Deprotection:

Unless noted, all manipulations were performed manually. The procedure of “Global Deprotection Method” describes an experiment performed on a 0.050 mmol scale, where the scale is determined by the amount of Rink or Wang or chlorotrityl resin. The procedure can be scaled beyond 0.05 mmol scale by adjusting the described volumes by the multiple of the scale. In a 50-mL falcon tube was added the resin and 5.0 mL of the cleavage cocktail (TFA:TIS:DTT, v/v/w=95:5:0.5). The volume of the cleavage cocktail used for each individual linear peptide can be variable. Generally, higher number of protecting groups present in the sidechain of the peptide requires larger volume of the cleavage cocktail. The mixture was shaken at rt for 1-2 hours, usually about 1 hour. To the suspension was added 45-50 mL of cold diethyl ether. The mixture was vigorously mixed upon which a significant amount of a white solid precipitated. The mixture was centrifuged for 4 minutes, then the solution was decanted away from the solids and discarded. The solids were suspended in Et₂O (40 mL); then the mixture was centrifuged for 4 minutes; and the solution was decanted away from the solids and discarded to afford the crude peptide as a white to off-white solid together with the cleaved resin after drying under a flow of nitrogen and/or under house vacuum. The crude was used at the same day for the cyclization step.

### Cyclization Method:

Unless noted, all manipulations were performed manually. The procedure of describes an experiment performed on a 0.05 mmol scale, where the scale is determined by the amount of Rink or chlorotrityl or Wang resin that was used to generate the peptide. This scale is not based on a direct determination of the quantity of peptide used in the procedure. The procedure can be scaled beyond 0.05 mmol scale by adjusting the described volumes by the multiple of the scale. The crude peptide solids from the global deprotection were dissolved in DMF (35-40 mL) in the 50-mL centrifuge tube at rt, and to the solution was added DIPEA (2.0 mL) and the pH value of the reaction mixture above was 8. The solution was then allowed to shake overnight at rt. The reaction solution was concentrated to dryness on genevac EZ-2 and the crude residue was then dissolved in DMF or DMF/DMSO (2 mL). After filtration, this solution was subjected to single compound reverse-phase HPLC purification to afford the desired cyclic peptide.

Analysis Condition 1: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm).

Analysis Condition 2: Column: Waters XBridge C18, 2.1 mm×50 mm, 1.7 μm particles; Mobile Phase A: 5:95 acetonitrile:water with 0.1% TFA; Mobile Phase B: 95:5 acetonitrile:water with 0.1% TFA; Temperature: 50° C.; Gradient: 0% B to 100% B over 3 min, then a 0.50 min hold at 100% B; Flow: 1 mL/min; Detection: MS and UV (220 nm). Analysis Condition 3: Column: Waters Acquity BEH C18 1.7 um 2.1×50 mm, 1.7 μm particles; Mobile Phase A: 5:95 acetonitrile:water with 0.1% TFA; Mobile Phase B: 95:5 acetonitrile:water with 0.1% TFA; Temperature: 50° C.; Gradient: 0% B to 100% B over 1.5 min, then a 0.50 min hold at 100% B; Flow: 1 mL/min; Detection: MS and UV (220 nm). Analysis Condition 4: Column: Waters Acquity BEH C18 2.1×50 mm, 1.7 μm particles; Mobile Phase A: 5:95 acetonitrile:water with 0.1% TFA; Mobile Phase B: 95:5 acetonitrile:water with 0.1% TFA; Temperature: 50° C.; Gradient: 0% B to 100% B over 3.0 min, then a 0.50 min hold at 100% B; Flow: 1 mL/min; Detection: MS and UV (220 nm).

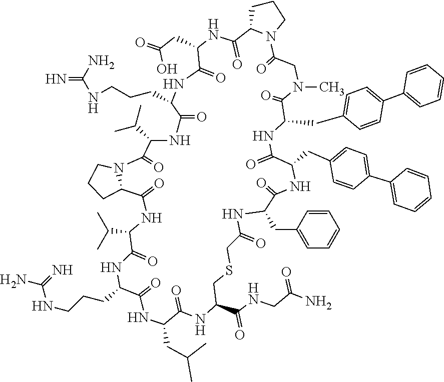 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

### Step 1: Synthesis of the Linear Sequence on Symphony X Peptide Synthesizer

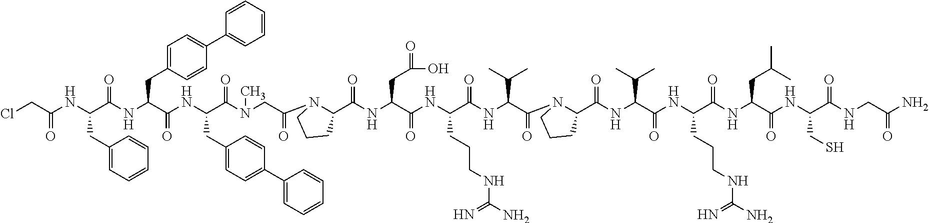 [SMILES: CC(C)C[C@H](NC(=O)[C@H](CCCNC(=N)N)NC(=O)[C@@H](NC(=O)[C@@H]1CCCN1C(=O)[C@@H](NC(=O)[C@H](CCCNC(=N)N)NC(=O)[C@H](CC(=O)O)NC(=O)[C@@H]1CCCN1C(=O)CN(C)C(=O)[C@H](Cc1ccc(-c2ccccc2)cc1)NC(=O)[C@H](Cc1ccc(-c2ccccc2)cc1)NC(=O)[C@H](Cc1ccccc1)NC(=O)CCl)C(C)C)C(C)C)C(=O)N[C@@H](CS)C(=O)NCC(N)=O]

To a 10-mL polypropylene solid-phase reaction vessel was added Fmoc-Rink Amide Resin (0.56 mmol/g, 89.3 mg), and the reaction vessel was placed on the Symphony X peptide synthesizer. The following procedures were then performed sequentially:
“Resin-swelling procedure” was followed;
“Single-coupling procedure” was followed with Fmoc-Gly-OH;
“Single-coupling procedure” was followed with Fmoc-Cys (Trt)-OH;
“Single-coupling procedure” was followed with Fmoc-Leu-OH;
“Single-coupling procedure” was followed with Fmoc-Arg (Pbf)-OH;
“Single-coupling procedure” was followed with Fmoc-Val-OH;
“Single-coupling procedure” was followed with Fmoc-Pro-OH;
“Single-coupling procedure” was followed with Fmoc-Val-OH;
“Single-coupling procedure” was followed with Fmoc-Arg (Pbf)-OH;
“Single-coupling procedure” was followed with Fmoc-Asp (OtBu)-OH;
“Single-coupling procedure” was followed with Fmoc-Pro-OH;
“Single-coupling procedure” was followed with Fmoc-mGly-OH;
“Single-coupling procedure” was followed with Fmoc-Bip-OH;
“Single-coupling procedure” was followed with Fmoc-Bip-OH;
“Single-coupling procedure” was followed with Fmoc-Phe-OH;
“Chloroacetic Anhydride Coupling”

Finally, the resin bound peptide was cleaved off the resin and deprotected following Global Deprotection Method procedure to provide crude linear peptide.

### Step 2. Macrocyclization:

The crude linear peptide, (0.05 mmol) was dissolved in DMF (38 mL). DIEA (2 mL) was added dropwise to adjust the pH to ˜8.3. The resulting mixture was shaken overnight at rt. The reaction was concentrated in vacuo, redissolved in DMF (2.0 mL), filtered through a 0.45-micron filter.

The crude material was purified via preparative LC/MS with the following conditions: Column: Waters XBridge C18, 19×250 mm, 5-μm particles; Mobile Phase A: 5:95 acetonitrile:water with 0.1% TFA; Mobile Phase B: 95:5 acetonitrile:water with 0.1% TFA; Gradient: 16-56% B over 20 minutes, and 0-minute hold at 100% B; Flow: 20 mL/min. Fractions containing the desired product were combined and dried via centrifugal evaporation to provide Example 1 (29.6 mg). Its estimated purity by LCMS analysis was 97.7%.

Analysis condition 1: Retention time=1.68 min. ESI-MS(+) m/z [M+H]⁺: 1815.1.

Examples 1 to 301, 320, 321, 334-342, 356-363, 385, 386, 396, 397, 450 to 460, 517 to 520, 522, 524, 577, 578, 604, 605 were prepared following analogous procedures described for Example 1.

### Preparation of Example 2

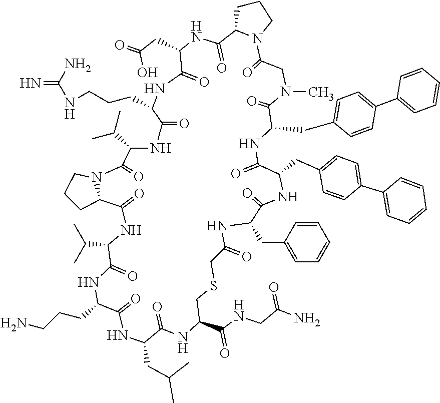 [SMILES: CC(C)C[C@@H]1NC(=O)[C@H](CCCN)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 2 was prepared on a 50 umol scale. It was purified by Preparative LC/MS. The yield of the product was 23.9 mg, and its estimated purity by LCMS analysis was 95.2%. Analysis condition 1: Retention time=1.61 min. ESI-MS(+) m/z [M+H]⁺: 1772.3.

### Preparation of Example 3

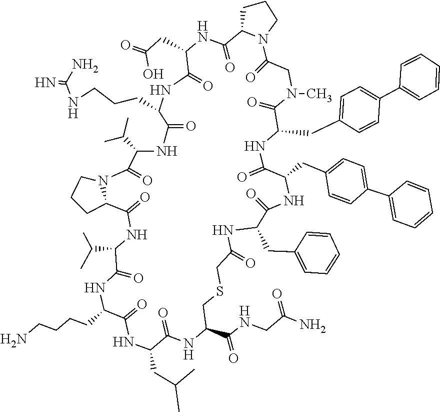 [SMILES: CC(C)C[C@@H]1NC(=O)[C@H](CCCCN)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 3 was prepared on a 50 umol scale. It was purified by Preparative LC/MS. The yield of the product was 10.1 mg, and its estimated purity by LCMS analysis was 97.9%. Analysis condition 1: Retention time=1.6 min. ESI-MS(+) m/z [M+H]⁺: 1788.2.

### Preparation of Example 4

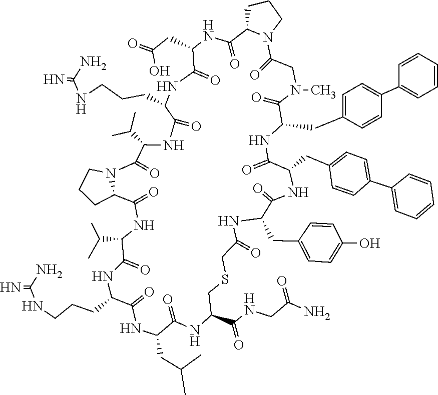 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccc(O)cc2)NC(=O)CSC[C@@H](C(=O)NCC(N)=O)NC1=O]

Example 4 was prepared on a 30 umol scale. It was purified by Preparative LC/MS. The yield of the product was 8.9 mg, and its estimated purity by LCMS analysis was 99.1%. Analysis condition 1: Retention time=1.54 min. ESI-MS(+) m/z [M+H]⁺: 1830.9.

### Preparation of Example 5

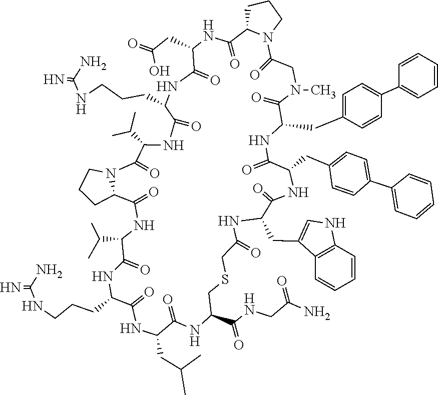 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2c[nH]c3ccccc23)NC(=O)CSC[C@@H](C(=O)NCC(N)=O)NC1=O]

Example 5 was prepared on a 30 umol scale. It was purified by Preparative LC/MS. The yield of the product was 12 mg, and its estimated purity by LCMS analysis was 100%. Analysis condition 2: Retention time=1.53 min. ESI-MS(+) m/z [M+H]⁺: 1854.

### Preparation of Example 6

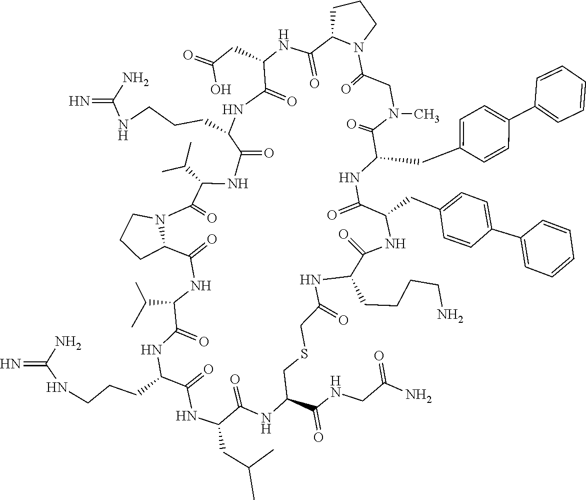 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CCCCN)NC(=O)CSC[C@@H](C(=O)NCC(N)=O)NC1=O]

Example 6 was prepared on a 30 umol scale. It was purified by Preparative LC/MS. The yield of the product was 2.6 mg, and its estimated purity by LCMS analysis was 95.6%. Analysis condition 1: Retention time=1.74 min. ESI-MS(+) m/z [M+2H]²⁺: 899.2.

### Preparation of Example 7

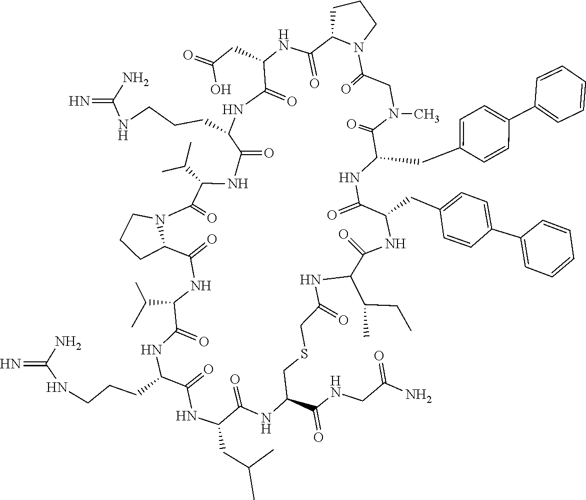 [SMILES: CC[C@H](C)C1NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC1=O]

Example 7 was prepared on a 30 umol scale. It was purified by Preparative LC/MS. The yield of the product was 6.7 mg, and its estimated purity by LCMS analysis was 100%. Analysis condition 2: Retention time=1.59 min. ESI-MS(+) m/z [M+2H]²⁺: 891.2.

### Preparation of Example 8

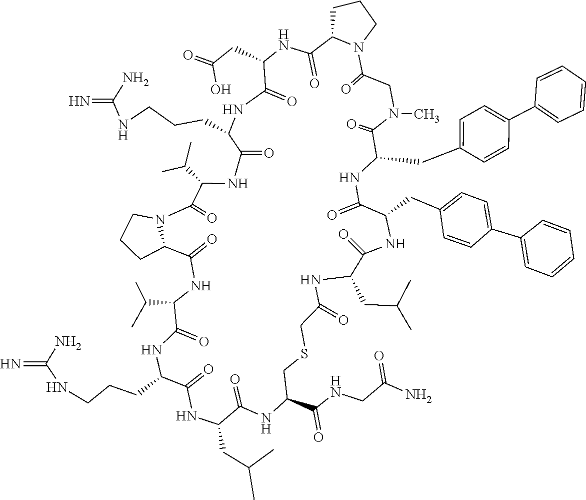 [SMILES: CC(C)C[C@@H]1NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC1=O]

Example 8 was prepared on a 30 umol scale. It was purified by Preparative LC/MS. The yield of the product was 1.9 mg, and its estimated purity by LCMS analysis was 97.5%. Analysis condition 2: Retention time=1.64 min. ESI-MS(+) m/z [M+H]⁺: 1781.6.

### Preparation of Example 9

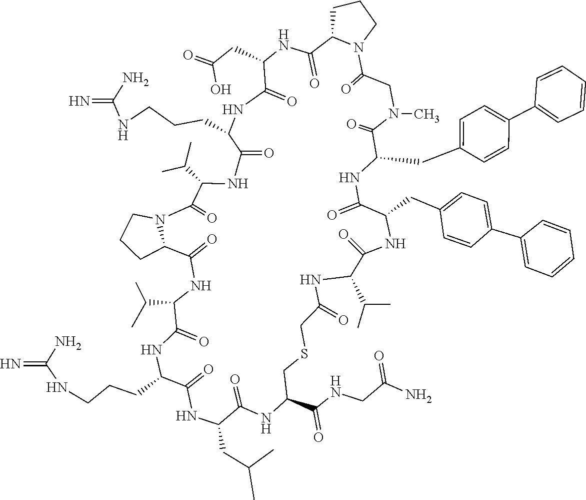 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](C(C)C)NC(=O)CSC[C@@H](C(=O)NCC(N)=O)NC1=O]

Example 9 was prepared on a 30 umol scale. It was purified by Preparative LC/MS. The yield of the product was 13.5 mg, and its estimated purity by LCMS analysis was 100%. Analysis condition 1: Retention time=1.57 min. ESI-MS(+) m/z [M+H]⁺: 1766.9.

### Preparation of Example 10

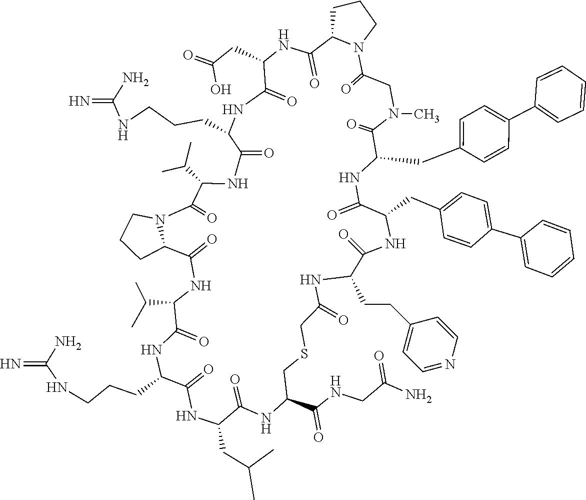 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CCc2ccncc2)NC(=O)CSC[C@@H](C(=O)NCC(N)=O)NC1=O]

Example 10 was prepared on a 30 umol scale. It was purified by Preparative LC/MS. The yield of the product was 9 mg, and its estimated purity by LCMS analysis was 100%. Analysis condition 4: Retention time=1.50 min. ESI-MS(+) m/z [M+H]⁺: 1831.07.

### Preparation of Example 11

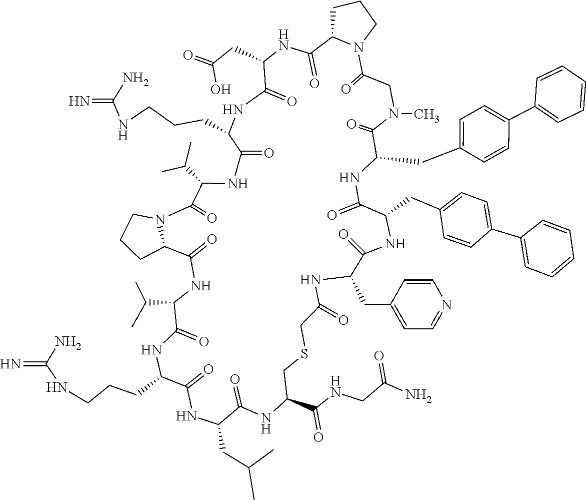 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccncc2)NC(=O)CSC[C@@H](C(=O)NCC(N)=O)NC1=O]

Example 11 was prepared on a 30 umol scale. It was purified by Preparative LC/MS. The yield of the product was 9.4 mg, and its estimated purity by LCMS analysis was 96.7%. Analysis condition 2: Retention time=1.51 min. ESI-MS(+) m/z [M+2H]²⁺: 908.2.

### Preparation of Example 12

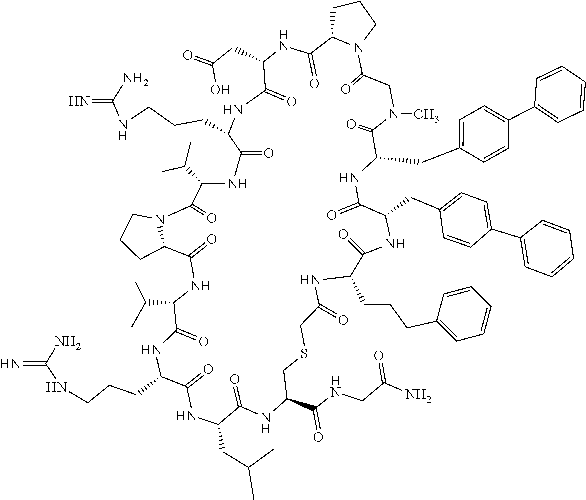 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CCCc2ccccc2)NC(=O)CSC[C@@H](C(=O)NCC(N)=O)NC1=O]

Example 12 was prepared on a 30 umol scale. It was purified by Preparative LC/MS. The yield of the product was 13.2 mg, and its estimated purity by LCMS analysis was 98%. Analysis condition 2: Retention time=1.73 min. ESI-MS(+) m/z [M+H]⁺: 1844.1.

### Preparation of Example 13

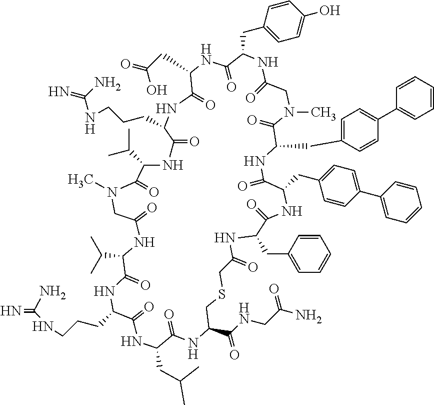 [SMILES: CC(C)C[C@@H]1NC(=O)[C@H](CCCNC(=N)N)NC(=O)[C@H](C(C)C)NC(=O)CN(C)C(=O)[C@H](C(C)C)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 13 was prepared on a 50 umol scale. It was purified by Preparative LC/MS. The yield of the product was 19.6 mg, and its estimated purity by LCMS analysis was 97.5%. Analysis condition 2: Retention time=1.72 min. ESI-MS(+) m/z [M+H]⁺: 1881.2.

### Preparation of Example 14

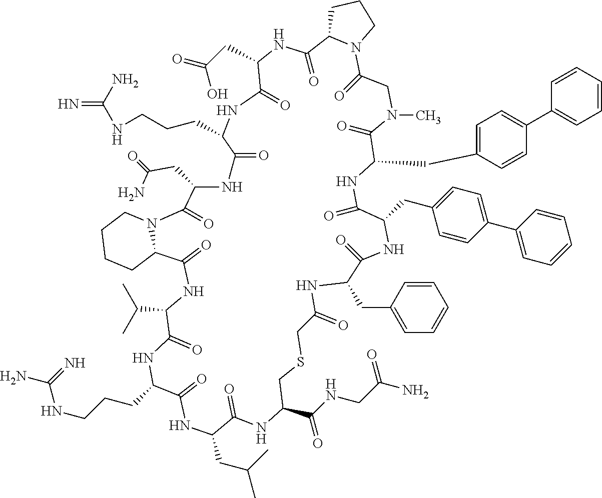 [SMILES: CC(C)C[C@@H]1NC(=O)[C@H](CCCNC(=N)N)NC(=O)[C@H](C(C)C)NC(=O)[C@@H]2CCCCN2C(=O)[C@H](CC(N)=O)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 14 was prepared on a 50 umol scale. It was purified by Preparative LC/MS. The yield of the product was 23.6 mg, and its estimated purity by LCMS analysis was 97.8%. Analysis condition 1: Retention time=1.59 min. ESI-MS(+) m/z [M+H]⁺: 1830.9.

### Preparation of Example 15

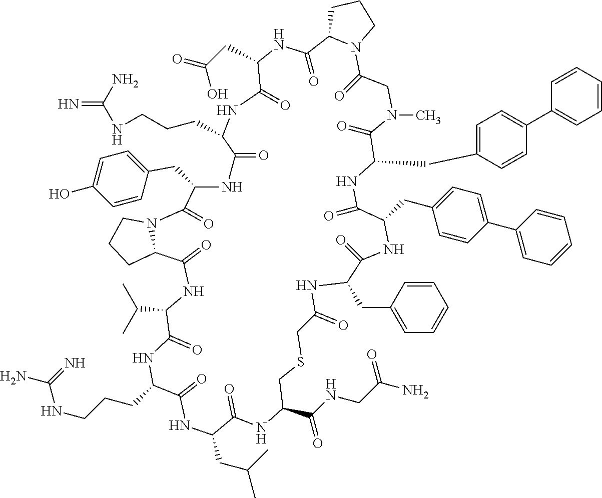 [SMILES: CC(C)C[C@@H]1NC(=O)[C@H](CCCNC(=N)N)NC(=O)[C@H](C(C)C)NC(=O)[C@@H]2CCCN2C(=O)[C@H](Cc2ccc(O)cc2)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 15 was prepared on a 50 umol scale. It was purified by Preparative LC/MS. The yield of the product was 24.5 mg, and its estimated purity by LCMS analysis was 95.6%. Analysis condition 1: Retention time=1.78 min. ESI-MS(+) m/z [M+H]⁺: 1880.2.

### Preparation of Example 16

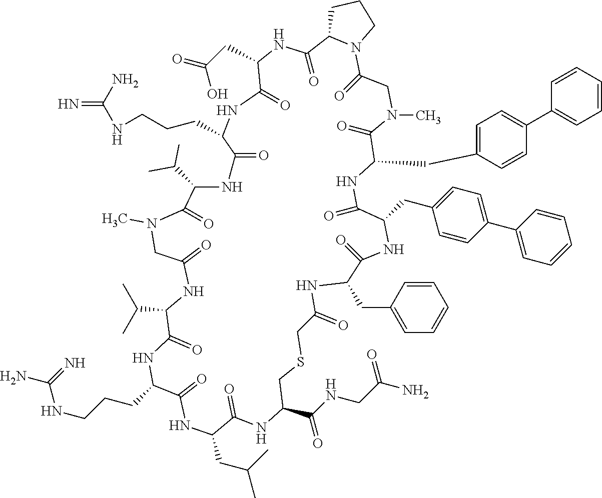 [SMILES: CC(C)C[C@@H]1NC(=O)[C@H](CCCNC(=N)N)NC(=O)[C@H](C(C)C)NC(=O)CN(C)C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 16 was prepared on a 50 umol scale. It was purified by Preparative LC/MS. The yield of the product was 22.5 mg, and its estimated purity by LCMS analysis was 96.1%. Analysis condition 2: Retention time=1.75 min. ESI-MS(+) m/z [M+H]⁺: 1790.

### Preparation of Example 17

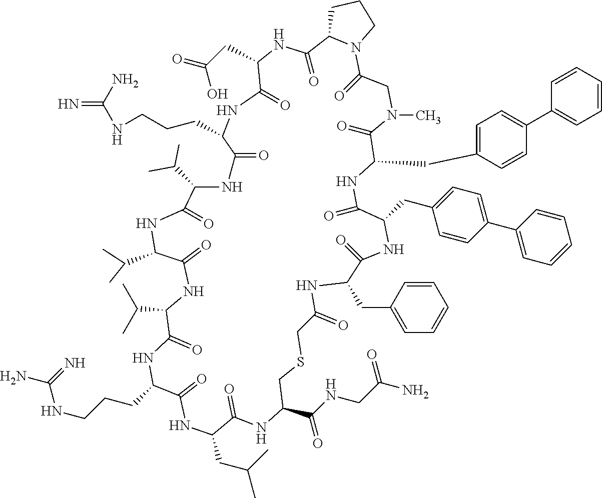 [SMILES: CC(C)C[C@@H]1NC(=O)[C@H](CCCNC(=N)N)NC(=O)[C@H](C(C)C)NC(=O)[C@H](C(C)C)NC(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 17 was prepared on a 50 umol scale. It was purified by Preparative LC/MS. The yield of the product was 7.9 mg, and its estimated purity by LCMS analysis was 98.2%. Analysis condition 2: Retention time=1.7 min. ESI-MS(+) m/z [M+H]⁺: 1818.1.

### Preparation of Example 18

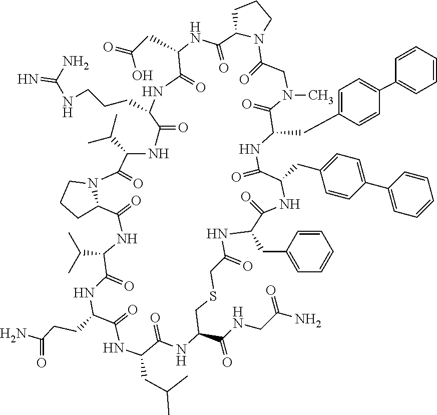 [SMILES: CC(C)C[C@@H]1NC(=O)[C@H](CCC(N)=O)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 18 was prepared on a 50 umol scale. It was purified by Preparative LC/MS. The yield of the product was 25.1 mg, and its estimated purity by LCMS analysis was 96.7%. Analysis condition 2: Retention time=1.74 min. ESI-MS(+) m/z [M+H]⁺: 1788.1.

### Preparation of Example 19

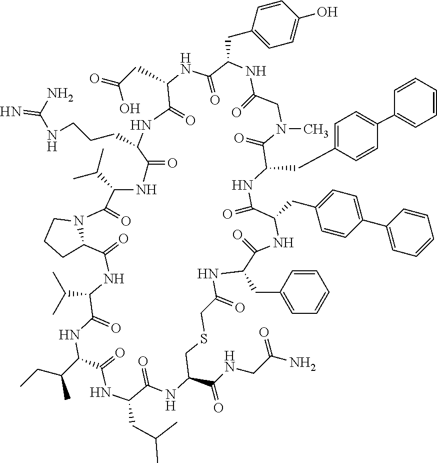 [SMILES: CC[C@H](C)[C@@H]1NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC(=O)[C@H](CC(C)C)NC1=O]

Example 19 was prepared on a 50 umol scale. It was purified by Preparative LC/MS. The yield of the product was 31.3 mg, and its estimated purity by LCMS analysis was 92.7%. Analysis condition 1: Retention time=1.89 min. ESI-MS(+) m/z [M+H]⁺: 1839.2.

### Preparation of Example 20

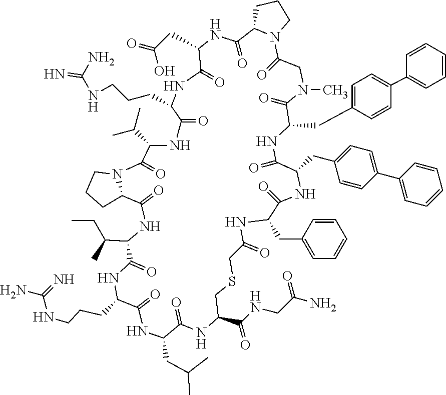 [SMILES: CC[C@H](C)[C@@H]1NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC(=O)[C@H](CC(C)C)NC(=O)[C@H](CCCNC(=N)N)NC1=O]

Example 20 was prepared on a 50 umol scale. It was purified by Preparative LC/MS. The yield of the product was 11.2 mg, and its estimated purity by LCMS analysis was 98.5%. Analysis condition 1: Retention time=1.7 min. ESI-MS(+) m/z [M+H]⁺: 1828.9.

### Preparation of Example 21

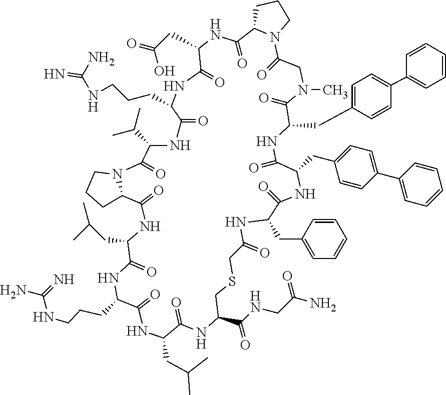 [SMILES: CC(C)C[C@@H]1NC(=O)[C@H](CCCNC(=N)N)NC(=O)[C@H](CC(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 21 was prepared on a 50 umol scale. It was purified by Preparative LC/MS. The yield of the product was 14.2 mg, and its estimated purity by LCMS analysis was 96.1%. Analysis condition 1: Retention time=1.81 min. ESI-MS(+) m/z [M+H]⁺: 1830.2.

### Preparation of Example 22

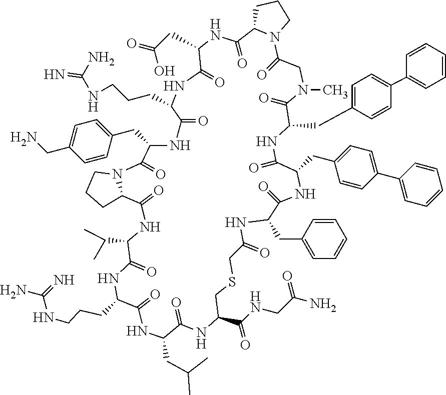 [SMILES: CC(C)C[C@@H]1NC(=O)[C@H](CCCNC(=N)N)NC(=O)[C@H](C(C)C)NC(=O)[C@@H]2CCCN2C(=O)[C@H](Cc2ccc(CN)cc2)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 22 was prepared on a 50 umol scale. It was purified by Preparative LC/MS. The yield of the product was 21 mg, and its estimated purity by LCMS analysis was 94.2%. Analysis condition 2: Retention time=1.52 min. ESI-MS(+) m/z [M+2H]²⁺: 947.2.

### Preparation of Example 23

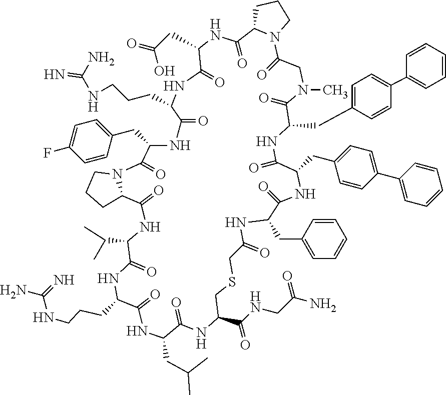 [SMILES: CC(C)C[C@@H]1NC(=O)[C@H](CCCNC(=N)N)NC(=O)[C@H](C(C)C)NC(=O)[C@@H]2CCCN2C(=O)[C@H](Cc2ccc(F)cc2)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 23 was prepared on a 50 umol scale. It was purified by Preparative LC/MS. The yield of the product was 36.5 mg, and its estimated purity by LCMS analysis was 98.3%. Analysis condition 1: Retention time=1.82 min. ESI-MS(+) m/z [M+2H]²⁺: 941.2.

### Preparation of Example 24

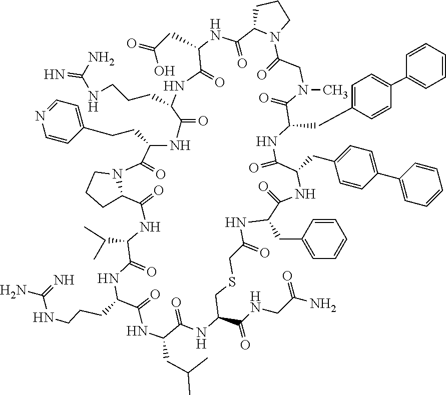 [SMILES: CC(C)C[C@@H]1NC(=O)[C@H](CCCNC(=N)N)NC(=O)[C@H](C(C)C)NC(=O)[C@@H]2CCCN2C(=O)[C@H](CCc2ccncc2)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 24 was prepared on a 50 umol scale. It was purified by Preparative LC/MS. The yield of the product was 17.6 mg, and its estimated purity by LCMS analysis was 96.4%. Analysis condition 1: Retention time=1.61 min. ESI-MS(+) m/z [M+H]⁺: 1877.9.

### Preparation of Example 25

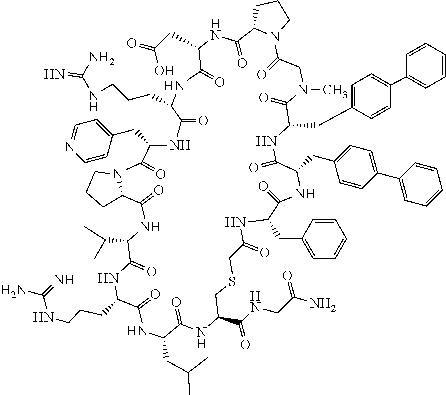 [SMILES: CC(C)C[C@@H]1NC(=O)[C@H](CCCNC(=N)N)NC(=O)[C@H](C(C)C)NC(=O)[C@@H]2CCCN2C(=O)[C@H](Cc2ccncc2)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 25 was prepared on a 50 umol scale. It was purified by Preparative LC/MS. The yield of the product was 295 mg, and its estimated purity by LCMS analysis was 94.2%. Analysis condition 2: Retention time=1.51 min. ESI-MS(+) m/z [M+H]⁺: 1864.8.

### Preparation of Example 26

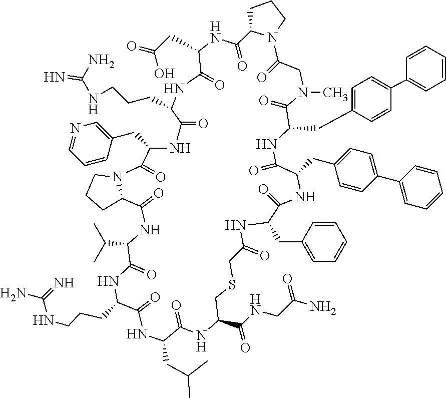 [SMILES: CC(C)C[C@@H]1NC(=O)[C@H](CCCNC(=N)N)NC(=O)[C@H](C(C)C)NC(=O)[C@@H]2CCCN2C(=O)[C@H](Cc2cccnc2)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 26 was prepared on a 50 umol scale. It was purified by Preparative LC/MS. The yield of the product was 22.8 mg, and its estimated purity by LCMS analysis was 93.5%. Analysis condition 2: Retention time=1.51 min. ESI-MS(+) m/z [M+2H]²⁺: 933.1.

### Preparation of Example 27

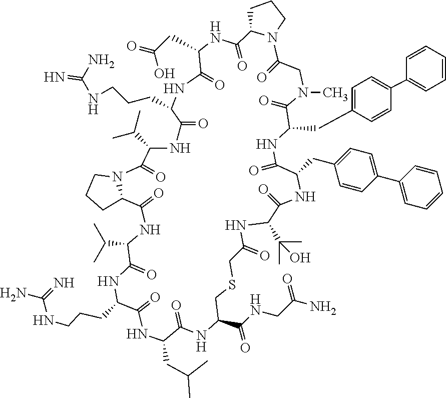 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](C(C)(C)O)NC(=O)CSC[C@@H](C(=O)NCC(N)=O)NC1=O]

Example 27 was prepared on a 50 umol scale. It was purified by Preparative LC/MS. The yield of the product was 18.3 mg, and its estimated purity by LCMS analysis was 96%. Analysis condition 2: Retention time=1.6 min. ESI-MS(+) m/z [M+H]⁺: 1784.1.

### Preparation of Example 27

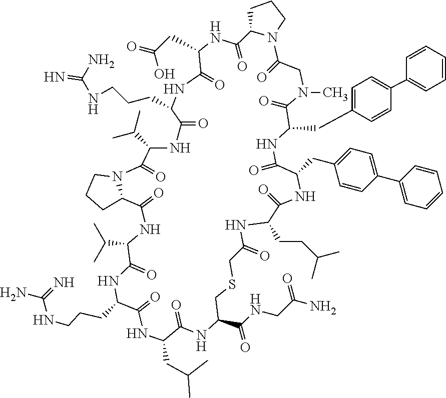 [SMILES: CC(C)CC[C@@H]1NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC1=O]

Example 28 was prepared on a 50 umol scale. It was purified by Preparative LC/MS. The yield of the product was 30.3 mg, and its estimated purity by LCMS analysis was 96.7%. Analysis condition 2: Retention time=1.71 min. ESI-MS(+) m/z [M+H]⁺: 1795.9.

### Preparation of Example 29

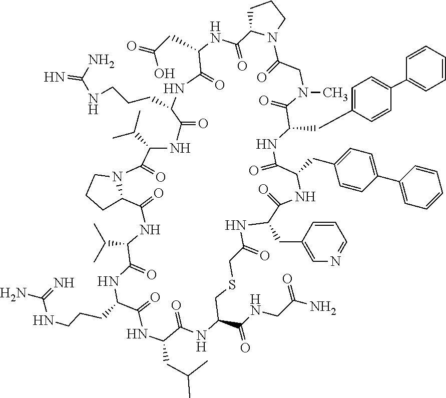 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2cccnc2)NC(=O)CSC[C@@H](C(=O)NCC(N)=O)NC1=O]

Example 29 was prepared on a 50 umol scale. It was purified by Preparative LC/MS. The yield of the product was 14.5 mg, and its estimated purity by LCMS analysis was 92.1%. Analysis condition 1: Retention time=1.65 min. ESI-MS(+) m/z [M+H]⁺: 1816.

### Preparation of Example 30

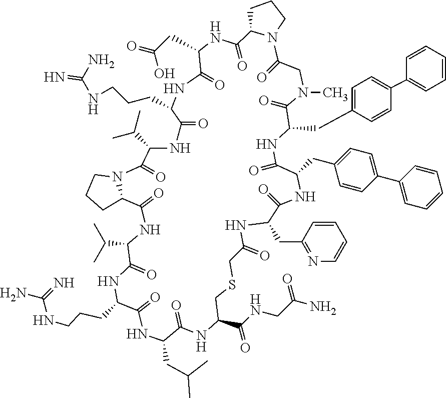 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccn2)NC(=O)CSC[C@@H](C(=O)NCC(N)=O)NC1=O]

Example 30 was prepared on a 50 umol scale. It was purified by Preparative LC/MS. The yield of the product was 20.9 mg, and its estimated purity by LCMS analysis was 93.4%. Analysis condition 1: Retention time=1.63 min. ESI-MS(+) m/z [M+2H]²⁺: 908.87.

### Preparation of Example 31

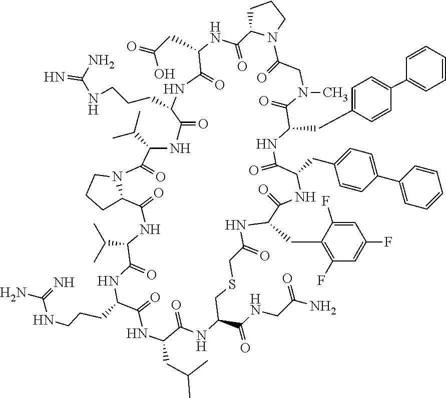 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2c(F)cc(F)cc2F)NC(=O)CSC[C@@H](C(=O)NCC(N)=O)NC1=O]

Example 31 was prepared on a 50 umol scale. It was purified by Preparative LC/MS. The yield of the product was 15.4 mg, and its estimated purity by LCMS analysis was 96.5%. Analysis condition 1: Retention time=1.7 min. ESI-MS(+) m/z [M+2H]²⁺: 935.4.

### Preparation of Example 32

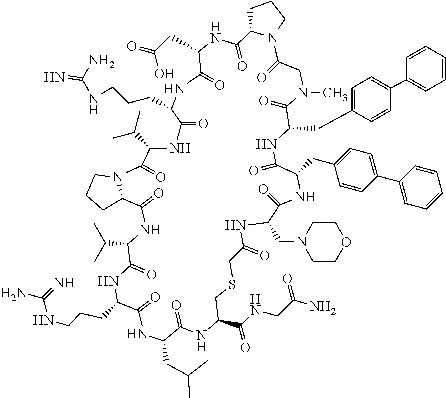 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 32 was prepared on a 50 umol scale. It was purified by Preparative LC/MS. The yield of the product was 13.2 mg, and its estimated purity by LCMS analysis was 96.7%. Analysis condition 1: Retention time=1.6 min. ESI-MS(+) m/z [M+H]⁺: 1824.2.

### Preparation of Example 33

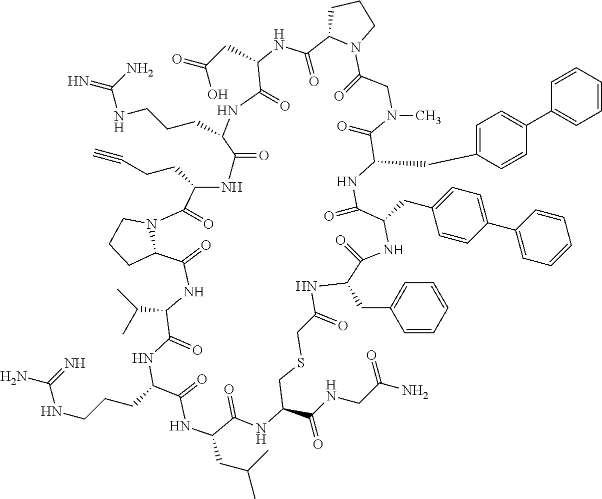 [SMILES: C#CCC[C@@H]1NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]2CCCN2C1=O]

Example 33 was prepared on a 50 umol scale. It was purified by Preparative LC/MS. The yield of the product was 19.9 mg, and its estimated purity by LCMS analysis was 100%. Analysis condition 1: Retention time=1.76 min. ESI-MS(+) m/z [M+2H]²⁺: 913.1.

### Preparation of Example 34

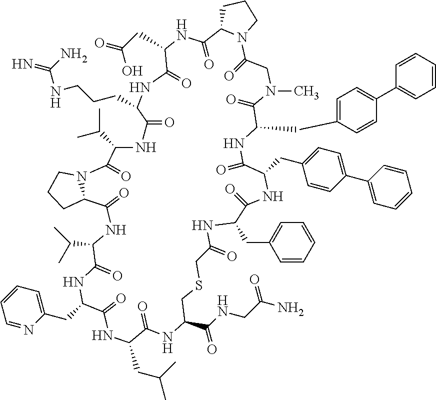 [SMILES: CC(C)C[C@@H]1NC(=O)[C@H](Cc2ccccn2)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 34 was prepared on a 50 umol scale. It was purified by Preparative LC/MS. The yield of the product was 15.2 mg, and its estimated purity by LCMS analysis was 97.5%. Analysis condition 2: Retention time=1.65 min. ESI-MS(+) m/z [M+H]⁺: 1808.1.

### Preparation of Example 35

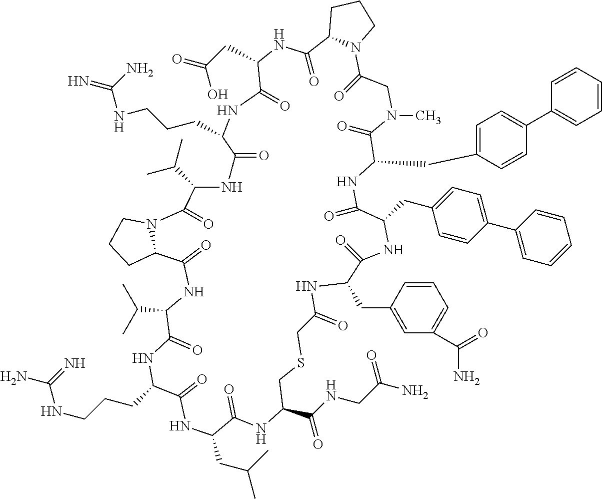 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2cccc(C(N)=O)c2)NC(=O)CSC[C@@H](C(=O)NCC(N)=O)NC1=O]

Example 35 was prepared on a 50 umol scale. It was purified by Preparative LC/MS. The yield of the product was 8.5 mg, and its estimated purity by LCMS analysis was 96.4%. Analysis condition 1: Retention time=1.49 min. ESI-MS(+) m/z [M+H]⁺: 1857.9.

### Preparation of Example 36

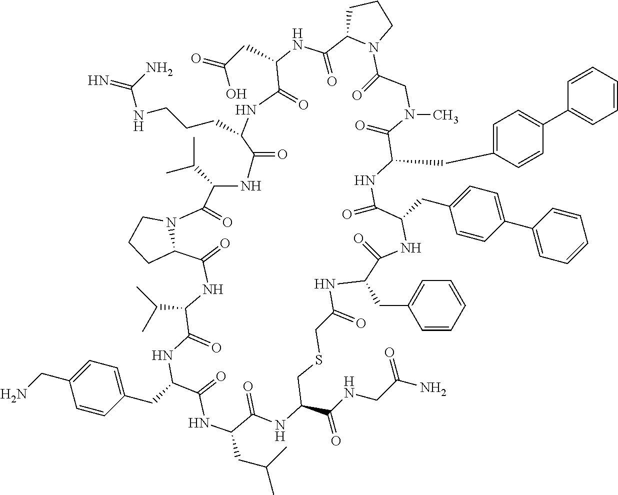 [SMILES: CC(C)C[C@@H]1NC(=O)[C@H](Cc2ccc(CN)cc2)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 36 was prepared on a 50 umol scale. It was purified by Preparative LC/MS. The yield of the product was 21.4 mg, and its estimated purity by LCMS analysis was 94.2%. Analysis condition 1: Retention time=1.62 min. ESI-MS(+) m/z [M+2H]²⁺: 918.3.

### Preparation of Example 37

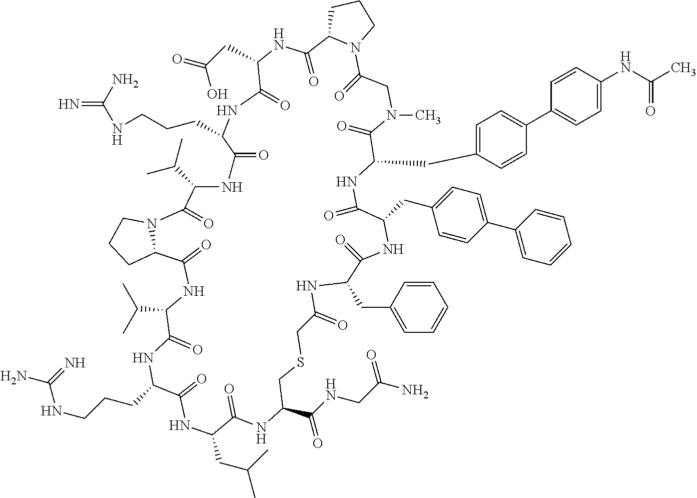 [SMILES: CC(=O)Nc1ccc(-c2ccc(C[C@@H]3NC(=O)[C@H](Cc4ccc(-c5ccccc5)cc4)NC(=O)[C@H](Cc4ccccc4)NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]4CCCN4C(=O)[C@H](C(C)C)NC(=O)[C@H](CCCNC(=N)N)NC(=O)[C@H](CC(=O)O)NC(=O)[C@@H]4CCCN4C(=O)CN(C)C3=O)cc2)cc1]

Example 37 was prepared on a 50 umol scale. It was purified by Preparative LC/MS. The yield of the product was 7 mg, and its estimated purity by LCMS analysis was 95.5%. Analysis condition 1: Retention time=1.47 min. ESI-MS(+) m/z [M+H]⁺: 1872.1.

### Preparation of Example 38

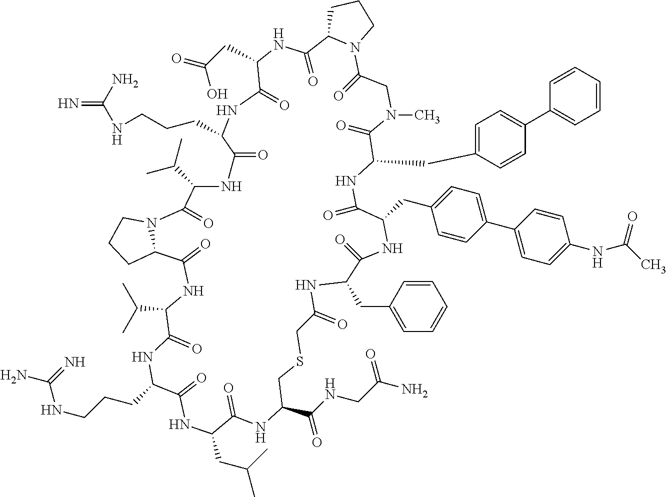 [SMILES: CC(=O)Nc1ccc(-c2ccc(C[C@@H]3NC(=O)[C@H](Cc4ccccc4)NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]4CCCN4C(=O)[C@H](C(C)C)NC(=O)[C@H](CCCNC(=N)N)NC(=O)[C@H](CC(=O)O)NC(=O)[C@@H]4CCCN4C(=O)CN(C)C(=O)[C@H](Cc4ccc(-c5ccccc5)cc4)NC3=O)cc2)cc1]

Example 38 was prepared on a 50 umol scale. It was purified by Preparative LC/MS. The yield of the product was 17.7 mg, and its estimated purity by LCMS analysis was 91%. Analysis condition 1: Retention time=1.51 min. ESI-MS(+) m/z [M+H]⁺: 1872.1.

### Preparation of Example 39

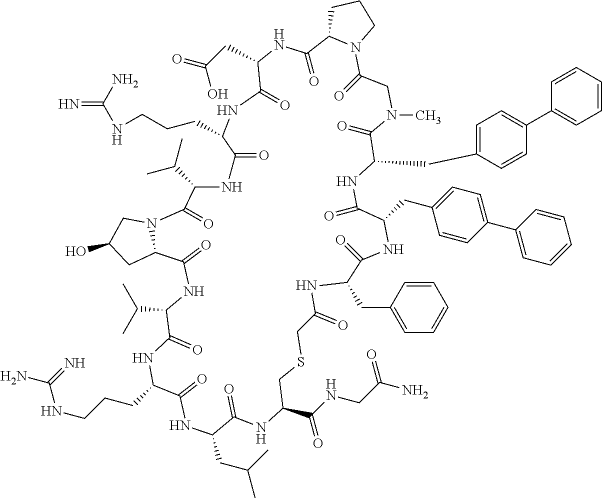 [SMILES: CC(C)C[C@@H]1NC(=O)[C@H](CCCNC(=N)N)NC(=O)[C@H](C(C)C)NC(=O)[C@@H]2C[C@@H](O)CN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 39 was prepared on a 50 umol scale. It was purified by Preparative LC/MS. The yield of the product was 16.7 mg, and its estimated purity by LCMS analysis was 98.5%. Analysis condition 1: Retention time=1.72 min. ESI-MS(+) m/z [M+H]⁺: 1831.

### Preparation of Example 40

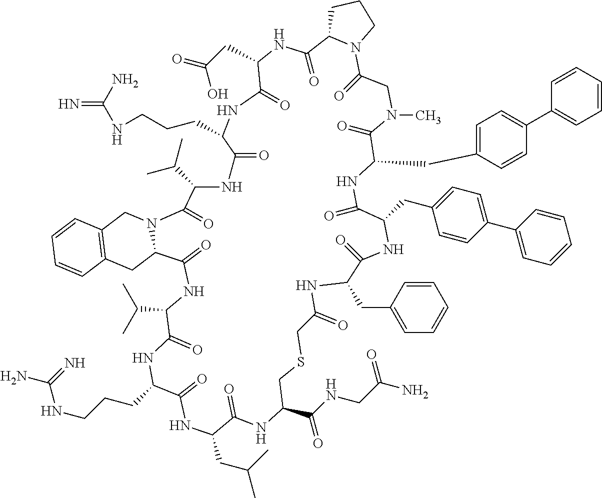 [SMILES: CC(C)C[C@@H]1NC(=O)[C@H](CCCNC(=N)N)NC(=O)[C@H](C(C)C)NC(=O)[C@@H]2Cc3ccccc3CN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 40 was prepared on a 50 umol scale. It was purified by Preparative LC/MS. The yield of the product was 18.7 mg, and its estimated purity by LCMS analysis was 98.4%. Analysis condition 2: Retention time=1.76 min. ESI-MS(+) m/z [M+H]⁺: 1878.2.

### Preparation of Example 41

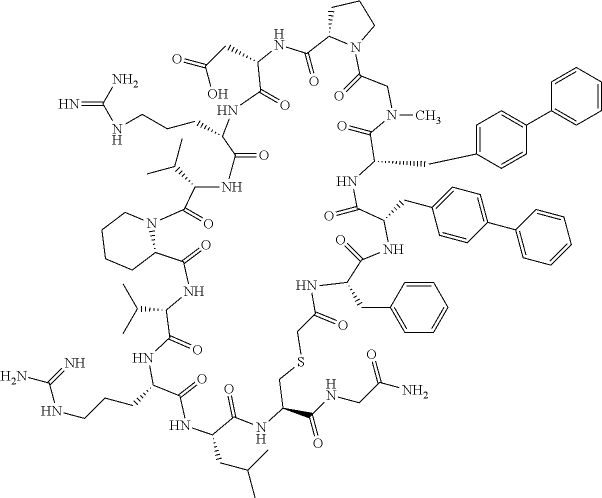 [SMILES: CC(C)C[C@@H]1NC(=O)[C@H](CCCNC(=N)N)NC(=O)[C@H](C(C)C)NC(=O)[C@@H]2CCCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 41 was prepared on a 50 umol scale. It was purified by Preparative LC/MS. The yield of the product was 12 mg, and its estimated purity by LCMS analysis was 99.3%. Analysis condition 2: Retention time=1.69 min. ESI-MS(+) m/z [M+2H] 2+: 915.3.

### Preparation of Example 42

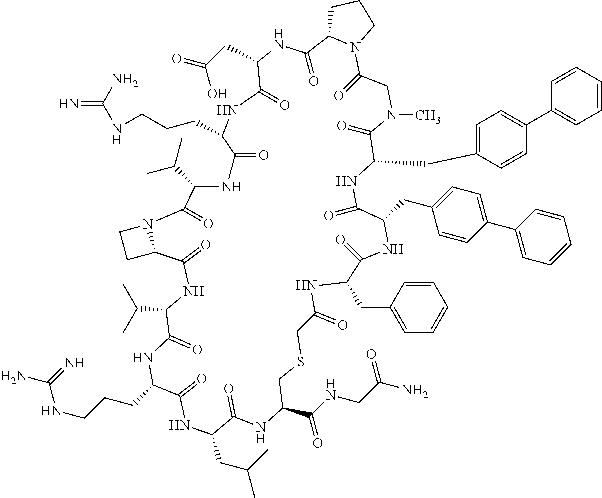 [SMILES: CC(C)C[C@@H]1NC(=O)[C@H](CCCNC(=N)N)NC(=O)[C@H](C(C)C)NC(=O)[C@@H]2CCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 42 was prepared on a 50 umol scale. It was purified by Preparative LC/MS. The yield of the product was 31.2 mg, and its estimated purity by LCMS analysis was 93.4%. Analysis condition 2: Retention time=1.78 min. ESI-MS(+) m/z [M+2H] 2+: 901.4.

### Preparation of Example 43

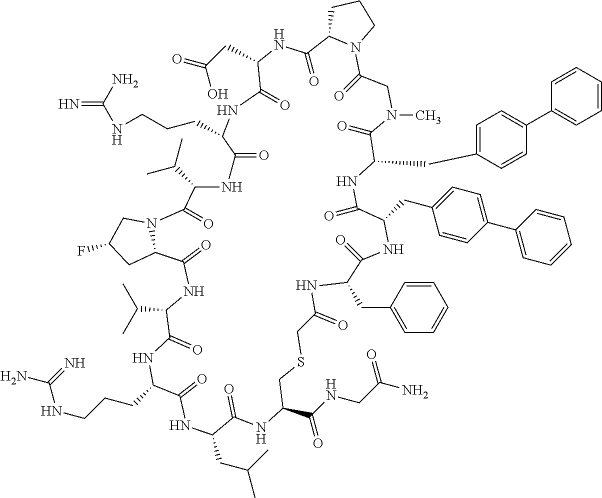 [SMILES: CC(C)C[C@@H]1NC(=O)[C@H](CCCNC(=N)N)NC(=O)[C@H](C(C)C)NC(=O)[C@@H]2C[C@H](F)CN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 43 was prepared on a 50 umol scale. It was purified by Preparative LC/MS. The yield of the product was 31.5 mg, and its estimated purity by LCMS analysis was 95.3%. Analysis condition 2: Retention time=1.75 min. ESI-MS(+) m/z [M+H]⁺: 1833.2.

### Preparation of Example 44

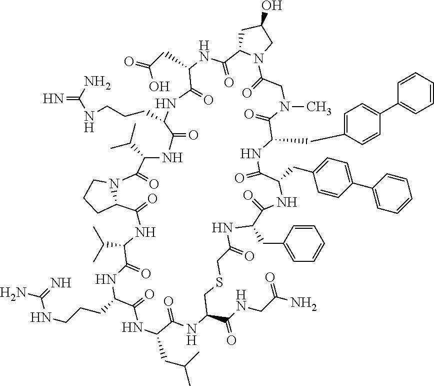 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2C[C@@H](O)CN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 44 was prepared on a 50 umol scale. It was purified by Preparative LC/MS 37. The yield of the product was 23.1 mg, and its estimated purity by LCMS analysis was 94.1%. Analysis condition 1: Retention time=1.61 min. ESI-MS(+) m/z [M+H]⁺: 1830.9.

### Preparation of Example 45

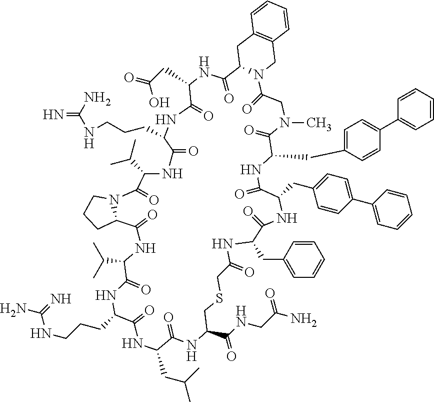 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2Cc3ccccc3CN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 45 was prepared on a 50 umol scale. It was purified by Preparative LC/MS. The yield of the product was 24.4 mg, and its estimated purity by LCMS analysis was 97.9%. Analysis condition 2: Retention time=1.86 min. ESI-MS(+) m/z [M+H]⁺: 1877.1.

### Preparation of Example 46

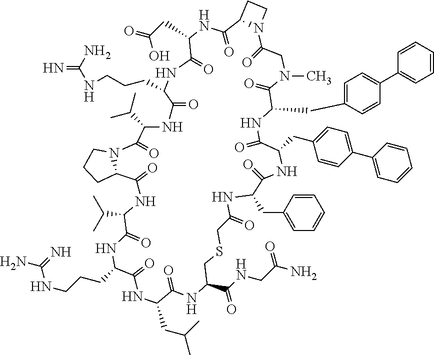 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCN2C(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 46 was prepared on a 50 umol scale. It was purified by Preparative LC/MS. The yield of the product was 16.8 mg, and its estimated purity by LCMS analysis was 98.7%. Analysis condition 2: Retention time=1.67 min. ESI-MS(+) m/z [M+H]⁺: 1802.07.

### Preparation of Example 47

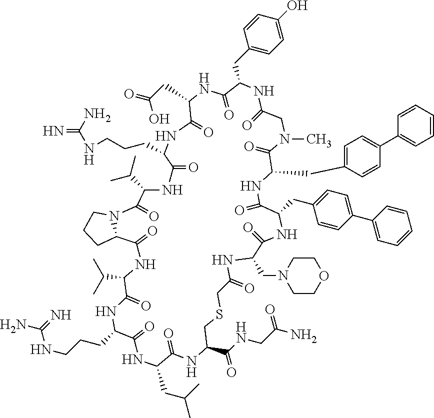 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 47 was prepared on a 50 umol scale. It was purified by Preparative LC/MS. The yield of the product was 30.2 mg, and its estimated purity by LCMS analysis was 96.4%. Analysis condition 2: Retention time=1.56 min. ESI-MS(+) m/z [M+2H] 2+: 945.9.

### Preparation of Example 48

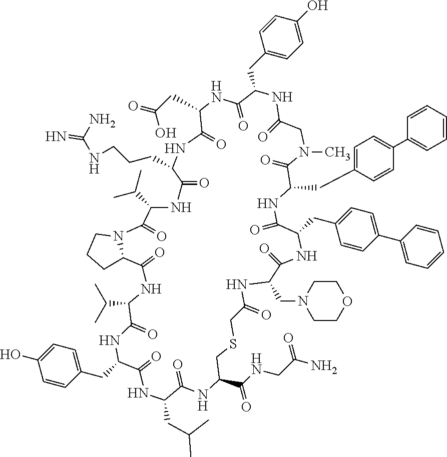 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 48 was prepared on a 50 umol scale. It was purified by Preparative LC/MS. The yield of the product was 34.8 mg, and its estimated purity by LCMS analysis was 93.5%. Analysis condition 1: Retention time=1.74 min. ESI-MS(+) m/z [M+H]⁺: 1897.

### Preparation of Example 49

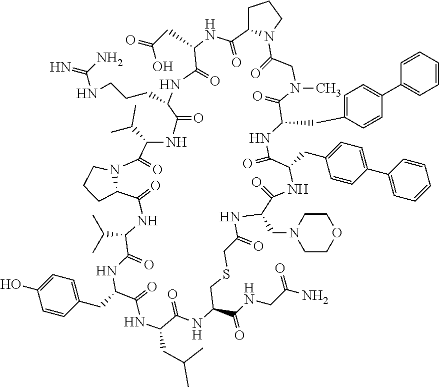 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 49 was prepared on a 50 umol scale. It was purified by Preparative LC/MS. The yield of the product was 40.2 mg, and its estimated purity by LCMS analysis was 93.5%. Analysis condition 1: Retention time=1.71 min. ESI-MS(+) m/z [M+H]⁺: 1831.

### Preparation of Example 50

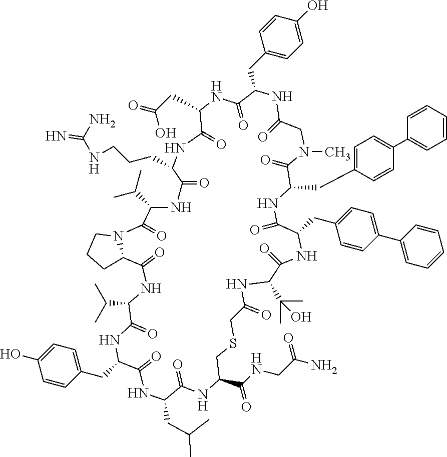 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](C(C)(C)O)NC(=O)CSC[C@@H](C(=O)NCC(N)=O)NC1=O]

Example 50 was prepared on a 50 umol scale. It was purified by Preparative LC/MS. The yield of the product was 34.1 mg, and its estimated purity by LCMS analysis was 92.8%. Analysis condition 1: Retention time=1.78 min. ESI-MS(+) m/z [M+2H] 2+: 928.2.

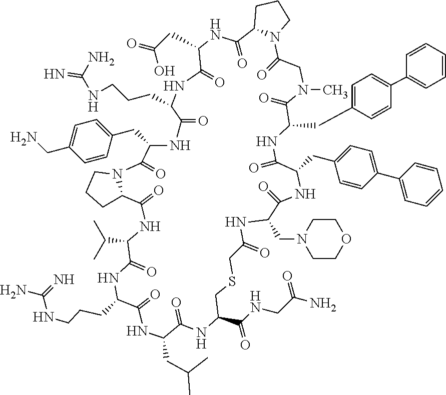 [SMILES: CC(C)C[C@@H]1NC(=O)[C@H](CCCNC(=N)N)NC(=O)[C@H](C(C)C)NC(=O)[C@@H]2CCCN2C(=O)[C@H](Cc2ccc(CN)cc2)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 51 was prepared on a 50 umol scale. It was purified by Preparative LC/MS. The yield of the product was 31.1 mg, and its estimated purity by LCMS analysis was 95.5%. Analysis condition 2: Retention time=1.43 min. ESI-MS(+) m/z [M+H]⁺: 1901.2.

### Preparation of Example 52

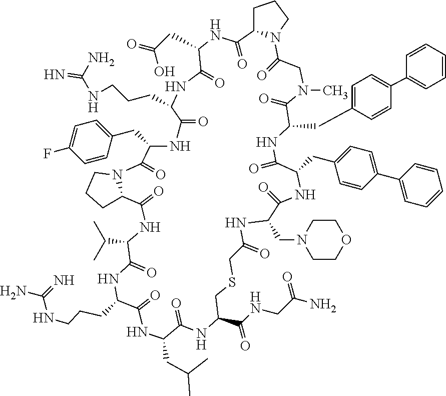 [SMILES: CC(C)C[C@@H]1NC(=O)[C@H](CCCNC(=N)N)NC(=O)[C@H](C(C)C)NC(=O)[C@@H]2CCCN2C(=O)[C@H](Cc2ccc(F)cc2)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 52 was prepared on a 50 umol scale. It was purified by Preparative LC/MS. The yield of the product was 33.9 mg, and its estimated purity by LCMS analysis was 95%. Analysis condition 1: Retention time=1.76 min. ESI-MS(+) m/z [M+H]⁺: 1890.1.

### Preparation of Example 53

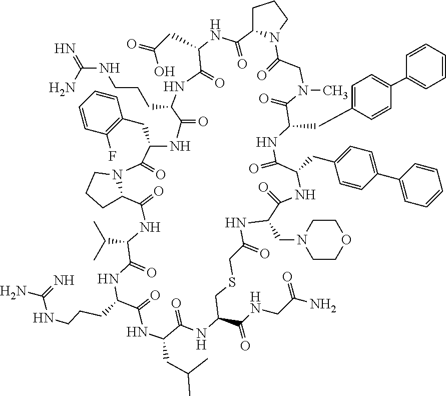 [SMILES: CC(C)C[C@@H]1NC(=O)[C@H](CCCNC(=N)N)NC(=O)[C@H](C(C)C)NC(=O)[C@@H]2CCCN2C(=O)[C@H](Cc2ccccc2F)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 53 was prepared on a 50 umol scale. It was purified by Preparative LC/MS. The yield of the product was 22.7 mg, and its estimated purity by LCMS analysis was 92.6%. Analysis condition 2: Retention time=1.64 min. ESI-MS(+) m/z [M+2H] 2+: 946.

### Preparation of Example 54

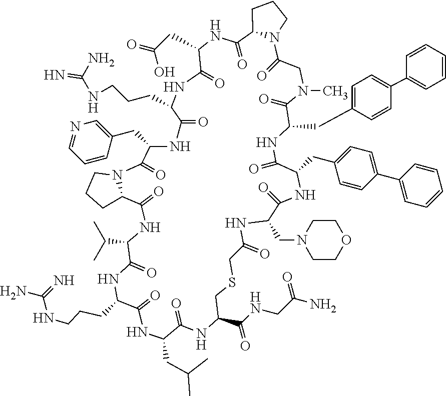 [SMILES: CC(C)C[C@@H]1NC(=O)[C@H](CCCNC(=N)N)NC(=O)[C@H](C(C)C)NC(=O)[C@@H]2CCCN2C(=O)[C@H](Cc2cccnc2)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 54 was prepared on a 50 umol scale. It was purified by Preparative LC/MS. The yield of the product was 22 mg, and its estimated purity by LCMS analysis was 95.8%. Analysis condition 1: Retention time=1.64 min. ESI-MS(+) m/z [M+H]⁺: 1872.9.

### Preparation of Example 55

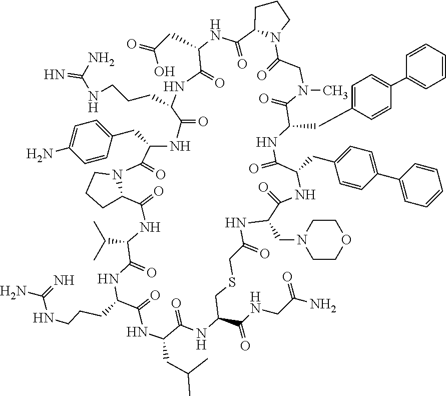 [SMILES: CC(C)C[C@@H]1NC(=O)[C@H](CCCNC(=N)N)NC(=O)[C@H](C(C)C)NC(=O)[C@@H]2CCCN2C(=O)[C@H](Cc2ccc(N)cc2)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 55 was prepared on a 50 umol scale. It was purified by Preparative LC/MS. The yield of the product was 24.6 mg, and its estimated purity by LCMS analysis was 98.6%. Analysis condition 1: Retention time=1.76 min. ESI-MS(+) m/z [M+H]⁺: 1888.2.

### Preparation of Example 56

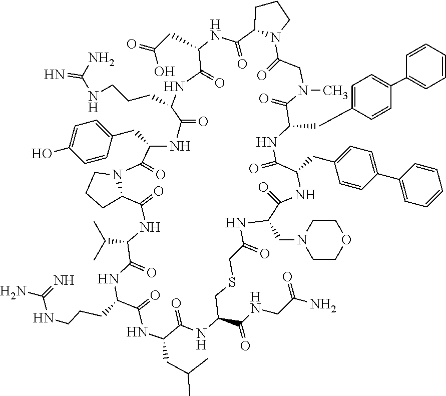 [SMILES: CC(C)C[C@@H]1NC(=O)[C@H](CCCNC(=N)N)NC(=O)[C@H](C(C)C)NC(=O)[C@@H]2CCCN2C(=O)[C@H](Cc2ccc(O)cc2)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 56 was prepared on a 50 umol scale. It was purified by Preparative LC/MS. The yield of the product was 21.9 mg, and its estimated purity by LCMS analysis was 94.6%. Analysis condition 1: Retention time=1.76 min. ESI-MS(+) m/z [M+H]⁺: 1888.1.

### Preparation of Example 57

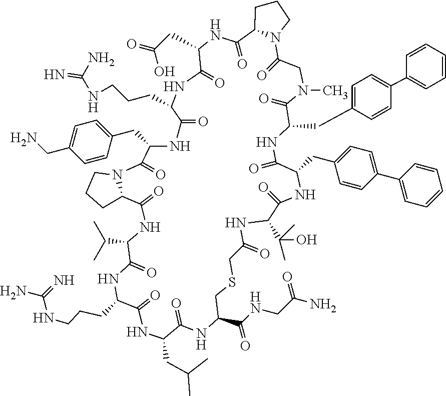 [SMILES: CC(C)C[C@@H]1NC(=O)[C@H](CCCNC(=N)N)NC(=O)[C@H](C(C)C)NC(=O)[C@@H]2CCCN2C(=O)[C@H](Cc2ccc(CN)cc2)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](C(C)(C)O)NC(=O)CSC[C@@H](C(=O)NCC(N)=O)NC1=O]

Example 57 was prepared on a 50 umol scale. It was purified by Preparative LC/MS. The yield of the product was 20 mg, and its estimated purity by LCMS analysis was 95.3%. Analysis condition 1: Retention time=1.5 min. ESI-MS(+) m/z [M+H]⁺: 1860.2.

### Preparation of Example 58

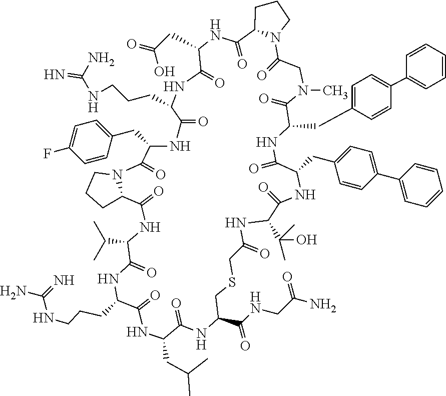 [SMILES: CC(C)C[C@@H]1NC(=O)[C@H](CCCNC(=N)N)NC(=O)[C@H](C(C)C)NC(=O)[C@@H]2CCCN2C(=O)[C@H](Cc2ccc(F)cc2)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](C(C)(C)O)NC(=O)CSC[C@@H](C(=O)NCC(N)=O)NC1=O]

Example 58 was prepared on a 50 umol scale. It was purified by Preparative LC/MS. The yield of the product was 19.9 mg, and its estimated purity by LCMS analysis was 98.1%. Analysis condition 2: Retention time=1.68 min. ESI-MS(+) m/z [M+2H] 2+: 925.5.

### Preparation of Example 59

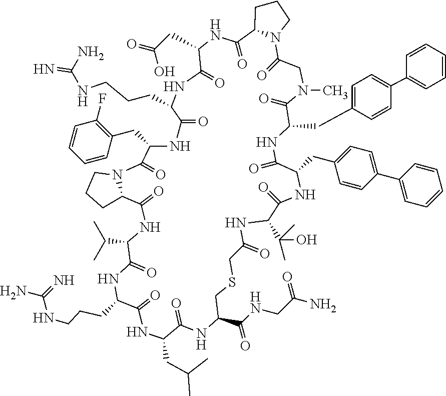 [SMILES: CC(C)C[C@@H]1NC(=O)[C@H](CCCNC(=N)N)NC(=O)[C@H](C(C)C)NC(=O)[C@@H]2CCCN2C(=O)[C@H](Cc2ccccc2F)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](C(C)(C)O)NC(=O)CSC[C@@H](C(=O)NCC(N)=O)NC1=O]

Example 59 was prepared on a 50 umol scale. It was purified by Preparative LC/MS. The yield of the product was 13.9 mg, and its estimated purity by LCMS analysis was 95.8%. Analysis condition 2: Retention time=1.68 min. ESI-MS(+) m/z [M+H]⁺: 1849.9.

### Preparation of Example 60

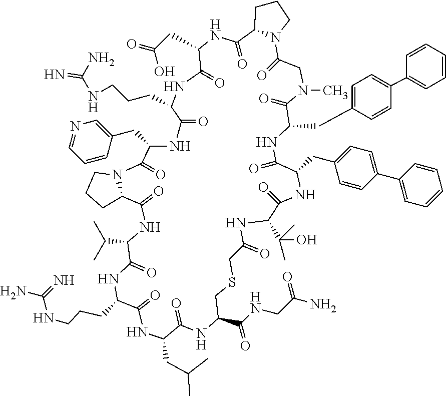 [SMILES: CC(C)C[C@@H]1NC(=O)[C@H](CCCNC(=N)N)NC(=O)[C@H](C(C)C)NC(=O)[C@@H]2CCCN2C(=O)[C@H](Cc2cccnc2)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](C(C)(C)O)NC(=O)CSC[C@@H](C(=O)NCC(N)=O)NC1=O]

Example 60 was prepared on a 50 umol scale. It was purified by Preparative LC/MS 45. The yield of the product was 39 mg, and its estimated purity by LCMS analysis was 98.2%. Analysis condition 8: Retention time=1.49 min. ESI-MS(+) m/z [M+H]⁺: 1832.1.

### Preparation of Example 61

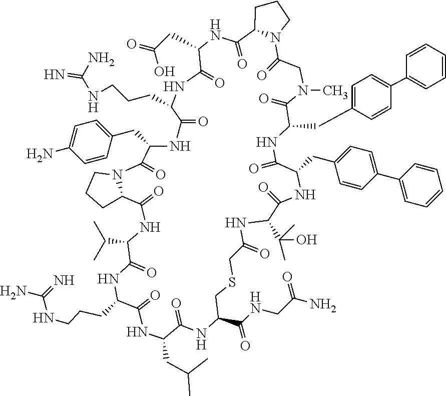 [SMILES: CC(C)C[C@@H]1NC(=O)[C@H](CCCNC(=N)N)NC(=O)[C@H](C(C)C)NC(=O)[C@@H]2CCCN2C(=O)[C@H](Cc2ccc(N)cc2)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](C(C)(C)O)NC(=O)CSC[C@@H](C(=O)NCC(N)=O)NC1=O]

Example 61 was prepared on a 50 umol scale. It was purified by Preparative LC/MS. The yield of the product was 33.4 mg, and its estimated purity by LCMS analysis was 95.8%. Analysis condition 2: Retention time=1.47 min. ESI-MS(+) m/z [M+H]⁺: 1846.1.

### Preparation of Example 62

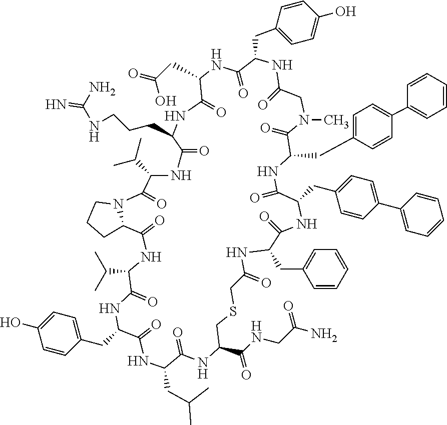 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](Cc2ccccc2)NC(=O)CSC[C@@H](C(=O)NCC(N)=O)NC1=O]

Example 62 was prepared on a 50 umol scale. It was purified by Preparative LC/MS. The yield of the product was 39.5 mg, and its estimated purity by LCMS analysis was 99.3%. Analysis condition 1: Retention time=1.6 min. ESI-MS(+) m/z [M+H]⁺: 1888.1.

### Preparation of Example 63

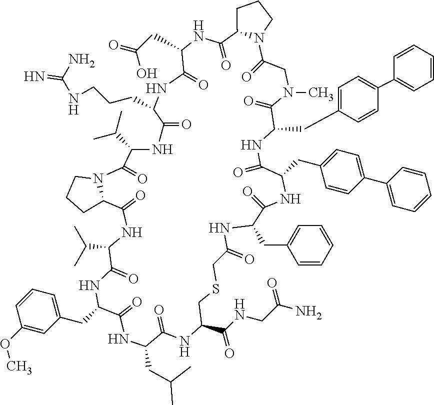 [SMILES: COc1cccc(C[C@@H]2NC(=O)[C@H](C(C)C)NC(=O)[C@@H]3CCCN3C(=O)[C@H](C(C)C)NC(=O)[C@H](CCCNC(=N)N)NC(=O)[C@H](CC(=O)O)NC(=O)[C@@H]3CCCN3C(=O)CN(C)C(=O)[C@H](Cc3ccc(-c4ccccc4)cc3)NC(=O)[C@H](Cc3ccc(-c4ccccc4)cc3)NC(=O)[C@H](Cc3ccccc3)NC(=O)CSC[C@@H](C(=O)NCC(N)=O)NC(=O)[C@H](CC(C)C)NC2=O)c1]

Example 63 was prepared on a 50 umol scale. It was purified by Preparative LC/MS. The yield of the product was 23.5 mg, and its estimated purity by LCMS analysis was 100%. Analysis condition 2: Retention time=1.84 min. ESI-MS(+) m/z [M+H]⁺: 1836.8.

### Preparation of Example 64

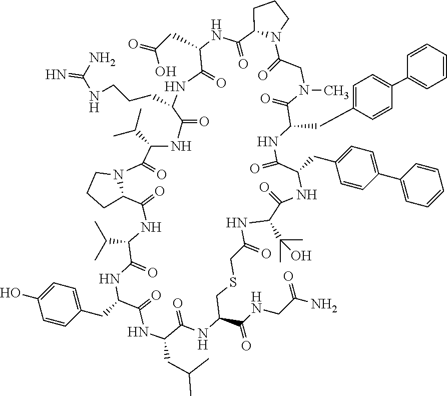 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](C(C)(C)O)NC(=O)CSC[C@@H](C(=O)NCC(N)=O)NC1=O]

Example 64 was prepared on a 0.1 umol scale. It was purified by Preparative LC/MS. The yield of the product was 18.7 mg, and its estimated purity by LCMS analysis was 99.1%. Analysis condition 1: Retention time=1.66 min. ESI-MS(+) m/z [M+H]⁺: 1791.1.

### Preparation of Example 65

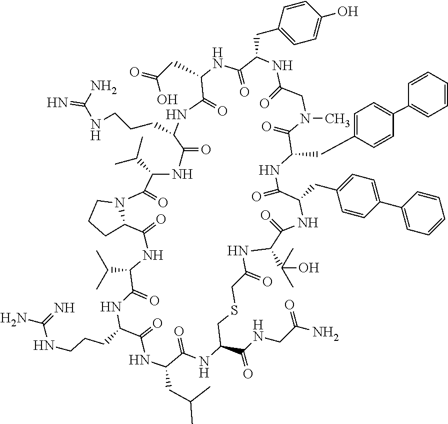 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](C(C)(C)O)NC(=O)CSC[C@@H](C(=O)NCC(N)=O)NC1=O]

Example 65 was prepared on a 50 umol scale. It was purified by Preparative LC/MS 49. The yield of the product was 20.8 mg, and its estimated purity by LCMS analysis was 95.6%. Analysis condition 7: Retention time=1.64 min. ESI-MS(+) m/z [M+H]⁺: 1848.9.

### Preparation of Example 66

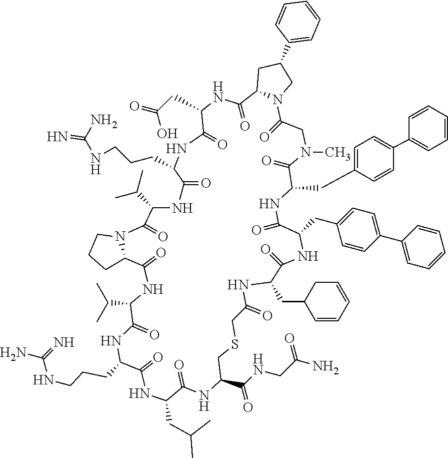 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2C[C@H](c3ccccc3)CN2C(=O)CN(C)C(=O)[C@H](Cc2ccc(-c3ccccc3)cc2)NC(=O)[C@H](Cc2ccc(-c3ccccc3)cc2)NC(=O)[C@H](CC2C=CC=CC2)NC(=O)CSC[C@@H](C(=O)NCC(N)=O)NC1=O]

Example 66 was prepared on a 50 umol scale. It was purified by Preparative LC/MS. The yield of the product was 20.8 mg, and its estimated purity by LCMS analysis was 91.9%. Analysis condition 1: Retention time=1.69 min. ESI-MS(+) m/z [M+H]⁺: 1891.1.

### Preparation of Example 67

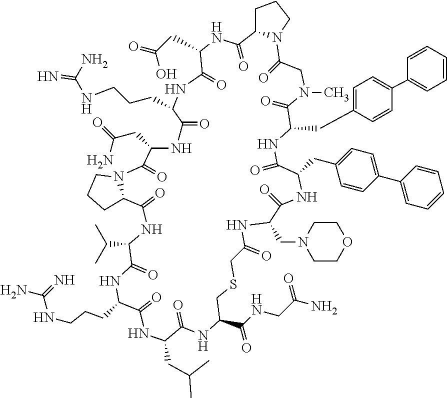 [SMILES: CC(C)C[C@@H]1NC(=O)[C@H](CCCNC(=N)N)NC(=O)[C@H](C(C)C)NC(=O)[C@@H]2CCCN2C(=O)[C@H](CC(N)=O)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 67 was prepared on a 50 umol scale. It was purified by Preparative LC/MS. The yield of the product was 27.4 mg, and its estimated purity by LCMS analysis was 94.1%. Analysis condition 1: Retention time=1.71 min. ESI-MS(+) m/z [M+2H] 2+: 920.2.

### Preparation of Example 68

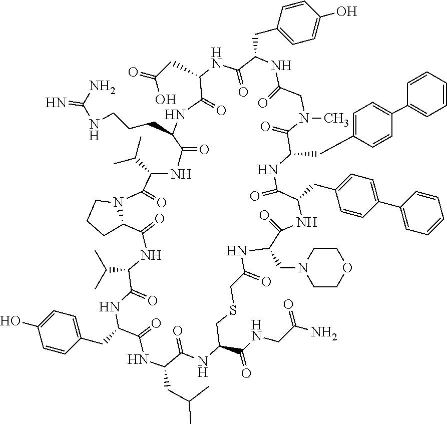 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 68 was prepared on a 50 umol scale. It was purified by Preparative LC/MS. The yield of the product was 14.1 mg, and its estimated purity by LCMS analysis was 97.8%. Analysis condition 1: Retention time=1.67 min. ESI-MS(+) m/z [M+H]⁺: 1897.1.

### Preparation of Example 69

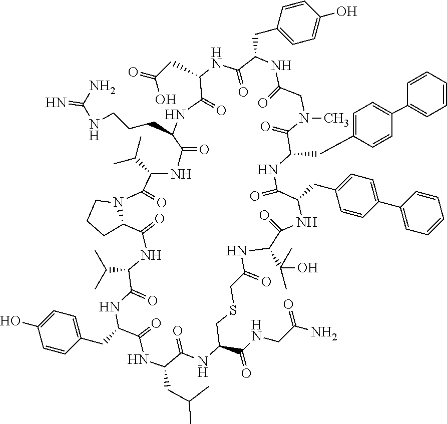 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](C(C)(C)O)NC(=O)CSC[C@@H](C(=O)NCC(N)=O)NC1=O]

Example 69 was prepared on a 50 umol scale. It was purified by Preparative LC/MS. The yield of the product was 17.3 mg, and its estimated purity by LCMS analysis was 96.4%. Analysis condition 2: Retention time=1.7 min. ESI-MS(+) m/z [M+H]⁺: 1856.2.

### Preparation of Example 70

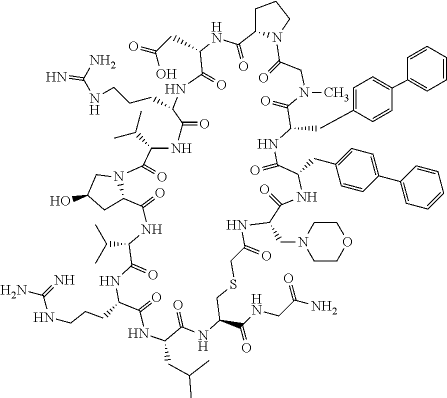 [SMILES: CC(C)C[C@@H]1NC(=O)[C@H](CCCNC(=N)N)NC(=O)[C@H](C(C)C)NC(=O)[C@@H]2C[C@@H](O)CN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 70 was prepared on a 50 umol scale. It was purified by Preparative LC/MS 2. The yield of the product was 7.1 mg, and its estimated purity by LCMS analysis was 96.9%. Analysis condition 8: Retention time=1.7 min. ESI-MS(+) m/z [M+2H] 2+: 921.

### Preparation of Example 71

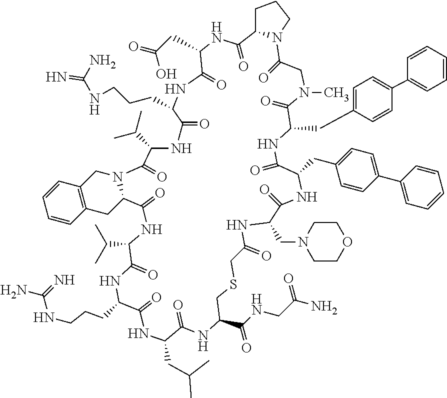 [SMILES: CC(C)C[C@@H]1NC(=O)[C@H](CCCNC(=N)N)NC(=O)[C@H](C(C)C)NC(=O)[C@@H]2Cc3ccccc3CN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 71 was prepared on a 50 umol scale. It was purified by Preparative LC/MS. The yield of the product was 15.6 mg, and its estimated purity by LCMS analysis was 97.4%. Analysis condition 1: Retention time=1.89 min. ESI-MS(+) m/z [M+2H] 2+: 944.

### Preparation of Example 72

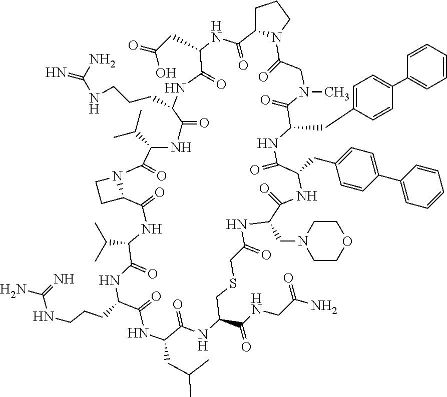 [SMILES: CC(C)C[C@@H]1NC(=O)[C@H](CCCNC(=N)N)NC(=O)[C@H](C(C)C)NC(=O)[C@@H]2CCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 72 was prepared on a 50 umol scale. It was purified by Preparative LC/MS. The yield of the product was 19.5 mg, and its estimated purity by LCMS analysis was 97.5%. Analysis condition 1: Retention time=1.75 min. ESI-MS(+) m/z [M+2H] 2+: 906.4.

### Preparation of Example 73

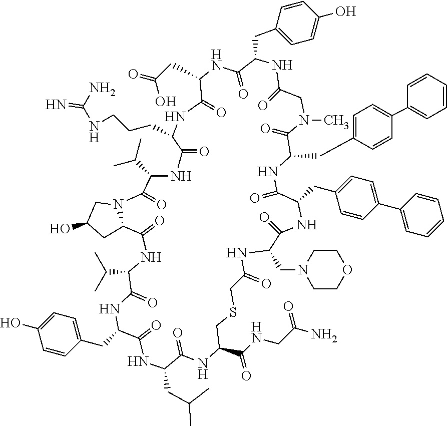 [SMILES: CC(C)C[C@@H]1NC(=O)[C@H](Cc2ccc(O)cc2)NC(=O)[C@H](C(C)C)NC(=O)[C@@H]2C[C@@H](O)CN2C(=O)[C@H](C(C)C)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 73 was prepared on a 50 umol scale. It was purified by Preparative LC/MS. The yield of the product was 19.5 mg, and its estimated purity by LCMS analysis was 94.4%. Analysis condition 1: Retention time=1.67 min. ESI-MS(+) m/z [M+H]⁺: 1913.1.

### Preparation of Example 74

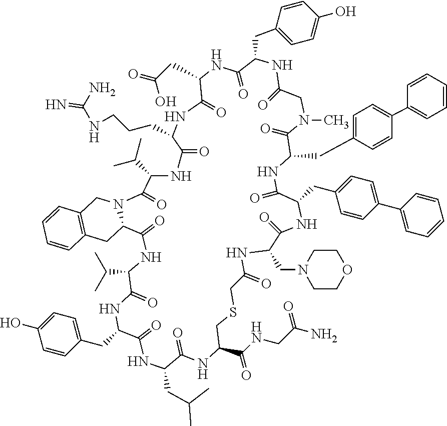 [SMILES: CC(C)C[C@@H]1NC(=O)[C@H](Cc2ccc(O)cc2)NC(=O)[C@H](C(C)C)NC(=O)[C@@H]2Cc3ccccc3CN2C(=O)[C@H](C(C)C)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 74 was prepared on a 50 umol scale. It was purified by Preparative LC/MS. The yield of the product was 10.3 mg, and its estimated purity by LCMS analysis was 98.6%. Analysis condition 1: Retention time=1.89 min. ESI-MS(+) m/z [M+2H] 2+: 980.2.

### Preparation of Example 75

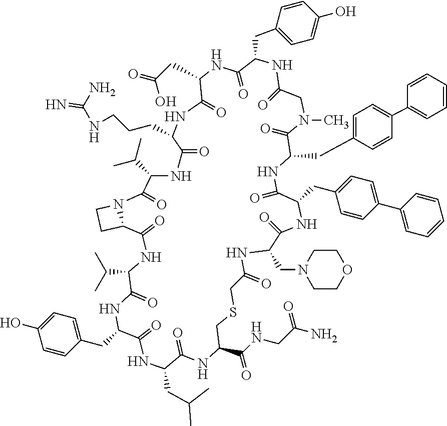 [SMILES: CC(C)C[C@@H]1NC(=O)[C@H](Cc2ccc(O)cc2)NC(=O)[C@H](C(C)C)NC(=O)[C@@H]2CCN2C(=O)[C@H](C(C)C)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 75 was prepared on a 50 umol scale. It was purified by Preparative LC/MS. The yield of the product was 13.1 mg, and its estimated purity by LCMS analysis was 98.8%. Analysis condition 1: Retention time=1.83 min. ESI-MS(+) m/z [M+H]⁺: 1882.9.

### Preparation of Example 76

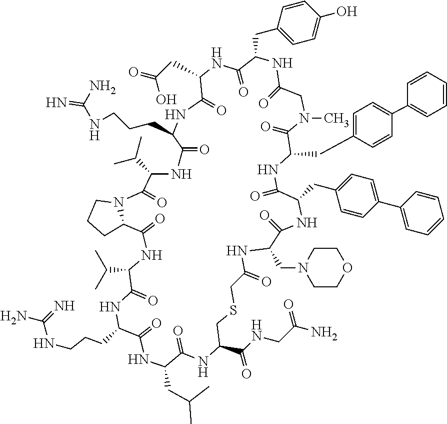 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 76 was prepared on a 50 umol scale. It was purified by Preparative LC/MS. The yield of the product was 19 mg, and its estimated purity by LCMS analysis was 98.6%. Analysis condition 1: Retention time=1.65 min. ESI-MS(+) m/z [M+H]⁺: 1890.1.

### Preparation of Example 77

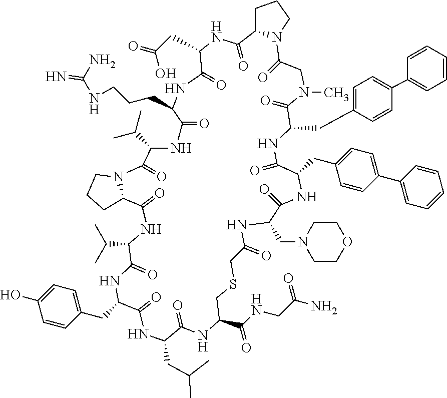 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 77 was prepared on a 50 umol scale. It was purified by Preparative LC/MS. The yield of the product was 17.8 mg, and its estimated purity by LCMS analysis was 97.5%. Analysis condition 2: Retention time=1.56 min. ESI-MS(+) m/z [M+H]⁺: 1831.2.

### Preparation of Example 78

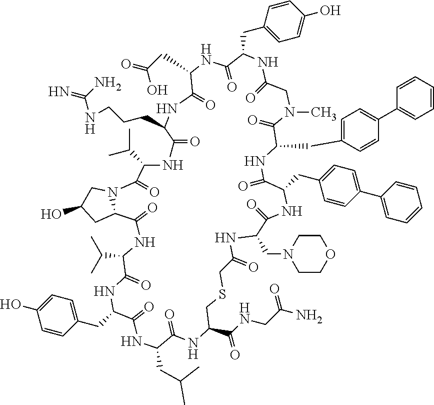 [SMILES: CC(C)C[C@@H]1NC(=O)[C@H](Cc2ccc(O)cc2)NC(=O)[C@H](C(C)C)NC(=O)[C@@H]2C[C@@H](O)CN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 78 was prepared on a 50 umol scale. It was purified by Preparative LC/MS. The yield of the product was 29.4 mg, and its estimated purity by LCMS analysis was 97.3%. Analysis condition 1: Retention time=1.7 min. ESI-MS(+) m/z [M+H]⁺: 1913.1.

### Preparation of Example 79

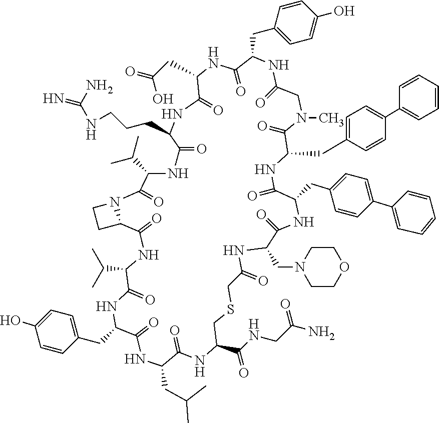 [SMILES: CC(C)C[C@@H]1NC(=O)[C@H](Cc2ccc(O)cc2)NC(=O)[C@H](C(C)C)NC(=O)[C@@H]2CCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 79 was prepared on a 50 umol scale. It was purified by Preparative LC/MS. The yield of the product was 22.2 mg, and its estimated purity by LCMS analysis was 99.4%. Analysis condition 1: Retention time=1.75 min. ESI-MS(+) m/z [M+H]⁺: 1884.2.

### Preparation of Example 80

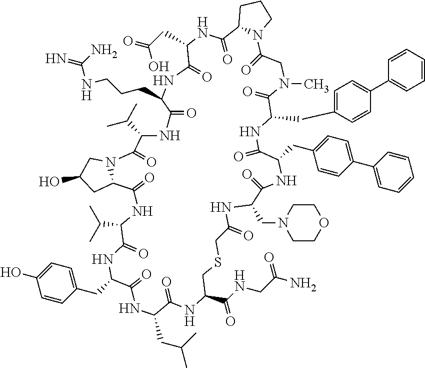 [SMILES: CC(C)C[C@@H]1NC(=O)[C@H](Cc2ccc(O)cc2)NC(=O)[C@H](C(C)C)NC(=O)[C@@H]2C[C@@H](O)CN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 80 was prepared on a 50 umol scale. It was purified by Preparative LC/MS. The yield of the product was 18.6 mg, and its estimated purity by LCMS analysis was 96.3%. Analysis condition 1: Retention time=1.66 min. ESI-MS(+) m/z [M+H]⁺: 1847.2.

### Preparation of Example 81

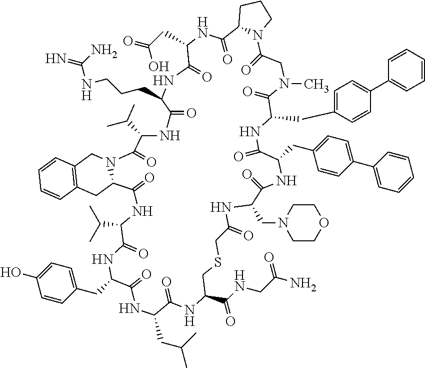 [SMILES: CC(C)C[C@@H]1NC(=O)[C@H](Cc2ccc(O)cc2)NC(=O)[C@H](C(C)C)NC(=O)[C@@H]2Cc3ccccc3CN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 81 was prepared on a 50 umol scale. It was purified by Preparative LC/MS. The yield of the product was 25.3 mg, and its estimated purity by LCMS analysis was 96.3%. Analysis condition 2: Retention time=1.85 min. ESI-MS(+) m/z [M+2H] 2+: 947.2.

### Preparation of Example 82

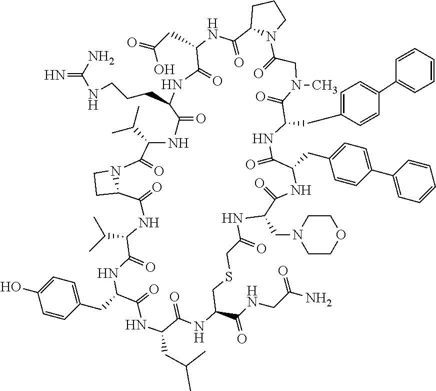 [SMILES: CC(C)C[C@@H]1NC(=O)[C@H](Cc2ccc(O)cc2)NC(=O)[C@H](C(C)C)NC(=O)[C@@H]2CCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 82 was prepared on a 50 umol scale. It was purified by Preparative LC/MS. The yield of the product was 27.5 mg, and its estimated purity by LCMS analysis was 92.2%. Analysis condition 1: Retention time=1.83 min. ESI-MS(+) m/z [M+2H] 2+: 909.2.

### Preparation of Example 83

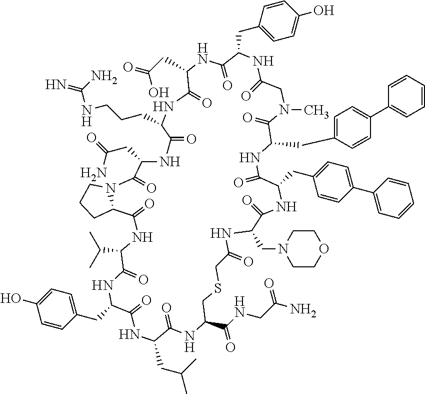 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](CC(N)=O)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 83 was prepared on a 50 umol scale. It was purified by Preparative LC/MS. The yield of the product was 6.7 mg, and its estimated purity by LCMS analysis was 94.8%. Analysis condition 2: Retention time=1.64 min. ESI-MS(+) m/z [M+H]⁺: 1912.2.

### Preparation of Example 84

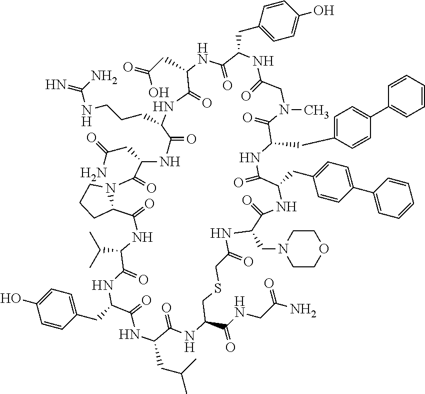 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](CC(N)=O)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 84 was prepared on a 50 umol scale. It was purified by Preparative LC/MS. The yield of the product was 28.1 mg, and its estimated purity by LCMS analysis was 97.1%. Analysis condition 1: Retention time=1.59 min. ESI-MS(+) m/z [M+H]⁺: 1912.1.

### Preparation of Example 85

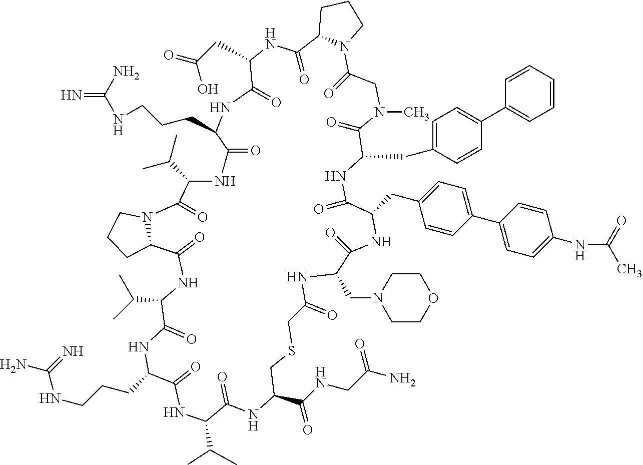 [SMILES: CC(=O)Nc1ccc(-c2ccc(C[C@@H]3NC(=O)[C@H](CN4CCOCC4)NC(=O)CSC[C@@H](C(=O)NCC(N)=O)NC(=O)[C@H](C(C)C)NC(=O)[C@H](CCCNC(=N)N)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C(=O)[C@H](Cc4ccc(-c5ccccc5)cc4)NC3=O)cc2)cc1]

Example 85 was prepared on a 50 umol scale. It was purified by Preparative LC/MS. The yield of the product was 14 mg, and its estimated purity by LCMS analysis was 86.8%. Analysis condition 2: Retention time=1.43 min. ESI-MS(+) m/z [M+2H] 2+: 934.2.

### Preparation of Example 86

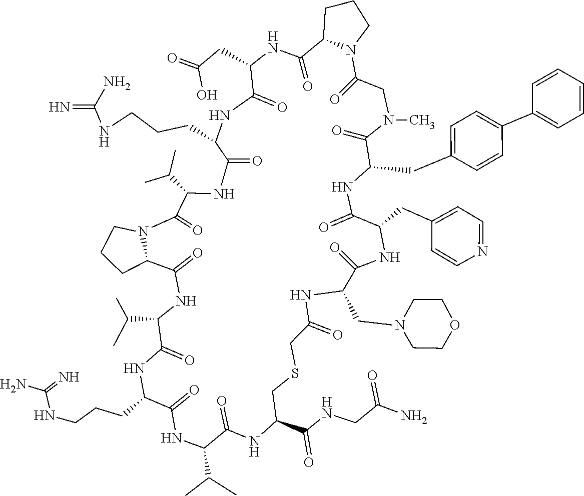 [SMILES: CC(C)[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccncc2)NC(=O)[C@H](CN2CCOCC2)NC(=O)CSC[C@@H](C(=O)NCC(N)=O)NC1=O]

Example 86 was prepared on a 50 umol scale. It was purified by Preparative LC/MS. The yield of the product was 3.4 mg, and its estimated purity by LCMS analysis was 98.9%. Analysis condition 1: Retention time=1.38 min. ESI-MS(+) m/z [M+H]⁺: 1734.89.

### Preparation of Example 87

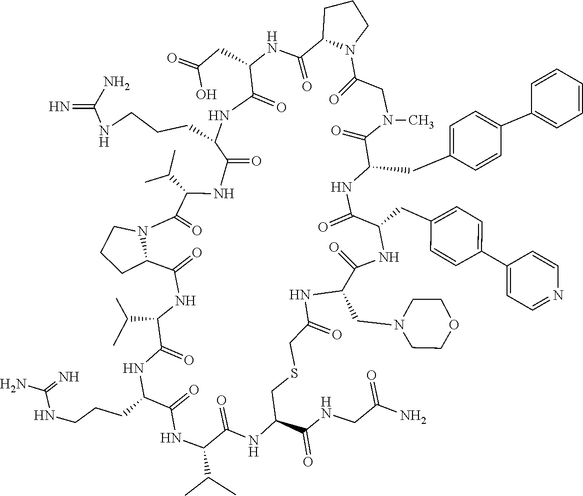 [SMILES: CC(C)[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccncc3)cc2)NC(=O)[C@H](CN2CCOCC2)NC(=O)CSC[C@@H](C(=O)NCC(N)=O)NC1=O]

Example 87 was prepared on a 50 umol scale. It was purified by Preparative LC/MS. The yield of the product was 8.6 mg, and its estimated purity by LCMS analysis was 91.5%. Analysis condition 1: Retention time=1.27 min. ESI-MS(+) m/z [M+2H] 2+: 906.33.

### Preparation of Example 88

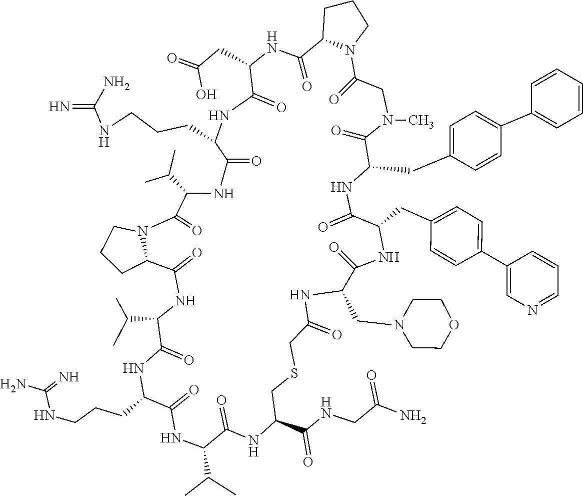 [SMILES: CC(C)[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3cccnc3)cc2)NC(=O)[C@H](CN2CCOCC2)NC(=O)CSC[C@@H](C(=O)NCC(N)=O)NC1=O]

Example 88 was prepared on a 50 umol scale. It was purified by Preparative LC/MS. The yield of the product was 12.4 mg, and its estimated purity by LCMS analysis was 88%. Analysis condition 1: Retention time=1.28 min. ESI-MS(+) m/z [M+2H] 2+: 906.4.

### Preparation of Example 89

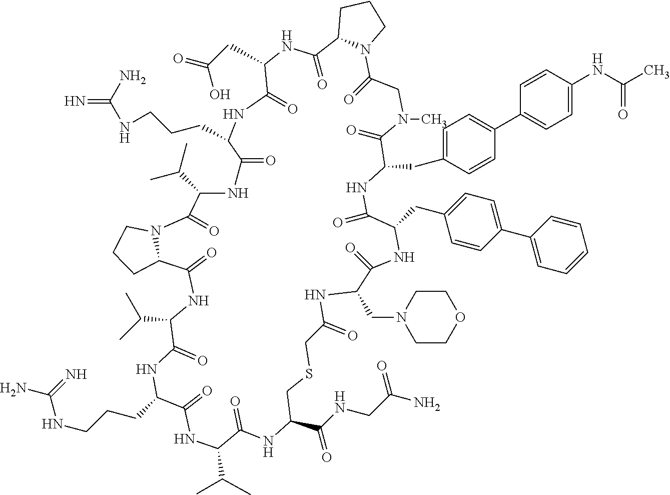 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](C(C)C)NC(=O)[C@H](CCCNC(=N)N)NC(=O)[C@H](C(C)C)NC(=O)[C@@H]4CCCN4C(=O)[C@H](C(C)C)NC(=O)[C@H](CCCNC(=N)N)NC(=O)[C@H](CC(=O)O)NC(=O)[C@@H]4CCCN4C(=O)CN(C)C3=O)cc2)cc1]

Example 89 was prepared on a 50 umol scale. It was purified by Preparative LC/MS. The yield of the product was 8 mg, and its estimated purity by LCMS analysis was 96.8%. Analysis condition 1: Retention time=1.54 min. ESI-MS(+) m/z [M+H]⁺: 1867.

### Preparation of Example 90

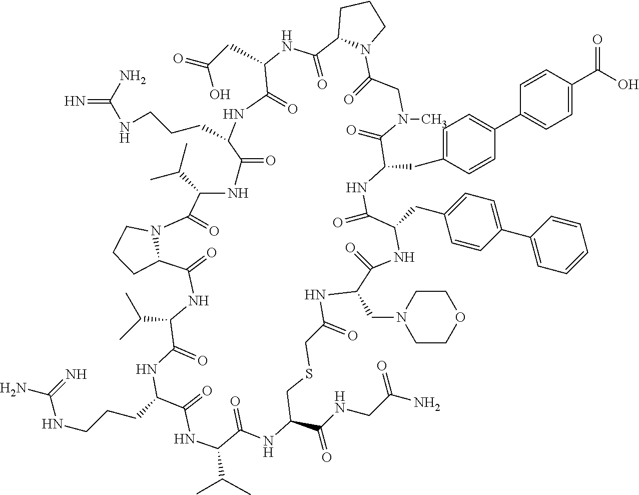 [SMILES: CC(C)[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccc(C(=O)O)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 90 was prepared on a 50 umol scale. It was purified by Preparative LC/MS. The yield of the product was 10.9 mg, and its estimated purity by LCMS analysis was 85.7%. Analysis condition 1: Retention time=1.36 min. ESI-MS(+) m/z [M+H]⁺: 1854.1.

### Preparation of Example 91

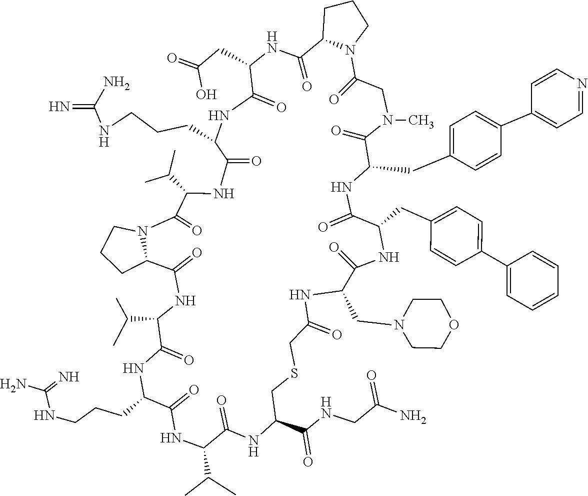 [SMILES: CC(C)[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccncc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 91 was prepared on a 50 umol scale. It was purified by Preparative LC/MS. The yield of the product was 9.6 mg, and its estimated purity by LCMS analysis was 88.8%. Analysis condition 2: Retention time=1.3 min. ESI-MS(+) m/z [M+H]⁺: 1810.8.

### Preparation of Example 92

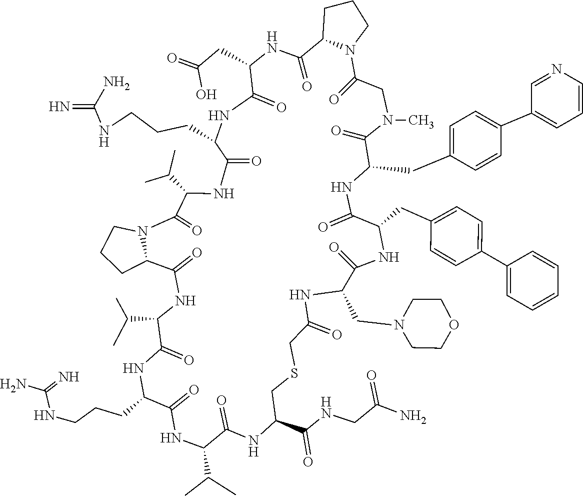 [SMILES: CC(C)[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3cccnc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 92 was prepared on a 50 umol scale. It was purified by Preparative LC/MS. The yield of the product was 7.3 mg, and its estimated purity by LCMS analysis was 88.8%. Analysis condition 2: Retention time=1.32 min. ESI-MS(+) m/z [M+H]⁺: 1811.1.

### Preparation of Example 93

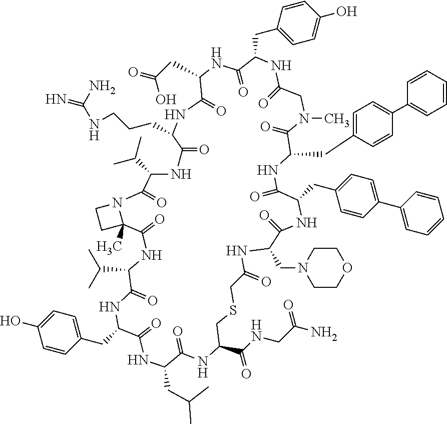 [SMILES: CC(C)C[C@@H]1NC(=O)[C@H](Cc2ccc(O)cc2)NC(=O)[C@H](C(C)C)NC(=O)[C@]2(C)CCN2C(=O)[C@H](C(C)C)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 93 was prepared on a 50 umol scale. It was purified by Preparative LC/MS. The yield of the product was 7.2 mg, and its estimated purity by LCMS analysis was 97.8%. Analysis condition 1: Retention time=1.82 min. ESI-MS(+) m/z [M+H]⁺: 1895.2.

### Preparation of Example 94

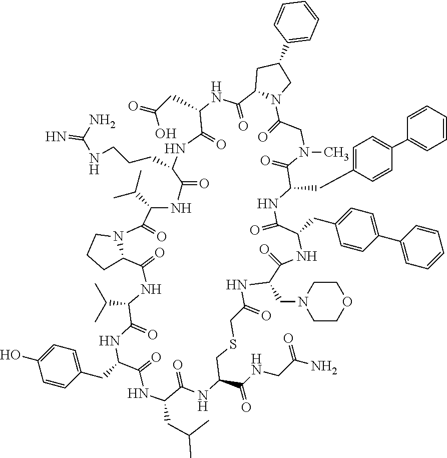 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2C[C@H](c3ccccc3)CN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 94 was prepared on a 50 umol scale. It was purified by Preparative LC/MS. The yield of the product was 14.7 mg, and its estimated purity by LCMS analysis was 95%. Analysis condition 1: Retention time=1.83 min. ESI-MS(+) m/z [M+H]⁺: 1907.

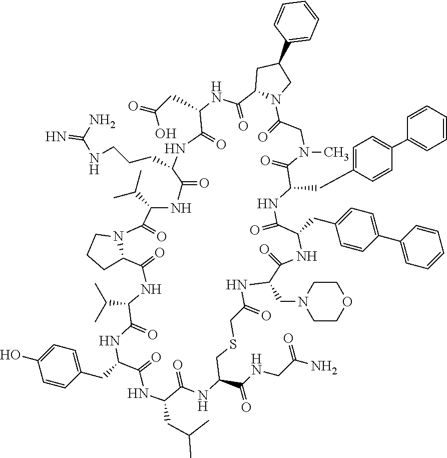 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2C[C@@H](c3ccccc3)CN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 95 was prepared on a 50 umol scale. It was purified by Preparative LC/MS. The yield of the product was 34.8 mg, and its estimated purity by LCMS analysis was 95.1%. Analysis condition 1: Retention time=1.94 min. ESI-MS(+) m/z [M+H]⁺: 1907.2.

### Preparation of Example 96

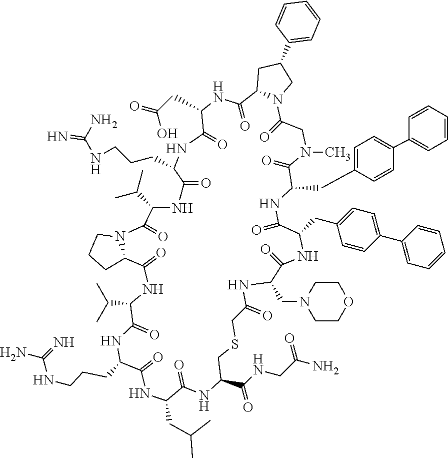 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2C[C@H](c3ccccc3)CN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 96 was prepared on a 50 umol scale. It was purified by Preparative LC/MS. The yield of the product was 21.4 mg, and its estimated purity by LCMS analysis was 95.2%. Analysis condition 2: Retention time=1.79 min. ESI-MS(+) m/z [M+H]⁺: 1900.1.

### Preparation of Example 97

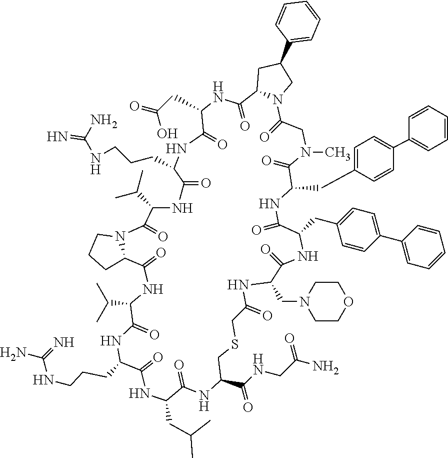 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2C[C@@H](c3ccccc3)CN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 97 was prepared on a 50 umol scale. It was purified by Preparative LC/MS. The yield of the product was 6.3 mg, and its estimated purity by LCMS analysis was 97.5%. Analysis condition 2: Retention time=1.71 min. ESI-MS(+) m/z [M+H]⁺: 1900.1.

### Preparation of Example 98

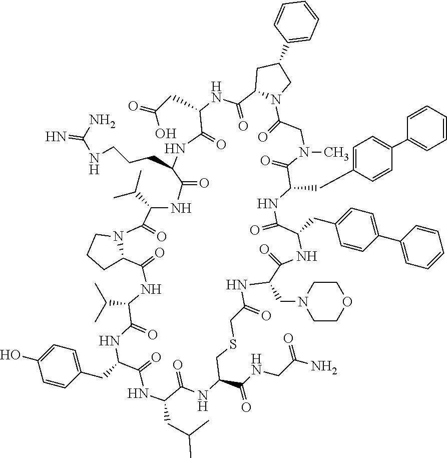 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2C[C@H](c3ccccc3)CN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 98 was prepared on a 50 umol scale. It was purified by Preparative LC/MS. The yield of the product was 24.6 mg, and its estimated purity by LCMS analysis was 99.1%. Analysis condition 2: Retention time=1.67 min. ESI-MS(+) m/z [M+H]⁺: 1908.2.

### Preparation of Example 99

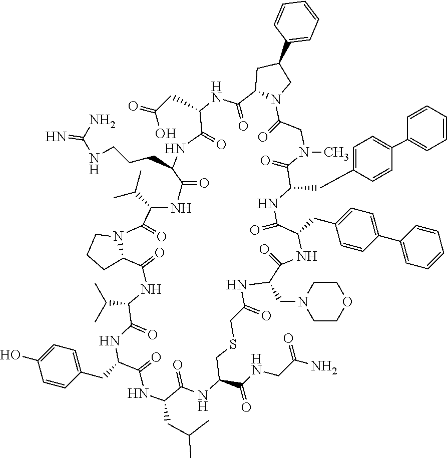 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2C[C@@H](c3ccccc3)CN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 99 was prepared on a 50 umol scale. It was purified by Preparative LC/MS. The yield of the product was 24.1 mg, and its estimated purity by LCMS analysis was 98.9%. Analysis condition 1: Retention time=1.44 min. ESI-MS(+) m/z [M+H]⁺: 1907.

### Preparation of Example 100

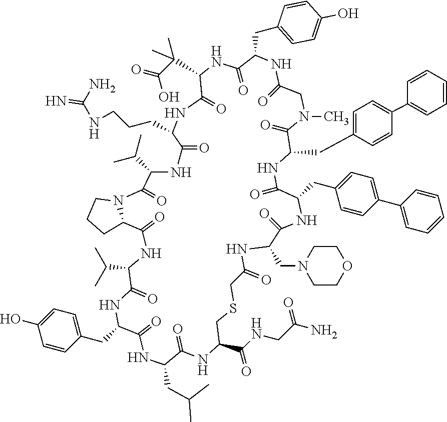 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@H](CCCNC(=N)N)NC(=O)[C@H](C(C)(C)C(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 100 was prepared on a 50 umol scale. It was purified by Preparative LC/MS. The yield of the product was 13.4 mg, and its estimated purity by LCMS analysis was 100%. Analysis condition 2: Retention time=1.76 min. ESI-MS(+) m/z [M+H]⁺: 1925.

### Preparation of Example 101

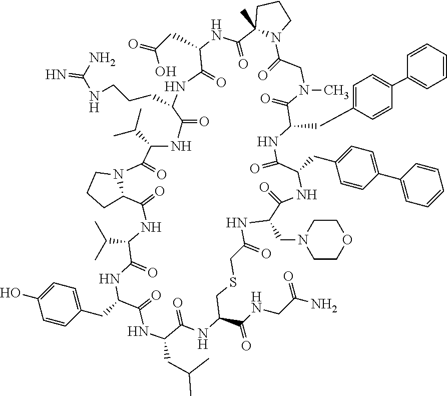 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@]2(C)CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 101 was prepared on a 50 umol scale. It was purified by Preparative LC/MS. The yield of the product was 8.3 mg, and its estimated purity by LCMS analysis was 96.7%. Analysis condition 2: Retention time=1.8 min. ESI-MS(+) m/z [M+H]⁺: 1845.2.

### Preparation of Example 102

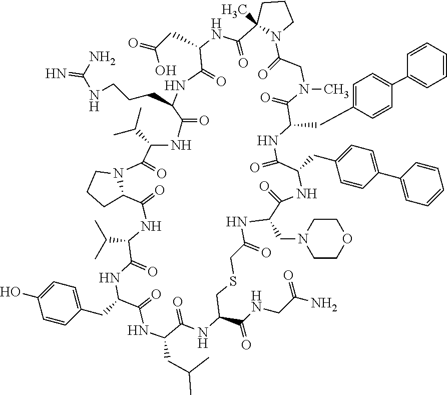 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@]2(C)CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 102 was prepared on a 50 umol scale. It was purified by Preparative LC/MS. The yield of the product was 24 mg, and its estimated purity by LCMS analysis was 94.7%. Analysis condition 1: Retention time=1.32 min. ESI-MS(+) m/z [M+2H] 2+: 923.2.

### Preparation of Example 103

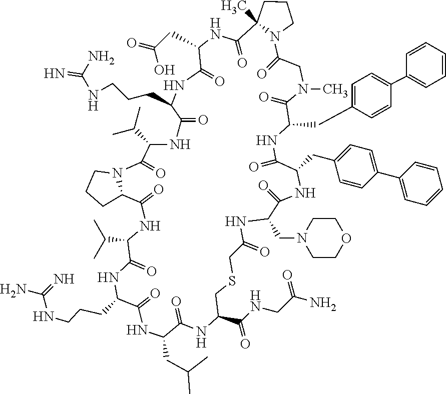 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@]2(C)CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 103 was prepared on a 50 umol scale. It was purified by Preparative LC/MS. The yield of the product was 17.8 mg, and its estimated purity by LCMS analysis was 95.7%. Analysis condition 2: Retention time=1.58 min. ESI-MS(+) m/z [M+2H] 2+: 919.2.

### Preparation of Example 104

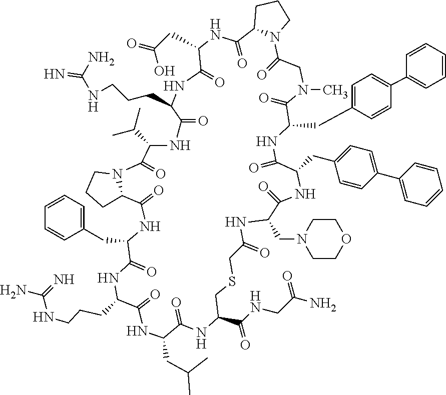 [SMILES: CC(C)C[C@@H]1NC(=O)[C@H](CCCNC(=N)N)NC(=O)[C@H](Cc2ccccc2)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 104 was prepared on a 50 umol scale. It was purified by Preparative LC/MS. The yield of the product was 3.1 mg, and its estimated purity by LCMS analysis was 92.1%. Analysis condition 1: Retention time=1.64 min. ESI-MS(+) m/z [M+H]⁺: 1872.2.

### Preparation of Example 105

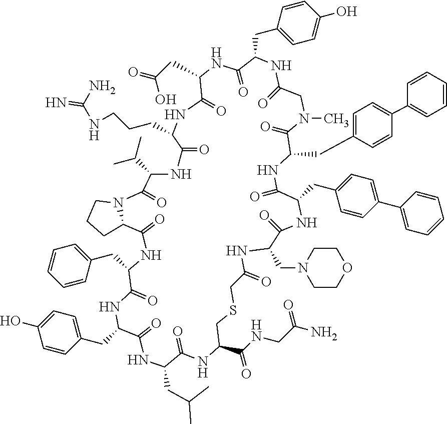 [SMILES: CC(C)C[C@@H]1NC(=O)[C@H](Cc2ccc(O)cc2)NC(=O)[C@H](Cc2ccccc2)NC(=O)[C@@H]2CCCN2C(=O)[C@H](C(C)C)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 105 was prepared on a 50 umol scale. It was purified by Preparative LC/MS. The yield of the product was 7.5 mg, and its estimated purity by LCMS analysis was 91.7%. Analysis condition 1: Retention time=1.9 min. ESI-MS(+) m/z [M+H]⁺: 1932.1.

### Preparation of Example 106

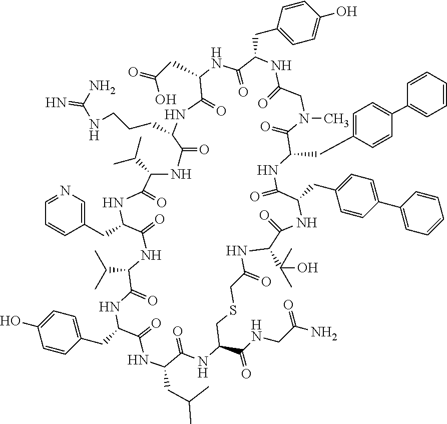 [SMILES: CC(C)C[C@@H]1NC(=O)[C@H](Cc2ccc(O)cc2)NC(=O)[C@H](C(C)C)NC(=O)[C@H](Cc2cccnc2)NC(=O)[C@H](C(C)C)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](C(C)(C)O)NC(=O)CSC[C@@H](C(=O)NCC(N)=O)NC1=O]

Example 106 was prepared on a 50 umol scale. It was purified by Preparative LC/MS. The yield of the product was 15.6 mg, and its estimated purity by LCMS analysis was 98.3%. Analysis condition 1: Retention time=1.72 min. ESI-MS(+) m/z [M+H]⁺: 1908.0.

### Preparation of Example 107

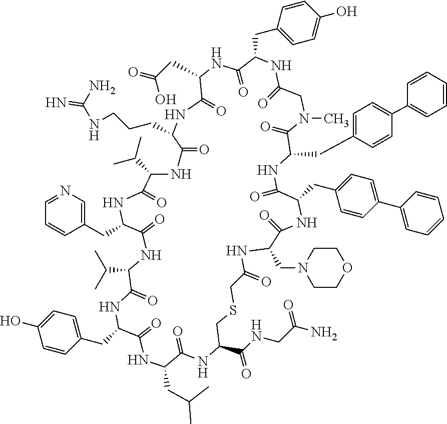 [SMILES: CC(C)C[C@@H]1NC(=O)[C@H](Cc2ccc(O)cc2)NC(=O)[C@H](C(C)C)NC(=O)[C@H](Cc2cccnc2)NC(=O)[C@H](C(C)C)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 107 was prepared on a 50 umol scale. It was purified by Preparative LC/MS. The yield of the product was 26.4 mg, and its estimated purity by LCMS analysis was 94.7%. Analysis condition 1: Retention time=1.73 min. ESI-MS(+) m/z [M+H]⁺: 1948.

### Preparation of Example 108

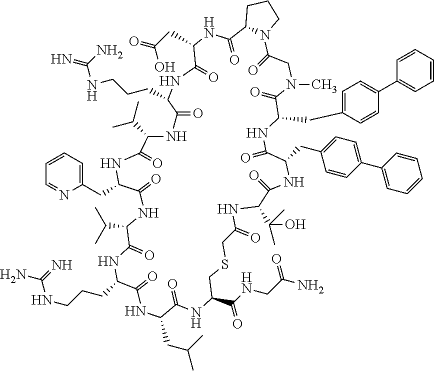 [SMILES: CC(C)C[C@@H]1NC(=O)[C@H](CCCNC(=N)N)NC(=O)[C@H](C(C)C)NC(=O)[C@H](Cc2ccccn2)NC(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](C(C)(C)O)NC(=O)CSC[C@@H](C(=O)NCC(N)=O)NC1=O]

Example 108 was prepared on a 50 umol scale. It was purified by Preparative LC/MS. The yield of the product was 13.1 mg, and its estimated purity by LCMS analysis was 95.4%. Analysis condition 2: Retention time=1.56 min. ESI-MS(+) m/z [M+H]⁺: 1834.1.

### Preparation of Example 109

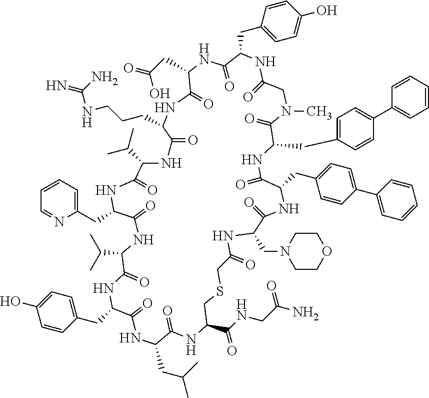 [SMILES: CC(C)C[C@@H]1NC(=O)[C@H](Cc2ccc(O)cc2)NC(=O)[C@H](C(C)C)NC(=O)[C@H](Cc2ccccn2)NC(=O)[C@H](C(C)C)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 109 was prepared on a 50 umol scale. It was purified by Preparative LC/MS. The yield of the product was 16.8 mg, and its estimated purity by LCMS analysis was 94.9%. Analysis condition 1: Retention time=1.74 min. ESI-MS(+) m/z [M+H]⁺: 1948.9.

### Preparation of Example 110

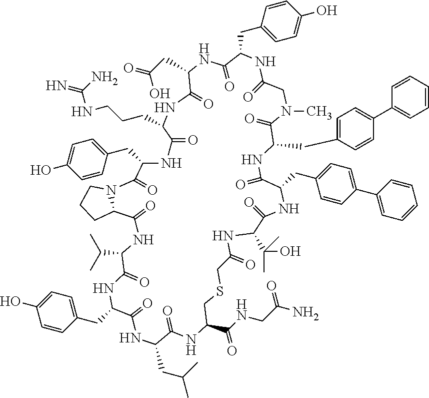 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](Cc2ccc(O)cc2)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](C(C)(C)O)NC(=O)CSC[C@@H](C(=O)NCC(N)=O)NC1=O]

Example 110 was prepared on a 50 umol scale. It was purified by Preparative LC/MS. The yield of the product was 23.4 mg, and its estimated purity by LCMS analysis was 92.6%. Analysis condition 1: Retention time=1.73 min. ESI-MS(+) m/z [M+H]⁺: 1920.1.

### Preparation of Example 111

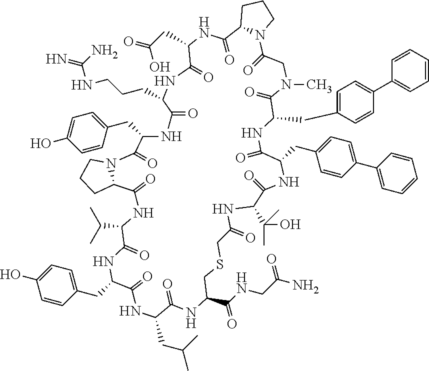 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](Cc2ccc(O)cc2)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](C(C)(C)O)NC(=O)CSC[C@@H](C(=O)NCC(N)=O)NC1=O]

Example 111 was prepared on a 50 umol scale. It was purified by Preparative LC/MS. The yield of the product was 23 mg, and its estimated purity by LCMS analysis was 98.7%. Analysis condition 1: Retention time=1.73 min. ESI-MS(+) m/z [M+H]⁺: 1855.

### Preparation of Example 112

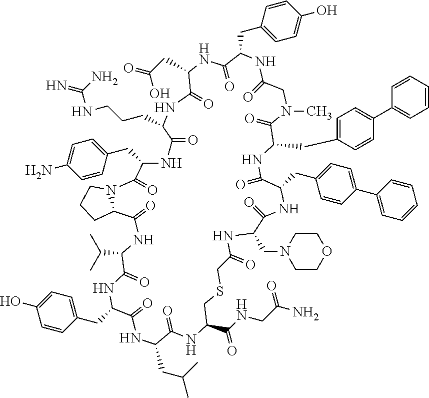 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](Cc2ccc(N)cc2)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 112 was prepared on a 50 umol scale. It was purified by Preparative LC/MS. The yield of the product was 8.9 mg, and its estimated purity by LCMS analysis was 90.3%. Analysis condition 1: Retention time=1.66 min. ESI-MS(+) m/z [M+2H] 2+: 981.2.

### Preparation of Example 113

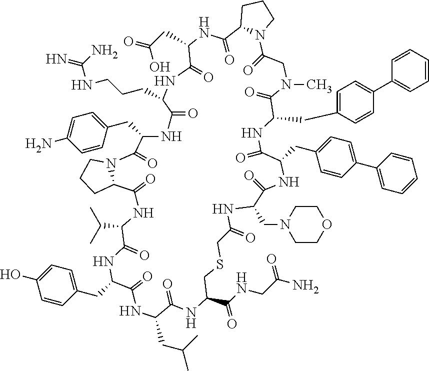 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](Cc2ccc(N)cc2)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 113 was prepared on a 50 umol scale. It was purified by Preparative LC/MS. The yield of the product was 16.5 mg, and its estimated purity by LCMS analysis was 95.4%. Analysis condition 2: Retention time=1.6 min. ESI-MS(+) m/z [M+H]⁺: 1893.2.

### Preparation of Example 114

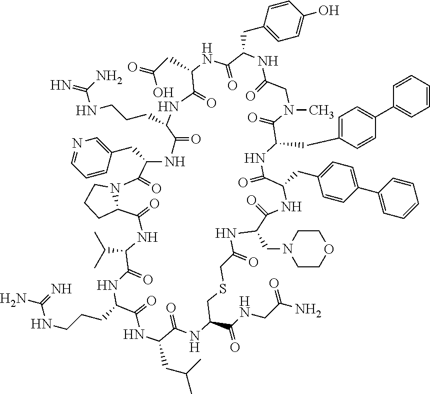 [SMILES: CC(C)C[C@@H]1NC(=O)[C@H](CCCNC(=N)N)NC(=O)[C@H](C(C)C)NC(=O)[C@@H]2CCCN2C(=O)[C@H](Cc2cccnc2)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 114 was prepared on a 50 umol scale. It was purified by Preparative LC/MS. The yield of the product was 20.6 mg, and its estimated purity by LCMS analysis was 94.7%. Analysis condition 1: Retention time=1.67 min. ESI-MS(+) m/z [M+H]⁺: 1940.

### Preparation of Example 115

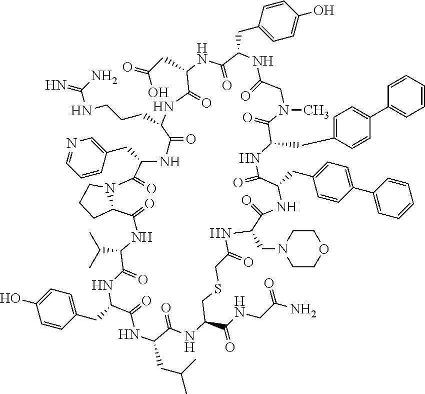 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](Cc2cccnc2)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 115 was prepared on a 50 umol scale. It was purified by Preparative LC/MS. The yield of the product was 26.5 mg, and its estimated purity by LCMS analysis was 93.2%. Analysis condition 1: Retention time=1.67 min. ESI-MS(+) m/z [M+H]⁺: 1946.2.

### Preparation of Example 116

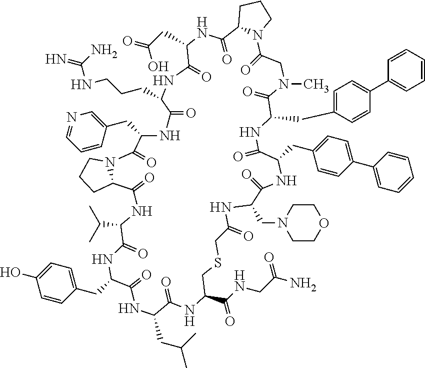 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](Cc2cccnc2)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 116 was prepared on a 50 umol scale. It was purified by Preparative LC/MS. The yield of the product was 15.5 mg, and its estimated purity by LCMS analysis was 95.5%. Analysis condition 1: Retention time=1.65 min. ESI-MS(+) m/z [M+H]⁺: 1881.

### Preparation of Example 117

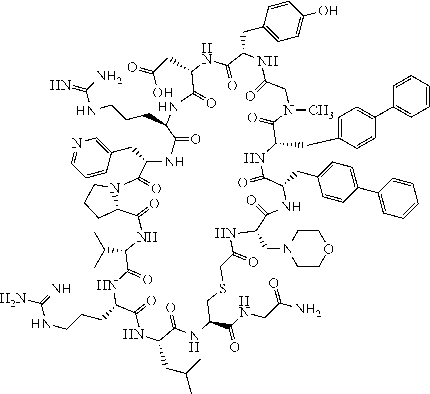 [SMILES: CC(C)C[C@@H]1NC(=O)[C@H](CCCNC(=N)N)NC(=O)[C@H](C(C)C)NC(=O)[C@@H]2CCCN2C(=O)[C@H](Cc2cccnc2)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 117 was prepared on a 50 umol scale. It was purified by Preparative LC/MS. The yield of the product was 17.2 mg, and its estimated purity by LCMS analysis was 94.3%. Analysis condition 1: Retention time=1.61 min. ESI-MS(+) m/z [M+H]⁺: 1940.1.

### Preparation of Example 118

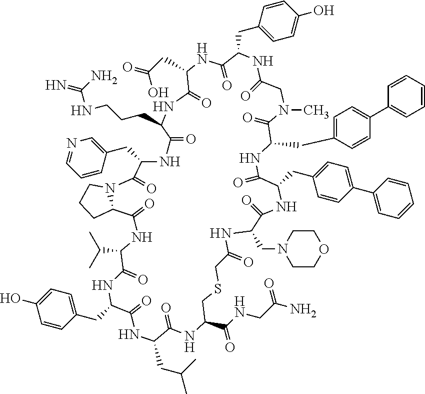 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](Cc2cccnc2)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 118 was prepared on a 50 umol scale. It was purified by Preparative LC/MS. The yield of the product was 19.9 mg, and its estimated purity by LCMS analysis was 98.5%. Analysis condition 1: Retention time=1.61 min. ESI-MS(+) m/z [M+H]⁺: 1946.1.

### Preparation of Example 119

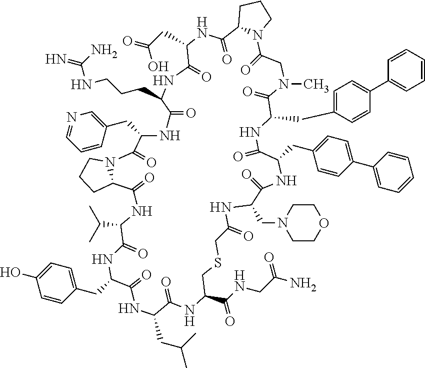 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](Cc2cccnc2)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 119 was prepared on a 50 umol scale. It was purified by Preparative LC/MS. The yield of the product was 20.6 mg, and its estimated purity by LCMS analysis was 98.6%. Analysis condition 2: Retention time=1.47 min. ESI-MS(+) m/z [M+H]⁺: 1878.

### Preparation of Example 120

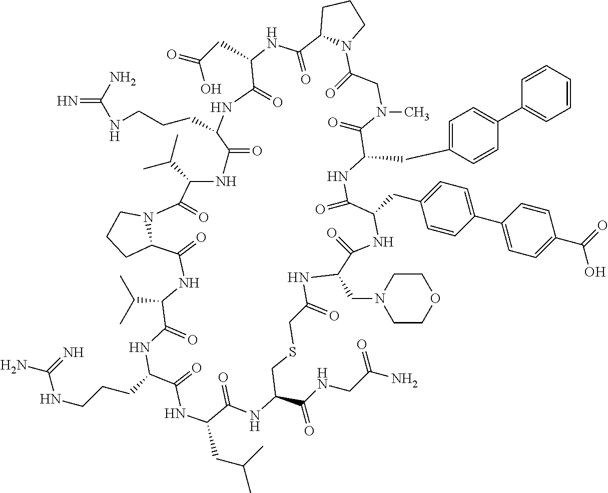 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC1=O]

Example 120 was prepared on a 50 umol scale. It was purified by Preparative LC/MS. The yield of the product was 25.1 mg, and its estimated purity by LCMS analysis was 92.6%. Analysis condition 1: Retention time=1.35 min. ESI-MS(+) m/z [M+H]⁺: 1869.2.

### Preparation of Example 121

### 4′-{[(3S,6S,9S,12R,18S,21S,24S,30S,33S,36S,39S,44aS)-24-({[1,1′-biphenyl]-4-yl}methyl)-36-(3-carbamimidamidopropyl)-12-[(carbamoylmethyl) carbamoyl]-33-(carboxymethyl)-6,30-bis[(4-hydroxyphenyl)methyl]-26-methyl-9-(2-methylpropyl)-18-[(morpholin-4-yl)methyl]-1,4,7,10,16,19,22,25,28,31,34,37,40-tridecaoxo-3,39-bis(propan-2-yl)-dotetracontahydro-1H-pyrrolo[2,1-o]1-thia-4,7,10,13,16,19,22,25,28,31,34,37,40-tridecaazacyclodotetracontan-21-yl]methyl}-[1,1′-biphenyl]-4-carboxylic acid

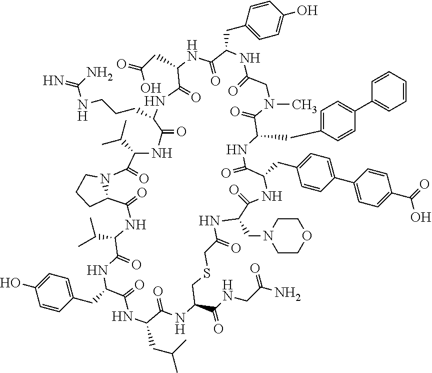 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC1=O]

Example 121 was prepared on a 50 umol scale. It was purified by Preparative LC/MS. The yield of the product was 12.4 mg, and its estimated purity by LCMS analysis was 92.6%. Analysis condition 2: Retention time=1.49 min. ESI-MS(+) m/z [M+H]⁺: 1942.2.

### Preparation of Example 122

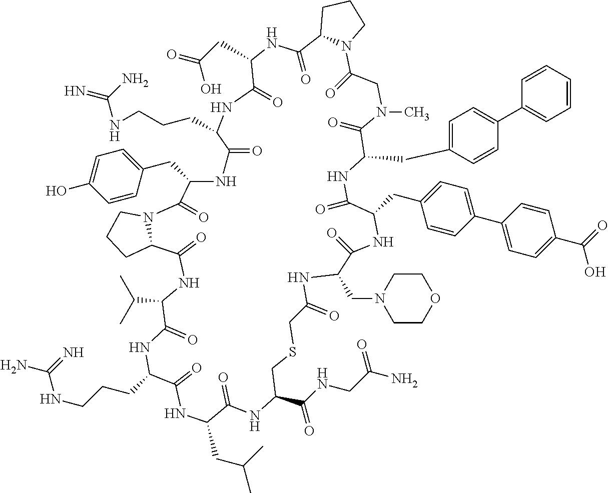 [SMILES: CC(C)C[C@@H]1NC(=O)[C@H](CCCNC(=N)N)NC(=O)[C@H](C(C)C)NC(=O)[C@@H]2CCCN2C(=O)[C@H](Cc2ccc(O)cc2)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC1=O]

Example 122 was prepared on a 50 umol scale. It was purified by Preparative LC/MS. The yield of the product was 13.1 mg, and its estimated purity by LCMS analysis was 91%. Analysis condition 1: Retention time=1.55 min. ESI-MS(+) m/z [M+2H] 2+: 967.2.

### Preparation of Example 123

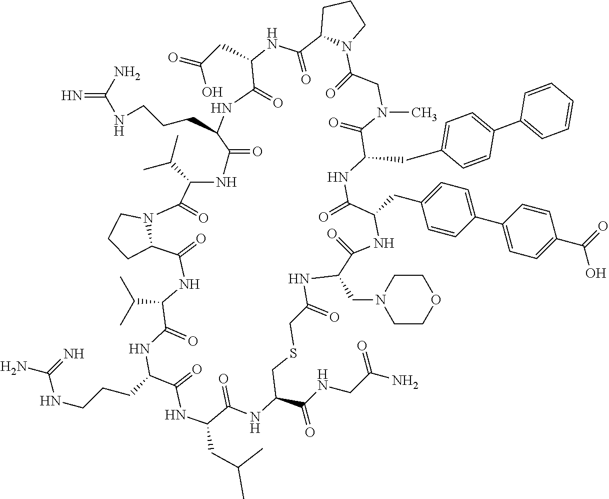 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC1=O]

Example 123 was prepared on a 50 umol scale. It was purified by Preparative LC/MS. The yield of the product was 4.6 mg, and its estimated purity by LCMS analysis was 92.8%. Analysis condition 2: Retention time=1.35 min. ESI-MS(+) m/z [M+2H] 2+: 935.

### Preparation of Example 124

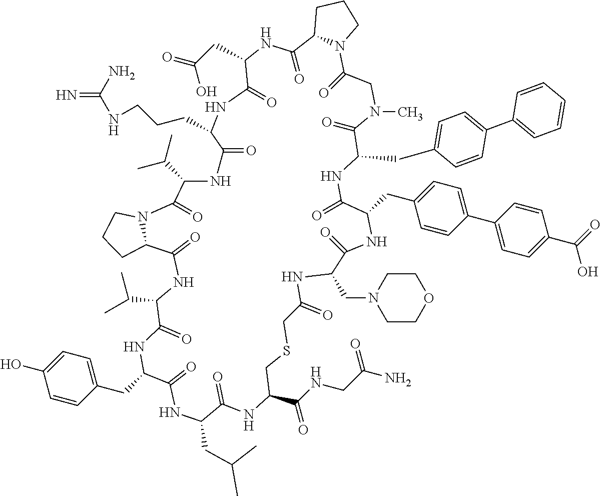 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC1=O]

Example 124 was prepared on a 50 umol scale. It was purified by Preparative LC/MS. The yield of the product was 9.7 mg, and its estimated purity by LCMS analysis was 90.6%. Analysis condition 1: Retention time=1.41 min. ESI-MS(+) m/z [M+H]⁺: 1876.2.

### Preparation of Example 125

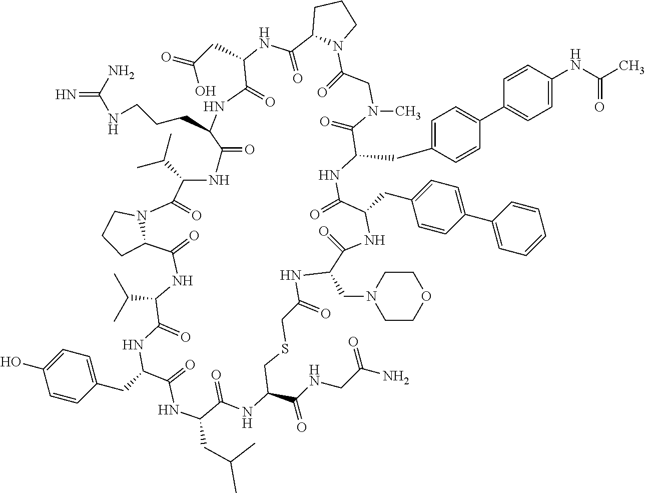 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 125 was prepared on a 50 Preparative LC/MS. The yield of the product was 18.8 mg, and its estimated purity by LCMS analysis was 96.6%. Analysis condition 2: Retention time=1.47 min. ESI-MS(+) m/z [M+H]⁺: 1888.9.

### Preparation of Example 126

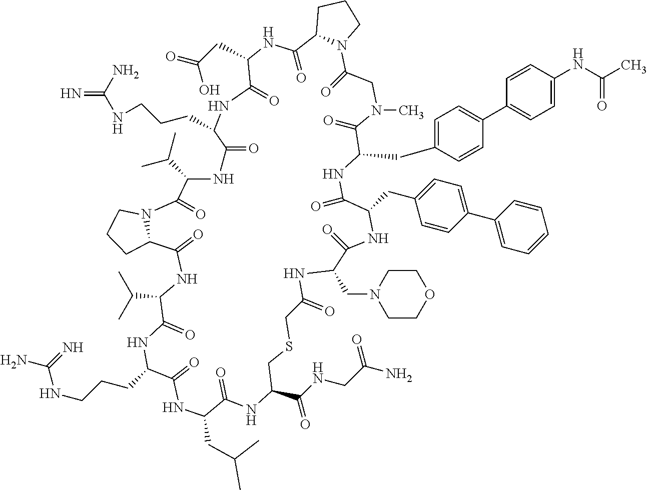 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]4CCCN4C(=O)[C@H](C(C)C)NC(=O)[C@H](CCCNC(=N)N)NC(=O)[C@H](CC(=O)O)NC(=O)[C@@H]4CCCN4C(=O)CN(C)C3=O)cc2)cc1]

Example 126 was prepared on a 50 umol scale. It was purified by Preparative LC/MS. The yield of the product was 11.9 mg, and its estimated purity by LCMS analysis was 90.4%. Analysis condition 2: Retention time=1.4 min. ESI-MS(+) m/z [M+H]⁺: 1881.

### Preparation of Example 127

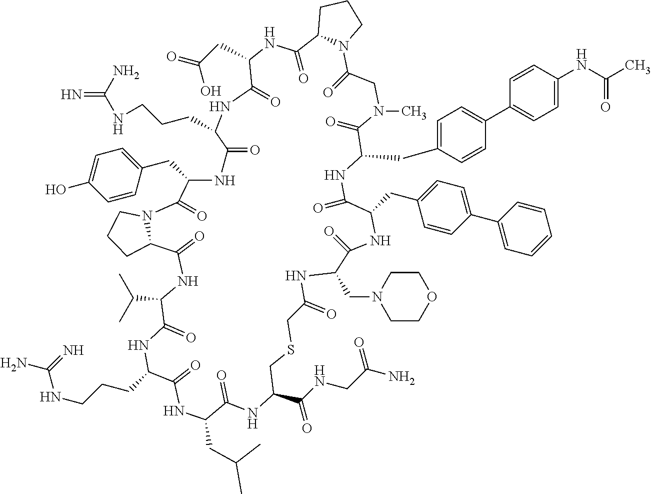 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]4CCCN4C(=O)[C@H](Cc4ccc(O)cc4)NC(=O)[C@H](CCCNC(=N)N)NC(=O)[C@H](CC(=O)O)NC(=O)[C@@H]4CCCN4C(=O)CN(C)C3=O)cc2)cc1]

Example 127 was prepared on a 50 Preparative LC/MS. The yield of the product was 13.6 mg, and its estimated purity by LCMS analysis was 98.2%. Analysis condition 2: Retention time=1.37 min. ESI-MS(+) m/z [M+3H] 3+: 649.2.

### Preparation of Example 128

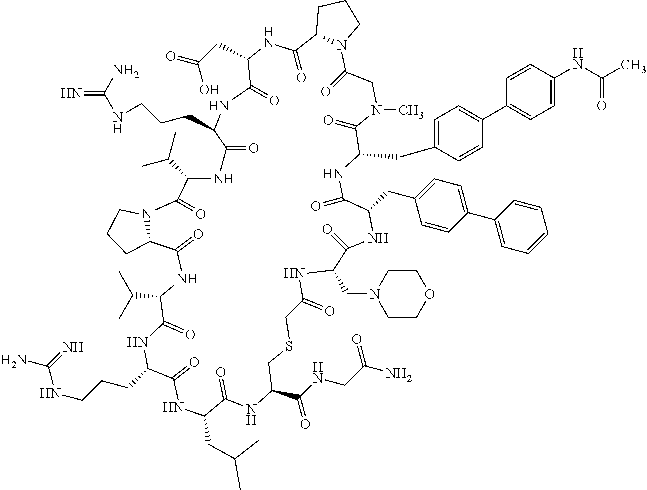 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 128 was prepared on a 50 umol scale. It was purified by Preparative LC/MS. The yield of the product was 26.3 mg, and its estimated purity by LCMS analysis was 94.4%. Analysis condition 1: Retention time=1.37 min. ESI-MS(+) m/z [M+2H] 2+: 941.3.

### Preparation of Example 129

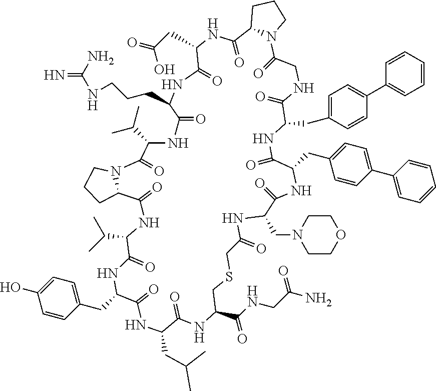 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CNC(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 129 was prepared on a 50 umol scale. It was purified by Preparative LC/MS. The yield of the product was 17.4 mg, and its estimated purity by LCMS analysis was 97.3%. Analysis condition 2: Retention time=1.59 min. ESI-MS(+) m/z [M+2H] 2+: 909.1.

### Preparation of Example 130

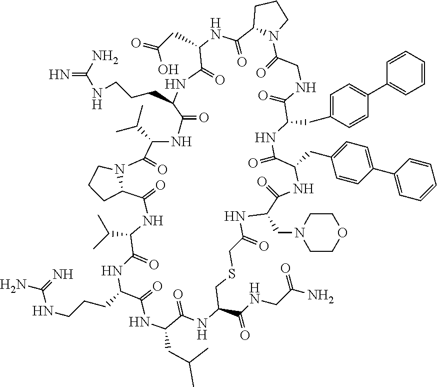 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CNC(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 130 was prepared on a 50 umol scale. It was purified by Preparative LC/MS. The yield of the product was 18 mg, and its estimated purity by LCMS analysis was 100%. Analysis condition 1: Retention time=1.43 min. ESI-MS(+) m/z [M+H]⁺: 1810.

### Preparation of Example 130

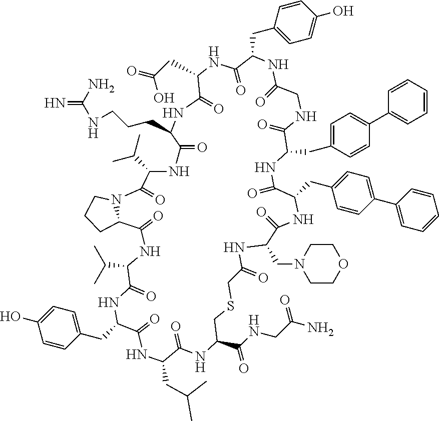 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CNC(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 131 was prepared on a 50 umol scale. It was purified by Preparative LC/MS. The yield of the product was 27.2 mg, and its estimated purity by LCMS analysis was 100%. Analysis condition 1: Retention time=1.57 min. ESI-MS(+) m/z [M+H]⁺: 1884.

### Preparation of Example 132

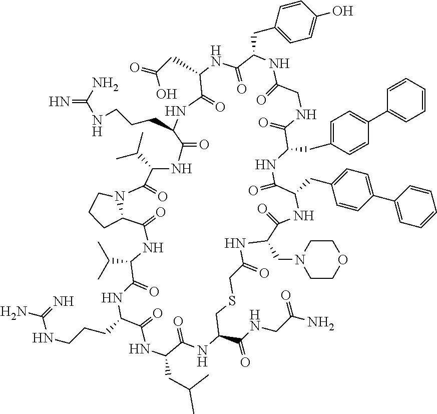 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CNC(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 132 was prepared on a 0.1 umol scale. It was purified by Preparative LC/MS. The yield of the product was 18.3 mg, and its estimated purity by LCMS analysis was 98.9%. Analysis condition 1: Retention time=1.46 min. ESI-MS(+) m/z [M+H]⁺: 1876.

### Preparation of Example 133

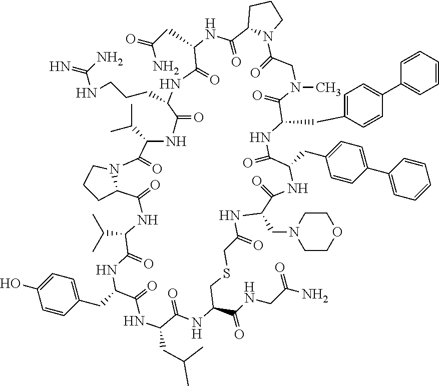 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@H](CCCNC(=N)N)NC(=O)[C@H](CC(N)=O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 133 was prepared on a 50 umol scale. It was purified by Preparative LC/MS. The yield of the product was 26.3 mg, and its estimated purity by LCMS analysis was 98.7%. Analysis condition 2: Retention time=1.73 min. ESI-MS(+) m/z [M+2H] 2+: 916.

### Preparation of Example 134

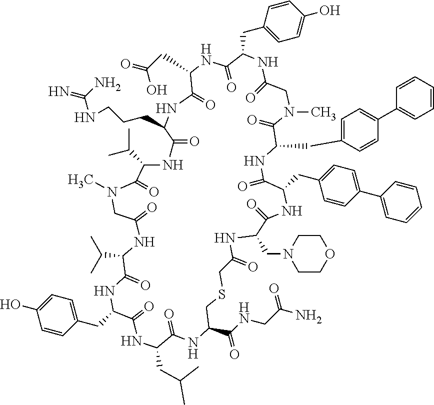 [SMILES: CC(C)C[C@@H]1NC(=O)[C@H](Cc2ccc(O)cc2)NC(=O)[C@H](C(C)C)NC(=O)CN(C)C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 134 was prepared on a 50 umol scale. It was purified by Preparative LC/MS. The yield of the product was 22.9 mg, and its estimated purity by LCMS analysis was 95.7%. Analysis condition 1: Retention time=1.86 min. ESI-MS(+) m/z [M+H]⁺: 1871.1.

### Preparation of Example 135

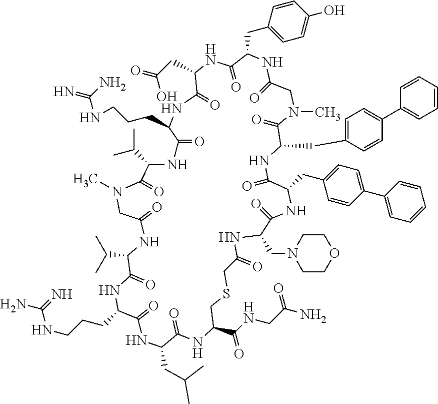 [SMILES: CC(C)C[C@@H]1NC(=O)[C@H](CCCNC(=N)N)NC(=O)[C@H](C(C)C)NC(=O)CN(C)C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 135 was prepared on a 50 umol scale. It was purified by Preparative LC/MS. The yield of the product was 14.7 mg, and its estimated purity by LCMS analysis was 92%. Analysis condition 2: Retention time=1.52 min. ESI-MS(+) m/z [M+H]⁺: 1863.8.

### Preparation of Example 136

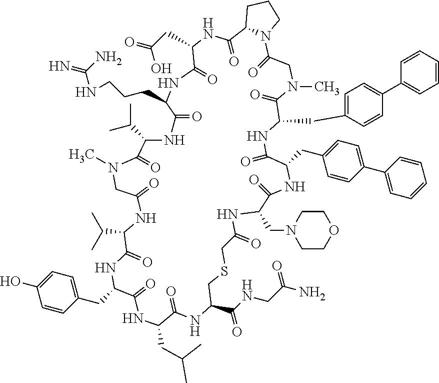 [SMILES: CC(C)C[C@@H]1NC(=O)[C@H](Cc2ccc(O)cc2)NC(=O)[C@H](C(C)C)NC(=O)CN(C)C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 136 was prepared on a 50 umol scale. It was purified by Preparative LC/MS. The yield of the product was 24.9 mg, and its estimated purity by LCMS analysis was 96.8%. Analysis condition 1: Retention time=1.85 min. ESI-MS(+) m/z [M+H]⁺: 1805.1.

### Preparation of Example 137

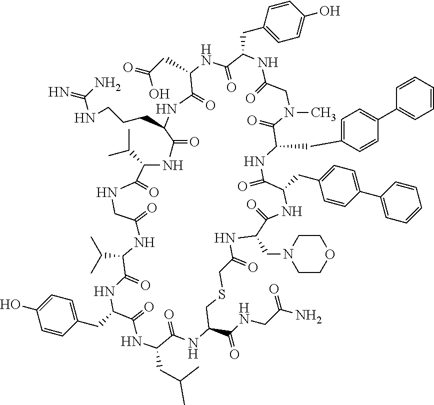 [SMILES: CC(C)C[C@@H]1NC(=O)[C@H](Cc2ccc(O)cc2)NC(=O)[C@H](C(C)C)NC(=O)CNC(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 137 was prepared on a 0.1 umol scale. It was purified by Preparative LC/MS. The yield of the product was 23.3 mg, and its estimated purity by LCMS analysis was 95%. Analysis condition 1: Retention time=1.81 min. ESI-MS(+) m/z [M+H]⁺: 1857.2.

### Preparation of Example 138

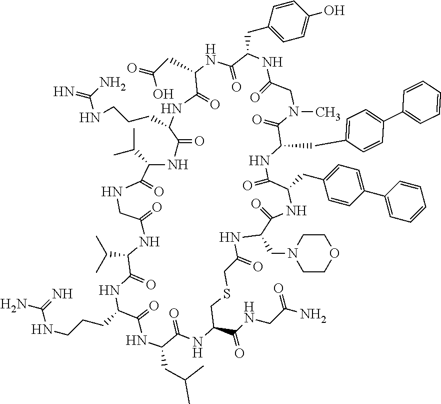 [SMILES: CC(C)C[C@@H]1NC(=O)[C@H](CCCNC(=N)N)NC(=O)[C@H](C(C)C)NC(=O)CNC(=O)[C@H](C(C)C)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 138 was prepared on a 50 umol scale. It was purified by Preparative LC/MS. The yield of the product was 13.4 mg, and its estimated purity by LCMS analysis was 95.2%. Analysis condition 1: Retention time=1.76 min. ESI-MS(+) m/z [M+H]⁺: 1850.1.

### Preparation of Example 139

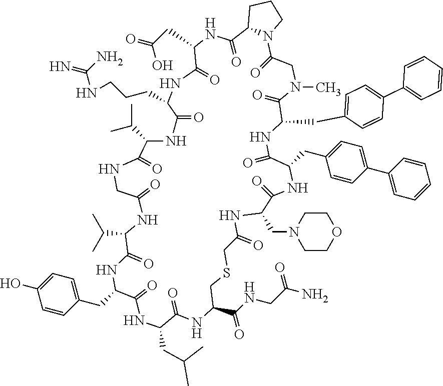 [SMILES: CC(C)C[C@@H]1NC(=O)[C@H](Cc2ccc(O)cc2)NC(=O)[C@H](C(C)C)NC(=O)CNC(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 139 was prepared on a 50 umol scale. It was purified by Preparative LC/MS. The yield of the product was 25 mg, and its estimated purity by LCMS analysis was 95.9%. Analysis condition 2: Retention time=1.66 min. ESI-MS(+) m/z [M+H]⁺: 1791.3.

### Preparation of Example 140

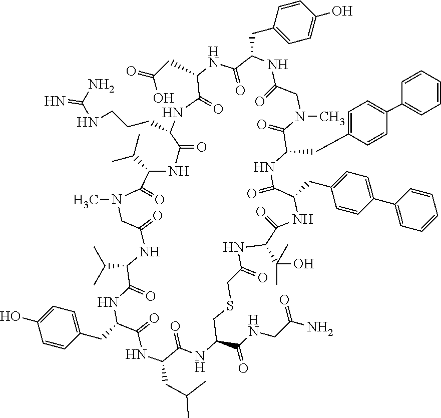 [SMILES: CC(C)C[C@@H]1NC(=O)[C@H](Cc2ccc(O)cc2)NC(=O)[C@H](C(C)C)NC(=O)CN(C)C(=O)[C@H](C(C)C)NC(=O)[C@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](C(C)(C)O)NC(=O)CSC[C@@H](C(=O)NCC(N)=O)NC1=O]

Example 140 was prepared on a 50 umol scale. It was purified by Preparative LC/MS. The yield of the product was 15.6 mg, and its estimated purity by LCMS analysis was 92.3%. Analysis condition 2: Retention time=1.87 min. ESI-MS(+) m/z [M+H]⁺: 1830.1.

### Preparation of Example 141

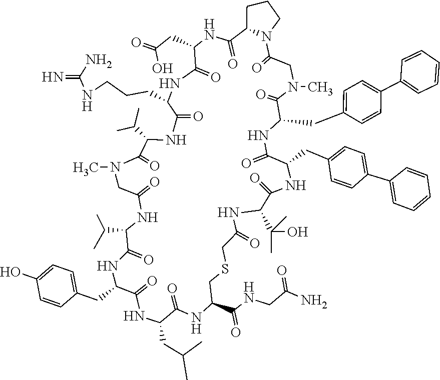 [SMILES: CC(C)C[C@@H]1NC(=O)[C@H](Cc2ccc(O)cc2)NC(=O)[C@H](C(C)C)NC(=O)CN(C)C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](C(C)(C)O)NC(=O)CSC[C@@H](C(=O)NCC(N)=O)NC1=O]

Example 141 was prepared on a 50 umol scale. It was purified by Preparative LC/MS. The yield of the product was 21.3 mg, and its estimated purity by LCMS analysis was 96.6%. Analysis condition 2: Retention time=1.77 min. ESI-MS(+) m/z [M+2H] 2+: 883.2.

### Preparation of Example 142

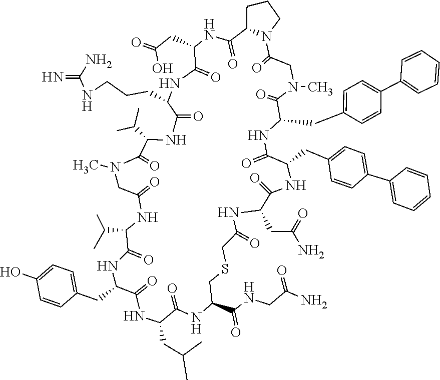 [SMILES: CC(C)C[C@@H]1NC(=O)[C@H](Cc2ccc(O)cc2)NC(=O)[C@H](C(C)C)NC(=O)CN(C)C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CC(N)=O)NC(=O)CSC[C@@H](C(=O)NCC(N)=O)NC1=O]

Example 142 was prepared on a 50 umol scale. It was purified by Preparative LC/MS. The yield of the product was 22.8 mg, and its estimated purity by LCMS analysis was 98.9%. Analysis condition 2: Retention time=1.82 min. ESI-MS(+) m/z [M+H]⁺: 1763.7.

### Preparation of Example 143

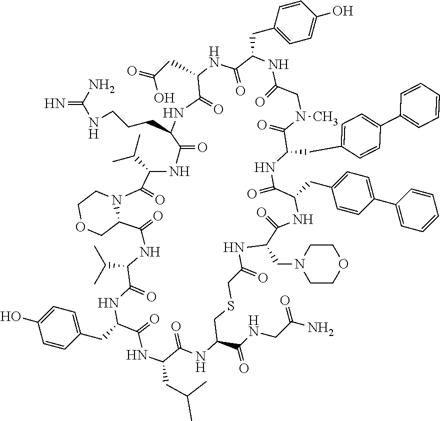 [SMILES: CC(C)C[C@@H]1NC(=O)[C@H](Cc2ccc(O)cc2)NC(=O)[C@H](C(C)C)NC(=O)[C@@H]2COCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 143 was prepared on a 50 umol scale. It was purified by Preparative LC/MS. The yield of the product was 26.6 mg, and its estimated purity by LCMS analysis was 95.3%. Analysis condition 1: Retention time=1.67 min. ESI-MS(+) m/z [M+H]⁺: 1913.1.

### Preparation of Example 144

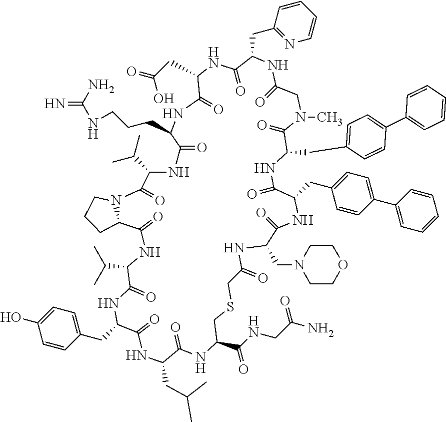 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 144 was prepared on a 50 umol scale. It was purified by Preparative LC/MS. The yield of the product was 13.3 mg, and its estimated purity by LCMS analysis was 98.4%. Analysis condition 1: Retention time=1.64 min. ESI-MS(+) m/z [M+H]⁺: 1882.1.

### Preparation of Example 145

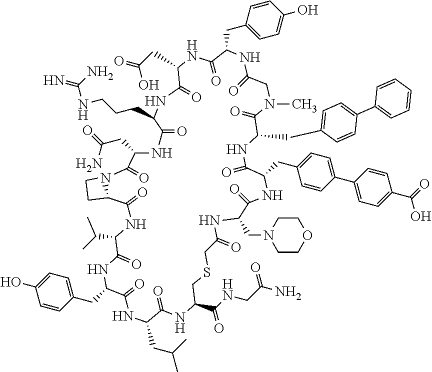 [SMILES: CC(C)C[C@@H]1NC(=O)[C@H](Cc2ccc(O)cc2)NC(=O)[C@H](C(C)C)NC(=O)[C@@H]2CCN2C(=O)[C@H](CC(N)=O)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC1=O]

Example 145 was prepared on a 50 Preparative LC/MS. The yield of the product was 34.3 mg, and its estimated purity by LCMS analysis was 92.2%. Analysis condition 1: Retention time=1.45 min. ESI-MS(+) m/z [M+H]⁺: 1941.9.

### Preparation of Example 146

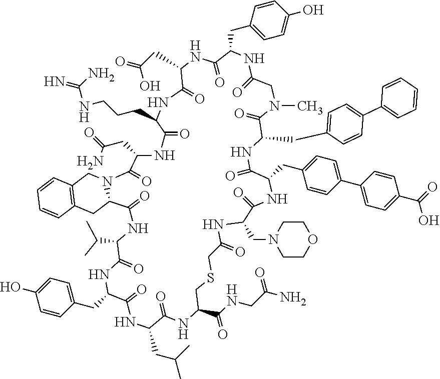 [SMILES: CC(C)C[C@@H]1NC(=O)[C@H](Cc2ccc(O)cc2)NC(=O)[C@H](C(C)C)NC(=O)[C@@H]2Cc3ccccc3CN2C(=O)[C@H](CC(N)=O)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC1=O]

Example 146 was prepared on a 50 umol scale. It was purified by Preparative LC/MS. The yield of the product was 27.6 mg, and its estimated purity by LCMS analysis was 96.9%. Analysis condition 1: Retention time=1.52 min. ESI-MS(+) m/z [M+2H] 2+: 1010.1.

### Preparation of Example 147

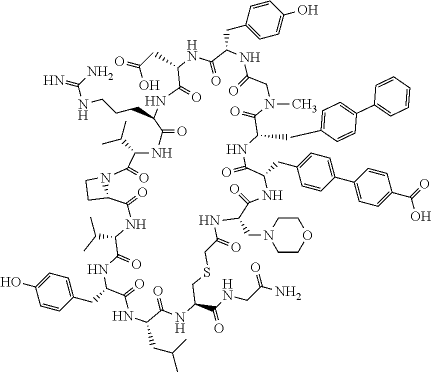 [SMILES: CC(C)C[C@@H]1NC(=O)[C@H](Cc2ccc(O)cc2)NC(=O)[C@H](C(C)C)NC(=O)[C@@H]2CCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC1=O]

Example 147 was prepared on a 50 Preparative LC/MS 28100. The yield of the product was 24.8 mg, and its estimated purity by LCMS analysis was 92.7%. Analysis condition 13: Retention time=1.45 min. ESI-MS(+) m/z [M+H]⁺: 1928.1.

### Preparation of Example 148

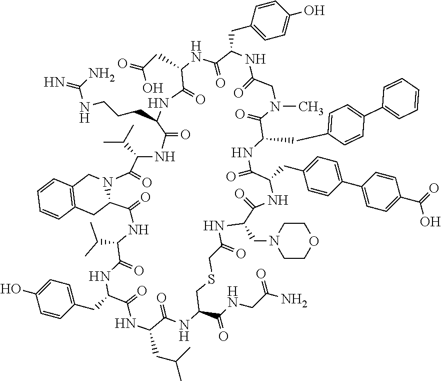 [SMILES: CC(C)C[C@@H]1NC(=O)[C@H](Cc2ccc(O)cc2)NC(=O)[C@H](C(C)C)NC(=O)[C@@H]2Cc3ccccc3CN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC1=O]

Example 148 was prepared on a 50 umol scale. It was purified by Preparative LC/MS. The yield of the product was 52.4 mg, and its estimated purity by LCMS analysis was 97.9%. Analysis condition 1: Retention time=1.54 min. ESI-MS(+) m/z [M+2H] 2+: 1002.2.

### Preparation of Example 149

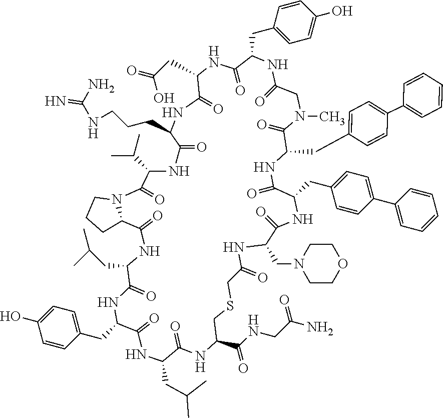 [SMILES: CC(C)C[C@@H]1NC(=O)[C@H](Cc2ccc(O)cc2)NC(=O)[C@H](CC(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 149 was prepared on a 50 umol scale. It was purified by Preparative LC/MS. The yield of the product was 10 mg, and its estimated purity by LCMS analysis was 97.5%. Analysis condition 2: Retention time=1.66 min. ESI-MS(+) m/z [M+2H] 2+: 956.4.

### Preparation of Example 150

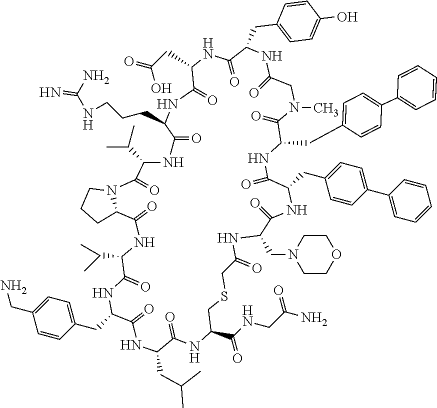 [SMILES: CC(C)C[C@@H]1NC(=O)[C@H](Cc2ccc(CN)cc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 150 was prepared on a 50 umol scale. It was purified by Preparative LC/MS. The yield of the product was 2.6 mg, and its estimated purity by LCMS analysis was 92.6%. Analysis condition 1: Retention time=1.55 min. ESI-MS(+) m/z [M+2H] 2+: 955.4.

### Preparation of Example 151

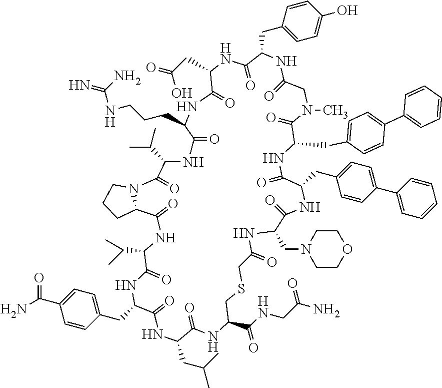 [SMILES: CC(C)C[C@@H]1NC(=O)[C@H](Cc2ccc(C(N)=O)cc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 151 was prepared on a 50 umol scale. It was purified by Preparative LC/MS. The yield of the product was 22 mg, and its estimated purity by LCMS analysis was 100%. Analysis condition 2: Retention time=1.53 min. ESI-MS(+) m/z [M+H]⁺: 1925.1.

### Preparation of Example 152

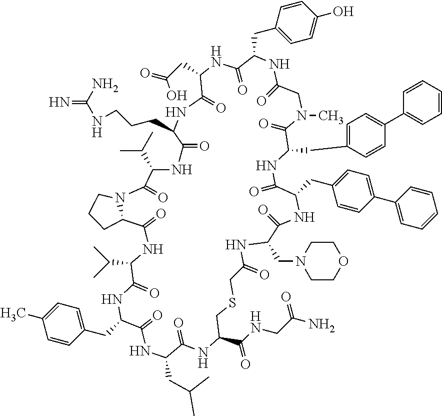 [SMILES: Cc1ccc(C[C@@H]2NC(=O)[C@H](C(C)C)NC(=O)[C@@H]3CCCN3C(=O)[C@H](C(C)C)NC(=O)[C@@H](CCCNC(=N)N)NC(=O)[C@H](CC(=O)O)NC(=O)[C@H](Cc3ccc(O)cc3)NC(=O)CN(C)C(=O)[C@H](Cc3ccc(-c4ccccc4)cc3)NC(=O)[C@H](Cc3ccc(-c4ccccc4)cc3)NC(=O)[C@H](CN3CCOCC3)NC(=O)CSC[C@@H](C(=O)NCC(N)=O)NC(=O)[C@H](CC(C)C)NC2=O)cc1]

Example 152 was prepared on a 50 umol scale. It was purified by Preparative LC/MS. The yield of the product was 29.9 mg, and its estimated purity by LCMS analysis was 97.1%. Analysis condition 2: Retention time=1.68 min. ESI-MS(+) m/z [M+H]⁺: 1895.1.

### Preparation of Example 153

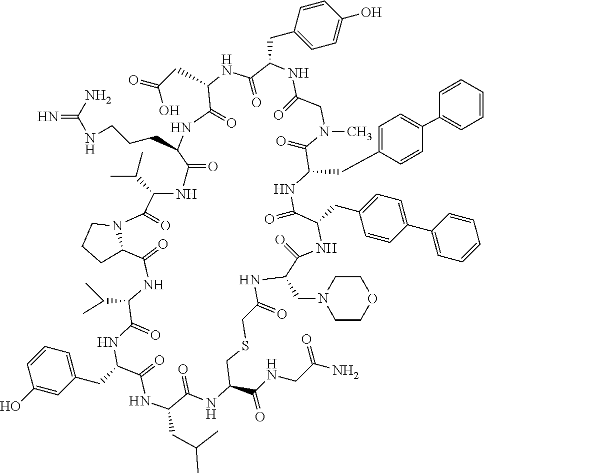 [SMILES: CC(C)C[C@@H]1NC(=O)[C@H](Cc2cccc(O)c2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 153 was prepared on a 50 umol scale. It was purified by Preparative LC/MS. The yield of the product was 32.9 mg, and its estimated purity by LCMS analysis was 99%. Analysis condition 1: Retention time=1.82 min. ESI-MS(+) m/z [M+2H] 2+: 948.1.

### Preparation of Example 154

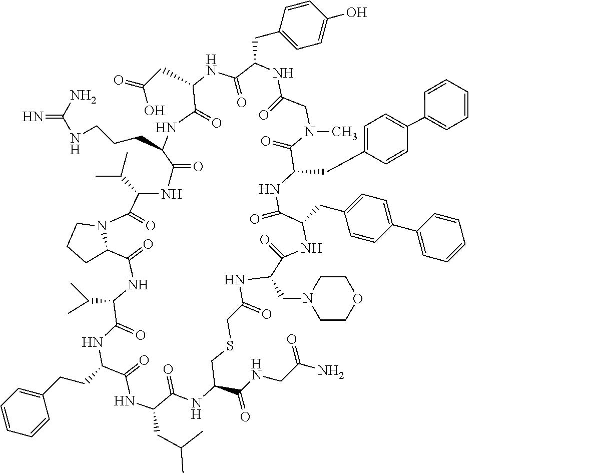 [SMILES: CC(C)C[C@@H]1NC(=O)[C@H](CCc2ccccc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 154 was prepared on a 50 umol scale. It was purified by Preparative LC/MS. The yield of the product was 22.7 mg, and its estimated purity by LCMS analysis was 94.2%. Analysis condition 1: Retention time=1.88 min. ESI-MS(+) m/z [M+2H] 2+: 948.2.

### Preparation of Example 155

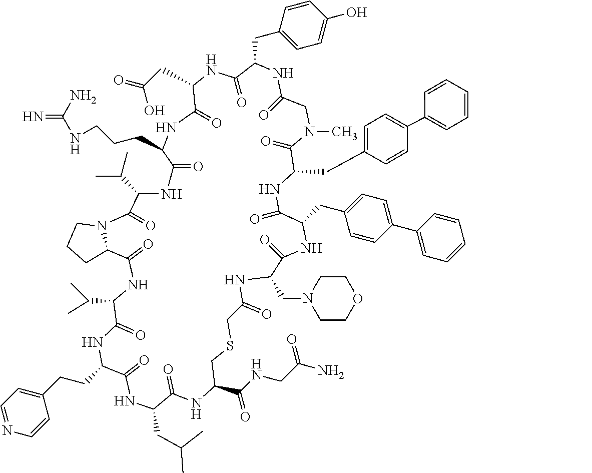 [SMILES: CC(C)C[C@@H]1NC(=O)[C@H](CCc2ccncc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 155 was prepared on a 50 umol scale. It was purified by Preparative LC/MS. The yield of the product was 11 mg, and its estimated purity by LCMS analysis was 100%. Analysis condition 1: Retention time=1.68 min. ESI-MS(+) m/z [M+2H] 2+: 948.9.

### Preparation of Example 156

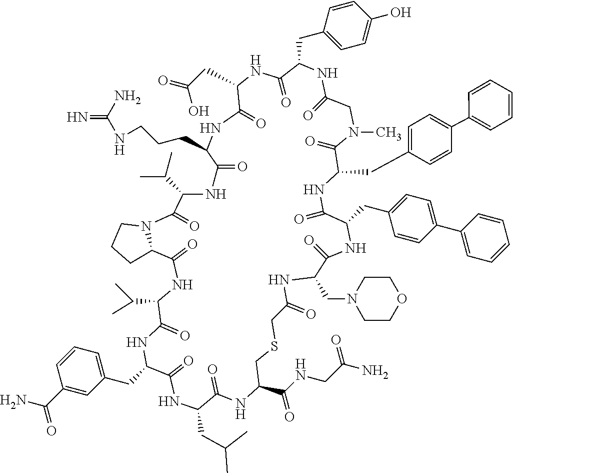 [SMILES: CC(C)C[C@@H]1NC(=O)[C@H](Cc2cccc(C(N)=O)c2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 156 was prepared on a 50 umol scale. It was purified by Preparative LC/MS 107. The yield of the product was 26.2 mg, and its estimated purity by LCMS analysis was 100%. Analysis condition 4: Retention time=1.65 min. ESI-MS(+) m/z [M+2H] 2+: 963.

### Preparation of Example 157

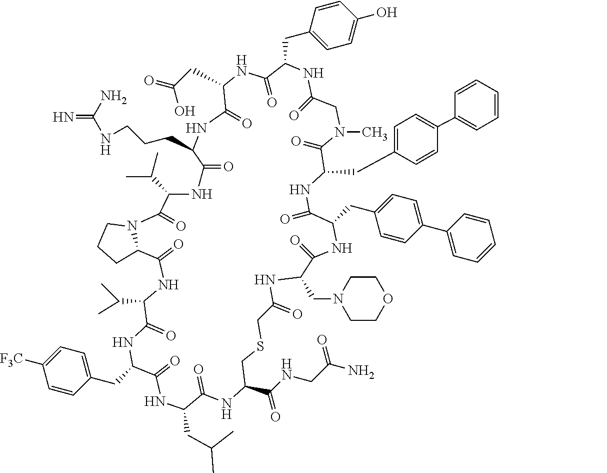 [SMILES: CC(C)C[C@@H]1NC(=O)[C@H](Cc2ccc(C(F)(F)F)cc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 157 was prepared on a 50 umol scale. It was purified by Preparative LC/MS. The yield of the product was 30.1 mg, and its estimated purity by LCMS analysis was 95.8%. Analysis condition 2: Retention time=1.75 min. ESI-MS(+) m/z [M+H]⁺: 1949.2.

### Preparation of Example 158

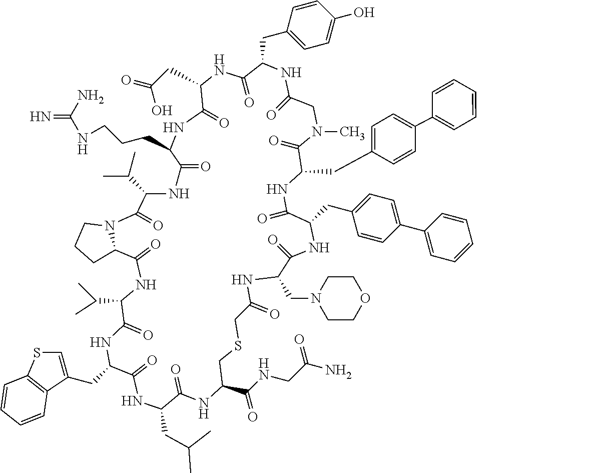 [SMILES: CC(C)C[C@@H]1NC(=O)[C@H](Cc2csc3ccccc23)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 158 was prepared on a 50 umol scale. It was purified by Preparative LC/MS. The yield of the product was 38.5 mg, and its estimated purity by LCMS analysis was 95.8%. Analysis condition 1: Retention time=1.79 min. ESI-MS(+) m/z [M+H]⁺: 1937.1.

### Preparation of Example 159

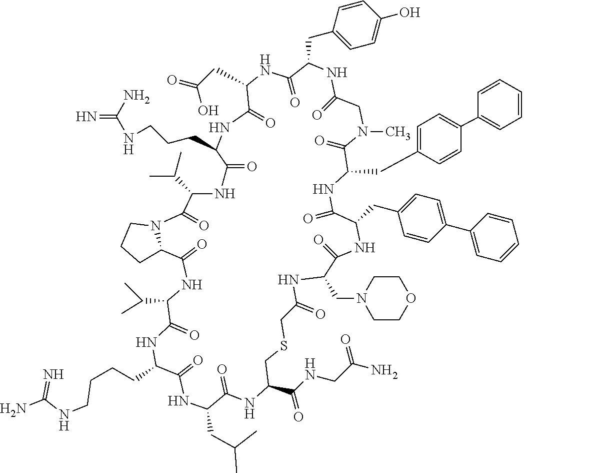 [SMILES: CC(C)C[C@@H]1NC(=O)[C@H](CCCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 159 was prepared on a 50 umol scale. It was purified by Preparative LC/MS. The yield of the product was 26.5 mg, and its estimated purity by LCMS analysis was 99.1%. Analysis condition 1: Retention time=1.64 min. ESI-MS(+) m/z [M+2H] 2+: 953.1.

### Preparation of Example 160

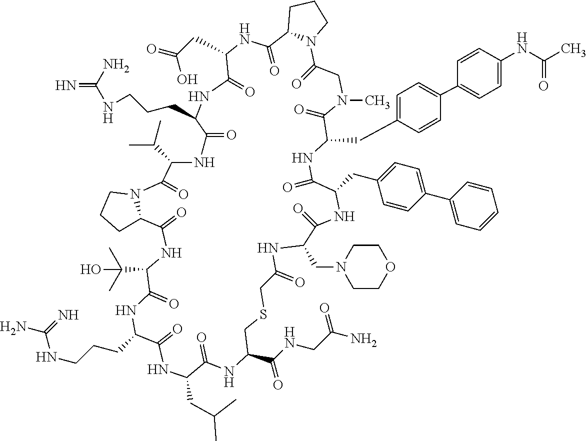 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](CCCNC(=N)N)NC(=O)[C@H](C(C)(C)O)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 160 was prepared on a 50 umol scale. It was purified by Preparative LC/MS. The yield of the product was 16.7 mg, and its estimated purity by LCMS analysis was 98.2%. Analysis condition 1: Retention time=1.4 min. ESI-MS(+) m/z [M+H]⁺: 1897.

### Preparation of Example 161

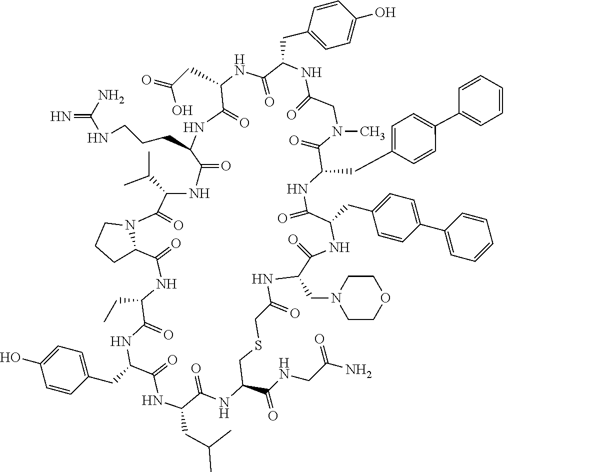 [SMILES: CC[C@@H]1NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2ccc(O)cc2)NC1=O]

Example 161 was prepared on a 50 umol scale. It was purified by Preparative LC/MS. The yield of the product was 27 mg, and its estimated purity by LCMS analysis was 93.7%. Analysis condition 1: Retention time=1.64 min. ESI-MS(+) m/z [M+H]⁺: 1883.2.

### Preparation of Example 162

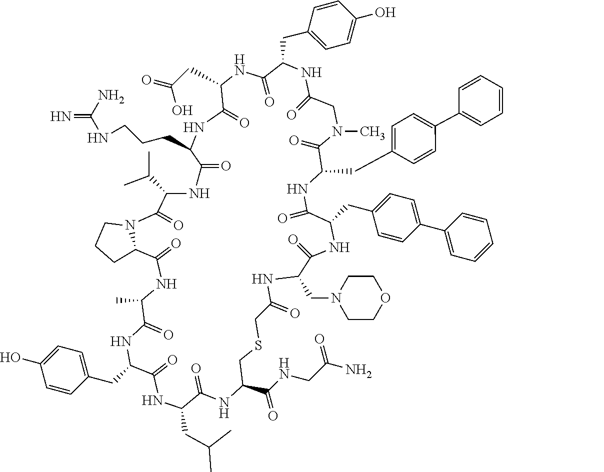 [SMILES: CC(C)C[C@@H]1NC(=O)[C@H](Cc2ccc(O)cc2)NC(=O)[C@H](C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 162 was prepared on a 50 umol scale. It was purified by Preparative LC/MS. The yield of the product was 22.7 mg, and its estimated purity by LCMS analysis was 100%. Analysis condition 1: Retention time=1.76 min. ESI-MS(+) m/z [M+2H] 2+: 935.1.

### Preparation of Example 163

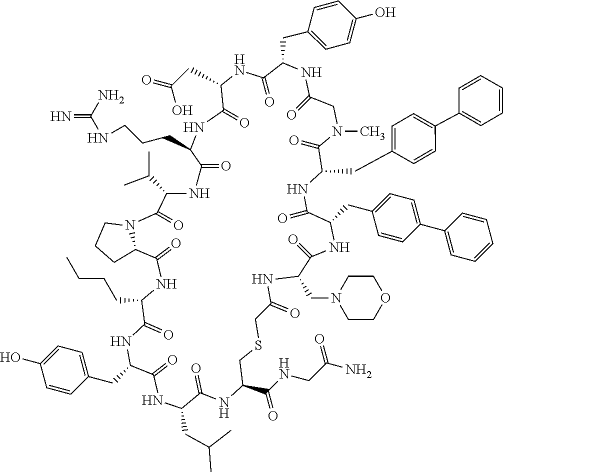 [SMILES: CCCC[C@@H]1NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2ccc(O)cc2)NC1=O]

Example 163 was prepared on a 50 umol scale. It was purified by Preparative LC/MS. The yield of the product was 43.8 mg, and its estimated purity by LCMS analysis was 97.7%. Analysis condition 2: Retention time=1.63 min. ESI-MS(+) m/z [M+H]⁺: 1912.

### Preparation of Example 164

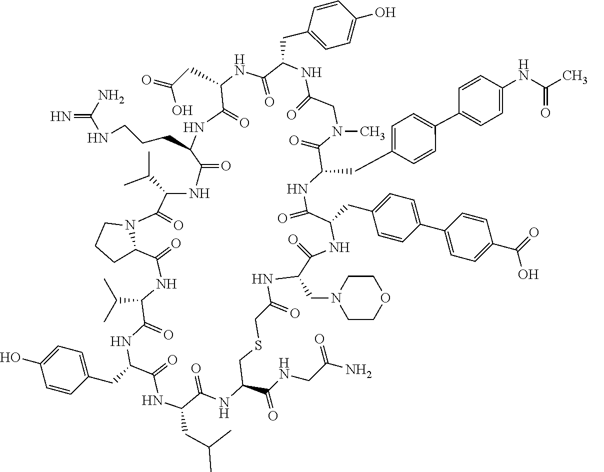 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccc(O)cc4)NC(=O)CN(C)C3=O)cc2)cc1]

Example 164 was prepared on a 50 umol scale. It was purified by Preparative LC/MS. The yield of the product was 14.1 mg, and its estimated purity by LCMS analysis was 100%. Analysis condition 2: Retention time=1.31 min. ESI-MS(+) m/z [M+2H] 2+: 1000.

### Preparation of Example 165

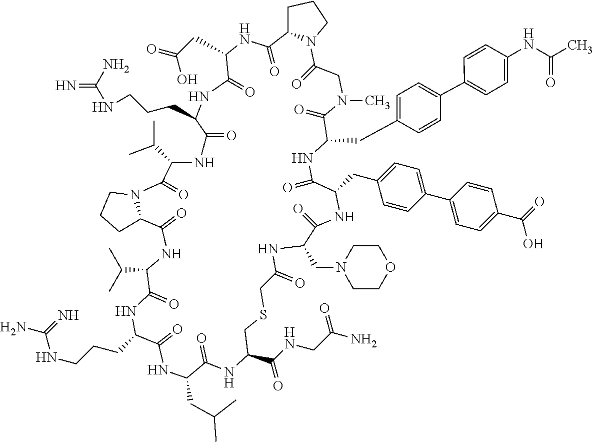 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 165 was prepared on a 50 umol scale. It was purified by Preparative LC/MS. The yield of the product was 8.6 mg, and its estimated purity by LCMS analysis was 91.7%. Analysis condition 2: Retention time=1.22 min. ESI-MS(+) m/z [M+2H] 2+: 963.1.

### Preparation of Example 166

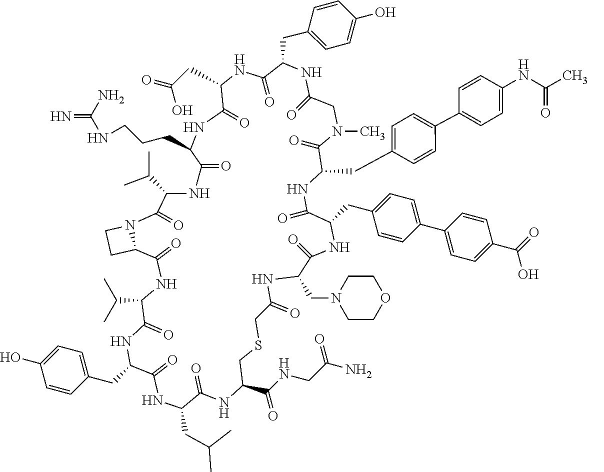 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCN4C(=O)[C@H](C(C)C)NC(=O)[C@@H](CCCNC(=N)N)NC(=O)[C@H](CC(=O)O)NC(=O)[C@H](Cc4ccc(O)cc4)NC(=O)CN(C)C3=O)cc2)cc1]

Example 166 was prepared on a 50 umol scale. It was purified by Preparative LC/MS. The yield of the product was 22.3 mg, and its estimated purity by LCMS analysis was 88.3%. Analysis condition 1: Retention time=1.27 min. ESI-MS(+) m/z [M+2H] 2+: 992.2.

### Preparation of Example 167

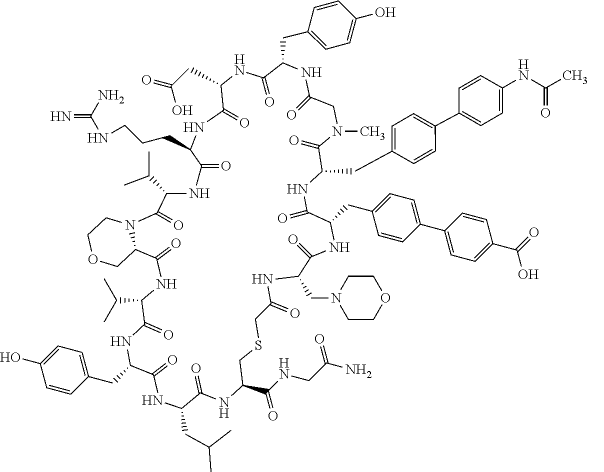 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4COCCN4C(=O)[C@H](C(C)C)NC(=O)[C@@H](CCCNC(=N)N)NC(=O)[C@H](CC(=O)O)NC(=O)[C@H](Cc4ccc(O)cc4)NC(=O)CN(C)C3=O)cc2)cc1]

Example 167 was prepared on a 40 umol scale. It was purified by Preparative LC/MS. The yield of the product was 31.5 mg, and its estimated purity by LCMS analysis was 95.8%. Analysis condition 1: Retention time=1.36 min. ESI-MS(+) m/z [M+2H] 2+: 1008.1.

### Preparation of Example 168

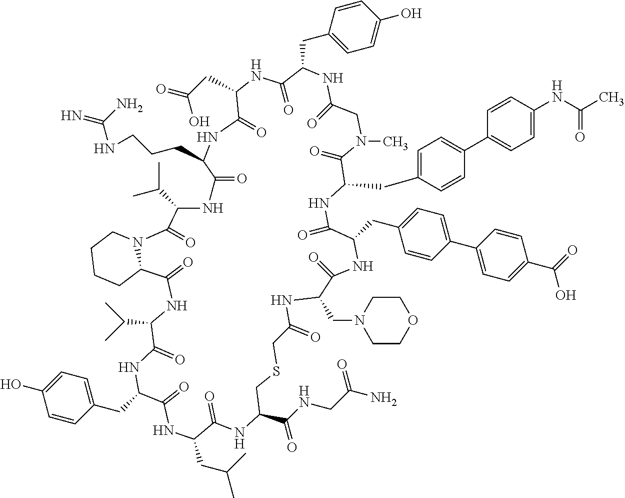 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCCN4C(=O)[C@H](C(C)C)NC(=O)[C@@H](CCCNC(=N)N)NC(=O)[C@H](CC(=O)O)NC(=O)[C@H](Cc4ccc(O)cc4)NC(=O)CN(C)C3=O)cc2)cc1]

Example 168 was prepared on a 50 umol scale. It was purified by Preparative LC/MS. The yield of the product was 24.2 mg, and its estimated purity by LCMS analysis was 92.1%. Analysis condition 1: Retention time=1.35 min. ESI-MS(+) m/z [M+2H] 2+: 1007.1.

### Preparation of Example 169

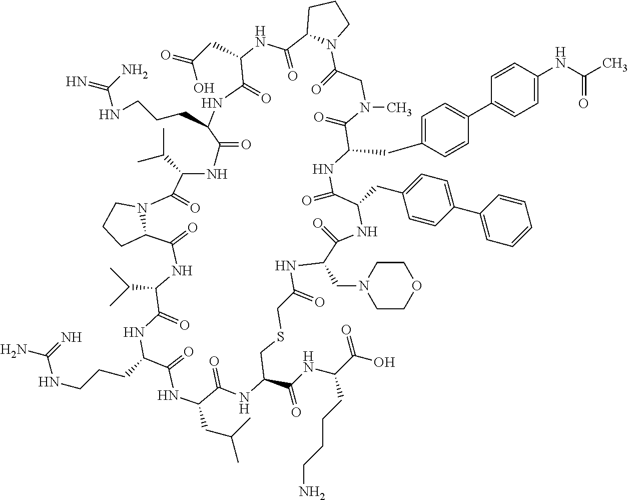 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)N[C@@H](CCCCN)C(=O)O)NC(=O)[C@H](CC(C)C)NC(=O)[C@H](CCCNC(=N)N)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 169 was prepared on a 50 umol scale. It was purified by Preparative LC/MS. The yield of the product was 25.6 mg, and its estimated purity by LCMS analysis was 91.3%. Analysis condition 1: Retention time=1.29 min. ESI-MS(+) m/z [M+2H] 2+: 977.1.

### Preparation of Example 170

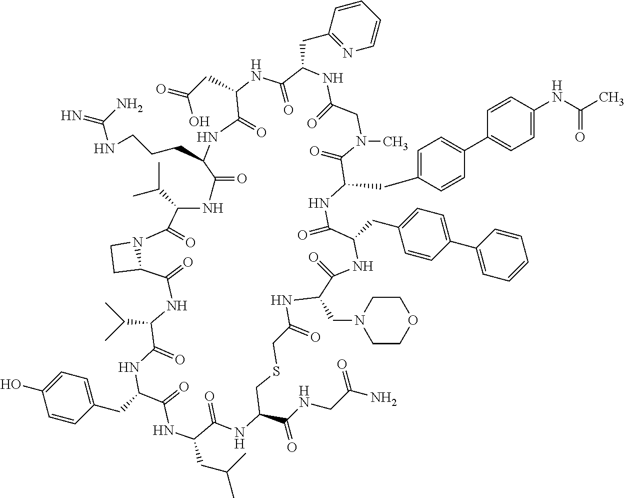 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 170 was prepared on a 50 Preparative LC/MS. The yield of the product was 16.1 mg, and its estimated purity by LCMS analysis was 95.5%. Analysis condition 2: Retention time=1.39 min. ESI-MS(+) m/z [M+H]⁺: 1926.3.

### Preparation of Example 171

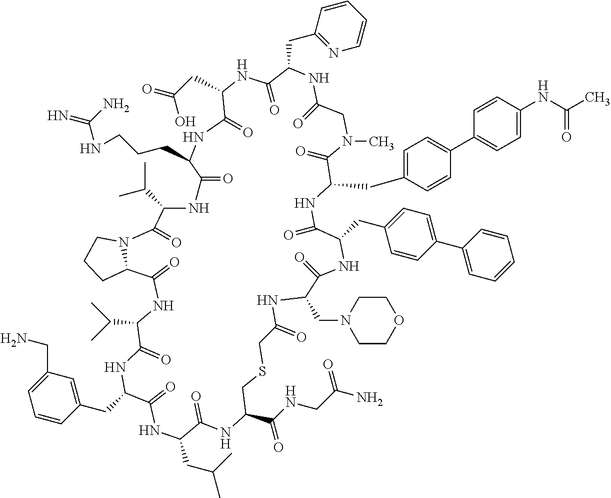 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cccc(CN)c4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 171 was prepared on a 50 umol scale. It was purified by Preparative LC/MS. The yield of the product was 33 mg, and its estimated purity by LCMS analysis was 97.6%. Analysis condition 1: Retention time=1.51 min. ESI-MS(+) m/z [M+2H] 2+: 977.1.

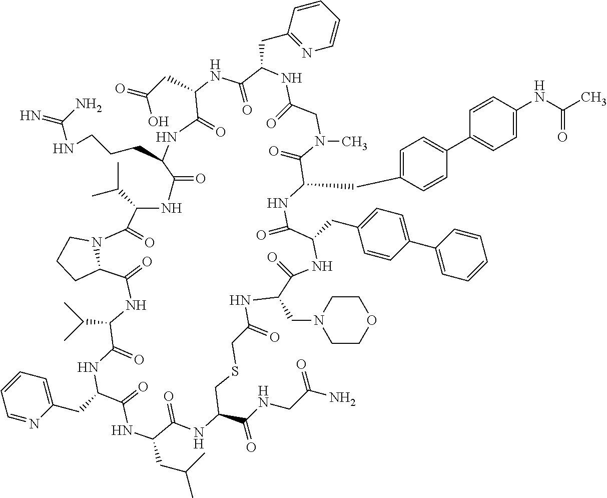 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccccn4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 172 was prepared on a 50 Preparative LC/MS. The yield of the product was 21 mg, and its estimated purity by LCMS analysis was 100%. Analysis condition 1: Retention time=1.61 min. ESI-MS(+) m/z [M+H]⁺: 1924.1.

### Preparation of Example 173

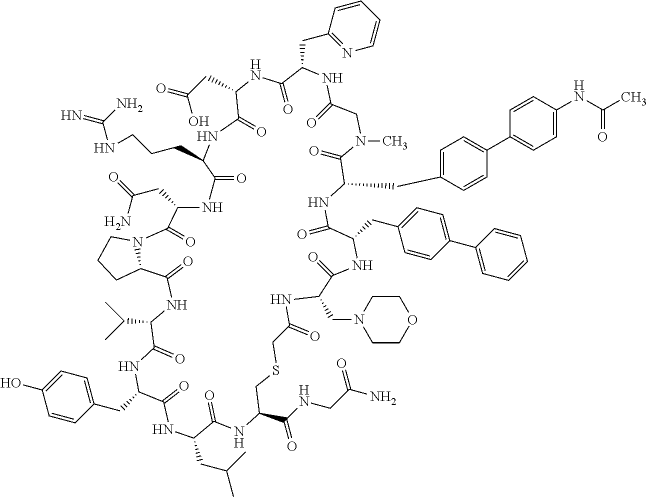 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](CC(N)=O)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 173 was prepared on a 50 umol scale. It was purified by Preparative LC/MS. The yield of the product was 40.2 mg, and its estimated purity by LCMS analysis was 99.2%. Analysis condition 1: Retention time=1.46 min. ESI-MS(+) m/z [M+H]⁺: 1954.

### Preparation of Example 174

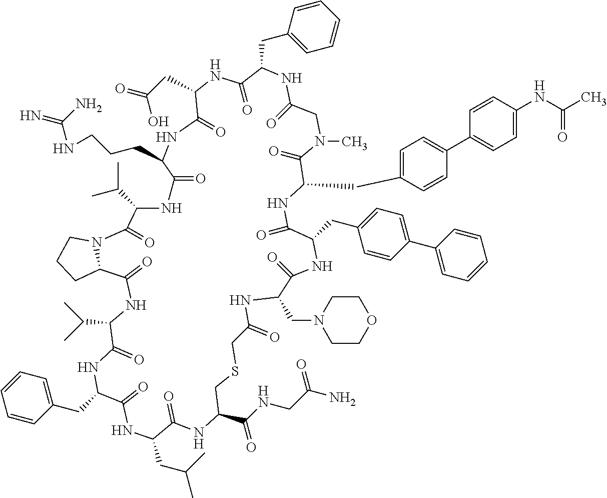 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccccc4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccc4)NC(=O)CN(C)C3=O)cc2)cc1]

Example 174 was prepared on a 50 Preparative LC/MS. The yield of the product was 27.1 mg, and its estimated purity by LCMS analysis was 95%. Analysis condition 1: Retention time=1.43 min. ESI-MS(+) m/z [M+2H] 2+: 962.1.

### Preparation of Example 175

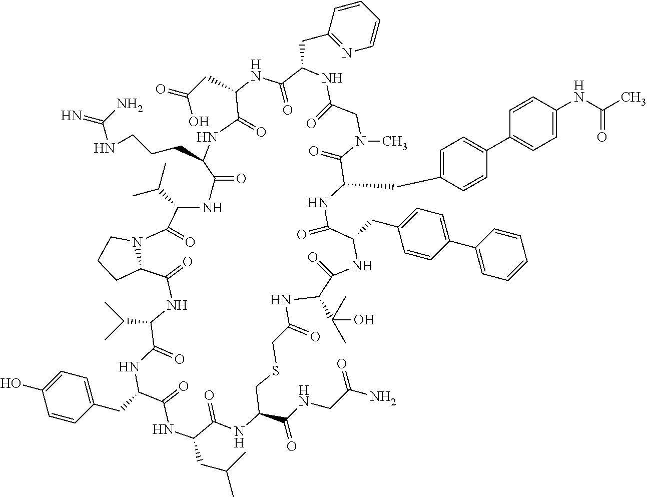 [SMILES: CC(=O)Nc1ccc(-c2ccc(C[C@@H]3NC(=O)[C@H](Cc4ccc(-c5ccccc5)cc4)NC(=O)[C@H](C(C)(C)O)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 175 was prepared on a 50 umol scale. It was purified by Preparative LC/MS. The yield of the product was 29.6 mg, and its estimated purity by LCMS analysis was 94.6%. Analysis condition 1: Retention time=1.49 min. ESI-MS(+) m/z [M+H]⁺: 1899.1.

### Preparation of Example 176

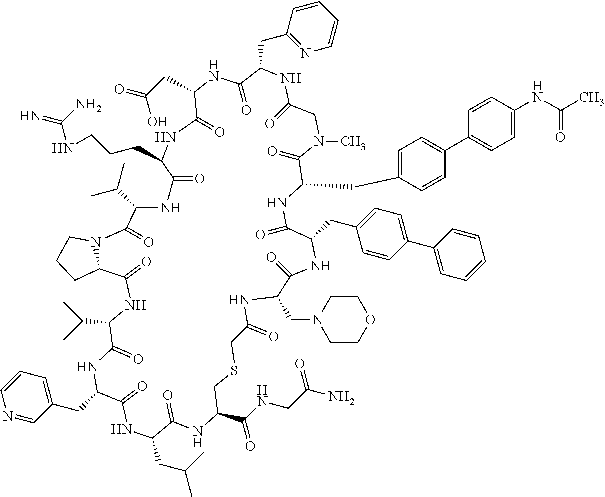 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cccnc4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 176 was prepared on a 50 Preparative LC/MS. The yield of the product was 18.3 mg, and its estimated purity by LCMS analysis was 100%. Analysis condition 2: Retention time=1.25 min. ESI-MS(+) m/z [M+H]⁺: 1923.9.

### Preparation of Example 177

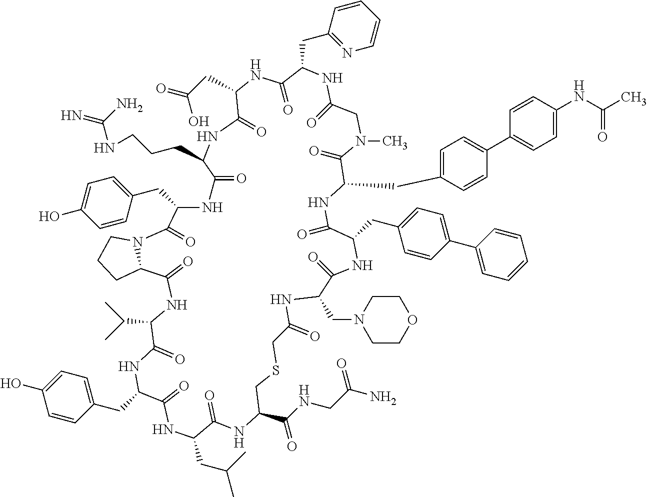 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](Cc4ccc(O)cc4)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 177 was prepared on a 50 umol scale. It was purified by Preparative LC/MS. The yield of the product was 27.5 mg, and its estimated purity by LCMS analysis was 100%. Analysis condition 2: Retention time=1.28 min. ESI-MS(+) m/z [M+2H] 2+: 1002.1.

### Preparation of Example 178

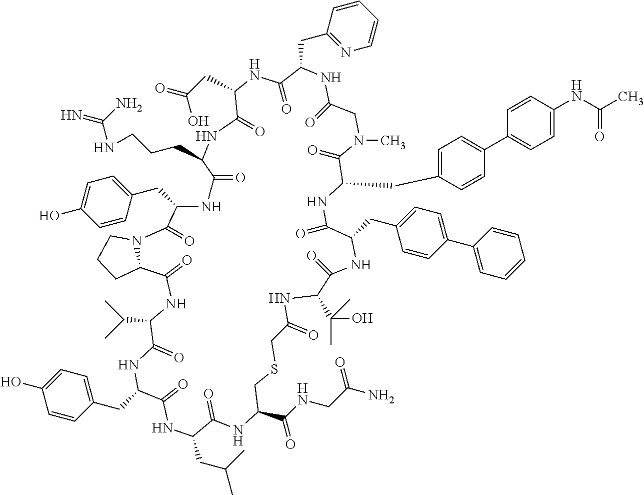 [SMILES: CC(=O)Nc1ccc(-c2ccc(C[C@@H]3NC(=O)[C@H](Cc4ccc(-c5ccccc5)cc4)NC(=O)[C@H](C(C)(C)O)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](Cc4ccc(O)cc4)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 178 was prepared on a 50 Preparative LC/MS. The yield of the product was 34.3 mg, and its estimated purity by LCMS analysis was 95.4%. Analysis condition 1: Retention time=1.56 min. ESI-MS(+) m/z [M+2H] 2+: 982.1.

### Preparation of Example 179

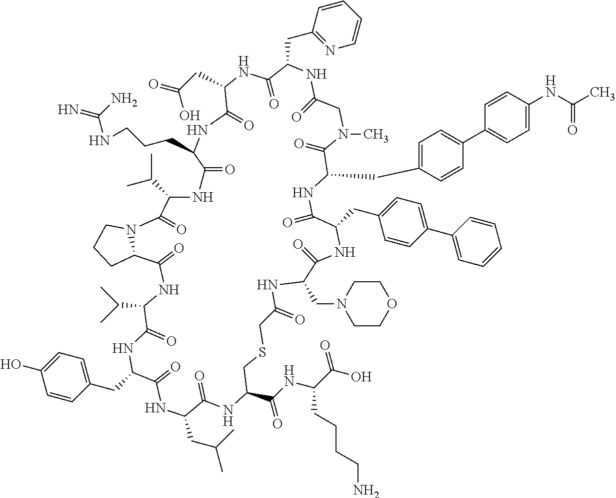 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)N[C@@H](CCCCN)C(=O)O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 179 was prepared on a 50 umol scale. It was purified by Preparative LC/MS. The yield of the product was 34.3 mg, and its estimated purity by LCMS analysis was 97.9%. Analysis condition 1: Retention time=1.45 min. ESI-MS(+) m/z [M+2H] 2+: 1006.2.

### Preparation of Example 180

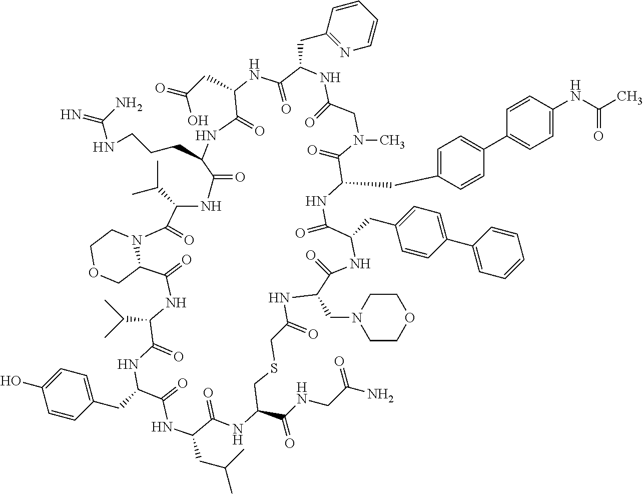 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4COCCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 180 was prepared on a 50 Preparative LC/MS. The yield of the product was 34 mg, and its estimated purity by LCMS analysis was 100%. Analysis condition 2: Retention time=1.3 min. ESI-MS(+) m/z [M+2H] 2+: 978.1.

### Preparation of Example 181

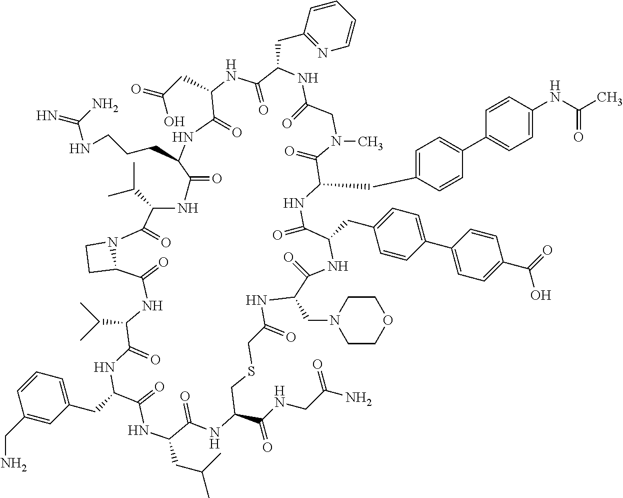 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cccc(CN)c4)NC(=O)[C@H](C(C)C)NC(=O)[C@@H]4CCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 181 was prepared on a 50 umol scale. It was purified by Preparative LC/MS. The yield of the product was 22.8 mg, and its estimated purity by LCMS analysis was 99.2%. Analysis condition 1: Retention time=1.25 min. ESI-MS(+) m/z [M+2H] 2+: 992.1.

### Preparation of Example 182

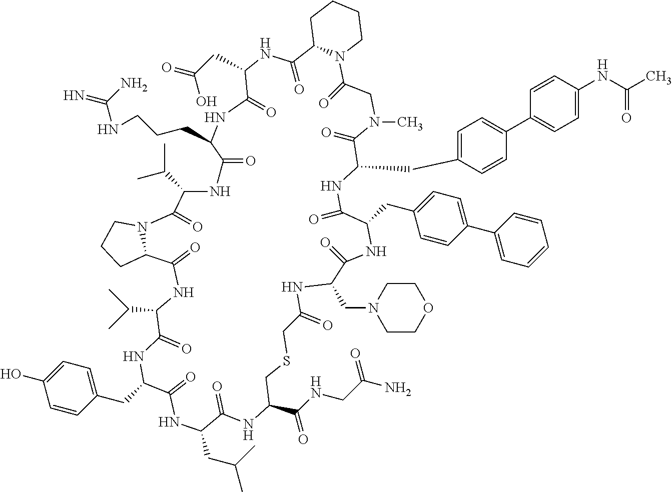 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCCN4C(=O)CN(C)C3=O)cc2)cc1]

Example 182 was prepared on a 50 Preparative LC/MS. The yield of the product was 25.6 mg, and its estimated purity by LCMS analysis was 95.3%. Analysis condition 2: Retention time=1.49 min. ESI-MS(+) m/z [M+H]⁺: 1901.9.

### Preparation of Example 183

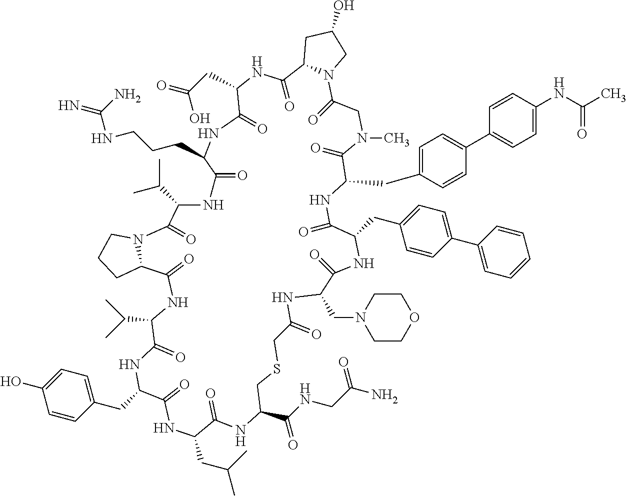 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4C[C@H](O)CN4C(=O)CN(C)C3=O)cc2)cc1]

Example 183 was prepared on a 50 umol scale. It was purified by Preparative LC/MS. The yield of the product was 47 mg, and its estimated purity by LCMS analysis was 96.9%. Analysis condition 2: Retention time=1.39 min. ESI-MS(+) m/z [M+H]⁺: 1903.9.

### Preparation of Example 184

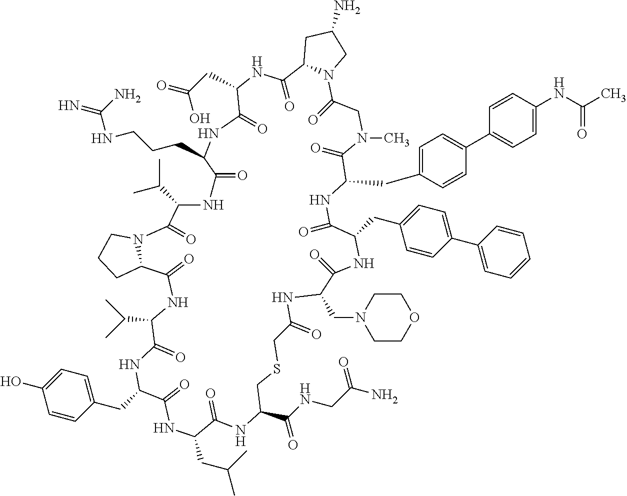 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4C[C@H](N)CN4C(=O)CN(C)C3=O)cc2)cc1]

Example 184 was prepared on a 50 Preparative LC/MS. The yield of the product was 40.5 mg, and its estimated purity by LCMS analysis was 98%. Analysis condition 1: Retention time=1.34 min. ESI-MS(+) m/z [M+H]⁺: 1903.

### Preparation of Example 185

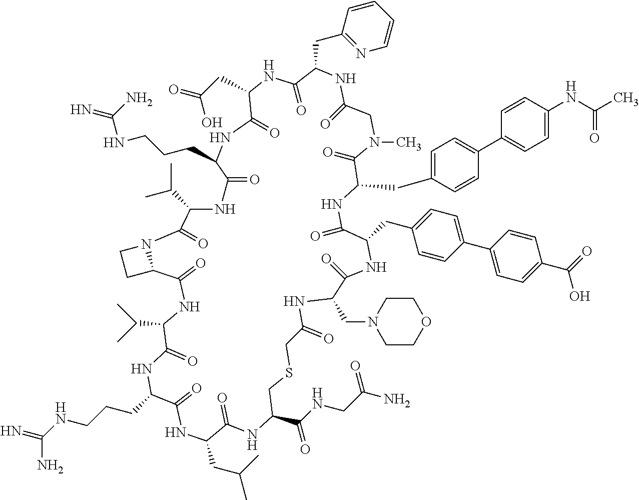 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]4CCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 185 was prepared on a 50 umol scale. It was purified by Preparative LC/MS. The yield of the product was 16.4 mg, and its estimated purity by LCMS analysis was 95.1%. Analysis condition 1: Retention time=1.23 min. ESI-MS(+) m/z [M+H]⁺: 1962.1.

### Preparation of Example 186

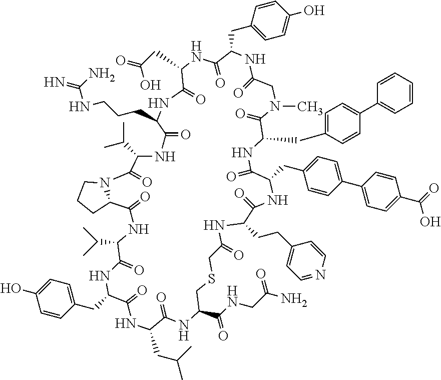 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccc(C(=O)O)cc3)cc2)NC(=O)[C@H](CCc2ccncc2)NC(=O)CSC[C@@H](C(=O)NCC(N)=O)NC1=O]

Example 186 was prepared on a 50 umol scale. It was purified by Preparative LC/MS. The yield of the product was 38.5 mg, and its estimated purity by LCMS analysis was 93.8%. Analysis condition 1: Retention time=1.34 min. ESI-MS(+) m/z [M+2H] 2+: 974.2.

### Preparation of Example 187

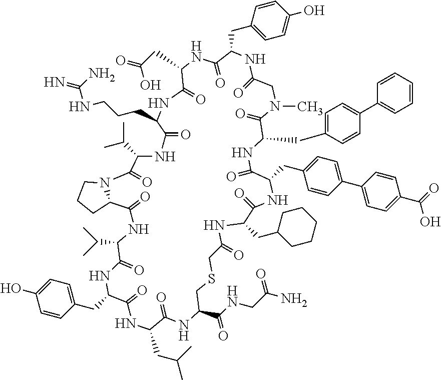 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 187 was prepared on a 50 umol scale. It was purified by Preparative LC/MS. The yield of the product was 33.7 mg, and its estimated purity by LCMS analysis was 100%. Analysis condition 2: Retention time=1.71 min. ESI-MS(+) m/z [M+H]⁺: 1938.

### Preparation of Example 188

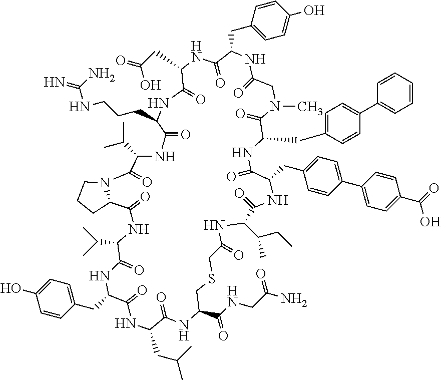 [SMILES: CC[C@H](C)[C@@H]1NC(=O)CSC[C@@H](C(=O)NCC(N)=O)NC(=O)[C@H](CC(C)C)NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccc(C(=O)O)cc3)cc2)NC1=O]

Example 188 was prepared on a 50 umol scale. It was purified by Preparative LC/MS. The yield of the product was 23.7 mg, and its estimated purity by LCMS analysis was 87%. Analysis condition 1: Retention time=1.43 min. ESI-MS(+) m/z [M+2H] 2+: 950.

### Preparation of Example 189

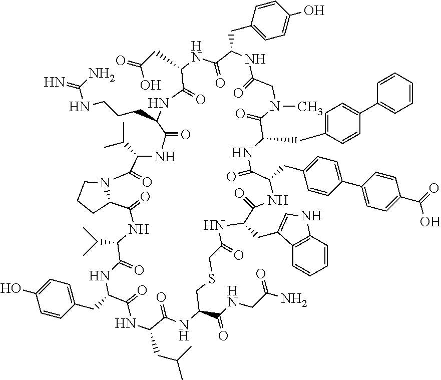 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccc(C(=O)O)cc3)cc2)NC(=O)[C@H](Cc2c[nH]c3ccccc23)NC(=O)CSC[C@@H](C(=O)NCC(N)=O)NC1=O]

Example 189 was prepared on a 50 umol scale. It was purified by Preparative LC/MS. The yield of the product was 34.1 mg, and its estimated purity by LCMS analysis was 100%. Analysis condition 2: Retention time=1.51 min. ESI-MS(+) m/z [M+H]⁺: 1971.1.

### Preparation of Example 190

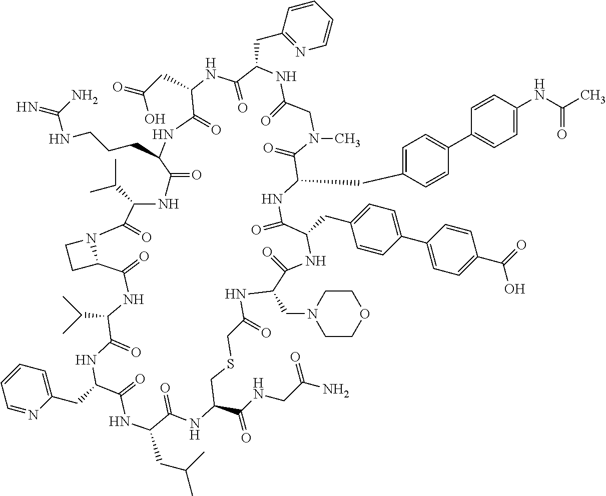 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccccn4)NC(=O)[C@H](C(C)C)NC(=O)[C@@H]4CCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 190 was prepared on a 50 Preparative LC/MS. The yield of the product was 21 mg, and its estimated purity by LCMS analysis was 94.6%. Analysis condition 1: Retention time=1.27 min. ESI-MS(+) m/z [M+H]⁺: 1955.2.

### Preparation of Example 191

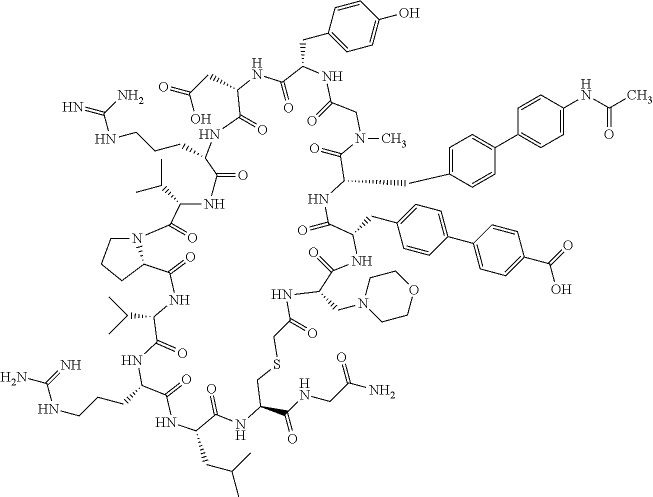 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]4CCCN4C(=O)[C@H](C(C)C)NC(=O)[C@H](CCCNC(=N)N)NC(=O)[C@H](CC(=O)O)NC(=O)[C@H](Cc4ccc(O)cc4)NC(=O)CN(C)C3=O)cc2)cc1]

Example 191 was prepared on a 50 umol scale. It was purified by Preparative LC/MS. The yield of the product was 19.5 mg, and its estimated purity by LCMS analysis was 90.8%. Analysis condition 1: Retention time=1.16 min. ESI-MS(+) m/z [M+2H] 2+: 996.2.

### Preparation of Example 192

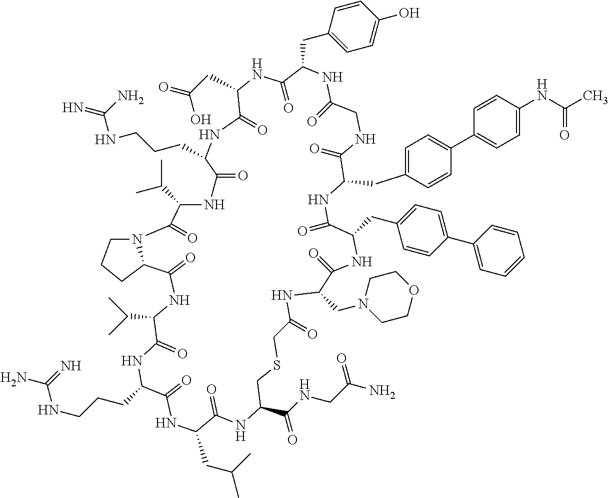 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]4CCCN4C(=O)[C@H](C(C)C)NC(=O)[C@H](CCCNC(=N)N)NC(=O)[C@H](CC(=O)O)NC(=O)[C@H](Cc4ccc(O)cc4)NC(=O)CNC3=O)cc2)cc1]

Example 192 was prepared on a 50 Preparative LC/MS. The yield of the product was 18.1 mg, and its estimated purity by LCMS analysis was 92.8%. Analysis condition 1: Retention time=1.49 min. ESI-MS(+) m/z [M+H]⁺: 1934.

### Preparation of Example 193

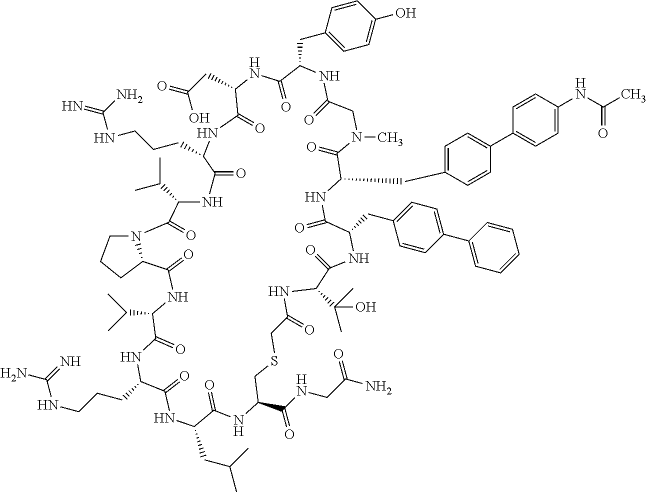 [SMILES: CC(=O)Nc1ccc(-c2ccc(C[C@@H]3NC(=O)[C@H](Cc4ccc(-c5ccccc5)cc4)NC(=O)[C@H](C(C)(C)O)NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]4CCCN4C(=O)[C@H](C(C)C)NC(=O)[C@H](CCCNC(=N)N)NC(=O)[C@H](CC(=O)O)NC(=O)[C@H](Cc4ccc(O)cc4)NC(=O)CN(C)C3=O)cc2)cc1]

Example 193 was prepared on a 50 umol scale. It was purified by Preparative LC/MS. The yield of the product was 25.8 mg, and its estimated purity by LCMS analysis was 100%. Analysis condition 2: Retention time=1.47 min. ESI-MS(+) m/z [M+H]⁺: 1906.

### Preparation of Example 194

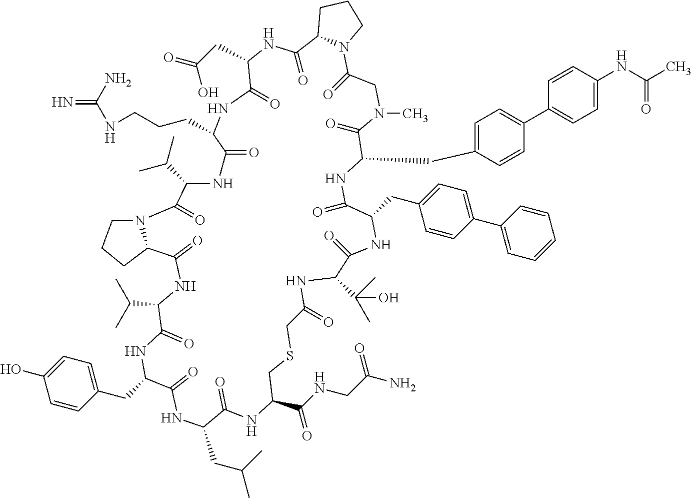 [SMILES: CC(=O)Nc1ccc(-c2ccc(C[C@@H]3NC(=O)[C@H](Cc4ccc(-c5ccccc5)cc4)NC(=O)[C@H](C(C)(C)O)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](C(C)C)NC(=O)[C@H](CCCNC(=N)N)NC(=O)[C@H](CC(=O)O)NC(=O)[C@@H]4CCCN4C(=O)CN(C)C3=O)cc2)cc1]

Example 194 was prepared on a 50 Preparative LC/MS. The yield of the product was 28.9 mg, and its estimated purity by LCMS analysis was 98.2%. Analysis condition 1: Retention time=1.43 min. ESI-MS(+) m/z [M+H]⁺: 1847.1.

### Preparation of Example 195

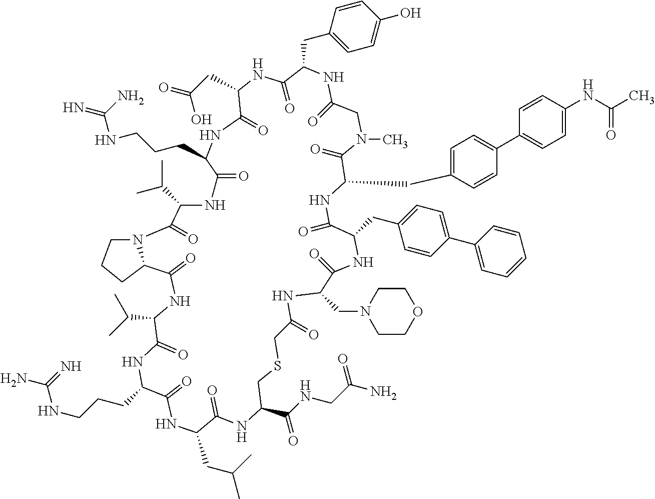 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccc(O)cc4)NC(=O)CN(C)C3=O)cc2)cc1]

Example 195 was prepared on a 50 umol scale. It was purified by Preparative LC/MS. The yield of the product was 25.8 mg, and its estimated purity by LCMS analysis was 100%. Analysis condition 1: Retention time=1.42 min. ESI-MS(+) m/z [M+H]⁺: 1948.3.

### Preparation of Example 196

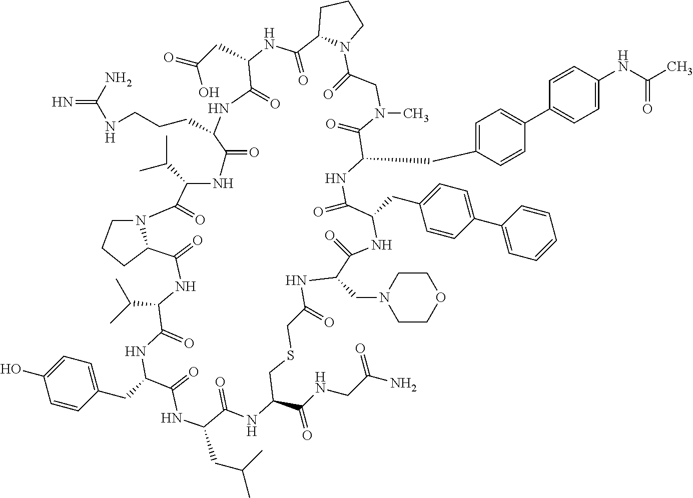 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](C(C)C)NC(=O)[C@H](CCCNC(=N)N)NC(=O)[C@H](CC(=O)O)NC(=O)[C@@H]4CCCN4C(=O)CN(C)C3=O)cc2)cc1]

Example 196 was prepared on a 50 Preparative LC/MS. The yield of the product was 29 mg, and its estimated purity by LCMS analysis was 98.5%. Analysis condition 2: Retention time=1.41 min. ESI-MS(+) m/z [M+H]⁺: 1888.2.

### Preparation of Example 197

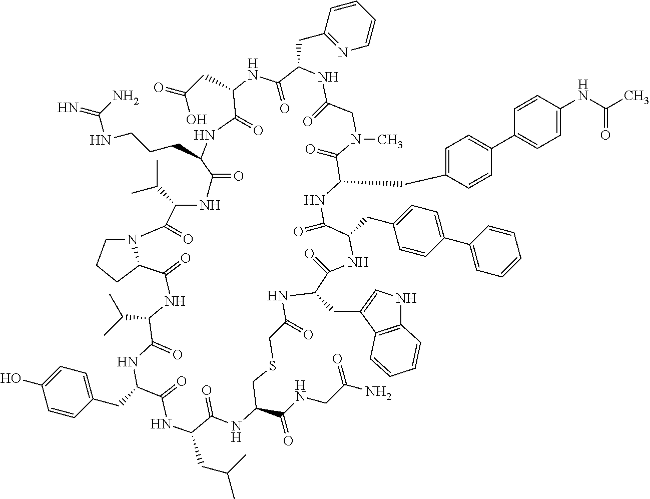 [SMILES: CC(=O)Nc1ccc(-c2ccc(C[C@@H]3NC(=O)[C@H](Cc4ccc(-c5ccccc5)cc4)NC(=O)[C@H](Cc4c[nH]c5ccccc45)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 197 was prepared on a 50 umol scale. It was purified by Preparative LC/MS. The yield of the product was 25.5 mg, and its estimated purity by LCMS analysis was 100%. Analysis condition 1: Retention time=1.43 min. ESI-MS(+) m/z [M+H]⁺: 1969.

### Preparation of Example 198

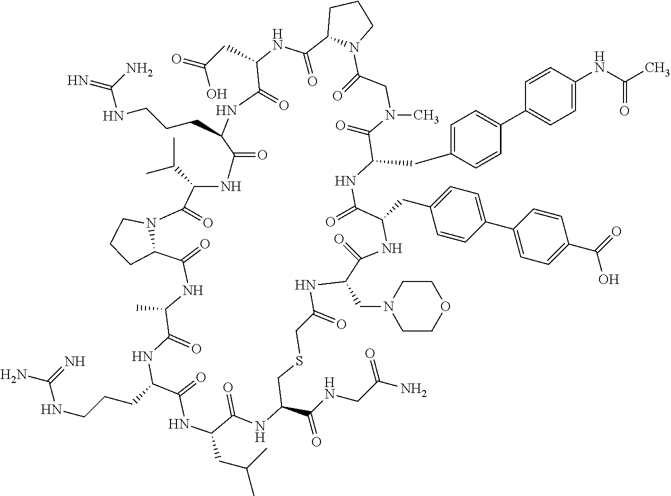 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](CCCNC(=N)N)NC(=O)[C@H](C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 198 was prepared on a 50 Preparative LC/MS. The yield of the product was 20.7 mg, and its estimated purity by LCMS analysis was 99.3%. Analysis condition 1: Retention time=1.08 min. ESI-MS(+) m/z [M+H]⁺: 1897.1.

### Preparation of Example 199

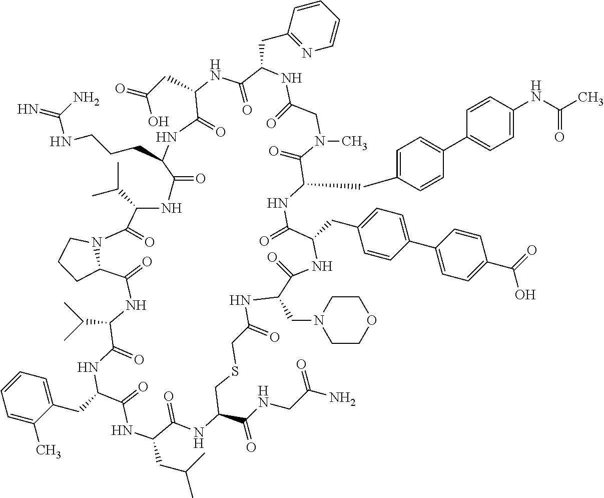 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccccc4C)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 199 was prepared on a 50 umol scale. It was purified by Preparative LC/MS. The yield of the product was 31.8 mg, and its estimated purity by LCMS analysis was 96.3%. Analysis condition 1: Retention time=1.31 min. ESI-MS(+) m/z [M+H]⁺: 1981.

### Preparation of Example 200

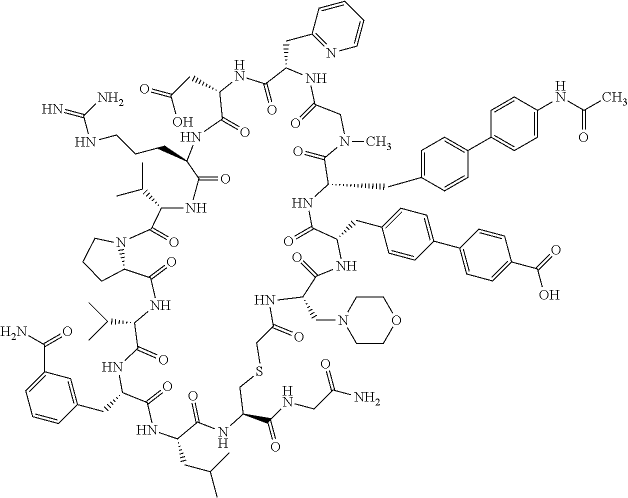 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cccc(C(N)=O)c4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 200 was prepared on a 50 Preparative LC/MS. The yield of the product was 33.1 mg, and its estimated purity by LCMS analysis was 97%. Analysis condition 1: Retention time=1.14 min. ESI-MS(+) m/z [M+2H] 2+: 1006.

### Preparation of Example 201

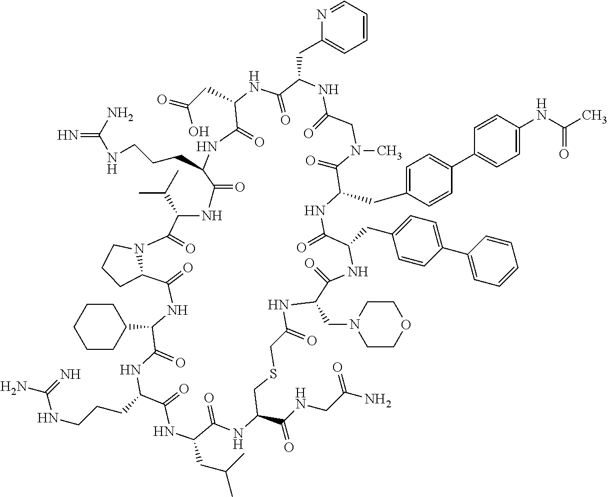 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](CCCNC(=N)N)NC(=O)[C@H](C4CCCCC4)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 201 was prepared on a 50 umol scale. It was purified by Preparative LC/MS. The yield of the product was 19.6 mg, and its estimated purity by LCMS analysis was 96.6%. Analysis condition 1: Retention time=1.55 min. ESI-MS (+) m/z [M+H]⁺: 1971.8.

### Preparation of Example 202

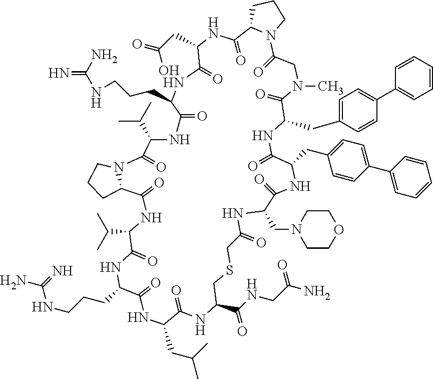 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 202 was prepared on a 50 umol scale. It was purified by Preparative LC/MS. The yield of the product was 31.1 mg, and its estimated purity by LCMS analysis was 98.2%. Analysis condition 2: Retention time=1.48 min. ESI-MS(+) m/z [M+H]⁺: 1825.

### Preparation of Example 203

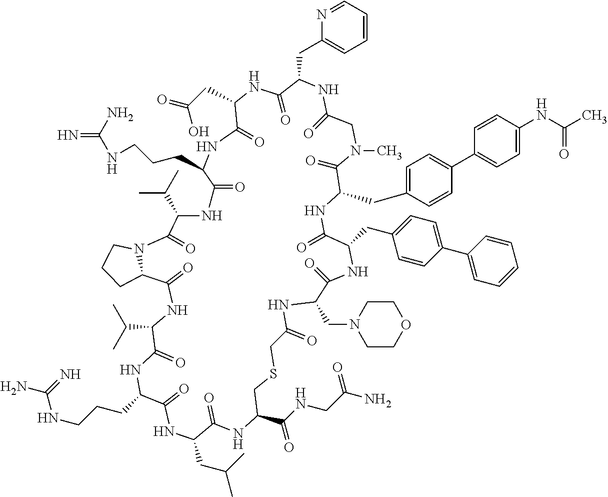 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 203 was prepared on a 50 umol scale. It was purified by Preparative LC/MS. The yield of the product was 24.1 mg, and its estimated purity by LCMS analysis was 95%. Analysis condition 2: Retention time=1.26 min. ESI-MS (+) m/z [M+2H]²⁺: 967.2.

### Preparation of Example 204

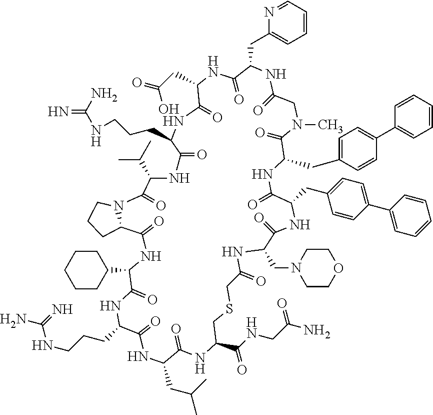 [SMILES: CC(C)C[C@@H]1NC(=O)[C@H](CCCNC(=N)N)NC(=O)[C@H](C2CCCCC2)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 204 was prepared on a 50 umol scale. It was purified by Preparative LC/MS. The yield of the product was 3.8 mg, and its estimated purity by LCMS analysis was 96.8%. Analysis condition 2: Retention time=1.48 min. ESI-MS(+) m/z [M+H]⁺: 1916.2.

### Preparation of Example 205

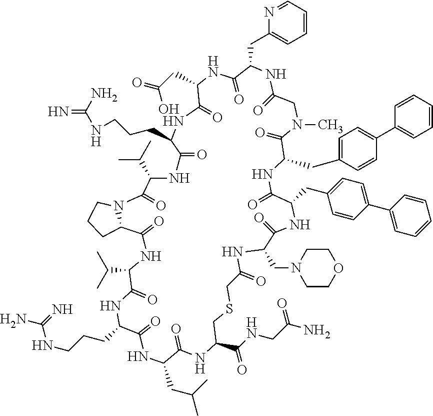 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 205 was prepared on a 50 umol scale. It was purified by Preparative LC/MS. The yield of the product was 10.8 mg, and its estimated purity by LCMS analysis was 100%. Analysis condition 2: Retention time=1.42 min. ESI-MS(+) m/z [M+2H]²⁺: 938.2.

### Preparation of Example 206

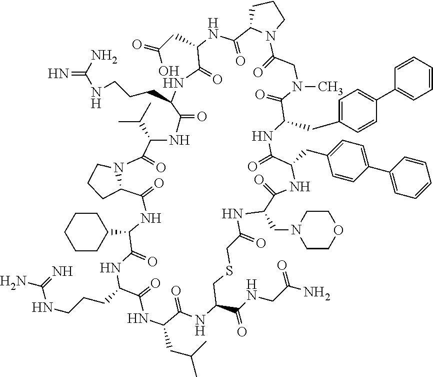 [SMILES: CC(C)C[C@@H]1NC(=O)[C@H](CCCNC(=N)N)NC(=O)[C@H](C2CCCCC2)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 206 was prepared on a 50 umol scale. It was purified by Preparative LC/MS. The yield of the product was 22.2 mg, and its estimated purity by LCMS analysis was 96.6%. Analysis condition 1: Retention time=1.57 min. ESI-MS(+) m/z [M+H]⁺: 1865.1.

### Preparation of Example 207

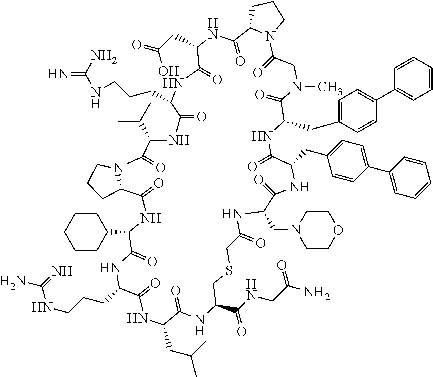 [SMILES: CC(C)C[C@@H]1NC(=O)[C@H](CCCNC(=N)N)NC(=O)[C@H](C2CCCCC2)NC(=O)[C@@H]2CCCN2C(=O)[C@H](C(C)C)NC(=O)[C@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 207 was prepared on a 50 umol scale. It was purified by Preparative LC/MS. The yield of the product was 19.5 mg, and its estimated purity by LCMS analysis was 91.5%. Analysis condition 1: Retention time=1.74 min. ESI-MS(+) m/z [M+H]⁺: 1864.2.

### Preparation of Example 208

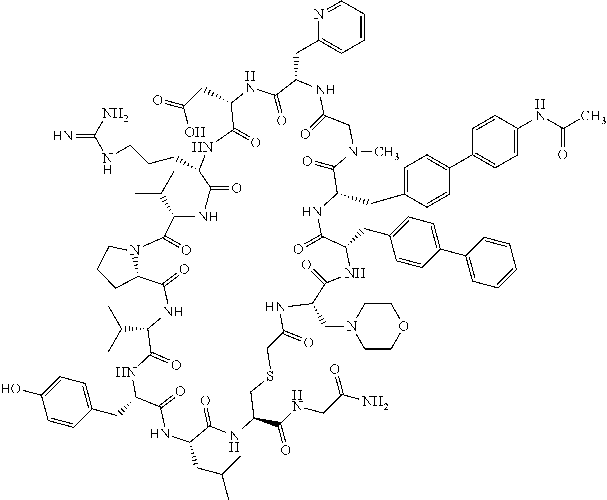 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](C(C)C)NC(=O)[C@H](CCCNC(=N)N)NC(=O)[C@H](CC(=O)O)NC(=O)[C@H](Cc4ccccn4)NC(=O)CN(C)C3=O)cc2)cc1]

Example 208 was prepared on a 50 umol scale. It was purified by Preparative LC/MS. The yield of the product was 8.1 mg, and its estimated purity by LCMS analysis was 100%. Analysis condition 2: Retention time=1.39 min. ESI-MS (+) m/z [M+H]⁺: 1938.9.

### Preparation of Example 209

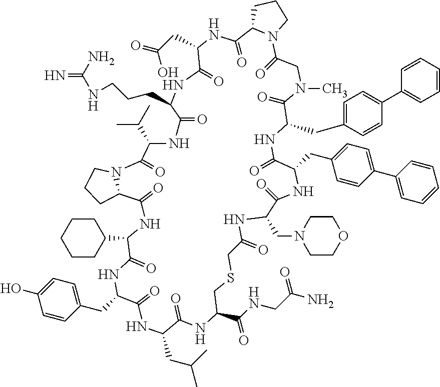 [SMILES: CC(C)C[C@@H]1NC(=O)[C@H](Cc2ccc(O)cc2)NC(=O)[C@H](C2CCCCC2)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 209 was prepared on a 50 umol scale. It was purified by Preparative LC/MS. The yield of the product was 25.8 mg, and its estimated purity by LCMS analysis was 100%. Analysis condition 1: Retention time=1.6 min. ESI-MS(+) m/z [M+2H]²⁺: 936.2.

### Preparation of Example 210

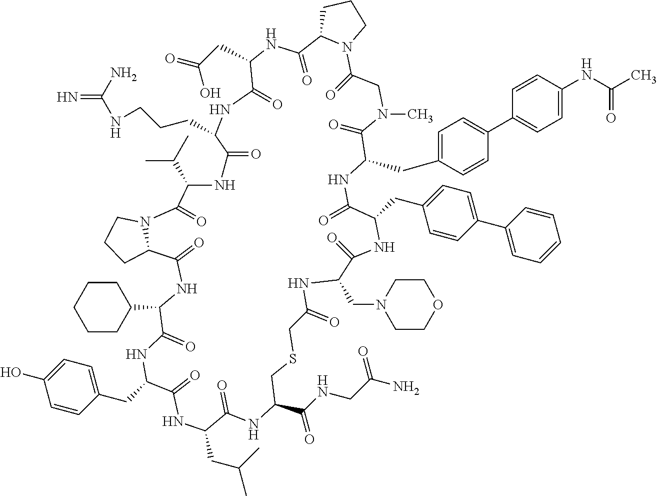 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccc(O)cc4)NC(=O)[C@H](C4CCCCC4)NC(=O)[C@@H]4CCCN4C(=O)[C@H](C(C)C)NC(=O)[C@H](CCCNC(=N)N)NC(=O)[C@H](CC(=O)O)NC(=O)[C@@H]4CCCN4C(=O)CN(C)C3=O)cc2)cc1]

Example 210 was prepared on a 50 umol scale. It was purified by Preparative LC/MS. The yield of the product was 19.6 mg, and its estimated purity by LCMS analysis was 97.8%. Analysis condition 2: Retention time=1.59 min. ESI-MS (+) m/z [M+H]⁺: 1928.

### Preparation of Example 211

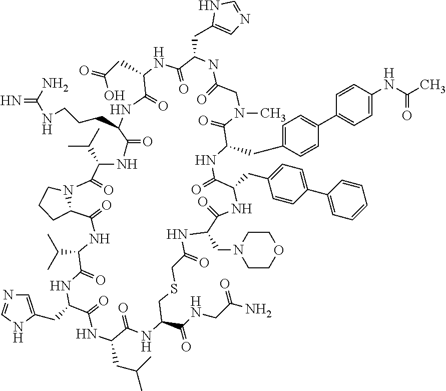 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cnc[nH]4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4cnc[nH]4)NC(=O)CN(C)C3=O)cc2)cc1]

Example 211 was prepared on a 50 Preparative LC/MS. The yield of the product was 14.4 mg, and its estimated purity by LCMS analysis was 97.9%. Analysis condition 2: Retention time=1.26 min. ESI-MS (+) m/z [M+H]⁺: 1901.9.

### Preparation of Example 212

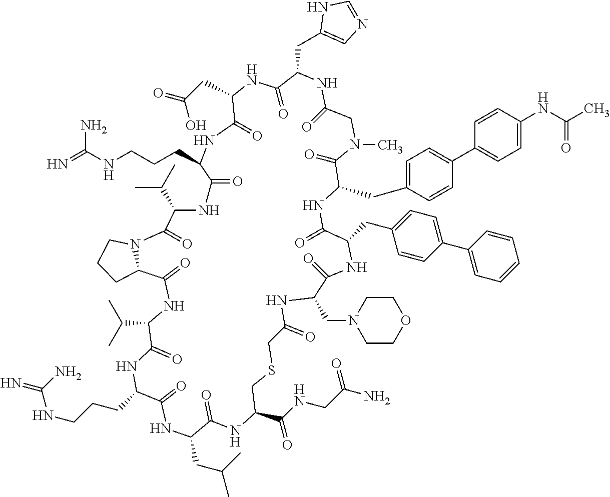 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4cnc[nH]4)NC(=O)CN(C)C3=O)cc2)cc1]

Example 212 was prepared on a 50 umol scale. It was purified by Preparative LC/MS. The yield of the product was 28 mg, and its estimated purity by LCMS analysis was 97.1%. Analysis condition 2: Retention time=1.23 min. ESI-MS(+) m/z [M+H]⁺: 1922.1.

### Preparation of Example 213

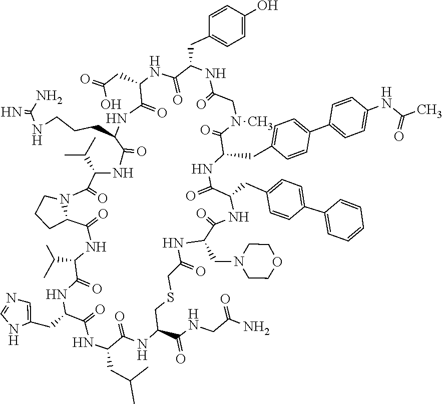 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cnc[nH]4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccc(O)cc4)NC(=O)CN(C)C3=O)cc2)cc1]

Example 213 was prepared on a 50 Preparative LC/MS. The yield of the product was 19.9 mg, and its estimated purity by LCMS analysis was 98.5%. Analysis condition 2: Retention time=1.35 min. ESI-MS (+) m/z [M+H]⁺: 1928.1.

### Preparation of Example 214

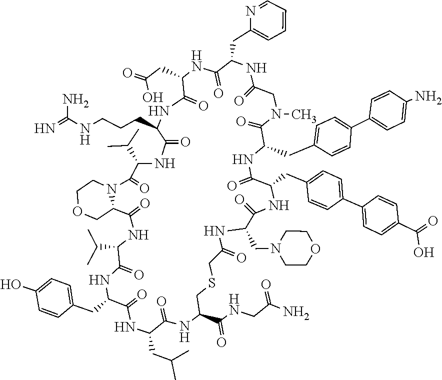 [SMILES: CC(C)C[C@@H]1NC(=O)[C@H](Cc2ccc(O)cc2)NC(=O)[C@H](C(C)C)NC(=O)[C@@H]2COCCN2C(=O)[C@H](C(C)C)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(N)cc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC1=O]

Example 214 was prepared on a 50 umol scale. It was purified by Preparative LC/MS. The yield of the product was 14.1 mg, and its estimated purity by LCMS analysis was 98.3%. Analysis condition 2: Retention time=1.28 min. ESI-MS (+) m/z [M+H]⁺: 1997.1.

### Preparation of Example 215

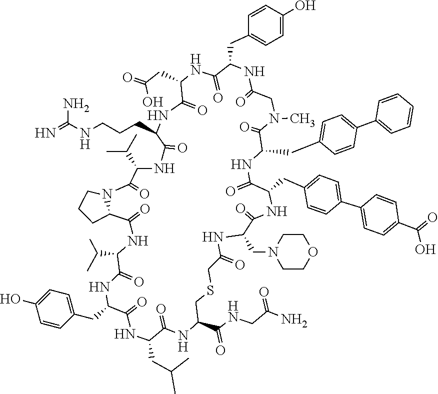 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC1=O]

Example 215 was prepared on a 25 umol scale. It was purified by Preparative LC/MS. The yield of the product was 12 mg, and its estimated purity by LCMS analysis was 97.6%. Analysis condition 1: Retention time=1.37 min. ESI-MS(+) m/z [M+H]⁺: 1969.1.

### Preparation of Example 216

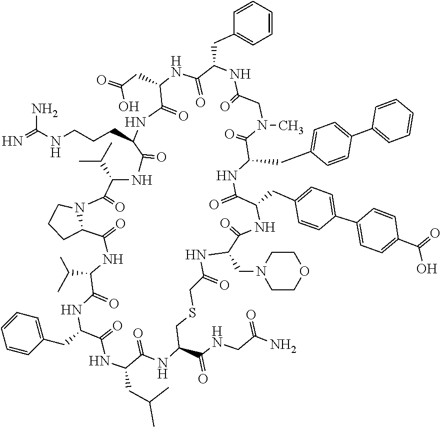 [SMILES: CC(C)C[C@@H]1NC(=O)[C@H](Cc2ccccc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccc2)NC(=O)CN(C)C(=O)[C@H](Cc2ccc(-c3ccccc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC1=O]

Example 216 was prepared on a 50 umol scale. It was purified by Preparative LC/MS. The yield of the product was 31.6 mg, and its estimated purity by LCMS analysis was 98.8%. Analysis condition 1: Retention time=1.56 min. ESI-MS (+) m/z [M+H]⁺: 1910.1.

### Preparation of Example 217

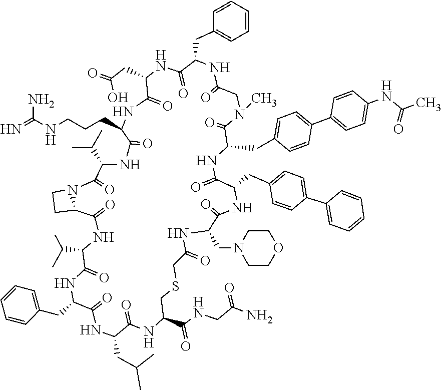 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccccc4)NC(=O)[C@H](C(C)C)NC(=O)[C@@H]4CCN4C(=O)[C@H](C(C)C)NC(=O)[C@@H](CCCNC(=N)N)NC(=O)[C@H](CC(=O)O)NC(=O)[C@H](Cc4ccccc4)NC(=O)CN(C)C3=O)cc2)cc1]

Example 217 was prepared on a 50 Preparative LC/MS. The yield of the product was 38.5 mg, and its estimated purity by LCMS analysis was 96%. Analysis condition 1: Retention time=1.58 min. ESI-MS (+) m/z [M+2H]²⁺: 955.2.

### Preparation of Example 218

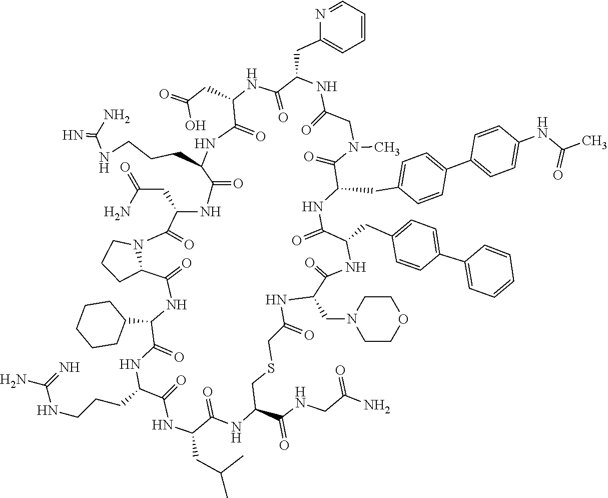 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](CCCNC(=N)N)NC(=O)[C@H](C4CCCCC4)NC(=O)[C@@H]4CCCN4C(=O)[C@H](CC(N)=O)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 218 was prepared on a 50 umol scale. It was purified by Preparative LC/MS. The yield of the product was 25.1 mg, and its estimated purity by LCMS analysis was 97.6%. Analysis condition 1: Retention time=1.47 min. ESI-MS (+) m/z [M+H]⁺: 1988.1.

### Preparation of Example 219

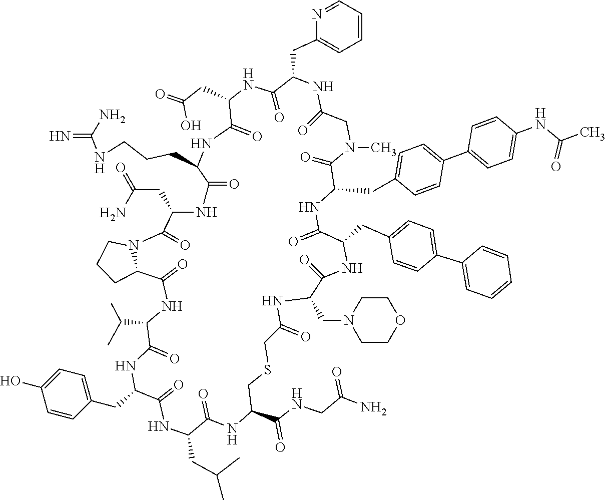 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](CC(N)=O)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 219 was prepared on a 50 Preparative LC/MS. The yield of the product was 35.6 mg, and its estimated purity by LCMS analysis was 98.7%. Analysis condition 1: Retention time=1.53 min. ESI-MS (+) m/z [M+H]⁺: 1994.1.

### Preparation of Example 220

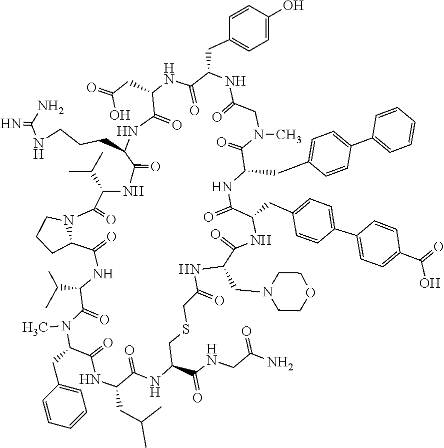 [SMILES: CC(C)C[C@@H]1NC(=O)[C@H](Cc2ccccc2)N(C)C(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccccc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC1=O]

Example 220 was prepared on a 50 umol scale. It was purified by Preparative LC/MS. The yield of the product was 16.8 mg, and its estimated purity by LCMS analysis was 95.8%. Analysis condition 2: Retention time=1.61 min. ESI-MS(+) m/z [M+H]⁺: 1939.

### Preparation of Example 221

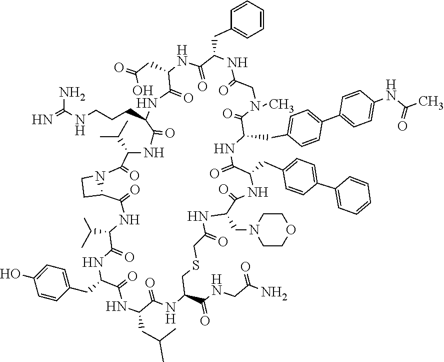 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCN4C(=O)[C@H](C(C)C)NC(=O)[C@@H](CCCNC(=N)N)NC(=O)[C@H](CC(=O)O)NC(=O)[C@H](Cc4ccccc4)NC(=O)CN(C)C3=O)cc2)cc1]

Example 221 was prepared on a 50 Preparative LC/MS. The yield of the product was 21.4 mg, and its estimated purity by LCMS analysis was 100%. Analysis condition 1: Retention time=1.6 min. ESI-MS (+) m/z [M+H]⁺: 1924.2.

### Preparation of Example 222

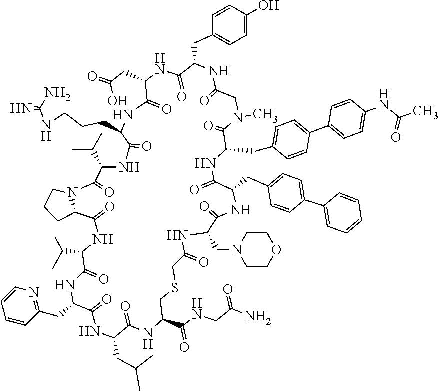 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccccn4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccc(O)cc4)NC(=O)CN(C)C3=O)cc2)cc1]

Example 222 was prepared on a 50 umol scale. It was purified by Preparative LC/MS. The yield of the product was 18.1 mg, and its estimated purity by LCMS analysis was 95.2%. Analysis condition 1: Retention time=1.53 min. ESI-MS (+) m/z [M+H]⁺: 1939.1.

### Preparation of Example 223

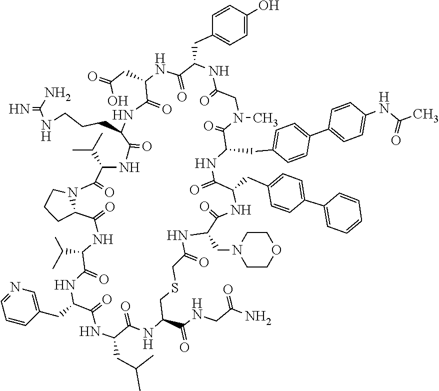 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cccnc4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccc(O)cc4)NC(=O)CN(C)C3=O)cc2)cc1]

Example 223 was prepared on a 50 Preparative LC/MS. The yield of the product was 29.9 mg, and its estimated purity by LCMS analysis was 96.9%. Analysis condition 2: Retention time=1.26 min. ESI-MS (+) m/z [M+2H]²⁺: 970.4.

### Preparation of Example 224

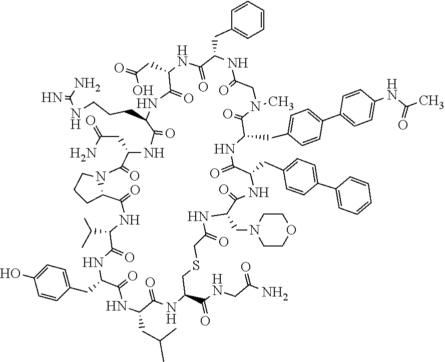 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](CC(N)=O)NC(=O)[C@@H](CCCNC(=N)N)NC(=O)[C@H](CC(=O)O)NC(=O)[C@H](Cc4ccccc4)NC(=O)CN(C)C3=O)cc2)cc1]

Example 224 was prepared on a 50 umol scale. It was purified by Preparative LC/MS. The yield of the product was 35 mg, and its estimated purity by LCMS analysis was 98.1%. Analysis condition 1: Retention time=1.37 min. ESI-MS(+) m/z [M+H]⁺: 1939.

### Preparation of Example 225

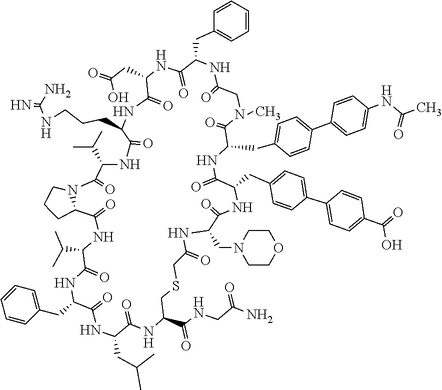 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccccc4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccc4)NC(=O)CN(C)C3=O)cc2)cc1]

Example 225 was prepared on a 50 Preparative LC/MS. The yield of the product was 26.7 mg, and its estimated purity by LCMS analysis was 97.4%. Analysis condition 1: Retention time=1.24 min. ESI-MS (+) m/z [M+H]⁺: 1966.1.

### Preparation of Example 226

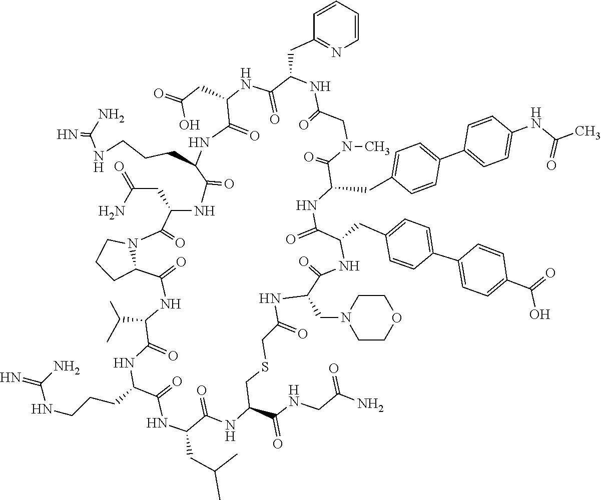 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]4CCCN4C(=O)[C@H](CC(N)=O)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 226 was prepared on a 50 umol scale. It was purified by Preparative LC/MS. The yield of the product was 20.5 mg, and its estimated purity by LCMS analysis was 95.1%. Analysis condition 2: Retention time=1.16 min. ESI-MS (+) m/z [M+2H]²⁺: 996.1.

### Preparation of Example 227

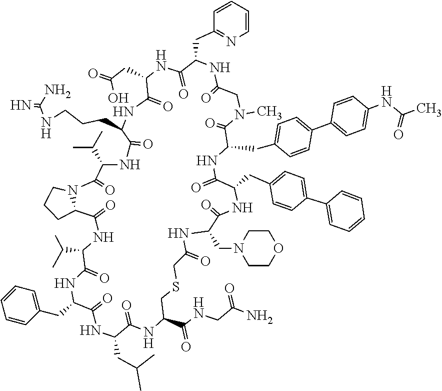 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccccc4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 227 was prepared on a 50 Preparative LC/MS. The yield of the product was 23 mg, and its estimated purity by LCMS analysis was 95.5%. Analysis condition 2: Retention time=1.31 min. ESI-MS(+) m/z [M+3H]³⁺: 642.1.

### Preparation of Example 228

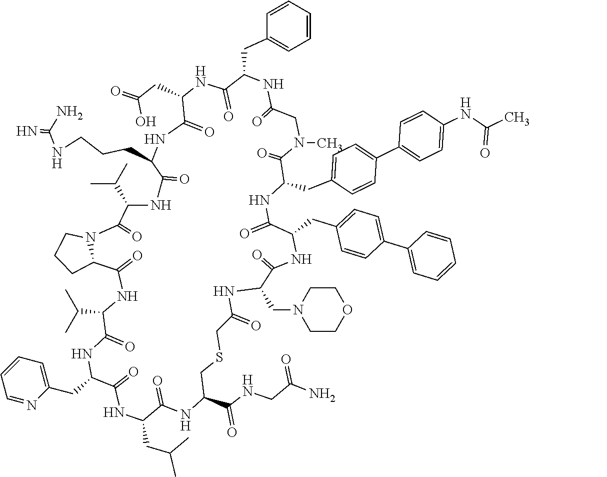 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccccn4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccc4)NC(=O)CN(C)C3=O)cc2)cc1]

Example 228 was prepared on a 50 umol scale. It was purified by Preparative LC/MS. The yield of the product was 13.5 mg, and its estimated purity by LCMS analysis was 96%. Analysis condition 2: Retention time=1.31 min. ESI-MS (+) m/z [M+3H]³⁺: 642.

### Preparation of Example 229

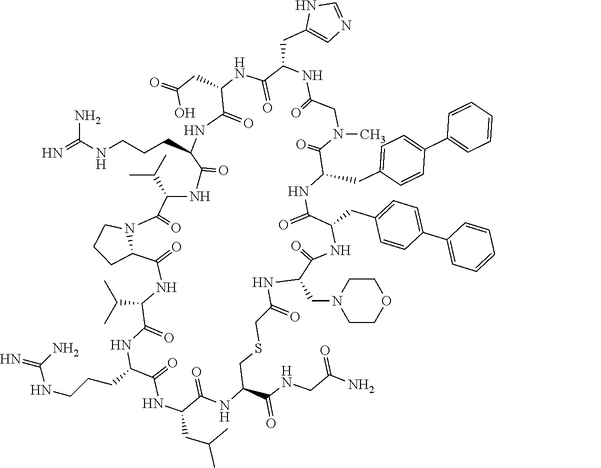 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 229 was prepared on a 50 umol scale. It was purified by Preparative LC/MS. The yield of the product was 10.9 mg, and its estimated purity by LCMS analysis was 99.2%. Analysis condition 2: Retention time=1.31 min. ESI-MS(+) m/z [M+2H]²⁺: 933.1.

### Preparation of Example 230

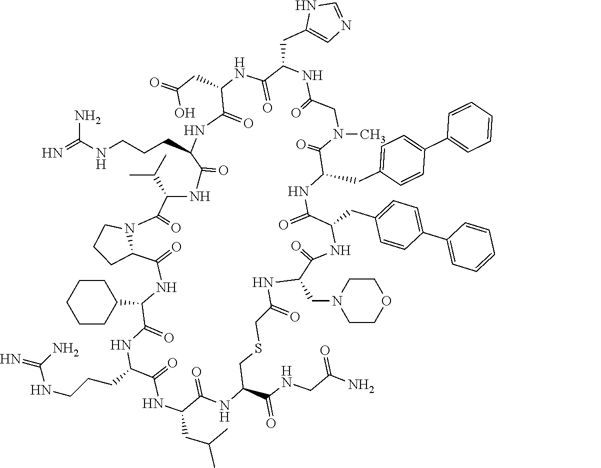 [SMILES: CC(C)C[C@@H]1NC(=O)[C@H](CCCNC(=N)N)NC(=O)[C@H](C2CCCCC2)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 230 was prepared on a 50 umol scale. It was purified by Preparative LC/MS. The yield of the product was 15.6 mg, and its estimated purity by LCMS analysis was 100%. Analysis condition 1: Retention time=1.62 min. ESI-MS(+) m/z [M+H]⁺: 953.2.

### Preparation of Example 231

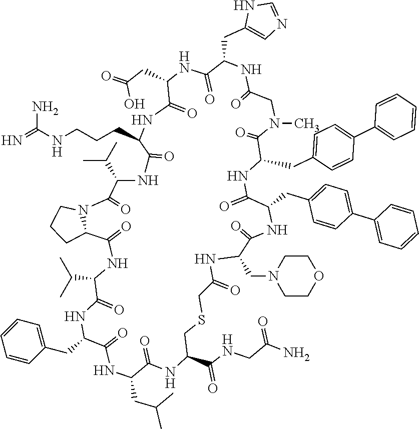 [SMILES: CC(C)C[C@@H]1NC(=O)[C@H](Cc2ccccc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccccc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 231 was prepared on a 50 umol scale. It was purified by Preparative LC/MS. The yield of the product was 6.2 mg, and its estimated purity by LCMS analysis was 96%. Analysis condition 1: Retention time=1.64 min. ESI-MS(+) m/z [M+H]⁺: 1855.1.

### Preparation of Example 232

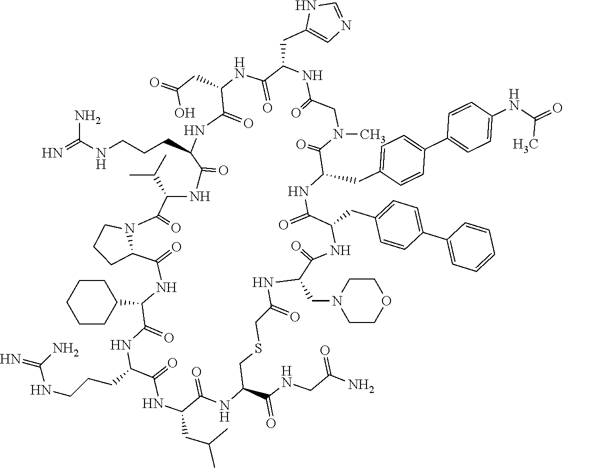 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](CCCNC(=N)N)NC(=O)[C@H](C4CCCCC4)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4cnc[nH]4)NC(=O)CN(C)C3=O)cc2)cc1]

Example 232 was prepared on a 50 umol scale. It was purified by Preparative LC/MS. The yield of the product was 13.8 mg, and its estimated purity by LCMS analysis was 96.3%. Analysis condition 1: Retention time=1.42 min. ESI-MS (+) m/z [M+2H]²⁺: 981.3.

### Preparation of Example 233

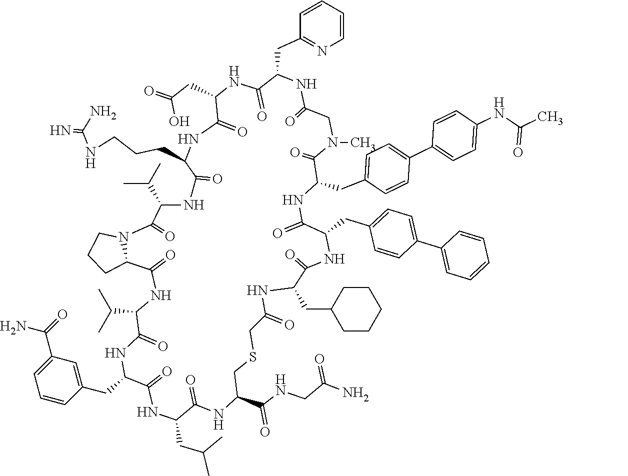 [SMILES: CC(=O)Nc1ccc(-c2ccc(C[C@@H]3NC(=O)[C@H](Cc4ccc(-c5ccccc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4cccc(C(N)=O)c4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 233 was prepared on a 50 Preparative LC/MS. The yield of the product was 10.5 mg, and its estimated purity by LCMS analysis was 100%. Analysis condition 1: Retention time=1.55 min. ESI-MS (+) m/z [M+H]⁺: 1963.2.

### Preparation of Example 234

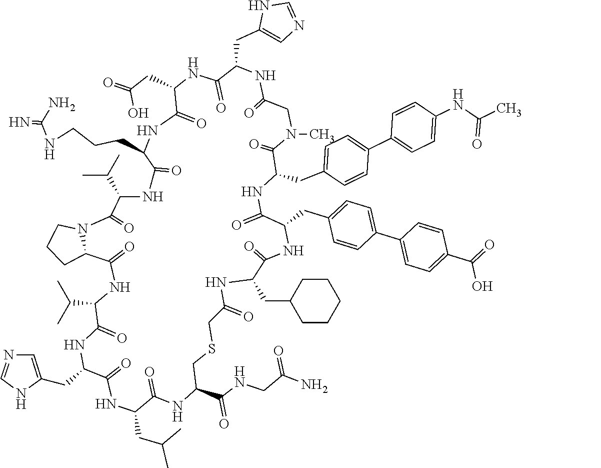 [SMILES: CC(=O)Nc1ccc(-c2ccc(C[C@@H]3NC(=O)[C@H](Cc4ccc(-c5ccc(C(=O)O)cc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4cnc[nH]4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4cnc[nH]4)NC(=O)CN(C)C3=O)cc2)cc1]

Example 234 was prepared on a 50 umol scale. It was purified by Preparative LC/MS. The yield of the product was 3.7 mg, and its estimated purity by LCMS analysis was 100%. Analysis condition 1: Retention time=1.19 min. ESI-MS (+) m/z [M+H]⁺: 1943.4.

### Preparation of Example 235

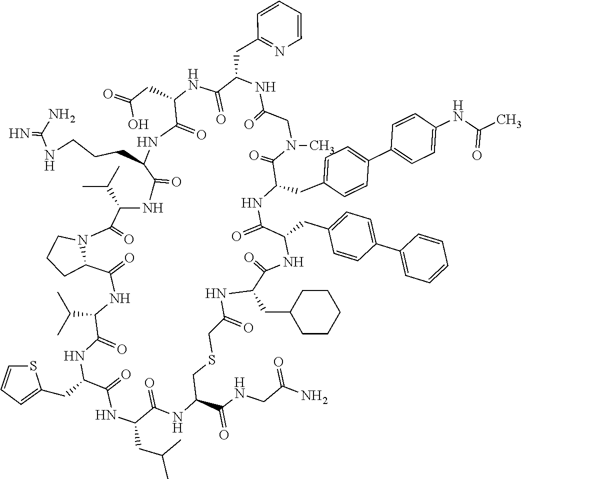 [SMILES: CC(=O)Nc1ccc(-c2ccc(C[C@@H]3NC(=O)[C@H](Cc4ccc(-c5ccccc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4cccs4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 235 was prepared on a 50 Preparative LC/MS. The yield of the product was 14 mg, and its estimated purity by LCMS analysis was 93.8%. Analysis condition 2: Retention time=1.69 min. ESI-MS(+) m/z [M+2H]²⁺: 963.9.

### Preparation of Example 236

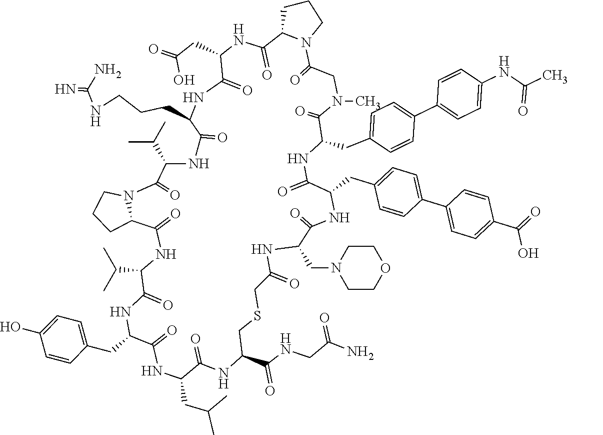 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 236 was prepared on a 50 umol scale. It was purified by Preparative LC/MS. The yield of the product was 21.8 mg, and its estimated purity by LCMS analysis was 94.6%. Analysis condition 1: Retention time=1.01 min. ESI-MS (+) m/z [M+2H]²⁺: 966.6.

### Preparation of Example 237

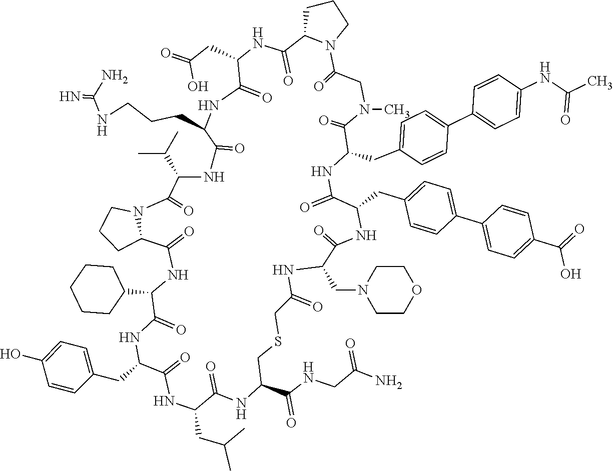 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccc(O)cc4)NC(=O)[C@H](C4CCCCC4)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 237 was prepared on a 50 Preparative LC/MS. The yield of the product was 25.3 mg, and its estimated purity by LCMS analysis was 91.1%. Analysis condition 1: Retention time=1.12 min. ESI-MS (+) m/z [M+2H]²⁺: 986.8.

### Preparation of Example 238

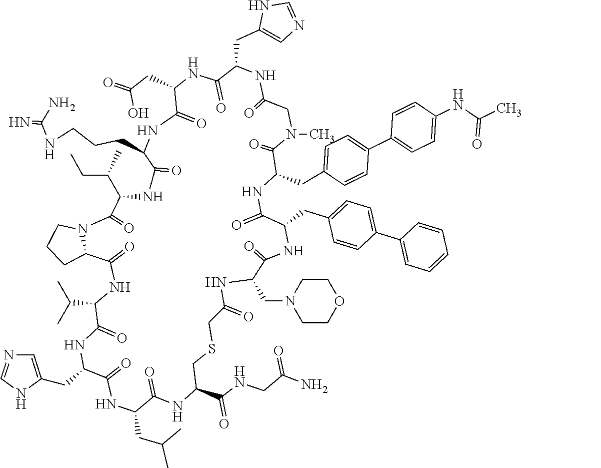 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(C)=O)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 238 was prepared on a 50 umol scale. It was purified by Preparative LC/MS 25. The yield of the product was 5.8 mg, and its estimated purity by LCMS analysis was 93.5%. Analysis condition 10: Retention time=1.33 min. ESI-MS (+) m/z [M+3H]³⁺: 639.6.

### Preparation of Example 239

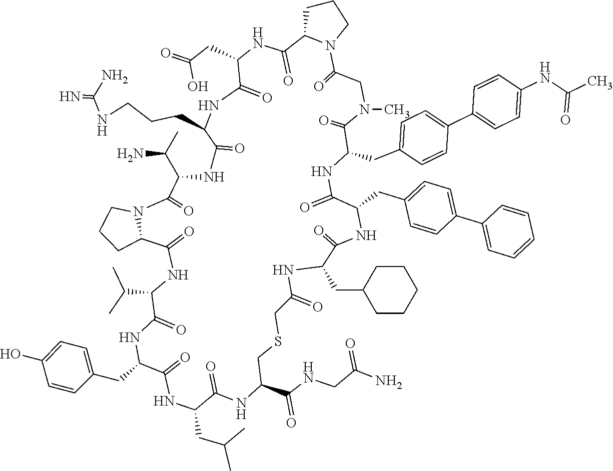 [SMILES: CC(=O)Nc1ccc(-c2ccc(C[C@@H]3NC(=O)[C@H](Cc4ccc(-c5ccccc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H]([C@H](C)N)NC(=O)[C@@H](CCCNC(=N)N)NC(=O)[C@H](CC(=O)O)NC(=O)[C@@H]4CCCN4C(=O)CN(C)C3=O)cc2)cc1]

Example 239 was prepared on a 50 Preparative LC/MS. The yield of the product was 22.8 mg, and its estimated purity by LCMS analysis was 98.2%. Analysis condition 1: Retention time=1.39 min. ESI-MS (+) m/z [M+2H]²⁺: 943.6.

### Preparation of Example 240

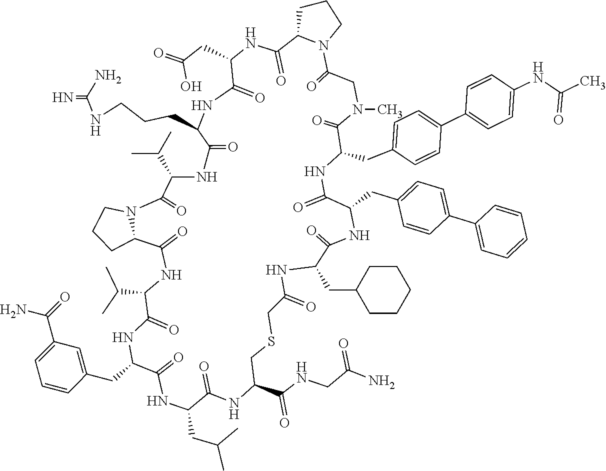 [SMILES: CC(=O)Nc1ccc(-c2ccc(C[C@@H]3NC(=O)[C@H](Cc4ccc(-c5ccccc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4cccc(C(N)=O)c4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 240 was prepared on a 50 umol scale. It was purified by Preparative LC/MS. The yield of the product was 19.9 mg, and its estimated purity by LCMS analysis was 100%. Analysis condition 1: Retention time=1.37 min. ESI-MS (+) m/z [M+H]⁺: 1911.9.

### Preparation of Example 241

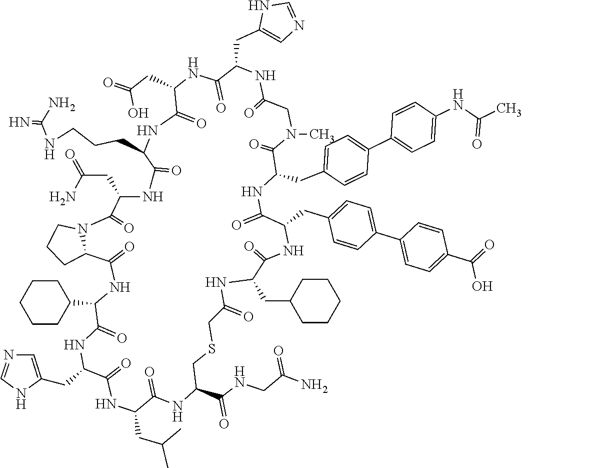 [SMILES: CC(=O)Nc1ccc(-c2ccc(C[C@@H]3NC(=O)[C@H](Cc4ccc(-c5ccc(C(=O)O)cc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4cnc[nH]4)NC(=O)[C@H](C4CCCCC4)NC(=O)[C@@H]4CCCN4C(=O)[C@H](CC(N)=O)NC(=O)[C@@H](CCCNC(=N)N)NC(=O)[C@H](CC(=O)O)NC(=O)[C@H](Cc4cnc[nH]4)NC(=O)CN(C)C3=O)cc2)cc1]

Example 241 was prepared on a 50 Preparative LC/MS. The yield of the product was 30.9 mg, and its estimated purity by LCMS analysis was 100%. Analysis condition 1: Retention time=1.21 min. ESI-MS (+) m/z [M+2H]²⁺: 1001.3.

### Preparation of Example 242

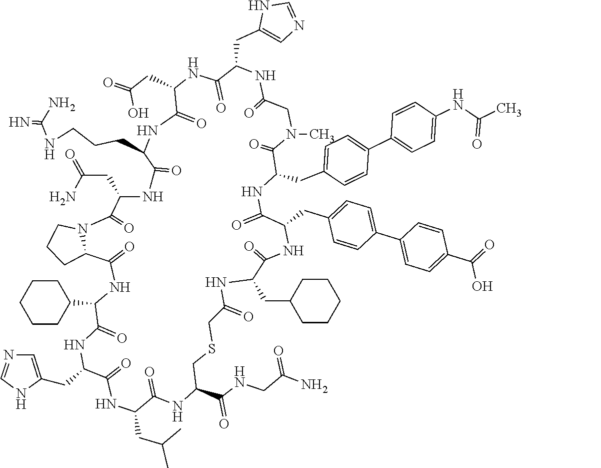 [SMILES: CC(=O)Nc1ccc(-c2ccc(C[C@@H]3NC(=O)[C@H](Cc4ccc(-c5ccc(C(=O)O)cc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4cnc[nH]4)NC(=O)[C@H](C4CCCCC4)NC(=O)[C@@H]4CCCN4C(=O)[C@H](CC(N)=O)NC(=O)[C@@H](CCCNC(=N)N)NC(=O)[C@H](CC(=O)O)NC(=O)[C@H](Cc4cnc[nH]4)NC(=O)CN(C)C3=O)cc2)cc1]

Example 242 was prepared on a 50 umol scale. It was purified by Preparative LC/MS. The yield of the product was 19.9 mg, and its estimated purity by LCMS analysis was 97.7%. Analysis condition 2: Retention time=1.31 min. ESI-MS (+) m/z [M+H]⁺: 1982.9.

### Preparation of Example 243

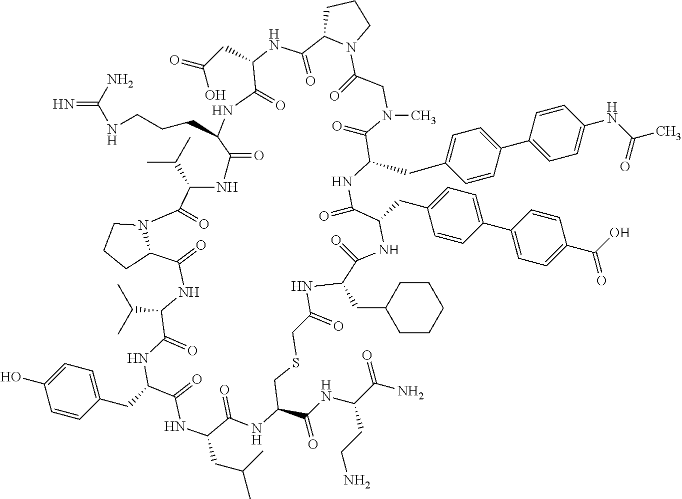 [SMILES: CC(=O)Nc1ccc(-c2ccc(C[C@@H]3NC(=O)[C@H](Cc4ccc(-c5ccc(C(=O)O)cc5)cc4)NC(=O)[C@H](CC4CCCCC4)NC(=O)CSC[C@@H](C(=O)N[C@@H](CCN)C(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 243 was prepared on a 50 Preparative LC/MS. The yield of the product was 11.4 mg, and its estimated purity by LCMS analysis was 96.3%. Analysis condition 2: Retention time=1.33 min. ESI-MS (+) m/z [M+H]⁺: 1971.5.

### Preparation of Example 244

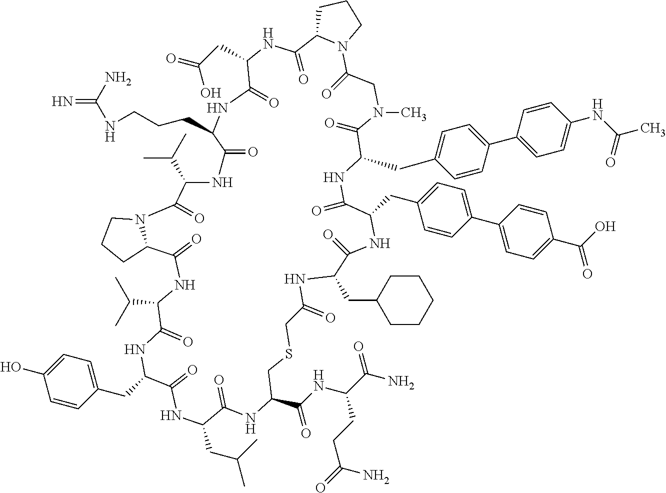 [SMILES: CC(=O)Nc1ccc(-c2ccc(C[C@@H]3NC(=O)[C@H](Cc4ccc(-c5ccc(C(=O)O)cc5)cc4)NC(=O)[C@H](CC4CCCCC4)NC(=O)CSC[C@@H](C(=O)N[C@@H](CCC(N)=O)C(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 244 was prepared on a 50 umol scale. It was purified by Preparative LC/MS. The yield of the product was 23.3 mg, and its estimated purity by LCMS analysis was 91.1%. Analysis condition 1: Retention time=1.21 min. ESI-MS (+) m/z [M+2H]²⁺: 1001.1.

### Preparation of Example 245

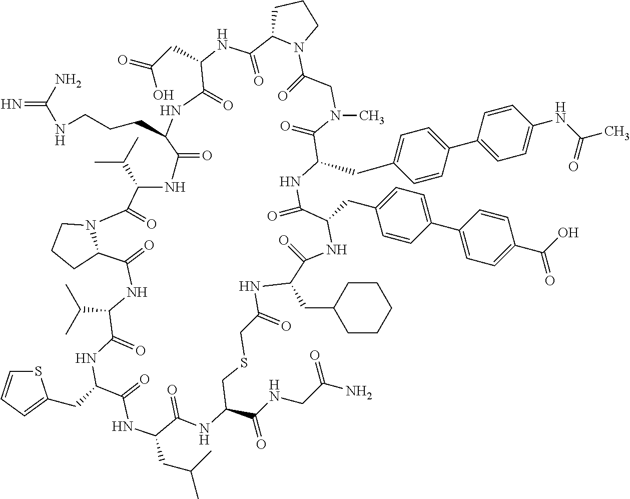 [SMILES: CC(=O)Nc1ccc(-c2ccc(C[C@@H]3NC(=O)[C@H](Cc4ccc(-c5ccc(C(=O)O)cc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4cccs4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 245 was prepared on a 50 Preparative LC/MS. The yield of the product was 27.4 mg, and its estimated purity by LCMS analysis was 90.4%. Analysis condition 2: Retention time=1.59 min. ESI-MS (+) m/z [M+2H]²⁺: 960.4.

### Preparation of Example 246

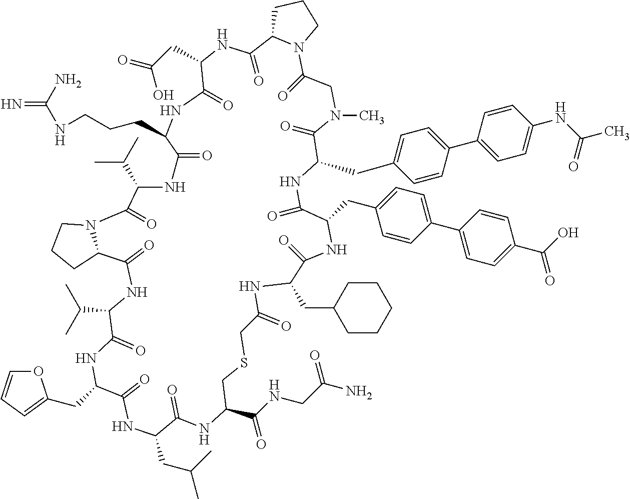 [SMILES: CC(=O)Nc1ccc(-c2ccc(C[C@@H]3NC(=O)[C@H](Cc4ccc(-c5ccc(C(=O)O)cc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4ccco4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 246 was prepared on a 50 umol scale. It was purified by Preparative LC/MS. The yield of the product was 21.3 mg, and its estimated purity by LCMS analysis was 90.1%. Analysis condition 1: Retention time=1.23 min. ESI-MS (+) m/z [M+H]⁺: 1902.8.

### Preparation of Example 247

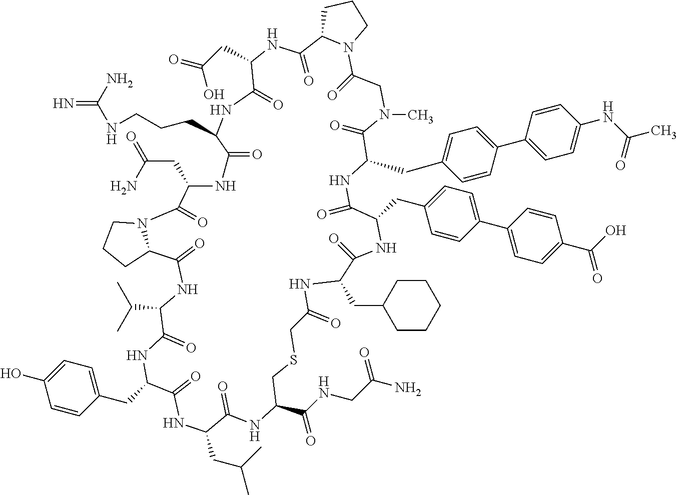 [SMILES: CC(=O)Nc1ccc(-c2ccc(C[C@@H]3NC(=O)[C@H](Cc4ccc(-c5ccc(C(=O)O)cc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](CC(N)=O)NC(=O)[C@@H](CCCNC(=N)N)NC(=O)[C@H](CC(=O)O)NC(=O)[C@@H]4CCCN4C(=O)CN(C)C3=O)cc2)cc1]

Example 247 was prepared on a 50 Preparative LC/MS. The yield of the product was 12.2 mg, and its estimated purity by LCMS analysis was 94%. Analysis condition 1: Retention time=1.15 min. ESI-MS (+) m/z [M+2H]²⁺: 972.9.

### Preparation of Example 248

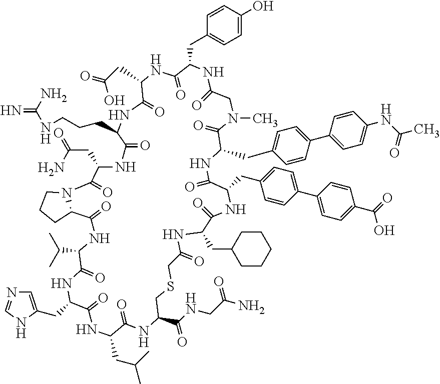 [SMILES: CC(=O)Nc1ccc(-c2ccc(C[C@@H]3NC(=O)[C@H](Cc4ccc(-c5ccc(C(=O)O)cc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4cnc[nH]4)NC(=O)[C@H](C(C)C)NC(=O)[C@@H]4CCCN4C(=O)[C@H](CC(N)=O)NC(=O)[C@@H](CCCNC(=N)N)NC(=O)[C@H](CC(=O)O)NC(=O)[C@H](Cc4ccc(O)cc4)NC(=O)CN(C)C3=O)cc2)cc1]

Example 248 was prepared on a 50 umol scale. It was purified by Preparative LC/MS. The yield of the product was 18.9 mg, and its estimated purity by LCMS analysis was 96.1%. Analysis condition 1: Retention time=1.24 min. ESI-MS (+) m/z [M+H]⁺: 1983.9.

### Preparation of Example 249

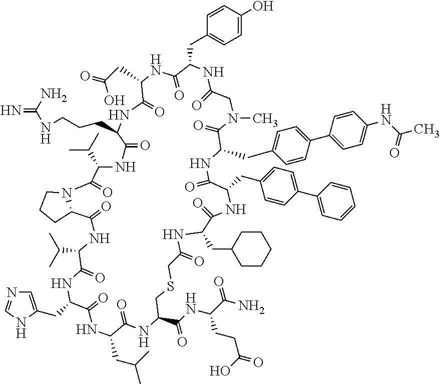 [SMILES: CC(=O)Nc1ccc(-c2ccc(C[C@@H]3NC(=O)[C@H](Cc4ccc(-c5ccccc5)cc4)NC(=O)[C@H](CC4CCCCC4)NC(=O)CSC[C@@H](C(=O)N[C@@H](CCC(=O)O)C(N)=O)NC(=O)[C@H](CC(C)C)NC(=O)[C@H](Cc4cnc[nH]4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccc(O)cc4)NC(=O)CN(C)C3=O)cc2)cc1]

Example 249 was prepared on a 50 umol scale. It was purified by Preparative LC/MS. The yield of the product was 13.7 mg, and its estimated purity by LCMS analysis was 95.2%. Analysis condition 1: Retention time=1.44 min. ESI-MS(+) m/z [M+H]⁺: 1996.7.

### Preparation of Example 250

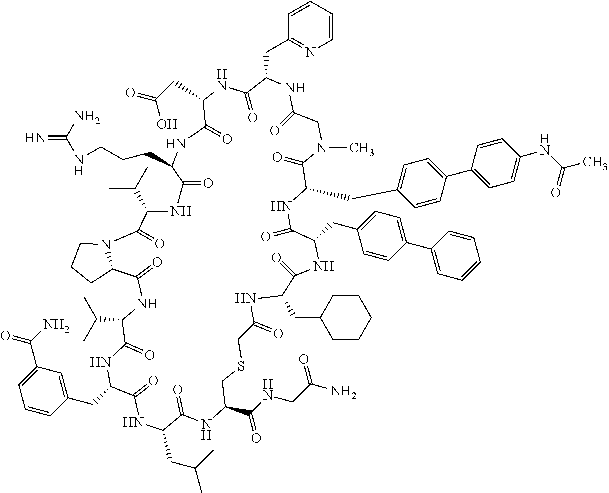 [SMILES: CC(=O)Nc1ccc(-c2ccc(C[C@@H]3NC(=O)[C@H](Cc4ccc(-c5ccccc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4cccc(C(N)=O)c4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 250 was prepared on a 50 umol scale. It was purified by Preparative LC/MS. The yield of the product was 7.2 mg, and its estimated purity by LCMS analysis was 100%. Analysis condition 2: Retention time=1.58 min. ESI-MS(+) m/z [M+H]⁺: 1977.

### Preparation of Example 251

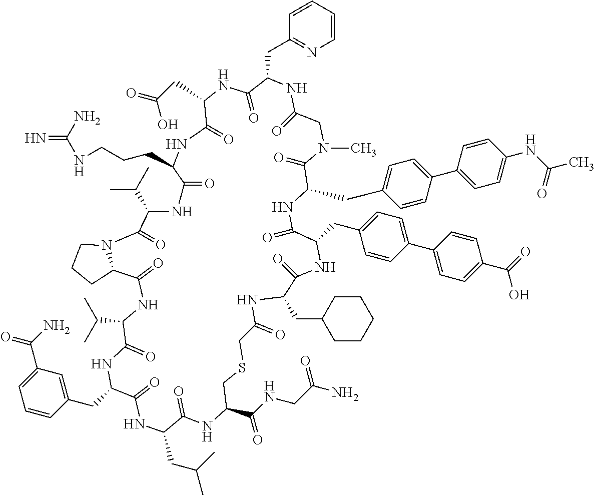 [SMILES: CC(=O)Nc1ccc(-c2ccc(C[C@@H]3NC(=O)[C@H](Cc4ccc(-c5ccc(C(=O)O)cc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4cccc(C(N)=O)c4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 251 was prepared on a 50 umol scale. It was purified by Preparative LC/MS. The yield of the product was 5.6 mg, and its estimated purity by LCMS analysis was 95.2%. Analysis condition 2: Retention time=1.37 min. ESI-MS(+) m/z [M+2H]²⁺: 1004.3.

### Preparation of Example 252

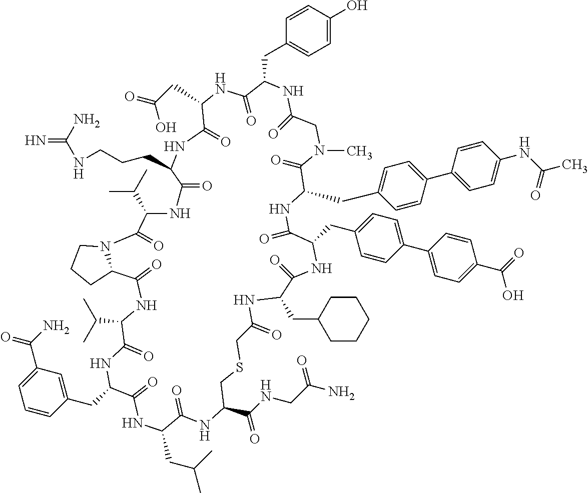 [SMILES: CC(=O)Nc1ccc(-c2ccc(C[C@@H]3NC(=O)[C@H](Cc4ccc(-c5ccc(C(=O)O)cc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4cccc(C(N)=O)c4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccc(O)cc4)NC(=O)CN(C)C3=O)cc2)cc1]

Example 252 was prepared on a 50 umol scale. It was purified by Preparative LC/MS. The yield of the product was 22.7 mg, and its estimated purity by LCMS analysis was 96.5%. Analysis condition 1: Retention time=1.26 min. ESI-MS(+) m/z [M+2H]²⁺: 1011.8.

### Preparation of Example 253

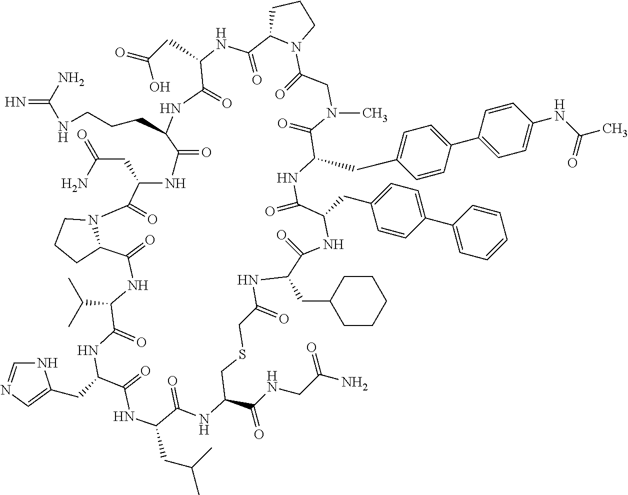 [SMILES: CC(=O)Nc1ccc(-c2ccc(C[C@@H]3NC(=O)[C@H](Cc4ccc(-c5ccccc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4cnc[nH]4)NC(=O)[C@H](C(C)C)NC(=O)[C@@H]4CCCN4C(=O)[C@H](CC(N)=O)NC(=O)[C@@H](CCCNC(=N)N)NC(=O)[C@H](CC(=O)O)NC(=O)[C@@H]4CCCN4C(=O)CN(C)C3=O)cc2)cc1]

Example 253 was prepared on a 50 umol scale. It was purified by Preparative LC/MS. The yield of the product was 23.2 mg, and its estimated purity by LCMS analysis was 95.5%. Analysis condition 2: Retention time=1.62 min. ESI-MS(+) m/z [M+2H]²⁺: 937.7.

### Preparation of Example 254

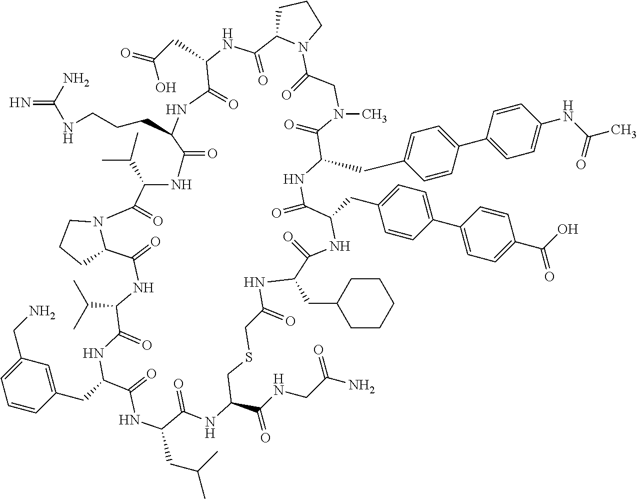 [SMILES: CC(=O)Nc1ccc(-c2ccc(C[C@@H]3NC(=O)[C@H](Cc4ccc(-c5ccc(C(=O)O)cc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4cccc(CN)c4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 254 was prepared on a 50 umol scale. It was purified by Preparative LC/MS. The yield of the product was 18.8 mg, and its estimated purity by LCMS analysis was 92.9%. Analysis condition 2: Retention time=1.29 min. ESI-MS(+) m/z [M+H]⁺: 1941.8.

### Preparation of Example 255

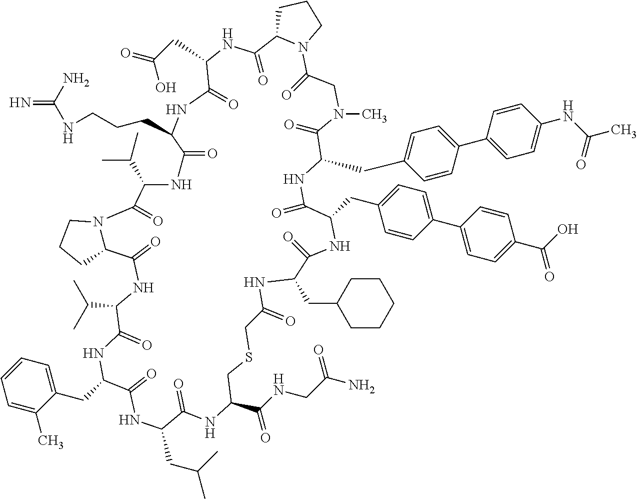 [SMILES: CC(=O)Nc1ccc(-c2ccc(C[C@@H]3NC(=O)[C@H](Cc4ccc(-c5ccc(C(=O)O)cc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4ccccc4C)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 255 was prepared on a 50 umol scale. It was purified by Preparative LC/MS. The yield of the product was 15.6 mg, and its estimated purity by LCMS analysis was 98.4%. Analysis condition 1: Retention time=1.32 min. ESI-MS(+) m/z [M+H]⁺: 1926.8.

### Preparation of Example 256

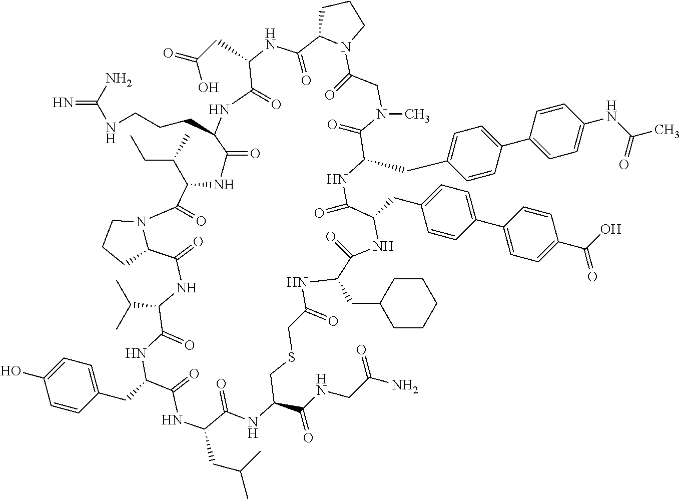 [SMILES: CC[C@H](C)[C@@H]1NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(C)=O)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2ccc(O)cc2)NC(=O)[C@H](C(C)C)NC(=O)[C@@H]2CCCN2C1=O]

Example 256 was prepared on a 50 umol scale. It was purified by Preparative LC/MS. The yield of the product was 20.9 mg, and its estimated purity by LCMS analysis was 96.5%. Analysis condition 1: Retention time=1.18 min. ESI-MS(+) m/z [M+2H]²⁺: 972.5.

### Preparation of Example 257

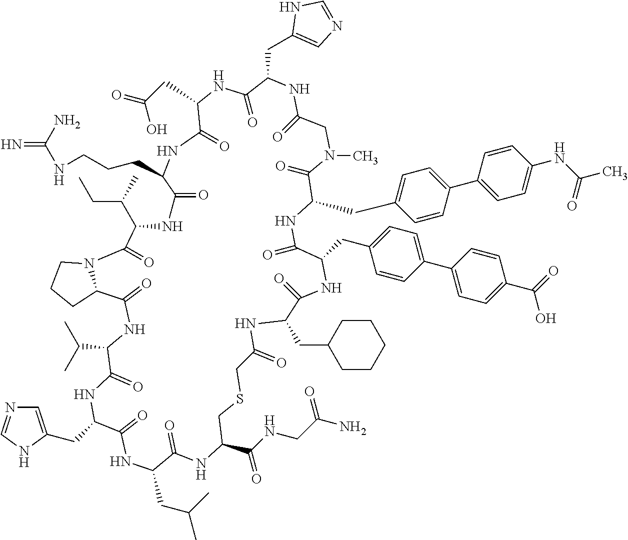 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(C)=O)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 257 was prepared on a 50 umol scale. It was purified by Preparative LC/MS. The yield of the product was 23.4 mg, and its estimated purity by LCMS analysis was 99.3%. Analysis condition 1: Retention time=1.23 min. ESI-MS(+) m/z [M+H]⁺: 1957.2.

### Preparation of Example 258

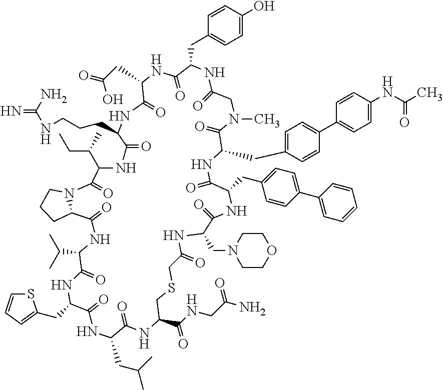 [SMILES: CC[C@H](C)C1NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccc(NC(C)=O)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2cccs2)NC(=O)[C@H](C(C)C)NC(=O)[C@@H]2CCCN2C1=O]

Example 258 was prepared on a 50 umol scale. It was purified by Preparative LC/MS. The yield of the product was 23.1 mg, and its estimated purity by LCMS analysis was 97.3%. Analysis condition 1: Retention time=1.52 min. ESI-MS(+) m/z [M+2H]²⁺: 980.1.

### Preparation of Example 259

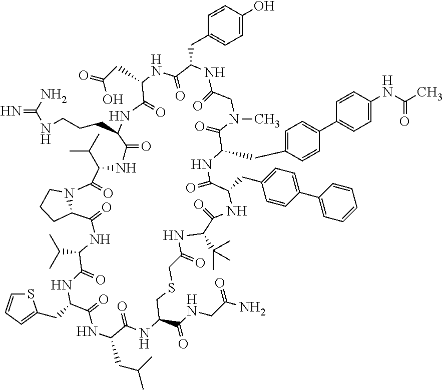 [SMILES: CC(=O)Nc1ccc(-c2ccc(C[C@@H]3NC(=O)[C@H](Cc4ccc(-c5ccccc5)cc4)NC(=O)[C@H](C(C)(C)C)NC(=O)CSC[C@@H](C(=O)NCC(N)=O)NC(=O)[C@H](CC(C)C)NC(=O)[C@H](Cc4cccs4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccc(O)cc4)NC(=O)CN(C)C3=O)cc2)cc1]

Example 259 was prepared on a 50 umol scale. It was purified by Preparative LC/MS. The yield of the product was 28 mg, and its estimated purity by LCMS analysis was 95.4%. Analysis condition 2: Retention time=1.51 min. ESI-MS(+) m/z [M+2H]²⁺: 951.

### Preparation of Example 260

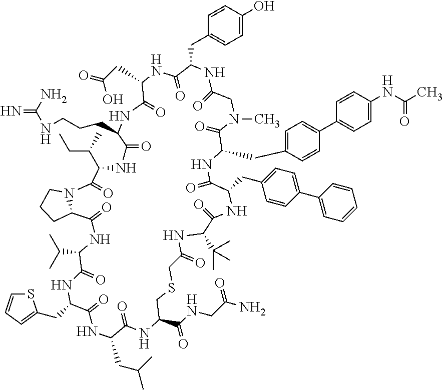 [SMILES: CC[C@H](C)[C@@H]1NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccc(NC(C)=O)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cccs2)NC(=O)[C@H](C(C)C)NC(=O)[C@@H]2CCCN2C1=O]

Example 260 was prepared on a 50 umol scale. It was purified by Preparative LC/MS. The yield of the product was 22.6 mg, and its estimated purity by LCMS analysis was 94.1%. Analysis condition 1: Retention time=1.47 min. ESI-MS(+) m/z [M+H]⁺: 1914.7.

### Preparation of Example 261

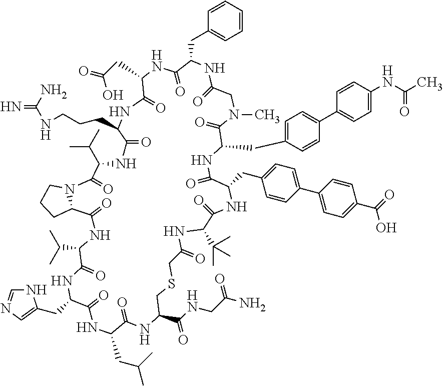 [SMILES: CC(=O)Nc1ccc(-c2ccc(C[C@@H]3NC(=O)[C@H](Cc4ccc(-c5ccc(C(=O)O)cc5)cc4)NC(=O)[C@H](C(C)(C)C)NC(=O)CSC[C@@H](C(=O)NCC(N)=O)NC(=O)[C@H](CC(C)C)NC(=O)[C@H](Cc4cnc[nH]4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccc4)NC(=O)CN(C)C3=O)cc2)cc1]

Example 261 was prepared on a 50 umol scale. It was purified by Preparative LC/MS. The yield of the product was 17.6 mg, and its estimated purity by LCMS analysis was 95%. Analysis condition 1: Retention time=1.2 min. ESI-MS(+) m/z [M+H]⁺: 1912.9.

### Preparation of Example 262

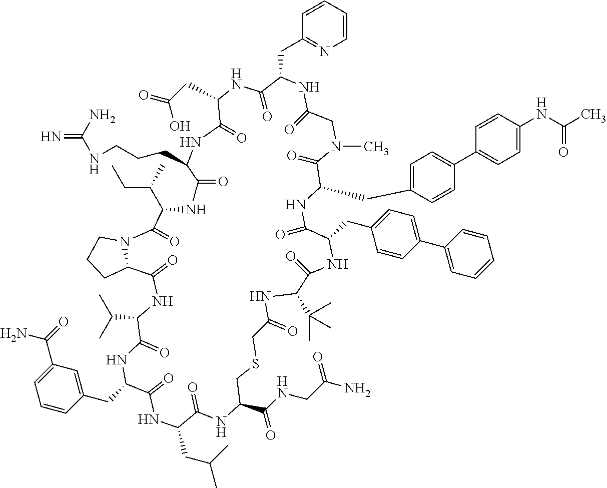 [SMILES: CC[C@H](C)[C@@H]1NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(C)=O)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cccc(C(N)=O)c2)NC(=O)[C@H](C(C)C)NC(=O)[C@@H]2CCCN2C1=O]

Example 262 was prepared on a 50 umol scale. It was purified by Preparative LC/MS. The yield of the product was 19.7 mg, and its estimated purity by LCMS analysis was 99.4%. Analysis condition 2: Retention time=1.43 min. ESI-MS(+) m/z [M+2H]²⁺: 969.4.

### Preparation of Example 263

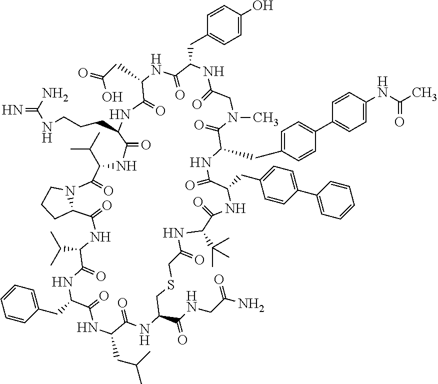 [SMILES: CC(=O)Nc1ccc(-c2ccc(C[C@@H]3NC(=O)[C@H](Cc4ccc(-c5ccccc5)cc4)NC(=O)[C@H](C(C)(C)C)NC(=O)CSC[C@@H](C(=O)NCC(N)=O)NC(=O)[C@H](CC(C)C)NC(=O)[C@H](Cc4ccccc4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccc(O)cc4)NC(=O)CN(C)C3=O)cc2)cc1]

Example 263 was prepared on a 50 umol scale. It was purified by Preparative LC/MS. The yield of the product was 17.9 mg, and its estimated purity by LCMS analysis was 100%. Analysis condition 1: Retention time=1.48 min. ESI-MS (+) m/z [M+H]⁺: 1894.9.

### Preparation of Example 264

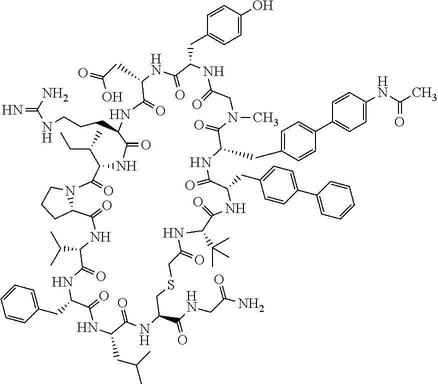 [SMILES: CC[C@H](C)[C@@H]1NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccc(NC(C)=O)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2ccccc2)NC(=O)[C@H](C(C)C)NC(=O)[C@@H]2CCCN2C1=O]

Example 264 was prepared on a 50 Preparative LC/MS. The yield of the product was 17.6 mg, and its estimated purity by LCMS analysis was 98.2%. Analysis condition 1: Retention time=1.49 min. ESI-MS (+) m/z [M+H]⁺: 1908.8.

### Preparation of Example 265

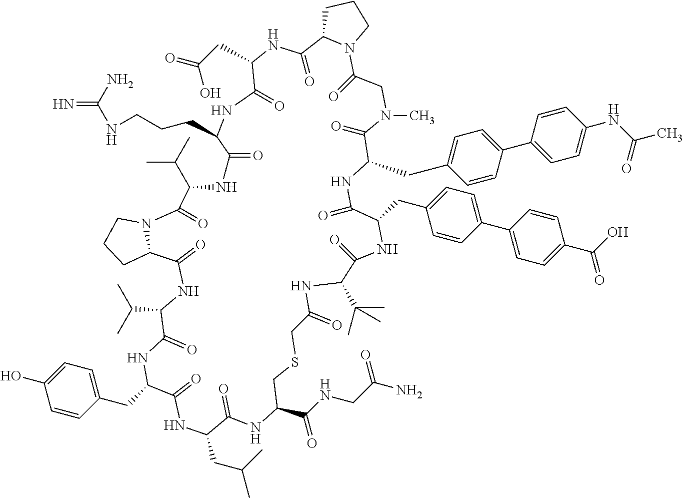 [SMILES: CC(=O)Nc1ccc(-c2ccc(C[C@@H]3NC(=O)[C@H](Cc4ccc(-c5ccc(C(=O)O)cc5)cc4)NC(=O)[C@H](C(C)(C)C)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 265 was prepared on a 50 umol scale. It was purified by Preparative LC/MS. The yield of the product was 17.4 mg, and its estimated purity by LCMS analysis was 100%. Analysis condition 2: Retention time=1.38 min. ESI-MS (+) m/z [M+H]⁺: 1889.5.

### Preparation of Example 266

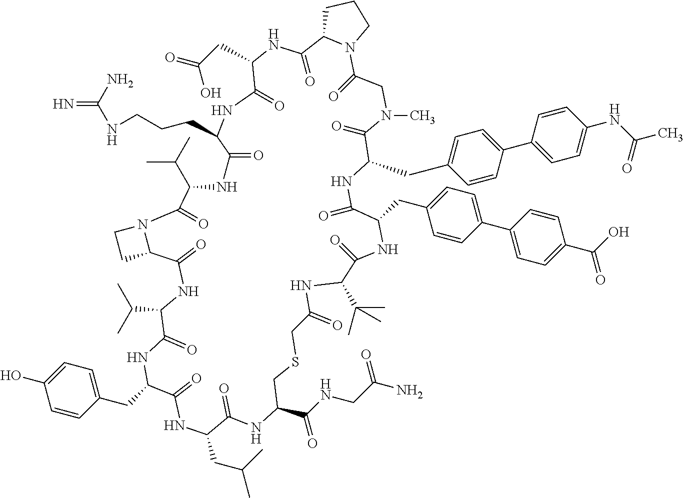 [SMILES: CC(=O)Nc1ccc(-c2ccc(C[C@@H]3NC(=O)[C@H](Cc4ccc(-c5ccc(C(=O)O)cc5)cc4)NC(=O)[C@H](C(C)(C)C)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 266 was prepared on a 50 Preparative LC/MS. The yield of the product was 15.9 mg, and its estimated purity by LCMS analysis was 98.2%. Analysis condition 1: Retention time=1.05 min. ESI-MS (+) m/z [M+2H]²⁺: 938.5.

### Preparation of Example 267

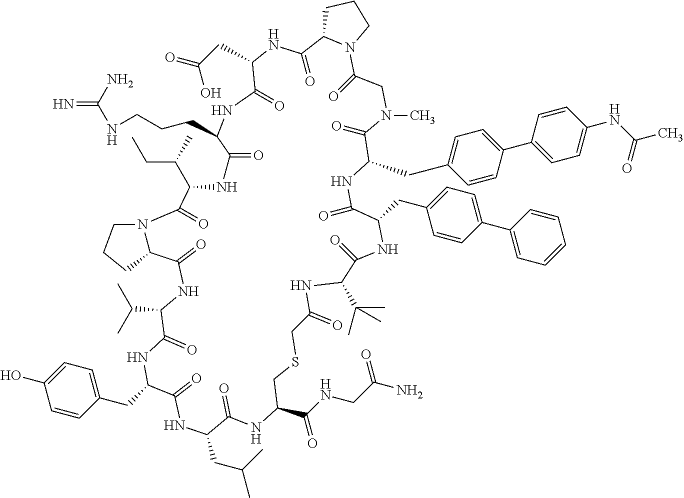 [SMILES: CC[C@H](C)[C@@H]1NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(C)=O)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2ccc(O)cc2)NC(=O)[C@H](C(C)C)NC(=O)[C@@H]2CCCN2C1=O]

Example 267 was prepared on a 50 umol scale. It was purified by Preparative LC/MS. The yield of the product was 27.2 mg, and its estimated purity by LCMS analysis was 100%. Analysis condition 1: Retention time=1.39 min. ESI-MS (+) m/z [M+H]⁺: 1858.8.

### Preparation of Example 268

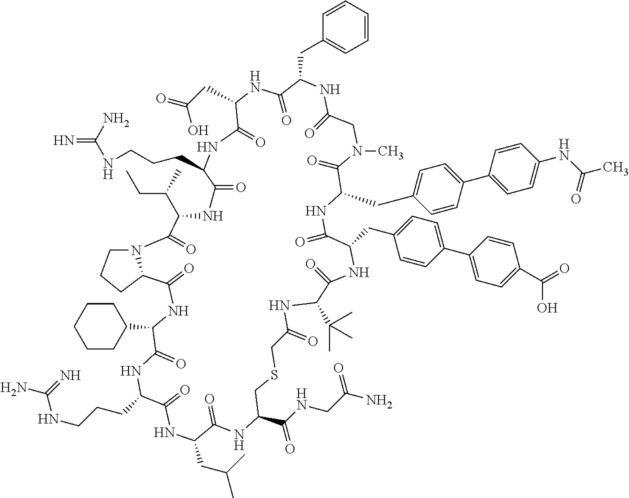 [SMILES: CC[C@H](C)[C@@H]1NC(=O)[C@@H](CCCNC(=N)N)NC(=O)[C@H](CC(=O)O)NC(=O)[C@H](Cc2ccccc2)NC(=O)CN(C)C(=O)[C@H](Cc2ccc(-c3ccc(NC(C)=O)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](CCCNC(=N)N)NC(=O)[C@H](C2CCCCC2)NC(=O)[C@@H]2CCCN2C1=O]

Example 268 was prepared on a 50 Preparative LC/MS. The yield of the product was 16.1 mg, and its estimated purity by LCMS analysis was 92.4%. Analysis condition 1: Retention time=1.32 min. ESI-MS (+) m/z [M+2H]²⁺: 993.9.

### Preparation of Example 269

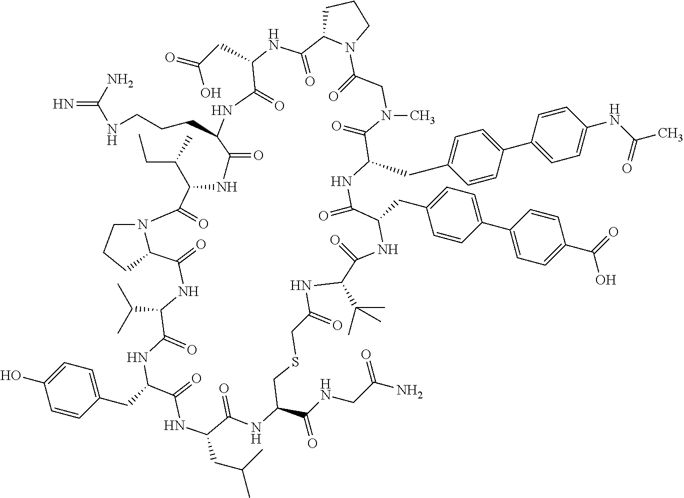 [SMILES: CC[C@H](C)[C@@H]1NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(C)=O)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2ccc(O)cc2)NC(=O)[C@H](C(C)C)NC(=O)[C@@H]2CCCN2C1=O]

Example 269 was prepared on a 50 umol scale. It was purified by Preparative LC/MS. The yield of the product was 17.9 mg, and its estimated purity by LCMS analysis was 92.4%. Analysis condition 1: Retention time=1.14 min. ESI-MS (+) m/z [M+H]⁺: 1902.9.

### Preparation of Example 270

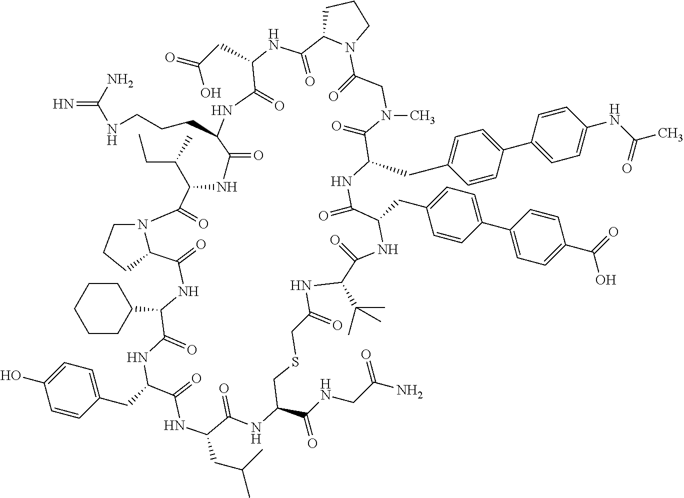 [SMILES: CC[C@H](C)[C@@H]1NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(C)=O)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2ccc(O)cc2)NC(=O)[C@H](C2CCCCC2)NC(=O)[C@@H]2CCCN2C1=O]

Example 270 was prepared on a 50 umol scale. It was purified by Preparative LC/MS. The yield of the product was 25.3 mg, and its estimated purity by LCMS analysis was 99.2%. Analysis condition 2: Retention time=1.45 min. ESI-MS (+) m/z [M+H]⁺: 1942.9.

### Preparation of Example 271

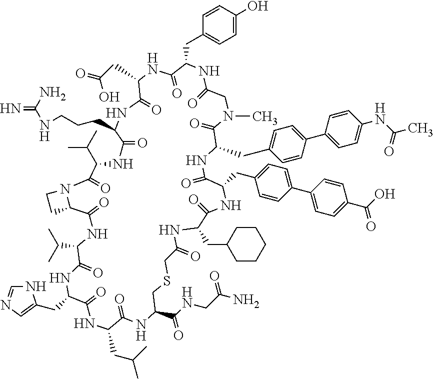 [SMILES: CC(=O)Nc1ccc(-c2ccc(C[C@@H]3NC(=O)[C@H](Cc4ccc(-c5ccc(C(=O)O)cc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4cnc[nH]4)NC(=O)[C@H](C(C)C)NC(=O)[C@@H]4CCN4C(=O)[C@H](C(C)C)NC(=O)[C@@H](CCCNC(=N)N)NC(=O)[C@H](CC(=O)O)NC(=O)[C@H](Cc4ccc(O)cc4)NC(=O)CN(C)C3=O)cc2)cc1]

Example 271 was prepared on a 50 umol scale. It was purified by Preparative LC/MS. The yield of the product was 21.1 mg, and its estimated purity by LCMS analysis was 100%. Analysis condition 1: Retention time=1.2 min. ESI-MS(+) m/z [M+2H]²⁺: 978.3.

### Preparation of Example 272

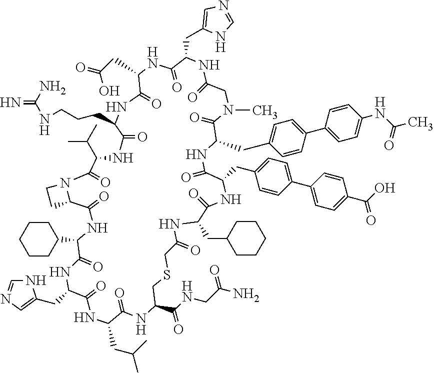 [SMILES: CC(=O)Nc1ccc(-c2ccc(C[C@@H]3NC(=O)[C@H](Cc4ccc(-c5ccc(C(=O)O)cc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4cnc[nH]4)NC(=O)[C@H](C4CCCCC4)NC(=O)[C@@H]4CCN4C(=O)[C@H](C(C)C)NC(=O)[C@@H](CCCNC(=N)N)NC(=O)[C@H](CC(=O)O)NC(=O)[C@H](Cc4cnc[nH]4)NC(=O)CN(C)C3=O)cc2)cc1]

Example 272 was prepared on a 50 umol scale. It was purified by Preparative LC/MS. The yield of the product was 11.1 mg, and its estimated purity by LCMS analysis was 100%. Analysis condition 1: Retention time=1.19 min. ESI-MS(+) m/z [M+2H]²⁺: 985.5.

### Preparation of Example 273

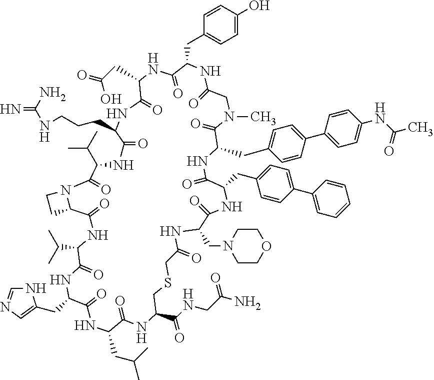 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cnc[nH]4)NC(=O)[C@H](C(C)C)NC(=O)[C@@H]4CCN4C(=O)[C@H](C(C)C)NC(=O)[C@@H](CCCNC(=N)N)NC(=O)[C@H](CC(=O)O)NC(=O)[C@H](Cc4ccc(O)cc4)NC(=O)CN(C)C3=O)cc2)cc1]

Example 273 was prepared on a 50 umol scale. It was purified by Preparative LC/MS. The yield of the product was 23.7 mg, and its estimated purity by LCMS analysis was 100%. Analysis condition 1: Retention time=1.43 min. ESI-MS(+) m/z [M+H]⁺: 1913.8.

### Preparation of Example 274

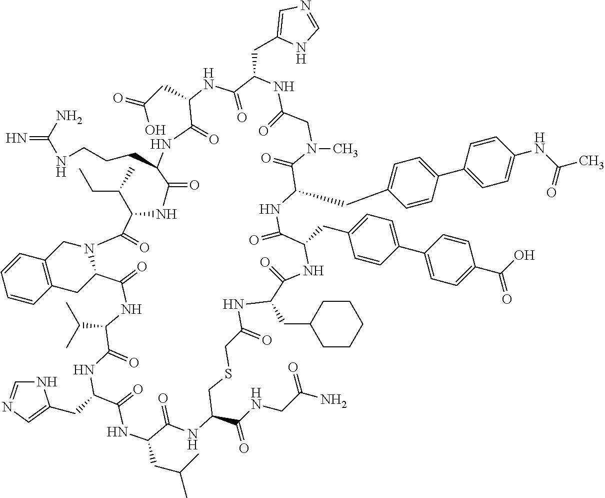 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(C)=O)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2Cc3ccccc3CN2C1=O]

Example 274 was prepared on a 50 umol scale. It was purified by Preparative LC/MS. The yield of the product was 15.1 mg, and its estimated purity by LCMS analysis was 93.9%. Analysis condition 2: Retention time=1.38 min. ESI-MS(+) m/z [M+2H]²⁺: 1010.4.

### Preparation of Example 275

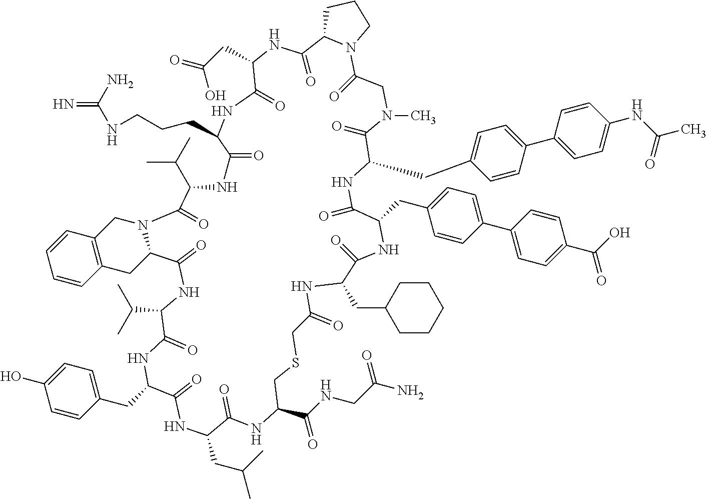 [SMILES: CC(=O)Nc1ccc(-c2ccc(C[C@@H]3NC(=O)[C@H](Cc4ccc(-c5ccc(C(=O)O)cc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4Cc5ccccc5CN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 275 was prepared on a 50 umol scale. It was purified by Preparative LC/MS. The yield of the product was 19.2 mg, and its estimated purity by LCMS analysis was 99.2%. Analysis condition 1: Retention time=1.3 min. ESI-MS(+) m/z [M+H]⁺: 1991.

### Preparation of Example 276

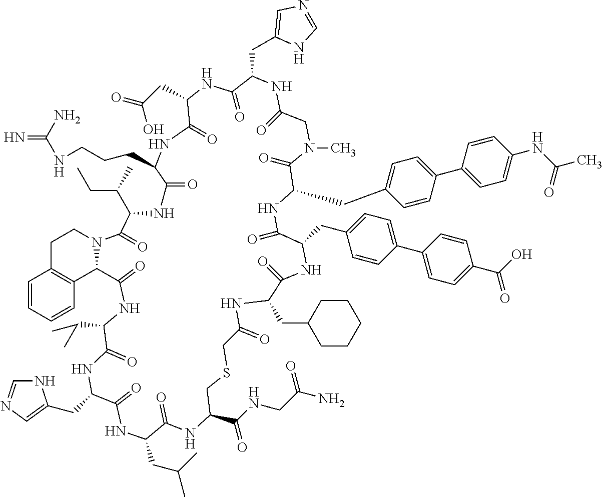 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(C)=O)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2c3ccccc3CCN2C1=O]

Example 276 was prepared on a 50 umol scale. It was purified by Preparative LC/MS. The yield of the product was 13.5 mg, and its estimated purity by LCMS analysis was 100%. Analysis condition 1: Retention time=1.32 min. ESI-MS(+) m/z [M+2H]²⁺: 1010.7.

### Preparation of Example 277

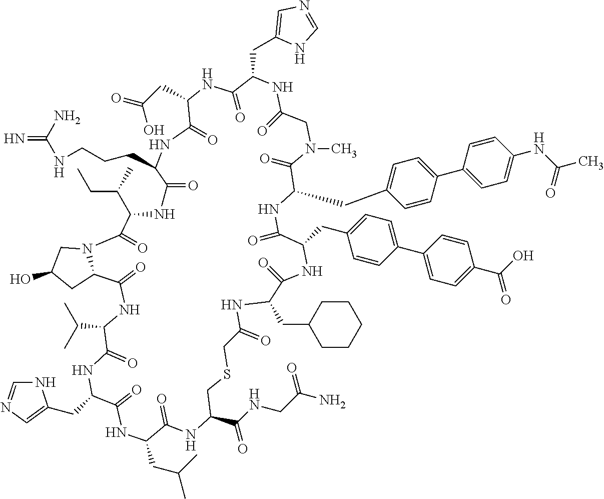 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(C)=O)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2C[C@@H](O)CN2C1=O]

Example 277 was prepared on a 50 umol scale. It was purified by Preparative LC/MS. The yield of the product was 19 mg, and its estimated purity by LCMS analysis was 100%. Analysis condition 1: Retention time=1.24 min. ESI-MS(+) m/z [M+H]⁺: 1972.9.

### Preparation of Example 278

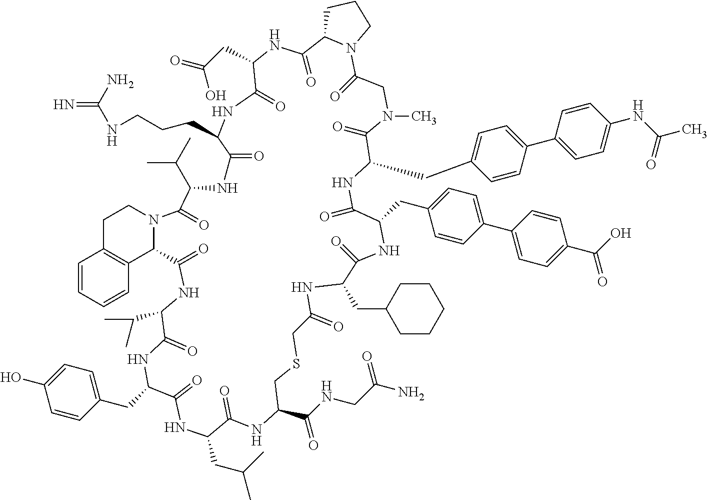 [SMILES: CC(=O)Nc1ccc(-c2ccc(C[C@@H]3NC(=O)[C@H](Cc4ccc(-c5ccc(C(=O)O)cc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4c5ccccc5CCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 278 was prepared on a 50 umol scale. It was purified by Preparative LC/MS. The yield of the product was 15.4 mg, and its estimated purity by LCMS analysis was 95.5%. Analysis condition 1: Retention time=1.3 min. ESI-MS(+) m/z [M+H]⁺: 1990.8.

### Preparation of Example 279

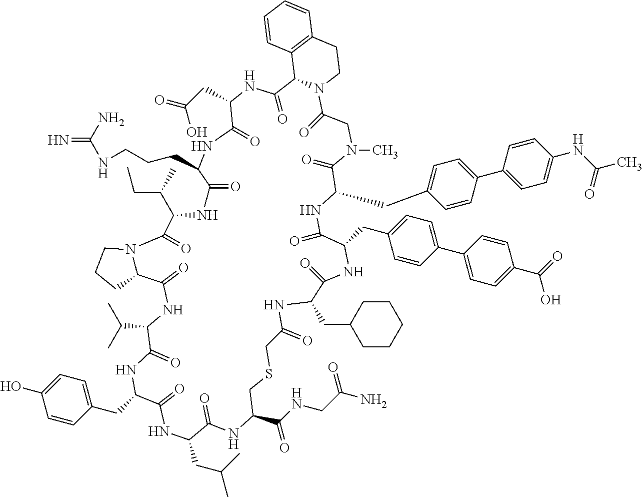 [SMILES: CC[C@H](C)[C@@H]1NC(=O)[C@@H](CCCNC(=N)N)NC(=O)[C@H](CC(=O)O)NC(=O)[C@@H]2c3ccccc3CCN2C(=O)CN(C)C(=O)[C@H](Cc2ccc(-c3ccc(NC(C)=O)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2ccc(O)cc2)NC(=O)[C@H](C(C)C)NC(=O)[C@@H]2CCCN2C1=O]

Example 279 was prepared on a 50 umol scale. It was purified by Preparative LC/MS. The yield of the product was 10.5 mg, and its estimated purity by LCMS analysis was 98.1%. Analysis condition 1: Retention time=1.32 min. ESI-MS(+) m/z [M+2H]²⁺: 1003.4.

### Preparation of Example 280

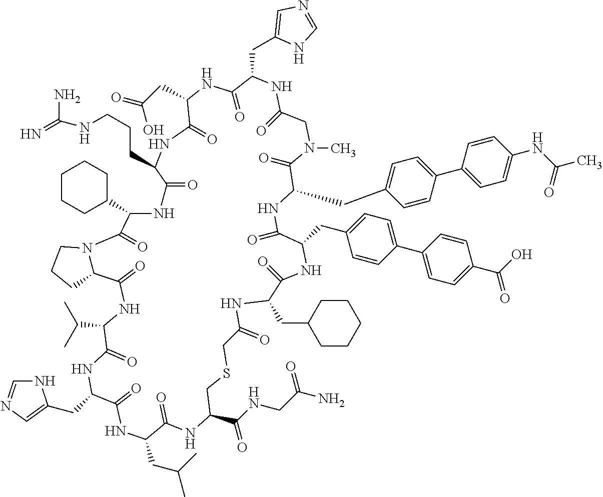 [SMILES: CC(=O)Nc1ccc(-c2ccc(C[C@@H]3NC(=O)[C@H](Cc4ccc(-c5ccc(C(=O)O)cc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4cnc[nH]4)NC(=O)[C@H](C(C)C)NC(=O)[C@@H]4CCCN4C(=O)[C@H](C4CCCCC4)NC(=O)[C@@H](CCCNC(=N)N)NC(=O)[C@H](CC(=O)O)NC(=O)[C@H](Cc4cnc[nH]4)NC(=O)CN(C)C3=O)cc2)cc1]

Example 280 was prepared on a 50 umol scale. It was purified by Preparative LC/MS. The yield of the product was 25.1 mg, and its estimated purity by LCMS analysis was 100%. Analysis condition 1: Retention time=1.24 min. ESI-MS(+) m/z [M+H]⁺: 1982.9.

### Preparation of Example 281

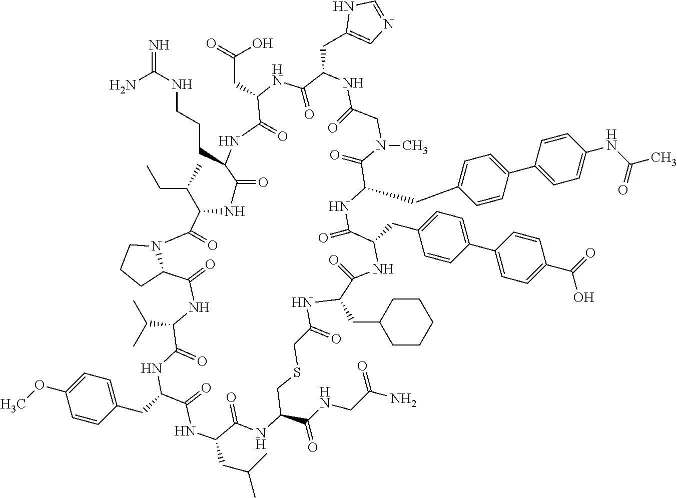 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(C)=O)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2ccc(OC)cc2)NC(=O)[C@H](C(C)C)NC(=O)[C@@H]2CCCN2C1=O]

Example 281 was prepared on a 50 Preparative LC/MS. The yield of the product was 19.2 mg, and its estimated purity by LCMS analysis was 96%. Analysis condition 1: Retention time=1.33 min. ESI-MS (+) m/z [M+H]⁺: 1996.9.

### Preparation of Example 282

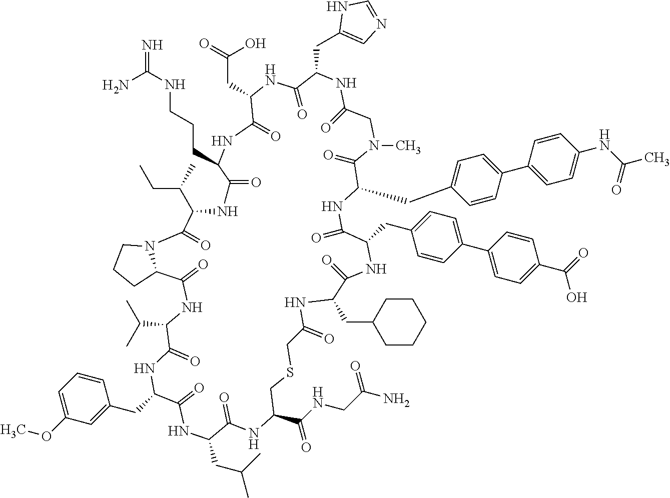 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(C)=O)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cccc(OC)c2)NC(=O)[C@H](C(C)C)NC(=O)[C@@H]2CCCN2C1=O]

Example 282 was prepared on a 50 umol scale. It was purified by Preparative LC/MS. The yield of the product was 19.3 mg, and its estimated purity by LCMS analysis was 94.7%. Analysis condition 2: Retention time=1.49 min. ESI-MS (+) m/z [M+H]⁺: 1996.8.

### Preparation of Example 283

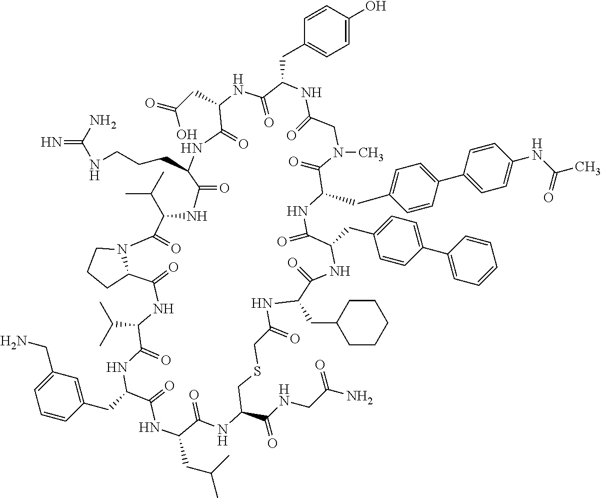 [SMILES: CC(=O)Nc1ccc(-c2ccc(C[C@@H]3NC(=O)[C@H](Cc4ccc(-c5ccccc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4cccc(CN)c4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccc(O)cc4)NC(=O)CN(C)C3=O)cc2)cc1]

Example 283 was prepared on a 50 Preparative LC/MS. The yield of the product was 16 mg, and its estimated purity by LCMS analysis was 91.6%. Analysis condition 2: Retention time=1.51 min. ESI-MS(+) m/z [M+H]⁺: 1963.6.

### Preparation of Example 284

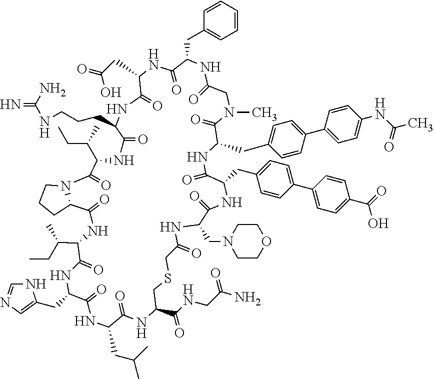 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccc2)NC(=O)CN(C)C(=O)[C@H](Cc2ccc(-c3ccc(NC(C)=O)cc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2cnc[nH]2)NC1=O]

Example 284 was prepared on a 50 umol scale. It was purified by Preparative LC/MS. The yield of the product was 20.3 mg, and its estimated purity by LCMS analysis was 100%. Analysis condition 1: Retention time=1.31 min. ESI-MS (+) m/z [M+H]⁺: 1983.8.

### Preparation of Example 285

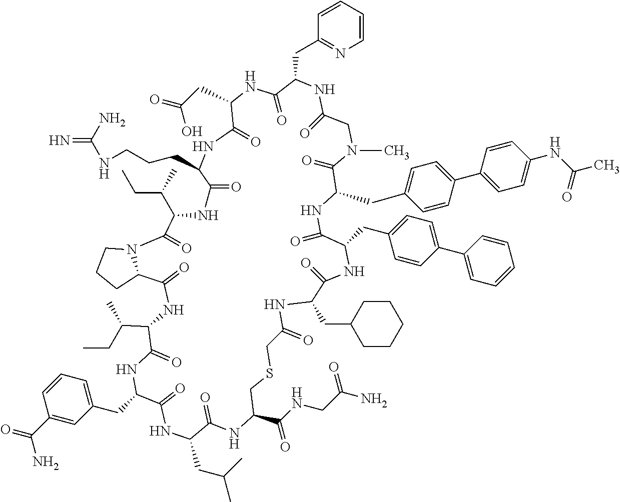 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(C)=O)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cccc(C(N)=O)c2)NC1=O]

Example 285 was prepared on a 50 Preparative LC/MS. The yield of the product was 7.6 mg, and its estimated purity by LCMS analysis was 99.3%. Analysis condition 1: Retention time=1.57 min. ESI-MS (+) m/z [M+H]⁺: 1990.9.

### Preparation of Example 286

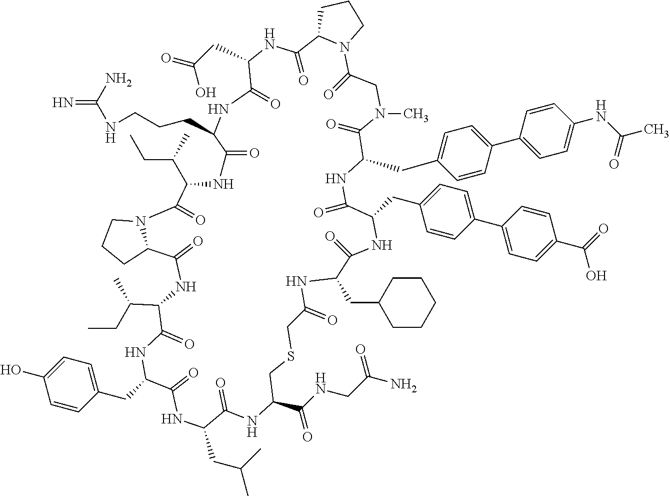 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(C)=O)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2ccc(O)cc2)NC1=O]

Example 286 was prepared on a 50 umol scale. It was purified by Preparative LC/MS. The yield of the product was 19.2 mg, and its estimated purity by LCMS analysis was 100%. Analysis condition 1: Retention time=1.26 min. ESI-MS (+) m/z [M+H]⁺: 1956.6.

### Preparation of Example 287

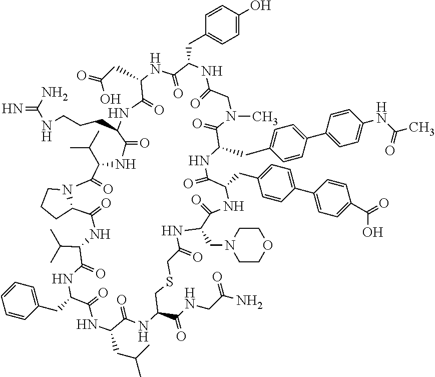 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccccc4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccc(O)cc4)NC(=O)CN(C)C3=O)cc2)cc1]

Example 287 was prepared on a 50 Preparative LC/MS. The yield of the product was 6.3 mg, and its estimated purity by LCMS analysis was 92.4%. Analysis condition 1: Retention time=1.28 min. ESI-MS (+) m/z [M+2H]²⁺: 991.9.

### Preparation of Example 288

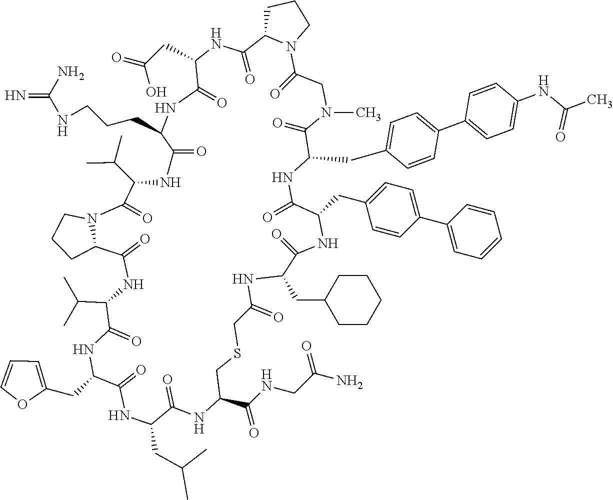 [SMILES: CC(=O)Nc1ccc(-c2ccc(C[C@@H]3NC(=O)[C@H](Cc4ccc(-c5ccccc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4ccco4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 288 was prepared on a 50 umol scale. It was purified by Preparative LC/MS. The yield of the product was 11.8 mg, and its estimated purity by LCMS analysis was 94.2%. Analysis condition 1: Retention time=1.42 min. ESI-MS (+) m/z [M+H]⁺: 1858.9.

### Preparation of Example 289

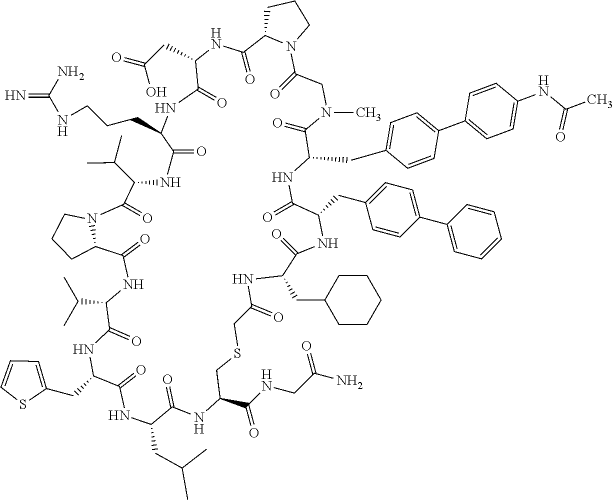 [SMILES: CC(=O)Nc1ccc(-c2ccc(C[C@@H]3NC(=O)[C@H](Cc4ccc(-c5ccccc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4cccs4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 289 was prepared on a 50 Preparative LC/MS. The yield of the product was 12.6 mg, and its estimated purity by LCMS analysis was 94.1%. Analysis condition 1: Retention time=1.42 min. ESI-MS (+) m/z [M+H]⁺: 1874.8.

### Preparation of Example 290

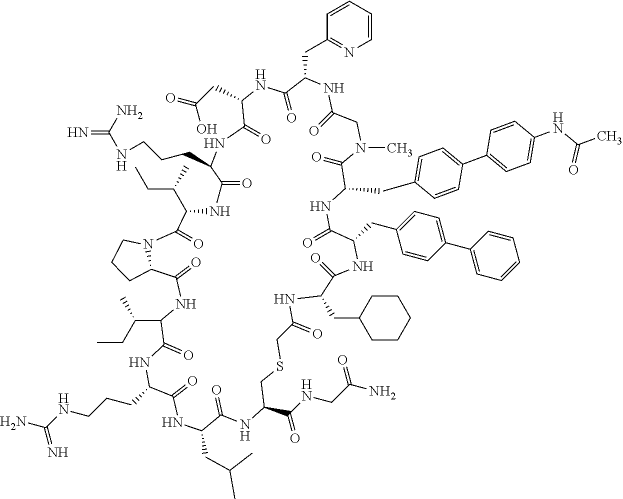 [SMILES: CC[C@H](C)C1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(C)=O)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](CCCNC(=N)N)NC1=O]

Example 290 was prepared on a 50 umol scale. It was purified by Preparative LC/MS. The yield of the product was 17.4 mg, and its estimated purity by LCMS analysis was 95.3%. Analysis condition 1: Retention time=1.58 min. ESI-MS (+) m/z [M+2H]²⁺: 979.4.

### Preparation of Example 291

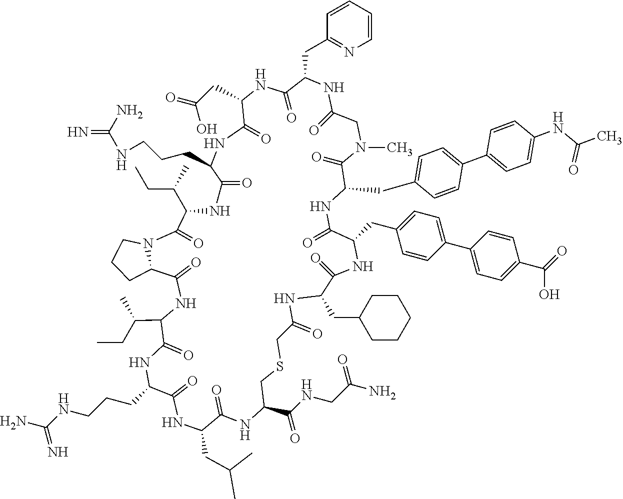 [SMILES: CC[C@H](C)C1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(C)=O)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](CCCNC(=N)N)NC1=O]

Example 291 was prepared on a 50 Preparative LC/MS. The yield of the product was 9.8 mg, and its estimated purity by LCMS analysis was 90.8%. Analysis condition 1: Retention time=1.37 min. ESI-MS (+) m/z [M+2H]²⁺: 1001.1.

### Preparation of Example 292

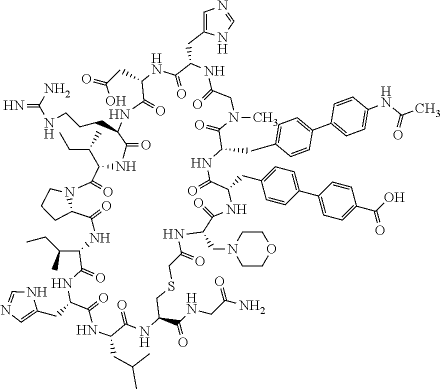 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(C)=O)cc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2cnc[nH]2)NC1=O]

Example 292 was prepared on a 50 umol scale. It was purified by Preparative LC/MS. The yield of the product was 20.7 mg, and its estimated purity by LCMS analysis was 100%. Analysis condition 1: Retention time=1.14 min. ESI-MS (+) m/z [M+H]⁺: 1973.8.

### Preparation of Example 293

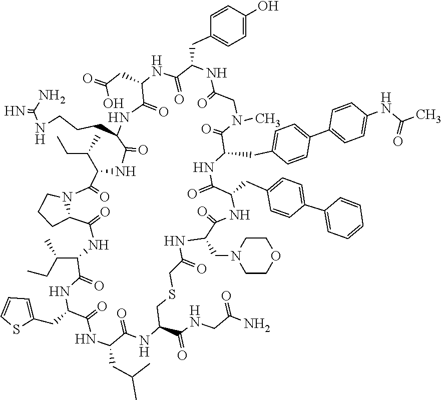 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccc(NC(C)=O)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2cccs2)NC1=O]

Example 293 was prepared on a 50 umol scale. It was purified by Preparative LC/MS. The yield of the product was 31.9 mg, and its estimated purity by LCMS analysis was 100%. Analysis condition 1: Retention time=1.55 min. ESI-MS(+) m/z [M+H]⁺: 1971.7.

### Preparation of Example 294

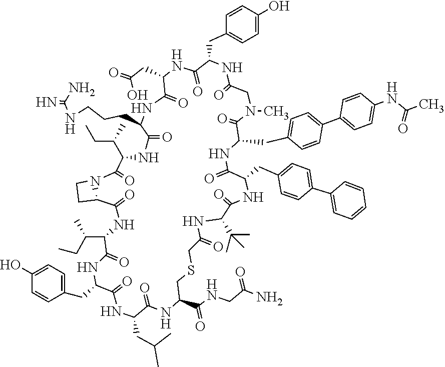 [SMILES: CC[C@H](C)[C@@H]1NC(=O)[C@@H]2CCN2C(=O)[C@H]([C@@H](C)CC)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccc(NC(C)=O)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2ccc(O)cc2)NC1=O]

Example 294 was prepared on a 50 umol scale. It was purified by Preparative LC/MS. The yield of the product was 19 mg, and its estimated purity by LCMS analysis was 92.7%. Analysis condition 2: Retention time=1.6 min. ESI-MS(+) m/z [M+H]⁺: 1924.6.

### Preparation of Example 295

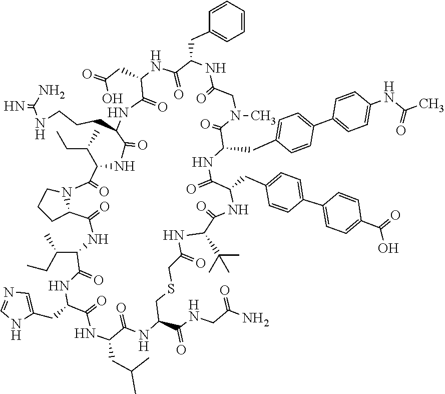 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccc2)NC(=O)CN(C)C(=O)[C@H](Cc2ccc(-c3ccc(NC(C)=O)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cnc[nH]2)NC1=O]

Example 295 was prepared on a 50 umol scale. It was purified by Preparative LC/MS. The yield of the product was 18 mg, and its estimated purity by LCMS analysis was 94.7%. Analysis condition 2: Retention time=1.49 min. ESI-MS(+) m/z [M+2H]²⁺: 971.4.

### Preparation of Example 296

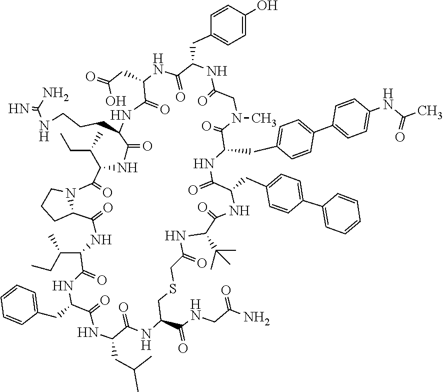 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccc(NC(C)=O)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2ccccc2)NC1=O]

Example 296 was prepared on a 50 umol scale. It was purified by Preparative LC/MS. The yield of the product was 31.3 mg, and its estimated purity by LCMS analysis was 91.2%. Analysis condition 1: Retention time=1.56 min. ESI-MS(+) m/z [M+H]⁺: 1922.7.

### Preparation of Example 297

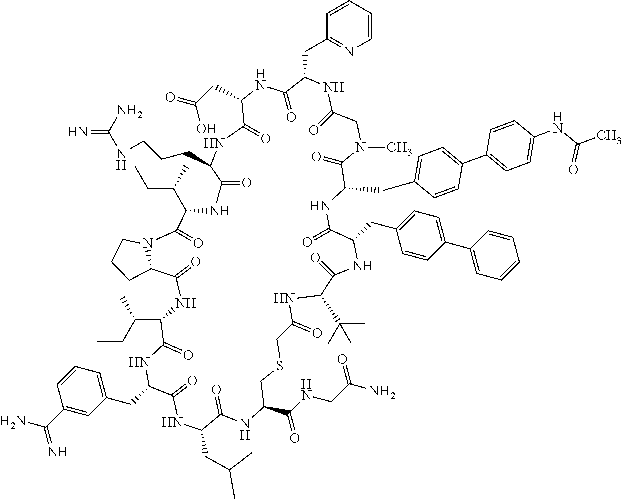 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(C)=O)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cccc(C(=N)N)c2)NC1=O]

Example 297 was prepared on a 50 Preparative LC/MS. The yield of the product was 26.4 mg, and its estimated purity by LCMS analysis was 93.1%. Analysis condition 1: Retention time=1.52 min. ESI-MS (+) m/z [M+H]⁺: 1950.6.

### Preparation of Example 298

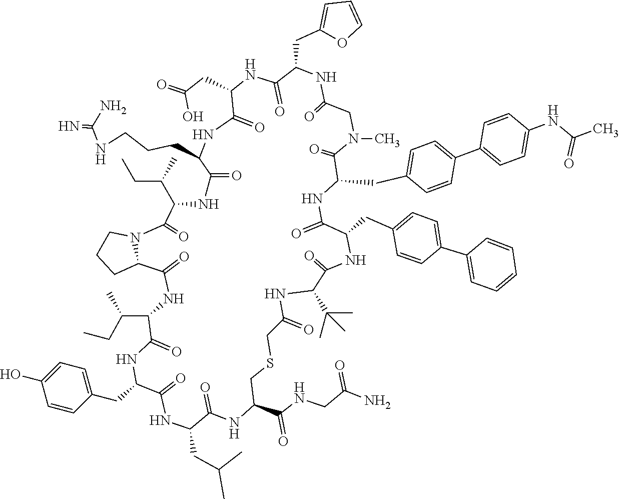 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccco2)NC(=O)CN(C)C(=O)[C@H](Cc2ccc(-c3ccc(NC(C)=O)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2ccc(O)cc2)NC1=O]

Example 298 was prepared on a 50 umol scale. It was purified by Preparative LC/MS. The yield of the product was 29.2 mg, and its estimated purity by LCMS analysis was 96.6%. Analysis condition 1: Retention time=1.51 min. ESI-MS(+) m/z [M+H]⁺: 1912.6.

### Preparation of Example 299

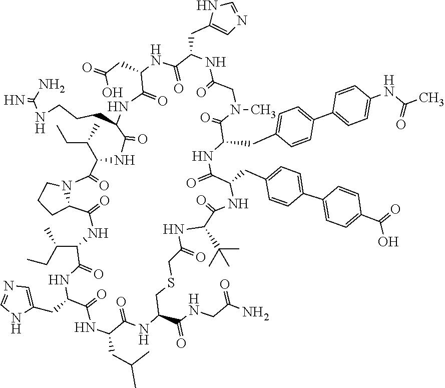 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(C)=O)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cnc[nH]2)NC1=O]

Example 299 was prepared on a 50 umol scale. It was purified by Preparative LC/MS. The yield of the product was 23.1 mg, and its estimated purity by LCMS analysis was 95.4%. Analysis condition 2: Retention time=1.29 min. ESI-MS(+) m/z [M+H]⁺: 1886.7.

### Preparation of Example 300

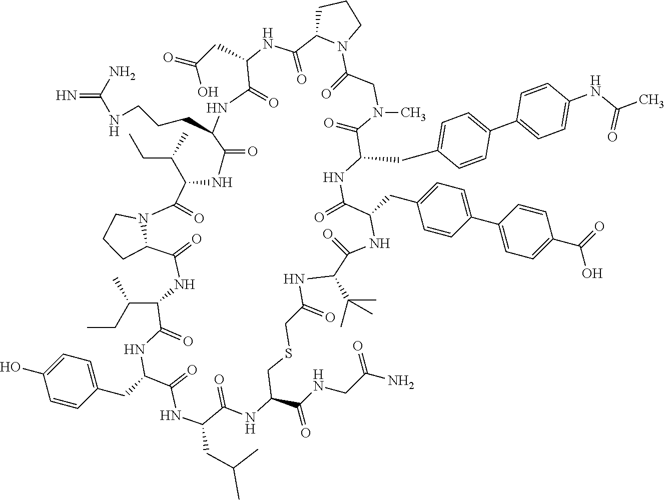 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(C)=O)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2ccc(O)cc2)NC1=O]

Example 300 was prepared on a 50 umol scale. It was purified by Preparative LC/MS. The yield of the product was 24.4 mg, and its estimated purity by LCMS analysis was 96.3%. Analysis condition 2: Retention time=1.45 min. ESI-MS(+) m/z [M+H]⁺: 1917.5.

### Preparation of Example 301

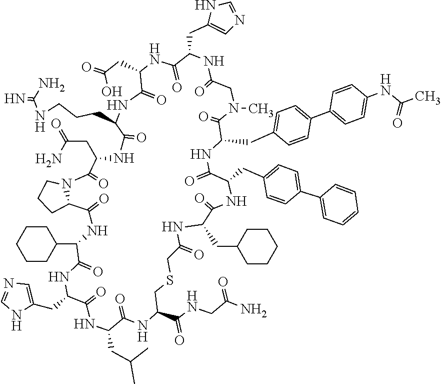 [SMILES: CC(=O)Nc1ccc(-c2ccc(C[C@@H]3NC(=O)[C@H](Cc4ccc(-c5ccccc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4cnc[nH]4)NC(=O)[C@H](C4CCCCC4)NC(=O)[C@@H]4CCCN4C(=O)[C@H](CC(N)=O)NC(=O)[C@@H](CCCNC(=N)N)NC(=O)[C@H](CC(=O)O)NC(=O)[C@H](Cc4cnc[nH]4)NC(=O)CN(C)C3=O)cc2)cc1]

Example 301 was prepared on a 50 umol scale. It was purified by Preparative LC/MS. The yield of the product was 28.4 mg, and its estimated purity by LCMS analysis was 95.1%. Analysis condition 1: Retention time=1.42 min. ESI-MS(+) m/z [M+H]⁺: 1954.7.

### Preparation of Example 302

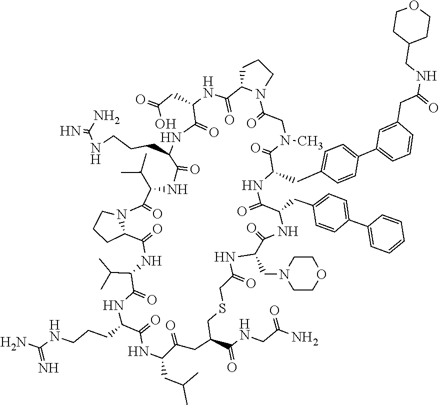 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3cccc(CC(=O)NCC4CCOCC4)c3)cc2)NC(=O)[C@H](Cc2ccc(-c3ccccc3)cc2)NC(=O)[C@H](CN2CCOCC2)NC(=O)CSC[C@@H](C(=O)NCC(N)=O)CC1=O]

Example 302 was prepared on a 10 umol scale. It was purified by Preparative LC/MS. The yield of the product was 4.4 mg, and its estimated purity by LCMS analysis was 91.1%. Analysis condition 1: Retention time=1.39 min. ESI-MS (+) m/z [M+2H]²⁺: 990.5.

### Preparation of Example 303

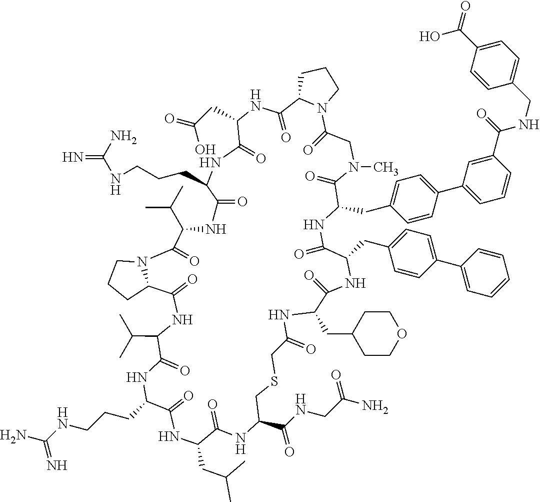 [SMILES: CC(C)C[C@@H]1NC(=O)[C@H](CCCNC(=N)N)NC(=O)C(C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3cccc(C(=O)NCc4ccc(C(=O)O)cc4)c3)cc2)NC(=O)[C@H](Cc2ccc(-c3ccccc3)cc2)NC(=O)[C@H](CC2CCOCC2)NC(=O)CSC[C@@H](C(=O)NCC(N)=O)NC1=O]

Example 303 was prepared on a 6.1 umol scale. It was purified by Preparative LC/MS. The yield of the product was 8 mg, and its estimated purity by LCMS analysis was 94%. Analysis condition 1: Retention time=1.28 min. ESI-MS(+) m/z [M+2H]²⁺: 1001.4.

### Preparation of Example 304

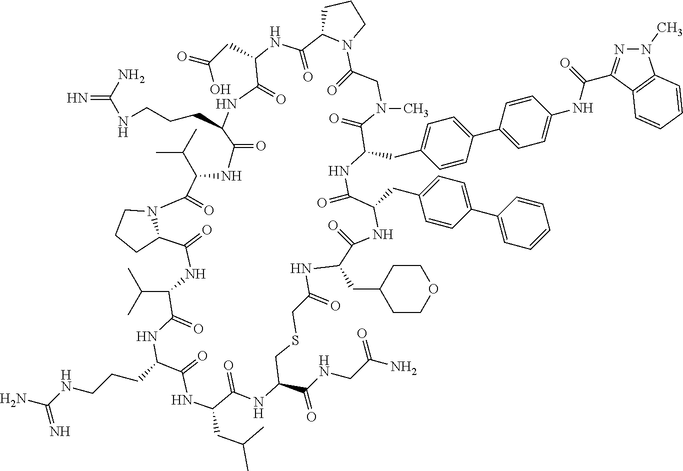 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](CC2CCOCC2)NC(=O)CSC[C@@H](C(=O)NCC(N)=O)NC1=O]

Example 304 was prepared on a 6.1 umol scale. It was purified by Preparative LC/MS. The yield of the product was 2.7 mg, and its estimated purity by LCMS analysis was 100%. Analysis condition 2: Retention time=1.52 min. ESI-MS (+) m/z [M+2H]²⁺: 999.4.

### Preparation of Example 305

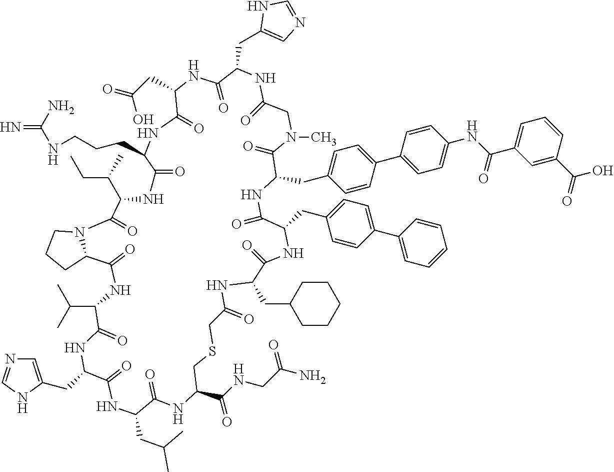 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(C(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 305 was prepared on a 100 Preparative LC/MS. The yield of the product was 23 mg, and its estimated purity by LCMS analysis was 95.7%. Analysis condition 1: Retention time=1.32 min. ESI-MS(+) m/z [M+2H]²⁺: 1010.5.

### Preparation of Example 306

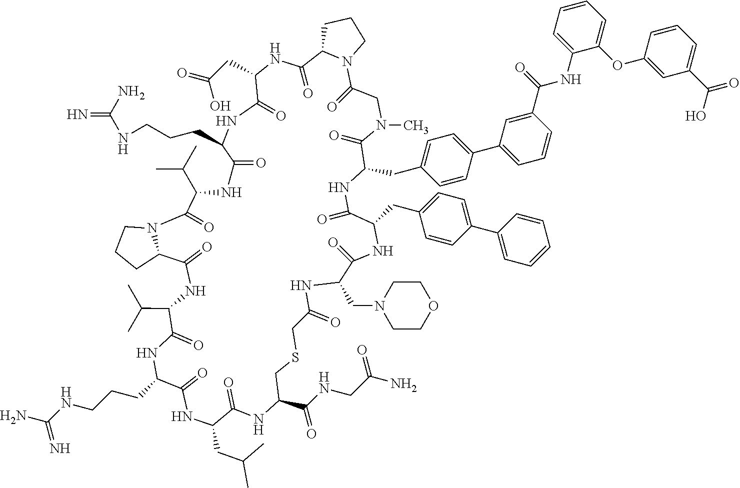 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3cccc(C(=O)Nc4ccccc4Oc4cccc(C(=O)O)c4)c3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 306 was prepared on a 6.1 umol scale. It was purified by Preparative LC/MS. The yield of the product was 3.6 mg, and its estimated purity by LCMS analysis was 94.1%. Analysis condition 1: Retention time=1.37 min. ESI-MS (+) m/z [M+2H]²⁺: 1040.5.

### Preparation of Example 307

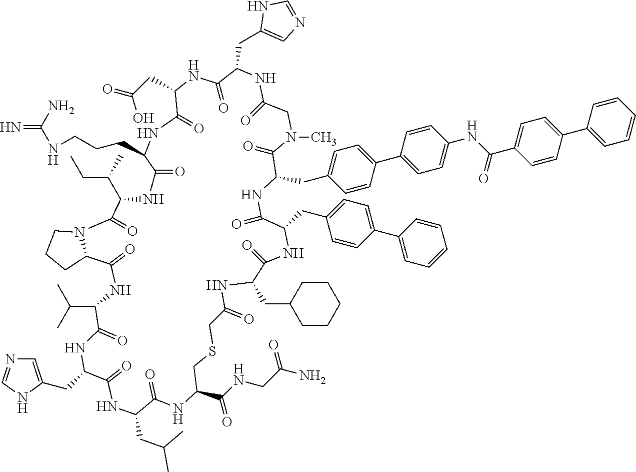 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccc(-c5ccccc5)cc4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 307 was prepared on a 25 Preparative LC/MS. The yield of the product was 9.5 mg, and its estimated purity by LCMS analysis was 90.5%. Analysis condition 2: Retention time=1.66 min. ESI-MS (+) m/z [M+2H]²⁺: 1026.5.

### Preparation of Example 308

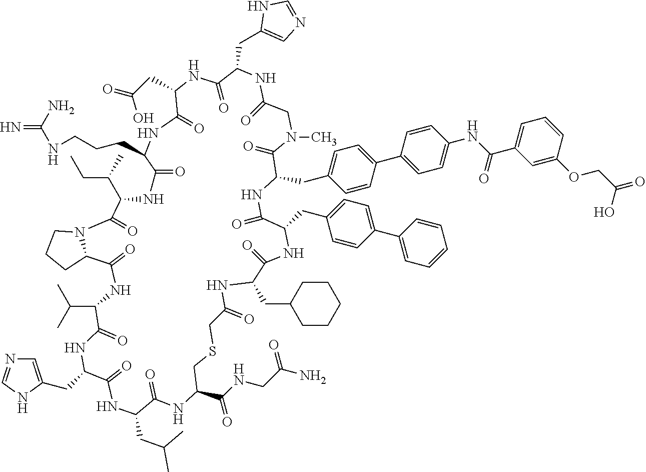 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 308 was prepared on a 50 umol scale. It was purified by Preparative LC/MS. The yield of the product was 17.2 mg, and its estimated purity by LCMS analysis was 90.6%. Analysis condition 2: Retention time=1.36 min. ESI-MS (+) m/z [M+2H]²⁺: 1025.6.

### Preparation of Example 309

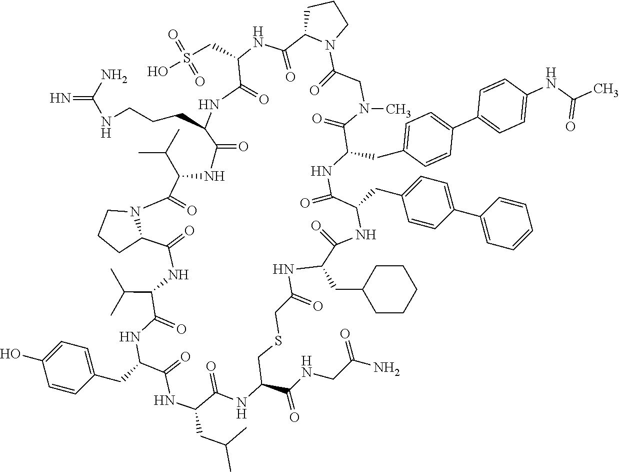 [SMILES: CC(=O)Nc1ccc(-c2ccc(C[C@@H]3NC(=O)[C@H](Cc4ccc(-c5ccccc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](C(C)C)NC(=O)[C@@H](CCCNC(=N)N)NC(=O)[C@H](CS(=O)(=O)O)NC(=O)[C@@H]4CCCN4C(=O)CN(C)C3=O)cc2)cc1]

Example 309 was prepared on a 50 Preparative LC/MS. The yield of the product was 25.8 mg, and its estimated purity by LCMS analysis was 97.4%. Analysis condition 1: Retention time=1.41 min. ESI-MS (+) m/z [M+H]⁺: 1921.6.

### Preparation of Example 310

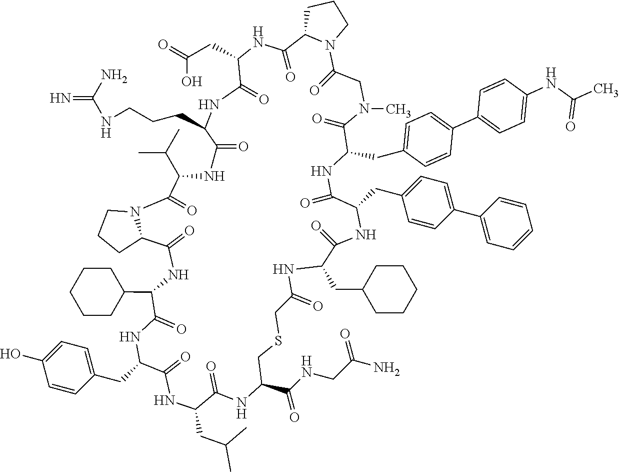 [SMILES: CC(=O)Nc1ccc(-c2ccc(C[C@@H]3NC(=O)[C@H](Cc4ccc(-c5ccccc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4ccc(O)cc4)NC(=O)[C@H](C4CCCCC4)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 310 was prepared on a 50 umol scale. It was purified by Preparative LC/MS. The yield of the product was 30 mg, and its estimated purity by LCMS analysis was 95.5%. Analysis condition 1: Retention time=1.44 min. ESI-MS(+) m/z [M+H]⁺: 1924.7.

### Preparation of Example 311

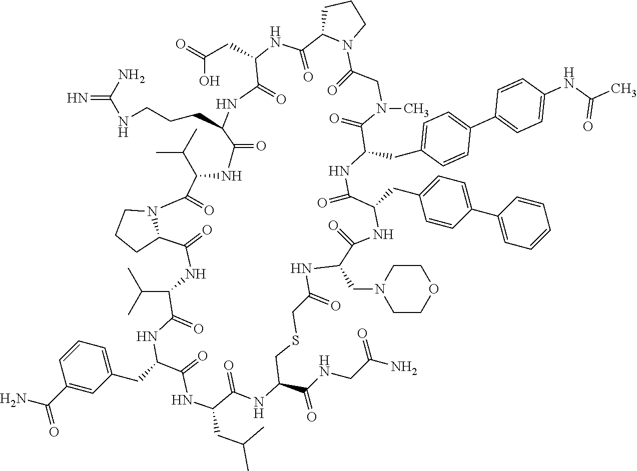 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cccc(C(N)=O)c4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 311 was prepared on a 50 Preparative LC/MS. The yield of the product was 30.3 mg, and its estimated purity by LCMS analysis was 100%. Analysis condition 1: Retention time=1.3 min. ESI-MS (+) m/z [M+H]⁺: 1914.7.

### Preparation of Example 312

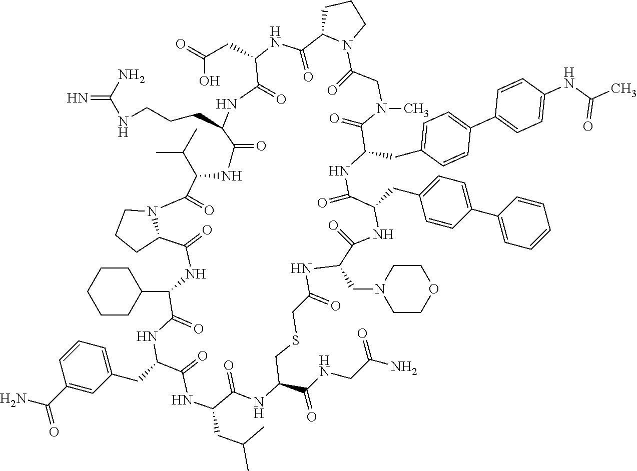 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cccc(C(N)=O)c4)NC(=O)[C@H](C4CCCCC4)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 312 was prepared on a 50 umol scale. It was purified by Preparative LC/MS. The yield of the product was 34.9 mg, and its estimated purity by LCMS analysis was 100%. Analysis condition 1: Retention time=1.35 min. ESI-MS (+) m/z [M+2H]²⁺: 978.4.

### Preparation of Example 313

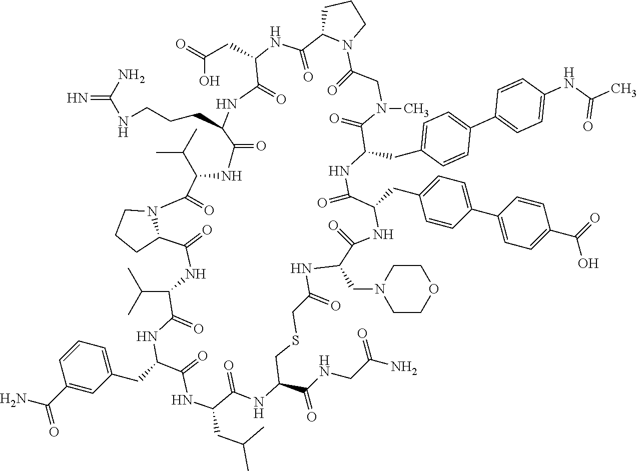 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cccc(C(N)=O)c4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 313 was prepared on a 50 Preparative LC/MS. The yield of the product was 17.9 mg, and its estimated purity by LCMS analysis was 100%. Analysis condition 1: Retention time=1.05 min. ESI-MS (+) m/z [M+H]⁺: 1958.5.

### Preparation of Example 314

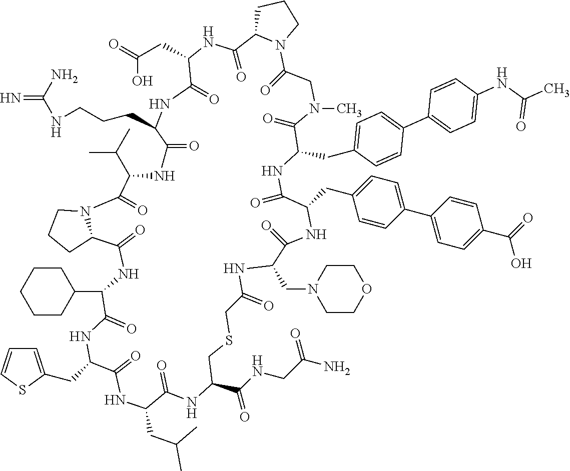 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cccs4)NC(=O)[C@H](C4CCCCC4)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 314 was prepared on a 50 umol scale. It was purified by Preparative LC/MS. The yield of the product was 25 mg, and its estimated purity by LCMS analysis was 95.6%. Analysis condition 1: Retention time=1.2 min. ESI-MS(+) m/z [M+H]⁺: 1961.7.

### Preparation of Example 315

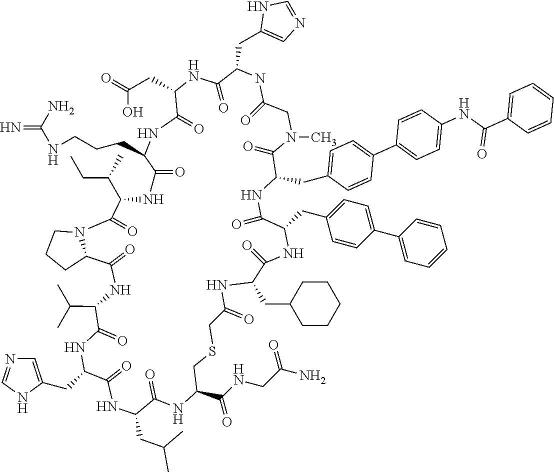 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccccc4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 315 was prepared on a 100 Preparative LC/MS. The yield of the product was 55.3 mg, and its estimated purity by LCMS analysis was 100%. Analysis condition 1: Retention time=1.7 min. ESI-MS (+) m/z [M+H]⁺: 1975.2.

### Preparation of Example 316

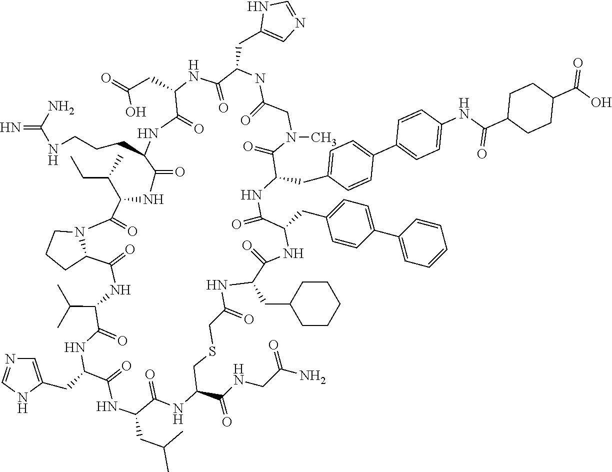 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)C4CCC(C(=O)O)CC4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 316 was prepared on a 50 umol scale. It was purified by Preparative LC/MS. The yield of the product was 15.2 mg, and its estimated purity by LCMS analysis was 100%. Analysis condition 1: Retention time=1.35 min. ESI-MS (+) m/z [M+2H] 2+: 1013.3.

### Preparation of Example 317

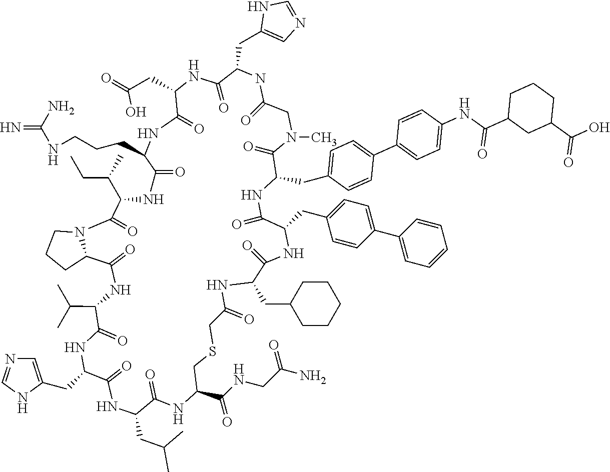 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)C4CCCC(C(=O)O)C4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 317 was prepared on a 50 Preparative LC/MS. The yield of the product was 18.6 mg, and its estimated purity by LCMS analysis was 100%. Analysis condition 1: Retention time=1.37 min. ESI-MS (+) m/z [M+2H]²⁺: 1013.3.

### Preparation of Example 318

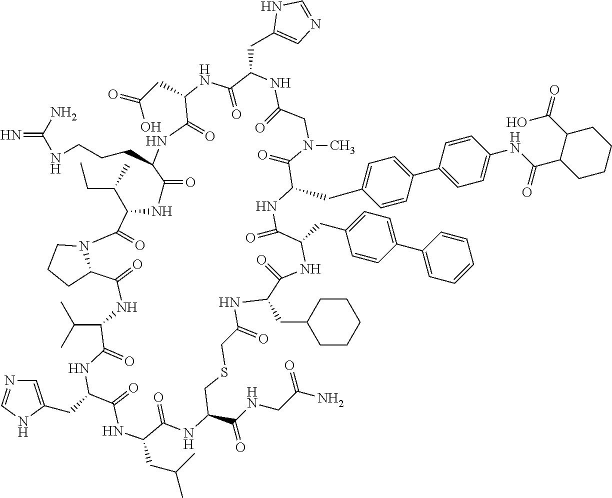 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)C4CCCCC4C(=O)O)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 318 was prepared on a 50 umol scale. It was purified by Preparative LC/MS. The yield of the product was 3.7 mg, and its estimated purity by LCMS analysis was 91%. Analysis condition 2: Retention time=1.51 min. ESI-MS (+) m/z [M+2H]²⁺: 1013.2.

### Preparation of Example 319

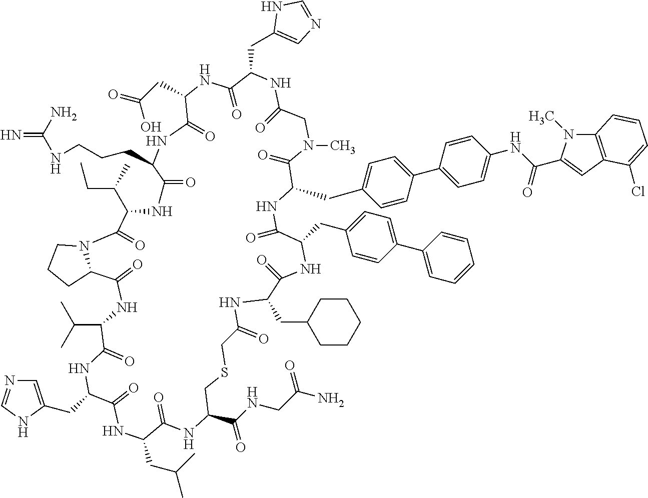 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cc5c(Cl)cccc5n4C)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 319 was prepared on a 50 Preparative LC/MS. The yield of the product was 21 mg, and its estimated purity by LCMS analysis was 100%. Analysis condition 1: Retention time=1.84 min. ESI-MS(+) m/z [M+2H]²⁺: 1031.8.

### Preparation of Example 320

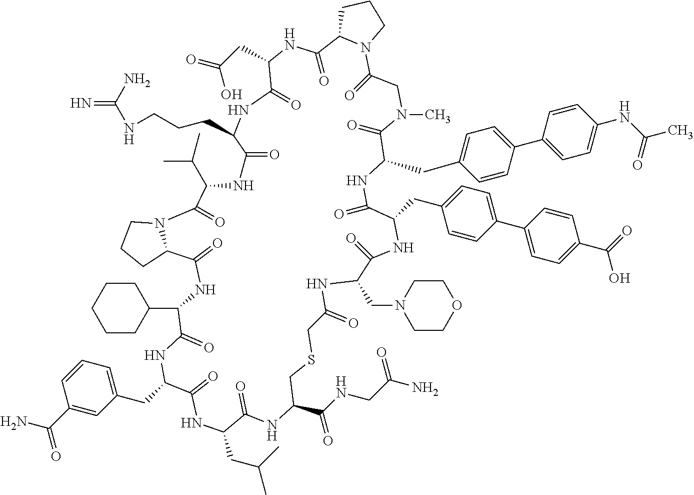 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cccc(C(N)=O)c4)NC(=O)[C@H](C4CCCCC4)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 320 was prepared on a 50 umol scale. It was purified by Preparative LC/MS. The yield of the product was 23.9 mg, and its estimated purity by LCMS analysis was 100%. Analysis condition 1: Retention time=1.2 min. ESI-MS (+) m/z [M+H]⁺: 1998.7.

### Preparation of Example 321

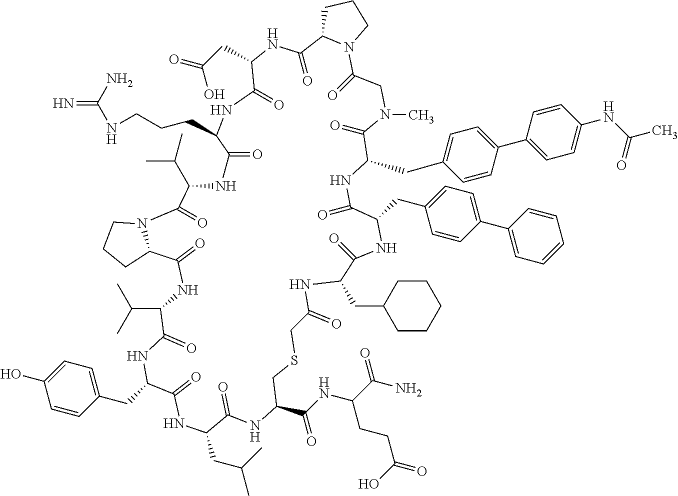 [SMILES: CC(=O)Nc1ccc(-c2ccc(C[C@@H]3NC(=O)[C@H](Cc4ccc(-c5ccccc5)cc4)NC(=O)[C@H](CC4CCCCC4)NC(=O)CSC[C@@H](C(=O)NC(CCC(=O)O)C(N)=O)NC(=O)[C@H](CC(C)C)NC(=O)[C@H](Cc4ccc(O)cc4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 321 was prepared on a 50 Preparative LC/MS. The yield of the product was 19.1 mg, and its estimated purity by LCMS analysis was 100%. Analysis condition 2: Retention time=1.53 min. ESI-MS (+) m/z [M+H]⁺: 1956.8.

### Preparation of Example 322

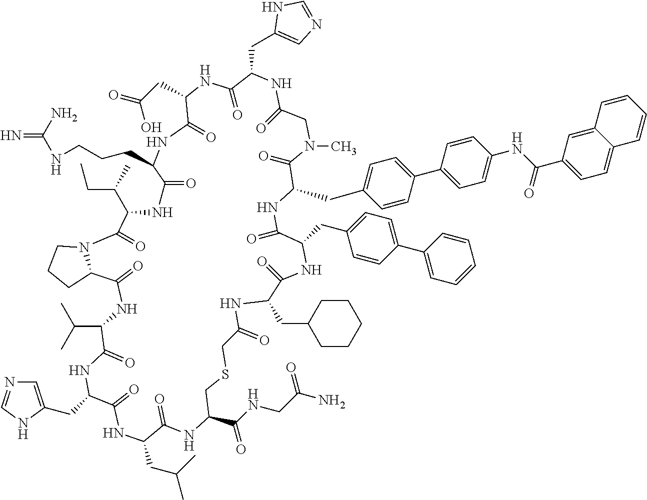 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccc5ccccc5c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 322 was prepared on a 50 umol scale. It was purified by Preparative LC/MS. The yield of the product was 10.3 mg, and its estimated purity by LCMS analysis was 100%. Analysis condition 1: Retention time=1.85 min. ESI-MS (+) m/z [M+2H]²⁺: 1013.6.

### Preparation of Example 323

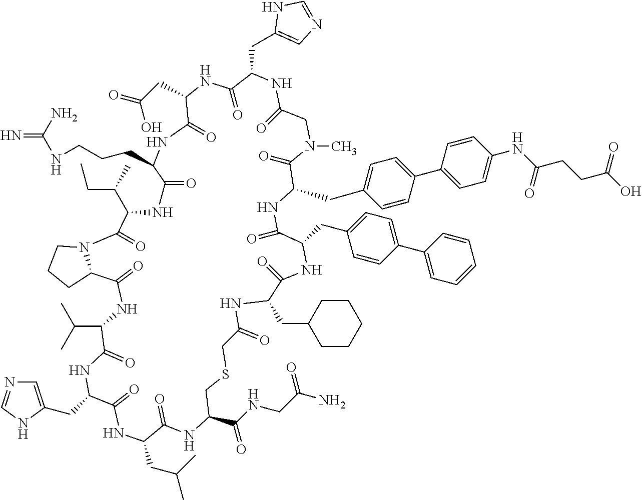 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)CCC(=O)O)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 323 was prepared on a 50 Preparative LC/MS. The yield of the product was 8.6 mg, and its estimated purity by LCMS analysis was 100%. Analysis condition 2: Retention time=1.36 min. ESI-MS (+) m/z [M+H]⁺: 1970.8.

### Preparation of Example 324

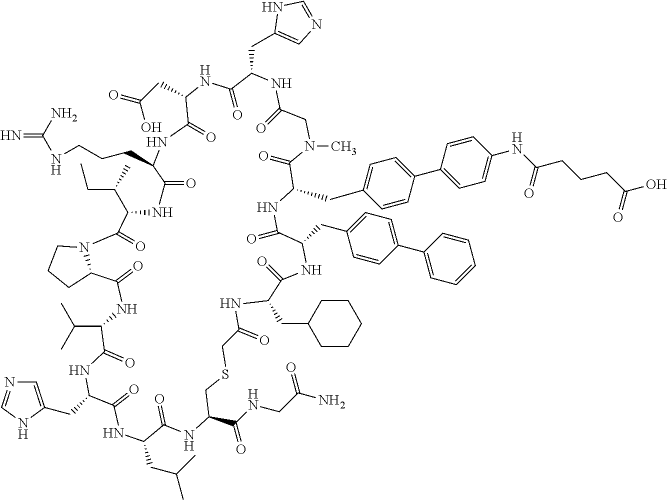 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)CCCC(=O)O)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 324 was prepared on a 50 umol scale. It was purified by Preparative LC/MS. The yield of the product was 15.2 mg, and its estimated purity by LCMS analysis was 100%. Analysis condition 1: Retention time=1.43 min. ESI-MS (+) m/z [M+H]⁺: 1984.7.

### Preparation of Example 325

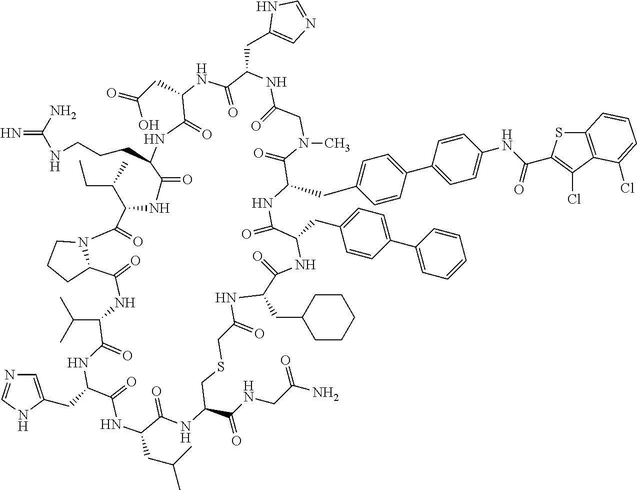 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4sc5cccc(Cl)c5c4Cl)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 325 was prepared on a 40 Preparative LC/MS. The yield of the product was 10.4 mg, and its estimated purity by LCMS analysis was 91%. Analysis condition 2: Retention time=1.81 min. ESI-MS (+) m/z [M+2H]²⁺: 1050.9.

### Preparation of Example 326

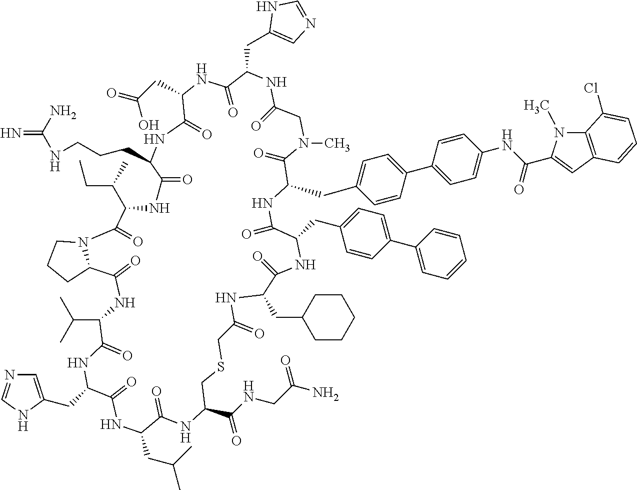 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cc5cccc(Cl)c5n4C)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 326 was prepared on a 40 umol scale. It was purified by Preparative LC/MS. The yield of the product was 12.9 mg, and its estimated purity by LCMS analysis was 93.8%. Analysis condition 1: Retention time=1.99 min. ESI-MS (+) m/z [M+2H]²⁺: 1032.2.

### Preparation of Example 327

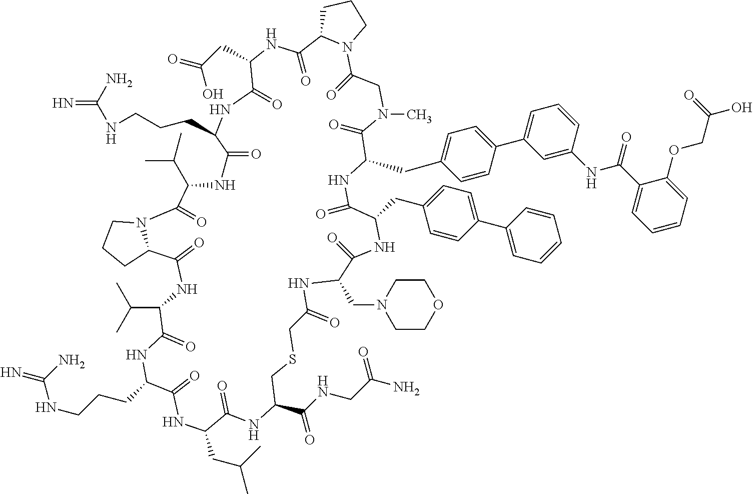 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3cccc(NC(=O)c4ccccc4OCC(=O)O)c3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 327 was prepared on a 50 Preparative LC/MS. The yield of the product was 7.5 mg, and its estimated purity by LCMS analysis was 100%. Analysis condition 1: Retention time=1.37 min. ESI-MS (+) m/z [M+2H]²⁺:1009.7.

### Preparation of Example 328

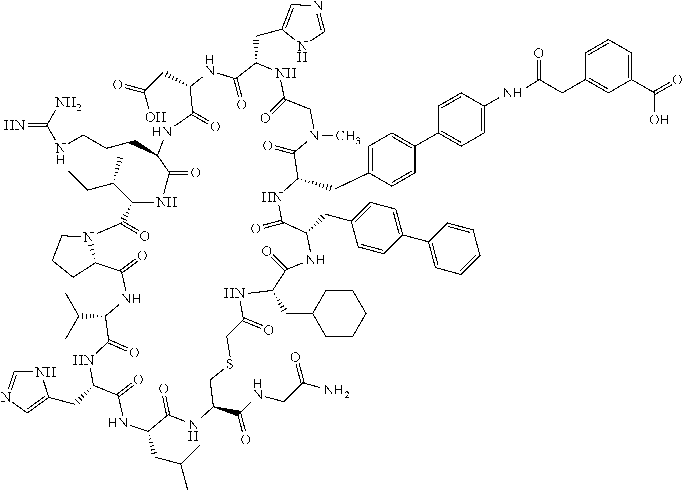 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)Cc4cccc(C(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

### Step 1: Synthesis of the Linear Sequence on the Prelude Synthesizer

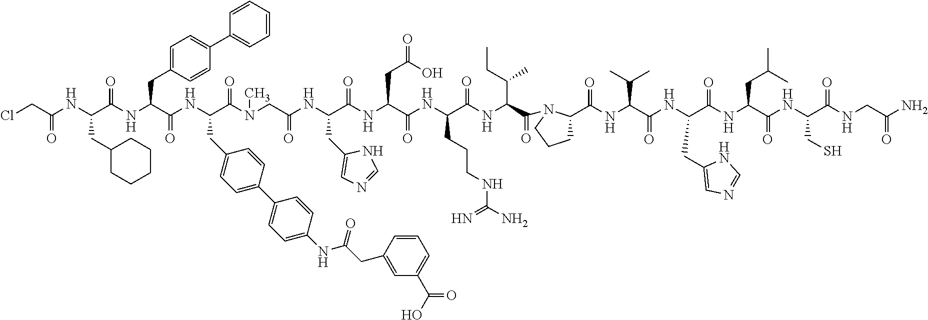 [SMILES: CC[C@H](C)[C@H](NC(=O)[C@@H](CCCNC(=N)N)NC(=O)[C@H](CC(=O)O)NC(=O)[C@H](Cc1cnc[nH]1)NC(=O)CN(C)C(=O)[C@H](Cc1ccc(-c2ccc(NC(=O)Cc3cccc(C(=O)O)c3)cc2)cc1)NC(=O)[C@H](Cc1ccc(-c2ccccc2)cc1)NC(=O)[C@H](CC1CCCCC1)NC(=O)CCl)C(=O)N1CCC[C@H]1C(=O)N[C@H](C(=O)N[C@@H](Cc1cnc[nH]1)C(=O)N[C@@H](CC(C)C)C(=O)N[C@@H](CS)C(=O)NCC(N)=O)C(C)C]

To the 4×45-mL reactors 0.20 mmol of Fmoc-Rink Amide Resin (0.56 mmol/g, 357.2 mg) was placed on the Prelude synthesizer. The following procedures were then performed sequentially:
“Resin-swelling procedure” was followed;
“Single-coupling procedure” was followed with Fmoc-Gly-OH;
“Single-coupling procedure” was followed with Fmoc-Cys (Trt)-OH;
“Single-coupling procedure” was followed with Fmoc-Leu-OH;
“Single-coupling procedure” was followed with Fmoc-His (Trt)-OH;
“Single-coupling procedure” was followed with Fmoc-Val-OH;
“Single-coupling procedure” was followed with Fmoc-Pro-OH;
“Single-coupling procedure” was followed with Fmoc-Ile-OH;
“Single-coupling procedure” was followed with Fmoc-d-Arg (Pbf)-OH;
“Single-coupling procedure” was followed with Fmoc-Asp (OtBu)-OH;
“Single-coupling procedure” was followed with Fmoc-His (Trt)-OH;
“Single-coupling procedure” was followed with Fmoc-mGly-OH;
“Single-coupling procedure” was followed with Fmoc-Bip (4′-NH-Dde)-OH

To the resulting resin (0.20 mmol) was added a solution of NH₂OH—HCl (1.25 gram, 1.8 mmol) and imidazole (0.918 gram, 1.35 mmol) in NMP/DMF (5:1, 6 mL). The resin was shaken for 40 minutes, washed with DMF (6×5 mL).

To a portion of the resin (0.04 mmol) was added a solution of 2-[3-(methoxycarbonyl)phenyl] acetic acid (31.1 mg, 0.160 mmol) in DMF (0.5 mL), a solution of 0.2M HATU in DMF (800 ul, 0.160 mmol) followed by 0.4M NMM in DMF (800 ul, 0.32 mmol). The reaction was shaken overnight, and the resin was washed with DMF (6×1 mL) on the instrument.

The following procedures were then performed sequentially:
“Single-coupling procedure” was followed with Fmoc-Bip-OH;
“Single-coupling procedure” was followed with Fmoc-Cha-OH;
“Chloroacetic Anhydride Coupling”

Finally, the resin bound peptide was cleaved off the resin and deprotected following Global Deprotection Method procedure to provide crude linear peptide.

### Step 2. Macrocyclization:

To the crude peptide from the previous step was added 40 mL of 10% DIEPA in DMF (40 mL). The reaction mixture was shaken overnight, filtered through a 0.45-micron filter, and concentrated to dryness.

The crude material was purified via preparative LC/MS with the following conditions: Column: Waters XBridge C18, 19×250 mm, 5-μm particles; Mobile Phase A: 5:95 acetonitrile:water with 0.1% TFA; Mobile Phase B: 95:5 acetonitrile:water with 0.1% TFA; Gradient: 14-44% B over 20 minutes, and 0-minute hold at 100% B; Flow: 20 mL/min. Fractions containing the desired product were combined and dried via centrifugal evaporation. It provided product, 3 mg. Its estimated purity by LCMS analysis was 91.4%.
Analysis condition 2: Retention time=1.47 min. ESI-MS(+) m/z [M+2H]²⁺: 1017.2.

Examples: 305, 308, 309, 315-319, 322-326, 327-332, 328-331, 342, 344, 345, 349-354, 367-381, 383, 395, 399-402, 411-422, 442, 450, 481-497, 502, 503, 513-516, 521, 523, 525, 526, 527, 528, 529, 530, 573.-576, 586-597, 606-618, 630-634 were prepared following analogous procedures described for Example 328.

### Preparation of Example 329

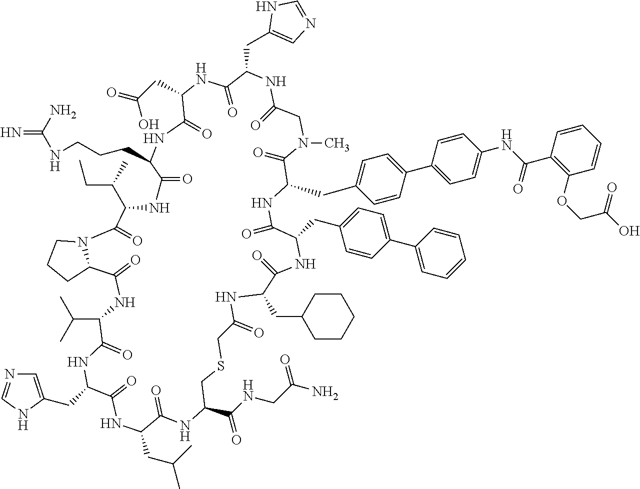 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccccc4OCC(=O)O)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 329 was prepared on a 50 umol scale. It was purified by Preparative LC/MS. The yield of the product was 19.3 mg, and its estimated purity by LCMS analysis was 96.8%. Analysis condition 2: Retention time=1.54 min. ESI-MS (+) m/z [M+2H]²⁺: 1025.4.

### Preparation of Example 330

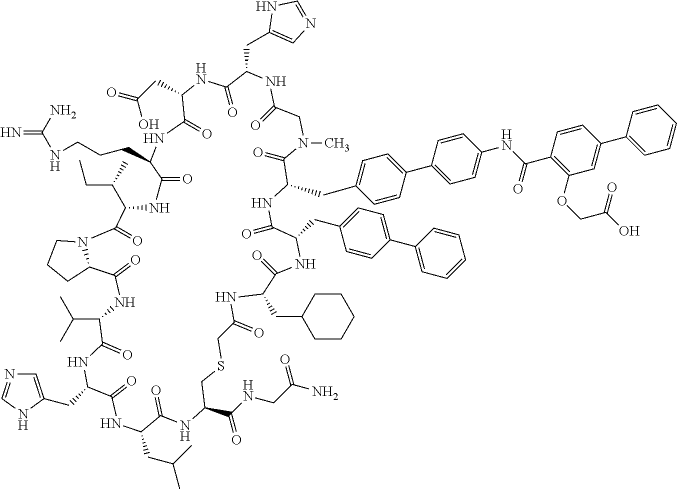 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccc(-c5ccccc5)cc4OCC(=O)O)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 330 was prepared on a 50 Preparative LC/MS. The yield of the product was 20.3 mg, and its estimated purity by LCMS analysis was 100%. Analysis condition 2: Retention time=1.65 min. ESI-MS (+) m/z [M+2H]²⁺: 1063.6.

### Preparation of Example 331

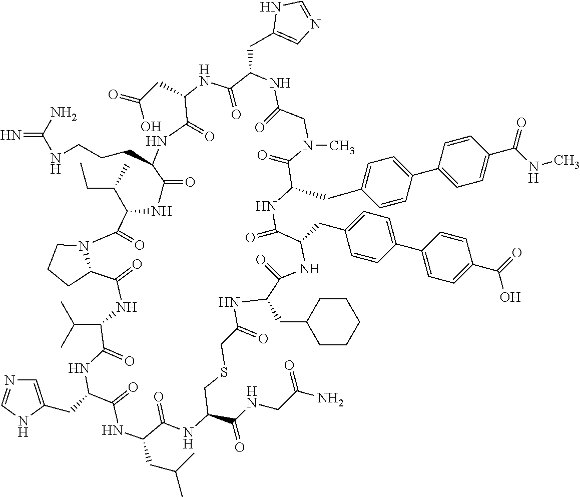 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(C(=O)NC)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 331 was prepared on a 50 umol scale. It was purified by Preparative LC/MS. The yield of the product was 24.9 mg, and its estimated purity by LCMS analysis was 94.7%. Analysis condition 2: Retention time=1.3 min. ESI-MS (+) m/z [M+2H]²⁺: 979.4.

### Preparation of Example 332

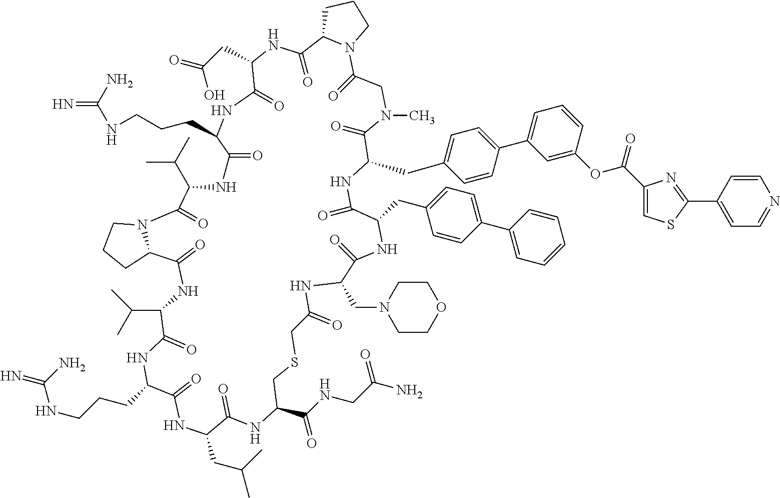 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3cccc(OC(=O)c4csc(-c5ccncc5)n4)c3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 332 was prepared on a 50 Preparative LC/MS. The yield of the product was 6.7 mg, and its estimated purity by LCMS analysis was 100%. Analysis condition 21: Retention time=1.5 min. ESI-MS (+) m/z [M+2H]²⁺: 1014.4.

### Preparation of Example 333

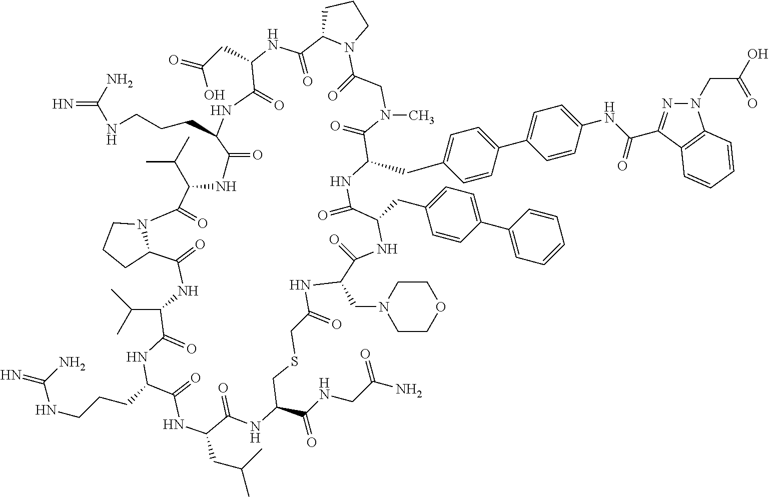 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 333 was prepared on a 50 umol scale. It was purified by Preparative LC/MS. The yield of the product was 19.4 mg, and its estimated purity by LCMS analysis was 100%. Analysis condition 2: Retention time=1.45 min. ESI-MS (+) m/z [M+2H]²⁺: 1021.6.

### Preparation of Example 334

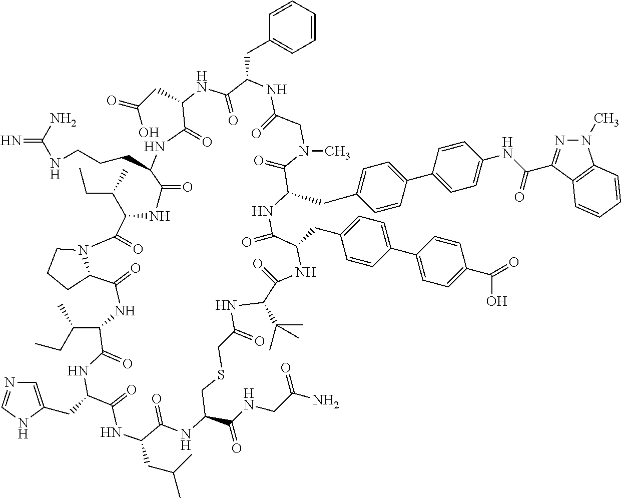 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccc2)NC(=O)CN(C)C(=O)[C@H](Cc2ccc(-c3ccc(NC(=O)c4nn(C)c5ccccc45)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cnc[nH]2)NC1=O]

Example 334 was prepared on a 50 Preparative LC/MS. The yield of the product was 5.5 mg, and its estimated purity by LCMS analysis was 90.8%. Analysis condition 1: Retention time=1.58 min. ESI-MS (+) m/z [M+2H]²⁺: 1029.

### Preparation of Example 335

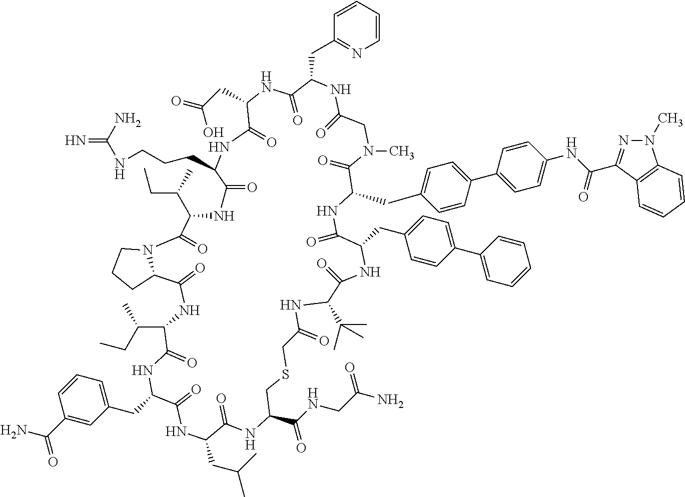 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cccc(C(N)=O)c2)NC1=O]

Example 335 was prepared on a 50 umol scale. It was purified by Preparative LC/MS. The yield of the product was 2 mg, and its estimated purity by LCMS analysis was 93.6%. Analysis condition 2: Retention time=1.65 min. ESI-MS(+) m/z [M+2H]²⁺: 1034.6.

### Preparation of Example 336

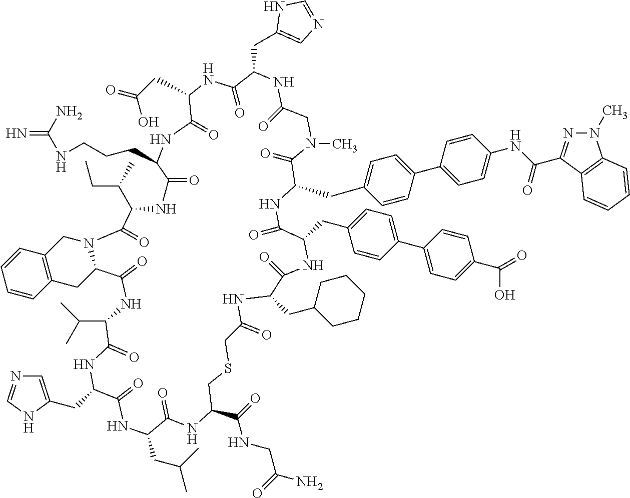 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4nn(C)c5ccccc45)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2Cc3ccccc3CN2C1=O]

Example 336 was prepared on a 50 Preparative LC/MS. The yield of the product was 2.2 mg, and its estimated purity by LCMS analysis was 96.6%. Analysis condition 2: Retention time=1.59 min. ESI-MS (+) m/z [M+2H]²⁺: 1068.4.

### Preparation of Example 337

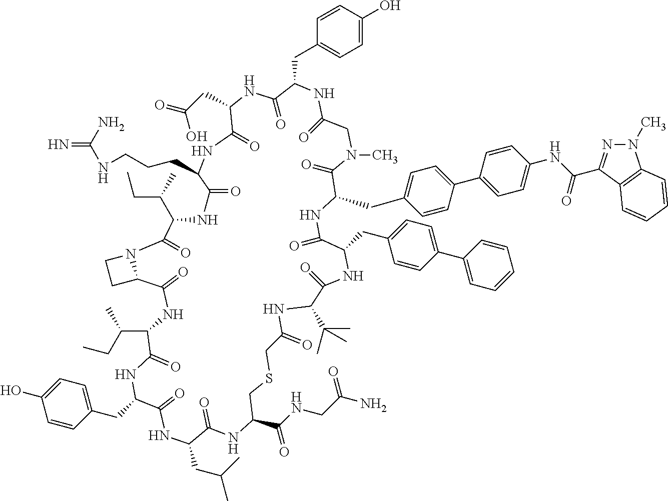 [SMILES: CC[C@H](C)[C@@H]1NC(=O)[C@@H]2CCN2C(=O)[C@H]([C@@H](C)CC)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2ccc(O)cc2)NC1=O]

Example 337 was prepared on a 50 umol scale. It was purified by Preparative LC/MS. The yield of the product was 4.3 mg, and its estimated purity by LCMS analysis was 90.3%. Analysis condition 1: Retention time=1.62 min. ESI-MS (+) m/z [M+2H]²⁺: 1021.6.

### Preparation of Example 338

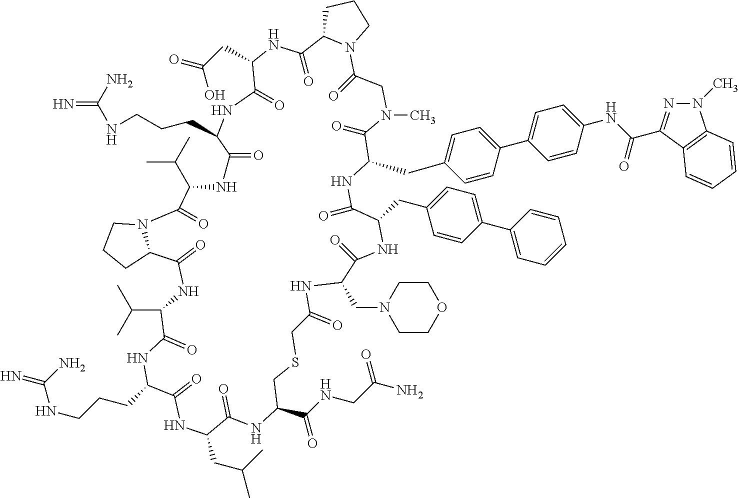 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 338 was prepared on a 50 Preparative LC/MS. The yield of the product was 1.6 mg, and its estimated purity by LCMS analysis was 100%. Analysis condition 1: Retention time=1.68 min. ESI-MS (+) m/z [M+H]⁺: 1997.2.

### Preparation of Example 339

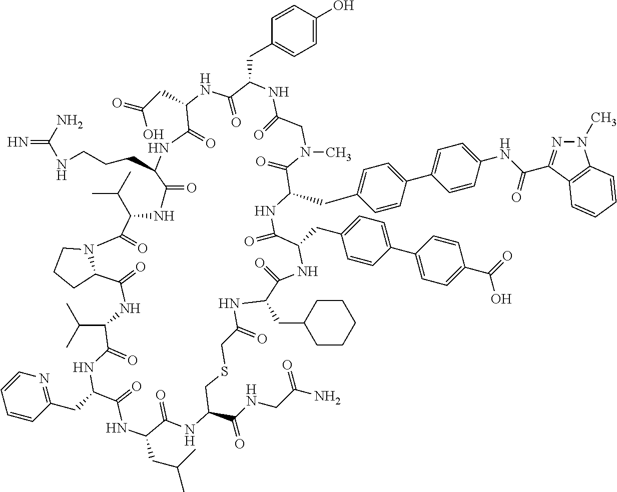

Example 339 was prepared on a 50 umol scale. It was purified by Preparative LC/MS. The yield of the product was 1.6 mg, and its estimated purity by LCMS analysis was 97.2%. Analysis condition 2: Retention time=1.65 min. ESI-MS (+) m/z [M+2H]²⁺: 1048.9.

### Preparation of Example 340

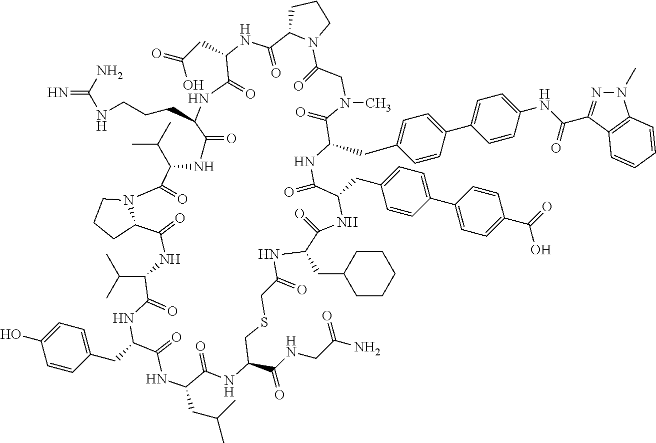 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4nn(C)c5ccccc45)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 340 was prepared on a 50 Preparative LC/MS. The yield of the product was 4.8 mg, and its estimated purity by LCMS analysis was 90%. Analysis condition 1: Retention time=1.46 min. ESI-MS (+) m/z [M+2H]²⁺: 1023.5.

### Preparation of Example 341

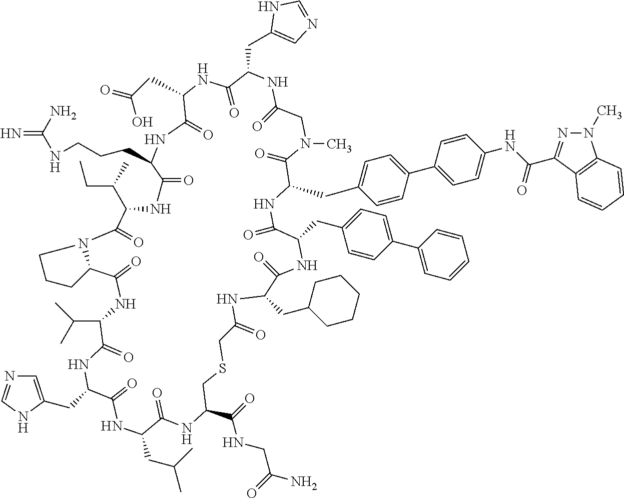 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 341 was prepared on a 50 umol scale. It was purified by Preparative LC/MS. The yield of the product was 1.7 mg, and its estimated purity by LCMS analysis was 88.2%. Analysis condition 2: Retention time=1.59 min. ESI-MS (+) m/z [M+2H]²⁺: 1015.4.

### Preparation of Example 342

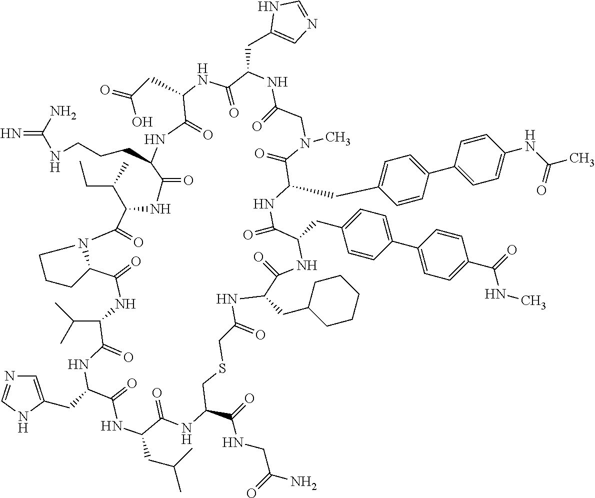 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(C)=O)cc3)cc2)NC(=O)[C@H](Cc2ccc(-c3ccc(C(=O)NC)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 342 was prepared on a 50 umol scale. It was purified by Preparative LC/MS. The yield of the product was 18.2 mg, and its estimated purity by LCMS analysis was 97.3%. Analysis condition 1: Retention time=1.28 min. ESI-MS(+) m/z [M+H]⁺: 1969.9.

### Preparation of Example 343

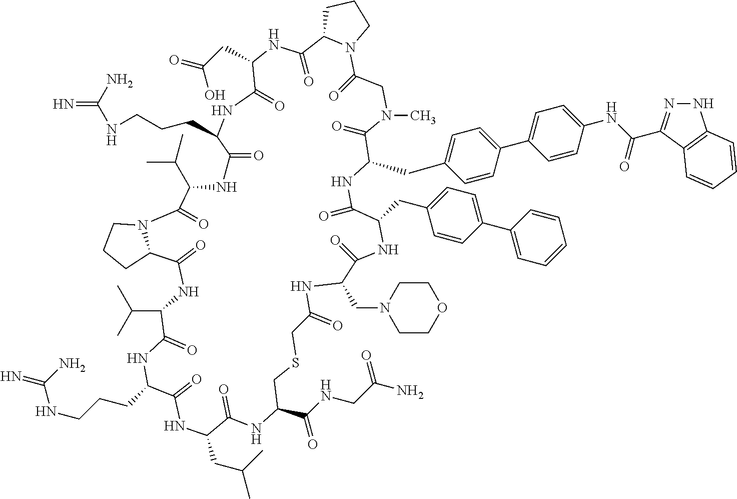 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4n[nH]c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 343 was prepared on a 8 umol scale. It was purified by Preparative LC/MS. The yield of the product was 14.9 mg, and its estimated purity by LCMS analysis was 100%. Analysis condition 2: Retention time=1.44 min. ESI-MS (+) m/z [M+H]⁺: 1983.6.

### Preparation of Example 344

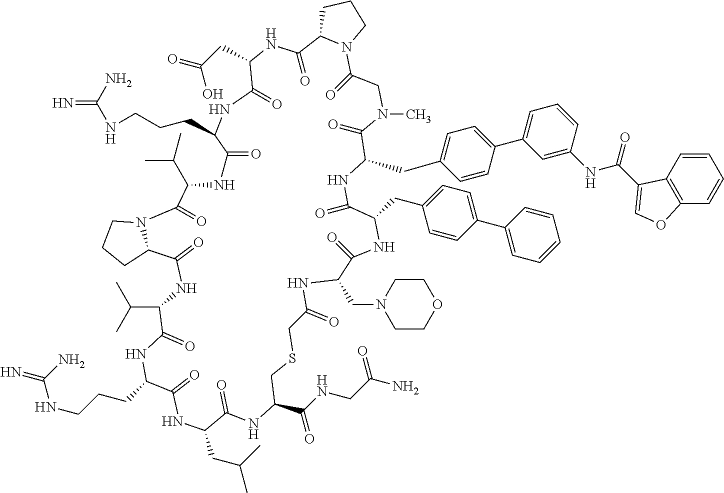 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3cccc(NC(=O)c4coc5ccccc45)c3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 344 was prepared on a 50 Preparative LC/MS. The yield of the product was 1.3 mg, and its estimated purity by LCMS analysis was 95.7%. Analysis condition 1: Retention time=1.63 min. ESI-MS (+) m/z [M+H]⁺: 1983.3.

### Preparation of Example 345

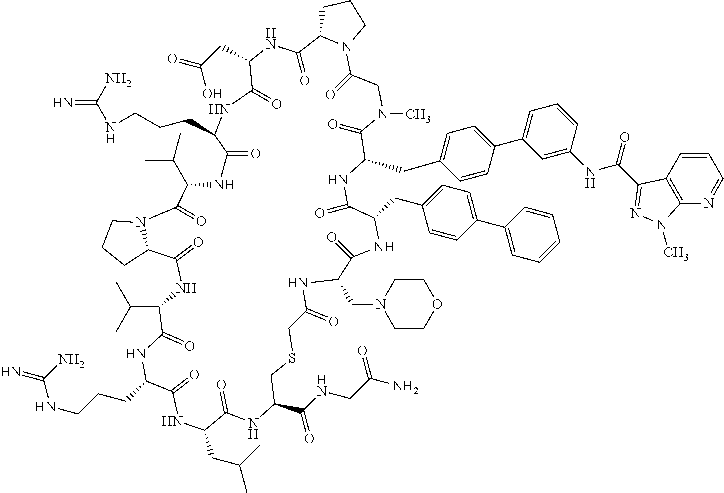 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3cccc(NC(=O)c4nn(C)c5ncccc45)c3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 345 was prepared on a 50 umol scale. It was purified by Preparative LC/MS. The yield of the product was 2.2 mg, and its estimated purity by LCMS analysis was 93%. Analysis condition 2: Retention time=1.45 min. ESI-MS (+) m/z [M+H]⁺: 1999.

### Preparation of Example 346

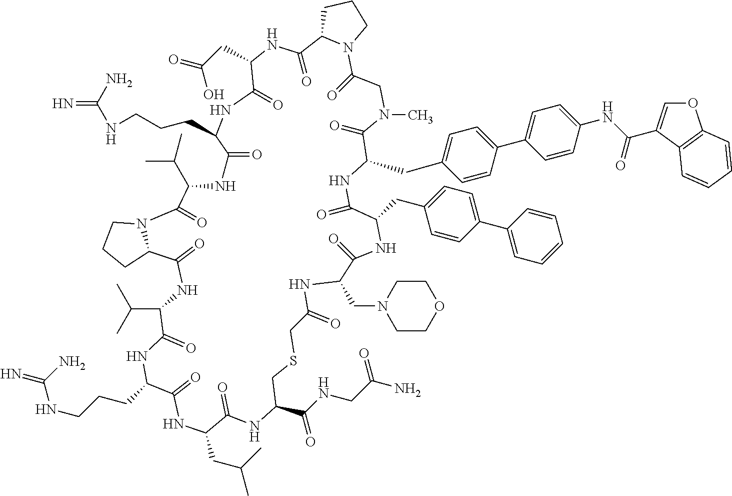 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4coc5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 346 was prepared on a 8 Preparative LC/MS. The yield of the product was 10.7 mg, and its estimated purity by LCMS analysis was 87.7%. Analysis condition 1: Retention time=1.55 min. ESI-MS (+) m/z [M+H]⁺: 1983.

### Preparation of Example 347

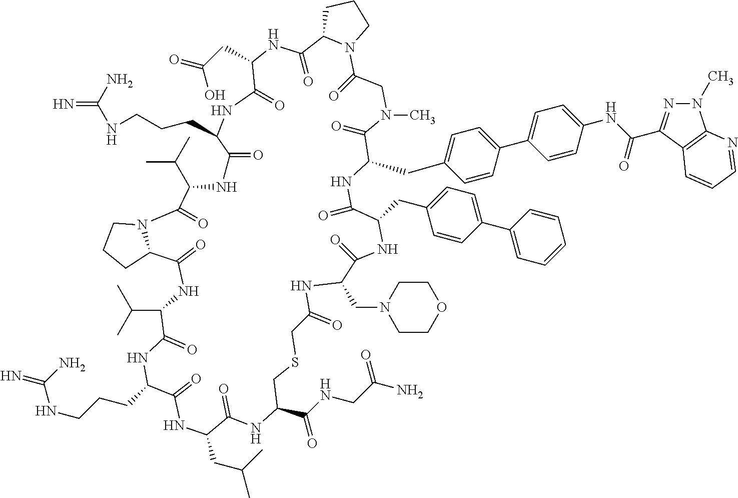 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4nn(C)c5ncccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 347 was prepared on a 8 umol scale. It was purified by Preparative LC/MS. The yield of the product was 10.9 mg, and its estimated purity by LCMS analysis was 90.9%. Analysis condition 2: Retention time=1.46 min. ESI-MS (+) m/z [M+H]⁺: 1998.5.

### Preparation of Example 348

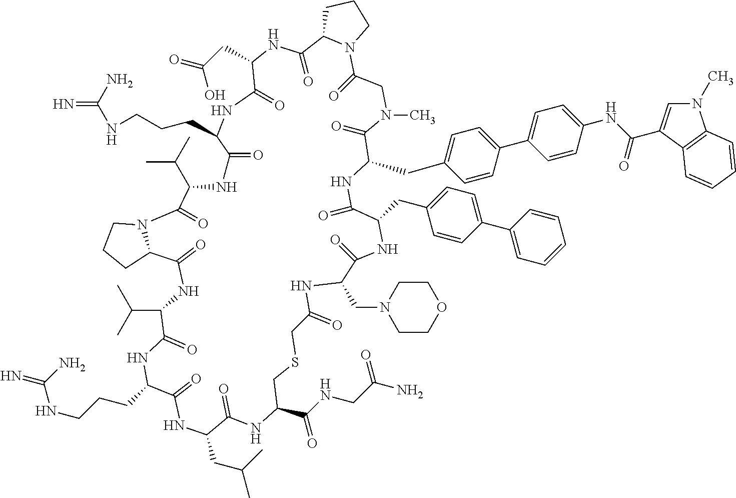 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4cn(C)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 348 was prepared on a 50 Preparative LC/MS. The yield of the product was 11.9 mg, and its estimated purity by LCMS analysis was 100%. Analysis condition 2: Retention time=1.5 min. ESI-MS (+) m/z [M+H]⁺: 1995.9.

### Preparation of Example 349

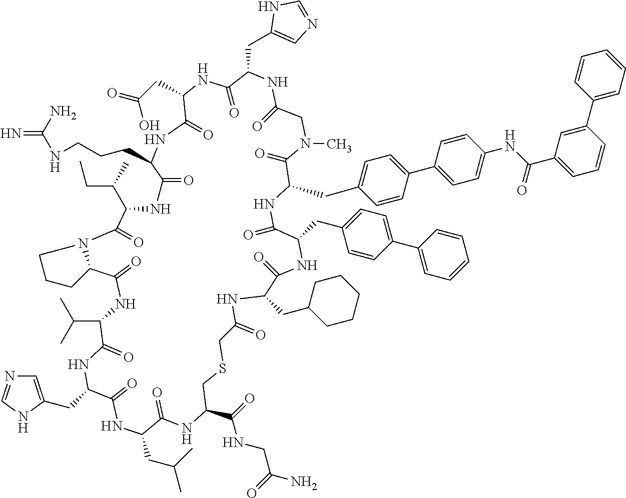 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(-c5ccccc5)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 349 was prepared on a 50 umol scale. It was purified by Preparative LC/MS. The yield of the product was 25.6 mg, and its estimated purity by LCMS analysis was 100%. Analysis condition 2: Retention time=1.66 min. ESI-MS (+) m/z [M+2H]²⁺: 1026.7.

### Preparation of Example 350

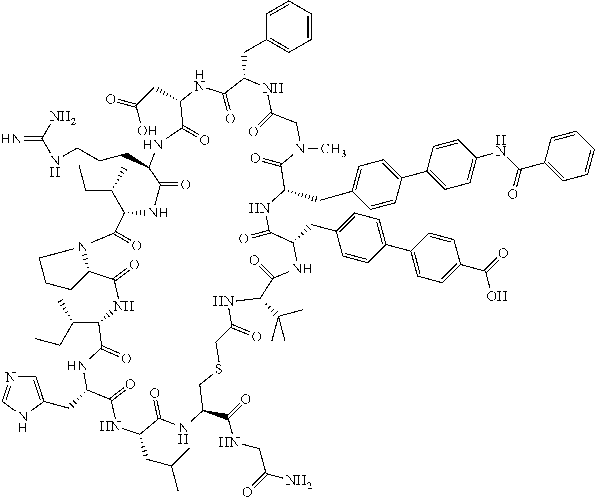 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccc2)NC(=O)CN(C)C(=O)[C@H](Cc2ccc(-c3ccc(NC(=O)c4ccccc4)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cnc[nH]2)NC1=O]

Example 350 was prepared on a 50 Preparative LC/MS. The yield of the product was 16 mg, and its estimated purity by LCMS analysis was 95.2%. Analysis condition 1: Retention time=1.35 min. ESI-MS(+) m/z [M+2H]²⁺: 1002.6.

### Preparation of Example 351

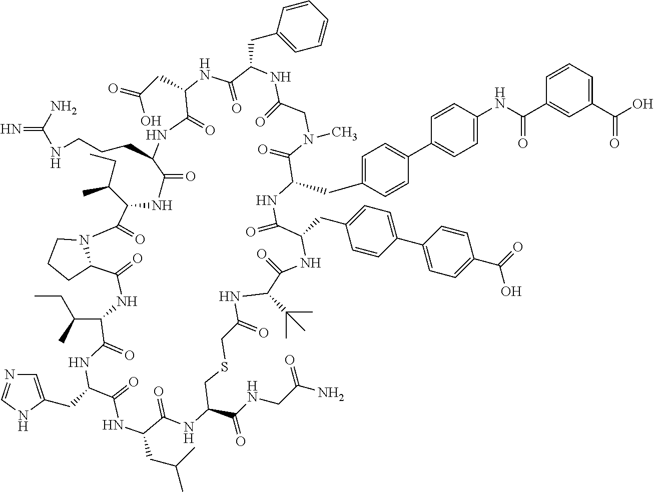 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccc2)NC(=O)CN(C)C(=O)[C@H](Cc2ccc(-c3ccc(NC(=O)c4cccc(C(=O)O)c4)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cnc[nH]2)NC1=O]

Example 351 was prepared on a 50 umol scale. It was purified by Preparative LC/MS. The yield of the product was 20.4 mg, and its estimated purity by LCMS analysis was 100%. Analysis condition 2: Retention time=1.42 min. ESI-MS(+) m/z [M+2H] 2+: 1024.6.

### Preparation of Example 352

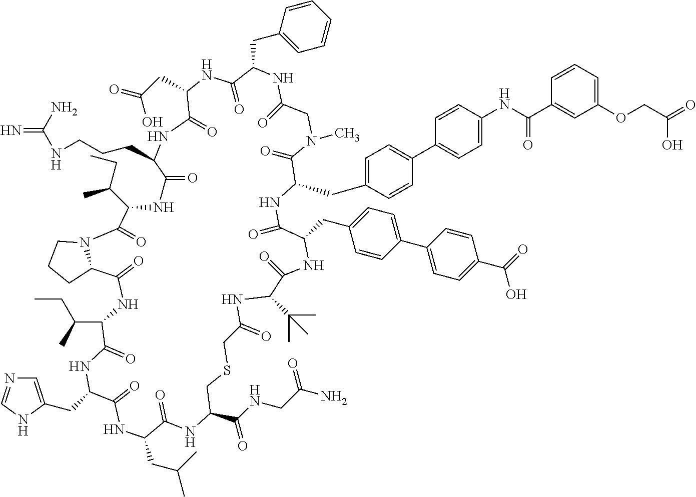 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccc2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cnc[nH]2)NC1=O]

Example 352 was prepared on a 50 Preparative LC/MS. The yield of the product was 17.1 mg, and its estimated purity by LCMS analysis was 99%. Analysis condition 1: Retention time=1.13 min. ESI-MS(+) m/z [M+2H] 2+: 1039.6.

### Preparation of Example 353

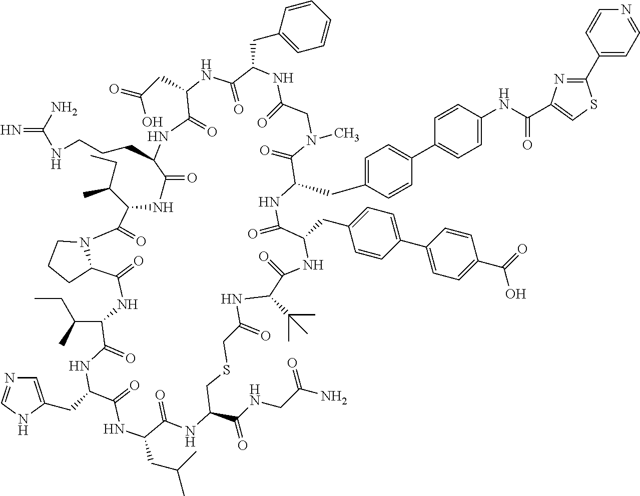 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccc2)NC(=O)CN(C)C(=O)[C@H](Cc2ccc(-c3ccc(NC(=O)c4csc(-c5ccncc5)n4)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cnc[nH]2)NC1=O]

Example 353 was prepared on a 50 umol scale. It was purified by Preparative LC/MS. The yield of the product was 22.1 mg, and its estimated purity by LCMS analysis was 98.4%. Analysis condition 1: Retention time=1.34 min. ESI-MS(+) m/z [M+2H] 2+: 1044.7.

### Preparation of Example 354

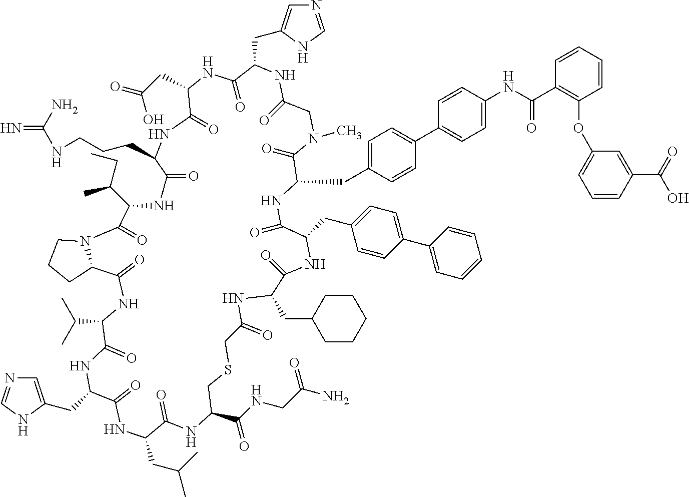 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccccc4Oc4cccc(C(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 354 was prepared on a 50 umol scale. It was purified by Preparative LC/MS. The yield of the product was 16.8 mg, and its estimated purity by LCMS analysis was 100%. Analysis condition 2: Retention time=1.48 min. ESI-MS(+) m/z [M+2H] 2+: 1056.7.

### Preparation of Example 355

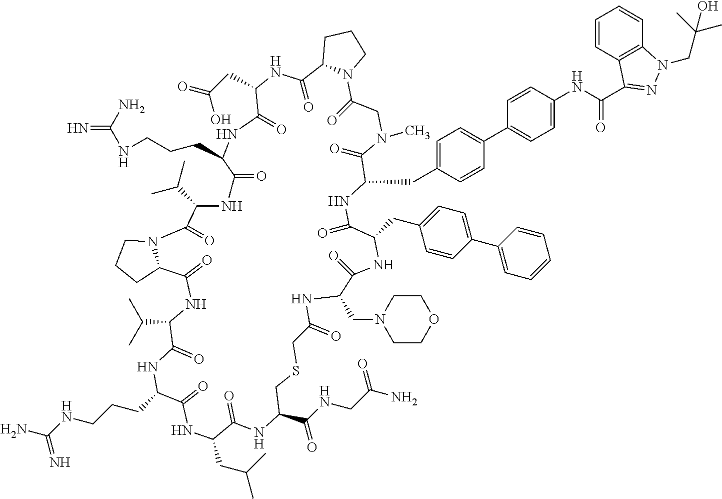 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4nn(CC(C)(C)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 355 was prepared on a 27 umol scale. It was purified by Preparative LC/MS. The yield of the product was 1.8 mg, and its estimated purity by LCMS analysis was 94%. Analysis condition 2: Retention time=1.49 min. ESI-MS(+) m/z [M+3H] 3+:

### Preparation of Example 356

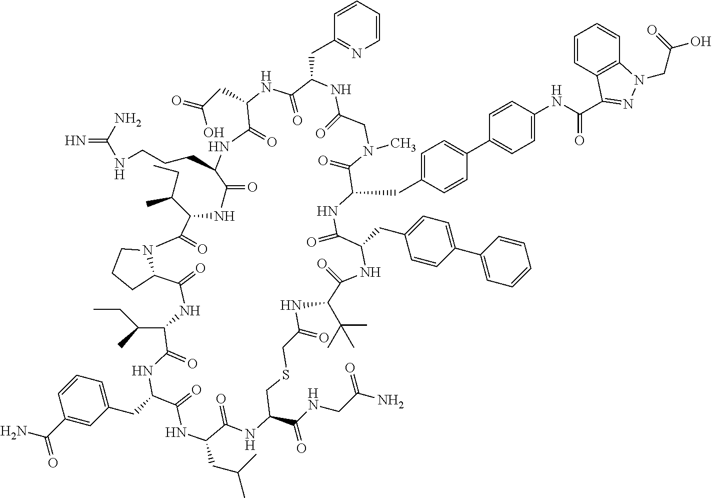 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cccc(C(N)=O)c2)NC1=O]

Example 356 was prepared on a 50 Preparative LC/MS. The yield of the product was 2 mg, and its estimated purity by LCMS analysis was 90%. Analysis condition 2: Retention time=1.64 min. ESI-MS(+) m/z [M+2H] 2+: 1056.5.

### Preparation of Example 357

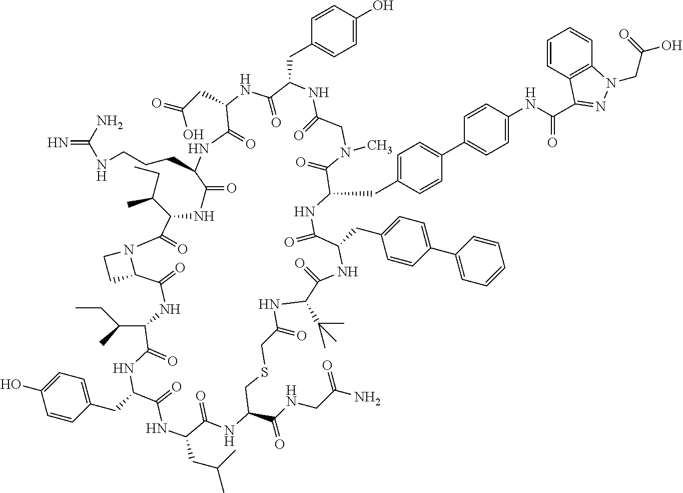 [SMILES: CC[C@H](C)[C@@H]1NC(=O)[C@@H]2CCN2C(=O)[C@H]([C@@H](C)CC)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2ccc(O)cc2)NC1=O]

Example 357 was prepared on a 50 umol scale. It was purified by Preparative LC/MS. The yield of the product was 14.8 mg, and its estimated purity by LCMS analysis was 90.4%. Analysis condition 2: Retention time=1.64 min. ESI-MS(+) m/z [M+2H] 2+: 1043.5.

### Preparation of Example 358

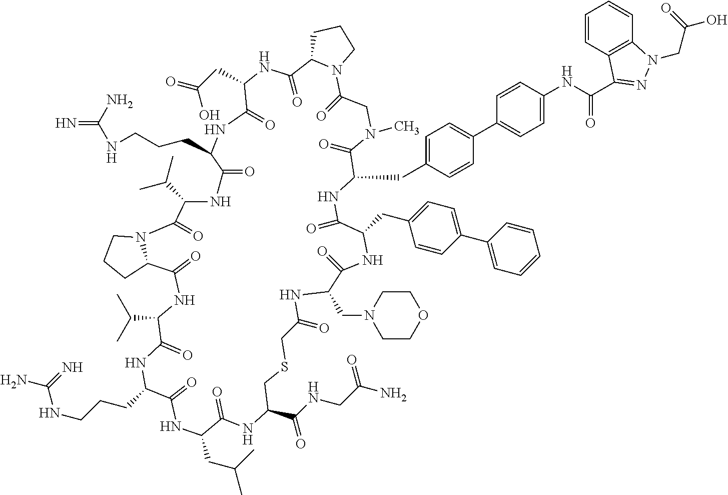 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 358 was prepared on a 50 Preparative LC/MS. The yield of the product was 7.5 mg, and its estimated purity by LCMS analysis was 100%. Analysis condition 2: Retention time=1.52 min. ESI-MS(+) m/z [M+2H] 2+: 1021.7.

### Preparation of Example 359

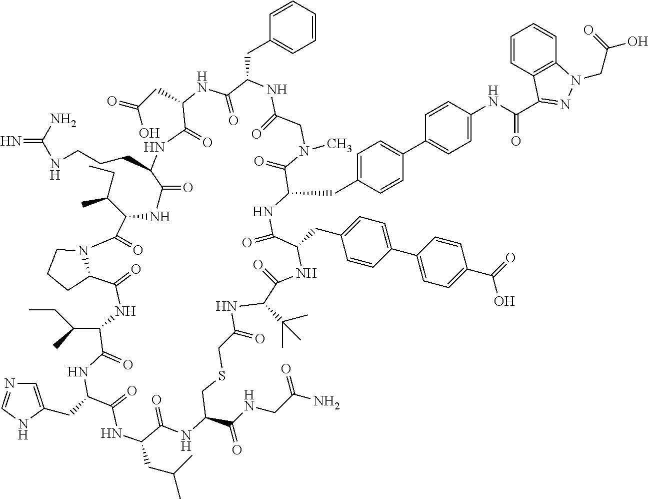 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccc2)NC(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cnc[nH]2)NC1=O]

Example 359 was prepared on a 50 umol scale. It was purified by Preparative LC/MS. The yield of the product was 10.6 mg, and its estimated purity by LCMS analysis was 90.1%. Analysis condition 1: Retention time=1.24 min. ESI-MS(+) m/z [M+2H] 2+: 1051.5.

### Preparation of Example 360

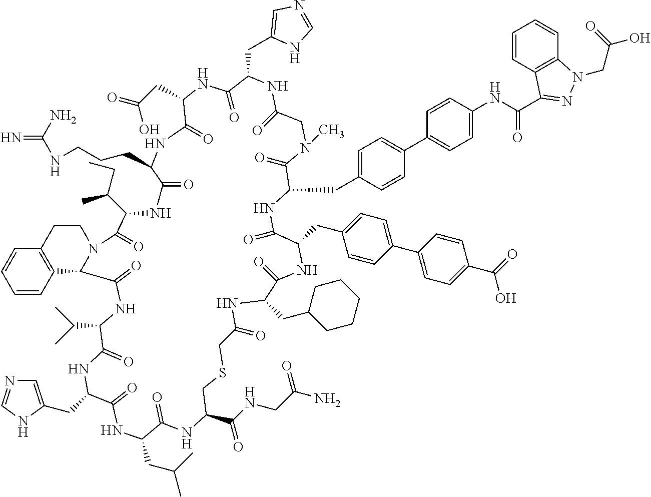 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2c3ccccc3CCN2C1=O]

Example 360 was prepared on a 50 Preparative LC/MS. The yield of the product was 36.2 mg, and its estimated purity by LCMS analysis was 100%. Analysis condition 1: Retention time=1.39 min. ESI-MS(+) m/z [M+2H] 2+: 1090.5.

### Preparation of Example 361

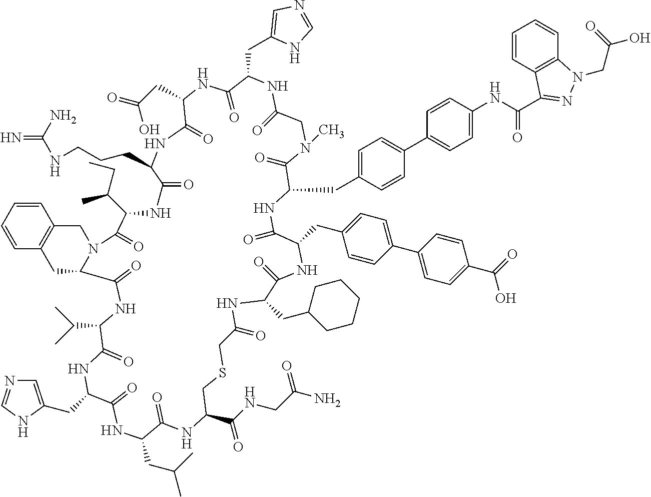 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2Cc3ccccc3CN2C1=O]

Example 361 was prepared on a 50 umol scale. It was purified by Preparative LC/MS. The yield of the product was 11.3 mg, and its estimated purity by LCMS analysis was 97.5%. Analysis condition 1: Retention time=1.44 min. ESI-MS(+) m/z [M+2H] 2+: 1090.

### Preparation of Example 362

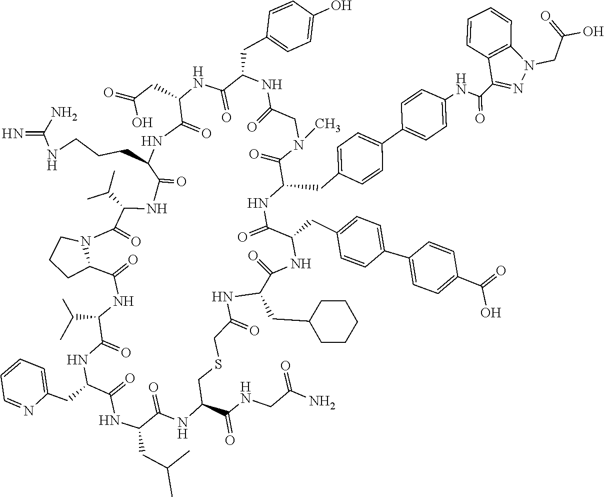 [SMILES: CC(C)C[C@@H]1NC(=O)[C@H](Cc2ccccn2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 362 was prepared on a 50 Preparative LC/MS. The yield of the product was 5.7 mg, and its estimated purity by LCMS analysis was 96.3%. Analysis condition 1: Retention time=1.28 min. ESI-MS(+) m/z [M+2H] 2+: 1071.

### Preparation of Example 363

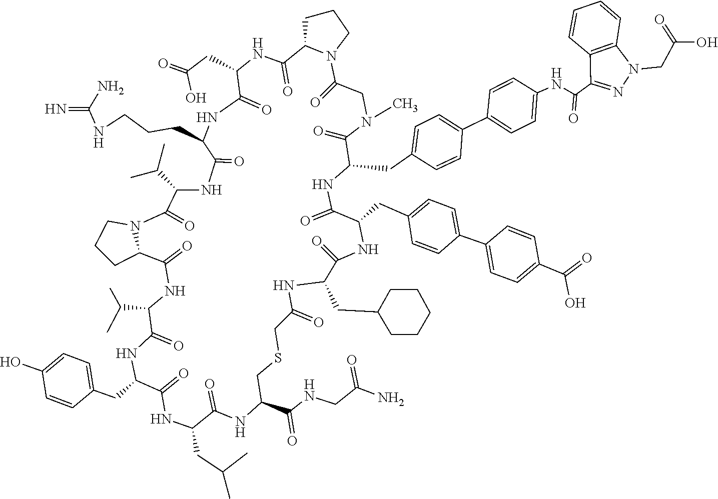 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 363 was prepared on a 50 umol scale. It was purified by Preparative LC/MS. The yield of the product was 4.2 mg, and its estimated purity by LCMS analysis was 92%. Analysis condition 1: Retention time=1.17 min. ESI-MS(+) m/z [M+2H] 2+: 1045.4.

### Preparation of Example 364

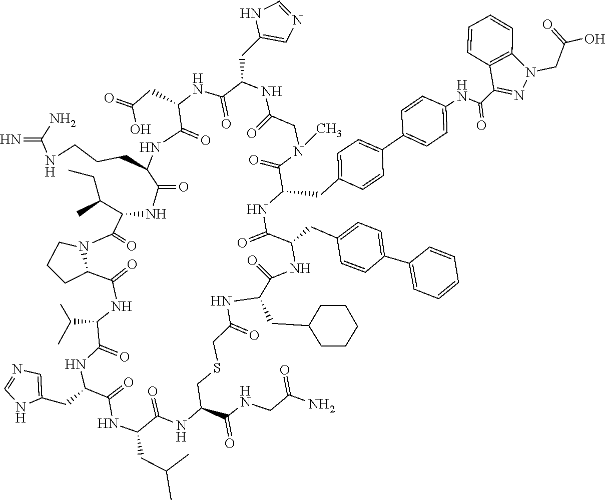 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 364 was prepared on a 50 Preparative LC/MS. The yield of the product was 9.1 mg, and its estimated purity by LCMS analysis was 100%. Analysis condition 2: Retention time=1.47 min. ESI-MS(+) m/z [M+2H] 2+: 1037.5.

### Preparation of Example 365

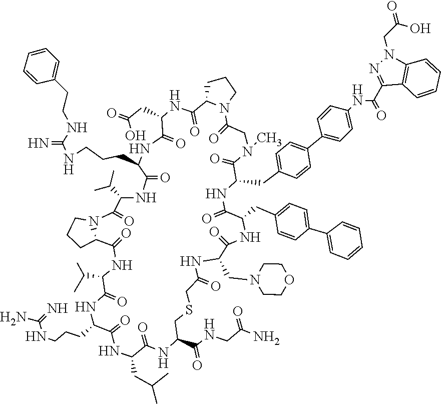 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCc2ccccc2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 365 was prepared on a 50 umol scale. It was purified by Preparative LC/MS. The yield of the product was 12.1 mg, and its estimated purity by LCMS analysis was 89.8%. Analysis condition 1: Retention time=1.53 min. ESI-MS(+) m/z [M+2H] 2+: 1073.6.

### Preparation of Example 366

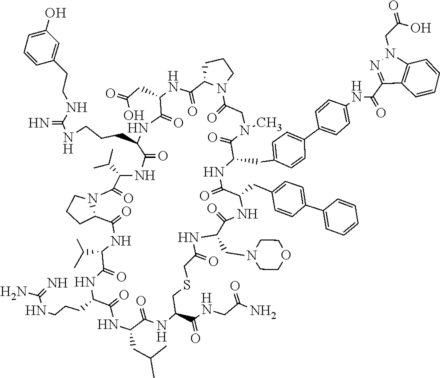 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCc2cccc(O)c2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 366 was prepared on a 100 Preparative LC/MS. The yield of the product was 3.1 mg, and its estimated purity by LCMS analysis was 90.1%. Analysis condition 2: Retention time=1.49 min. ESI-MS(+) m/z [M+2H] 2+: 1081.5.

### Preparation of Example 367

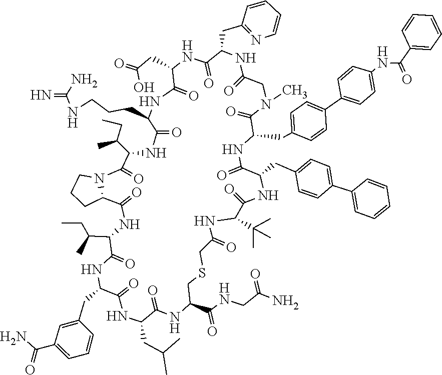 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(=O)c4ccccc4)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cccc(C(N)=O)c2)NC1=O]

Example 367 was prepared on a 50 umol scale. It was purified by Preparative LC/MS. The yield of the product was 11 mg, and its estimated purity by LCMS analysis was 100%. Analysis condition 2: Retention time=1.56 min. ESI-MS(+) m/z [M+2H] 2+: 1007.

### Preparation of Example 368

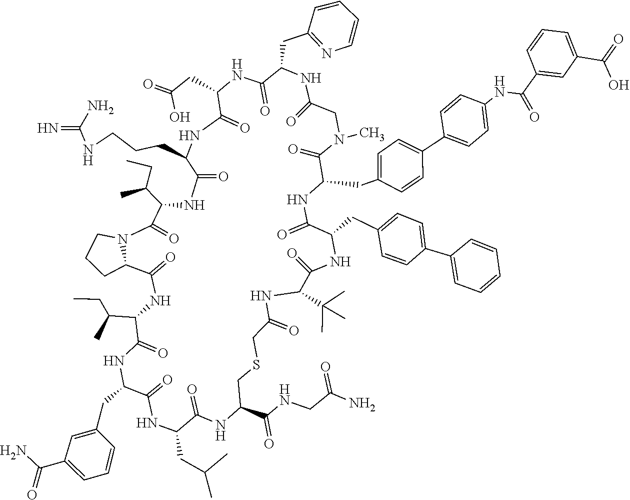 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(=O)c4cccc(C(=O)O)c4)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cccc(C(N)=O)c2)NC1=O]

Example 368 was prepared on a 50 umol scale. It was purified by Preparative LC/MS. The yield of the product was 17.3 mg, and its estimated purity by LCMS analysis was 100%. Analysis condition 2: Retention time=1.42 min. ESI-MS(+) m/z [M+2H] 2+: 1029.7.

### Preparation of Example 369

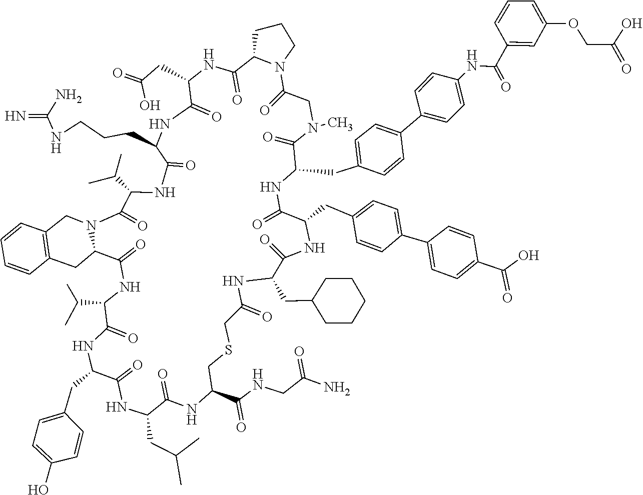 [SMILES: CC(C)C[C@@H]1NC(=O)[C@H](Cc2ccc(O)cc2)NC(=O)[C@H](C(C)C)NC(=O)[C@@H]2Cc3ccccc3CN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 369 was prepared on a 50 umol scale. It was purified by Preparative LC/MS. The yield of the product was 11.6 mg, and its estimated purity by LCMS analysis was 100%. Analysis condition 2: Retention time=1.49 min. ESI-MS(+) m/z [M+2H] 2+: 1064.5

### Preparation of Example 370

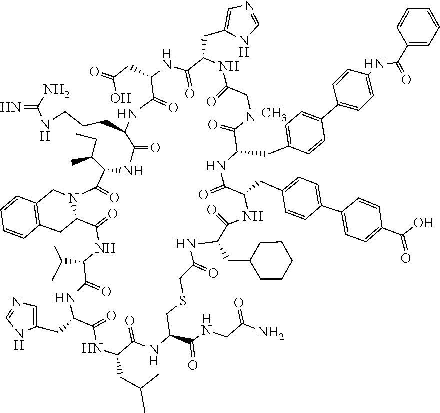 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccccc4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2Cc3ccccc3CN2C1=O]

Example 370 was prepared on a 50 Preparative LC/MS. The yield of the product was 15.4 mg, and its estimated purity by LCMS analysis was 100%. Analysis condition 2: Retention time=1.44 min. ESI-MS(+) m/z [M+2H] 2+: 1041.6.

### Preparation of Example 371

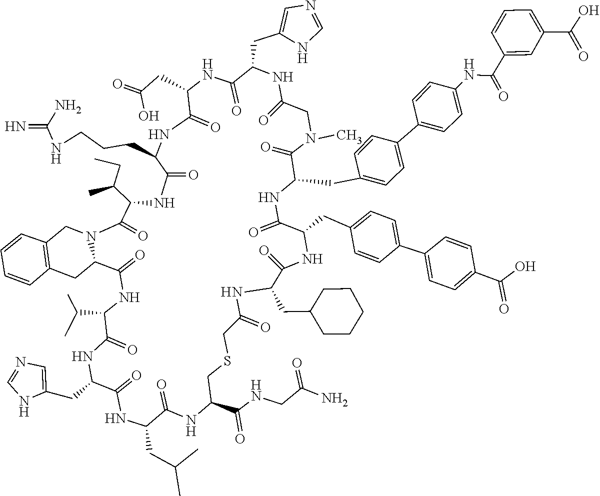 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(C(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2Cc3ccccc3CN2C1=O]

Example 371 was prepared on a 50 umol scale. It was purified by Preparative LC/MS. The yield of the product was 6.6 mg, and its estimated purity by LCMS analysis was 96.6%. Analysis condition 2: Retention time=1.43 min. ESI-MS(+) m/z [M+2H] 2+: 1063.5.

### Preparation of Example 372

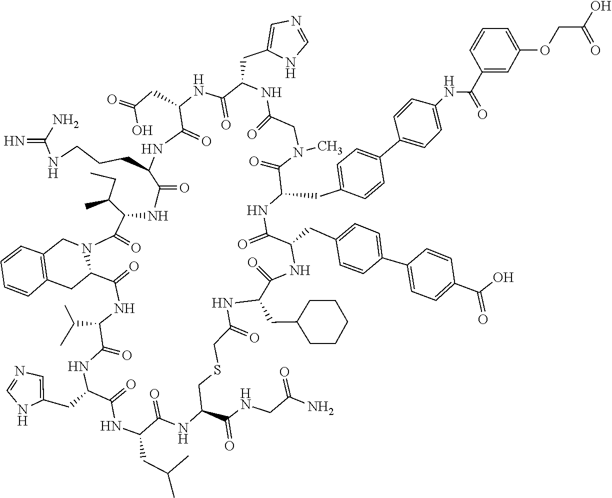 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2Cc3ccccc3CN2C1=O]

Example 372 was prepared on a 50 Preparative LC/MS. The yield of the product was 11.7 mg, and its estimated purity by LCMS analysis was 100%. Analysis condition 2: Retention time=1.34 min. ESI-MS(+) m/z [M+2H] 2+: 1078.7.

### Preparation of Example 373

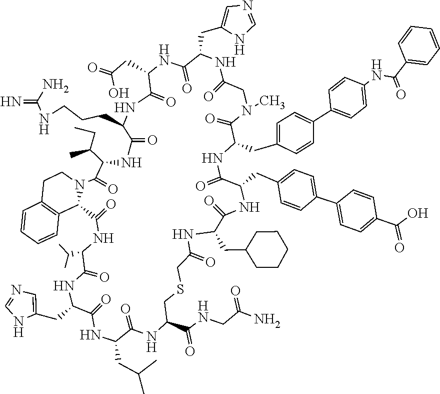 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccccc4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2c3ccccc3CCN2C1=O]

Example 373 was prepared on a 50 umol scale. It was purified by Preparative LC/MS. The yield of the product was 16.2 mg, and its estimated purity by LCMS analysis was 90.5%. Analysis condition 2: Retention time=1.51 min. ESI-MS(+) m/z [M+2H] 2+: 1041.3.

### Preparation of Example 374

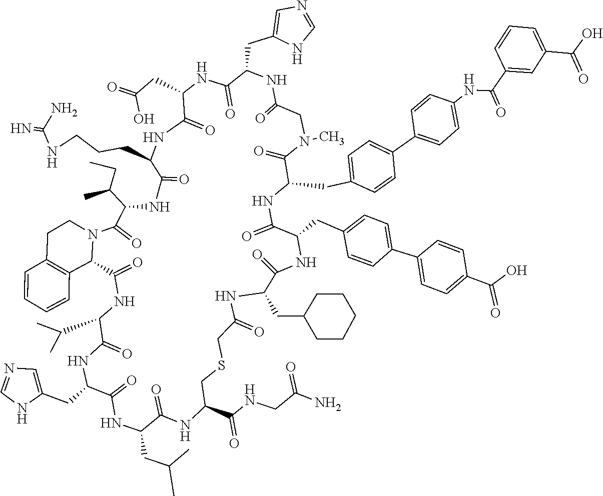 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(C(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2c3ccccc3CCN2C1=O]

Example 374 was prepared on a 50 Preparative LC/MS. The yield of the product was 5.3 mg, and its estimated purity by LCMS analysis was 95.8%. Analysis condition 1: Retention time=1.22 min. ESI-MS(+) m/z [M+2H] 2+: 1063.5.

### Preparation of Example 375

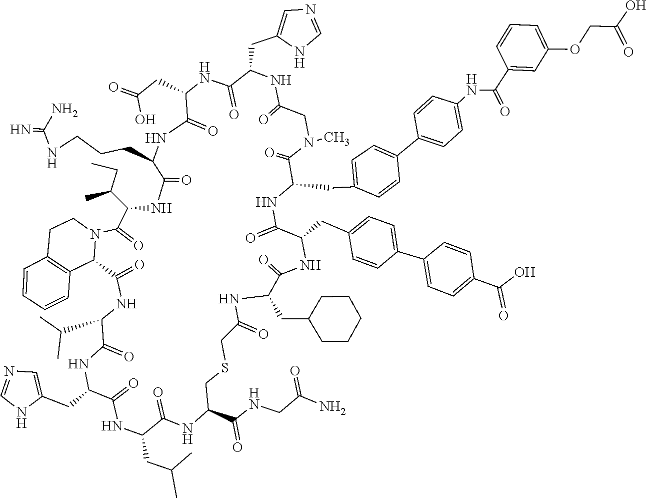 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2c3ccccc3CCN2C1=O]

Example 375 was prepared on a 50 umol scale. It was purified by Preparative LC/MS. The yield of the product was 14.6 mg, and its estimated purity by LCMS analysis was 100%. Analysis condition 2: Retention time=1.34 min. ESI-MS(+) m/z [M+2H] 2+: 1078.8.

### Preparation of Example 376

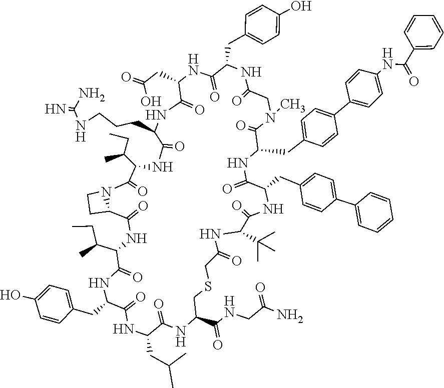 [SMILES: CC[C@H](C)[C@@H]1NC(=O)[C@@H]2CCN2C(=O)[C@H]([C@@H](C)CC)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccc(NC(=O)c4ccccc4)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2ccc(O)cc2)NC1=O]

Example 376 was prepared on a 50 Preparative LC/MS. The yield of the product was 9.3 mg, and its estimated purity by LCMS analysis was 100%. Analysis condition 2: Retention time=1.67 min. ESI-MS(+) m/z [M+H]⁺: 1987.7.

### Preparation of Example 377

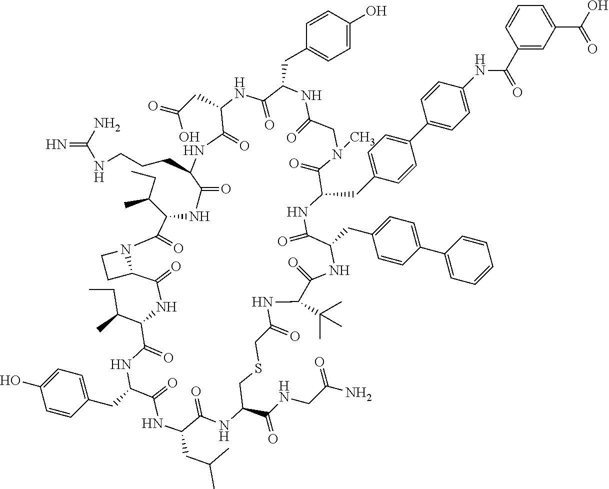 [SMILES: CC[C@H](C)[C@@H]1NC(=O)[C@@H]2CCN2C(=O)[C@H]([C@@H](C)CC)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccc(NC(=O)c4cccc(C(=O)O)c4)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2ccc(O)cc2)NC1=O]

Example 377 was prepared on a 50 umol scale. It was purified by Preparative LC/MS. The yield of the product was 8.4 mg, and its estimated purity by LCMS analysis was 90.8%. Analysis condition 2: Retention time=1.58 min. ESI-MS(+) m/z [M+2H] 2+: 1016.2.

### Preparation of Example 378

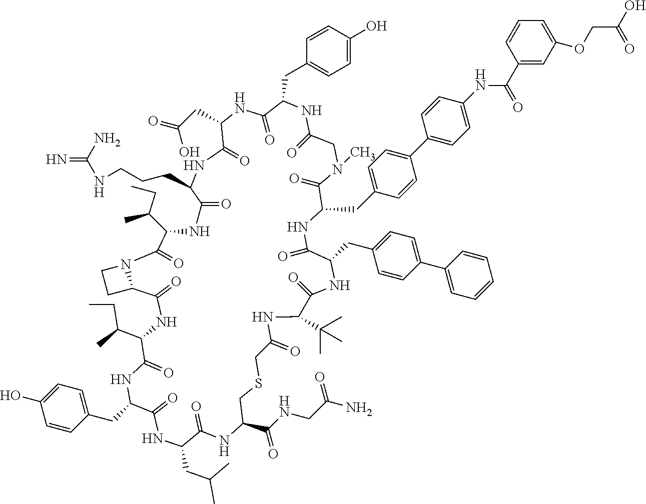 [SMILES: CC[C@H](C)[C@@H]1NC(=O)[C@@H]2CCN2C(=O)[C@H]([C@@H](C)CC)NC(=O)[C@@H](CCCNC(=N)N)NC(=O)[C@H](CC(=O)O)NC(=O)[C@H](Cc2ccc(O)cc2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2ccc(O)cc2)NC1=O]

Example 378 was prepared on a 50 Preparative LC/MS. The yield of the product was 20.3 mg, and its estimated purity by LCMS analysis was 100%. Analysis condition 2: Retention time=1.58 min. ESI-MS(+) m/z [M+2H] 2+: 1031.5.

### Preparation of Example 379

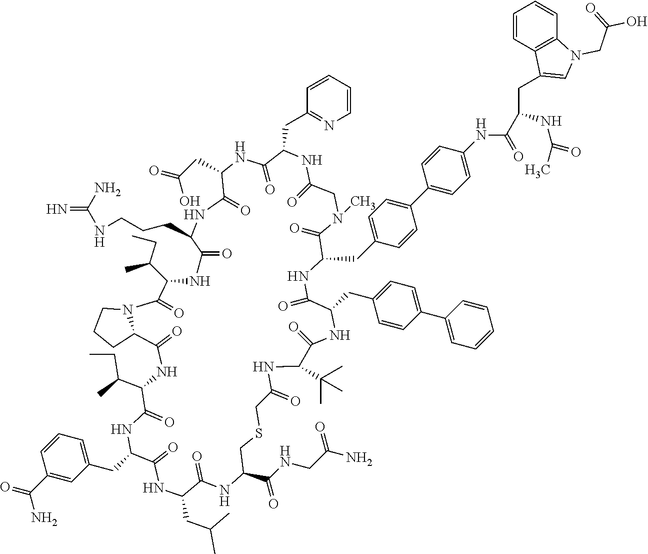 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(=O)[C@H](Cc4cn(CC(=O)O)c5ccccc45)NC(C)=O)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cccc(C(N)=O)c2)NC1=O]

Example 379 was prepared on a 50 umol scale. It was purified by Preparative LC/MS. The yield of the product was 20.3 mg, and its estimated purity by LCMS analysis was 100%. Analysis condition 2: Retention time=1.48 min. ESI-MS(+) m/z [M+2H] 2+: 1098.2.

### Preparation of Example 380

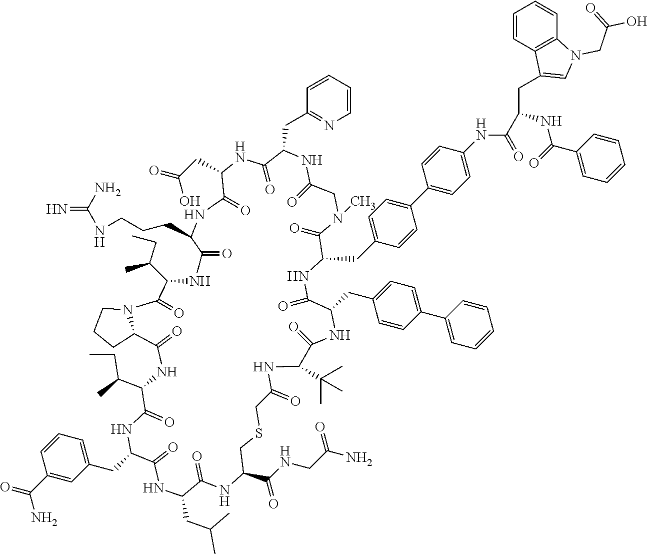 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(=O)[C@H](Cc4cn(CC(=O)O)c5ccccc45)NC(=O)c4ccccc4)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cccc(C(N)=O)c2)NC1=O]

Example 380 was prepared on a 50 Preparative LC/MS. The yield of the product was 2.2 mg, and its estimated purity by LCMS analysis was 97.5%. Analysis condition 1: Retention time=1.52 min. ESI-MS(+) m/z [M+2H] 2+: 1129.5.

### Preparation of Example 381

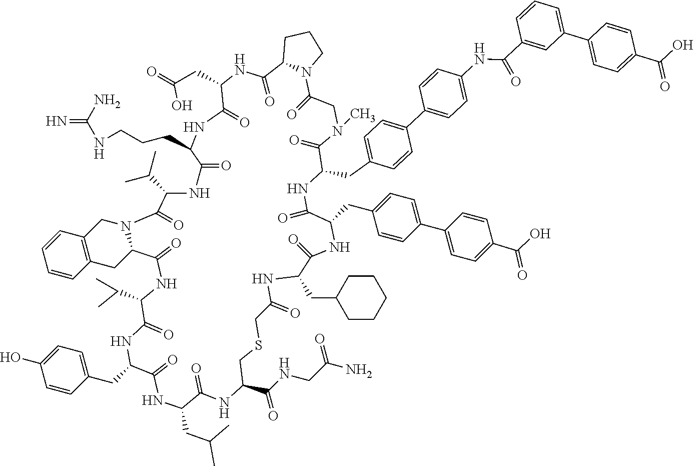 [SMILES: CC(C)C[C@@H]1NC(=O)[C@H](Cc2ccc(O)cc2)NC(=O)[C@H](C(C)C)NC(=O)[C@@H]2Cc3ccccc3CN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4cccc(-c5ccc(C(=O)O)cc5)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 381 was prepared on a 50 umol scale. It was purified by Preparative LC/MS. The yield of the product was 7.7 mg, and its estimated purity by LCMS analysis was 100%. Analysis condition 2: Retention time=1.63 min. ESI-MS(+) m/z [M+2H] 2+: 1087.8.

### Preparation of Example 382

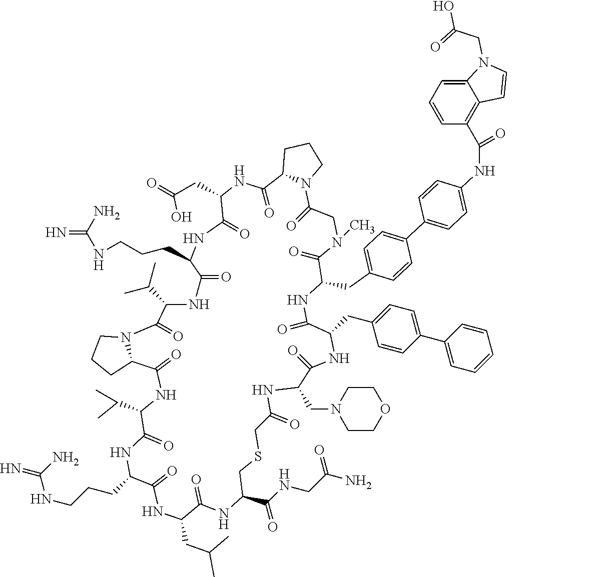 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4cccc5c4ccn5CC(=O)O)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 382 was prepared on a 50 Preparative LC/MS. The yield of the product was 4.87 mg, and its estimated purity by LCMS analysis was 93%. Analysis condition 1: Retention time=0.71 min. ESI-MS(+) m/z [M+2H] 2+: 1020.6.

### Preparation of Example 383

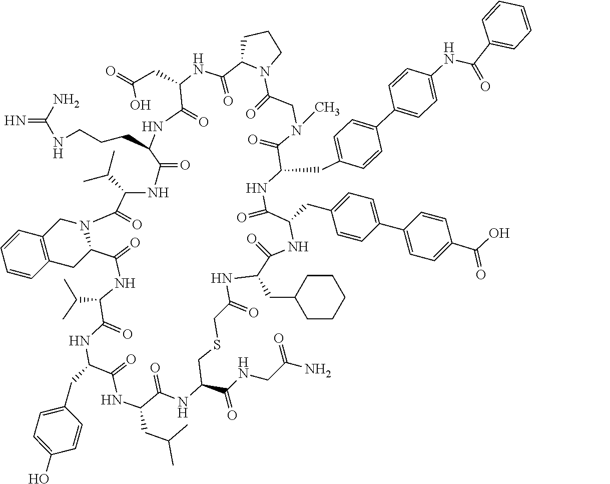 [SMILES: CC(C)C[C@@H]1NC(=O)[C@H](Cc2ccc(O)cc2)NC(=O)[C@H](C(C)C)NC(=O)[C@@H]2Cc3ccccc3CN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4ccccc4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 383 was prepared on a 50 umol scale. It was purified by Preparative LC/MS. The yield of the product was 9.4 mg, and its estimated purity by LCMS analysis was 95.1%. Analysis condition 1: Retention time=1.49 min. ESI-MS(+) m/z [M+2H] 2+: 1027.4.

### Preparation of Example 384

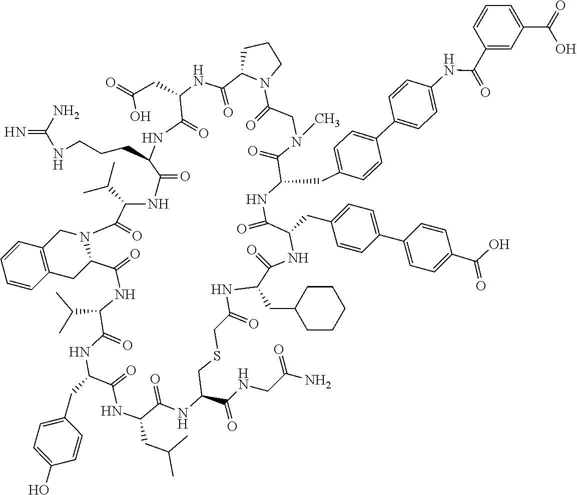 [SMILES: CC(C)C[C@@H]1NC(=O)[C@H](Cc2ccc(O)cc2)NC(=O)[C@H](C(C)C)NC(=O)[C@@H]2Cc3ccccc3CN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4cccc(C(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 384 was prepared on a 50 Preparative LC/MS. The yield of the product was 10.4 mg, and its estimated purity by LCMS analysis was 100%. Analysis condition 1: Retention time=1.2 min. ESI-MS(+) m/z [M+2H] 2+: 1049.6.

### Preparation of Example 385

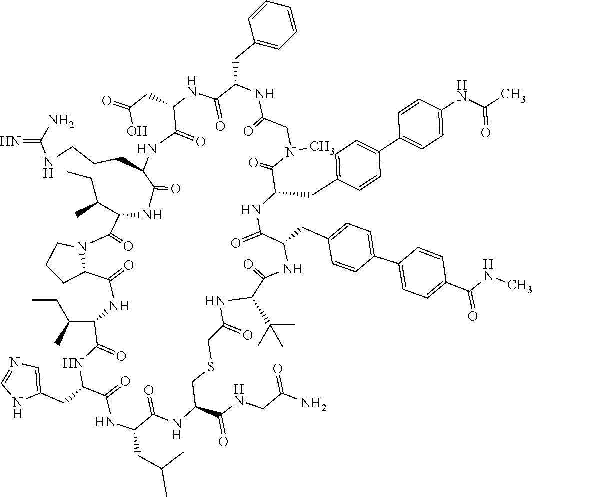 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccc2)NC(=O)CN(C)C(=O)[C@H](Cc2ccc(-c3ccc(NC(C)=O)cc3)cc2)NC(=O)[C@H](Cc2ccc(-c3ccc(C(=O)NC)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cnc[nH]2)NC1=O]

Example 385 was prepared on a 50 umol scale. It was purified by Preparative LC/MS. The yield of the product was 15.5 mg, and its estimated purity by LCMS analysis was 99.1%. Analysis condition 1: Retention time=1.37 min. ESI-MS(+) m/z [M+H]⁺: 1953.8.

### Preparation of Example 386

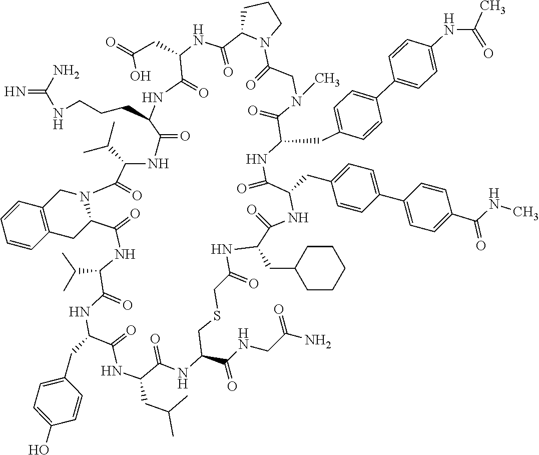 [SMILES: CNC(=O)c1ccc(-c2ccc(C[C@@H]3NC(=O)[C@H](CC4CCCCC4)NC(=O)CSC[C@@H](C(=O)NCC(N)=O)NC(=O)[C@H](CC(C)C)NC(=O)[C@H](Cc4ccc(O)cc4)NC(=O)[C@H](C(C)C)NC(=O)[C@@H]4Cc5ccccc5CN4C(=O)[C@H](C(C)C)NC(=O)[C@@H](CCCNC(=N)N)NC(=O)[C@H](CC(=O)O)NC(=O)[C@@H]4CCCN4C(=O)CN(C)C(=O)[C@H](Cc4ccc(-c5ccc(NC(C)=O)cc5)cc4)NC3=O)cc2)cc1]

Example 386 was prepared on a 50 Preparative LC/MS. The yield of the product was 8.6 mg, and its estimated purity by LCMS analysis was 100%. Analysis condition 1: Retention time=1.38 min. ESI-MS(+) m/z [M+2H] 2+: 1003.

### Preparation of Example 387

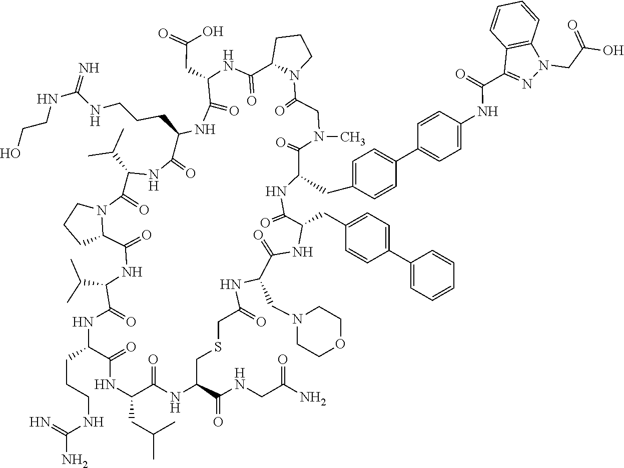 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCO)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 387 was prepared on a 100 umol scale. It was purified by Preparative LC/MS. The yield of the product was 12.5 mg, and its estimated purity by LCMS analysis was 100%. Analysis condition 1: Retention time=1.41 min. ESI-MS(+) m/z [M+2H] 2+: 1043.4

### Preparation of Example 388

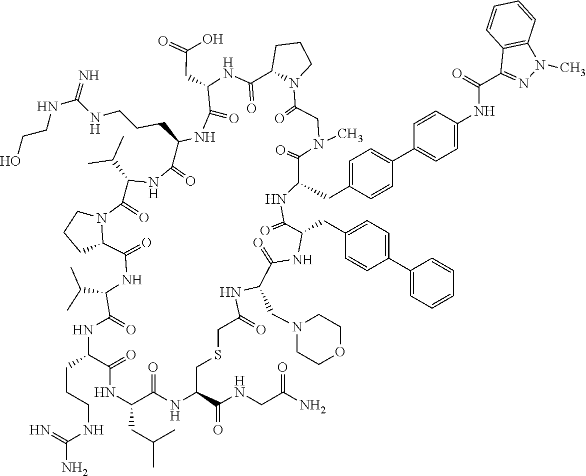 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCO)NC(=O)[C@H](CC(=O)O)NC(=O)[C@@H]2CCCN2C(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 388 was prepared on a 100 Preparative LC/MS. The yield of the product was 5.3 mg, and its estimated purity by LCMS analysis was 100%. Analysis condition 1: Retention time=1.55 min. ESI-MS(+) m/z [M+2H] 2+: 1021.5.

### Preparation of Example 389

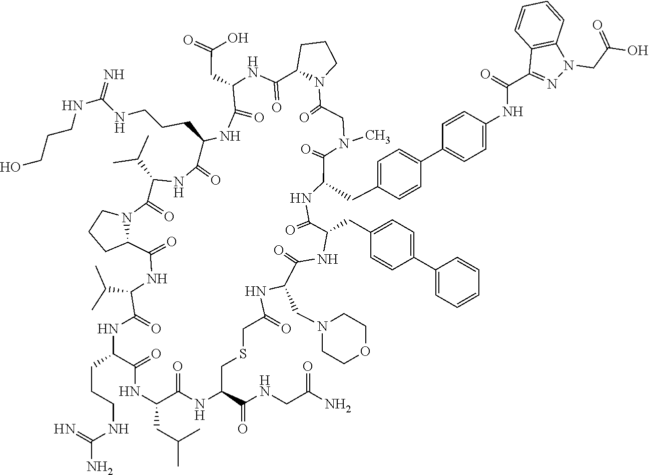 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCCO)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 389 was prepared on a 100 umol scale. It was purified by Preparative LC/MS. The yield of the product was 9.3 mg, and its estimated purity by LCMS analysis was 100%. Analysis condition 2: Retention time=1.35 min. ESI-MS(+) m/z [M+2H] 2+: 1050.8.

### Preparation of Example 390

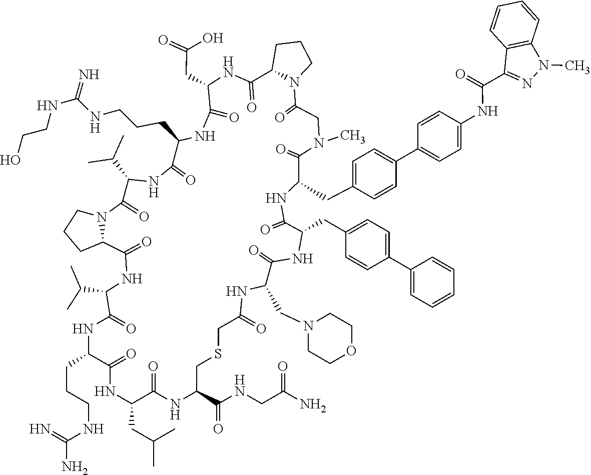 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCO)NC(=O)[C@H](CC(=O)O)NC(=O)[C@@H]2CCCN2C(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 390 was prepared on a 100 Preparative LC/MS. The yield of the product was 19.3 mg, and its estimated purity by LCMS analysis was 100%. Analysis condition 1: Retention time=1.49 min. ESI-MS(+) m/z [M+2H] 2+: 1028.5.

### Preparation of Example 391

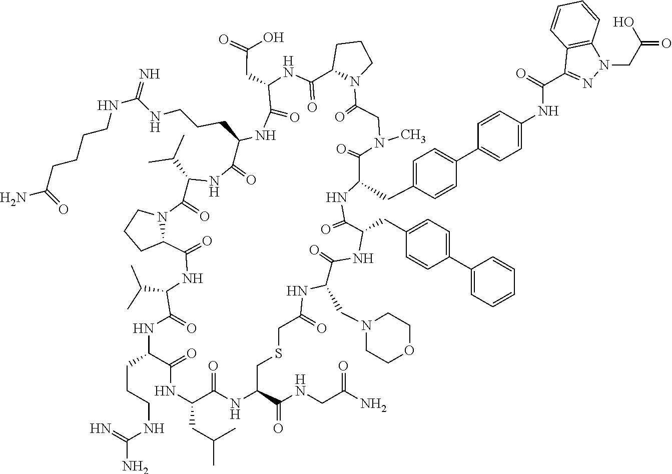 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCCCC(N)=O)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 391 was prepared on a 100 umol scale. It was purified by Preparative LC/MS. The yield of the product was 11.3 mg, and its estimated purity by LCMS analysis was 100%. Analysis condition 1: Retention time=1.3 min. ESI-MS(+) m/z [M+2H] 2+: 1070.9.

### Preparation of Example 392

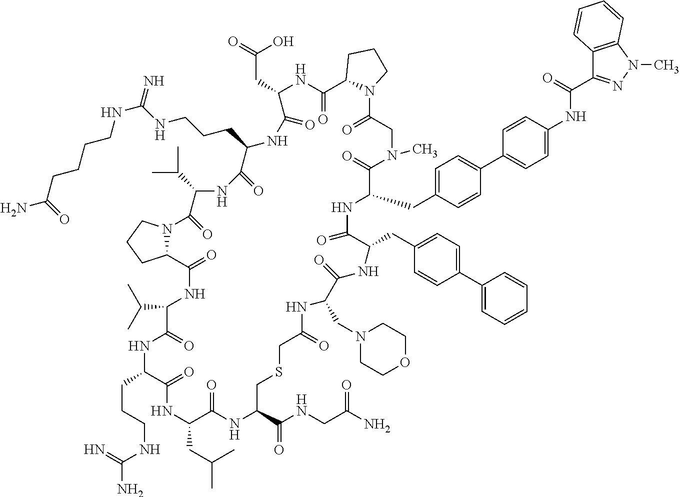 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCCCC(N)=O)NC(=O)[C@H](CC(=O)O)NC(=O)[C@@H]2CCCN2C(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 392 was prepared on a 100 Preparative LC/MS. The yield of the product was 11.4 mg, and its estimated purity by LCMS analysis was 91.9%. Analysis condition 1: Retention time=1.46 min. ESI-MS(+) m/z [M+2H] 2+: 1048.9.

### Preparation of Example 393

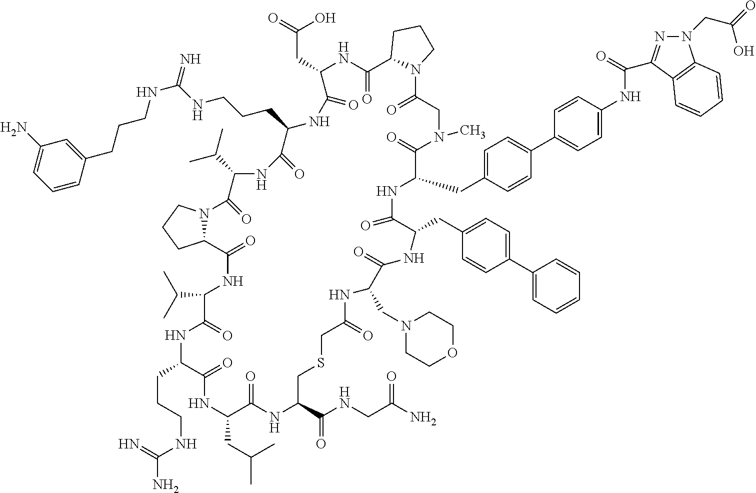 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCCc2cccc(N)c2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 393 was prepared on a 100 umol scale. It was purified by Preparative LC/MS. The yield of the product was 4.6 mg, and its estimated purity by LCMS analysis was 95.8%. Analysis condition 1: Retention time=1.45 min. ESI-MS(+) m/z [M+2H] 2+: 1088.

### Preparation of Example 394

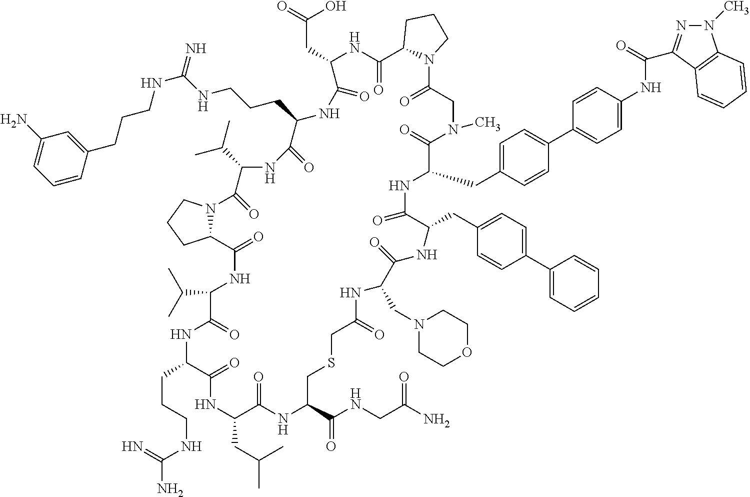 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCCc2cccc(N)c2)NC(=O)[C@H](CC(=O)O)NC(=O)[C@@H]2CCCN2C(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 394 was prepared on a 100 Preparative LC/MS. The yield of the product was 5 mg, and its estimated purity by LCMS analysis was 97.9%. Analysis condition 2: Retention time=1.44 min. ESI-MS(+) m/z [M+3H] 3+: 711.1.

### Preparation of Example 395

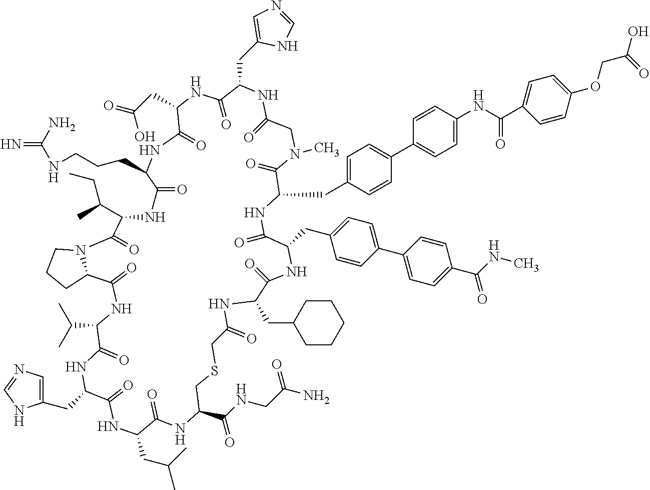 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccc(OCC(=O)O)cc4)cc3)cc2)NC(=O)[C@H](Cc2ccc(-c3ccc(C(=O)NC)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 395 was prepared on a 50 umol scale. It was purified by Preparative LC/MS. The yield of the product was 10.9 mg, and its estimated purity by LCMS analysis was 96.9%. Analysis condition 2: Retention time=1.29 min. ESI-MS(+) m/z [M+2H] 2+: 1054.1.

### Preparation of Example 396

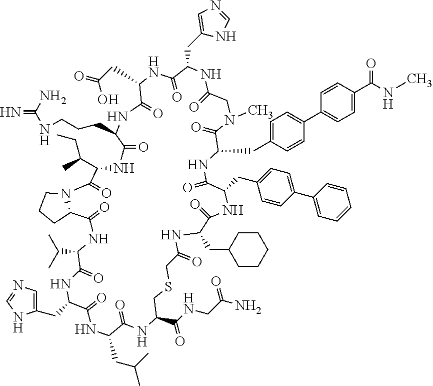 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(C(=O)NC)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 396 was prepared on a 50 Preparative LC/MS. The yield of the product was 42.2 mg, and its estimated purity by LCMS analysis was 97.5%. Analysis condition 2: Retention time=1.33 min. ESI-MS(+) m/z [M+H]⁺: 1912.7.

### Preparation of Example 397

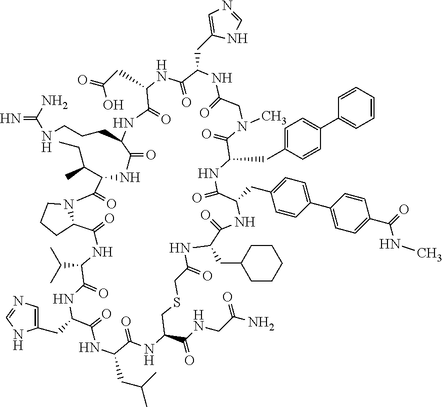 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccccc3)cc2)NC(=O)[C@H](Cc2ccc(-c3ccc(C(=O)NC)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 397 was prepared on a 50 umol scale. It was purified by Preparative LC/MS. The yield of the product was 18.1 mg, and its estimated purity by LCMS analysis was 97.5%. Analysis condition 1: Retention time=1.5 min. ESI-MS(+) m/z [M+H]⁺: 1912.8.

### Preparation of Example 398

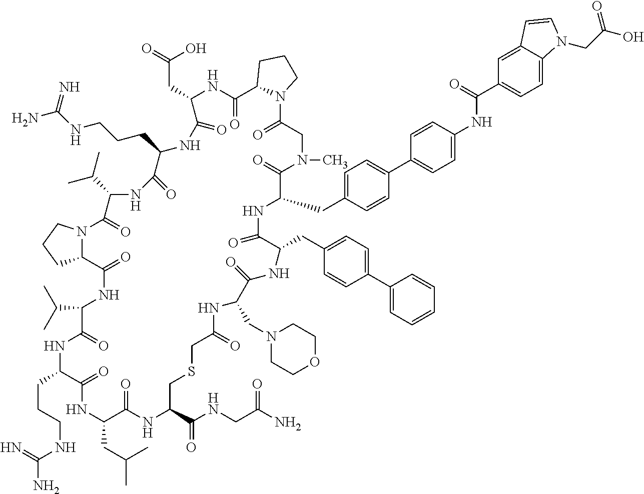 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4ccc5c(ccn5CC(=O)O)c4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 398 was prepared on a 50 umol scale. It was purified by Preparative LC/MS. The yield of the product was 1.4 mg, and its estimated purity by LCMS analysis was 99%. Analysis condition 2: Retention time=1.3 min. ESI-MS(+) m/z [M+2H] 2+: 1020.8.

### Preparation of Example 399

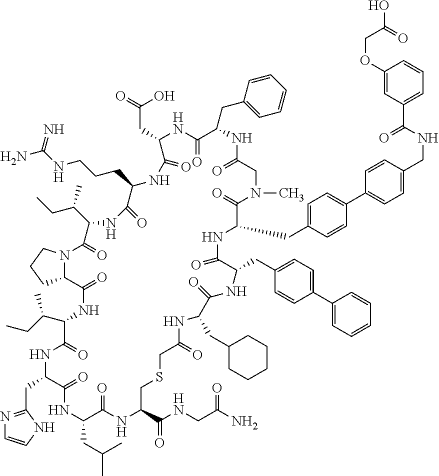 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccc2)NC(=O)CN(C)C(=O)[C@H](Cc2ccc(-c3ccc(CNC(=O)c4cccc(OCC(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2ncc[nH]2)NC1=O]

Example 399 was prepared on a 50 umol scale. It was purified by Preparative LC/MS. The yield of the product was 38.5 mg, and its estimated purity by LCMS analysis was 94.8%. Analysis condition 1: Retention time=1.6 min. ESI-MS(+) m/z [M+2H] 2+: 1044.5.

### Preparation of Example 400

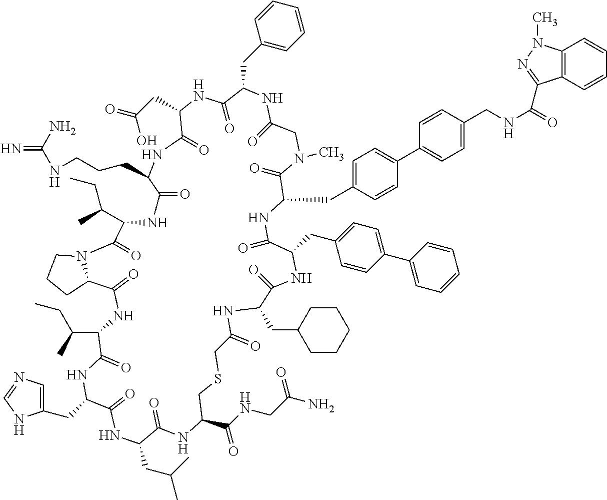 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccc2)NC(=O)CN(C)C(=O)[C@H](Cc2ccc(-c3ccc(CNC(=O)c4nn(C)c5ccccc45)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC1=O]

Example 400 was prepared on a 50 Preparative LC/MS. The yield of the product was 8.5 mg, and its estimated purity by LCMS analysis was 99.3%. Analysis condition 2: Retention time=1.83 min. ESI-MS(+) m/z [M+2H] 2+: 1034.5.

### Preparation of Example 401

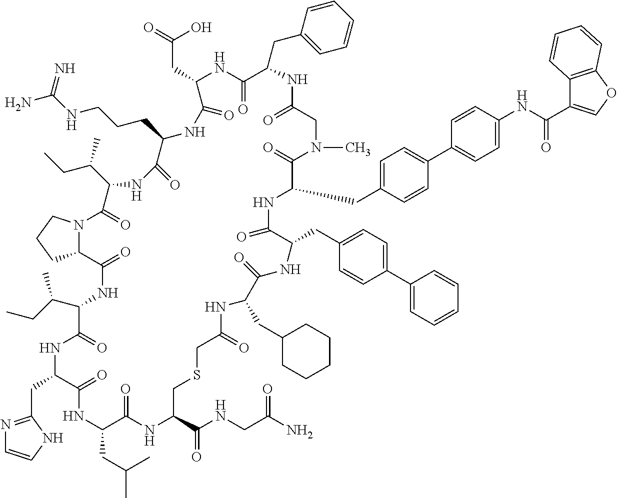 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccc2)NC(=O)CN(C)C(=O)[C@H](Cc2ccc(-c3ccc(NC(=O)c4coc5ccccc45)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2ncc[nH]2)NC1=O]

Example 401 was prepared on a 50 umol scale. It was purified by Preparative LC/MS. The yield of the product was 20.3 mg, and its estimated purity by LCMS analysis was 95.4%. Analysis condition 2: Retention time=1.96 min. ESI-MS(+) m/z [M+2H] 2+: 1020.4.

### Preparation of Example 402

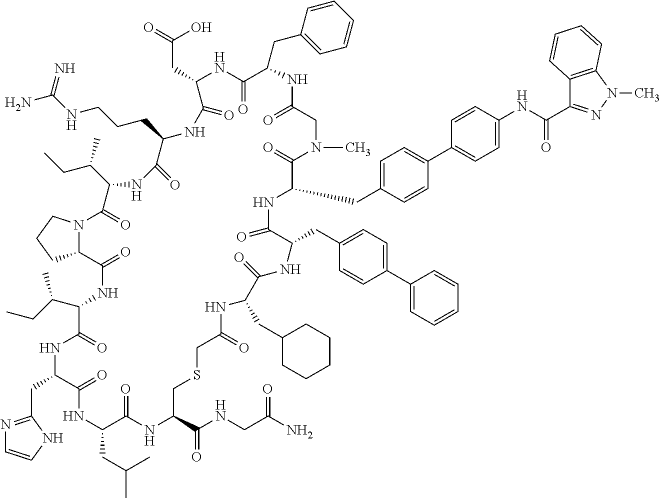 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccc2)NC(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2ncc[nH]2)NC1=O]

Example 402 was prepared on a 50 Preparative LC/MS. The yield of the product was 31.8 mg, and its estimated purity by LCMS analysis was 100%. Analysis condition 1: Retention time=1.81 min. ESI-MS(+) m/z [M+2H] 2+: 1027.3.

### Preparation of Example 403

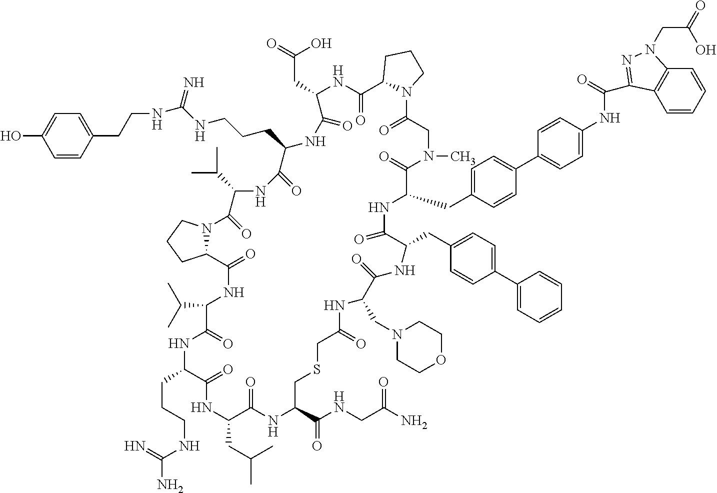 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCc2ccc(O)cc2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 403 was prepared on a 100 umol scale. It was purified by Preparative LC/MS. The yield of the product was 13.3 mg, and its estimated purity by LCMS analysis was 88.6%. Analysis condition 2: Retention time=1.53 min. ESI-MS(+) m/z [M+2H] 2+: 1081.6.

### Preparation of Example 404

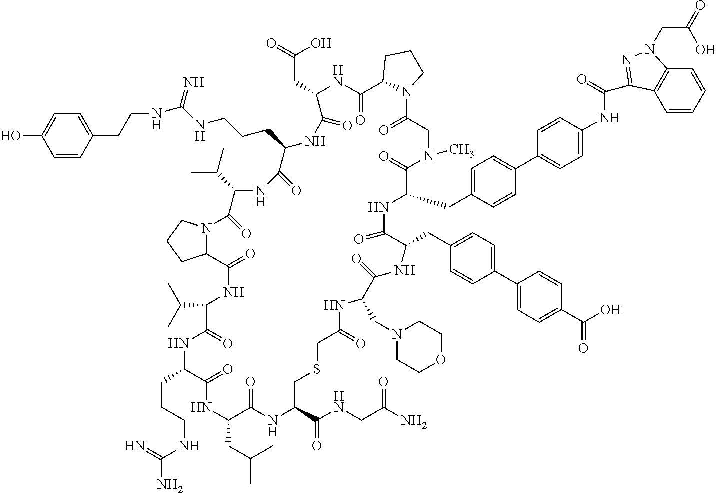 [SMILES: CC(C)C[C@@H]1NC(=O)[C@H](CCCNC(=N)N)NC(=O)[C@H](C(C)C)NC(=O)C2CCCN2C(=O)[C@H](C(C)C)NC(=O)[C@@H](CCCNC(=N)NCCc2ccc(O)cc2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC1=O]

Example 404 was prepared on a 100 Preparative LC/MS. The yield of the product was 1.5 mg, and its estimated purity by LCMS analysis was 96.8%. Analysis condition 2: Retention time=1.39 min. ESI-MS(+) m/z [M+2H] 2+: 1103.4.

### Preparation of Example 405

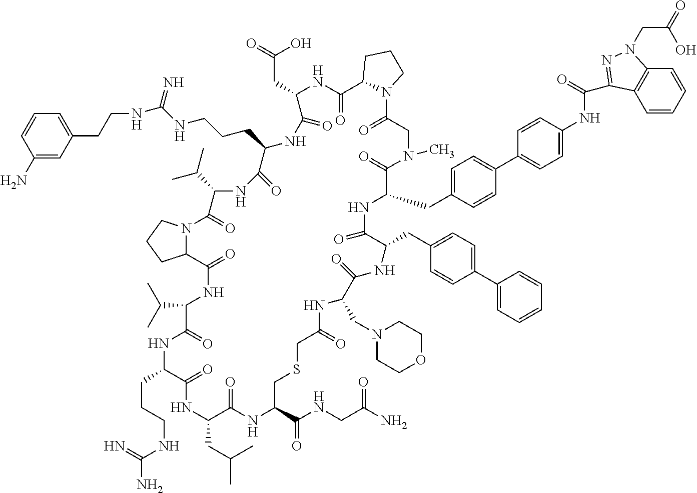 [SMILES: CC(C)C[C@@H]1NC(=O)[C@H](CCCNC(=N)N)NC(=O)[C@H](C(C)C)NC(=O)C2CCCN2C(=O)[C@H](C(C)C)NC(=O)[C@@H](CCCNC(=N)NCCc2cccc(N)c2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 405 was prepared on a 100 umol scale. It was purified by Preparative LC/MS. The yield of the product was 6.7 mg, and its estimated purity by LCMS analysis was 87.2%. Analysis condition 2: Retention time=1.43 min. ESI-MS(+) m/z [M+2H] 2+: 1081.

### Preparation of Example 406

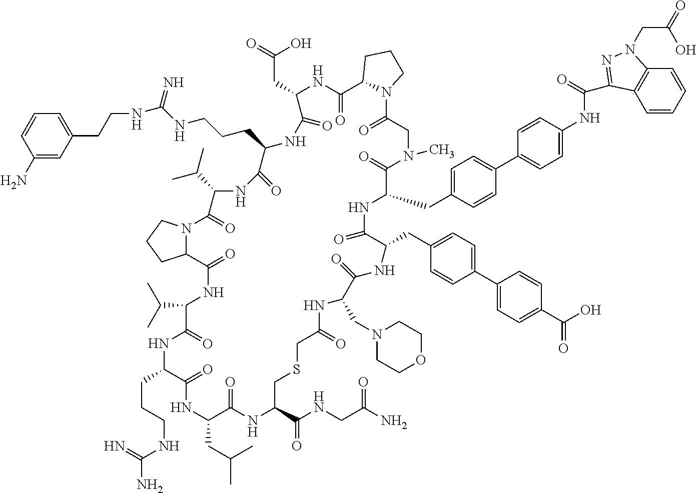 [SMILES: CC(C)C[C@@H]1NC(=O)[C@H](CCCNC(=N)N)NC(=O)[C@H](C(C)C)NC(=O)C2CCCN2C(=O)[C@H](C(C)C)NC(=O)[C@@H](CCCNC(=N)NCCc2cccc(N)c2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC1=O]

Example 406 was prepared on a 100 Preparative LC/MS. The yield of the product was 3.1 mg, and its estimated purity by LCMS analysis was 91.9%. Analysis condition 1: Retention time=1.12 min. ESI-MS(+) m/z [M+2H] 2+: 1102.9.

### Preparation of Example 407

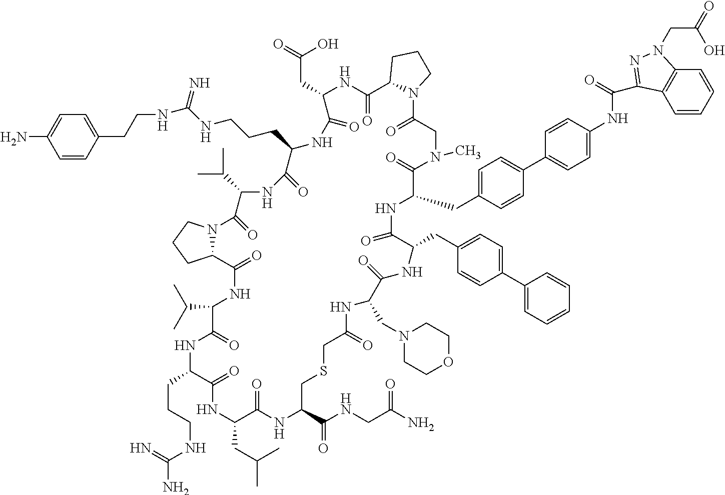 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCc2ccc(N)cc2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 407 was prepared on a 100 umol scale. It was purified by Preparative LC/MS. The yield of the product was 6.7 mg, and its estimated purity by LCMS analysis was 98.3%. Analysis condition 2: Retention time=1.41 min. ESI-MS(+) m/z [M+2H] 2+: 1081.

### Preparation of Example 408

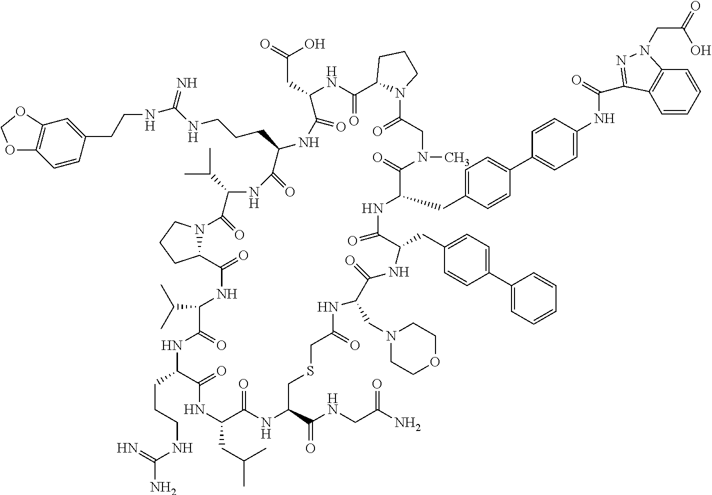 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCc2ccc3c(c2)OCO3)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 408 was prepared on a 100 Preparative LC/MS. The yield of the product was 7.6 mg, and its estimated purity by LCMS analysis was 90.7%. Analysis condition 2: Retention time=1.61 min. ESI-MS(+) m/z [M+2H] 2+: 1095.4.

### Preparation of Example 409

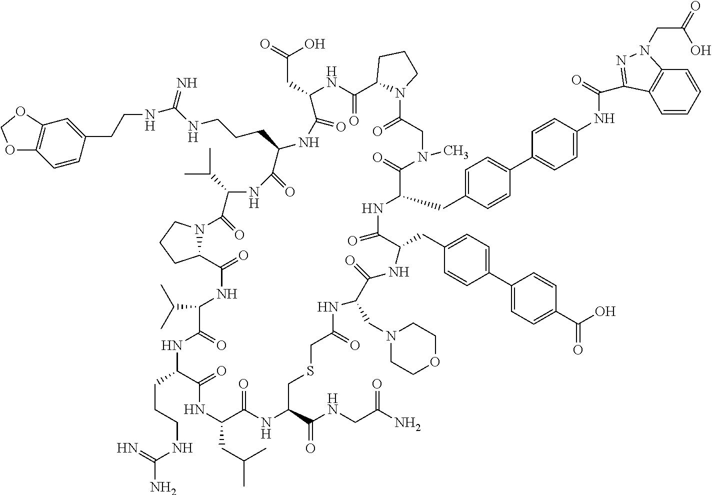 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCc2ccc3c(c2)OCO3)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC1=O]

Example 409 was prepared on a 100 umol scale. It was purified by Preparative LC/MS. The yield of the product was 3.1 mg, and its estimated purity by LCMS analysis was 95.3%. Analysis condition 2: Retention time=1.54 min. ESI-MS(+) m/z [M+2H] 2+: 1117.6.

### Preparation of Example 410

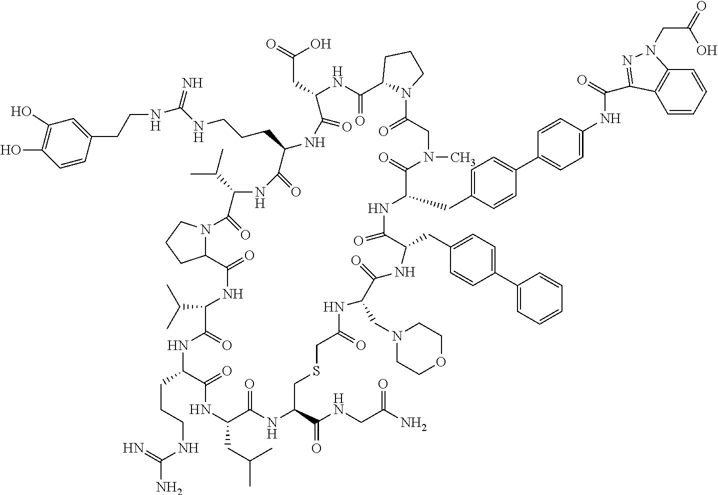 [SMILES: CC(C)C[C@@H]1NC(=O)[C@H](CCCNC(=N)N)NC(=O)[C@H](C(C)C)NC(=O)C2CCCN2C(=O)[C@H](C(C)C)NC(=O)[C@@H](CCCNC(=N)NCCc2ccc(O)c(O)c2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 410 was prepared on a 1.827 umol scale. It was purified by Preparative LC/MS. The yield of the product was 1.9 mg, and its estimated purity by LCMS analysis was 100%. Analysis condition 2: Retention time=1.38 min. ESI-MS(+) m/z [M+2H] 2+: 1089.4.

### Preparation of Example 411

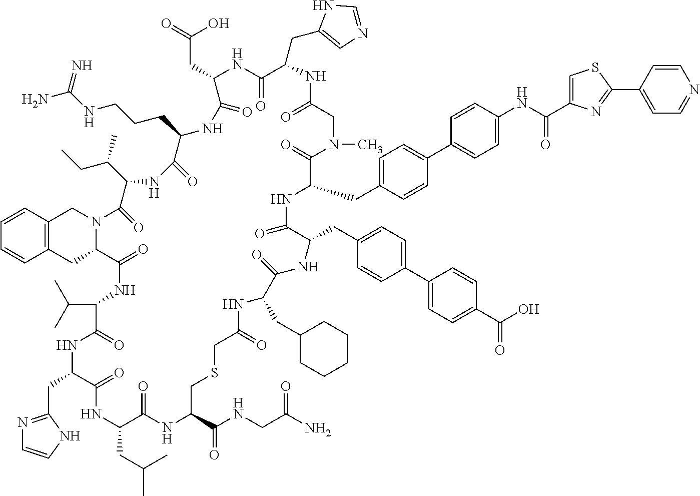 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4csc(-c5ccncc5)n4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2ncc[nH]2)NC(=O)[C@H](C(C)C)NC(=O)[C@@H]2Cc3ccccc3CN2C1=O]

Example 411 was prepared on a 50 umol scale. It was purified by Preparative LC/MS. The yield of the product was 1.3 mg, and its estimated purity by LCMS analysis was 94.9%. Analysis condition 1: Retention time=1.37 min. ESI-MS(+) m/z [M+3H] 3+: 722.6.

### Preparation of Example 412

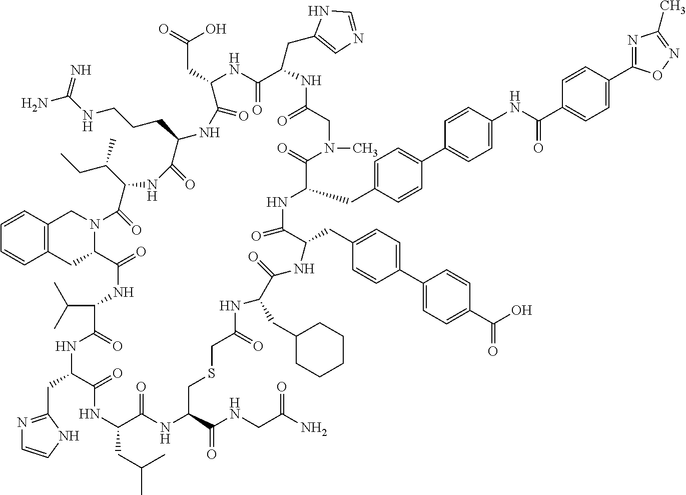 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccc(-c5nc(C)no5)cc4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2ncc[nH]2)NC(=O)[C@H](C(C)C)NC(=O)[C@@H]2Cc3ccccc3CN2C1=O]

Example 412 was prepared on a 50 Preparative LC/MS. The yield of the product was 1.5 mg, and its estimated purity by LCMS analysis was 95.3%. Analysis condition 2: Retention time=1.54 min. ESI-MS(+) m/z [M+2H] 2+: 1082.5.

### Preparation of Example 413

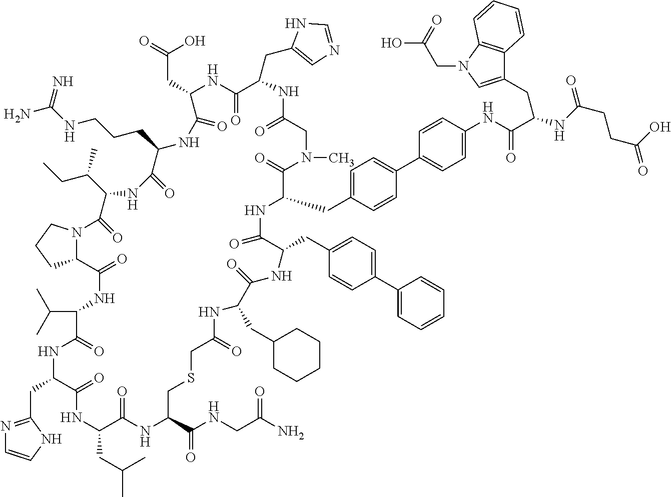 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)[C@H](Cc4cn(CC(=O)O)c5ccccc45)NC(=O)CCC(=O)O)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2ncc[nH]2)NC(=O)[C@H](C(C)C)NC(=O)[C@@H]2CCCN2C1=O]

Example 413 was prepared on a 50 umol scale. It was purified by Preparative LC/MS. The yield of the product was 2.9 mg, and its estimated purity by LCMS analysis was 97.5%. Analysis condition 1: Retention time=1.42 min. ESI-MS(+) m/z [M+2H] 2+: 1108.1.

### Preparation of Example 414

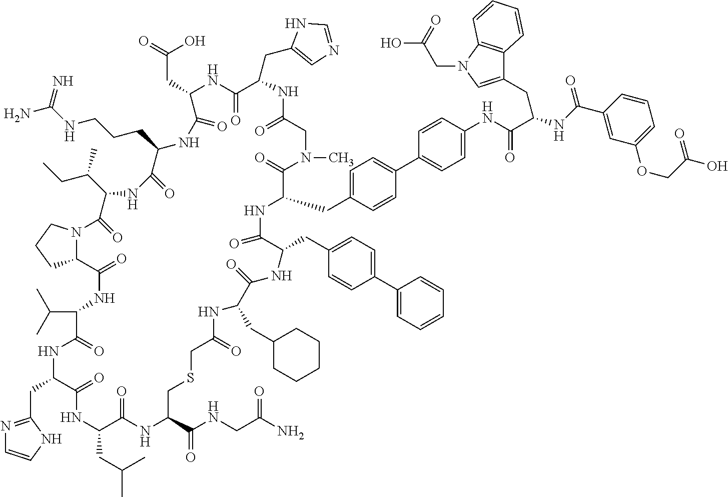 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)[C@H](Cc4cn(CC(=O)O)c5ccccc45)NC(=O)c4cccc(OCC(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2ncc[nH]2)NC(=O)[C@H](C(C)C)NC(=O)[C@@H]2CCCN2C1=O]

Example 414 was prepared on a 50 Preparative LC/MS. The yield of the product was 5.4 mg, and its estimated purity by LCMS analysis was 95.3%. Analysis condition 1: Retention time=1.48 min. ESI-MS(+) m/z [M+2H] 2+: 1147.7.

### Preparation of Example 415

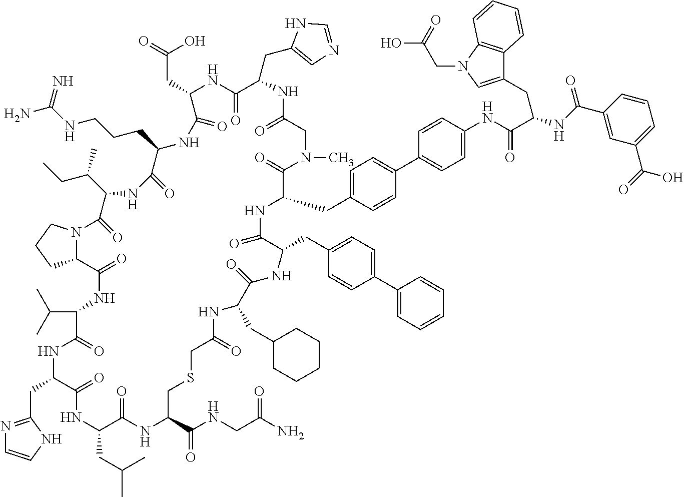 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)[C@H](Cc4cn(CC(=O)O)c5ccccc45)NC(=O)c4cccc(C(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2ncc[nH]2)NC(=O)[C@H](C(C)C)NC(=O)[C@@H]2CCCN2C1=O]

Example 415 was prepared on a 50 umol scale. It was purified by Preparative LC/MS. The yield of the product was 5.3 mg, and its estimated purity by LCMS analysis was 98.3%. Analysis condition 1: Retention time=1.44 min. ESI-MS(+) m/z [M+2H] 2+: 1132.6.

### Preparation of Example 416

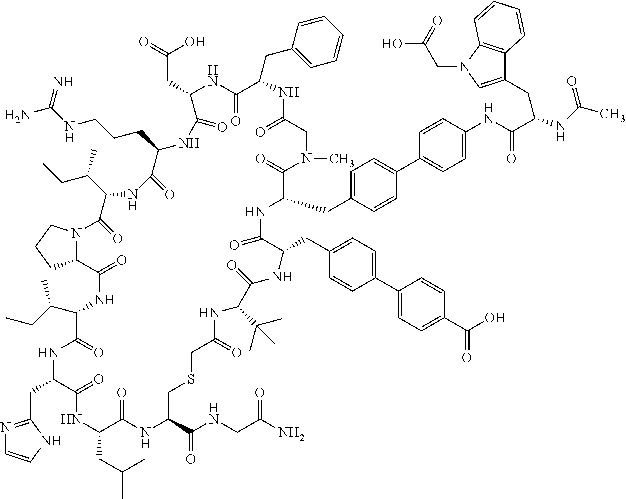 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccc2)NC(=O)CN(C)C(=O)[C@H](Cc2ccc(-c3ccc(NC(=O)[C@H](Cc4cn(CC(=O)O)c5ccccc45)NC(C)=O)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2ncc[nH]2)NC1=O]

Example 416 was prepared on a 50 Preparative LC/MS. The yield of the product was 2.7 mg, and its estimated purity by LCMS analysis was 99.2%. Analysis condition 1: Retention time=1.47 min. ESI-MS(+) m/z [M+2H] 2+: 1092.9.

### Preparation of Example 417

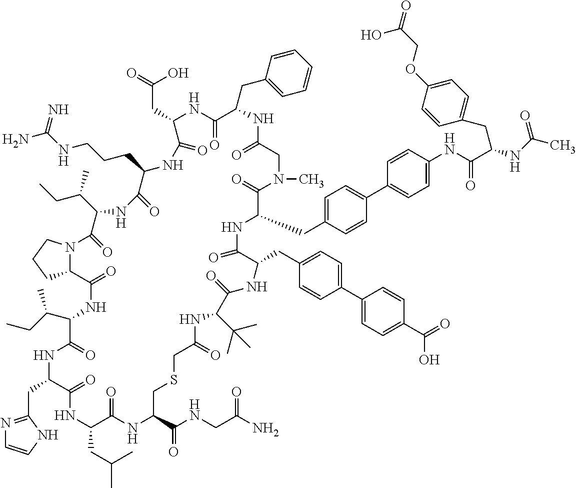 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccc2)NC(=O)CN(C)C(=O)[C@H](Cc2ccc(-c3ccc(NC(=O)[C@H](Cc4ccc(OCC(=O)O)cc4)NC(C)=O)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2ncc[nH]2)NC1=O]

Example 417 was prepared on a 50 umol scale. It was purified by Preparative LC/MS. The yield of the product was 4.6 mg, and its estimated purity by LCMS analysis was 96.6%. Analysis condition 1: Retention time=1.45 min. ESI-MS(+) m/z [M+2H] 2+: 1082.0.

### Preparation of Example 418

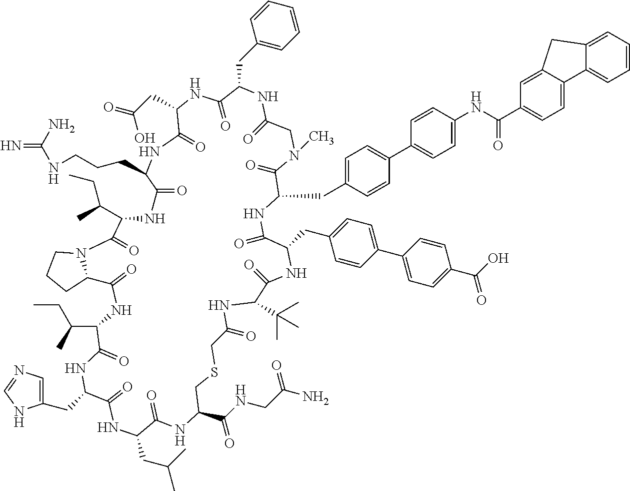 [SMILES: CC[C@H](C)[C@@H]1NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2ccccc2)NC(=O)CN(C)C(=O)[C@H](Cc2ccc(-c3ccc(NC(=O)c4ccc5c(c4)Cc4ccccc4-5)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cnc[nH]2)NC1=O]

Example 418 was prepared on a 50 Preparative LC/MS. The yield of the product was 16.4 mg, and its estimated purity by LCMS analysis was 95.7%. Analysis condition 1: Retention time=1.64 min. ESI-MS(+) m/z [M+2H] 2+: 1046.4.

### Preparation of Example 419

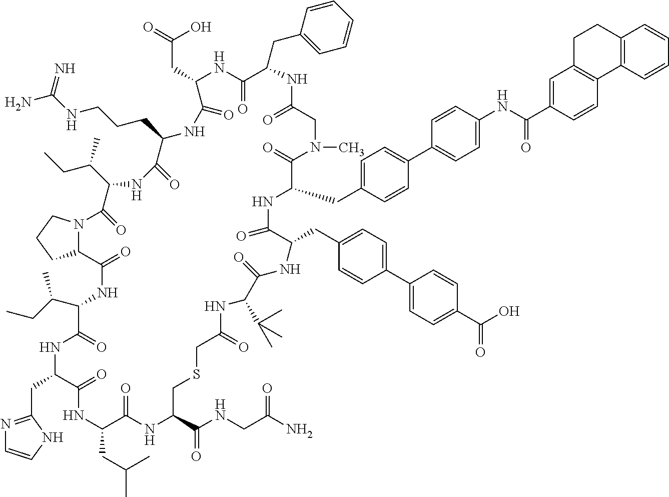

Example 419 was prepared on a 50 umol scale. It was purified by Preparative LC/MS. The yield of the product was 25.9 mg, and its estimated purity by LCMS analysis was 91%. Analysis condition 1: Retention time=1.71 min. ESI-MS(+) m/z [M+2H] 2+: 1053.2.

### Preparation of Example 420

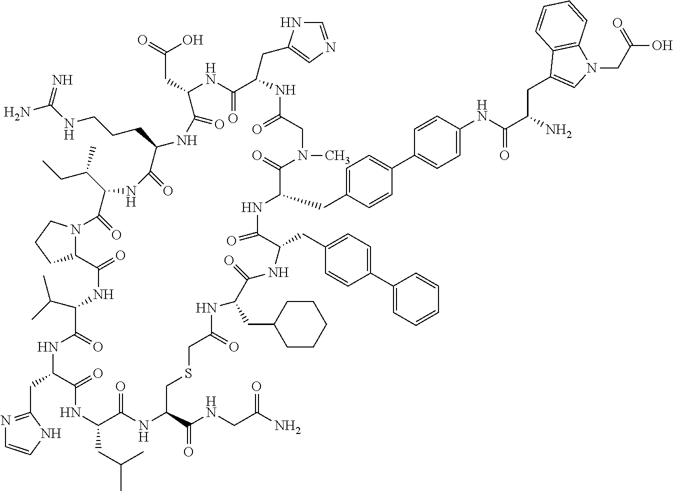

Example 420 was prepared on a 50 Preparative LC/MS. The yield of the product was 28.1 mg, and its estimated purity by LCMS analysis was 97.2%. Analysis condition 2: Retention time=1.33 min. ESI-MS(+) m/z [M+2H] 2+: 1058.4.

### Preparation of Example 421

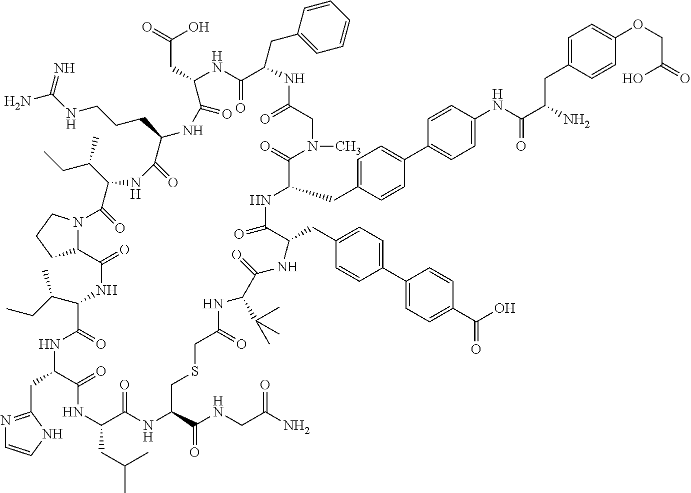

Example 421 was prepared on a 50 umol scale. It was purified by Preparative LC/MS. The yield of the product was 11 mg, and its estimated purity by LCMS analysis was 99%. Analysis condition 2: Retention time 1.38 min. ESI-MS(+) m/z [M+2H] 2+: 1061.

### Preparation of Example 422

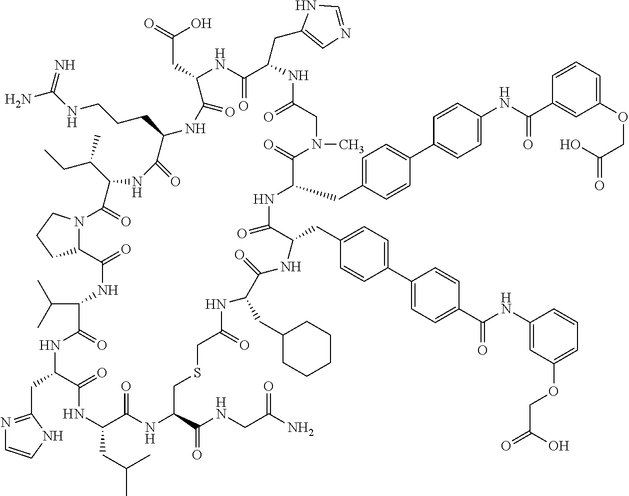

Example 422 was prepared on a 50 Preparative LC/MS. The yield of the product was 7 mg, and its estimated purity by LCMS analysis was 98.1%. Analysis condition 2: Retention time=1.35 min. ESI-MS(+) m/z [M+2H] 2+: 1122.1.

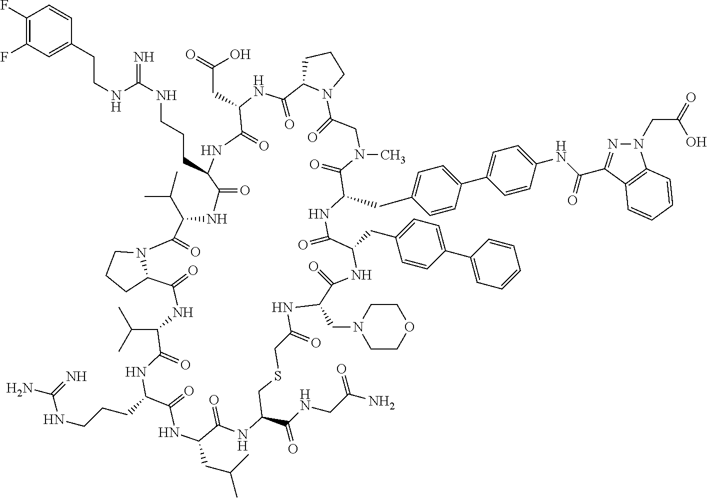 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCc2ccc(F)c(F)c2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

### Step 1: Synthesis of the Linear Sequence on Symphony Peptide Synthesizer

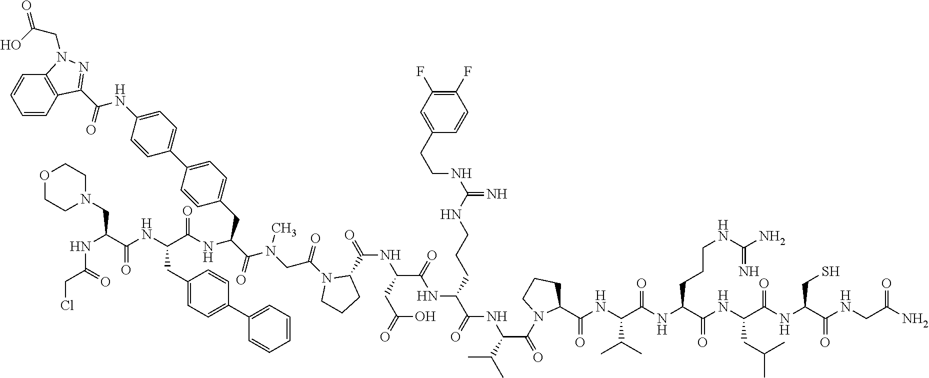 [SMILES: CC(C)C[C@H](NC(=O)[C@H](CCCNC(=N)N)NC(=O)[C@@H](NC(=O)[C@@H]1CCCN1C(=O)[C@@H](NC(=O)[C@@H](CCCNC(=N)NCCc1ccc(F)c(F)c1)NC(=O)[C@H](CC(=O)O)NC(=O)[C@@H]1CCCN1C(=O)CN(C)C(=O)[C@H](Cc1ccc(-c2ccc(NC(=O)c3nn(CC(=O)O)c4ccccc34)cc2)cc1)NC(=O)[C@H](Cc1ccc(-c2ccccc2)cc1)NC(=O)[C@H](CN1CCOCC1)NC(=O)CCl)C(C)C)C(C)C)C(=O)N[C@@H](CS)C(=O)NCC(N)=O]

To the reactor was placed 0.1 mmol of Fmoc-Rink Amide Resin (0.51 mmol/g, 196 mg) on the Symphony synthesizer, assembled sequences as following procedures sequentially:
“Resin-swelling procedure” was followed;
“Single-coupling procedure” was followed with Fmoc-Gly-OH;
“Single-coupling procedure” was followed with Fmoc-Cys (Trt)-OH;
“Single-coupling procedure” was followed with Fmoc-Leu-OH;
“Single-coupling procedure” was followed with Fmoc-Arg (Pdf)-OH;
“Single-coupling procedure” was followed with Fmoc-Val-OH;
“Single-coupling procedure” was followed with Fmoc-Pro-OH;
“Single-coupling procedure” was followed with Fmoc-Val-OH;
“Single-coupling procedure” was followed with Fmoc-dOrn (Dde)-OH;

To the resulting resin was added a solution of NH₂OH—HCl (1.25 gram, 1.8 mmol) and imidazole (0.918 gram, 1.35 mmol) in NMP (5 mL). The resin was shaken for 90 minutes, washed with DMF (6×5 mL). Repeat this treatment one more time to the resin bound peptide. The resin was washed successively as follows: DMF (3×5.0 mL) and DCM (3×5.0 mL) and then dried.

To the resin bound peptide (0.1 mmol) was added 2 mL of solution of EDC (77 mg, 0.4 mmol) in DMF, followed by DIPEA (0.14 mL, 0.8 mmol) and N-((3,4-difluorophenethyl) carbamothioyl)-2,2,4,6,7-pentamethyl-2,3-dihydrobenzofuran-5-sulfonamide (117 mg, 0.25 mmol, prepared according to procedures from Org. Lett, 2004, 6, 1377-1380). The reaction mixture was stirring overnight. The resin was washed successively as follows: DMF (3×5.0 mL) and DCM (3×5.0 mL) and then dried.

The full linear sequence was then assembled following procedures sequentially:
“Resin-swelling procedure” was followed;
“Single-coupling procedure” was followed with Fmoc-Asp (OtBu)-OH;
“Single-coupling procedure” was followed with Fmoc-Pro-OH;
“Single-coupling procedure” was followed with Fmoc-mGly-OH;
“Single-coupling procedure” was followed with
Fmoc-Bip (4′-2-(3-carbamoyl-1H-indazol-1-yl) acetic acid)-OH;
“Single-coupling procedure” was followed with Fmoc-Bip-OH;
“Single-coupling procedure” was followed with Fmoc-A (Mor)-OH;
“Chloroacetic anhydride coupling” was followed with chloroacetic anhydride;

Finally, the resin bound peptide was cleaved off the resin and deprotected following the Global Deprotection Method procedure to provide linear crude peptide

### Step 2, Macrocyclization:

To the crude linear peptide was added 10% DIEPA in DMF (40 mL). The reaction mixture was shaken overnight. The reaction mixture was filtered through a 0.45-micron filter and concentrated in vacuo. The crude material was purified via preparative LC/MS with the following conditions: Column: Waters XBridge C18, 19×250 mm, 5-μm particles; Mobile Phase A: 5:95 acetonitrile:water with 0.1% TFA; Mobile Phase B: 95:5 acetonitrile: water with 0.1% TFA; Gradient: 17-57% B over 20.5 minutes, 50-100% B over 1 minutes and 5-minute hold at 100% B; Flow: 20 mL/min. Fractions containing the desired product were combined and dried via centrifugal evaporation. It provided 17.6 mg of product, Its estimated purity by LCMS analysis was 97%. Analysis condition 2: Retention time=1.71 min. ESI-MS(+) m/z [M+2H]²⁺: 1091.4.

Examples 365-366, 387-194, 403-409, 424-439, 449, 467-480, 498-501, 504-512, 529, 531-572, 579-585, 598-603, 620-629, and 635-642 were synthesized following similar procedures described for Example 423.

### Preparation of Example 424

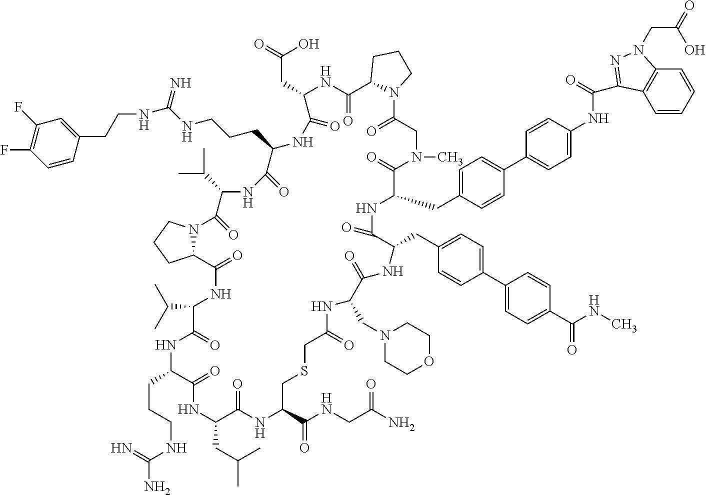 [SMILES: CNC(=O)c1ccc(-c2ccc(C[C@@H]3NC(=O)[C@H](CN4CCOCC4)NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]4CCCN4C(=O)[C@H](C(C)C)NC(=O)[C@@H](CCCNC(=N)NCCc4ccc(F)c(F)c4)NC(=O)[C@H](CC(=O)O)NC(=O)[C@@H]4CCCN4C(=O)CN(C)C(=O)[C@H](Cc4ccc(-c5ccc(NC(=O)c6nn(CC(=O)O)c7ccccc67)cc5)cc4)NC3=O)cc2)cc1]

Example 424 was prepared on a 100 umol scale. It was purified by Preparative LC/MS. The yield of the product was 12.2 mg, and its estimated purity by LCMS analysis was 100%. Analysis condition 1: Retention time=1.5 min. ESI-MS(+) m/z [M+2H] 2+: 1120.

### Preparation of Example 425

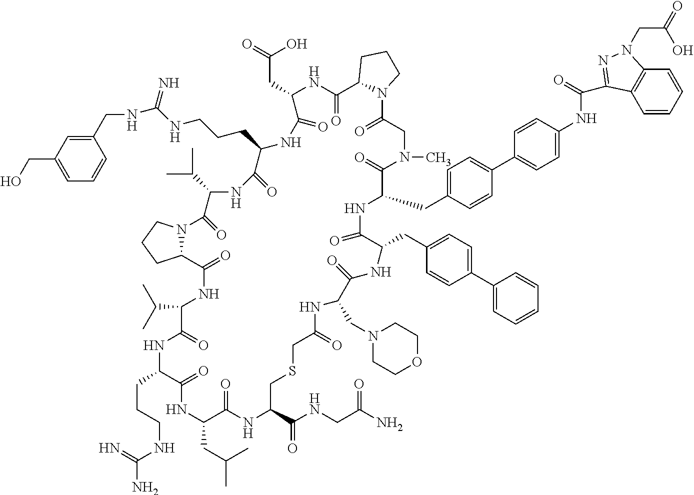 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2cccc(CO)c2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 425 was prepared on a 100 umol scale. It was purified by Preparative LC/MS. The yield of the product was 16.6 mg, and its estimated purity by LCMS analysis was 90.6%. Analysis condition 1: Retention time=1.51 min. ESI-MS(+) m/z [M+2H] 2+: 1081.6.

### Preparation of Example 426

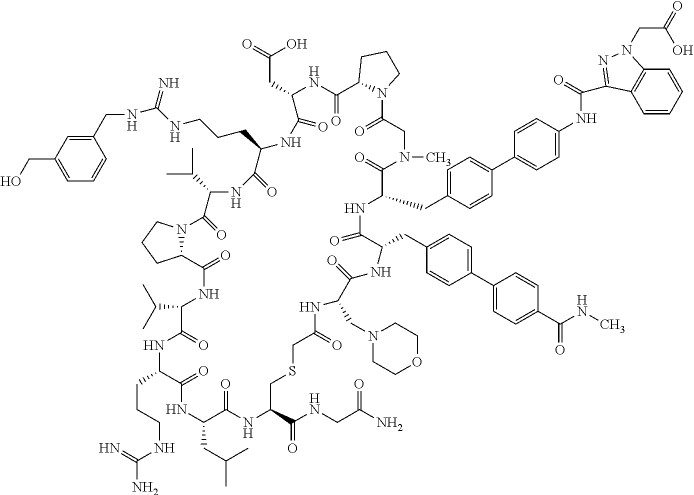 [SMILES: CNC(=O)c1ccc(-c2ccc(C[C@@H]3NC(=O)[C@H](CN4CCOCC4)NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]4CCCN4C(=O)[C@H](C(C)C)NC(=O)[C@@H](CCCNC(=N)NCc4cccc(CO)c4)NC(=O)[C@H](CC(=O)O)NC(=O)[C@@H]4CCCN4C(=O)CN(C)C(=O)[C@H](Cc4ccc(-c5ccc(NC(=O)c6nn(CC(=O)O)c7ccccc67)cc5)cc4)NC3=O)cc2)cc1]

Example 426 was prepared on a 100 umol scale. It was purified by Preparative LC/MS. The yield of the product was 1.5 mg, and its estimated purity by LCMS analysis was 90%. Analysis condition 1: Retention time=1.25 min. ESI-MS(+) m/z [M+2H] 2+: 1110.

### Preparation of Example 427

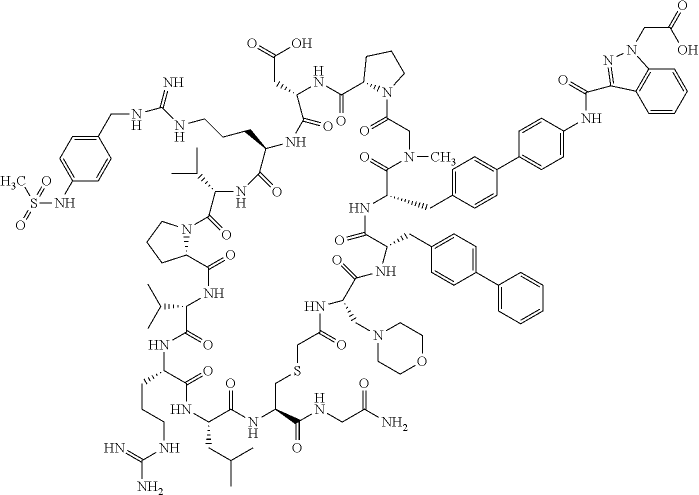 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2ccc(NS(C)(=O)=O)cc2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 427 was prepared on a 100 umol scale. It was purified by Preparative LC/MS. The yield of the product was 8.9 mg, and its estimated purity by LCMS analysis was 94.3%. Analysis condition 1: Retention time=1.39 min. ESI-MS(+) m/z [M+2H] 2+: 1112.9.

### Preparation of Example 428

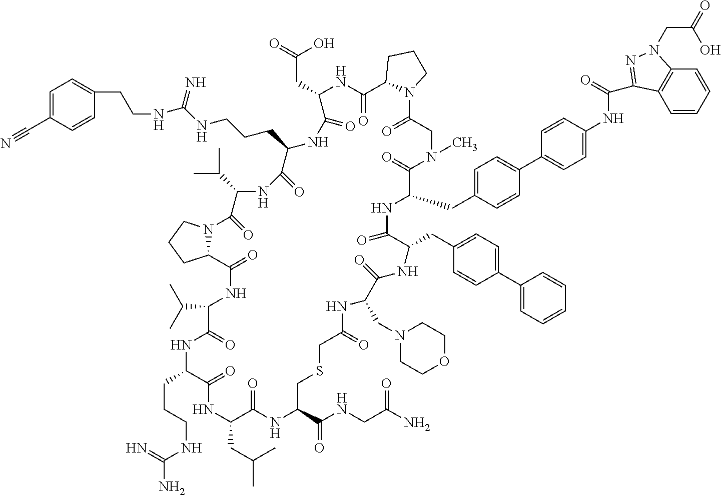 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCc2ccc(C#N)cc2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 428 was prepared on a 50 umol scale. It was purified by Preparative LC/MS. The yield of the product was 17.3 mg, and its estimated purity by LCMS analysis was 91.5%. Analysis condition 1: Retention time=1.46 min. ESI-MS(+) m/z [M+2H] 2+: 1085.8.

### Preparation of Example 429

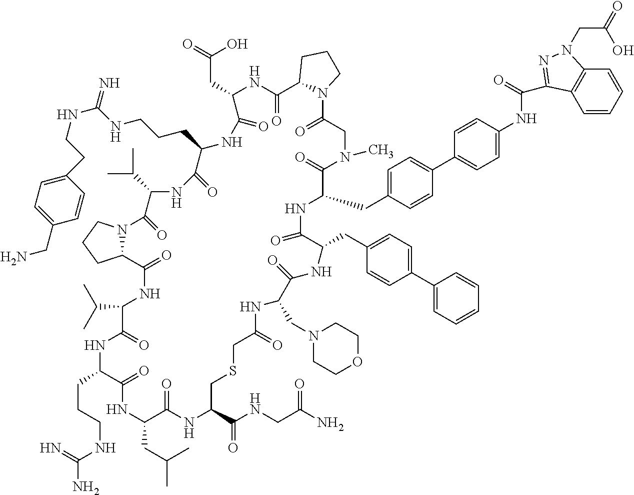 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCc2ccc(CN)cc2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 429 was prepared on a 50 Preparative LC/MS. The yield of the product was 13.5 mg, and its estimated purity by LCMS analysis was 92.2%. Analysis condition 1: Retention time=1.32 min. ESI-MS(+) m/z [M+2H] 2+: 1087.9.

### Preparation of Example 430

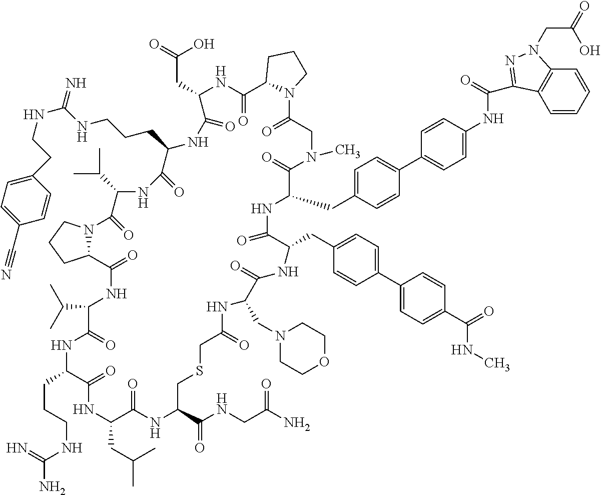 [SMILES: CNC(=O)c1ccc(-c2ccc(C[C@@H]3NC(=O)[C@H](CN4CCOCC4)NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]4CCCN4C(=O)[C@H](C(C)C)NC(=O)[C@@H](CCCNC(=N)NCCc4ccc(C#N)cc4)NC(=O)[C@H](CC(=O)O)NC(=O)[C@@H]4CCCN4C(=O)CN(C)C(=O)[C@H](Cc4ccc(-c5ccc(NC(=O)c6nn(CC(=O)O)c7ccccc67)cc5)cc4)NC3=O)cc2)cc1]

Example 430 was prepared on a 50 umol scale. It was purified by Preparative LC/MS. The yield of the product was 6.5 mg, and its estimated purity by LCMS analysis was 100%. Analysis condition 1: Retention time=1.27 min. ESI-MS(+) m/z [M+2H] 2+: 1114.5.

### Preparation of Example 431

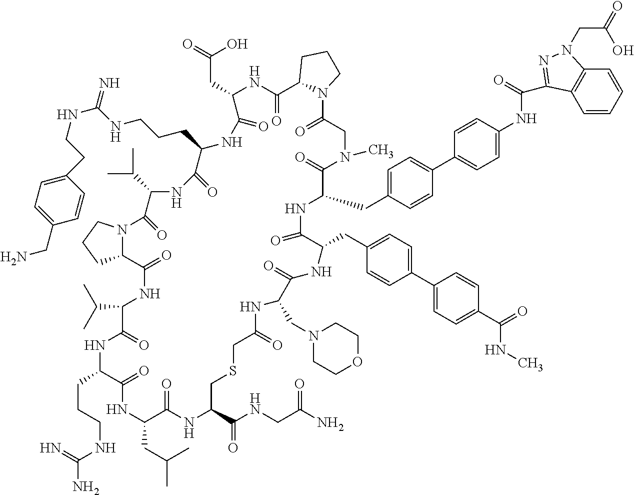 [SMILES: CNC(=O)c1ccc(-c2ccc(C[C@@H]3NC(=O)[C@H](CN4CCOCC4)NC(=O)CSC[C@@H](C(=O)NCC(N)=O)NC(=O)[C@H](CC(C)C)NC(=O)[C@H](CCCNC(=N)N)NC(=O)[C@H](C(C)C)NC(=O)[C@@H]4CCCN4C(=O)[C@H](C(C)C)NC(=O)[C@@H](CCCNC(=N)NCCc4ccc(CN)cc4)NC(=O)[C@H](CC(=O)O)NC(=O)[C@@H]4CCCN4C(=O)CN(C)C(=O)[C@H](Cc4ccc(-c5ccc(NC(=O)c6nn(CC(=O)O)c7ccccc67)cc5)cc4)NC3=O)cc2)cc1]

Example 431 was prepared on a 50 Preparative LC/MS. The yield of the product was 1.7 mg, and its estimated purity by LCMS analysis was 93.9%. Analysis condition 2: Retention time=1.25 min. ESI-MS(+) m/z [M+3H] 3+: 744.8.

### Preparation of Example 432

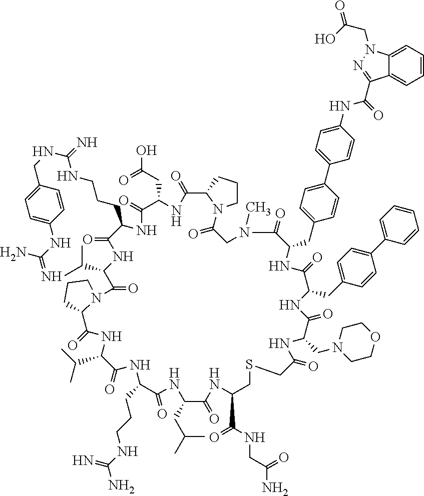 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2ccc(NC(=N)N)cc2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 432 was prepared on a 50 umol scale. It was purified by Preparative LC/MS. The yield of the product was 5.2 mg, and its estimated purity by LCMS analysis was 93.7%. Analysis condition 1: Retention time=1.47 min. ESI-MS(+) m/z [M+2H] 2+: 1094.9.

### Preparation of Example 433

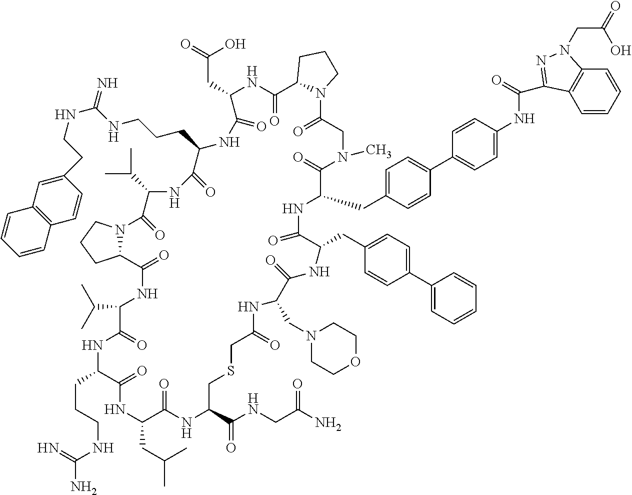 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCc2ccc3ccccc3c2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 433 was prepared on a 50 Preparative LC/MS. The yield of the product was 6.6 mg, and its estimated purity by LCMS analysis was 94.7%. Analysis condition 1: Retention time=1.76 min. ESI-MS(+) m/z [M+2H] 2+: 1098.4.

### Preparation of Example 434

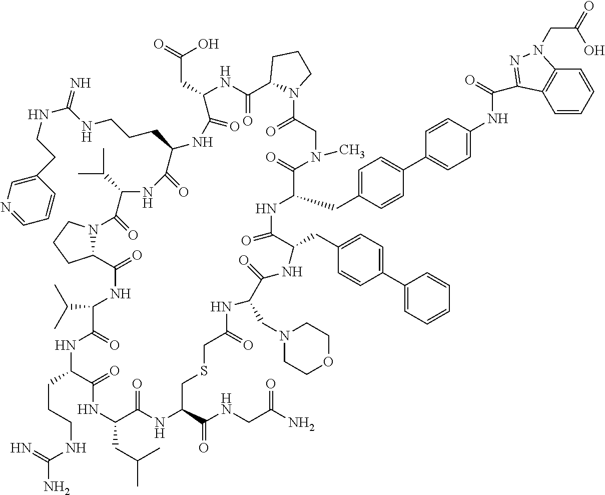 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCc2cccnc2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 434 was prepared on a 50 umol scale. It was purified by Preparative LC/MS. The yield of the product was 1.7 mg, and its estimated purity by LCMS analysis was 100%. Analysis condition 2: Retention time=1.36 min. ESI-MS(+) m/z [M+3H] 3+: 716.4.

### Preparation of Example 435

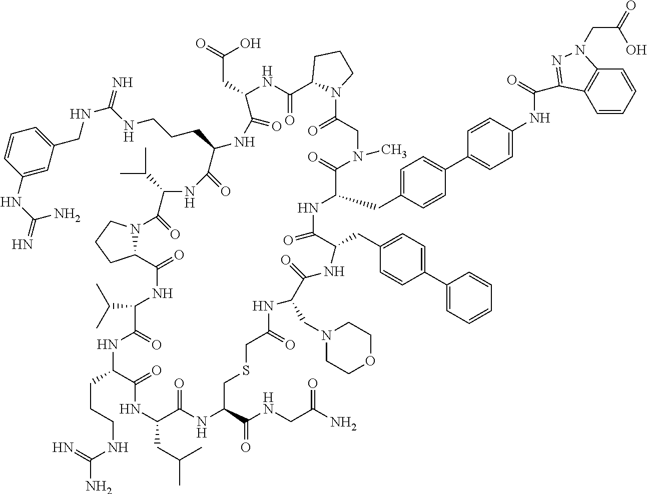 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2cccc(NC(=N)N)c2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 435 was prepared on a 50 Preparative LC/MS. The yield of the product was 5.8 mg, and its estimated purity by LCMS analysis was 90.7%. Analysis condition 1: Retention time=1.37 min. ESI-MS(+) m/z [M+2H] 2+: 1095.1.

### Preparation of Example 436

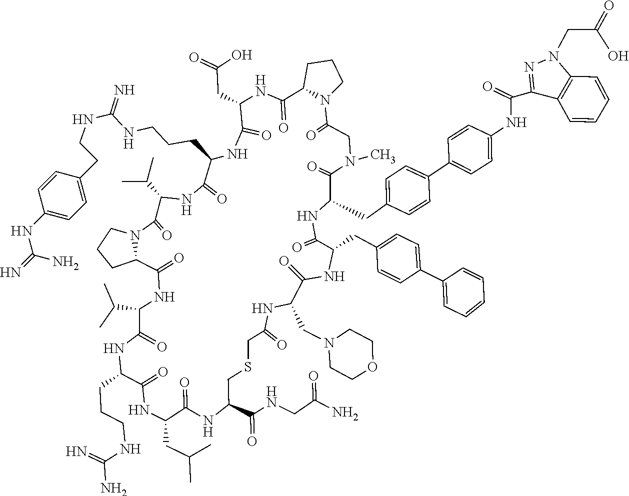 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCc2ccc(NC(=N)N)cc2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 436 was prepared on a 50 umol scale. It was purified by Preparative LC/MS. The yield of the product was 12.5 mg, and its estimated purity by LCMS analysis was 91.5%. Analysis condition 1: Retention time=1.48 min. ESI-MS(+) m/z [M+2H] 2+: 1101.9.

### Preparation of Example 437

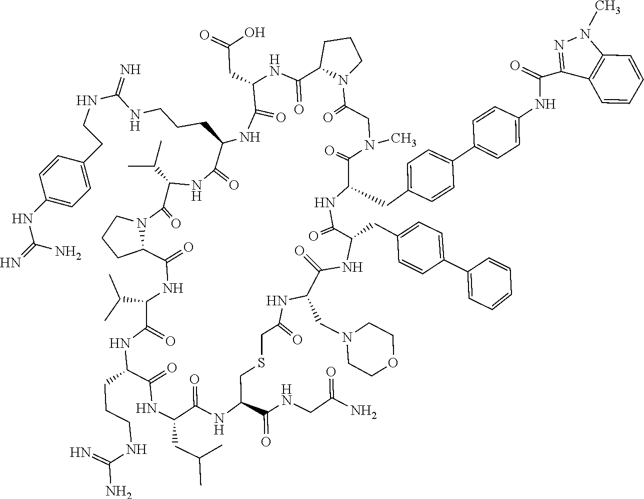 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCc2ccc(NC(=N)N)cc2)NC(=O)[C@H](CC(=O)O)NC(=O)[C@@H]2CCCN2C(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 437 was prepared on a 50 Preparative LC/MS. The yield of the product was 5.5 mg, and its estimated purity by LCMS analysis was 90.3%. Analysis condition 2: Retention time=1.48 min. ESI-MS(+) m/z [M+3H] 3+: 715.8.

### Preparation of Example 438

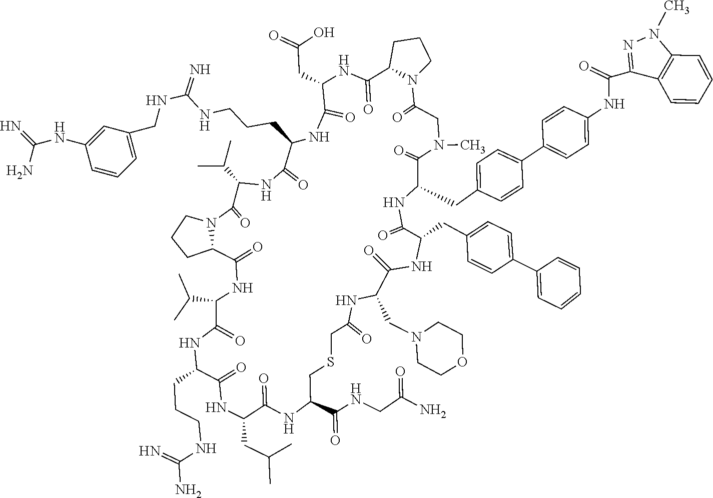 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2cccc(NC(=N)N)c2)NC(=O)[C@H](CC(=O)O)NC(=O)[C@@H]2CCCN2C(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 438 was prepared on a 50 umol scale. It was purified by Preparative LC/MS. The yield of the product was 6.6 mg, and its estimated purity by LCMS analysis was 91.6%. Analysis condition 1: Retention time=1.67 min. ESI-MS(+) m/z [M+2H] 2+: 1072.9.

### Preparation of Example 439

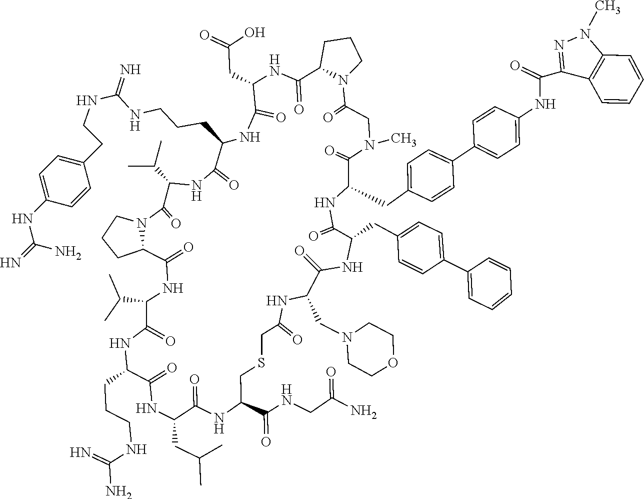 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCCc2ccc(NC(=N)N)cc2)NC(=O)[C@H](CC(=O)O)NC(=O)[C@@H]2CCCN2C(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 439 was prepared on a 50 Preparative LC/MS. The yield of the product was 5.5 mg, and its estimated purity by LCMS analysis was 95.4%. Analysis condition 1: Retention time=1.52 min. ESI-MS(+) m/z [M+3H] 3+: 720.4.

### Preparation of Example 440

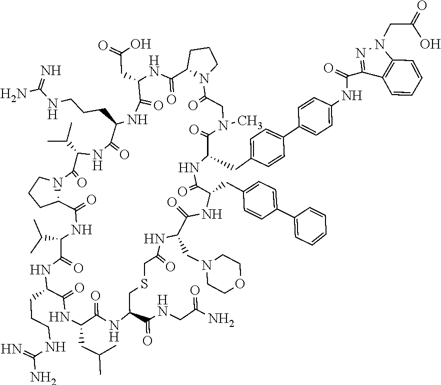 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

### Step 1. Synthesis of the Linear Sequence on Prelude Synthesizer

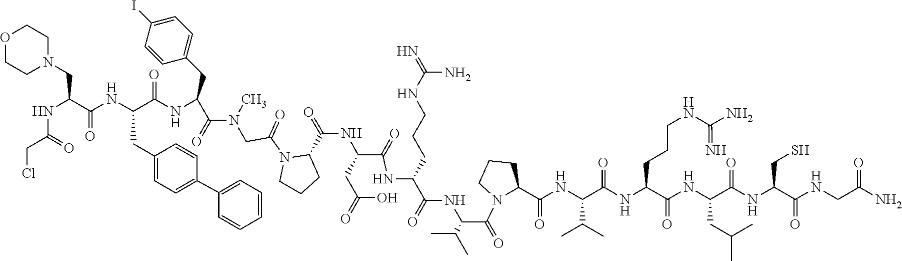 [SMILES: CC(C)C[C@H](NC(=O)[C@H](CCCNC(=N)N)NC(=O)[C@@H](NC(=O)[C@@H]1CCCN1C(=O)[C@@H](NC(=O)[C@@H](CCCNC(=N)N)NC(=O)[C@H](CC(=O)O)NC(=O)[C@@H]1CCCN1C(=O)CN(C)C(=O)[C@H](Cc1ccc(I)cc1)NC(=O)[C@H](Cc1ccc(-c2ccccc2)cc1)NC(=O)[C@H](CN1CCOCC1)NC(=O)CCl)C(C)C)C(C)C)C(=O)N[C@@H](CS)C(=O)NCC(N)=O]

To 45-mL polypropylene solid-phase reaction vessels was added Fmoc-Rink Amide Resin (0.51 mmol/g, 392 mg), and the reaction vessels were placed on the Prelude peptide synthesizer. The following procedures were then performed sequentially: “Resin-swelling procedure” was followed;
“Single-coupling procedure” was followed with Fmoc-Gly-OH;
“Single-coupling procedure” was followed with Fmoc-Cys (Trt)-OH;
“Single-coupling procedure” was followed with Fmoc-Leu-OH;
“Single-coupling procedure” was followed with Fmoc-Arg (Pbf)-OH;
“Single-coupling procedure” was followed with Fmoc-Val-OH;
“Single-coupling procedure” was followed with Fmoc-Pro-OH;
“Single-coupling procedure” was followed with Fmoc-Val-OH;
“Single-coupling procedure” was followed with Fmoc-d-Arg (Pbf)-OH;
“Single-coupling procedure” was followed with Fmoc-Asp (OtBu)-OH;
“Single-coupling procedure” was followed with Fmoc-Pro-OH;
“Single-coupling procedure” was followed with Fmoc-mGly-OH;
“Single-coupling procedure” was followed with Fmoc-Phe (4-I)—OH;
“Single-coupling procedure” was followed with Fmoc-Bip-OH;
“Single-coupling procedure” was followed with Fmoc-A (Mor)-OH;
“Chloroacetic anhydride coupling” was followed with chloroacetic anhydride;

Finally, the resin bound peptide was cleaved off the resin with Global Deprotection Method procedure to provide crude linear peptide

### Step 2. Macrocyclization:

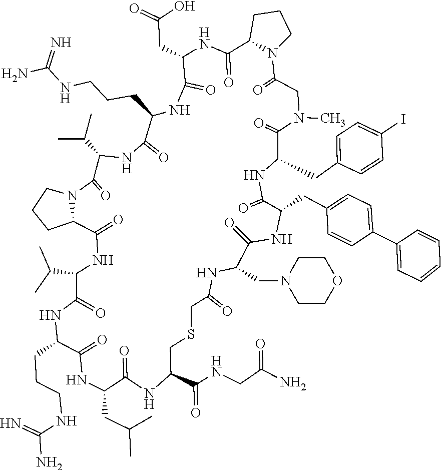 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(I)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

To each crude linear peptide (˜0.05 umol/vessel) from the previous step: was added 40 mL of 10% DIEPA in DMF (40 mL). The reaction mixture was shaken overnight. Each reaction mixture was filtered through a 0.45-micron filter and was concentrated in vacuo.

The crude materials were purified together via a reverse phase ISCO Combi-flash system (300 g ISCO Gold C-18 column) Mobile Phase A: 5:95 acetonitrile:water with 0.1% TFA; Mobile Phase B: 95:5 acetonitrile:water with 0.1% TFA; Gradient: 0-100% B over 35 minutes, then a 5-minute hold at 100% B; Flow: 150 mL/min. Fractions containing the desired product were combined and dried via centrifugal evaporation. The product was then lyophilized overnight (530 mg, 23.4%), and its estimated purity by LCMS analysis was 95%. Analysis condition 4: Retention time=1.36 min; ESI-MS(+) m/z [M+2H]: 937.7.

Step 3. Preparation of example 440: Suzuki-Miyaura Cross-Coupling on Peptide

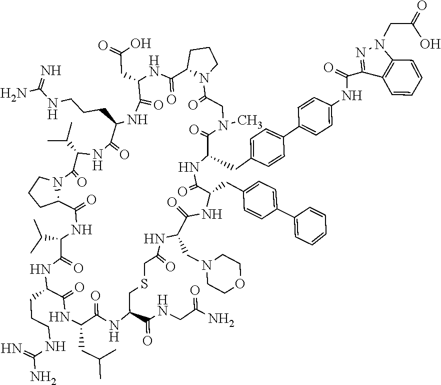 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

A 20 mL glass vial was charged with an aqueous K₂CO₃ solution (7.2 mM, 16.7 mL, 0.12 mmol) and flushed with nitrogen for 30 min. To the solution were added iodide from previous step (0.075 g, 0.040 mmol), 2-(3-((4-boronophenyl) carbamoyl)-1H-indol-1-yl) acetic acid (0.034 g, 0.100 mmol), 1,1′-Bis(Diphenylphosphino) ferrocene-palladium (II) dichloride dichloromethane complex (10 mM in DMF, 0.800 ml, 8.00 umol, 20 mol %) and sodium dodecyl sulfate (0.346 g, 1.201 mmol). The vial was then sealed with a pressure relief cap, and the reaction mixture was stirred under nitrogen at 60° C. in an oil bath for 18 h. The crude reaction was concentrated under reduced pressure. The crude material was purified via preparative LC/MS with the following conditions: Column: Sunfire C18, 250 mm×30 mm, 5-μm particles; Mobile Phase A: 5:95 acetonitrile:water with ammonium acetate; Mobile Phase B: 95:5 acetonitrile:water with ammonium acetate; Gradient: 20-90% B over 25 minutes, then a 0-minute hold at 100% B; Flow Rate: 42 mL/min; Column Temperature: 250° C. Fractions containing the desired product were combined and dried via centrifugal evaporation over 10 hours, 40° C. maximum temperature. The purified, dried product was taken up in 2 ml acetonitrile/water (1:1) and lyophilized. The yield of the product was 7 mg, and its estimated purity by LCMS analysis was 100%. Analysis condition1: Retention time=1.38 min. ESI-MS(+) m/z [M+2H] 2+: 1021.2.

Examples 302-305, 333, 343, 346-348, 382, 398 and 398 were synthesized following similar procedures described for Example 440.

### Preparation of Example 441

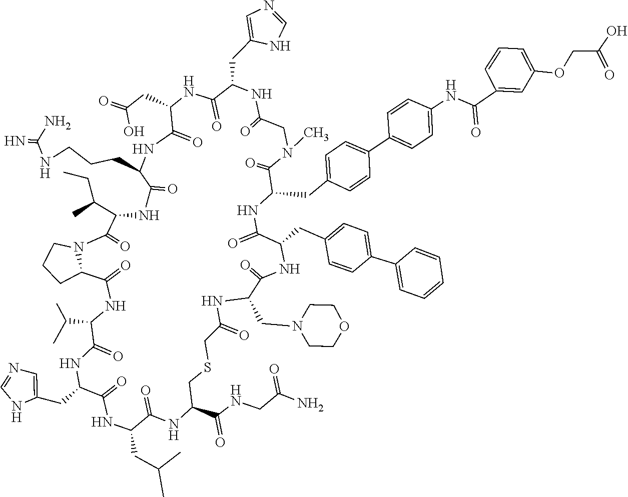 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 441 was prepared on a 50 umol scale. It was purified by Preparative LC/MS. The yield of the product was 23.4 mg, and its estimated purity by LCMS analysis was 95.2%. Analysis condition 2: Retention time=1.22 min. ESI-MS(+) m/z [M+2H] 2+: 1026.8.

### Preparation of Example 442

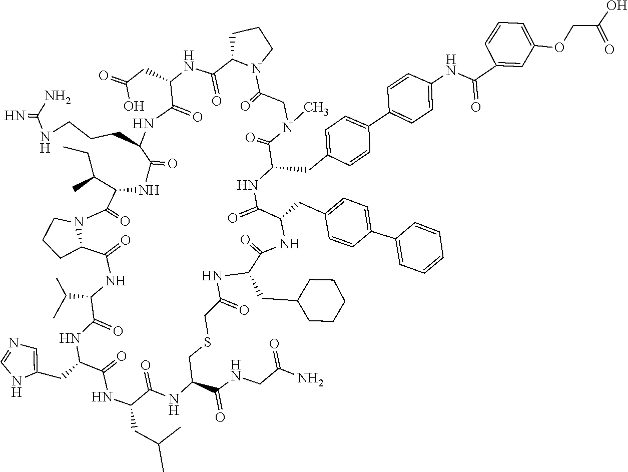 [SMILES: CC[C@H](C)[C@@H]1NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 442 was prepared on a 50 umol scale. It was purified by Preparative LC/MS. The yield of the product was 22 mg, and its estimated purity by LCMS analysis was 96.7%. Analysis condition 2: Retention time=1.47 min. ESI-MS(+) m/z [M+2H] 2+: 1005.4.

### Preparation of Example 443

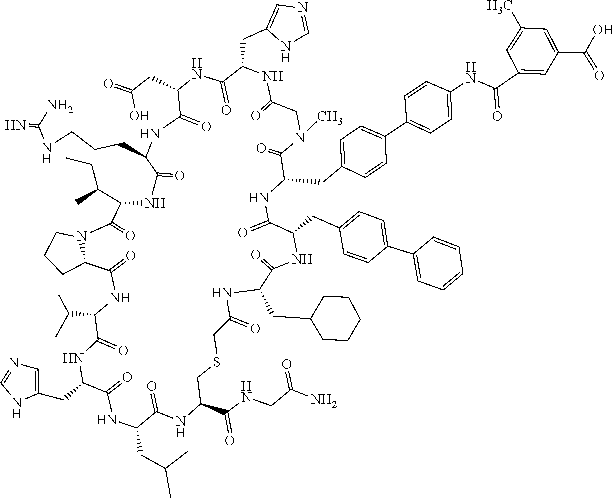 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cc(C)cc(C(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 443 was prepared on a 50 umol scale. It was purified by Preparative LC/MS. The yield of the product was 12.4 mg, and its estimated purity by LCMS analysis was 92.7%. Analysis condition 1: Retention time=1.41 min. ESI-MS(+) m/z [M+2H] 2+: 1017.6.

### Preparation of Example 444

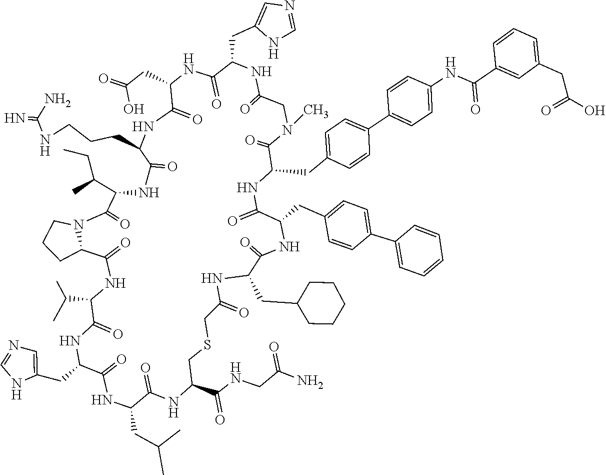 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 444 was prepared on a 50 Preparative LC/MS. The yield of the product was 18.7 mg, and its estimated purity by LCMS analysis was 94.4%. Analysis condition 1: Retention time=1.39 min. ESI-MS(+) m/z [M+2H] 2+: 1017.7.

### Preparation of Example 445

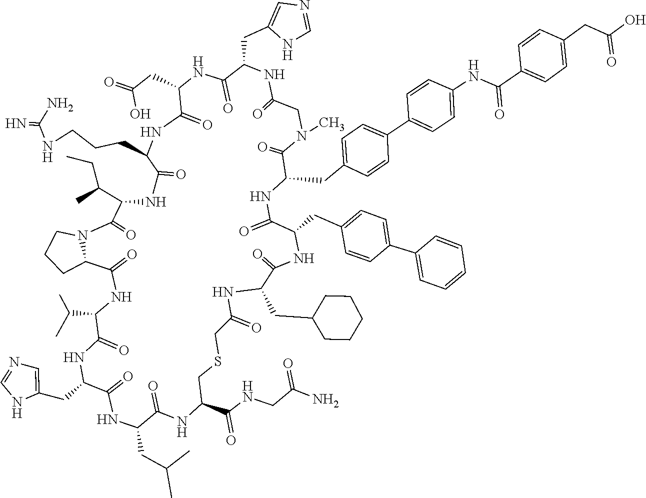 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 445 was prepared on a 50 umol scale. It was purified by Preparative LC/MS. The yield of the product was 21.9 mg, and its estimated purity by LCMS analysis was 95.5%. Analysis condition 1: Retention time=1.37 min. ESI-MS(+) m/z [M+2H] 2+: 1017.5.

### Preparation of Example 446

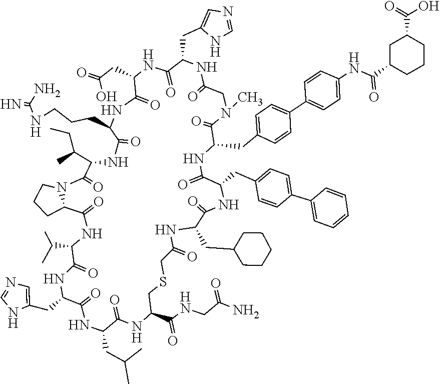 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)[C@H]4CCC[C@@H](C(=O)O)C4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 446 was prepared on a 50 Preparative LC/MS. The yield of the product was 36.9 mg, and its estimated purity by LCMS analysis was 95.6%. Analysis condition 1: Retention time=1.35 min. ESI-MS(+) m/z [M+2H] 2+: 1013.4.

### Preparation of Example 447

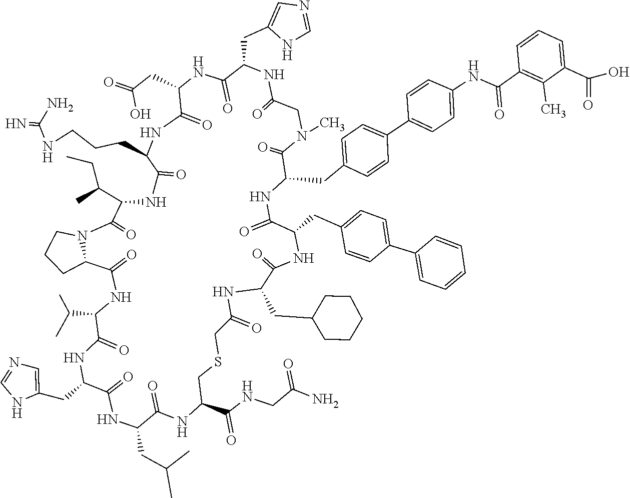 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(C(=O)O)c4C)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 447 was prepared on a 50 umol scale. It was purified by Preparative LC/MS. The yield of the product was 9.8 mg, and its estimated purity by LCMS analysis was 93.6%. Analysis condition 2: Retention time=1.42 min. ESI-MS(+) m/z [M+2H] 2+: 1017.4.

### Preparation of Example 448

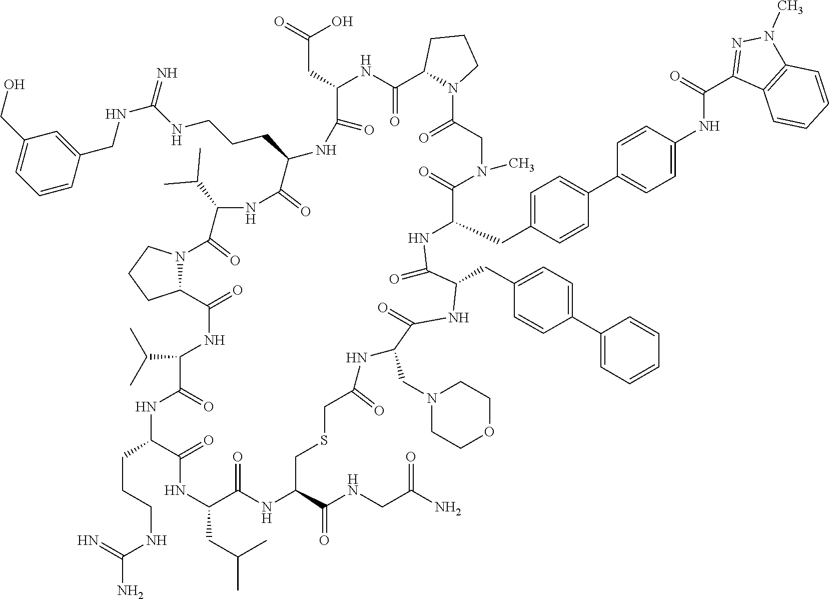 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2cccc(CO)c2)NC(=O)[C@H](CC(=O)O)NC(=O)[C@@H]2CCCN2C(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 448 was prepared on a 50 Preparative LC/MS. The yield of the product was 8.6 mg, and its estimated purity by LCMS analysis was 99.1%. Analysis condition 2: Retention time=1.51 min. ESI-MS(+) m/z [M+2H] 2+: 1059.6.

### Preparation of Example 449

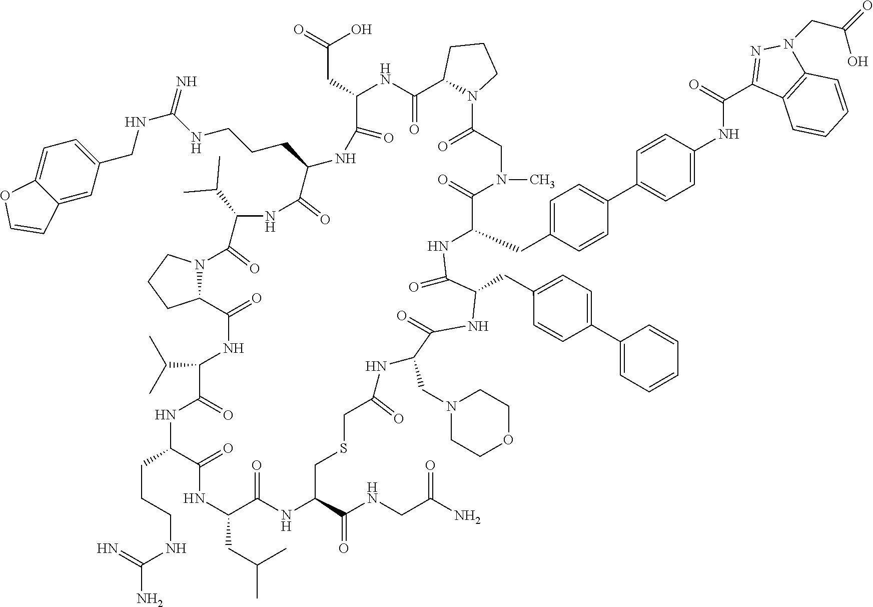 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2ccc3occc3c2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 449 was prepared on a 50 umol scale. It was purified by Preparative LC/MS. The yield of the product was 6.3 mg, and its estimated purity by LCMS analysis was 93.8%. Analysis condition 2: Retention time=1.52 min. ESI-MS(+) m/z [M+2H] 2+: 1086.5.

### Preparation of Example 450

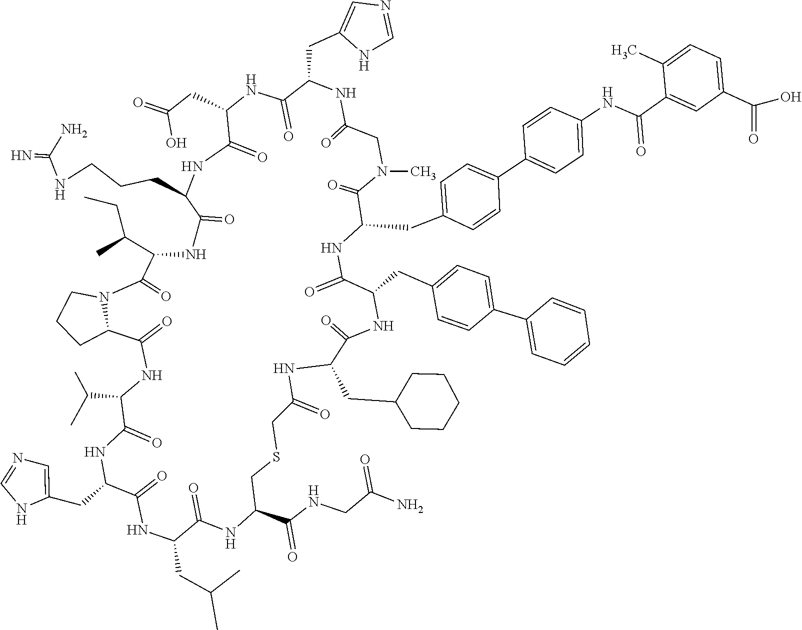 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cc(C(=O)O)ccc4C)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 450 was prepared on a 50 Preparative LC/MS. The yield of the product was 10.4 mg, and its estimated purity by LCMS analysis was 93.3%. Analysis condition 2: Retention time=1.44 min. ESI-MS(+) m/z [M+2H] 2+: 1017.4.

### Preparation of Example 451

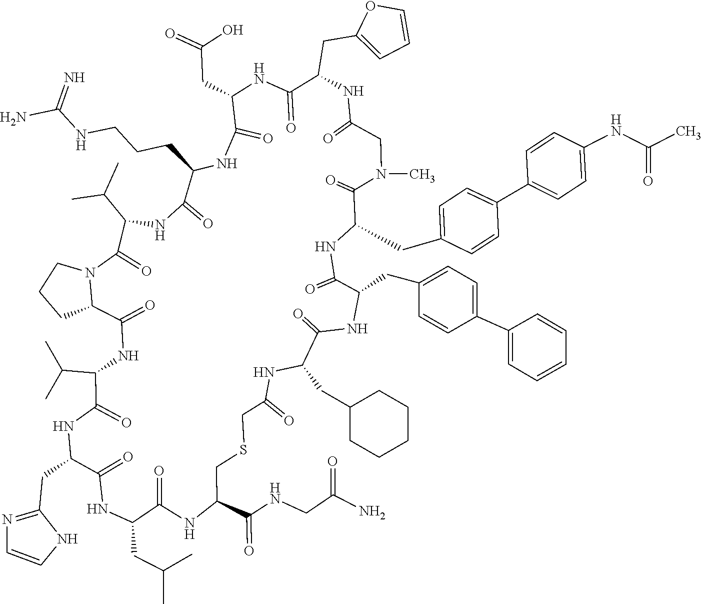 [SMILES: CC(=O)Nc1ccc(-c2ccc(C[C@@H]3NC(=O)[C@H](Cc4ccc(-c5ccccc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4ncc[nH]4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccco4)NC(=O)CN(C)C3=O)cc2)cc1]

Example 451 was prepared on a 50 umol scale. It was purified by Preparative LC/MS. The yield of the product was 37.7 mg, and its estimated purity by LCMS analysis was 95.3%. Analysis condition 1: Retention time=1.58 min. ESI-MS(+) m/z [M+H]⁺: 1899.8.

### Preparation of Example 452

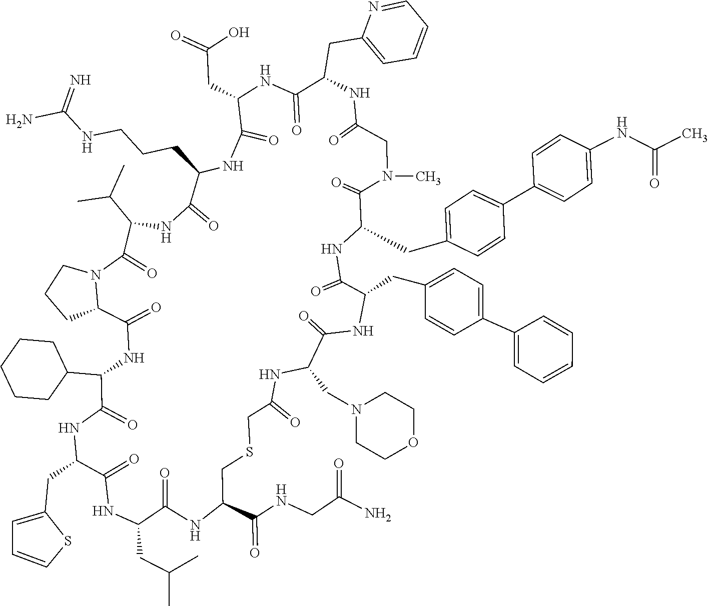 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cccs4)NC(=O)[C@H](C4CCCCC4)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 452 was prepared on a 50 umol scale. It was purified by Preparative LC/MS. The yield of the product was 37.7 mg, and its estimated purity by LCMS analysis was 98.7%. Analysis condition 1: Retention time=2.2 min. ESI-MS(+) m/z [M+H]⁺: 1970.4.

### Preparation of Example 453

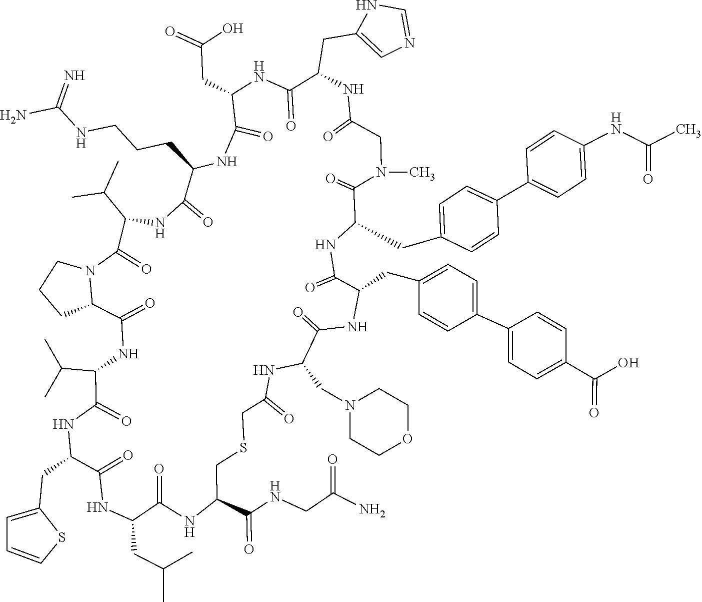 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cccs4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4cnc[nH]4)NC(=O)CN(C)C3=O)cc2)cc1]

Example 453 was prepared on a 50 umol scale. It was purified by Preparative LC/MS. The yield of the product was 30.4 mg, and its estimated purity by LCMS analysis was 95.4%. Analysis condition 1: Retention time=1.19 min. ESI-MS(+) m/z [M+H]⁺: 1961.7.

### Preparation of Example 454

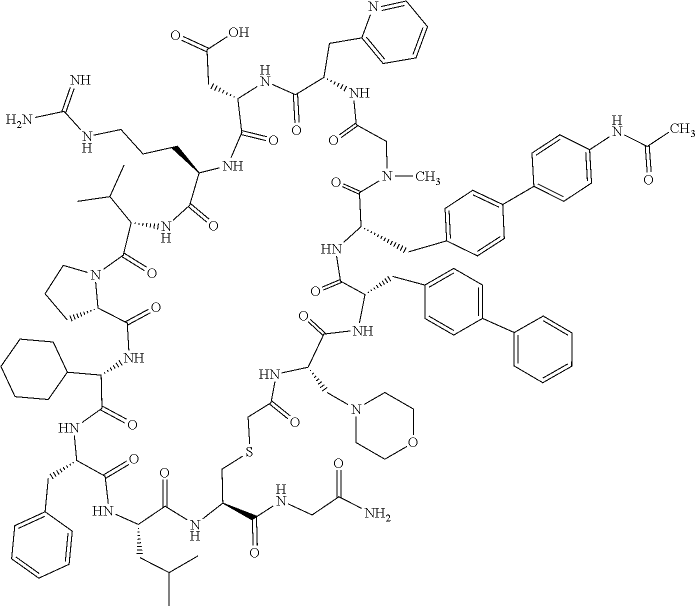 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ccccc4)NC(=O)[C@H](C4CCCCC4)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 454 was prepared on a 50 umol scale. It was purified by Preparative LC/MS. The yield of the product was 48.5 mg, and its estimated purity by LCMS analysis was 96.8%. Analysis condition 1: Retention time=2.24 min. ESI-MS(+) m/z [M+H]⁺: 1963.8.

### Preparation of Example 455

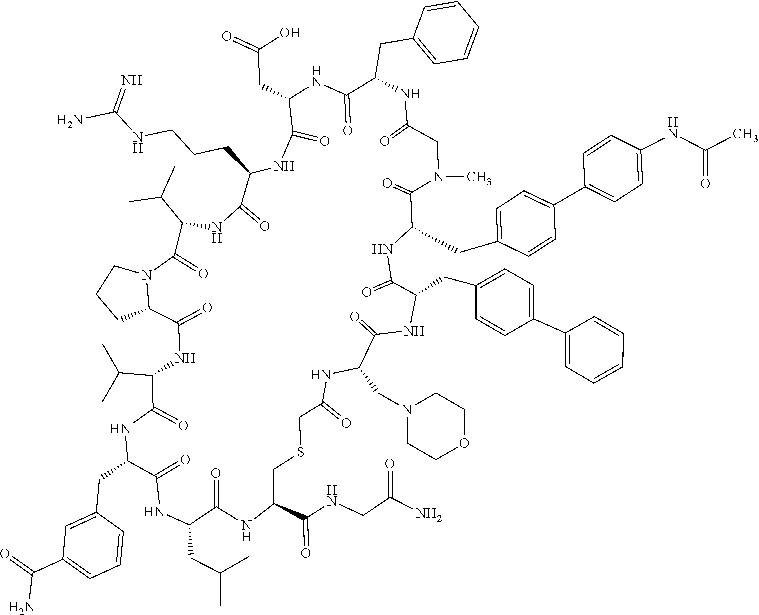 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cccc(C(N)=O)c4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccc4)NC(=O)CN(C)C3=O)cc2)cc1]

Example 455 was prepared on a 50 umol scale. It was purified by Preparative LC/MS. The yield of the product was 48.2 mg, and its estimated purity by LCMS analysis was 95.1%. Analysis condition 1: Retention time=1.47 min. ESI-MS(+) m/z [M+H]⁺: 1965.4.

### Preparation of Example 456

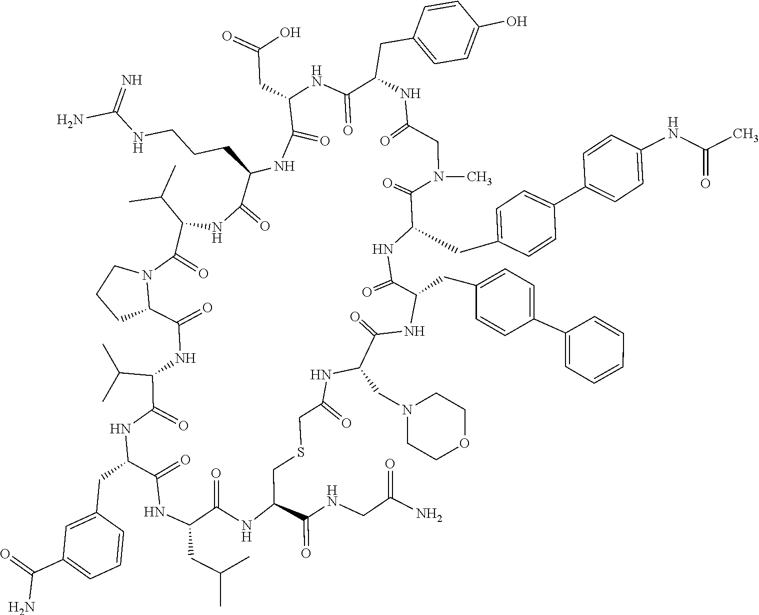 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cccc(C(N)=O)c4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccc(O)cc4)NC(=O)CN(C)C3=O)cc2)cc1]

Example 456 was prepared on a 50 umol scale. It was purified by Preparative LC/MS. The yield of the product was 41.1 mg, and its estimated purity by LCMS analysis was 95.9%. Analysis condition 2: Retention time=1.37 min. ESI-MS(+) m/z [M+2H] 2+: 991.4.

### Preparation of Example 457

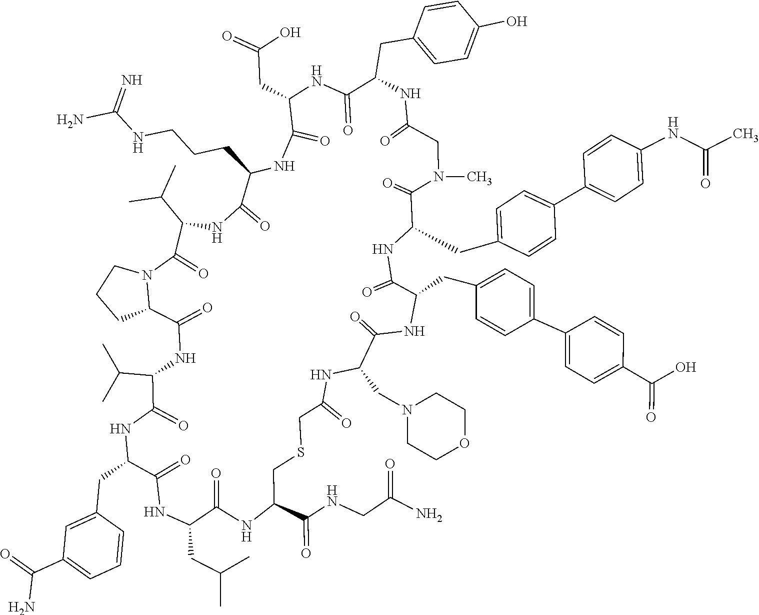 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cccc(C(N)=O)c4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccc(O)cc4)NC(=O)CN(C)C3=O)cc2)cc1]

Example 457 was prepared on a 50 umol scale. It was purified by Preparative LC/MS. The yield of the product was 32.5 mg, and its estimated purity by LCMS analysis was 95.3%. Analysis condition 1: Retention time=1.63 min. ESI-MS(+) m/z [M+2H] 2+: 1013.2.

### Preparation of Example 458

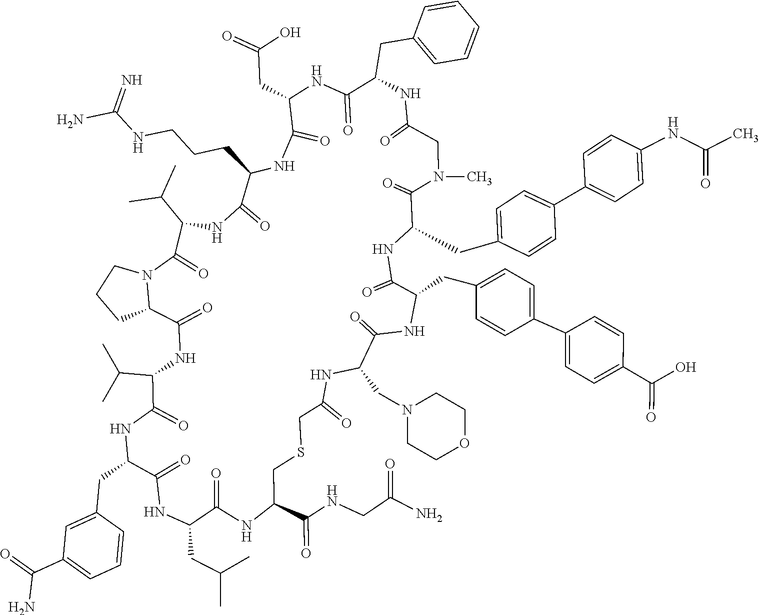 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cccc(C(N)=O)c4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccc4)NC(=O)CN(C)C3=O)cc2)cc1]

Example 458 was prepared on a 50 umol scale. It was purified by Preparative LC/MS. The yield of the product was 30.4 mg, and its estimated purity by LCMS analysis was 91.2%. Analysis condition 1: Retention time=1.34 min. ESI-MS(+) m/z [M+2H] 2+: 1005.4.

### Preparation of Example 459

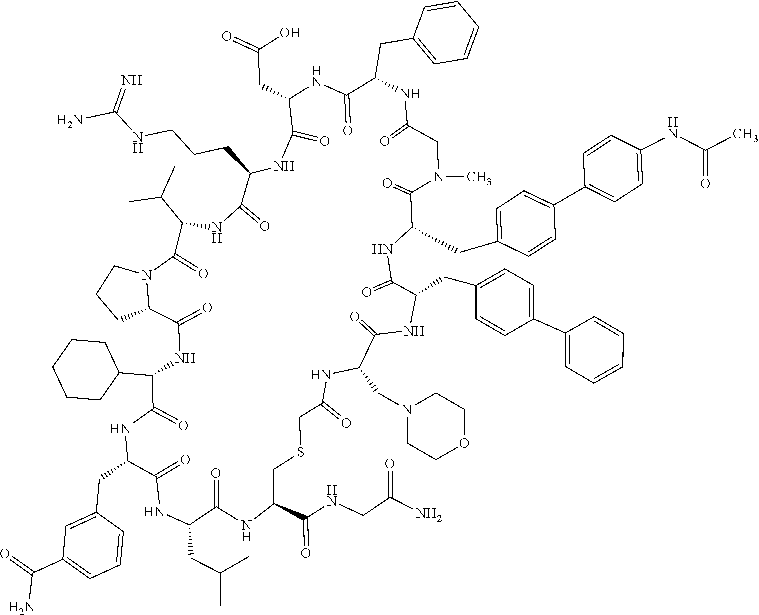 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cccc(C(N)=O)c4)NC(=O)[C@H](C4CCCCC4)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccc4)NC(=O)CN(C)C3=O)cc2)cc1]

Example 459 was prepared on a 50 umol scale. It was purified by Preparative LC/MS. The yield of the product was 38.3 mg, and its estimated purity by LCMS analysis was 95.5%. Analysis condition 1: Retention time=1.58 min. ESI-MS(+) m/z [M+2H] 2+: 1003.3.

### Preparation of Example 460

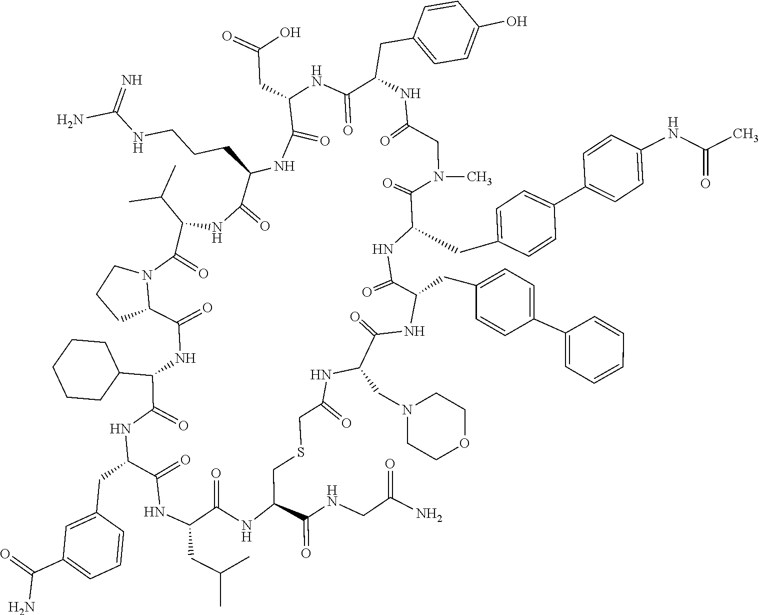 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cccc(C(N)=O)c4)NC(=O)[C@H](C4CCCCC4)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccc(O)cc4)NC(=O)CN(C)C3=O)cc2)cc1]

Example 460 was prepared on a 50 umol scale. It was purified by Preparative LC/MS. The yield of the product was 42.6 mg, and its estimated purity by LCMS analysis was 95.1%. Analysis condition 1: Retention time=1.54 min. ESI-MS(+) m/z [M+2H] 2+: 1011.4.

### Preparation of Example 461

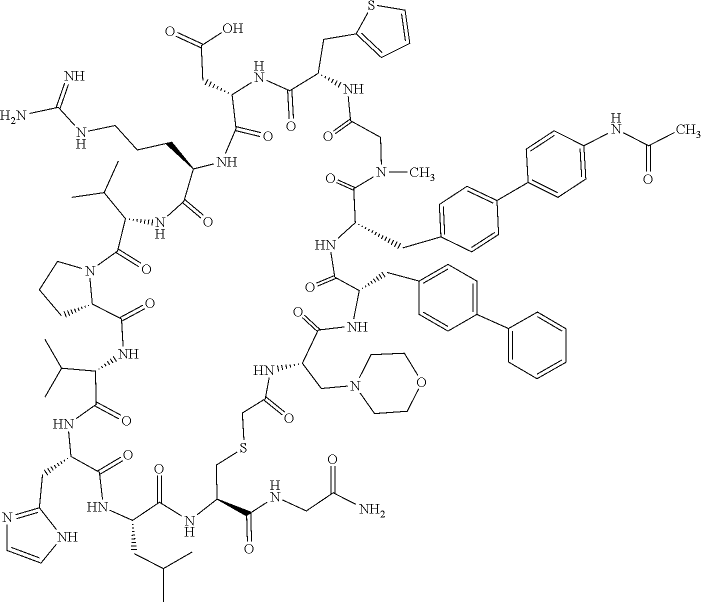 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ncc[nH]4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4cccs4)NC(=O)CN(C)C3=O)cc2)cc1]

Example 461 was prepared on a 50 umol scale. It was purified by Preparative LC/MS. The yield of the product was 49.3 mg, and its estimated purity by LCMS analysis was 97.2%. Analysis condition 1: Retention time=1.47 min. ESI-MS(+) m/z [M+2H] 2+: 960.2.

### Preparation of Example 462

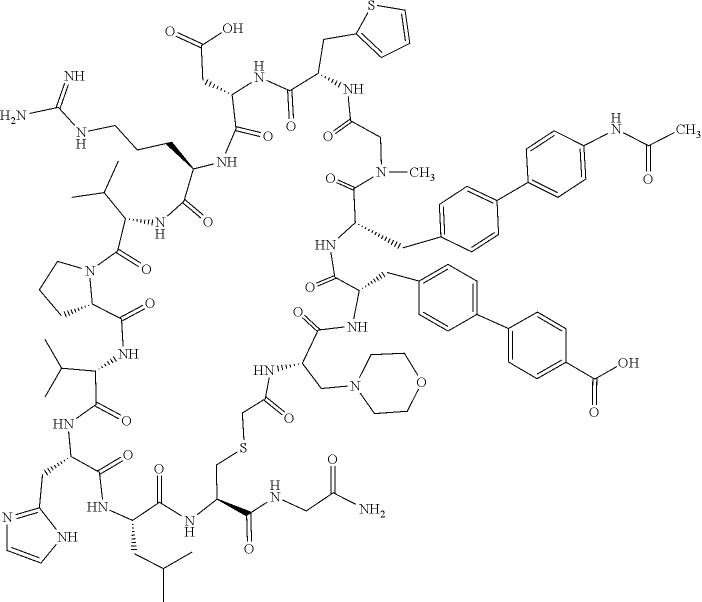 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ncc[nH]4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4cccs4)NC(=O)CN(C)C3=O)cc2)cc1]

Example 462 was prepared on a 50 umol scale. It was purified by Preparative LC/MS. The yield of the product was 28.7 mg, and its estimated purity by LCMS analysis was 97.3%. Analysis condition 1: Retention time=1.31 min. ESI-MS(+) m/z [M+H]⁺: 1961.7.

### Preparation of Example 463

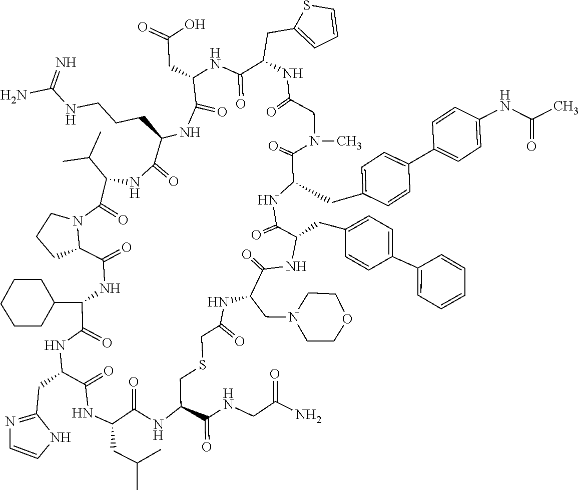 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ncc[nH]4)NC(=O)[C@H](C4CCCCC4)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4cccs4)NC(=O)CN(C)C3=O)cc2)cc1]

Example 463 was prepared on a 50 umol scale. It was purified by Preparative LC/MS. The yield of the product was 35.9 mg, and its estimated purity by LCMS analysis was 93.9%. Analysis condition 1: Retention time=2.19 min. ESI-MS(+) m/z [M+H]⁺: 1959.3.

### Preparation of Example 464

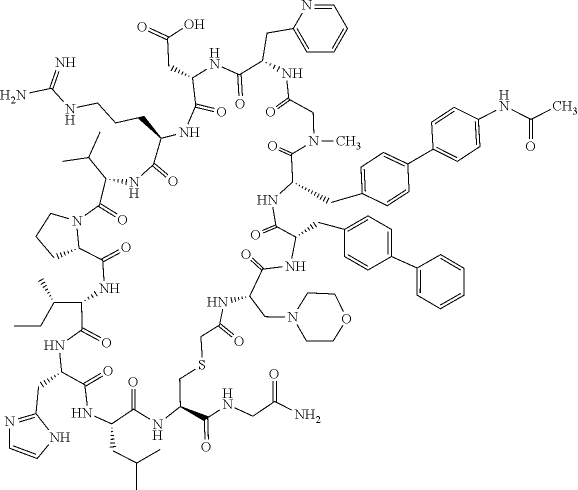 [SMILES: CC[C@H](C)[C@@H]1NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(C)=O)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2ncc[nH]2)NC1=O]

Example 464 was prepared on a 50 umol scale. It was purified by Preparative LC/MS. The yield of the product was 41 mg, and its estimated purity by LCMS analysis was 95.8%. Analysis condition 1: Retention time=1.54 min. ESI-MS(+) m/z [M+H]⁺: 1926.7.

### Preparation of Example 465

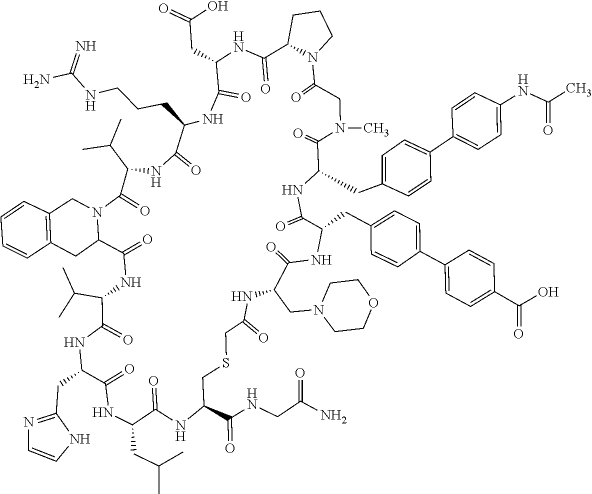 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4ncc[nH]4)NC(=O)[C@H](C(C)C)NC(=O)C4Cc5ccccc5CN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 465 was prepared on a 50 umol scale. It was purified by Preparative LC/MS. The yield of the product was 24.5 mg, and its estimated purity by LCMS analysis was 96.1%. Analysis condition 1: Retention time=1.2 min. ESI-MS(+) m/z [M+H]⁺: 1965.8.

### Preparation of Example 466

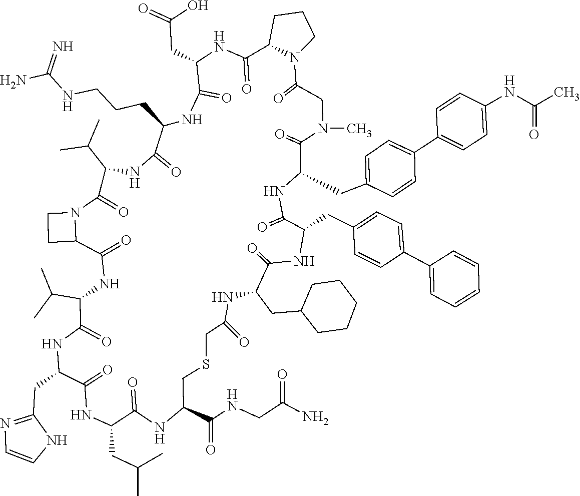 [SMILES: CC(=O)Nc1ccc(-c2ccc(C[C@@H]3NC(=O)[C@H](Cc4ccc(-c5ccccc5)cc4)NC(=O)[C@H](CC4CCCCC4)NC(=O)CSC[C@@H](C(=O)NCC(N)=O)NC(=O)[C@H](CC(C)C)NC(=O)[C@H](Cc4ncc[nH]4)NC(=O)[C@H](C(C)C)NC(=O)C4CCN4C(=O)[C@H](C(C)C)NC(=O)[C@@H](CCCNC(=N)N)NC(=O)[C@H](CC(=O)O)NC(=O)[C@@H]4CCCN4C(=O)CN(C)C3=O)cc2)cc1]

Example 466 was prepared on a 50 umol scale. It was purified by Preparative LC/MS. The yield of the product was 51.4 mg, and its estimated purity by LCMS analysis was 96.6%. Analysis condition 2: Retention time=1.39 min. ESI-MS(+) m/z [M+H]⁺: 1845.

### Preparation of Example 467

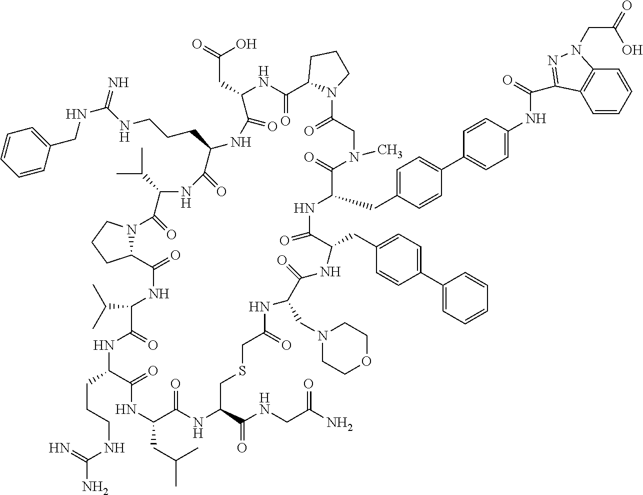 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2ccccc2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 467 was prepared on a 50 umol scale. It was purified by Preparative LC/MS. The yield of the product was 3 mg, and its estimated purity by LCMS analysis was 98.2%. Analysis condition 1: Retention time=1.42 min. ESI-MS(+) m/z [M+2H] 2+: 1066.9.

### Preparation of Example 468

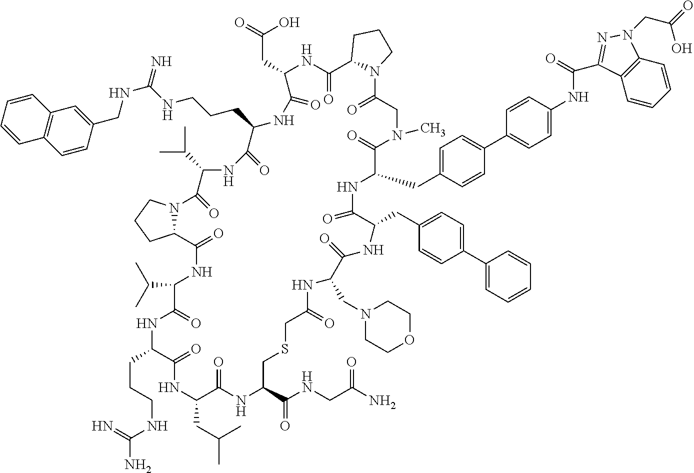 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2ccc3ccccc3c2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 468 was prepared on a 50 Preparative LC/MS. The yield of the product was 3.6 mg, and its estimated purity by LCMS analysis was 97.9%. Analysis condition 2: Retention time=1.6 min. ESI-MS(+) m/z [M+2H] 2+: 1091.7.

### Preparation of Example 469

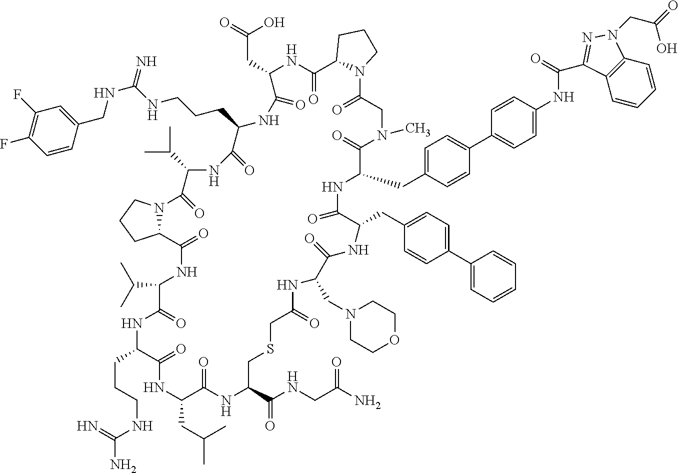 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2ccc(F)c(F)c2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 469 was prepared on a 50 umol scale. It was purified by Preparative LC/MS. The yield of the product was 3.3 mg, and its estimated purity by LCMS analysis was 100%. Analysis condition 1: Retention time=1.46 min. ESI-MS(+) m/z [M+2H] 2+: 1084.9.

### Preparation of Example 470

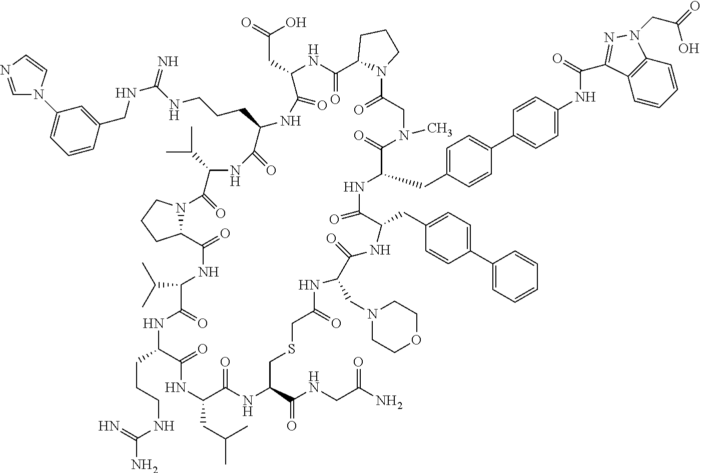 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2cccc(-n3ccnc3)c2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 470 was prepared on a 50 Preparative LC/MS. The yield of the product was 5.8 mg, and its estimated purity by LCMS analysis was 92.5%. Analysis condition 2: Retention time=1.4 min. ESI-MS(+) m/z [M+3H] 3+: 733.2.

### Preparation of Example 471

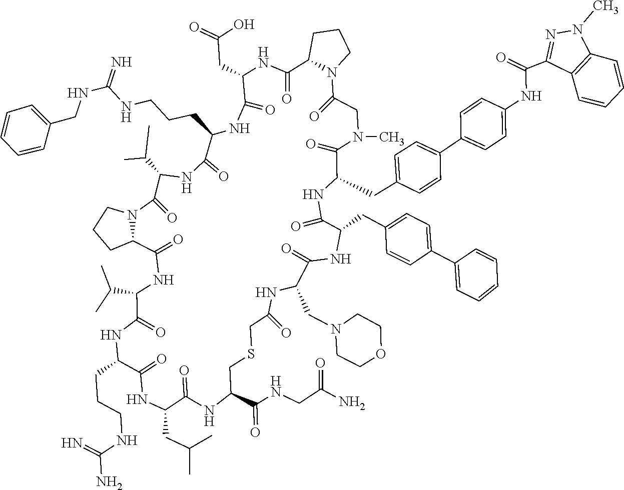 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2ccccc2)NC(=O)[C@H](CC(=O)O)NC(=O)[C@@H]2CCCN2C(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 471 was prepared on a 50 umol scale. It was purified by Preparative LC/MS. The yield of the product was 11 mg, and its estimated purity by LCMS analysis was 93.5%. Analysis condition 1: Retention time=1.69 min. ESI-MS(+) m/z [M+2H] 2+: 1044.3.

### Preparation of Example 472

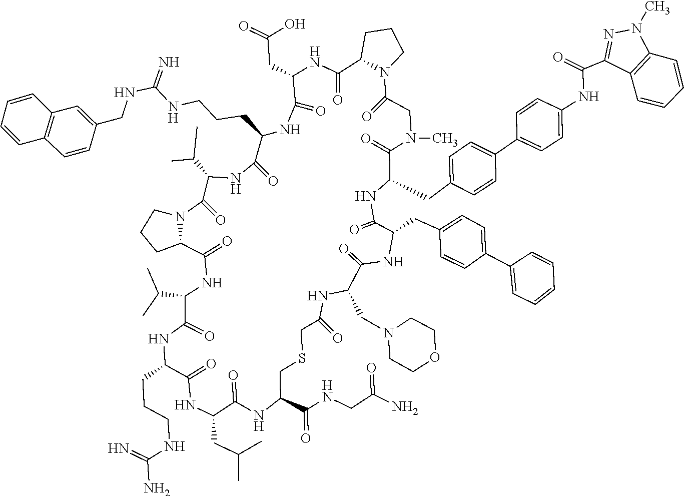 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2ccc3ccccc3c2)NC(=O)[C@H](CC(=O)O)NC(=O)[C@@H]2CCCN2C(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 472 was prepared on a 50 Preparative LC/MS. The yield of the product was 16.6 mg, and its estimated purity by LCMS analysis was 91.6%. Analysis condition 2: Retention time=1.73 min. ESI-MS(+) m/z [M+2H] 2+: 1069.4.

### Preparation of Example 473

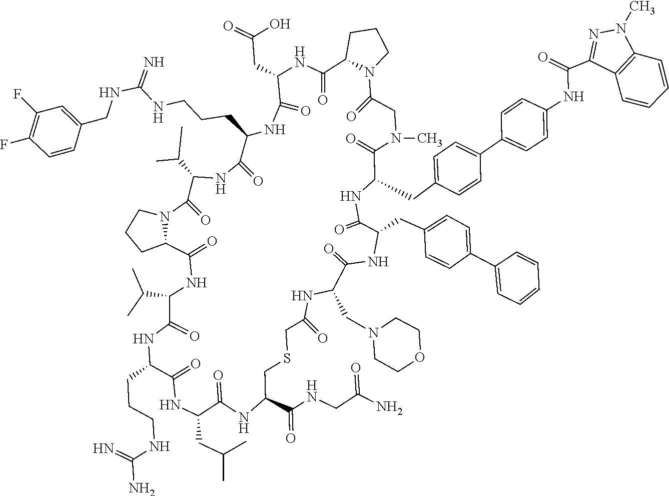 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2ccc(F)c(F)c2)NC(=O)[C@H](CC(=O)O)NC(=O)[C@@H]2CCCN2C(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 473 was prepared on a 50 umol scale. It was purified by Preparative LC/MS. The yield of the product was 19 mg, and its estimated purity by LCMS analysis was 93.1%. Analysis condition 1: Retention time=1.72 min. ESI-MS(+) m/z [M+2H] 2+: 1062.3.

### Preparation of Example 474

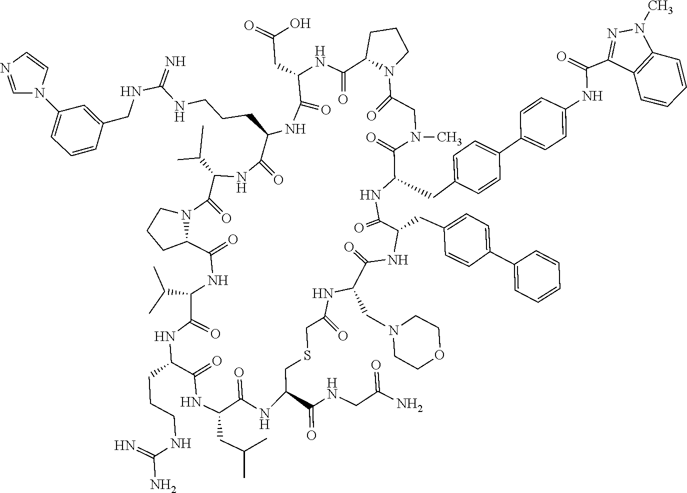 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2cccc(-n3ccnc3)c2)NC(=O)[C@H](CC(=O)O)NC(=O)[C@@H]2CCCN2C(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 474 was prepared on a 50 Preparative LC/MS. The yield of the product was 15.1 mg, and its estimated purity by LCMS analysis was 95%. Analysis condition 1: Retention time=1.59 min. ESI-MS(+) m/z [M+3H] [M+3H] 3+: 718.6.

### Preparation of Example 475

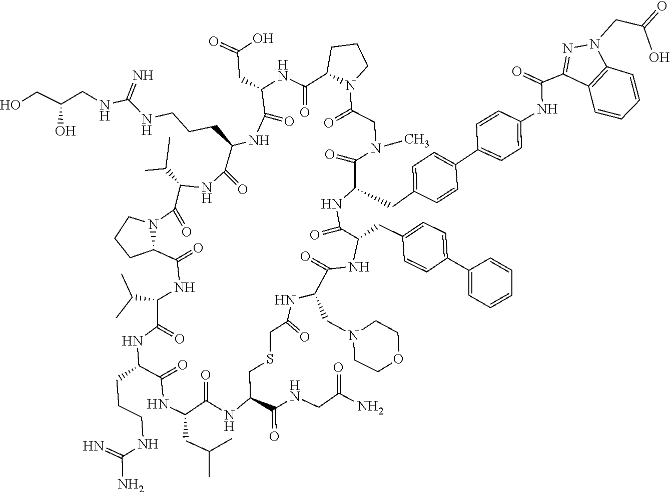 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NC[C@H](O)CO)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 475 was prepared on a 50 umol scale. It was purified by Preparative LC/MS. The yield of the product was 2.1 mg, and its estimated purity by LCMS analysis was 100%. Analysis condition 1: Retention time=1.31 min. ESI-MS(+) m/z [M+2H] [M+2H] 2+: 1058.4.

### Preparation of Example 476

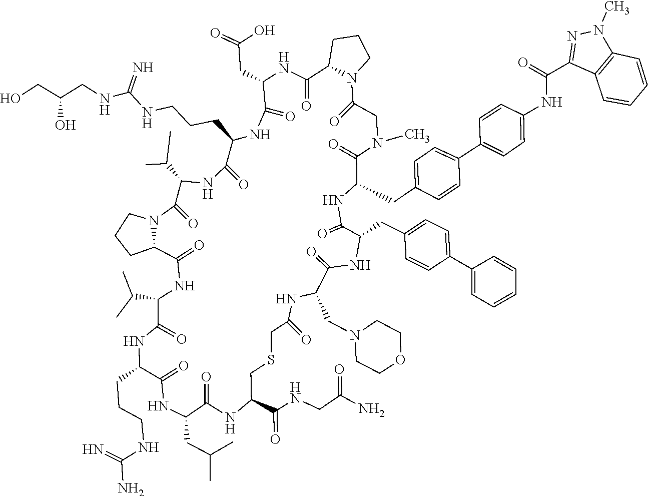 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NC[C@H](O)CO)NC(=O)[C@H](CC(=O)O)NC(=O)[C@@H]2CCCN2C(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 476 was prepared on a 50 Preparative LC/MS. The yield of the product was 5.8 mg, and its estimated purity by LCMS analysis was 100%. Analysis condition 2: Retention time=1.52 min. ESI-MS(+) m/z [M+2H] [M+2H] 2+: 1036.7.

### Preparation of Example 477

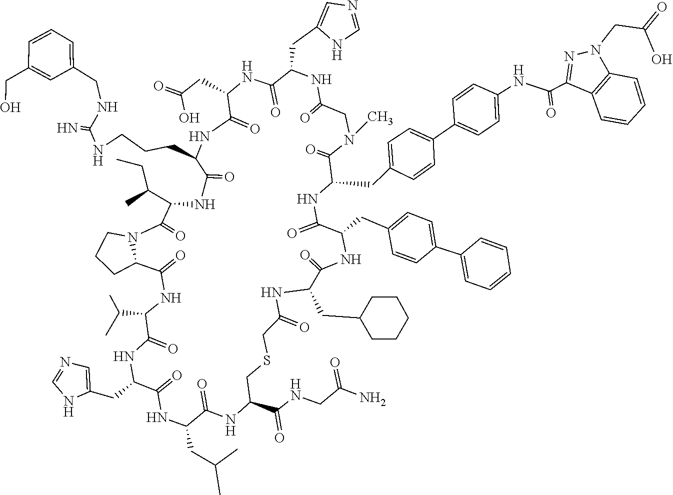 [SMILES: CC[C@H](C)[C@@H]1NC(=O)[C@@H](CCCNC(=N)NCc2cccc(CO)c2)NC(=O)[C@H](CC(=O)O)NC(=O)[C@H](Cc2cnc[nH]2)NC(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 477 was prepared on a 50 umol scale. It was purified by Preparative LC/MS. The yield of the product was 1.4 mg, and its estimated purity by LCMS analysis was 92.1%. Analysis condition 1: Retention time=1.49 min. ESI-MS(+) m/z [M+2H] 2+: 1097.8.

### Preparation of Example 478

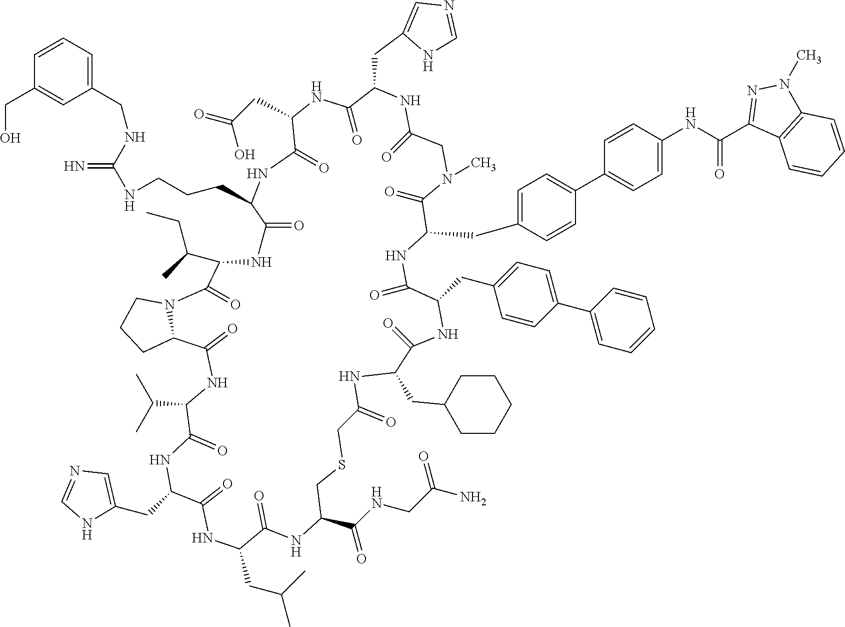 [SMILES: CC[C@H](C)[C@@H]1NC(=O)[C@@H](CCCNC(=N)NCc2cccc(CO)c2)NC(=O)[C@H](CC(=O)O)NC(=O)[C@H](Cc2cnc[nH]2)NC(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 478 was prepared on a 50 Preparative LC/MS. The yield of the product was 15.6 mg, and its estimated purity by LCMS analysis was 100%. Analysis condition 2: Retention time=1.61 min. ESI-MS(+) m/z [M+3H] 3+: 717.3.

### Preparation of Example 479

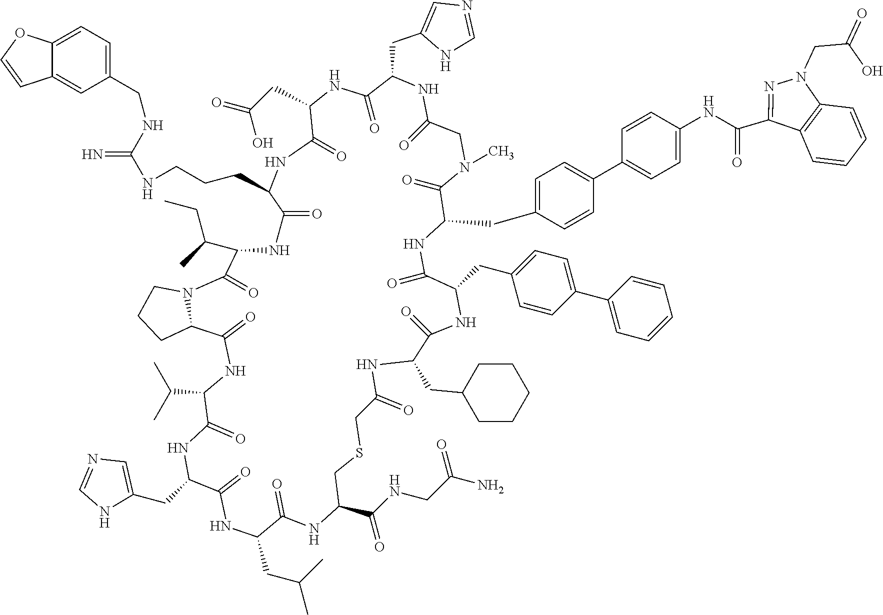 [SMILES: CC[C@H](C)[C@@H]1NC(=O)[C@@H](CCCNC(=N)NCc2ccc3occc3c2)NC(=O)[C@H](CC(=O)O)NC(=O)[C@H](Cc2cnc[nH]2)NC(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 479 was prepared on a 50 umol scale. It was purified by Preparative LC/MS. The yield of the product was 2.7 mg, and its estimated purity by LCMS analysis was 90%. Analysis condition 1: Retention time=1.55 min. ESI-MS(+) m/z [M+2H] 2+: 1102.6.

### Preparation of Example 480

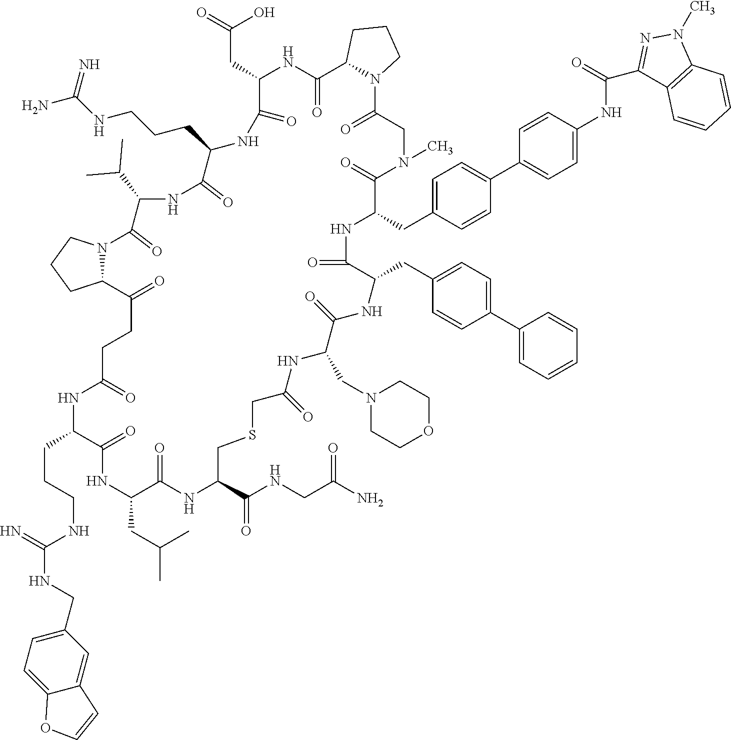 [SMILES: CC(C)C[C@@H]1NC(=O)[C@H](CCCNC(=N)NCc2ccc3occc3c2)NC(=O)CCC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 480 was prepared on a 50 Preparative LC/MS. The yield of the product was 8.4 mg, and its estimated purity by LCMS analysis was 100%. Analysis condition 1: Retention time=1.69 min. ESI-MS(+) m/z [M+2H] 2+: 1064.4.

### Preparation of Example 481

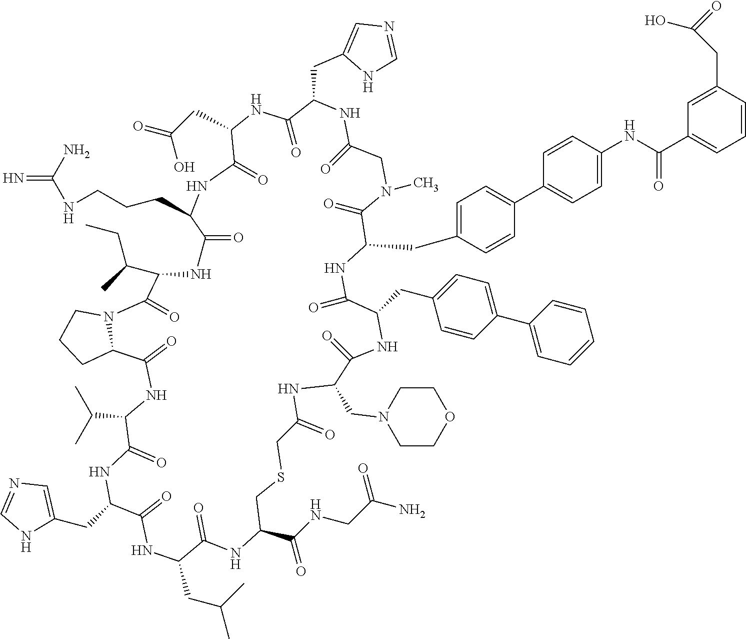 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 481 was prepared on a 50 umol scale. It was purified by Preparative LC/MS. The yield of the product was 1.8 mg, and its estimated purity by LCMS analysis was 96.5%. Analysis condition 2: Retention time=1.31 min. ESI-MS(+) m/z [M+2H] 2+: 1018.8.

### Preparation of Example 482

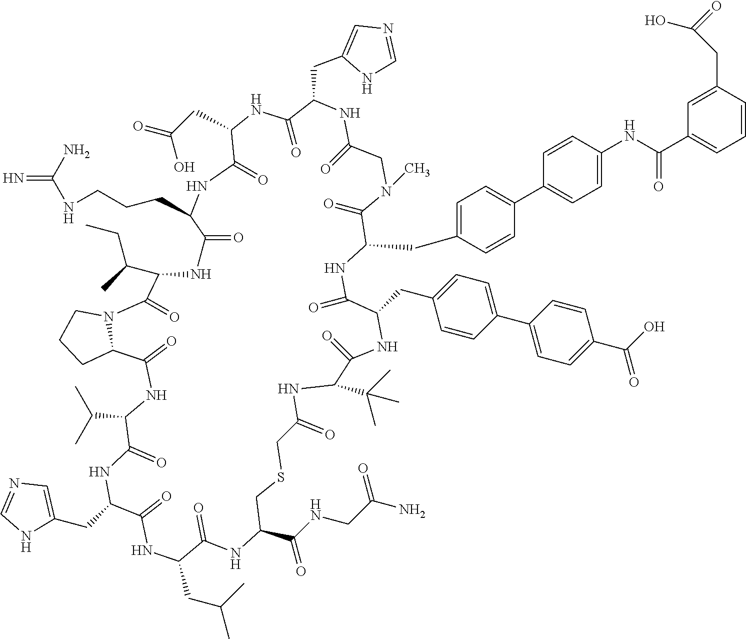 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 482 was prepared on a 50 Preparative LC/MS. The yield of the product was 8.8 mg, and its estimated purity by LCMS analysis was 100%. Analysis condition 1: Retention time=1.03 min. ESI-MS(+) m/z [M+2H] 2+: 1019.2.

### Preparation of Example 483

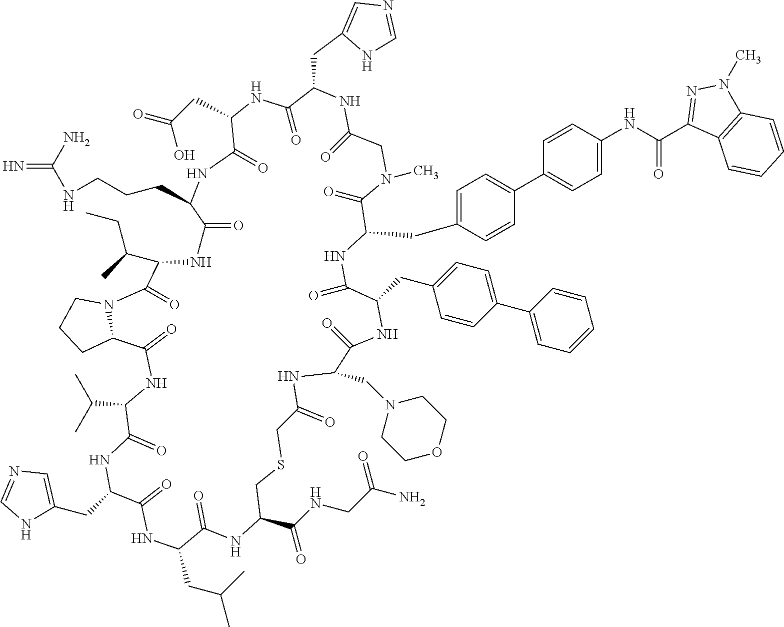 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4nn(C)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 483 was prepared on a 50 umol scale. It was purified by Preparative LC/MS. The yield of the product was 6 mg, and its estimated purity by LCMS analysis was 98.6%. Analysis condition 1: Retention time=1.08 min. ESI-MS(+) m/z [M+2H] 2+: 1041.

### Preparation of Example 484

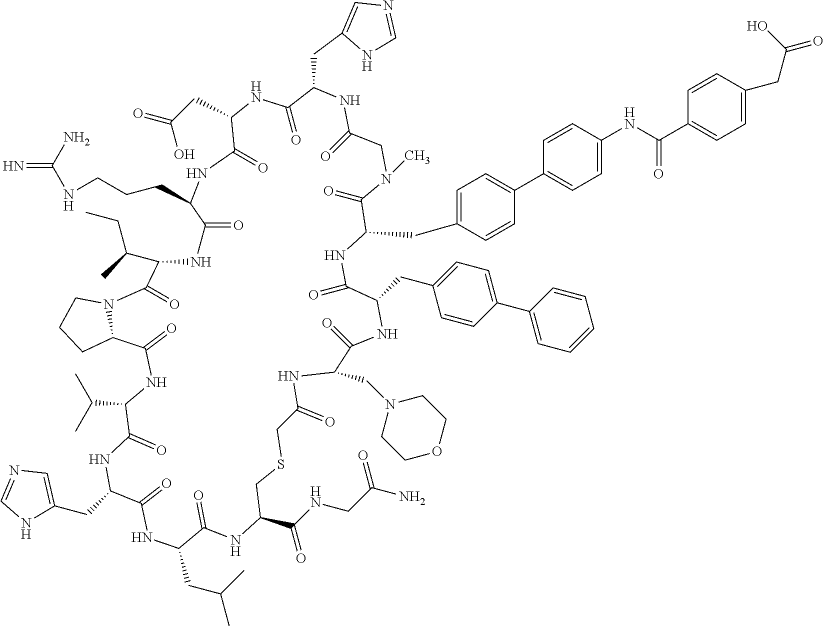 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 484 was prepared on a 50 Preparative LC/MS. The yield of the product was 1.9 mg, and its estimated purity by LCMS analysis was 95.1%. Analysis condition 2: Retention time=1.3 min. ESI-MS(+) m/z [M+3H] 3+: 679.5.

### Preparation of Example 485

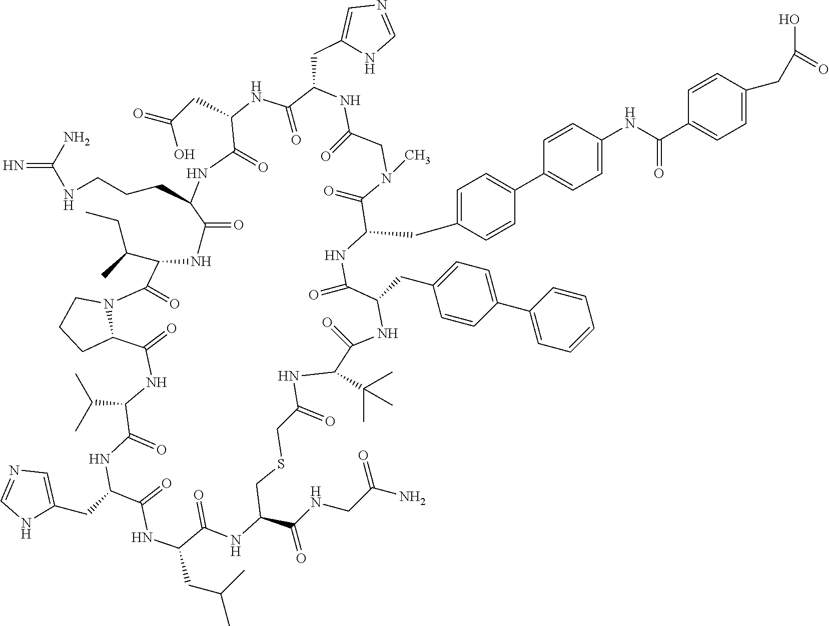 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 485 was prepared on a 50 umol scale. It was purified by Preparative LC/MS. The yield of the product was 20.5 mg, and its estimated purity by LCMS analysis was 91.5%. Analysis condition 2: Retention time=1.34 min. ESI-MS(+) m/z [M+H]⁺: 1992.6.

### Preparation of Example 486

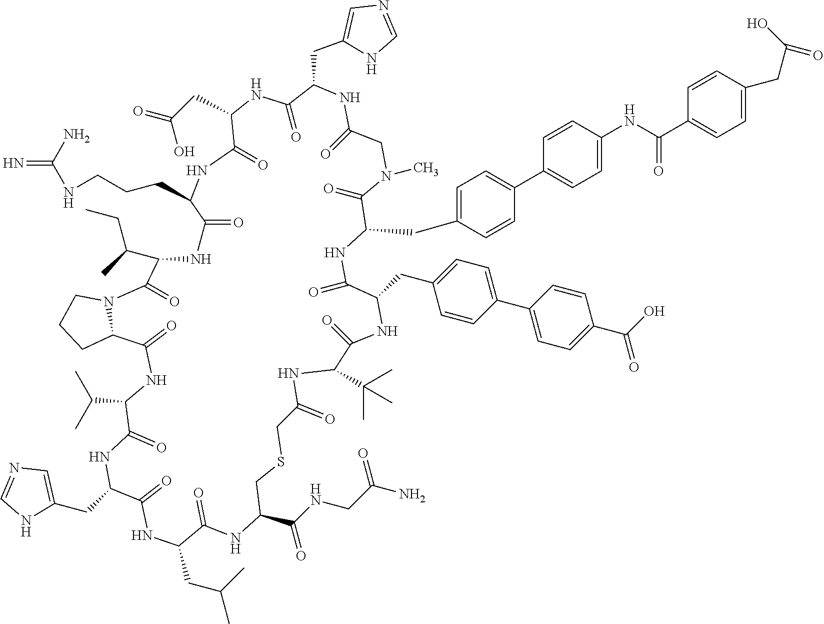 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 486 was prepared on a 50 Preparative LC/MS. The yield of the product was 1.4 mg, and its estimated purity by LCMS analysis was 90.4%. Analysis condition 1: Retention time=1.03 min. ESI-MS(+) m/z [M+2H] 2+: 1019.6.

### Preparation of Example 487

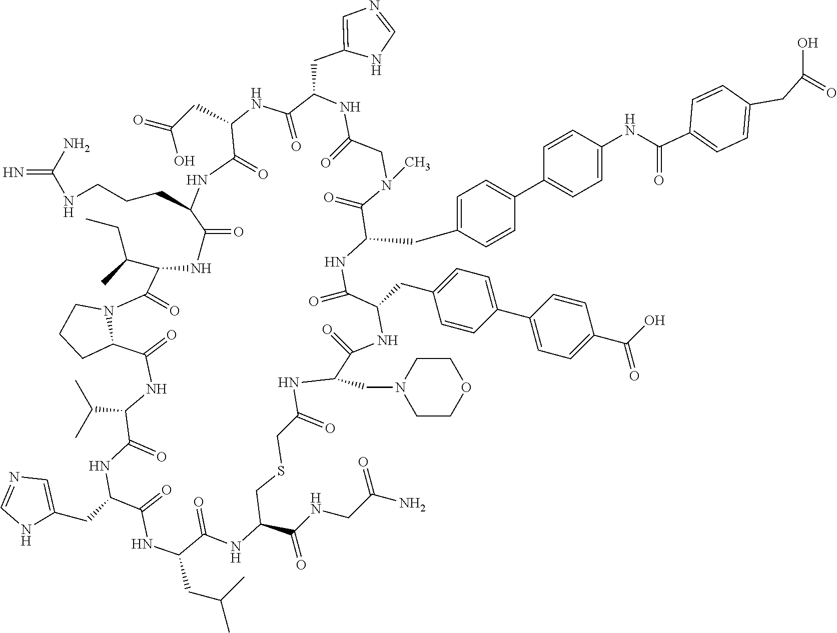 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 487 was prepared on a 50 umol scale. It was purified by Preparative LC/MS. The yield of the product was 5 mg, and its estimated purity by LCMS analysis was 97%. Analysis condition 1: Retention time=1.06 min. ESI-MS(+) m/z [M+2H] 2+: 1041.1.

### Preparation of Example 488

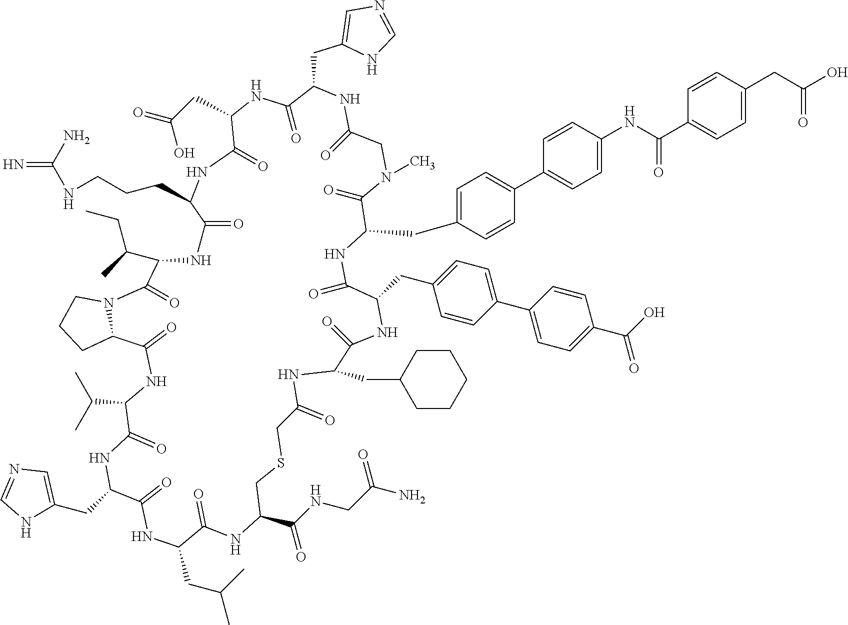 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 488 was prepared on a 50 Preparative LC/MS. The yield of the product was 4.5 mg, and its estimated purity by LCMS analysis was 100%. Analysis condition 1: Retention time=1.09 min. ESI-MS(+) m/z [M+2H] 2+: 1039.2.

### Preparation of Example 489

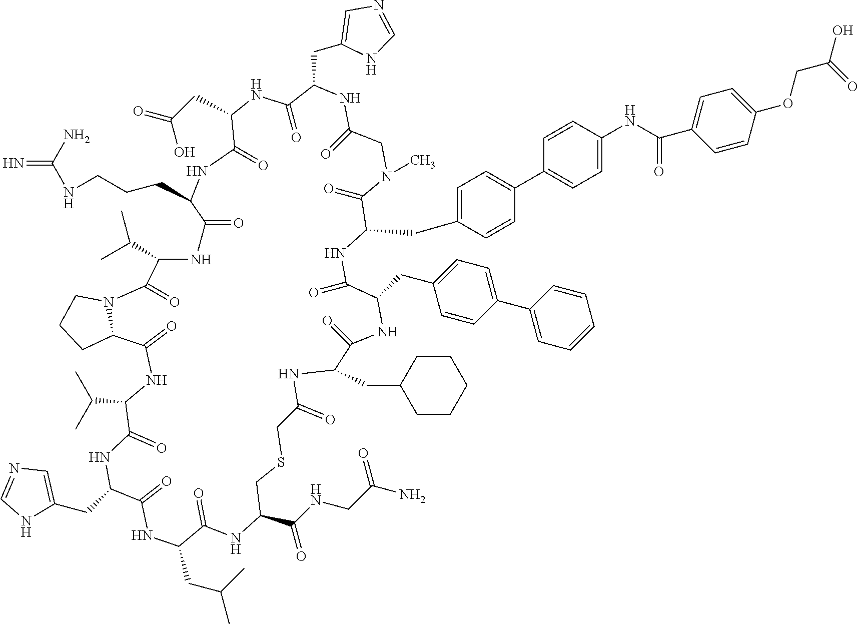 [SMILES: CC(C)C[C@@H]1NC(=O)[C@H](Cc2cnc[nH]2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccc(OCC(=O)O)cc4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC1=O]

Example 489 was prepared on a 50 umol scale. It was purified by Preparative LC/MS. The yield of the product was 10 mg, and its estimated purity by LCMS analysis was 98%. Analysis condition 1: Retention time=1.4 min. ESI-MS(+) m/z [M+2H] 2+: 1018.6.

### Preparation of Example 490

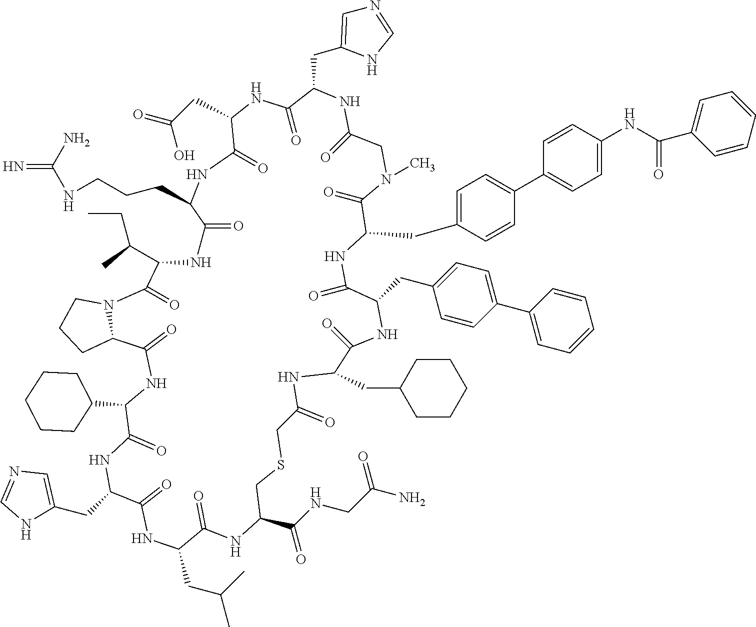 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccccc4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C2CCCCC2)NC(=O)[C@@H]2CCCN2C1=O]

Example 490 was prepared on a 50 Preparative LC/MS. The yield of the product was 27.8 mg, and its estimated purity by LCMS analysis was 90.1%. Analysis condition 2: Retention time=1.59 min. ESI-MS(+) m/z [M+2H] 2+: 1008.4.

### Preparation of Example 491

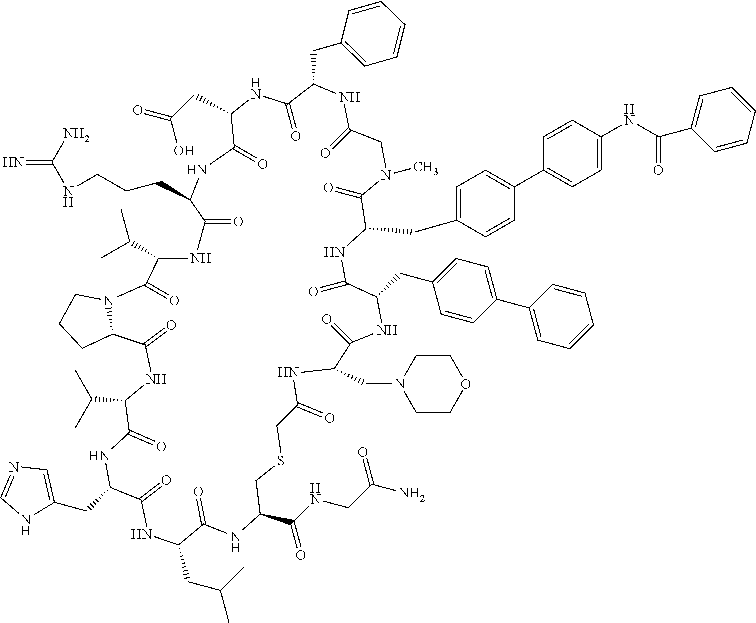 [SMILES: CC(C)C[C@@H]1NC(=O)[C@H](Cc2cnc[nH]2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccc2)NC(=O)CN(C)C(=O)[C@H](Cc2ccc(-c3ccc(NC(=O)c4ccccc4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 491 was prepared on a 50 umol scale. It was purified by Preparative LC/MS. The yield of the product was 20.8 mg, and its estimated purity by LCMS analysis was 93.9%. Analysis condition 2: Retention time=1.55 min. ESI-MS(+) m/z [M+3H] 3+: 658.7.

### Preparation of Example 492

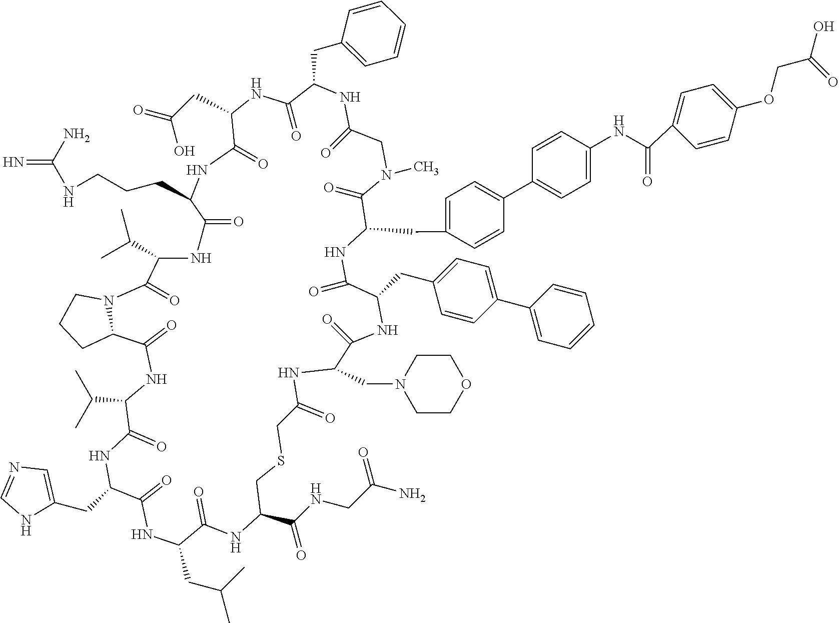 [SMILES: CC(C)C[C@@H]1NC(=O)[C@H](Cc2cnc[nH]2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccc2)NC(=O)CN(C)C(=O)[C@H](Cc2ccc(-c3ccc(NC(=O)c4ccc(OCC(=O)O)cc4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 492 was prepared on a 50 Preparative LC/MS. The yield of the product was 21 mg, and its estimated purity by LCMS analysis was 91%. Analysis condition 2: Retention time=1.49 min. ESI-MS(+) m/z [M+2H] 2+: 1024.8.

### Preparation of Example 493

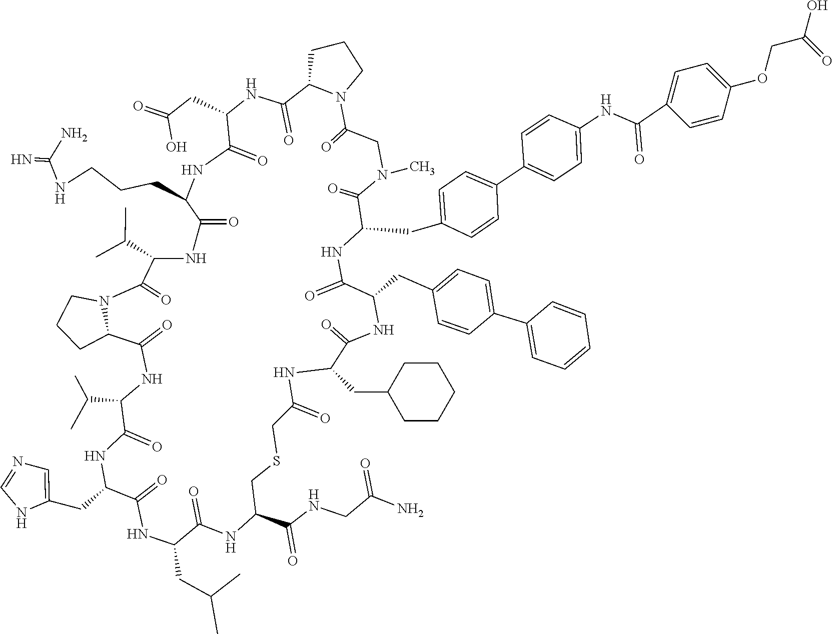 [SMILES: CC(C)C[C@@H]1NC(=O)[C@H](Cc2cnc[nH]2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4ccc(OCC(=O)O)cc4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC1=O]

Example 493 was prepared on a 50 umol scale. It was purified by Preparative LC/MS. The yield of the product was 33.6 mg, and its estimated purity by LCMS analysis was 92.2%. Analysis condition 1: Retention time=1.32 min. ESI-MS(+) m/z [M+H]⁺: 1995.2.

### Preparation of Example 494

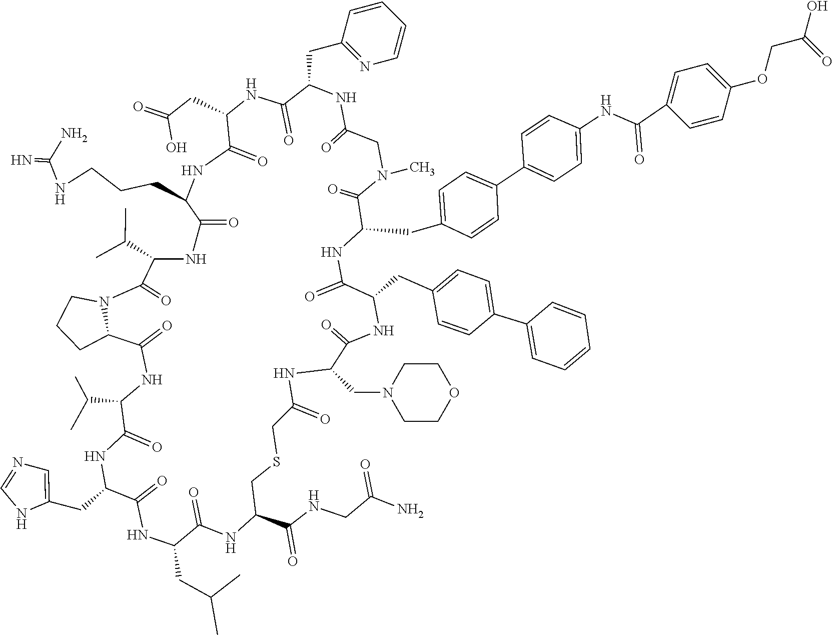 [SMILES: CC(C)C[C@@H]1NC(=O)[C@H](Cc2cnc[nH]2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(=O)c4ccc(OCC(=O)O)cc4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 494 was prepared on a 50 Preparative LC/MS. The yield of the product was 26.2 mg, and its estimated purity by LCMS analysis was 90.9%. Analysis condition 2: Retention time=1.34 min. ESI-MS(+) m/z [M+2H] 2+: 1025.5.

### Preparation of Example 495

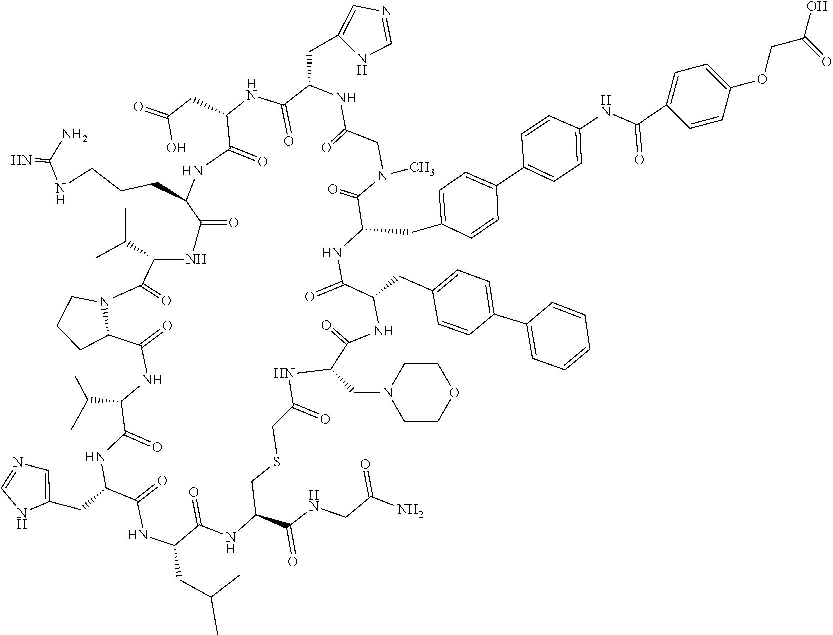 [SMILES: CC(C)C[C@@H]1NC(=O)[C@H](Cc2cnc[nH]2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccc(OCC(=O)O)cc4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 495 was prepared on a 50 umol scale. It was purified by Preparative LC/MS. The yield of the product was 5.4 mg, and its estimated purity by LCMS analysis was 91.8%. Analysis condition 1: Retention time=1.31 min. ESI-MS(+) m/z [M+2H] 2+: 1019.7.

### Preparation of Example 496

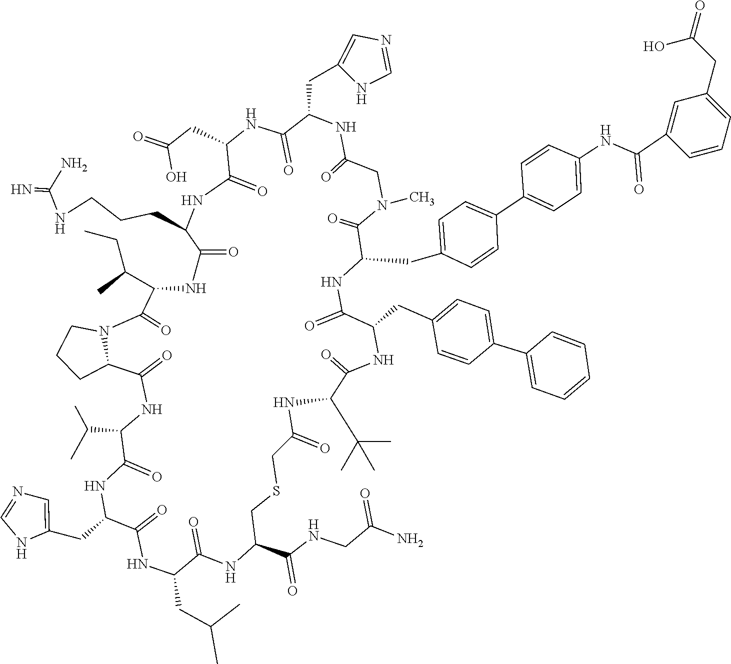 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 496 was prepared on a 50 Preparative LC/MS. The yield of the product was 9.6 mg, and its estimated purity by LCMS analysis was 91.9%. Analysis condition 2: Retention time=1.36 min. ESI-MS(+) m/z [M+H]⁺: 1993.7.

### Preparation of Example 497

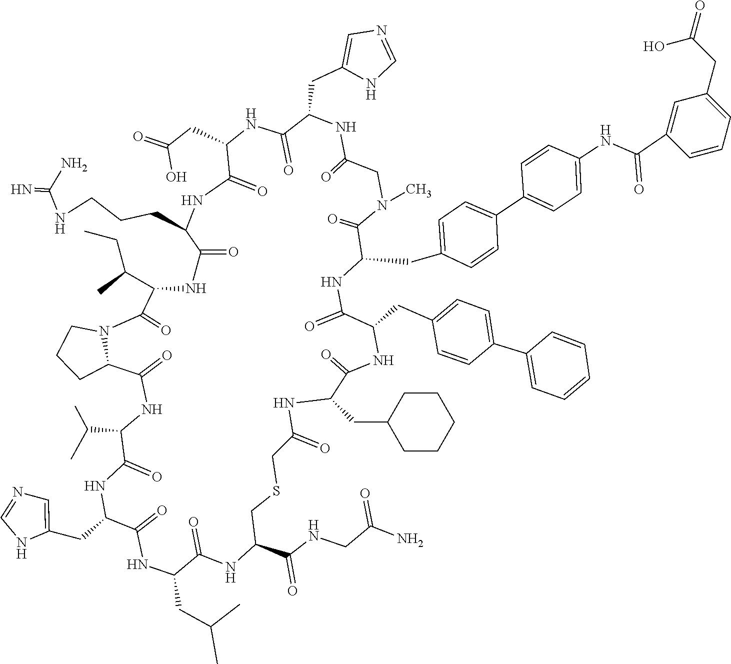 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 497 was prepared on a 50 umol scale. It was purified by Preparative LC/MS. The yield of the product was 8.9 mg, and its estimated purity by LCMS analysis was 97.3%. Analysis condition 1: Retention time=1.18 min. ESI-MS(+) m/z [M+2H] 2+: 1039.

### Preparation of Example 498

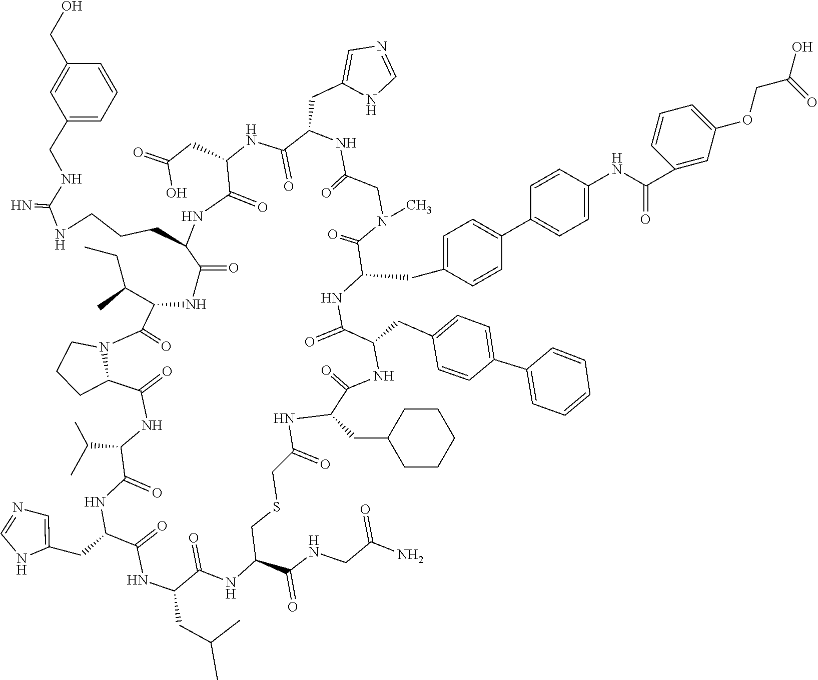 [SMILES: CC[C@H](C)[C@@H]1NC(=O)[C@@H](CCCNC(=N)NCc2cccc(CO)c2)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 498 was prepared on a 50 Preparative LC/MS. The yield of the product was 19.5 mg, and its estimated purity by LCMS analysis was 90.3%. Analysis condition 1: Retention time=1.39 min. ESI-MS(+) m/z [M+2H] 2+: 1085.6.

### Preparation of Example 499

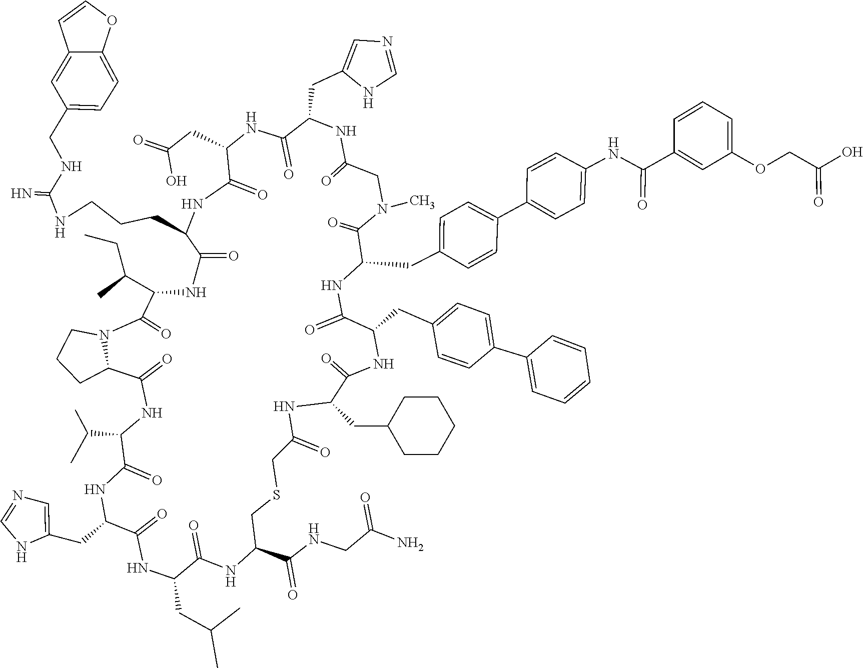 [SMILES: CC[C@H](C)[C@@H]1NC(=O)[C@@H](CCCNC(=N)NCc2ccc3occc3c2)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 499 was prepared on a 50 umol scale. It was purified by Preparative LC/MS. The yield of the product was 2.1 mg, and its estimated purity by LCMS analysis was 91.9%. Analysis condition 2: Retention time=1.54 min. ESI-MS(+) m/z [M+2H] 2+: 1090.3.

### Preparation of Example 500

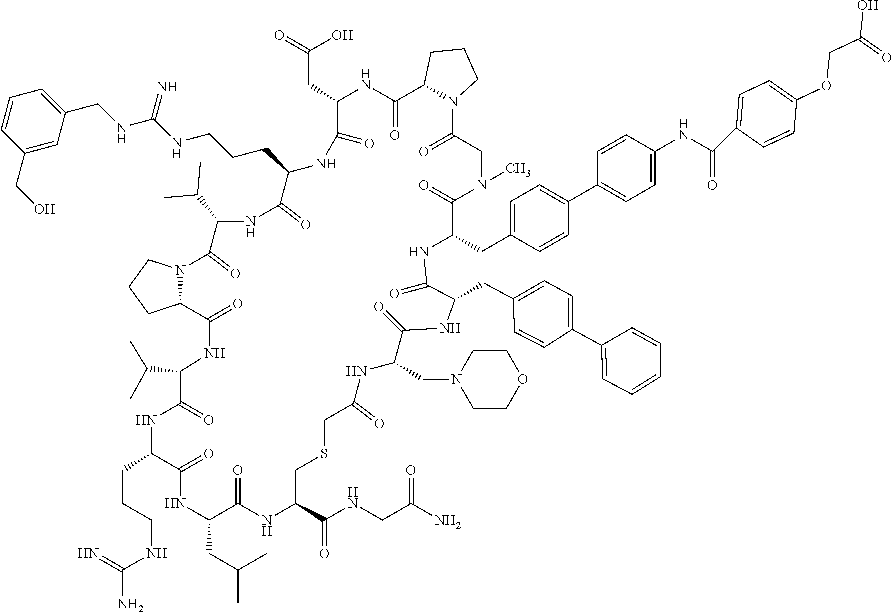 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2cccc(CO)c2)NC(=O)[C@H](CC(=O)O)NC(=O)[C@@H]2CCCN2C(=O)CN(C)C(=O)[C@H](Cc2ccc(-c3ccc(NC(=O)c4ccc(OCC(=O)O)cc4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 500 was prepared on a 50 Preparative LC/MS. The yield of the product was 7.6 mg, and its estimated purity by LCMS analysis was 100%. Analysis condition 1: Retention time=1.37 min. ESI-MS(+) m/z [M+2H] 2+: 1069.5.

### Preparation of Example 501

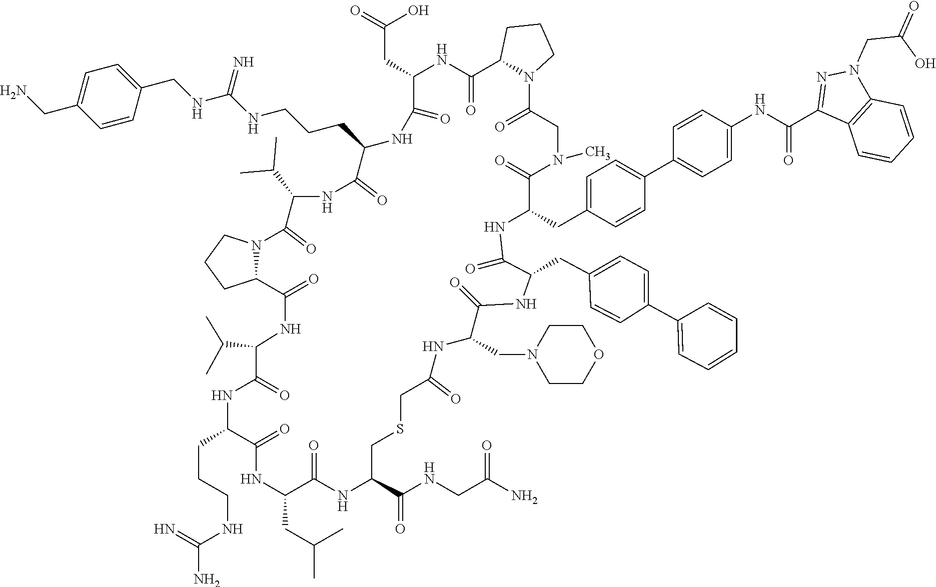 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2ccc(CN)cc2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 501 was prepared on a 50 umol scale. It was purified by Preparative LC/MS. The yield of the product was 4.7 mg, and its estimated purity by LCMS analysis was 98.2%. Analysis condition 2: Retention time=1.35 min. ESI-MS(+) m/z [M+2H] 2+: 1081.1.

### Preparation of Example 502

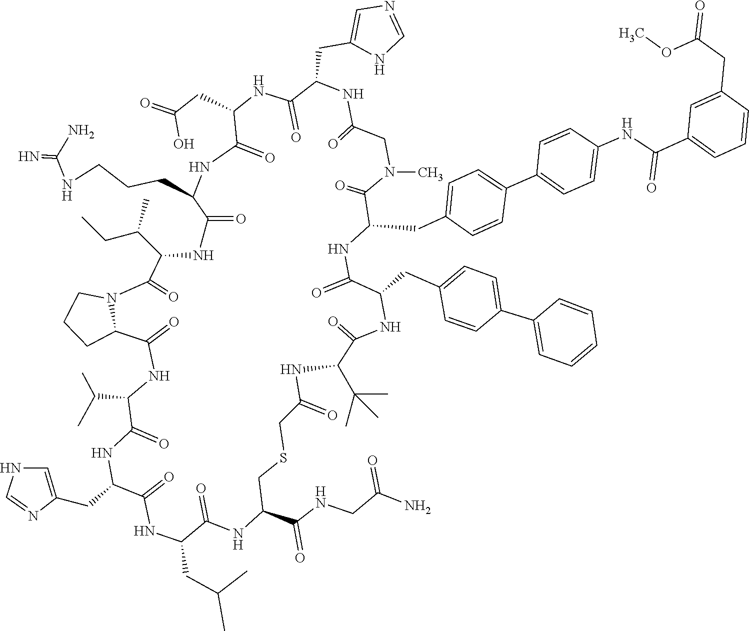 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(CC(=O)OC)c4)cc3)cc2)NC(=O)[C@H](Cc2ccc(-c3ccccc3)cc2)NC(=O)[C@H](C(C)(C)C)NC(=O)CSC[C@@H](C(=O)NCC(N)=O)NC(=O)[C@H](CC(C)C)NC(=O)[C@H](Cc2c[nH]cn2)NC(=O)[C@H](C(C)C)NC(=O)[C@@H]2CCCN2C1=O]

Example 502 was prepared on a 50 Preparative LC/MS. The yield of the product was 4.8 mg, and its estimated purity by LCMS analysis was 99.1%. Analysis condition 1: Retention time=1.52 min. ESI-MS(+) m/z [M+2H]²⁺: 1004.1.

### Preparation of Example 503

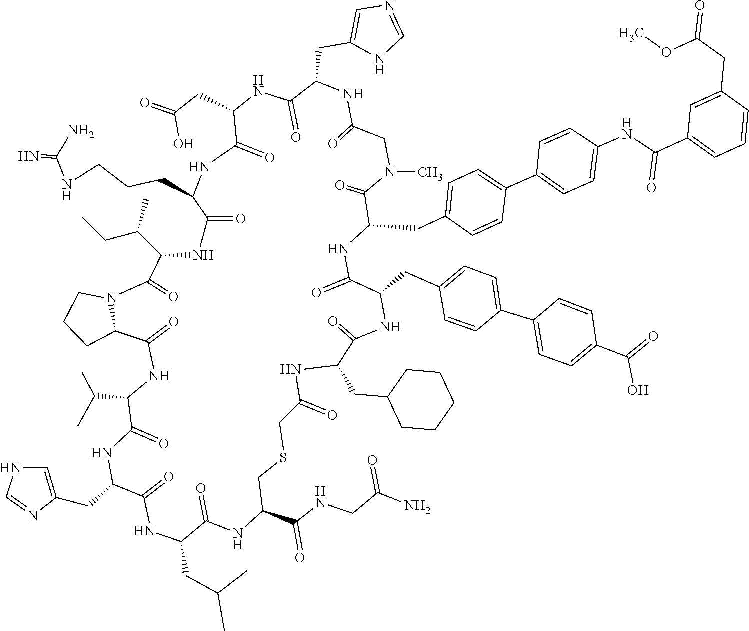 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(CC(=O)OC)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2c[nH]cn2)NC(=O)[C@H](C(C)C)NC(=O)[C@@H]2CCCN2C1=O]

Example 503 was prepared on a 50 umol scale. It was purified by Preparative LC/MS. The yield of the product was 1.3 mg, and its estimated purity by LCMS analysis was 97.7%. Analysis condition 2: Retention time=1.45 min. ESI-MS(+) m/z [M+2H]²⁺: 1046.7.

### Preparation of Example 504

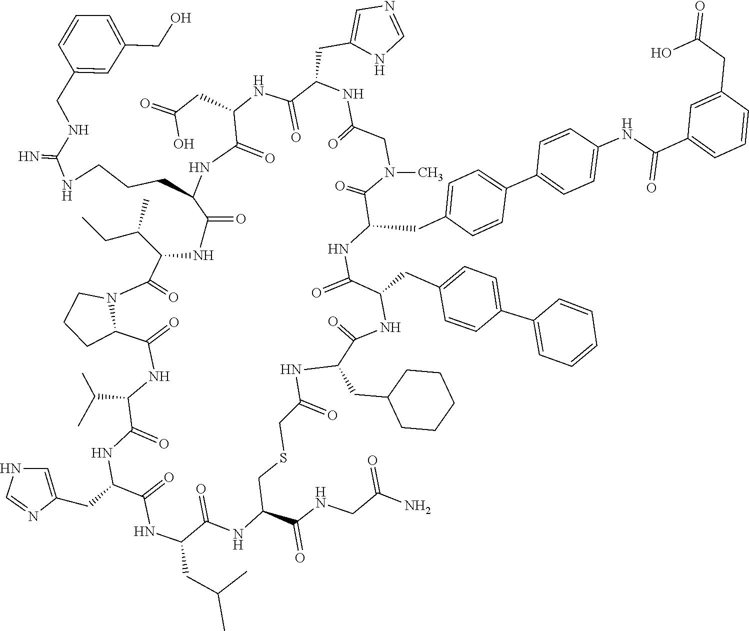 [SMILES: CC[C@H](C)[C@@H]1NC(=O)[C@@H](CCCNC(=N)NCc2cccc(CO)c2)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2c[nH]cn2)NC(=O)[C@H](C(C)C)NC(=O)[C@@H]2CCCN2C1=O]

Example 504 was prepared on a 50 Preparative LC/MS. The yield of the product was 11.9 mg, and its estimated purity by LCMS analysis was 97.5%. Analysis condition 1: Retention time=1.44 min. ESI-MS(+) m/z [M+2H]²⁺: 1077.4.

### Preparation of Example 505

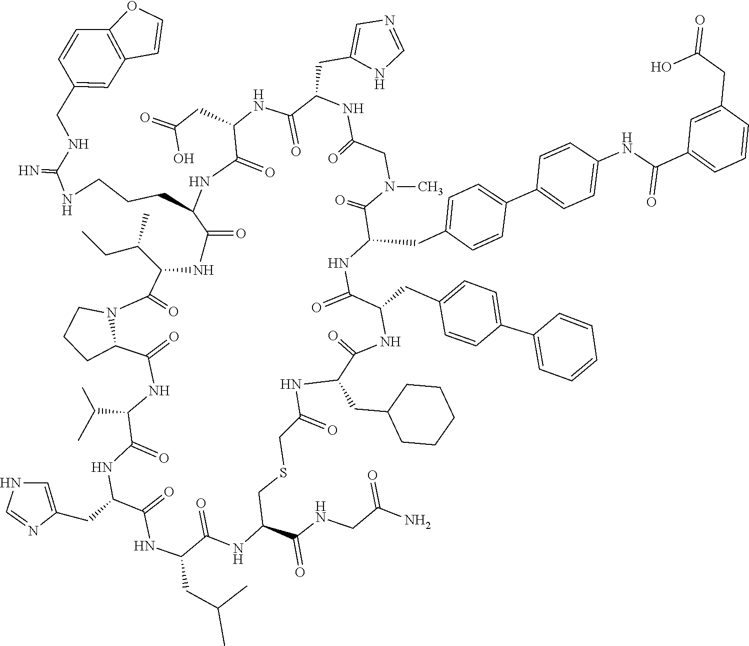 [SMILES: CC[C@H](C)[C@@H]1NC(=O)[C@@H](CCCNC(=N)NCc2ccc3occc3c2)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2c[nH]cn2)NC(=O)[C@H](C(C)C)NC(=O)[C@@H]2CCCN2C1=O]

Example 505 was prepared on a 50 umol scale. It was purified by Preparative LC/MS. The yield of the product was 7.9 mg, and its estimated purity by LCMS analysis was 100%. Analysis condition 1: Retention time=1.53 min. ESI-MS(+) m/z [M+2H]²⁺: 1082.3.

### Preparation of Example 506

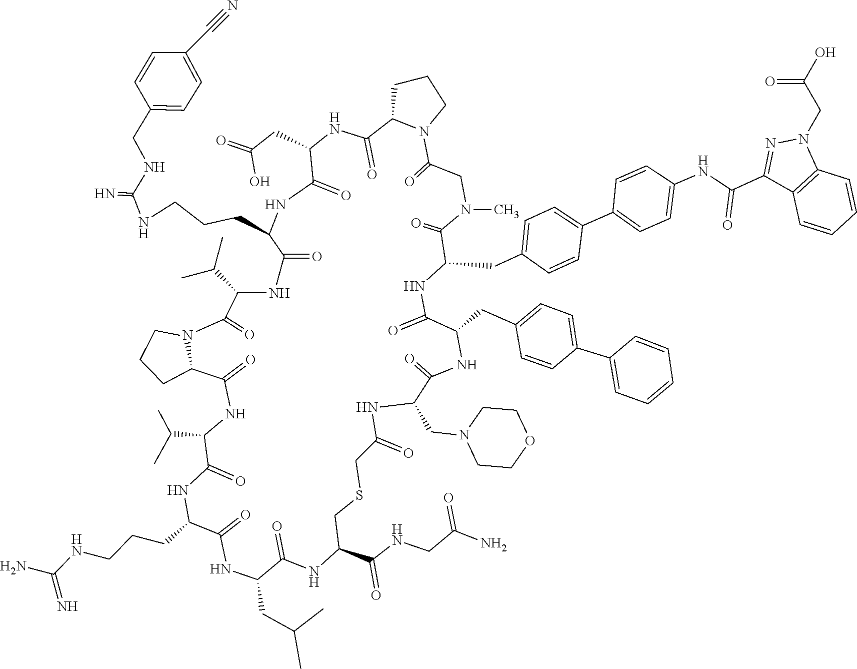 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2ccc(C#N)cc2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 506 was prepared on a 50 Preparative LC/MS. The yield of the product was 1.9 mg, and its estimated purity by LCMS analysis was 100%. Analysis condition 1: Retention time=1.52 min. ESI-MS(+) m/z [M+2H]²⁺: 1079.

### Preparation of Example 507

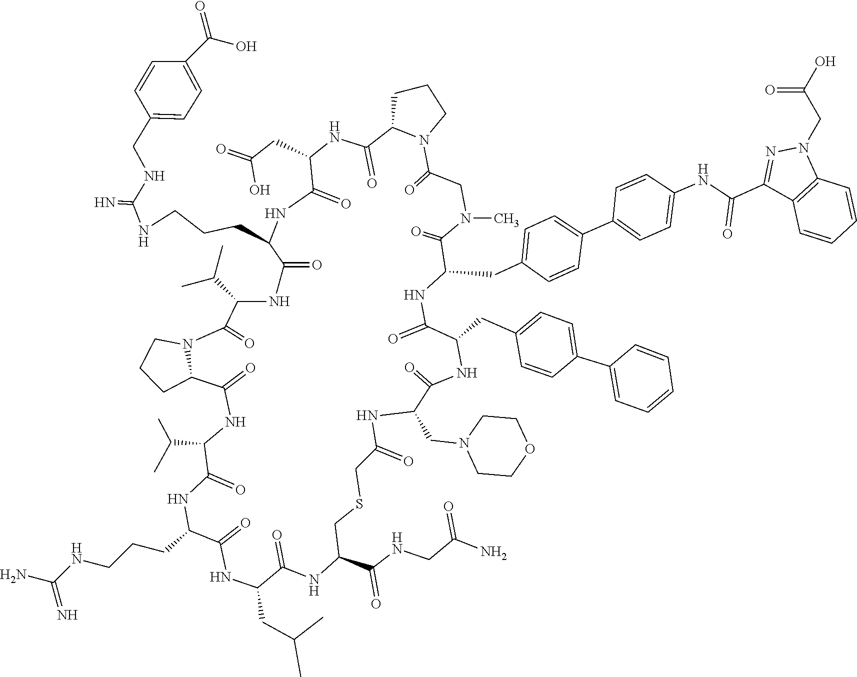 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2ccc(C(=O)O)cc2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 507 was prepared on a 50 umol scale. It was purified by Preparative LC/MS. The yield of the product was 8.8 mg, and its estimated purity by LCMS analysis was 100%. Analysis condition 2: Retention time=1.5 min. ESI-MS(+) m/z [M+3H]³⁺: 725.9.

### Preparation of Example 508

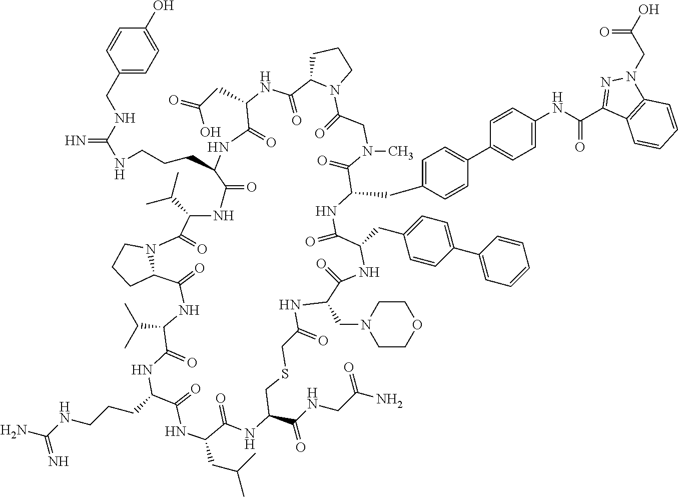 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2ccc(O)cc2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 508 was prepared on a 50 Preparative LC/MS. The yield of the product was 15.3 mg, and its estimated purity by LCMS analysis was 98.3%. Analysis condition 1: Retention time=1.08 min. ESI-MS(+) m/z [M+2H]²⁺: 1074.3.

### Preparation of Example 509

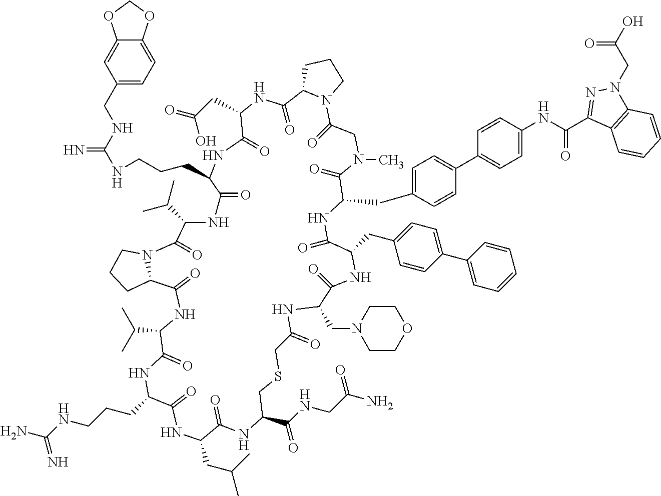 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2ccc3c(c2)OCO3)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 509 was prepared on a 50 umol scale. It was purified by Preparative LC/MS. The yield of the product was 1.6 mg, and its estimated purity by LCMS analysis was 100%. Analysis condition 1: Retention time=1.56 min. ESI-MS(+) m/z [M+2H]²⁺: 1088.6.

### Preparation of Example 510

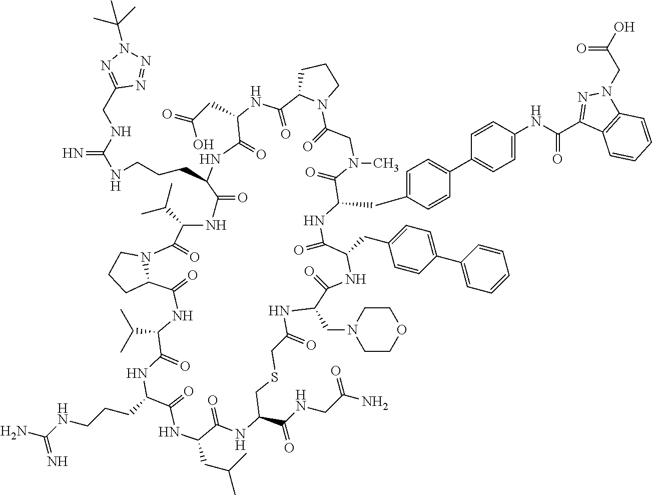 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2nnn(C(C)(C)C)n2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 510 was prepared on a 50 Preparative LC/MS. The yield of the product was 8.2 mg, and its estimated purity by LCMS analysis was 100%. Analysis condition 2: Retention time=1.56 min. ESI-MS(+) m/z [M+2H]²⁺: 1090.4.

### Preparation of Example 511

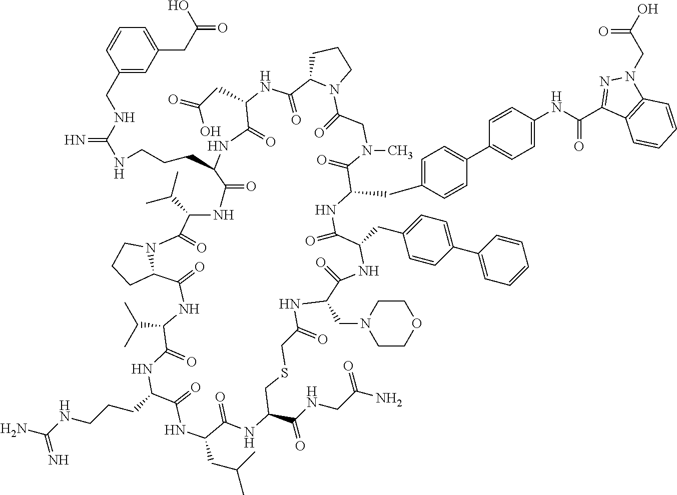 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2cccc(CC(=O)O)c2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 511 was prepared on a 50 umol scale. It was purified by Preparative LC/MS. The yield of the product was 4.9 mg, and its estimated purity by LCMS analysis was 92.7%. Analysis condition 1: Retention time=1.26 min. ESI-MS(+) m/z [M+2H]²⁺: 1095.8.

### Preparation of Example 512

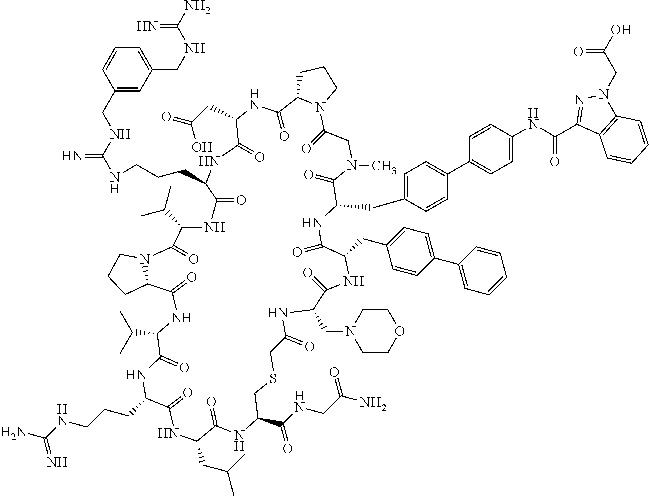 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2cccc(CNC(=N)N)c2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 512 was prepared on a 50 Preparative LC/MS. The yield of the product was 2.9 mg, and its estimated purity by LCMS analysis was 87.4%. Analysis condition 1: Retention time=1.35 min. ESI-MS(+) m/z [M+2H]²⁺: 1102.

### Preparation of Example 513

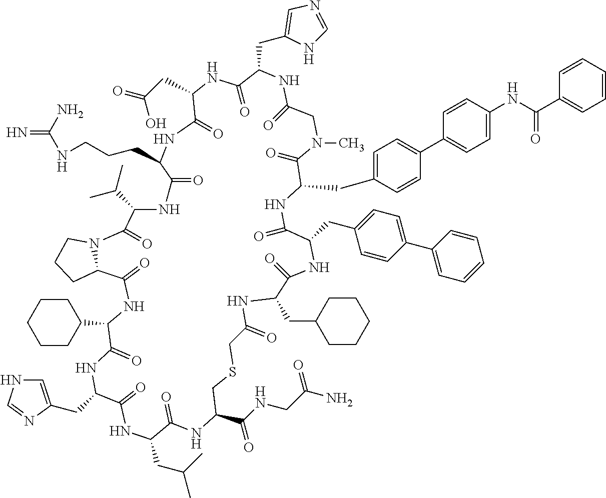 [SMILES: CC(C)C[C@@H]1NC(=O)[C@H](Cc2c[nH]cn2)NC(=O)[C@H](C2CCCCC2)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccccc4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC1=O]

Example 513 was prepared on a 50 umol scale. It was purified by Preparative LC/MS. The yield of the product was 16.2 mg, and its estimated purity by LCMS analysis was 100%. Analysis condition 1: Retention time=1.63 min. ESI-MS(+) m/z [M+2H]²⁺: 1001.3.

### Preparation of Example 514

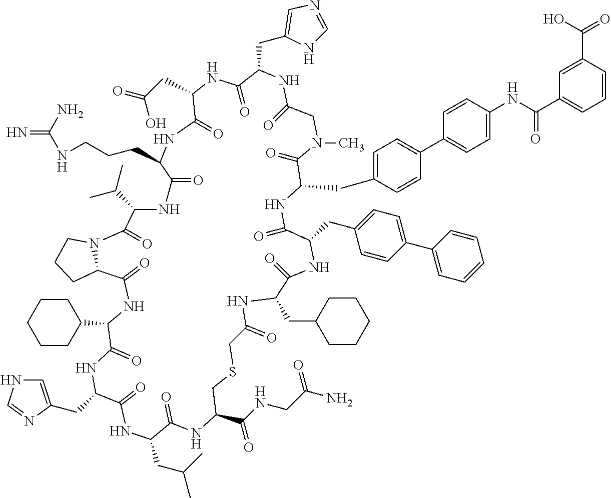 [SMILES: CC(C)C[C@@H]1NC(=O)[C@H](Cc2c[nH]cn2)NC(=O)[C@H](C2CCCCC2)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(C(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC1=O]

Example 514 was prepared on a 50 umol scale. It was purified by Preparative LC/MS. The yield of the product was 23.2 mg, and its estimated purity by LCMS analysis was 100%. Analysis condition 2: Retention time=1.5 min. ESI-MS(+) m/z [M+2H]²⁺: 1023.5.

### Preparation of Example 515

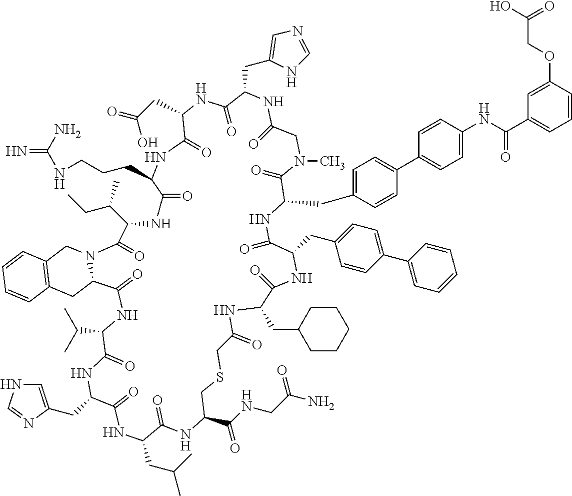 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2c[nH]cn2)NC(=O)[C@H](C(C)C)NC(=O)[C@@H]2Cc3ccccc3CN2C1=O]

Example 515 was prepared on a 50 umol scale. It was purified by Preparative LC/MS. The yield of the product was 17.8 mg, and its estimated purity by LCMS analysis was 90.3%. Analysis condition 2: Retention time=1.55 min. ESI-MS(+) m/z [M+2H]²⁺: 1056.3.

### Preparation of Example 516

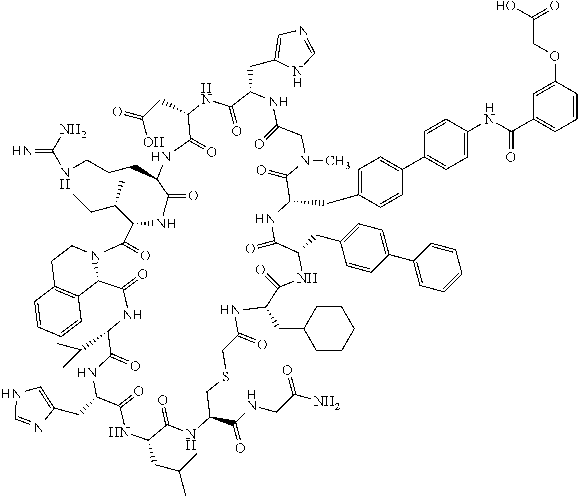 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2c[nH]cn2)NC(=O)[C@H](C(C)C)NC(=O)[C@@H]2c3ccccc3CCN2C1=O]

Example 516 was prepared on a 50 Preparative LC/MS. The yield of the product was 16.4 mg, and its estimated purity by LCMS analysis was 96.3%. Analysis condition 1: Retention time=1.49 min. ESI-MS(+) m/z [M+2H]²⁺: 1056.6.

### Preparation of Example 517

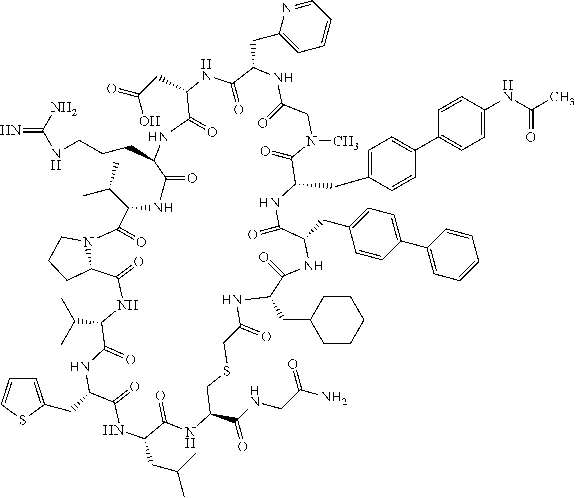

Example 517 was prepared on a 90050 umol scale. It was purified by Preparative LC/MS. The yield of the product was 34.7 mg, and its estimated purity by LCMS analysis was 90.7%. Analysis condition 1: Retention time=1.56 min. ESI-MS(+) m/z [M+H]⁺: 1928.7.

### Preparation of Example 518

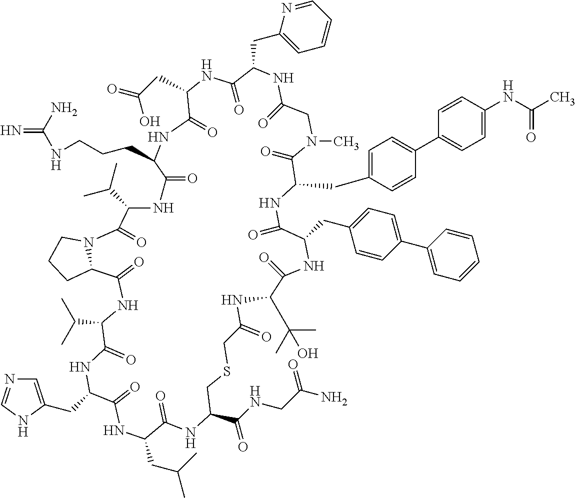 [SMILES: CC(=O)Nc1ccc(-c2ccc(C[C@@H]3NC(=O)[C@H](Cc4ccc(-c5ccccc5)cc4)NC(=O)[C@H](C(C)(C)O)NC(=O)CSC[C@@H](C(=O)NCC(N)=O)NC(=O)[C@H](CC(C)C)NC(=O)[C@H](Cc4cnc[nH]4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 518 was prepared on a 50 umol scale. It was purified by Preparative LC/MS. The yield of the product was 16.6 mg, and its estimated purity by LCMS analysis was 97.4%. Analysis condition 1: Retention time=1.43 min. ESI-MS(+) m/z [M+H]⁺: 1872.

### Preparation of Example 519

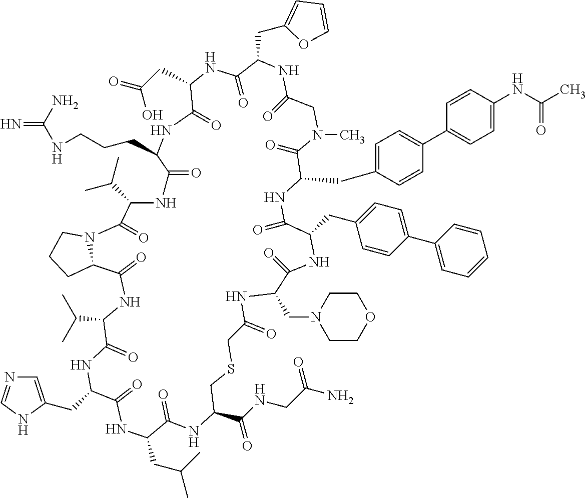 [SMILES: CC(=O)Nc1ccc(-c2ccc(C[C@@H]3NC(=O)[C@H](Cc4ccc(-c5ccccc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cnc[nH]4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccco4)NC(=O)CN(C)C3=O)cc2)cc1]

Example 519 was prepared on a 50 umol scale. It was purified by Preparative LC/MS. The yield of the product was 25.7 mg, and its estimated purity by LCMS analysis was 100%. Analysis condition 1: Retention time=1.31 min. ESI-MS(+) m/z [M+2H]²⁺: 952.1.

### Preparation of Example 520

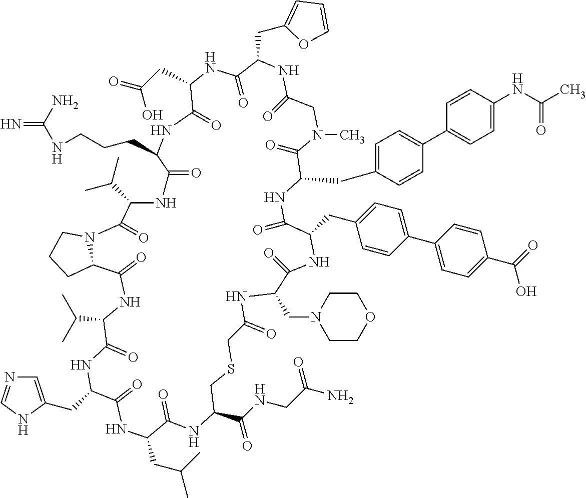 [SMILES: CC(=O)Nc1ccc(-c2ccc(C[C@@H]3NC(=O)[C@H](Cc4ccc(-c5ccc(C(=O)O)cc5)cc4)NC(=O)[C@H](CN4CCOCC4)NC(=O)CSC[C@@H](C(=O)NCC(N)=O)NC(=O)[C@H](CC(C)C)NC(=O)[C@H](Cc4cnc[nH]4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccco4)NC(=O)CN(C)C3=O)cc2)cc1]

Example 520 was prepared on a 50 Preparative LC/MS. The yield of the product was 11.6 mg, and its estimated purity by LCMS analysis was 96.9%. Analysis condition 2: Retention time=1.18 min. ESI-MS(+) m/z [M+2H]²⁺: 973.7.

### Preparation of Example 521

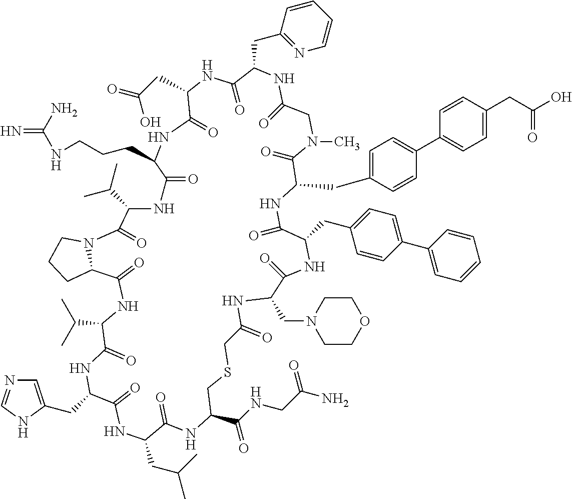 [SMILES: CC(C)C[C@@H]1NC(=O)[C@H](Cc2cnc[nH]2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(CC(=O)O)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 521 was prepared on a 50 umol scale. It was purified by Preparative LC/MS. The yield of the product was 6 mg, and its estimated purity by LCMS analysis was 94.9%. Analysis condition 2: Retention time=1.31 min. ESI-MS(+) m/z [M+2H]²⁺: 1017.3.

### Preparation of Example 522

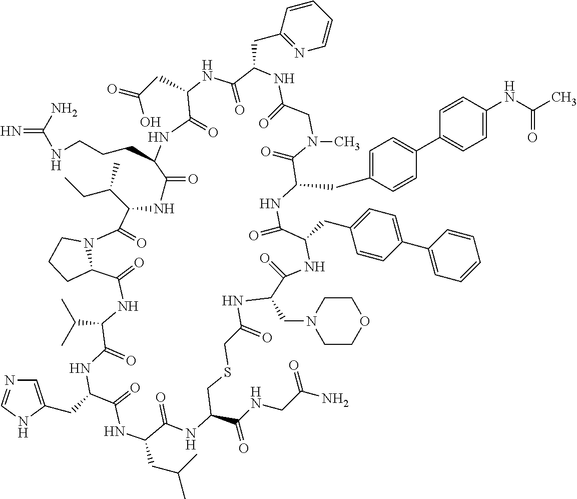 [SMILES: CC[C@H](C)[C@@H]1NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(C)=O)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 522 was prepared on a 50 umol scale. It was purified by Preparative LC/MS. The yield of the product was 32.2 mg, and its estimated purity by LCMS analysis was 96.7%. Analysis condition 2: Retention time=1.27 min. ESI-MS(+) m/z [M+H]⁺: 1926.7.

### Preparation of Example 523

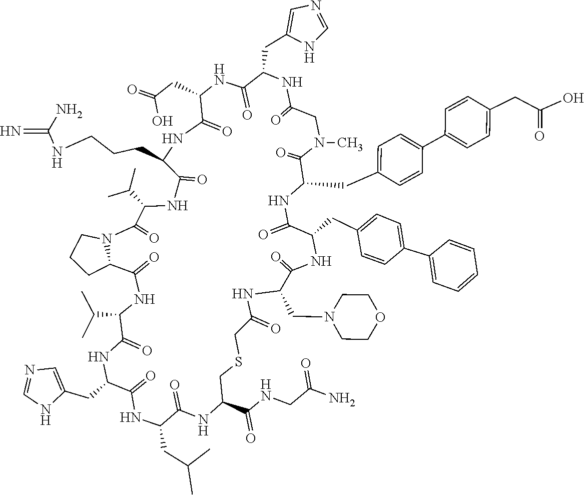 [SMILES: CC(C)C[C@@H]1NC(=O)[C@H](Cc2cnc[nH]2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(CC(=O)O)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 523 was prepared on a 50 umol scale. It was purified by Preparative LC/MS. The yield of the product was 6.7 mg, and its estimated purity by LCMS analysis was 100%. Analysis condition 1: Retention time=1.29 min. ESI-MS(+) m/z [M+2H]²⁺: 1011.9.

### Preparation of Example 524

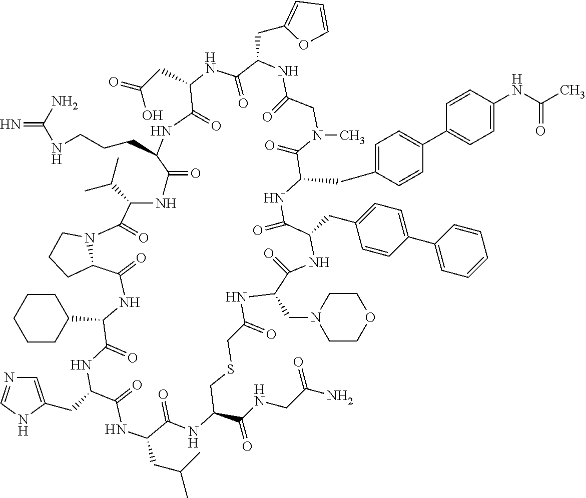

Example 524 was prepared on a 50 umol scale. It was purified by Preparative LC/MS. The yield of the product was 26.7 mg, and its estimated purity by LCMS analysis was 95.7%. Analysis condition 1: Retention time=1.53 min. ESI-MS(+) m/z [M+H]⁺: 1942.

### Preparation of Example 525

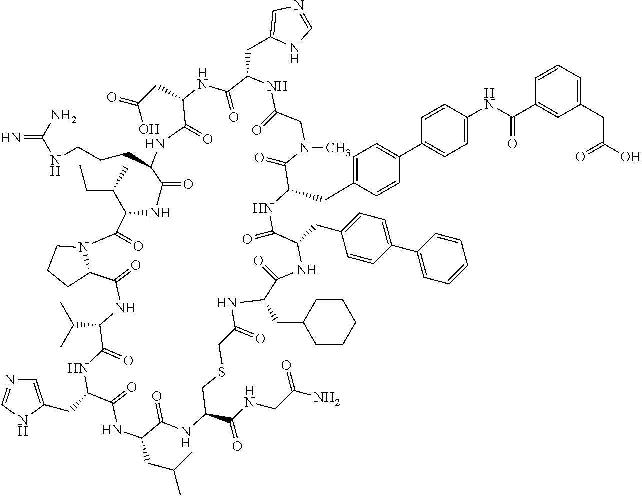 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 525 was prepared on a 50 umol scale. It was purified by Preparative LC/MS. The yield of the product was 16 mg, and its estimated purity by LCMS analysis was 95.1%. Analysis condition 1: Retention time=1.38 min. ESI-MS(+) m/z [M+2H]²⁺: 1010.3.

### Preparation of Example 526

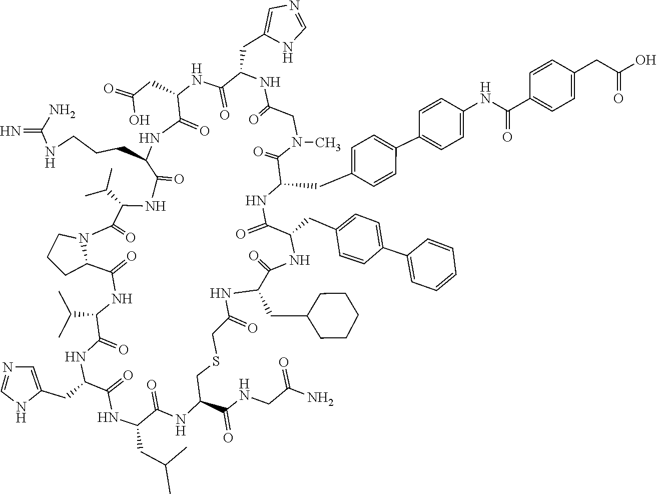 [SMILES: CC(C)C[C@@H]1NC(=O)[C@H](Cc2cnc[nH]2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC1=O]

Example 526 was prepared on a 50 Preparative LC/MS. The yield of the product was 19.4 mg, and its estimated purity by LCMS analysis was 96.4%. Analysis condition 2: Retention time=1.44 min. ESI-MS(+) m/z [M+2H]²⁺: 1010.2.

### Preparation of Example 527

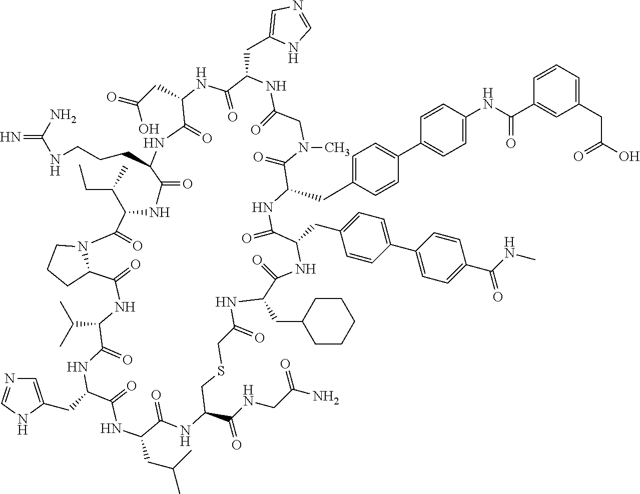 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccc(C(=O)NC)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 527 was prepared on a 50 umol scale. It was purified by Preparative LC/MS. The yield of the product was 21.8 mg, and its estimated purity by LCMS analysis was 95.5%. Analysis condition 1: Retention time=1.23 min. ESI-MS(+) m/z [M+2H]²⁺: 1046.2.

### Preparation of Example 528

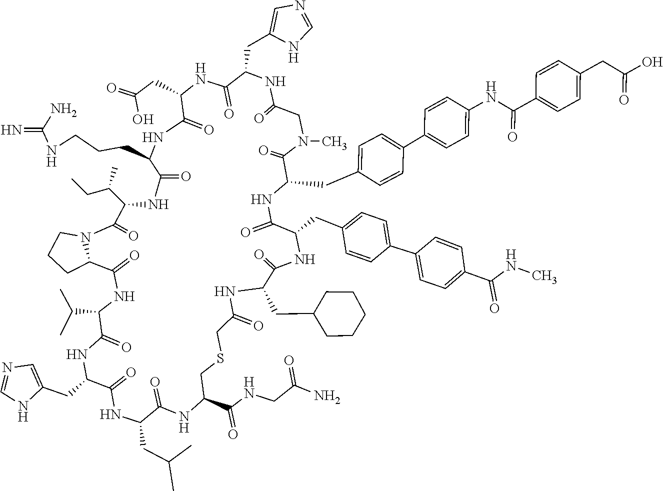 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccc(C(=O)NC)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 528 was prepared on a 50 Preparative LC/MS. The yield of the product was 21 mg, and its estimated purity by LCMS analysis was 94.5%. Analysis condition 2: Retention time=1.27 min. ESI-MS(+) m/z [M+2H]²⁺: 1046.2.

### Preparation of Example 529

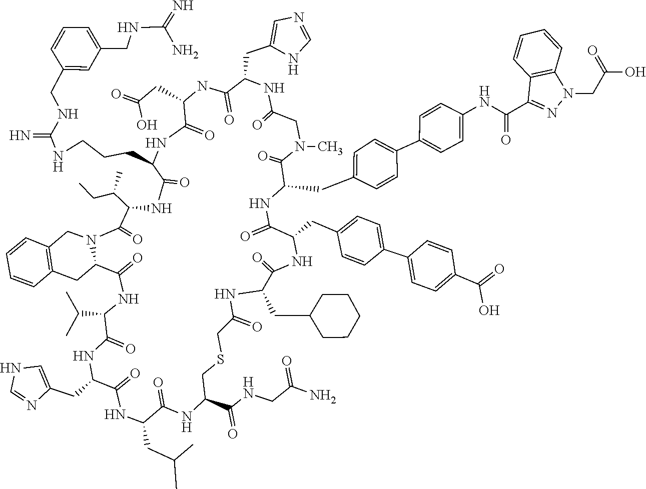

Example 529 was prepared on a 100 umol scale. It was purified by Preparative LC/MS. The yield of the product was 14.6 mg, and its estimated purity by LCMS analysis was 100%. Analysis condition 1: Retention time=1.51 min. ESI-MS(+) m/z [M+2H]²⁺: 1171.2.

### Preparation of Example 530

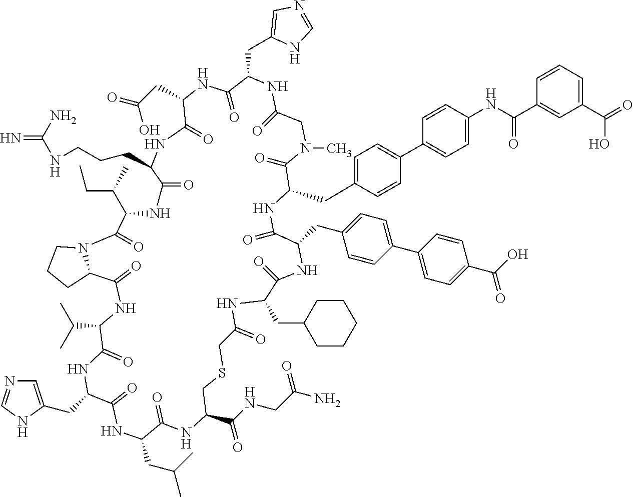 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(C(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 530 was prepared on a 50 umol scale. It was purified by Preparative LC/MS. The yield of the product was 10.6 mg, and its estimated purity by LCMS analysis was 100%. Analysis condition 1: Retention time=1.11 min. ESI-MS(+) m/z [M+2H]²⁺: 1032.1.

### Preparation of Example 531

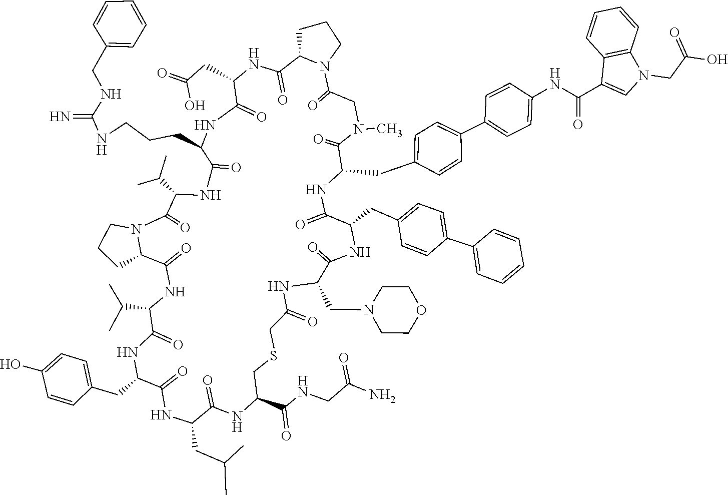 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)NCc2ccccc2)NC(=O)[C@H](CC(=O)O)NC(=O)[C@@H]2CCCN2C(=O)CN(C)C(=O)[C@H](Cc2ccc(-c3ccc(NC(=O)c4cn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 531 was prepared on a 50 umol scale. It was purified by Preparative LC/MS. The yield of the product was 6 mg, and its estimated purity by LCMS analysis was 99.1%. Analysis condition 1: Retention time=1.44 min. ESI-MS(+) m/z [M+2H]²⁺: 1069.8.

### Preparation of Example 532

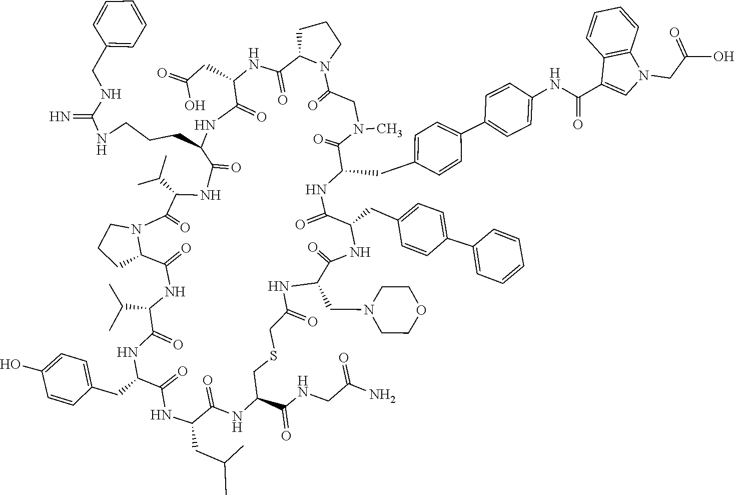 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)NCc2ccccc2)NC(=O)[C@H](CC(=O)O)NC(=O)[C@@H]2CCCN2C(=O)CN(C)C(=O)[C@H](Cc2ccc(-c3ccc(NC(=O)c4cn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 532 was prepared on a 50 Preparative LC/MS. The yield of the product was 6.4 mg, and its estimated purity by LCMS analysis was 96.5%. Analysis condition 1: Retention time=1.53 min. ESI-MS(+) m/z [M+2H]²⁺: 1069.8.

### Preparation of Example 533

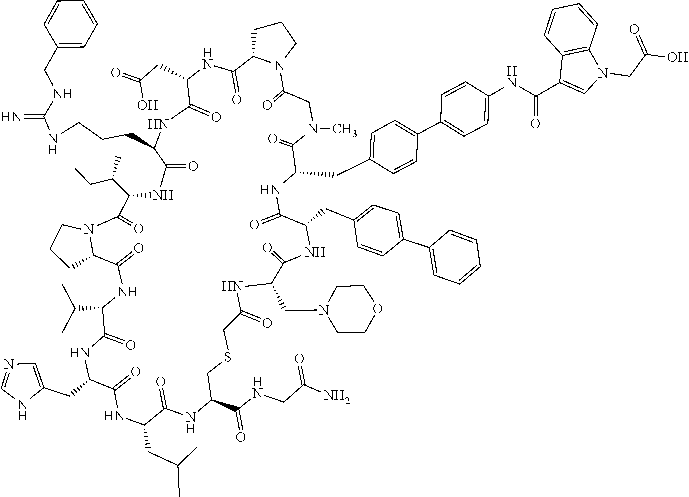 [SMILES: CC[C@H](C)[C@@H]1NC(=O)[C@@H](CCCNC(=N)NCc2ccccc2)NC(=O)[C@H](CC(=O)O)NC(=O)[C@@H]2CCCN2C(=O)CN(C)C(=O)[C@H](Cc2ccc(-c3ccc(NC(=O)c4cn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 533 was prepared on a 50 umol scale. It was purified by Preparative LC/MS. The yield of the product was 5.2 mg, and its estimated purity by LCMS analysis was 100%. Analysis condition 1: Retention time=1.52 min. ESI-MS(+) m/z [M+2H]²⁺: 1063.6.

### Preparation of Example 534

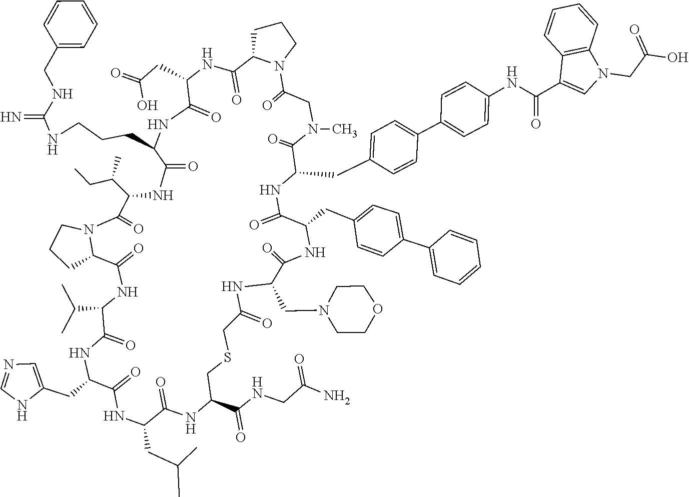 [SMILES: CC[C@H](C)[C@@H]1NC(=O)[C@@H](CCCNC(=N)NCc2ccccc2)NC(=O)[C@H](CC(=O)O)NC(=O)[C@@H]2CCCN2C(=O)CN(C)C(=O)[C@H](Cc2ccc(-c3ccc(NC(=O)c4cn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 534 was prepared on a 50 umol scale. It was purified by Preparative LC/MS. The yield of the product was 27.7 mg, and its estimated purity by LCMS analysis was 99.1%. Analysis condition 2: Retention time=1.58 min. ESI-MS(+) m/z [M+2H]²⁺: 1063.8.

### Preparation of Example 535

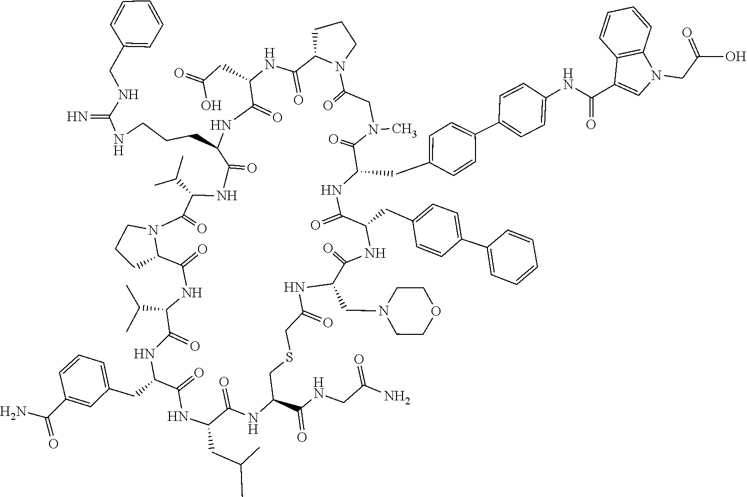 [SMILES: CC(C)C[C@@H]1NC(=O)[C@H](Cc2cccc(C(N)=O)c2)NC(=O)[C@H](C(C)C)NC(=O)[C@@H]2CCCN2C(=O)[C@H](C(C)C)NC(=O)[C@@H](CCCNC(=N)NCc2ccccc2)NC(=O)[C@H](CC(=O)O)NC(=O)[C@@H]2CCCN2C(=O)CN(C)C(=O)[C@H](Cc2ccc(-c3ccc(NC(=O)c4cn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 535 was prepared on a 50 umol scale. It was purified by Preparative LC/MS. The yield of the product was 6.1 mg, and its estimated purity by LCMS analysis was 100%. Analysis condition 1: Retention time=1.42 min. ESI-MS(+) m/z [M+2H]²⁺: 1083.3.

### Preparation of Example 536

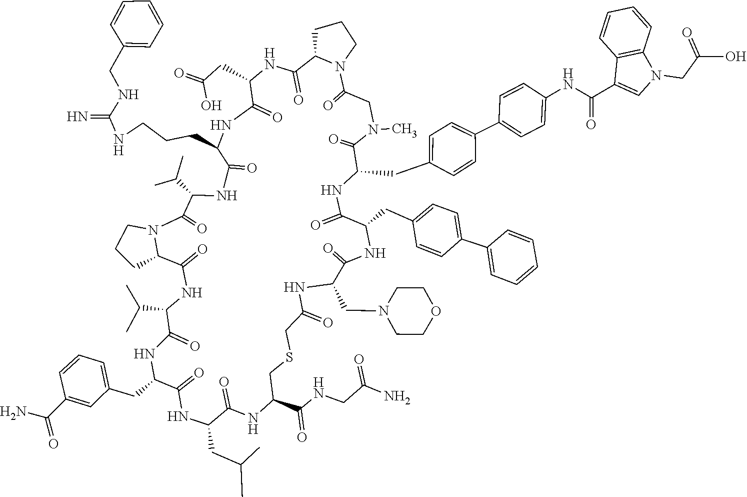 [SMILES: CC(C)C[C@@H]1NC(=O)[C@H](Cc2cccc(C(N)=O)c2)NC(=O)[C@H](C(C)C)NC(=O)[C@@H]2CCCN2C(=O)[C@H](C(C)C)NC(=O)[C@@H](CCCNC(=N)NCc2ccccc2)NC(=O)[C@H](CC(=O)O)NC(=O)[C@@H]2CCCN2C(=O)CN(C)C(=O)[C@H](Cc2ccc(-c3ccc(NC(=O)c4cn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 536 was prepared on a 50 umol scale. It was purified by Preparative LC/MS. The yield of the product was 6.9 mg, and its estimated purity by LCMS analysis was 100%. Analysis condition: Retention time=1.52 min. ESI-MS(+) m/z [M+2H]²⁺: 1083.4.

### Preparation of Example 537

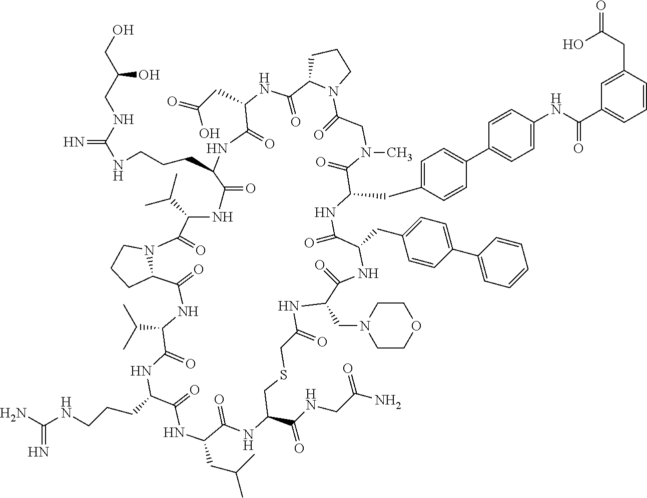 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NC[C@H](O)CO)NC(=O)[C@H](CC(=O)O)NC(=O)[C@@H]2CCCN2C(=O)CN(C)C(=O)[C@H](Cc2ccc(-c3ccc(NC(=O)c4cccc(CC(=O)O)c4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 537 was prepared on a 50 umol scale. It was purified by Preparative LC/MS. The yield of the product was 9.7 mg, and its estimated purity by LCMS analysis was 91.6%. Analysis condition 1: Retention time=1.29 min. ESI-MS(+) m/z [M+2H]²⁺: 1038.5.

### Preparation of Example 538

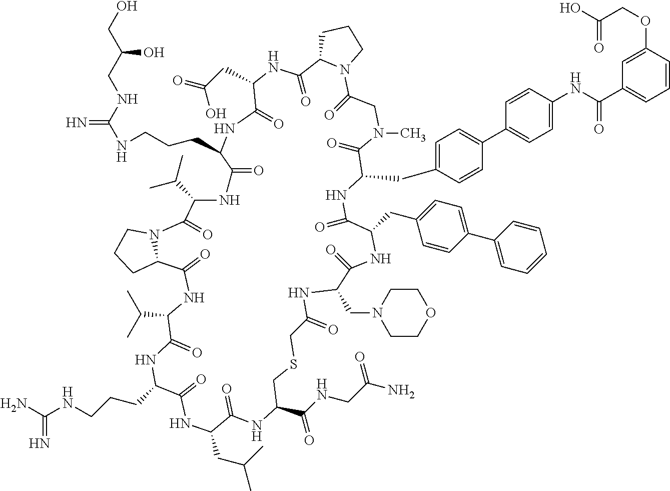 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NC[C@H](O)CO)NC(=O)[C@H](CC(=O)O)NC(=O)[C@@H]2CCCN2C(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 538 was prepared on a 50 umol scale. It was purified by Preparative LC/MS. The yield of the product was 14.4 mg, and its estimated purity by LCMS analysis was 87.7%. Analysis condition 1: Retention time=1.26 min. ESI-MS(+) m/z [M+2H]²⁺: 1046.7.

### Preparation of Example 539

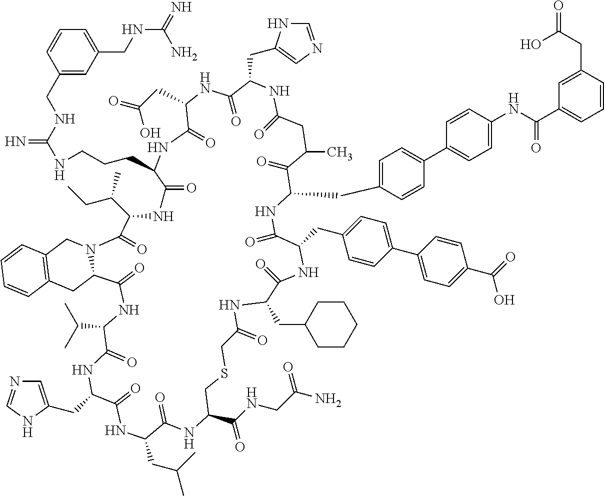 [SMILES: CC[C@H](C)[C@@H]1NC(=O)[C@@H](CCCNC(=N)NCc2cccc(CNC(=N)N)c2)NC(=O)[C@H](CC(=O)O)NC(=O)[C@H](Cc2cnc[nH]2)NC(=O)CC(C)C(=O)[C@H](Cc2ccc(-c3ccc(NC(=O)c4cccc(CC(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2Cc3ccccc3CN2C1=O]

Example 539 was prepared on a 50 umol scale. It was purified by Preparative LC/MS. The yield of the product was 9.7 mg, and its estimated purity by LCMS analysis was 87.6%. Analysis condition 2: Retention time=1.35 min. ESI-MS(+) m/z [M+2H]²⁺: 1151.3.

### Preparation of Example 540

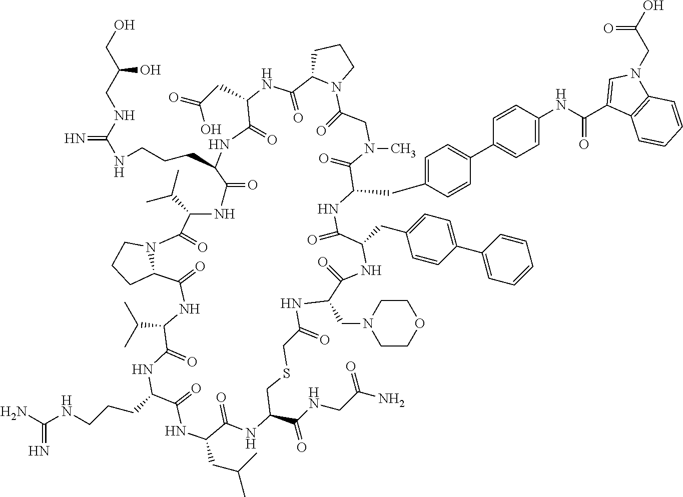 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NC[C@H](O)CO)NC(=O)[C@H](CC(=O)O)NC(=O)[C@@H]2CCCN2C(=O)CN(C)C(=O)[C@H](Cc2ccc(-c3ccc(NC(=O)c4cn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 540 was prepared on a 50 umol scale. It was purified by Preparative LC/MS. The yield of the product was 6.1 mg, and its estimated purity by LCMS analysis was 98.1%. Analysis condition 2: Retention time=1.42 min. ESI-MS(+) m/z [M+2H]²⁺: 1057.9.

### Preparation of Example 541

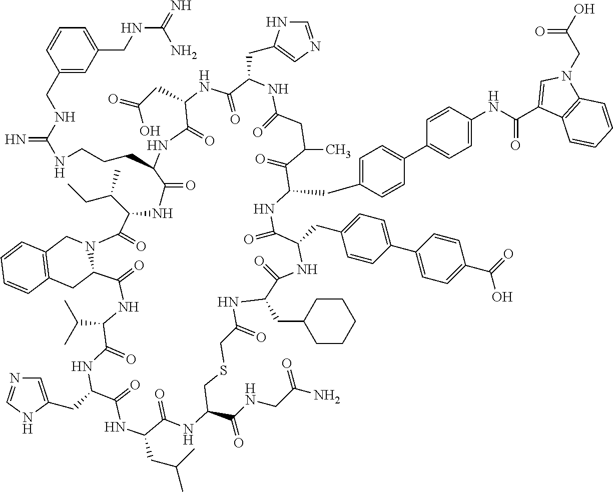 [SMILES: CC[C@H](C)[C@@H]1NC(=O)[C@@H](CCCNC(=N)NCc2cccc(CNC(=N)N)c2)NC(=O)[C@H](CC(=O)O)NC(=O)[C@H](Cc2cnc[nH]2)NC(=O)CC(C)C(=O)[C@H](Cc2ccc(-c3ccc(NC(=O)c4cn(CC(=O)O)c5ccccc45)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2Cc3ccccc3CN2C1=O]

Example 541 was prepared on a 50 umol scale. It was purified by Preparative LC/MS. The yield of the product was 6.1 mg, and its estimated purity by LCMS analysis was 88.4%. Analysis condition 2: Retention time=1.39 min. ESI-MS(+) m/z [M+2H]²⁺: 1170.8.

### Preparation of Example 542

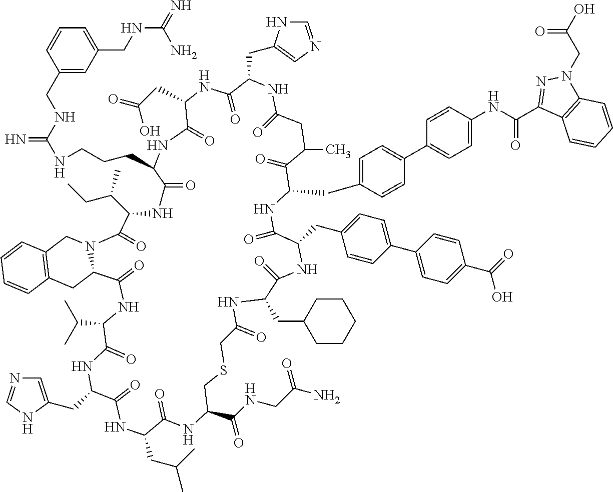 [SMILES: CC[C@H](C)[C@@H]1NC(=O)[C@@H](CCCNC(=N)NCc2cccc(CNC(=N)N)c2)NC(=O)[C@H](CC(=O)O)NC(=O)[C@H](Cc2cnc[nH]2)NC(=O)CC(C)C(=O)[C@H](Cc2ccc(-c3ccc(NC(=O)c4nn(CC(=O)O)c5ccccc45)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2Cc3ccccc3CN2C1=O]

Example 542 was prepared on a 100 umol scale. It was purified by Preparative LC/MS. The yield of the product was 30.9 mg, and its estimated purity by LCMS analysis was 91%. Analysis condition 2: Retention time=1.4 min. ESI-MS(+) m/z [M+2H]²⁺: 1171.3.

### Preparation of Example 543

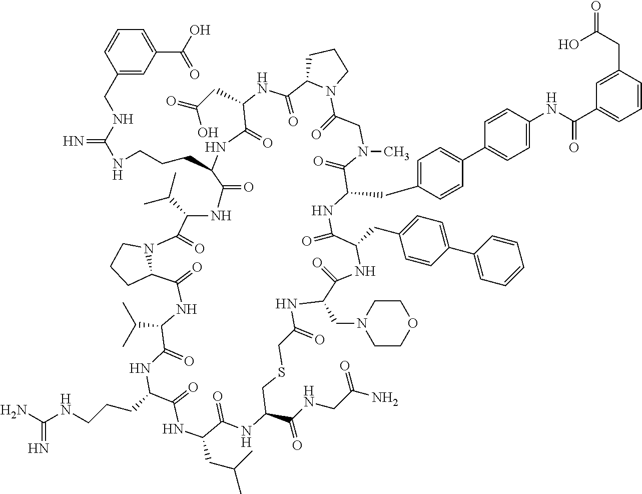 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2cccc(C(=O)O)c2)NC(=O)[C@H](CC(=O)O)NC(=O)[C@@H]2CCCN2C(=O)CN(C)C(=O)[C@H](Cc2ccc(-c3ccc(NC(=O)c4cccc(CC(=O)O)c4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 543 was prepared on a 50 Preparative LC/MS. The yield of the product was 1.8 mg, and its estimated purity by LCMS analysis was 93.3%. Analysis condition 2: Retention time=1.42 min. ESI-MS(+) m/z [M+2H]²⁺: 1068.1.

### Preparation of Example 544

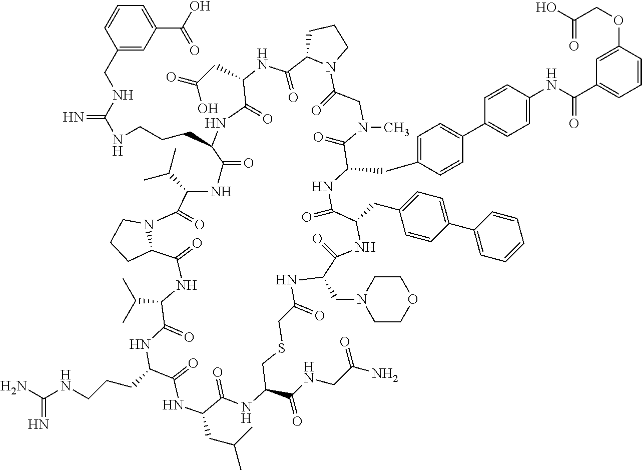 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2cccc(C(=O)O)c2)NC(=O)[C@H](CC(=O)O)NC(=O)[C@@H]2CCCN2C(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 544 was prepared on a 50 umol scale. It was purified by Preparative LC/MS. The yield of the product was 1.4 mg, and its estimated purity by LCMS analysis was 91.3%. Analysis condition 2: Retention time=1.43 min. ESI-MS(+) m/z [M+2H]²⁺: 1076.2.

### Preparation of Example 545

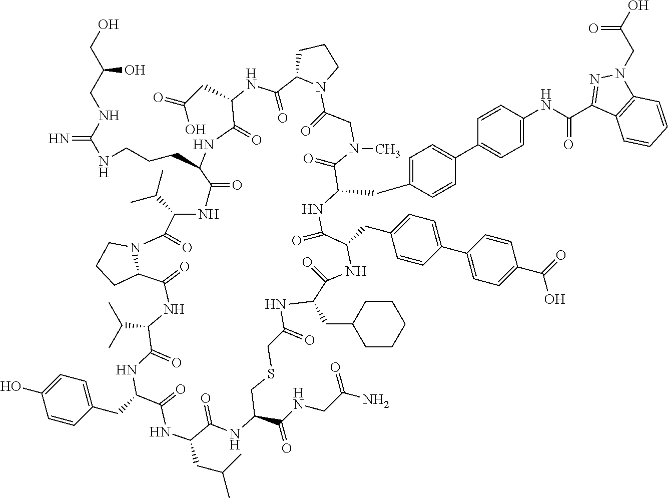 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)NC[C@H](O)CO)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 545 was prepared on a 50 Preparative LC/MS. The yield of the product was 4 mg, and its estimated purity by LCMS analysis was 90.5%. Analysis condition 1: Retention time=1.19 min. ESI-MS(+) m/z [M+2H]²⁺: 1082.

### Preparation of Example 546

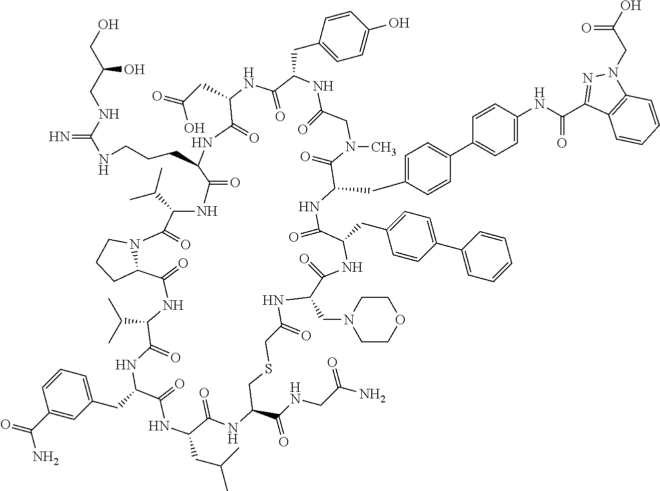 [SMILES: CC(C)C[C@@H]1NC(=O)[C@H](Cc2cccc(C(N)=O)c2)NC(=O)[C@H](C(C)C)NC(=O)[C@@H]2CCCN2C(=O)[C@H](C(C)C)NC(=O)[C@@H](CCCNC(=N)NC[C@H](O)CO)NC(=O)[C@H](CC(=O)O)NC(=O)[C@H](Cc2ccc(O)cc2)NC(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 546 was prepared on a 50 umol scale. It was purified by Preparative LC/MS. The yield of the product was 7.8 mg, and its estimated purity by LCMS analysis was 95.5%. Analysis condition 2: Retention time=1.41 min. ESI-MS(+) m/z [M+2H]²⁺: 1108.6.

### Preparation of Example 547

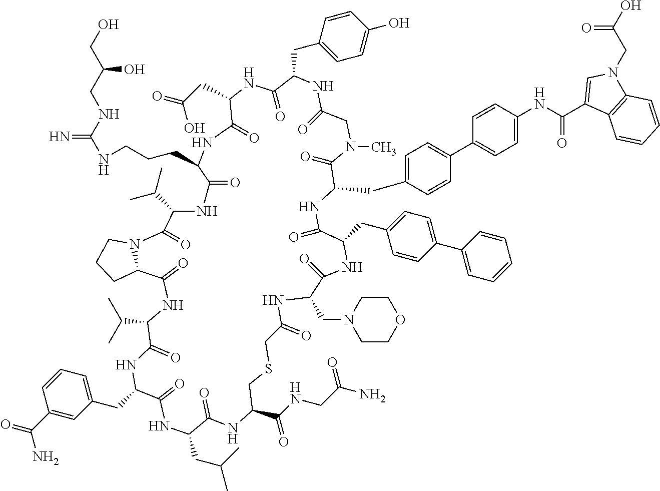 [SMILES: CC(C)C[C@@H]1NC(=O)[C@H](Cc2cccc(C(N)=O)c2)NC(=O)[C@H](C(C)C)NC(=O)[C@@H]2CCCN2C(=O)[C@H](C(C)C)NC(=O)[C@@H](CCCNC(=N)NC[C@H](O)CO)NC(=O)[C@H](CC(=O)O)NC(=O)[C@H](Cc2ccc(O)cc2)NC(=O)CN(C)C(=O)[C@H](Cc2ccc(-c3ccc(NC(=O)c4cn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 547 was prepared on a 50 Preparative LC/MS. The yield of the product was 3.8 mg, and its estimated purity by LCMS analysis was 93.1%. Analysis condition 2: Retention time=1.38 min. ESI-MS(+) m/z [M+2H]²⁺: 1108.

### Preparation of Example 548

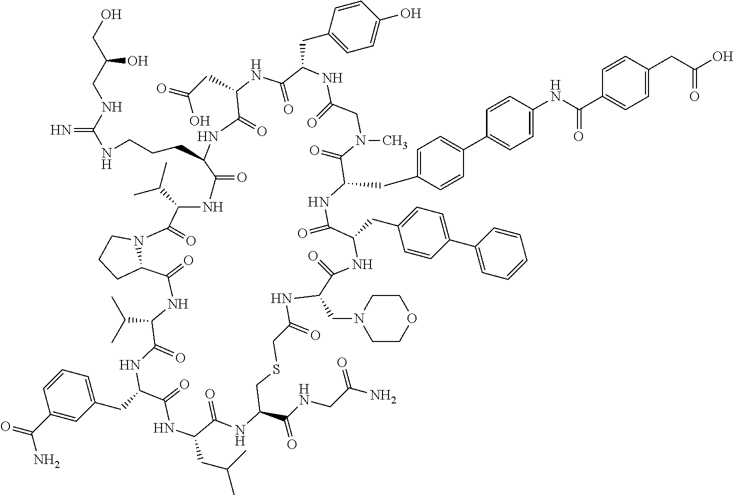 [SMILES: CC(C)C[C@@H]1NC(=O)[C@H](Cc2cccc(C(N)=O)c2)NC(=O)[C@H](C(C)C)NC(=O)[C@@H]2CCCN2C(=O)[C@H](C(C)C)NC(=O)[C@@H](CCCNC(=N)NC[C@H](O)CO)NC(=O)[C@H](CC(=O)O)NC(=O)[C@H](Cc2ccc(O)cc2)NC(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 548 was prepared on a 50 umol scale. It was purified by Preparative LC/MS. The yield of the product was 5.1 mg, and its estimated purity by LCMS analysis was 97.1%. Analysis condition 1: Retention time=1.34 min. ESI-MS(+) m/z [M+2H]²⁺: 1088.4.

### Preparation of Example 549

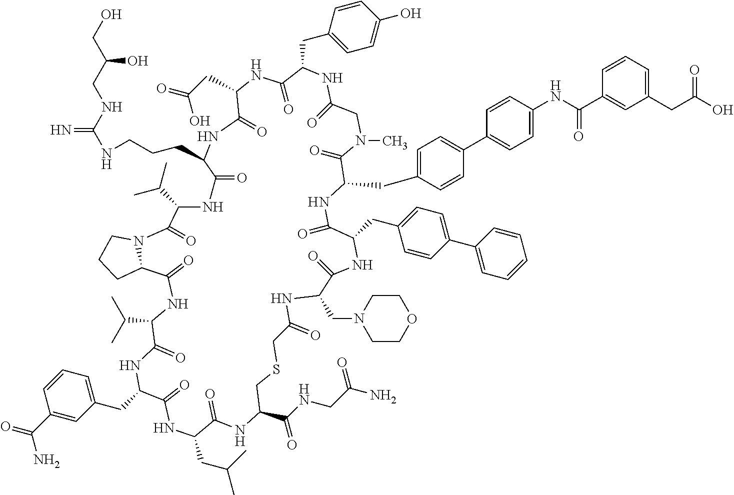 [SMILES: CC(C)C[C@@H]1NC(=O)[C@H](Cc2cccc(C(N)=O)c2)NC(=O)[C@H](C(C)C)NC(=O)[C@@H]2CCCN2C(=O)[C@H](C(C)C)NC(=O)[C@@H](CCCNC(=N)NC[C@H](O)CO)NC(=O)[C@H](CC(=O)O)NC(=O)[C@H](Cc2ccc(O)cc2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 549 was prepared on a 50 Preparative LC/MS. The yield of the product was 4.6 mg, and its estimated purity by LCMS analysis was 92%. Analysis condition 2: Retention time=1.46 min. ESI-MS(+) m/z [M+2H]²⁺: 1088.6.

### Preparation of Example 550

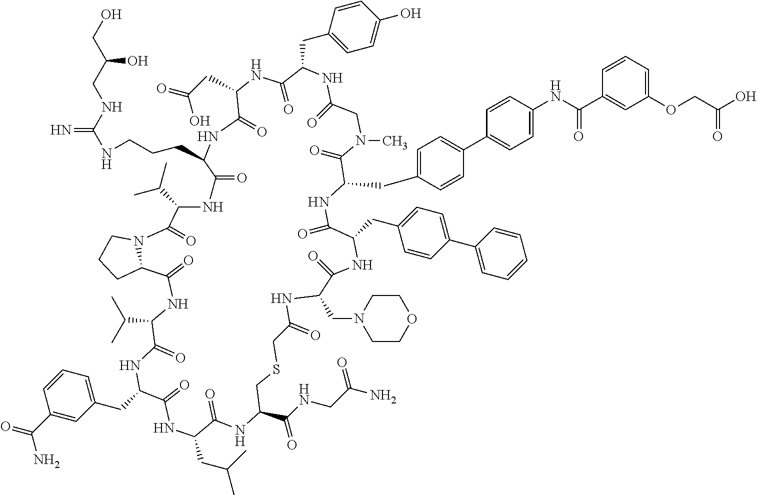 [SMILES: CC(C)C[C@@H]1NC(=O)[C@H](Cc2cccc(C(N)=O)c2)NC(=O)[C@H](C(C)C)NC(=O)[C@@H]2CCCN2C(=O)[C@H](C(C)C)NC(=O)[C@@H](CCCNC(=N)NC[C@H](O)CO)NC(=O)[C@H](CC(=O)O)NC(=O)[C@H](Cc2ccc(O)cc2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 550 was prepared on a 50 umol scale. It was purified by Preparative LC/MS. The yield of the product was 4.7 mg, and its estimated purity by LCMS analysis was 89.6%. Analysis condition 2: Retention time=1.46 min. ESI-MS(+) m/z [M+2H]²⁺: 1096.2.

### Preparation of Example 551

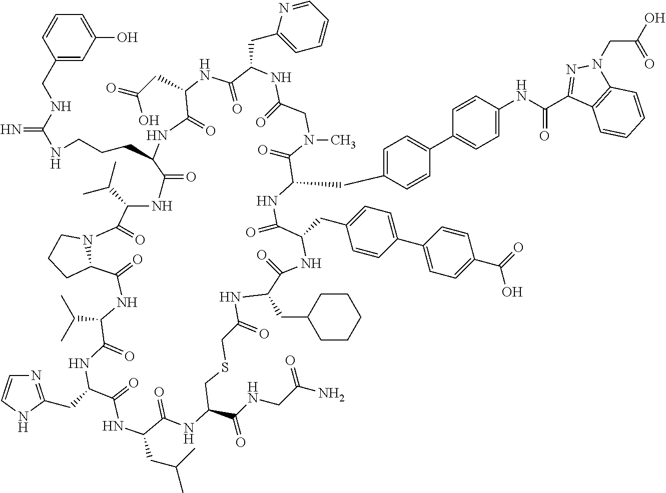 [SMILES: CC(C)C[C@@H]1NC(=O)[C@H](Cc2ncc[nH]2)NC(=O)[C@H](C(C)C)NC(=O)[C@@H]2CCCN2C(=O)[C@H](C(C)C)NC(=O)[C@@H](CCCNC(=N)NCc2cccc(O)c2)NC(=O)[C@H](CC(=O)O)NC(=O)[C@H](Cc2ccccn2)NC(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 551 was prepared on a 50 Preparative LC/MS. The yield of the product was 12.5 mg, and its estimated purity by LCMS analysis was 90.6%. Analysis condition 1: Retention time=1.34 min. ESI-MS(+) m/z [M+2H]²⁺: 1111.

### Preparation of Example 552

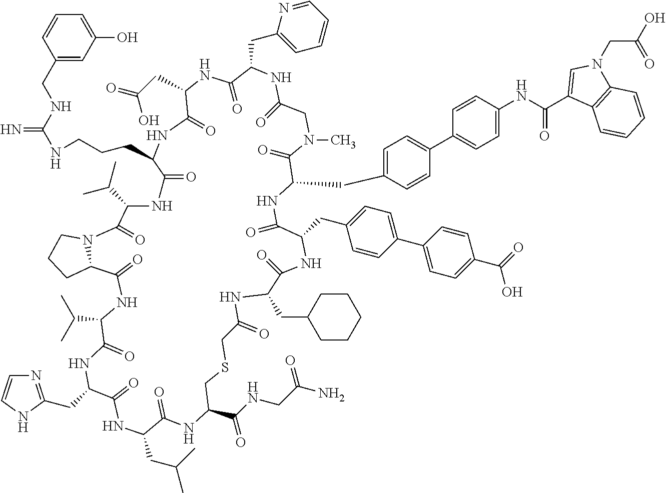 [SMILES: CC(C)C[C@@H]1NC(=O)[C@H](Cc2ncc[nH]2)NC(=O)[C@H](C(C)C)NC(=O)[C@@H]2CCCN2C(=O)[C@H](C(C)C)NC(=O)[C@@H](CCCNC(=N)NCc2cccc(O)c2)NC(=O)[C@H](CC(=O)O)NC(=O)[C@H](Cc2ccccn2)NC(=O)CN(C)C(=O)[C@H](Cc2ccc(-c3ccc(NC(=O)c4cn(CC(=O)O)c5ccccc45)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 552 was prepared on a 50 umol scale. It was purified by Preparative LC/MS. The yield of the product was 2.7 mg, and its estimated purity by LCMS analysis was 100%. Analysis condition 2: Retention time=1.35 min. ESI-MS(+) m/z [M+2H]²⁺: 1110.7.

### Preparation of Example 553

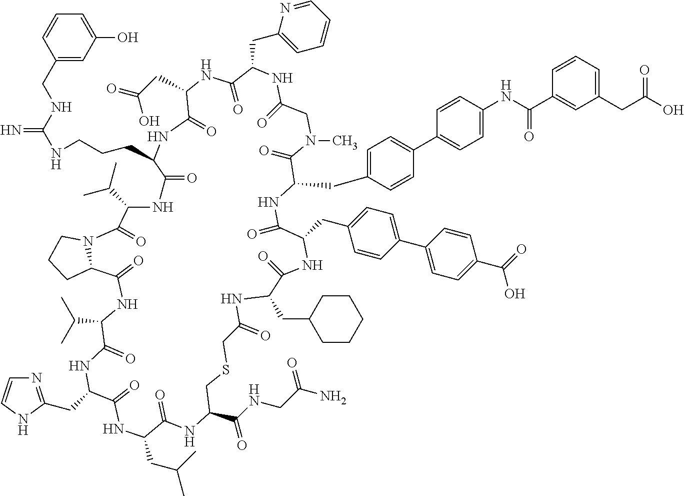 [SMILES: CC(C)C[C@@H]1NC(=O)[C@H](Cc2ncc[nH]2)NC(=O)[C@H](C(C)C)NC(=O)[C@@H]2CCCN2C(=O)[C@H](C(C)C)NC(=O)[C@@H](CCCNC(=N)NCc2cccc(O)c2)NC(=O)[C@H](CC(=O)O)NC(=O)[C@H](Cc2ccccn2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 553 was prepared on a 50 Preparative LC/MS. The yield of the product was 7 mg, and its estimated purity by LCMS analysis was 100%. Analysis condition 2: Retention time=1.29 min. ESI-MS(+) m/z [M+2H]²⁺: 1090.9.

### Preparation of Example 554

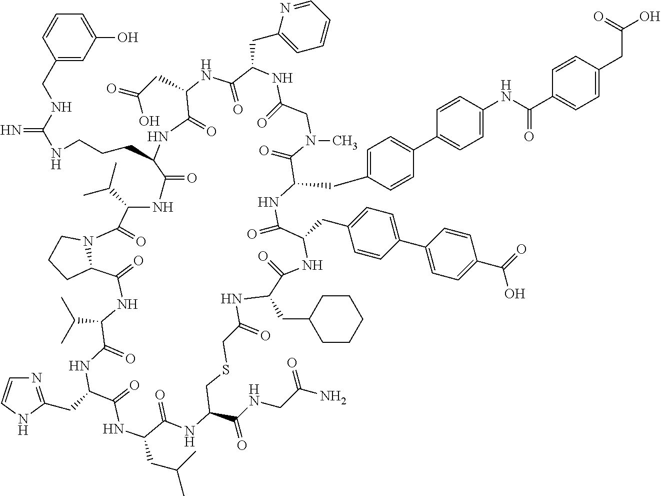 [SMILES: CC(C)C[C@@H]1NC(=O)[C@H](Cc2ncc[nH]2)NC(=O)[C@H](C(C)C)NC(=O)[C@@H]2CCCN2C(=O)[C@H](C(C)C)NC(=O)[C@@H](CCCNC(=N)NCc2cccc(O)c2)NC(=O)[C@H](CC(=O)O)NC(=O)[C@H](Cc2ccccn2)NC(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 554 was prepared on a 50 umol scale. It was purified by Preparative LC/MS. The yield of the product was 8.1 mg, and its estimated purity by LCMS analysis was 91.9%. Analysis condition 1: Retention time=1.22 min. ESI-MS(+) m/z [M+2H]²⁺: 1090.7.

### Preparation of Example 555

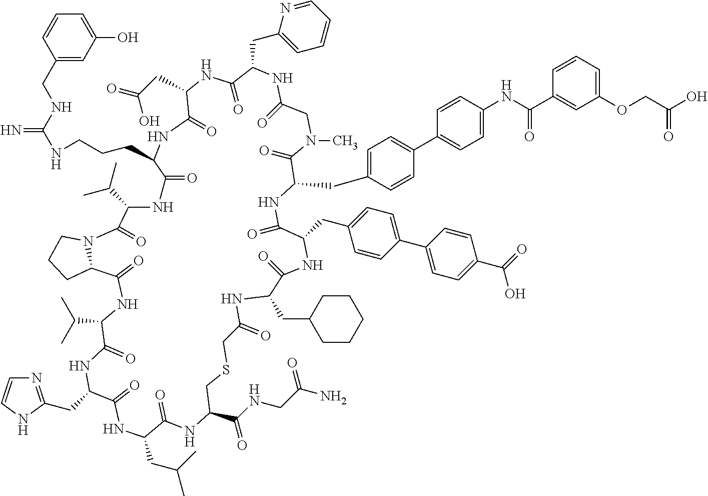 [SMILES: CC(C)C[C@@H]1NC(=O)[C@H](Cc2ncc[nH]2)NC(=O)[C@H](C(C)C)NC(=O)[C@@H]2CCCN2C(=O)[C@H](C(C)C)NC(=O)[C@@H](CCCNC(=N)NCc2cccc(O)c2)NC(=O)[C@H](CC(=O)O)NC(=O)[C@H](Cc2ccccn2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 555 was prepared on a 50 Preparative LC/MS. The yield of the product was 3.2 mg, and its estimated purity by LCMS analysis was 94.9%. Analysis condition 1: Retention time=1.2 min. ESI-MS(+) m/z [M+2H]²⁺: 1099.4.

### Preparation of Example 556

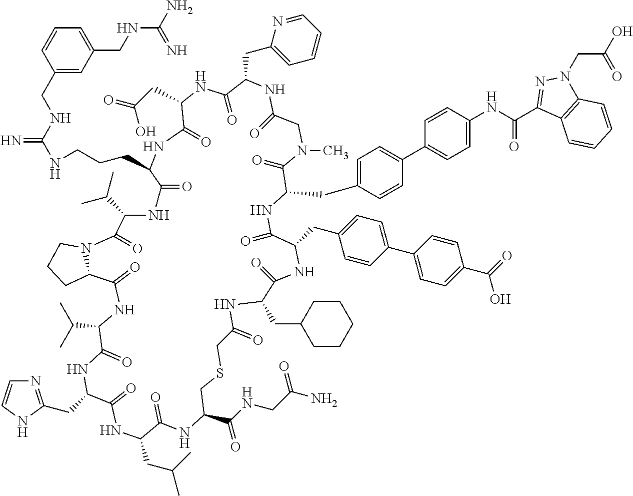 [SMILES: CC(C)C[C@@H]1NC(=O)[C@H](Cc2ncc[nH]2)NC(=O)[C@H](C(C)C)NC(=O)[C@@H]2CCCN2C(=O)[C@H](C(C)C)NC(=O)[C@@H](CCCNC(=N)NCc2cccc(CNC(=N)N)c2)NC(=O)[C@H](CC(=O)O)NC(=O)[C@H](Cc2ccccn2)NC(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 556 was prepared on a 50 umol scale. It was purified by Preparative LC/MS. The yield of the product was 12.8 mg, and its estimated purity by LCMS analysis was 90.6%. Analysis condition 1: Retention time=1.31 min. ESI-MS(+) m/z [M+2H]²⁺: 1138.5.

### Preparation of Example 557

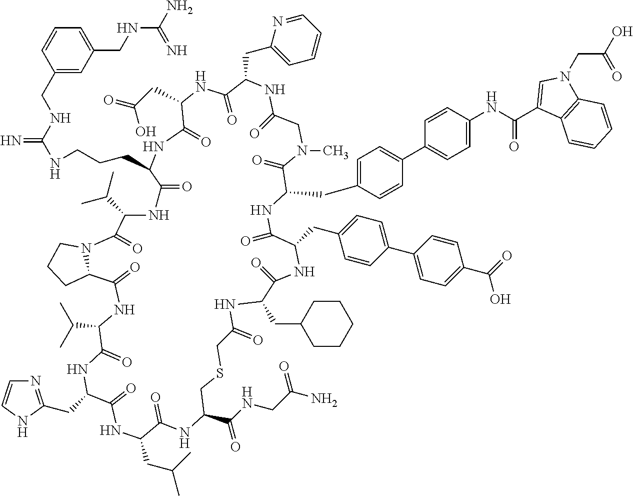 [SMILES: CC(C)C[C@@H]1NC(=O)[C@H](Cc2ncc[nH]2)NC(=O)[C@H](C(C)C)NC(=O)[C@@H]2CCCN2C(=O)[C@H](C(C)C)NC(=O)[C@@H](CCCNC(=N)NCc2cccc(CNC(=N)N)c2)NC(=O)[C@H](CC(=O)O)NC(=O)[C@H](Cc2ccccn2)NC(=O)CN(C)C(=O)[C@H](Cc2ccc(-c3ccc(NC(=O)c4cn(CC(=O)O)c5ccccc45)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 557 was prepared on a 50 umol scale. It was purified by Preparative LC/MS. The yield of the product was 2.6 mg, and its estimated purity by LCMS analysis was 92.4%. Analysis condition 1: Retention time=1.22 min. ESI-MS(+) m/z [M+2H]²⁺: 1138.

### Preparation of Example 558

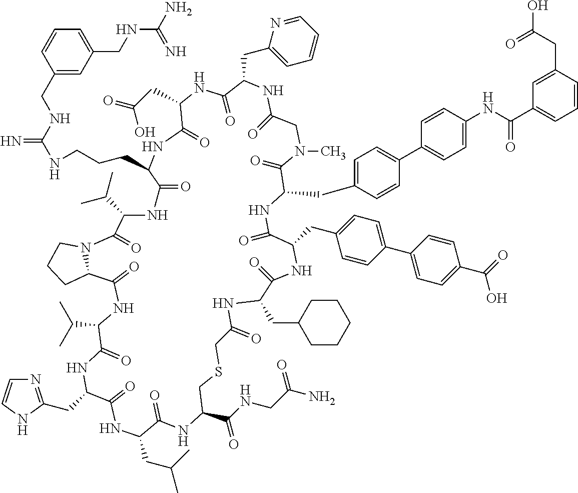 [SMILES: CC(C)C[C@@H]1NC(=O)[C@H](Cc2ncc[nH]2)NC(=O)[C@H](C(C)C)NC(=O)[C@@H]2CCCN2C(=O)[C@H](C(C)C)NC(=O)[C@@H](CCCNC(=N)NCc2cccc(CNC(=N)N)c2)NC(=O)[C@H](CC(=O)O)NC(=O)[C@H](Cc2ccccn2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 558 was prepared on a 50 umol scale. It was purified by Preparative LC/MS. The yield of the product was 5.9 mg, and its estimated purity by LCMS analysis was 90.1%. Analysis condition 2: Retention time=1.41 min. ESI-MS(+) m/z [M+2H]²⁺: 1118.5.

### Preparation of Example 559

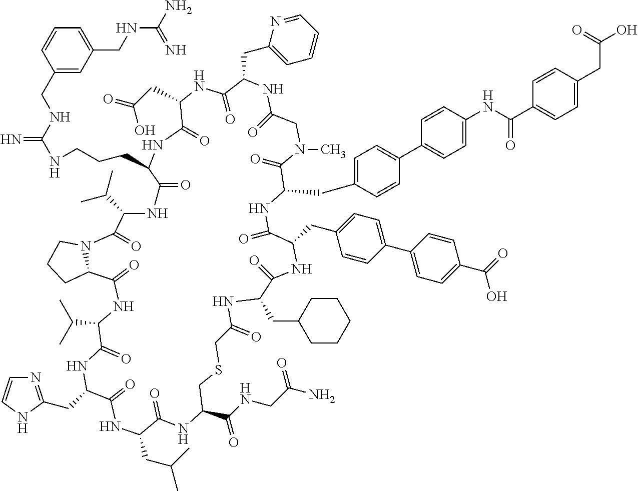 [SMILES: CC(C)C[C@@H]1NC(=O)[C@H](Cc2ncc[nH]2)NC(=O)[C@H](C(C)C)NC(=O)[C@@H]2CCCN2C(=O)[C@H](C(C)C)NC(=O)[C@@H](CCCNC(=N)NCc2cccc(CNC(=N)N)c2)NC(=O)[C@H](CC(=O)O)NC(=O)[C@H](Cc2ccccn2)NC(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 559 was prepared on a 50 Preparative LC/MS. The yield of the product was 4.1 mg, and its estimated purity by LCMS analysis was 98.2%. Analysis condition 1: Retention time=1.14 min. ESI-MS(+) m/z [M+2H]²⁺: 1118.5.

### Preparation of Example 560

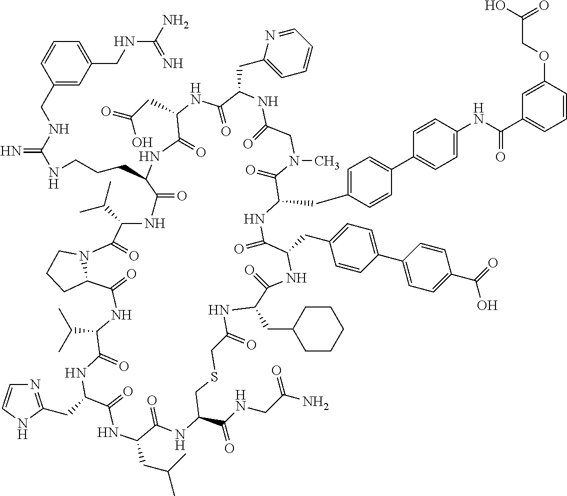 [SMILES: CC(C)C[C@@H]1NC(=O)[C@H](Cc2ncc[nH]2)NC(=O)[C@H](C(C)C)NC(=O)[C@@H]2CCCN2C(=O)[C@H](C(C)C)NC(=O)[C@@H](CCCNC(=N)NCc2cccc(CNC(=N)N)c2)NC(=O)[C@H](CC(=O)O)NC(=O)[C@H](Cc2ccccn2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 560 was prepared on a 50 umol scale. It was purified by Preparative LC/MS. The yield of the product was 4.6 mg, and its estimated purity by LCMS analysis was 92%. Analysis condition 2: Retention time=1.31 min. ESI-MS(+) m/z [M+2H]²⁺: 1126.5.

### Preparation of Example 561

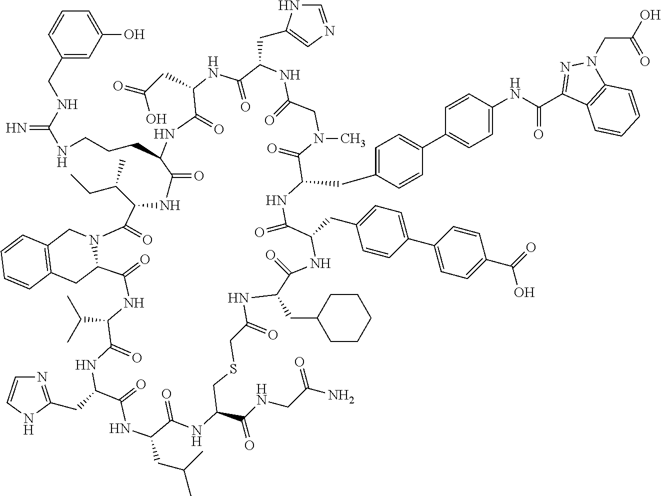 [SMILES: CC[C@H](C)[C@@H]1NC(=O)[C@@H](CCCNC(=N)NCc2cccc(O)c2)NC(=O)[C@H](CC(=O)O)NC(=O)[C@H](Cc2cnc[nH]2)NC(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2ncc[nH]2)NC(=O)[C@H](C(C)C)NC(=O)[C@@H]2Cc3ccccc3CN2C1=O]

Example 561 was prepared on a 50 umol scale. It was purified by Preparative LC/MS. The yield of the product was 11 mg, and its estimated purity by LCMS analysis was 93.7%. Analysis condition 1: Retention time=1.38 min. ESI-MS(+) m/z [M+2H]²⁺: 1143.4.

### Preparation of Example 562

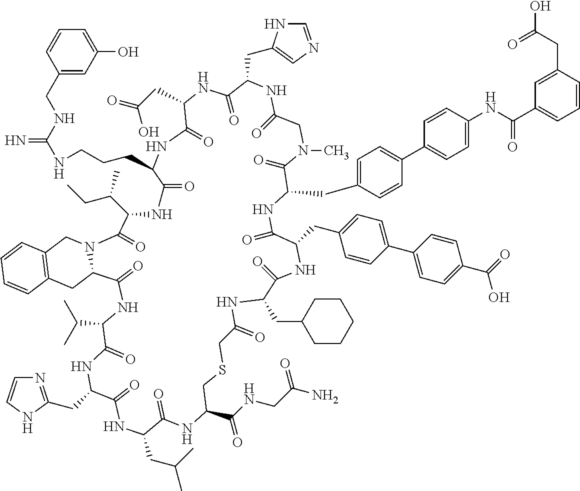 [SMILES: CC[C@H](C)[C@@H]1NC(=O)[C@@H](CCCNC(=N)NCc2cccc(O)c2)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2ncc[nH]2)NC(=O)[C@H](C(C)C)NC(=O)[C@@H]2Cc3ccccc3CN2C1=O]

Example 562 was prepared on a 50 umol scale. It was purified by Preparative LC/MS. The yield of the product was 21.4 mg, and its estimated purity by LCMS analysis was 93%. Analysis condition 1: Retention time=1.35 min. ESI-MS(+) m/z [M+2H]²⁺: 1123.5.

### Preparation of Example 563

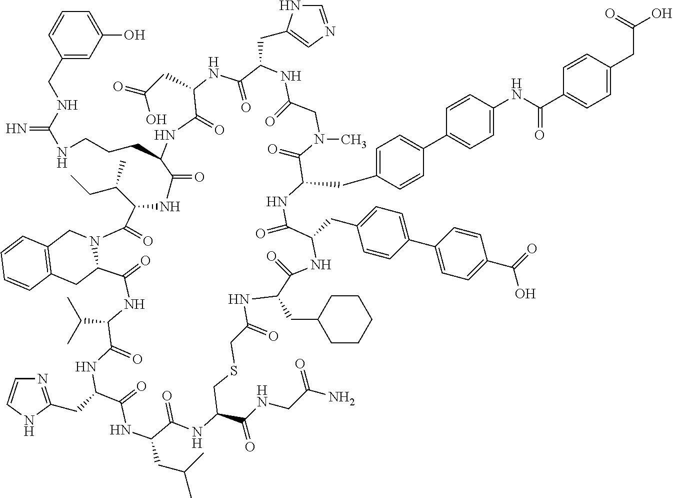 [SMILES: CC[C@H](C)[C@@H]1NC(=O)[C@@H](CCCNC(=N)NCc2cccc(O)c2)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2ncc[nH]2)NC(=O)[C@H](C(C)C)NC(=O)[C@@H]2Cc3ccccc3CN2C1=O]

Example 563 was prepared on a 50 Preparative LC/MS. The yield of the product was 9 mg, and its estimated purity by LCMS analysis was 95.1%. Analysis condition 1: Retention time=1.28 min. ESI-MS(+) m/z [M+2H]²⁺: 1123.4.

### Preparation of Example 564

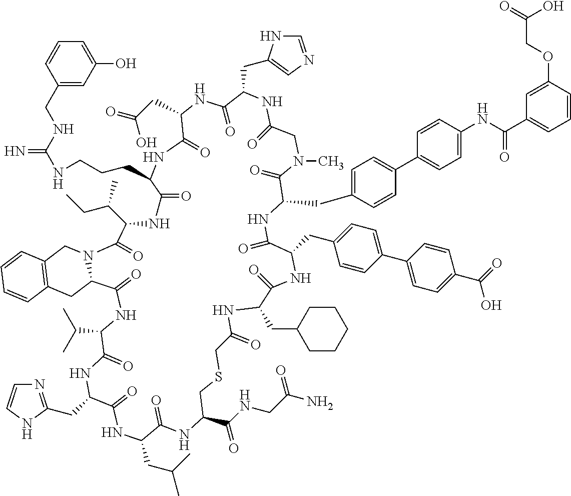 [SMILES: CC[C@H](C)[C@@H]1NC(=O)[C@@H](CCCNC(=N)NCc2cccc(O)c2)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2ncc[nH]2)NC(=O)[C@H](C(C)C)NC(=O)[C@@H]2Cc3ccccc3CN2C1=O]

Example 564 was prepared on a 50 umol scale. It was purified by Preparative LC/MS. The yield of the product was 19.1 mg, and its estimated purity by LCMS analysis was 90.9%. Analysis condition 1: Retention time=1.36 min. ESI-MS(+) m/z [M+2H]²⁺: 1131.5.

### Preparation of Example 565

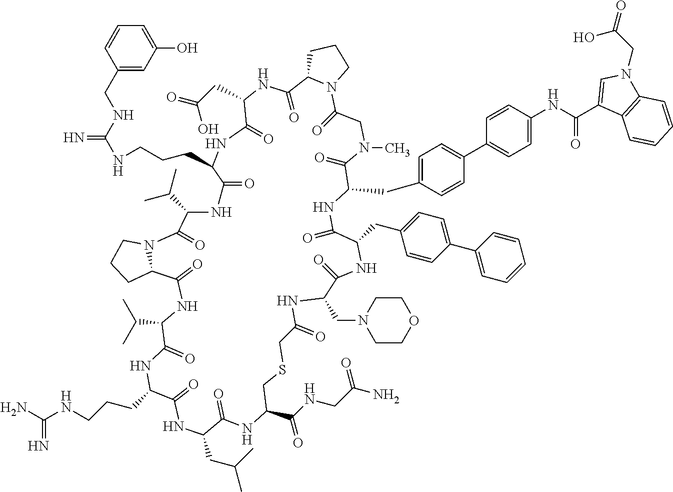 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2cccc(O)c2)NC(=O)[C@H](CC(=O)O)NC(=O)[C@@H]2CCCN2C(=O)CN(C)C(=O)[C@H](Cc2ccc(-c3ccc(NC(=O)c4cn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 565 was prepared on a 50 umol scale. It was purified by Preparative LC/MS. The yield of the product was 2.6 mg, and its estimated purity by LCMS analysis was 90.9%. Analysis condition 1: Retention time=1.43 min. ESI-MS(+) m/z [M+3H]³⁺: 716.2.

### Preparation of Example 566

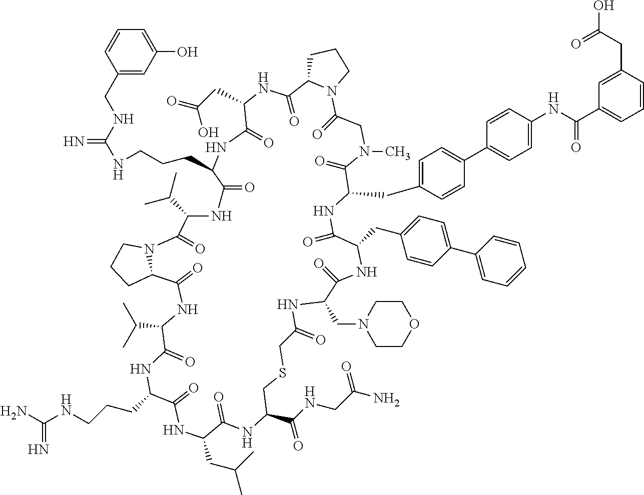 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2cccc(O)c2)NC(=O)[C@H](CC(=O)O)NC(=O)[C@@H]2CCCN2C(=O)CN(C)C(=O)[C@H](Cc2ccc(-c3ccc(NC(=O)c4cccc(CC(=O)O)c4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 566 was prepared on a 50 umol scale. It was purified by Preparative LC/MS. The yield of the product was 15.3 mg, and its estimated purity by LCMS analysis was 90.2%. Analysis condition 1: Retention time=1.45 min. ESI-MS(+) m/z [M+2H]²⁺: 1054.5.

### Preparation of Example 567

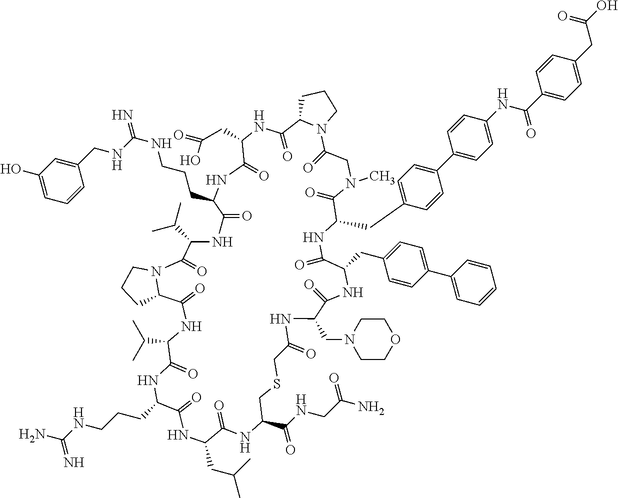 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2cccc(O)c2)NC(=O)[C@H](CC(=O)O)NC(=O)[C@@H]2CCCN2C(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 567 was prepared on a 50 umol scale. It was purified by Preparative LC/MS. The yield of the product was 4.5 mg, and its estimated purity by LCMS analysis was 93.6%. Analysis condition 1: Retention time=1.34 min. ESI-MS(+) m/z [M+2H]²⁺: 1054.4.

### Preparation of Example 568

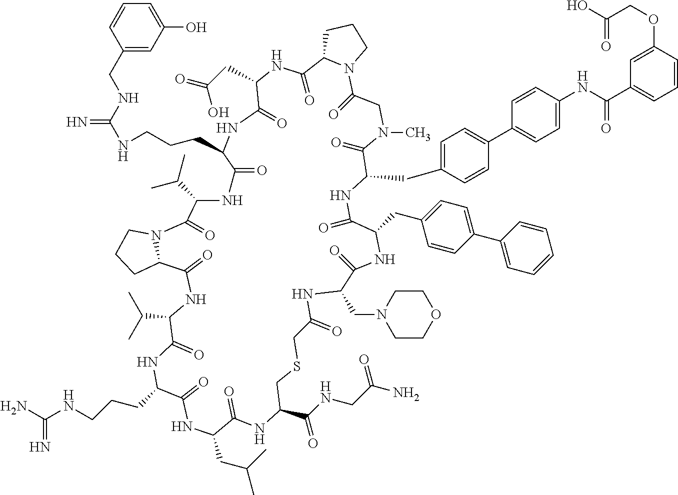 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2cccc(O)c2)NC(=O)[C@H](CC(=O)O)NC(=O)[C@@H]2CCCN2C(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 568 was prepared on a 50 umol scale. It was purified by Preparative LC/MS. The yield of the product was 15 mg, and its estimated purity by LCMS analysis was 100%. Analysis condition 1: Retention time=1.4 min. ESI-MS(+) m/z [M+2H]²⁺: 1062.6.

### Preparation of Example 569

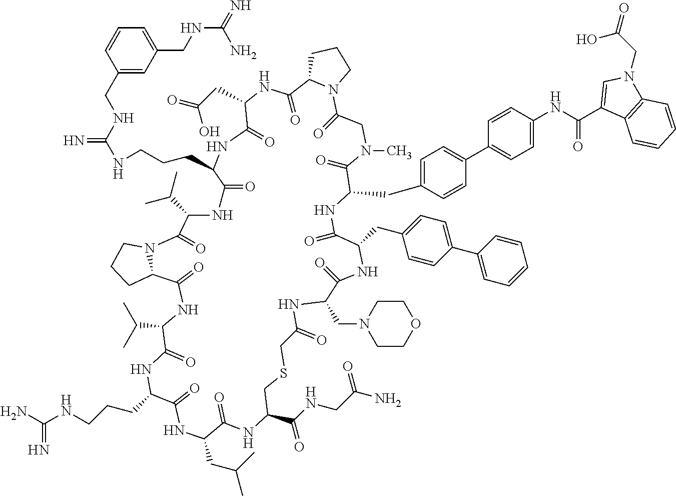 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2cccc(CNC(=N)N)c2)NC(=O)[C@H](CC(=O)O)NC(=O)[C@@H]2CCCN2C(=O)CN(C)C(=O)[C@H](Cc2ccc(-c3ccc(NC(=O)c4cn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 569 was prepared on a 50 umol scale. It was purified by Preparative LC/MS. The yield of the product was 2.5 mg, and its estimated purity by LCMS analysis was 94.4%. Analysis condition 2: Retention time=1.36 min. ESI-MS(+) m/z [M+2H]²⁺: 1101.4.

### Preparation of Example 570

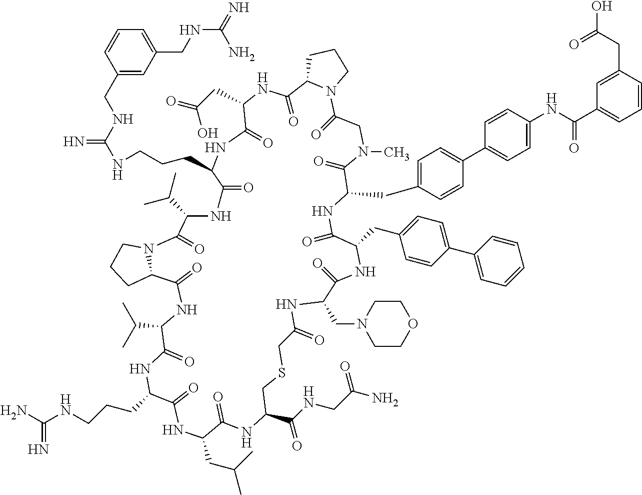 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2cccc(CNC(=N)N)c2)NC(=O)[C@H](CC(=O)O)NC(=O)[C@@H]2CCCN2C(=O)CN(C)C(=O)[C@H](Cc2ccc(-c3ccc(NC(=O)c4cccc(CC(=O)O)c4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 570 was prepared on a 50 umol scale. It was purified by Preparative LC/MS. The yield of the product was 2 mg, and its estimated purity by LCMS analysis was 100%. Analysis condition 2: Retention time=1.37 min. ESI-MS(+) m/z [M+2H]^{2+: 1082.2}.

### Preparation of Example 571

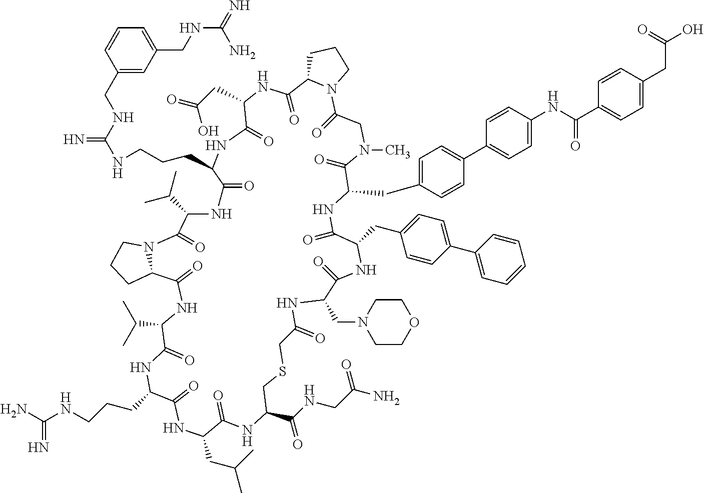 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2cccc(CNC(=N)N)c2)NC(=O)[C@H](CC(=O)O)NC(=O)[C@@H]2CCCN2C(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 571 was prepared on a 50 Preparative LC/MS. The yield of the product was 1.7 mg, and its estimated purity by LCMS analysis was 97.2%. Analysis condition 2: Retention time=1.32 min. ESI-MS(+) m/z [M+2H]²⁺: 1082.

### Preparation of Example 572

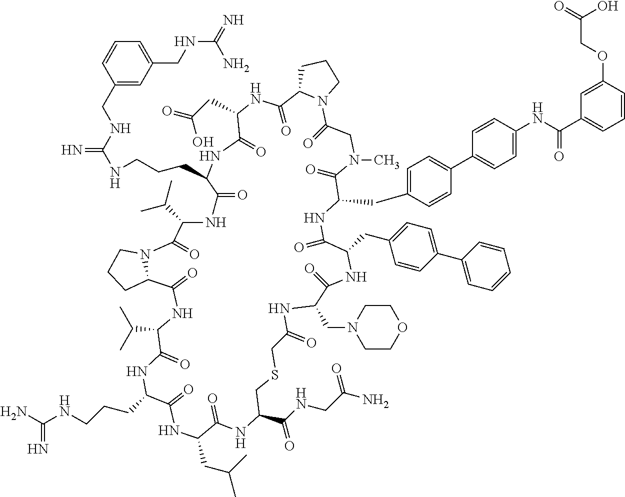 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2cccc(CNC(=N)N)c2)NC(=O)[C@H](CC(=O)O)NC(=O)[C@@H]2CCCN2C(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 572 was prepared on a 50 umol scale. It was purified by Preparative LC/MS. The yield of the product was 6.4 mg, and its estimated purity by LCMS analysis was 100%. Analysis condition 1: Retention time=1.34 min. ESI-MS(+) m/z [M+2H]²⁺: 1090.

### Preparation of Example 573

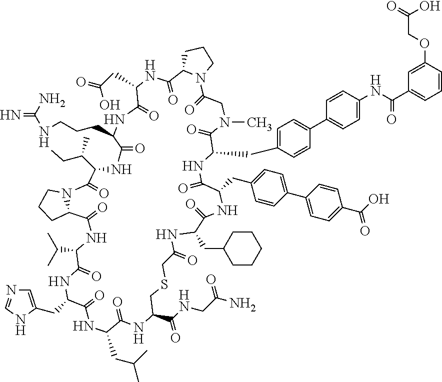 [SMILES: CC[C@H](C)[C@@H]1NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 573 was prepared on a 50 Preparative LC/MS. The yield of the product was 4.7 mg, and its estimated purity by LCMS analysis was 96.3%. Analysis condition 1: Retention time=1.16 min. ESI-MS(+) m/z [M+2H]²⁺: 1027.2.

### Preparation of Example 574

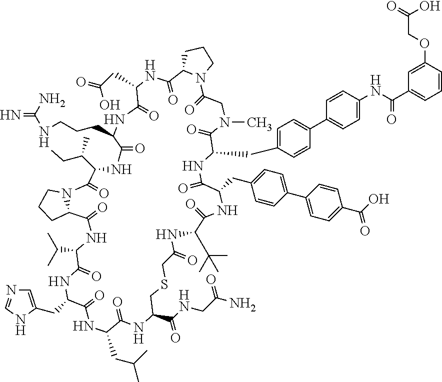 [SMILES: CC[C@H](C)[C@@H]1NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 574 was prepared on a 50 umol scale. It was purified by Preparative LC/MS. The yield of the product was 9.6 mg, and its estimated purity by LCMS analysis was 92%. Analysis condition 1: Retention time=1.1 min. ESI-MS(+) m/z [M+2H]²⁺: 1007.6.

### Preparation of Example 575

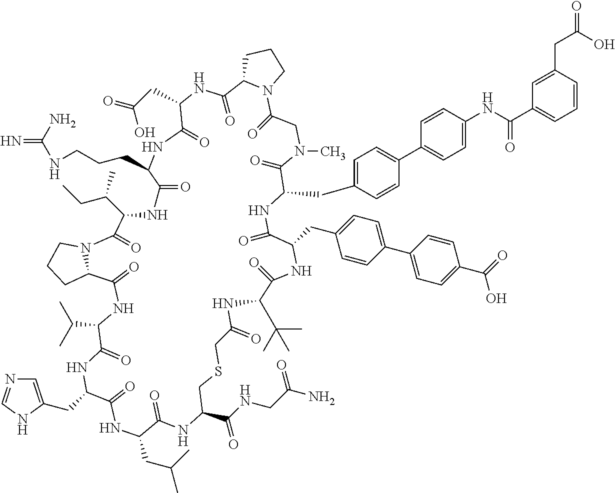 [SMILES: CC[C@H](C)[C@@H]1NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4cccc(CC(=O)O)c4)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 575 was prepared on a 50 umol scale. It was purified by Preparative LC/MS. The yield of the product was 15.1 mg, and its estimated purity by LCMS analysis was 92.5%. Analysis condition 1: Retention time=1.1 min. ESI-MS(+) m/z [M+H]⁺: 1996.7.

### Preparation of Example 576

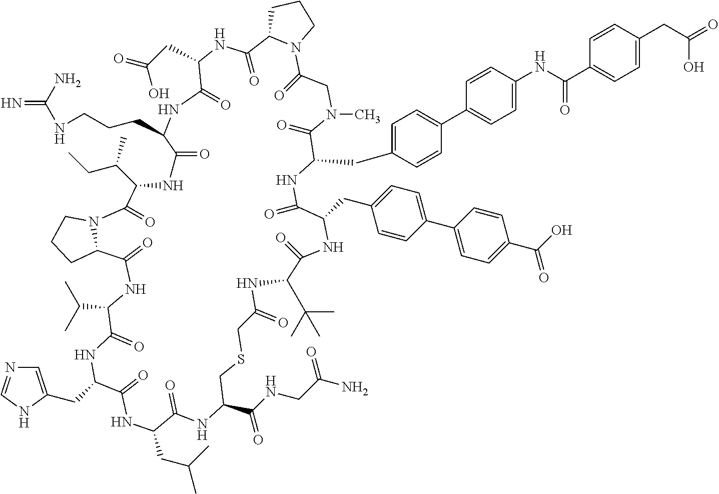 [SMILES: CC[C@H](C)[C@@H]1NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 576 was prepared on a 50 umol scale. It was purified by Preparative LC/MS. The yield of the product was 18.6 mg, and its estimated purity by LCMS analysis was 95.6%. Analysis condition 1: Retention time=1.08 min. ESI-MS(+) m/z [M+H]⁺: 1996.7.

### Preparation of Example 577

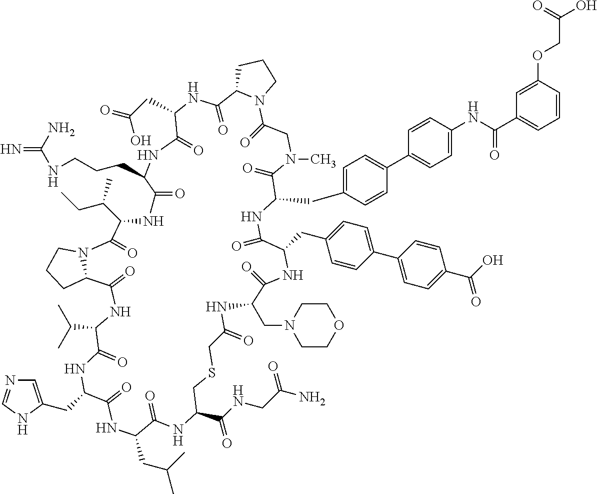 [SMILES: CC[C@H](C)[C@@H]1NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 577 was prepared on a 50 umol scale. It was purified by Preparative LC/MS. The yield of the product was 4.9 mg, and its estimated purity by LCMS analysis was 95.7%. Analysis condition 1: Retention time=0.99 min. ESI-MS(+) m/z [M+2H]²⁺: 1028.9.

### Preparation of Example 578

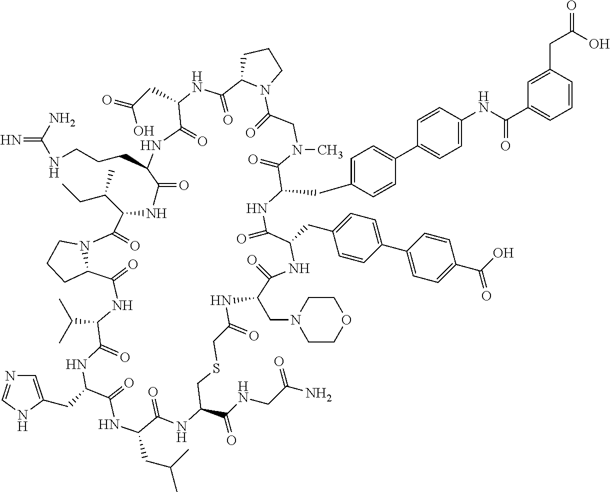 [SMILES: CC[C@H](C)[C@@H]1NC(=O)[C@@H](CCCNC(=N)N)NC(=O)[C@H](CC(=O)O)NC(=O)[C@@H]2CCCN2C(=O)CN(C)C(=O)[C@H](Cc2ccc(-c3ccc(NC(=O)c4cccc(CC(=O)O)c4)cc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 578 was prepared on a 50 umol scale. It was purified by Preparative LC/MS. The yield of the product was 24.1 mg, and its estimated purity by LCMS analysis was 94.1%. Analysis condition 1: Retention time=1.11 min. ESI-MS(+) m/z [M+2H]²⁺: 1020.9.

### Preparation of Example 579

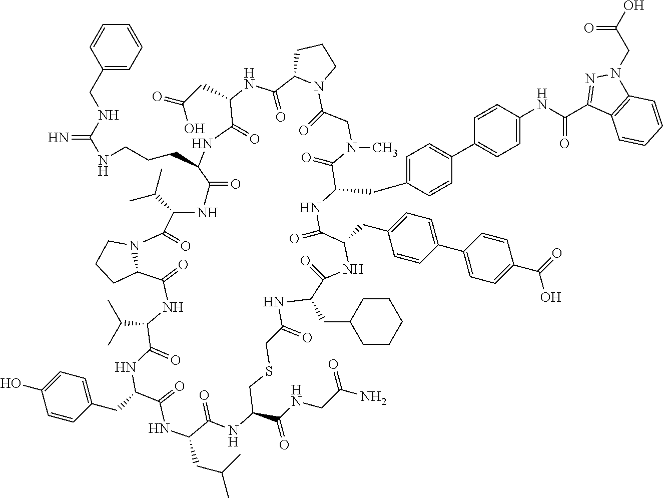 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)NCc2ccccc2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 579 was prepared on a 50 umol scale. It was purified by Preparative LC/MS. The yield of the product was 5 mg, and its estimated purity by LCMS analysis was 93.4%. Analysis condition 1: Retention time=1.27 min. ESI-MS(+) m/z [M+2H]²⁺: 1090.4.

### Preparation of Example 580

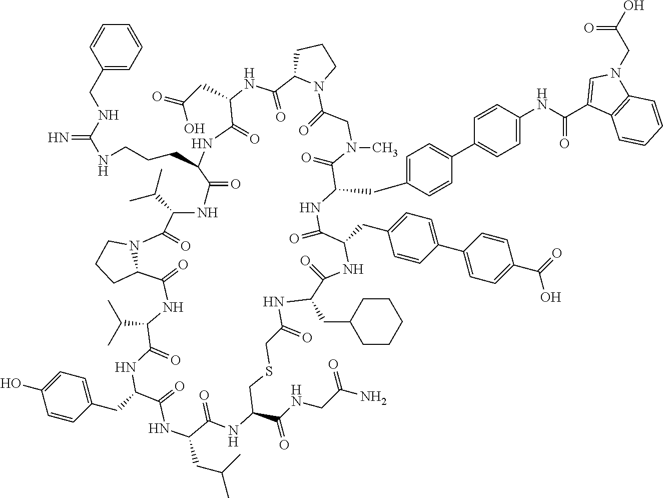 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)NCc2ccccc2)NC(=O)[C@H](CC(=O)O)NC(=O)[C@@H]2CCCN2C(=O)CN(C)C(=O)[C@H](Cc2ccc(-c3ccc(NC(=O)c4cn(CC(=O)O)c5ccccc45)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 580 was prepared on a 50 umol scale. It was purified by Preparative LC/MS. The yield of the product was 4.5 mg, and its estimated purity by LCMS analysis was 91%. Analysis condition 2: Retention time=1.64 min. ESI-MS(+) m/z [M+2H]²⁺: 1090.

### Preparation of Example 581

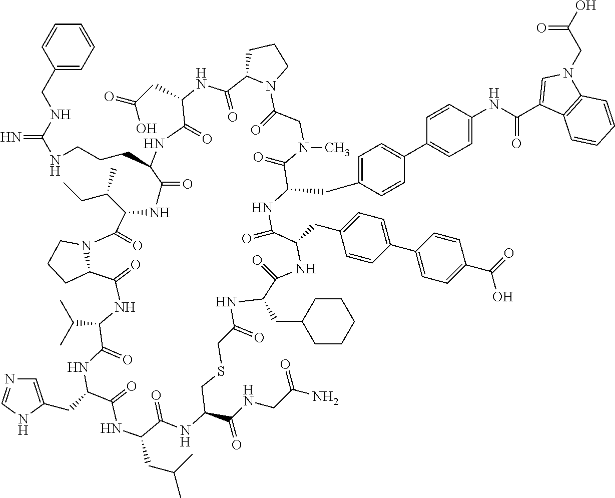 [SMILES: CC[C@H](C)[C@@H]1NC(=O)[C@@H](CCCNC(=N)NCc2ccccc2)NC(=O)[C@H](CC(=O)O)NC(=O)[C@@H]2CCCN2C(=O)CN(C)C(=O)[C@H](Cc2ccc(-c3ccc(NC(=O)c4cn(CC(=O)O)c5ccccc45)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 581 was prepared on a 50 umol scale. It was purified by Preparative LC/MS. The yield of the product was 6.3 mg, and its estimated purity by LCMS analysis was 90.8%. Analysis condition 1: Retention time=1.29 min. ESI-MS(+) m/z [M+2H]²⁺: 1104.4.

### Preparation of Example 582

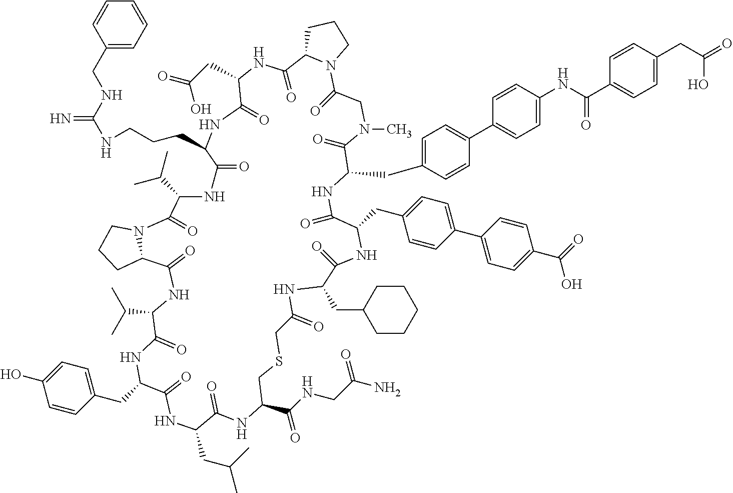 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)NCc2ccccc2)NC(=O)[C@H](CC(=O)O)NC(=O)[C@@H]2CCCN2C(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 582 was prepared on a 50 umol scale. It was purified by Preparative LC/MS. The yield of the product was 3.3 mg, and its estimated purity by LCMS analysis was 96%. Analysis condition 1: Retention time=1.11 min. ESI-MS(+) m/z [M+2H]²⁺: 1070.4.

### Preparation of Example 583

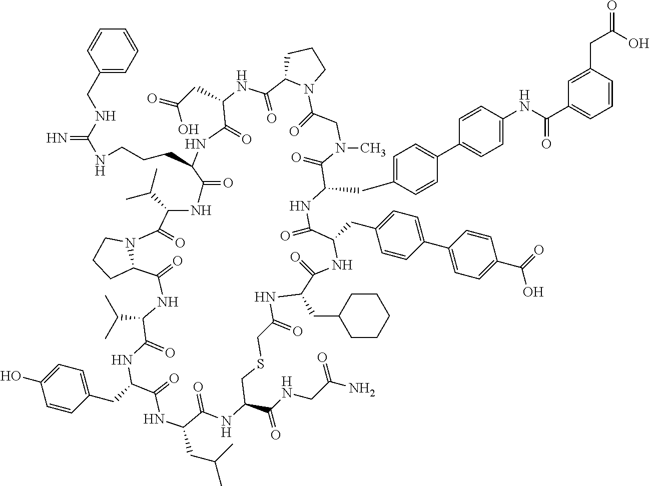 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)NCc2ccccc2)NC(=O)[C@H](CC(=O)O)NC(=O)[C@@H]2CCCN2C(=O)CN(C)C(=O)[C@H](Cc2ccc(-c3ccc(NC(=O)c4cccc(CC(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 583 was prepared on a 50 umol scale. It was purified by Preparative LC/MS. The yield of the product was 6.9 mg, and its estimated purity by LCMS analysis was 92.3%. Analysis condition 2: Retention time=1.59 min. ESI-MS(+) m/z [M+2H]²⁺: 1070.3.

### Preparation of Example 584

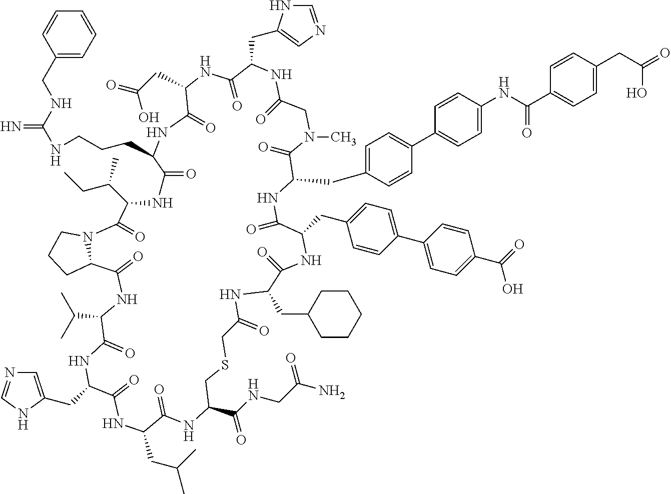 [SMILES: CC[C@H](C)[C@@H]1NC(=O)[C@@H](CCCNC(=N)NCc2ccccc2)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 584 was prepared on a 50 umol scale. It was purified by Preparative LC/MS. The yield of the product was 3.4 mg, and its estimated purity by LCMS analysis was 90.3%. Analysis condition 2: Retention time=1.37 min. ESI-MS(+) m/z [M+2H]²⁺: 1084.2.

### Preparation of Example 585

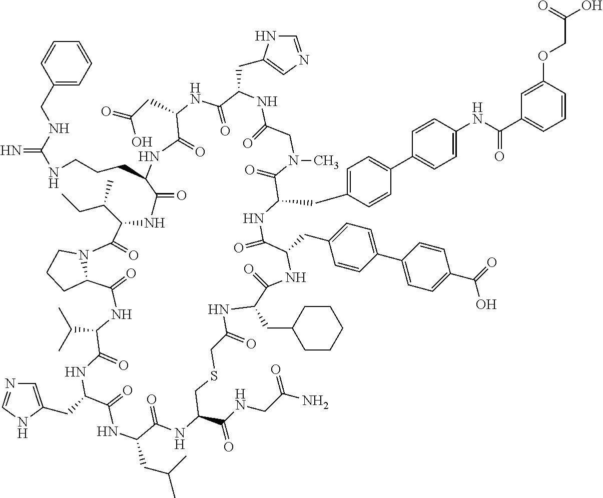 [SMILES: CC[C@H](C)[C@@H]1NC(=O)[C@@H](CCCNC(=N)NCc2ccccc2)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 585 was prepared on a 50 umol scale. It was purified by Preparative LC/MS. The yield of the product was 4.5 mg, and its estimated purity by LCMS analysis was 91.5%. Analysis condition 2: Retention time=1.43 min. ESI-MS(+) m/z [M+2H]²⁺: 1092.4.

### Preparation of Example 586

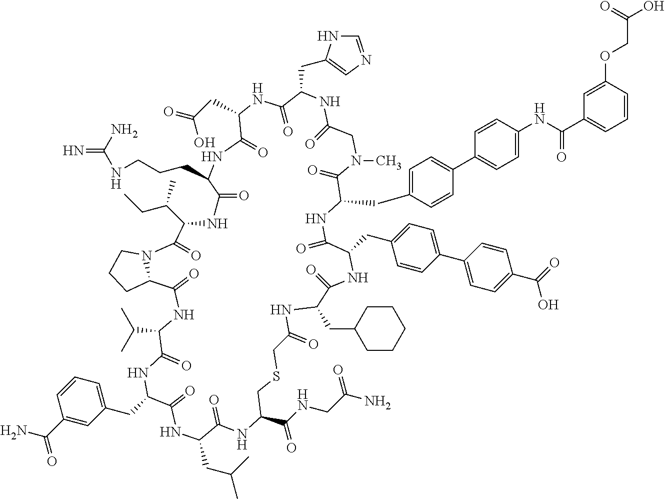 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cccc(C(N)=O)c2)NC(=O)[C@H](C(C)C)NC(=O)[C@@H]2CCCN2C1=O]

Example 586 was prepared on a 50 umol scale. It was purified by Preparative LC/MS. The yield of the product was 14.9 mg, and its estimated purity by LCMS analysis was 90.7%. Analysis condition 1: Retention time=1.17 min. ESI-MS(+) m/z [M+2H]²⁺: 1073.6.

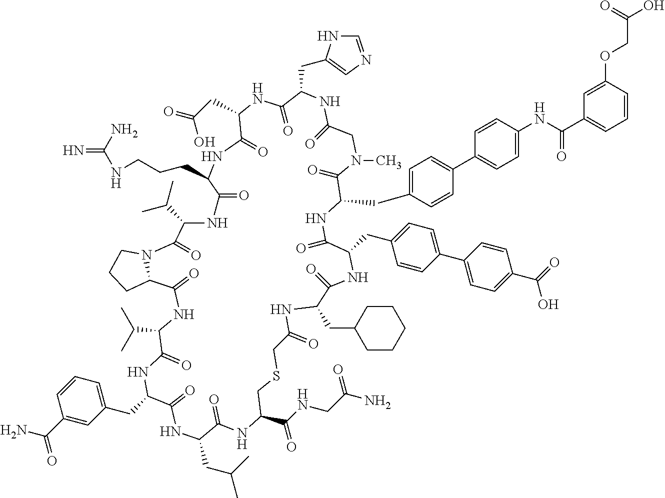 [SMILES: CC(C)C[C@@H]1NC(=O)[C@H](Cc2cccc(C(N)=O)c2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 587 was prepared on a 50 umol scale. It was purified by Preparative LC/MS. The yield of the product was 7.7 mg, and its estimated purity by LCMS analysis was 95.8%. Analysis condition 1: Retention time=1.15 min. ESI-MS(+) m/z [M+2H]²⁺: 1066.6.

### Preparation of Example 588

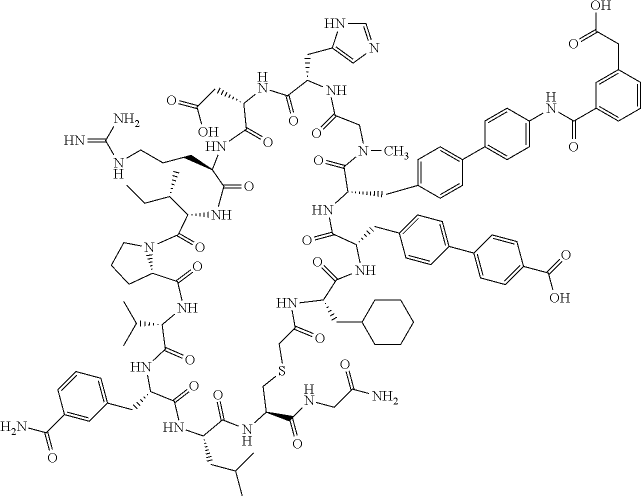 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cccc(C(N)=O)c2)NC(=O)[C@H](C(C)C)NC(=O)[C@@H]2CCCN2C1=O]

Example 588 was prepared on a 50 umol scale. It was purified by Preparative LC/MS. The yield of the product was 7.3 mg, and its estimated purity by LCMS analysis was 96%. Analysis condition 2: Retention time=1.46 min. ESI-MS(+) m/z [M+2H]²⁺: 1065.6.

### Preparation of Example 589

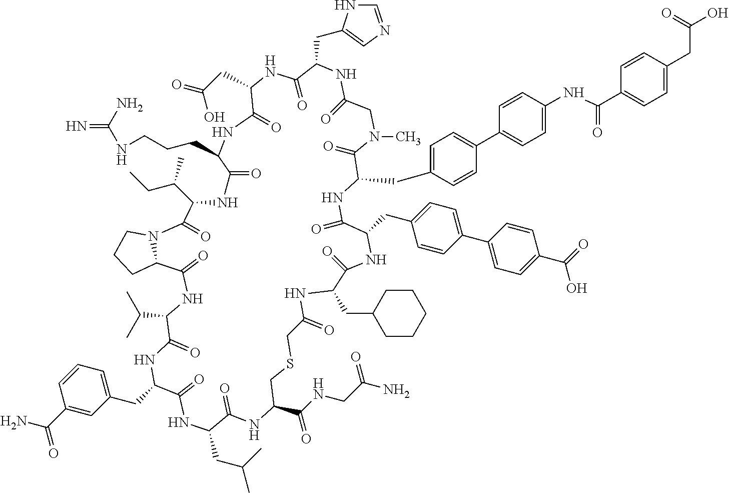 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cccc(C(N)=O)c2)NC(=O)[C@H](C(C)C)NC(=O)[C@@H]2CCCN2C1=O]

Example 589 was prepared on a 50 umol scale. It was purified by Preparative LC/MS. The yield of the product was 6.7 mg, and its estimated purity by LCMS analysis was 93.3%. Analysis condition 1: Retention time=1.14 min. ESI-MS(+) m/z [M+2H]²⁺: 1066.3.

### Preparation of Example 590

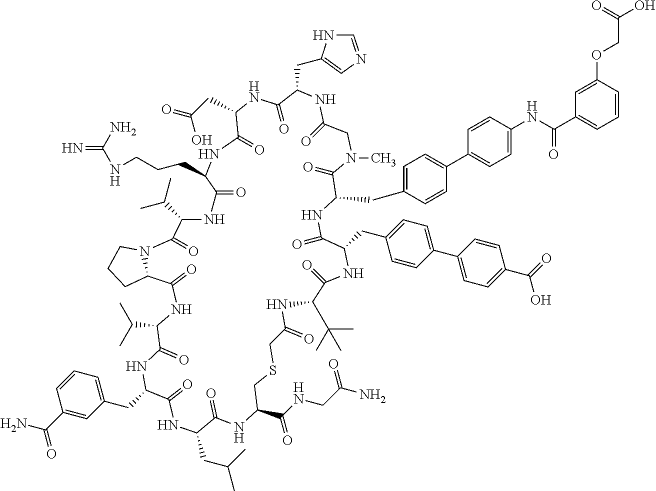 [SMILES: CC(C)C[C@@H]1NC(=O)[C@H](Cc2cccc(C(N)=O)c2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC1=O]

Example 590 was prepared on a 50 umol scale. It was purified by Preparative LC/MS. The yield of the product was 26.3 mg, and its estimated purity by LCMS analysis was 91.7%. Analysis condition 1: Retention time=1.07 min. ESI-MS(+) m/z [M+2H]²⁺: 1046.8.

### Preparation of Example 591

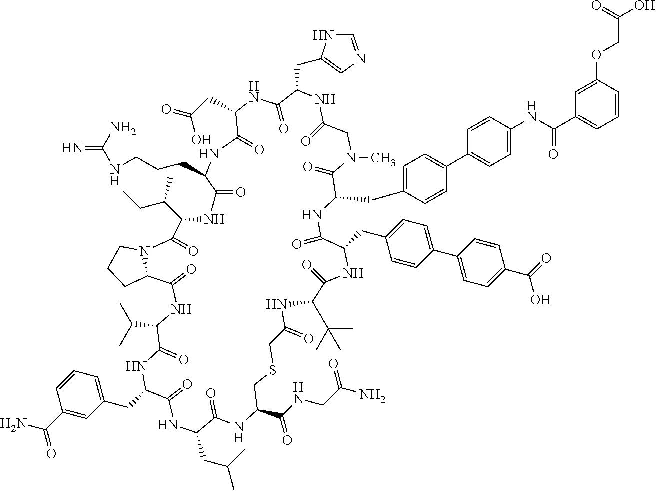 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC(=O)[C@H](CC(C)C)NC(=O)[C@H](Cc2cccc(C(N)=O)c2)NC(=O)[C@H](C(C)C)NC(=O)[C@@H]2CCCN2C1=O]

Example 591 was prepared on a 50 umol scale. It was purified by Preparative LC/MS. The yield of the product was 2 mg, and its estimated purity by LCMS analysis was 92.9%. Analysis condition 1: Retention time=1.09 min. ESI-MS(+) m/z [M+2H]²⁺: 1053.5.

### Preparation of Example 592

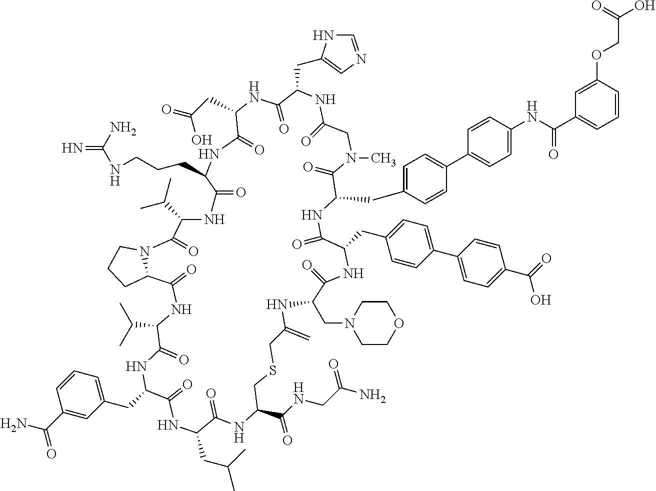 [SMILES: C=C1CSC[C@@H](C(=O)NCC(N)=O)NC(=O)[C@H](CC(C)C)NC(=O)[C@H](Cc2cccc(C(N)=O)c2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccc(C(=O)O)cc3)cc2)NC(=O)[C@H](CN2CCOCC2)N1]

Example 592 was prepared on a 50 umol scale. It was purified by Preparative LC/MS. The yield of the product was 17.4 mg, and its estimated purity by LCMS analysis was 99.4%. Analysis condition 1: Retention time=1.1 min. ESI-MS(+) m/z [M+2H]²⁺: 1068.2.

### Preparation of Example 593

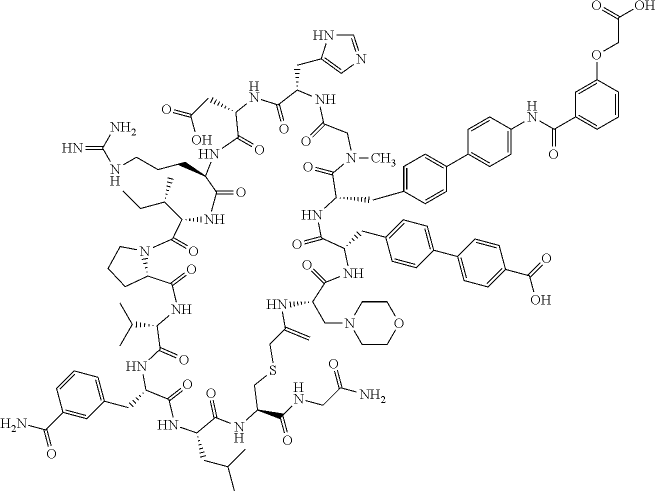 [SMILES: C=C1CSC[C@@H](C(=O)NCC(N)=O)NC(=O)[C@H](CC(C)C)NC(=O)[C@H](Cc2cccc(C(N)=O)c2)NC(=O)[C@H](C(C)C)NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccc(C(=O)O)cc3)cc2)NC(=O)[C@H](CN2CCOCC2)N1]

Example 593 was prepared on a 50 umol scale. It was purified by Preparative LC/MS. The yield of the product was 11.7 mg, and its estimated purity by LCMS analysis was 100%. Analysis condition 1: Retention time=1.06 min. ESI-MS(+) m/z [M+2H]²⁺: 1075.5.

### Preparation of Example 594

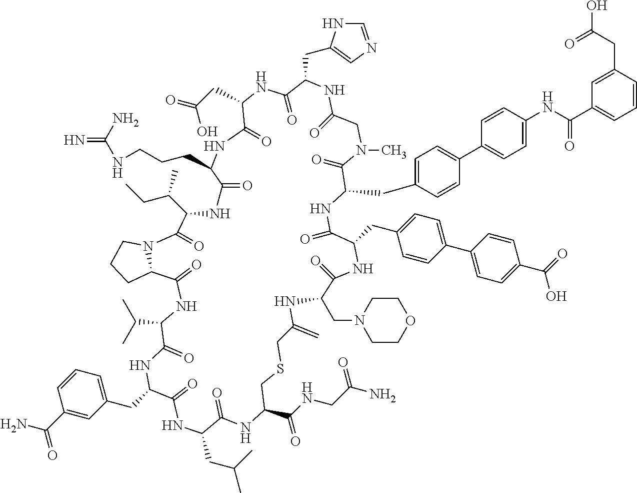 [SMILES: C=C1CSC[C@@H](C(=O)NCC(N)=O)NC(=O)[C@H](CC(C)C)NC(=O)[C@H](Cc2cccc(C(N)=O)c2)NC(=O)[C@H](C(C)C)NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccc(C(=O)O)cc3)cc2)NC(=O)[C@H](CN2CCOCC2)N1]

Example 594 was prepared on a 50 umol scale. It was purified by Preparative LC/MS. The yield of the product was 10.1 mg, and its estimated purity by LCMS analysis was 100%. Analysis condition 1: Retention time=1.12 min. ESI-MS(+) m/z [M+2H]²⁺: 1067.1.

### Preparation of Example 595

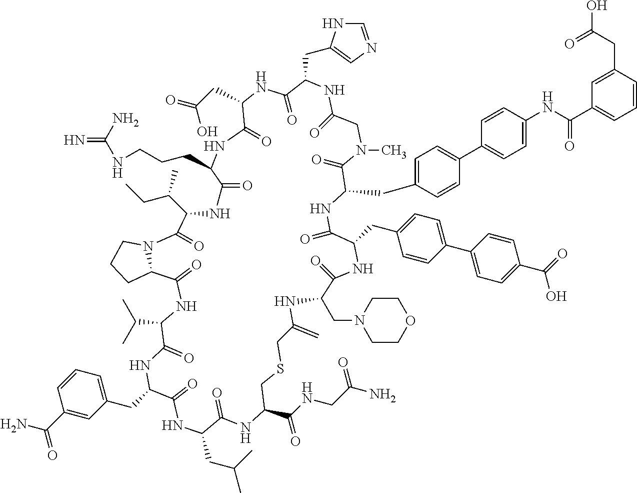 [SMILES: C=C1CSC[C@@H](C(=O)NCC(N)=O)NC(=O)[C@H](CC(C)C)NC(=O)[C@H](Cc2cccc(C(N)=O)c2)NC(=O)[C@H](C(C)C)NC(=O)[C@@H]2CCCN2C(=O)[C@H]([C@@H](C)CC)NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccc(C(=O)O)cc3)cc2)NC(=O)[C@H](CN2CCOCC2)N1]

Example 595 was prepared on a 50 umol scale. It was purified by Preparative LC/MS. The yield of the product was 10 mg, and its estimated purity by LCMS analysis was 100%. Analysis condition 1: Retention time=1.11 min. ESI-MS(+) m/z [M+2H]²⁺: 1067.1.

### Preparation of Example 596

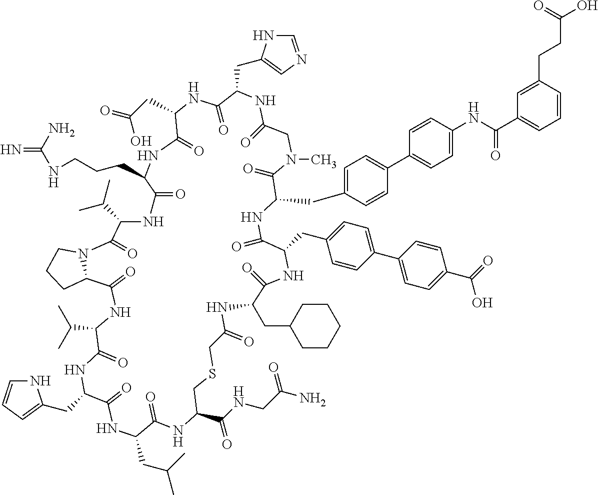 [SMILES: CC(C)C[C@@H]1NC(=O)[C@H](Cc2ccc[nH]2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(CCC(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 596 was prepared on a 50 umol scale. It was purified by Preparative LC/MS. The yield of the product was 3.7 mg, and its estimated purity by LCMS analysis was 91%. Analysis condition 1: Retention time=1.09 min. ESI-MS(+) m/z [M+2H]²⁺: 1040.8.

### Preparation of Example 597

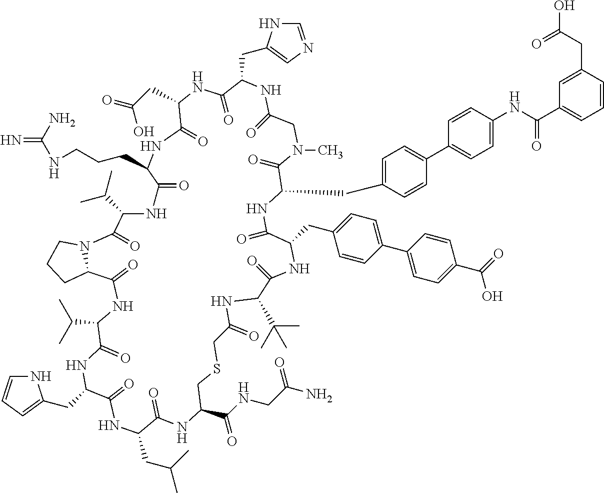 [SMILES: CC(C)C[C@@H]1NC(=O)[C@H](Cc2ccc[nH]2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC1=O]

Example 597 was prepared on a 50 umol scale. It was purified by Preparative LC/MS. The yield of the product was 3.3 mg, and its estimated purity by LCMS analysis was 92.7%. Analysis condition 1: Retention time=1.03 min. ESI-MS(+) m/z [M+2H]²⁺: 1012.8.

### Preparation of Example 598

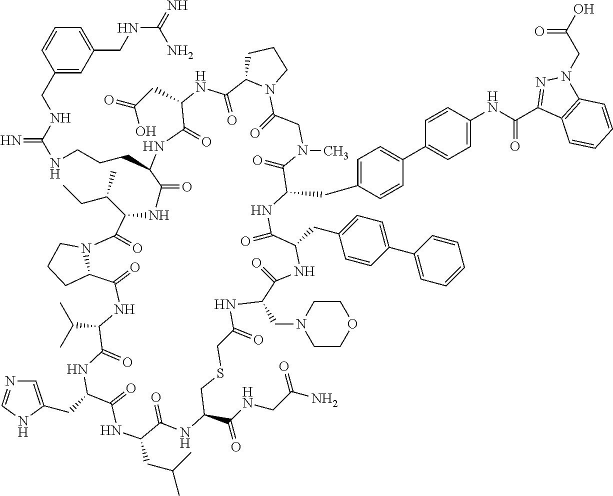 [SMILES: CC[C@H](C)[C@@H]1NC(=O)[C@@H](CCCNC(=N)NCc2cccc(CNC(=N)N)c2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 598 was prepared on a 100 umol scale. It was purified by Preparative LC/MS. The yield of the product was 18.2 mg, and its estimated purity by LCMS analysis was 100%. Analysis condition 1: Retention time=1.41 min. ESI-MS(+) m/z [M+2H]²⁺: 1099.3.

### Preparation of Example 599

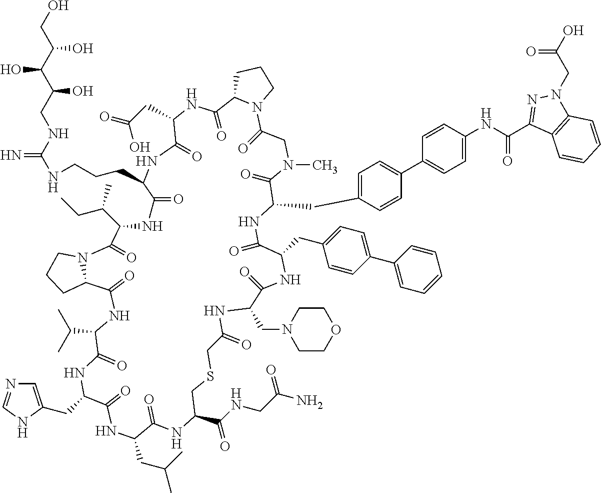 [SMILES: CC[C@H](C)[C@@H]1NC(=O)[C@@H](CCCNC(=N)NC[C@H](O)[C@@H](O)[C@@H](O)CO)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 599 was prepared on a 50 umol scale. It was purified by Preparative LC/MS. The yield of the product was 10.6 mg, and its estimated purity by LCMS analysis was 86.4%. Analysis condition 1: Retention time=1.34 min. ESI-MS(+) m/z [M+2H]²⁺: 1085.8.

### Preparation of Example 600

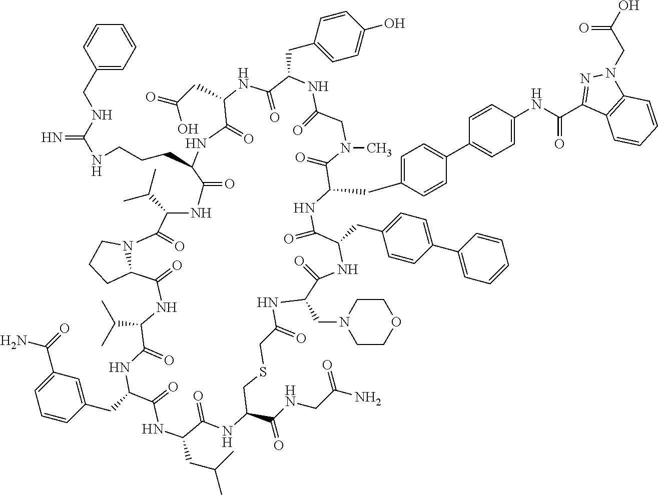 [SMILES: CC(C)C[C@@H]1NC(=O)[C@H](Cc2cccc(C(N)=O)c2)NC(=O)[C@H](C(C)C)NC(=O)[C@@H]2CCCN2C(=O)[C@H](C(C)C)NC(=O)[C@@H](CCCNC(=N)NCc2ccccc2)NC(=O)[C@H](CC(=O)O)NC(=O)[C@H](Cc2ccc(O)cc2)NC(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 600 was prepared on a 50 umol scale. It was purified by Preparative LC/MS. The yield of the product was 7.6 mg, and its estimated purity by LCMS analysis was 89.9%. Analysis condition 1: Retention time=1.53 min. ESI-MS(+) m/z [M+2H]²⁺: 1116.3.

### Preparation of Example 601

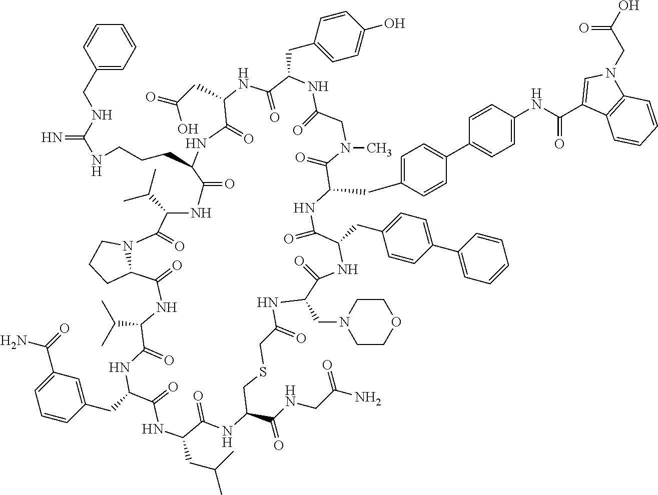 [SMILES: CC(C)C[C@@H]1NC(=O)[C@H](Cc2cccc(C(N)=O)c2)NC(=O)[C@H](C(C)C)NC(=O)[C@@H]2CCCN2C(=O)[C@H](C(C)C)NC(=O)[C@@H](CCCNC(=N)NCc2ccccc2)NC(=O)[C@H](CC(=O)O)NC(=O)[C@H](Cc2ccc(O)cc2)NC(=O)CN(C)C(=O)[C@H](Cc2ccc(-c3ccc(NC(=O)c4cn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 601 was prepared on a 50 umol scale. It was purified by Preparative LC/MS. The yield of the product was 4.2 mg, and its estimated purity by LCMS analysis was 91.8%. Analysis condition 2: Retention time=1.58 min. ESI-MS(+) m/z [M+2H]²⁺: 1115.9.

### Preparation of Example 602

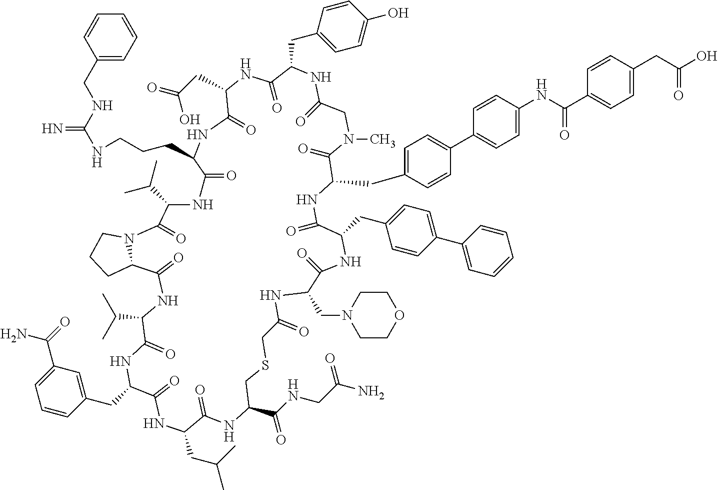 [SMILES: CC(C)C[C@@H]1NC(=O)[C@H](Cc2cccc(C(N)=O)c2)NC(=O)[C@H](C(C)C)NC(=O)[C@@H]2CCCN2C(=O)[C@H](C(C)C)NC(=O)[C@@H](CCCNC(=N)NCc2ccccc2)NC(=O)[C@H](CC(=O)O)NC(=O)[C@H](Cc2ccc(O)cc2)NC(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 602 was prepared on a 50 umol scale. It was purified by Preparative LC/MS. The yield of the product was 9.5 mg, and its estimated purity by LCMS analysis was 90.4%. Analysis condition 1: Retention time=1.47 min. ESI-MS(+) m/z [M+2H]²⁺: 1096.4.

### Preparation of Example 603

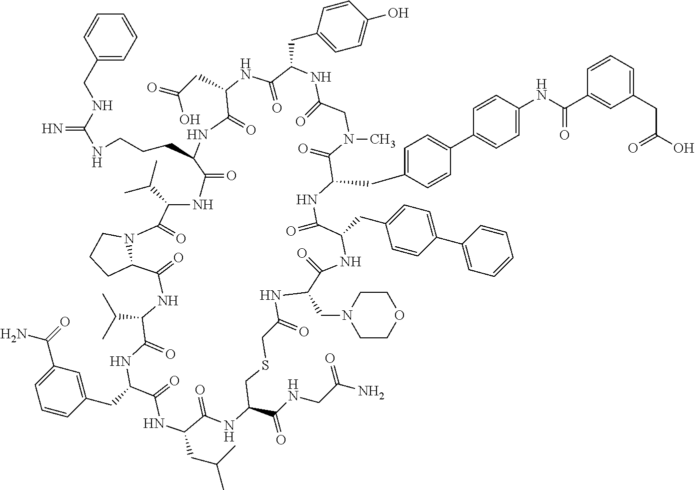 [SMILES: CC(C)C[C@@H]1NC(=O)[C@H](Cc2cccc(C(N)=O)c2)NC(=O)[C@H](C(C)C)NC(=O)[C@@H]2CCCN2C(=O)[C@H](C(C)C)NC(=O)[C@@H](CCCNC(=N)NCc2ccccc2)NC(=O)[C@H](CC(=O)O)NC(=O)[C@H](Cc2ccc(O)cc2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 603 was prepared on a 50 umol scale. It was purified by Preparative LC/MS. The yield of the product was 8 mg, and its estimated purity by LCMS analysis was 95.5%. Analysis condition 1: Retention time=1.49 min. ESI-MS(+) m/z [M+2H]²⁺: 1096.1.

### Preparation of Example 604

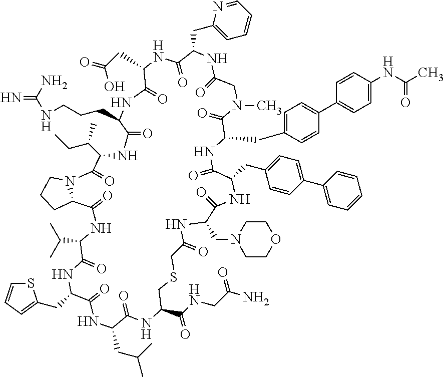 [SMILES: CC[C@H](C)[C@@H]1NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(C)=O)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2cccs2)NC(=O)[C@H](C(C)C)NC(=O)[C@@H]2CCCN2C1=O]

Example 604 was prepared on a 45 umol scale. It was purified by Preparative LC/MS. The yield of the product was 4.5 mg, and its estimated purity by LCMS analysis was 95.5%. Analysis condition 2: Retention time=1.46 min. ESI-MS(+) m/z [M+2H]²⁺: 972.3.

### Preparation of Example 605

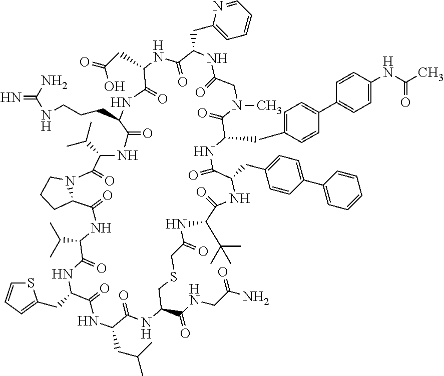 [SMILES: CC(=O)Nc1ccc(-c2ccc(C[C@@H]3NC(=O)[C@H](Cc4ccc(-c5ccccc5)cc4)NC(=O)[C@H](C(C)(C)C)NC(=O)CSC[C@@H](C(=O)NCC(N)=O)NC(=O)[C@H](CC(C)C)NC(=O)[C@H](Cc4cccs4)NC(=O)[C@H](C(C)C)NC(=O)[C@@H]4CCCN4C(=O)[C@H](C(C)C)NC(=O)[C@@H](CCCNC(=N)N)NC(=O)[C@H](CC(=O)O)NC(=O)[C@H](Cc4ccccn4)NC(=O)CN(C)C3=O)cc2)cc1]

Example 605 was prepared on a 50 Preparative LC/MS. The yield of the product was 1.3 mg, and its estimated purity by LCMS analysis was 98.9%. Analysis condition 1: Retention time=1.5 min. ESI-MS(+) m/z [M+H]⁺: 1885.6.

### Preparation of Example 606

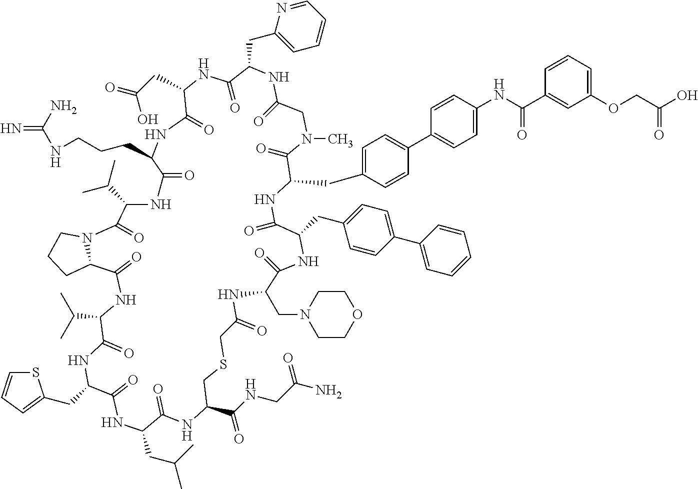 [SMILES: CC(C)C[C@@H]1NC(=O)[C@H](Cc2cccs2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 606 was prepared on a 50 umol scale. It was purified by Preparative LC/MS. The yield of the product was 5 mg, and its estimated purity by LCMS analysis was 100%. Analysis condition 1: Retention time=1.43 min. ESI-MS(+) m/z [M+2H] 2+: 1033.6.

### Preparation of Example 607

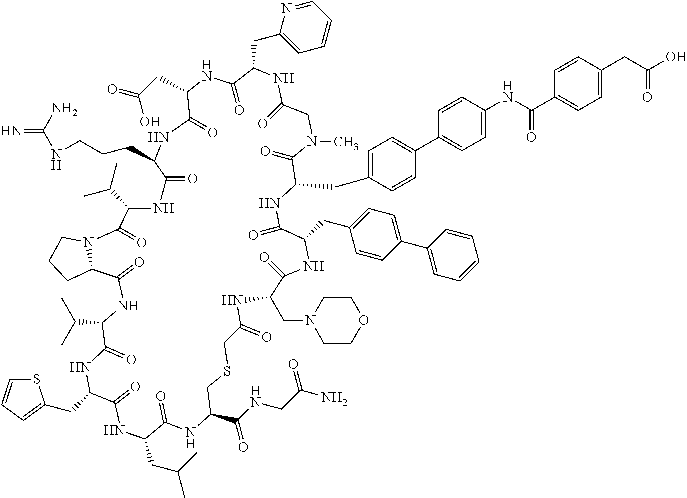 [SMILES: CC(C)C[C@@H]1NC(=O)[C@H](Cc2cccs2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 607 was prepared on a 50 Preparative LC/MS. The yield of the product was 5 mg, and its estimated purity by LCMS analysis was 100%. Analysis condition 1: Retention time=1.47 min. ESI-MS(+) m/z [M+2H]²⁺: 1025.3.

### Preparation of Example 608

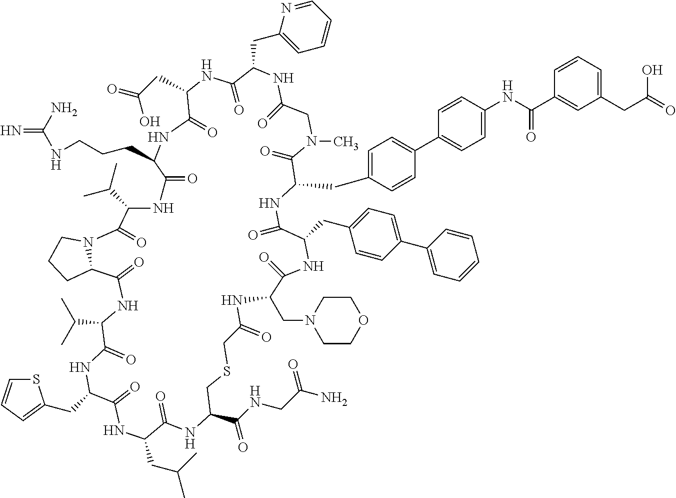 [SMILES: CC(C)C[C@@H]1NC(=O)[C@H](Cc2cccs2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 608 was prepared on a 50 umol scale. It was purified by Preparative LC/MS. The yield of the product was 5.7 mg, and its estimated purity by LCMS analysis was 100%. Analysis condition 1: Retention time=1.48 min. ESI-MS(+) m/z [M+2H]²⁺: 1025.4.

### Preparation of Example 609

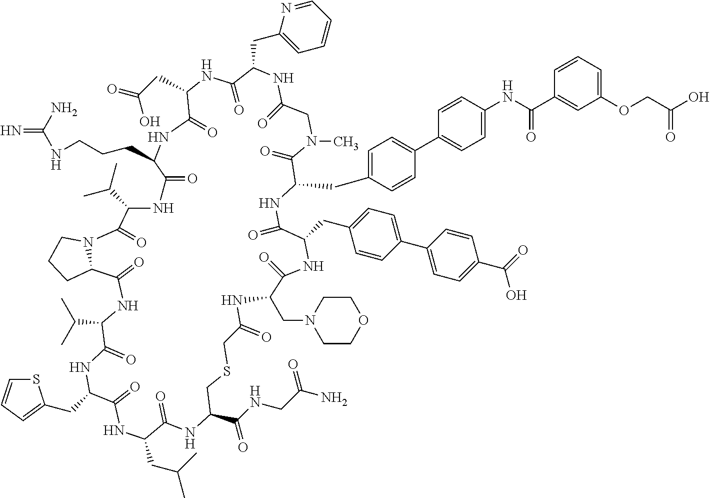 [SMILES: CC(C)C[C@@H]1NC(=O)[C@H](Cc2cccs2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC1=O]

Example 609 was prepared on a 50 Preparative LC/MS. The yield of the product was 8.3 mg, and its estimated purity by LCMS analysis was 91.8%. Analysis condition 2: Retention time=1.37 min. ESI-MS(+) m/z [M+2H]²⁺: 1055.4.

### Preparation of Example 610

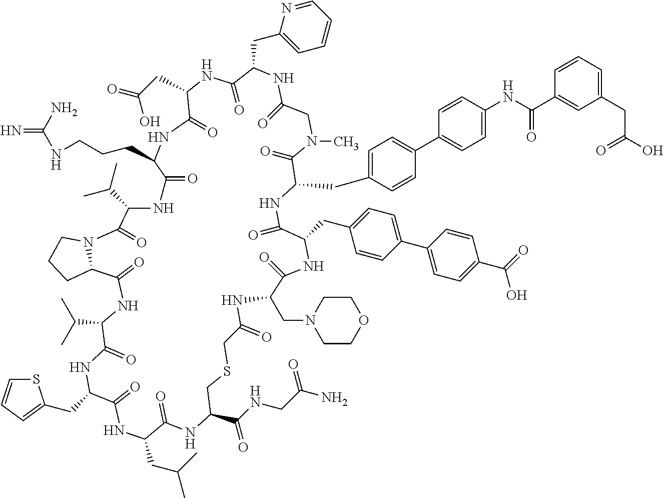 [SMILES: CC(C)C[C@@H]1NC(=O)[C@H](Cc2cccs2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC1=O]

Example 610 was prepared on a 50 umol scale. It was purified by Preparative LC/MS. The yield of the product was 5.5 mg, and its estimated purity by LCMS analysis was 91%. Analysis condition 2: Retention time=1.38 min. ESI-MS(+) m/z [M+2H]²⁺: 1047.3.

### Preparation of Example 611

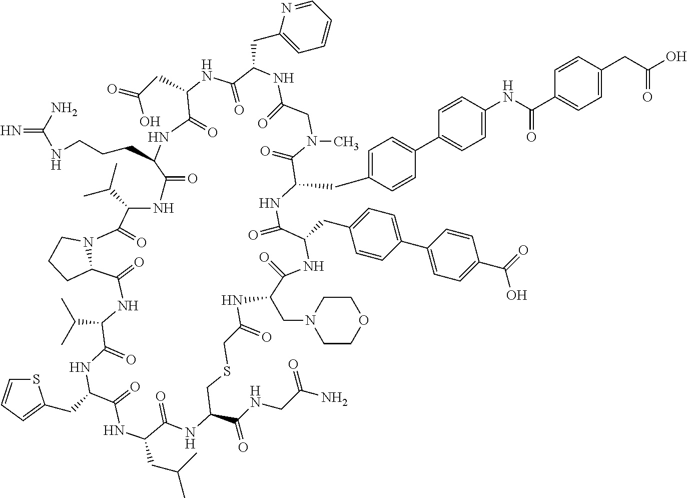 [SMILES: CC(C)C[C@@H]1NC(=O)[C@H](Cc2cccs2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccc(C(=O)O)cc3)cc2)NC(=O)[C@H](CN2CCOCC2)NC(=O)CSC[C@@H](C(=O)NCC(N)=O)NC1=O]

Example 611 was prepared on a 50 Preparative LC/MS. The yield of the product was 2.5 mg, and its estimated purity by LCMS analysis was 91.9%. Analysis condition 1: Retention time=1.18 min. ESI-MS(+) m/z [M+2H]²⁺: 1047.

### Preparation of Example 612

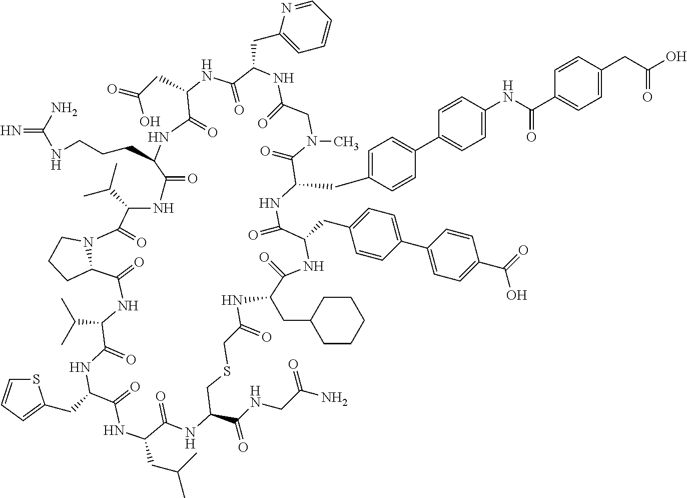 [SMILES: CC(C)C[C@@H]1NC(=O)[C@H](Cc2cccs2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 612 was prepared on a 50 umol scale. It was purified by Preparative LC/MS. The yield of the product was 1.7 mg, and its estimated purity by LCMS analysis was 91.2%. Analysis condition 1: Retention time=1.24 min. ESI-MS(+) m/z [M+2H]²⁺: 1045.9.

### Preparation of Example 613

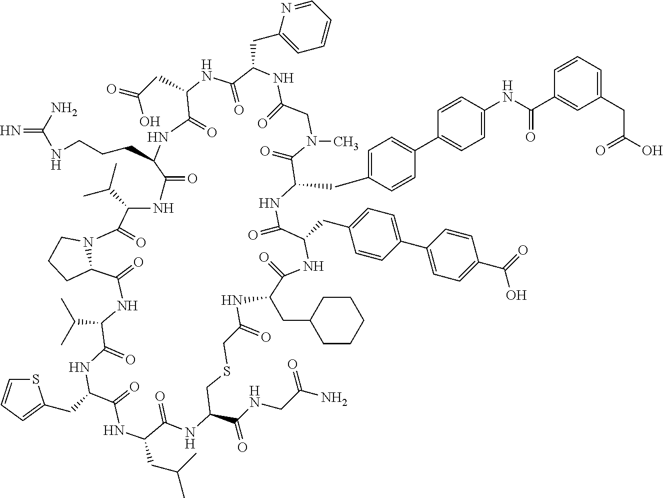 [SMILES: CC(C)C[C@@H]1NC(=O)[C@H](Cc2cccs2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 613 was prepared on a 50 umol scale. It was purified by Preparative LC/MS. The yield of the product was 3.1 mg, and its estimated purity by LCMS analysis was 92.4%. Analysis condition 1: Retention time=1.23 min. ESI-MS(+) m/z [M+2H]²⁺: 1046.

### Preparation of Example 614

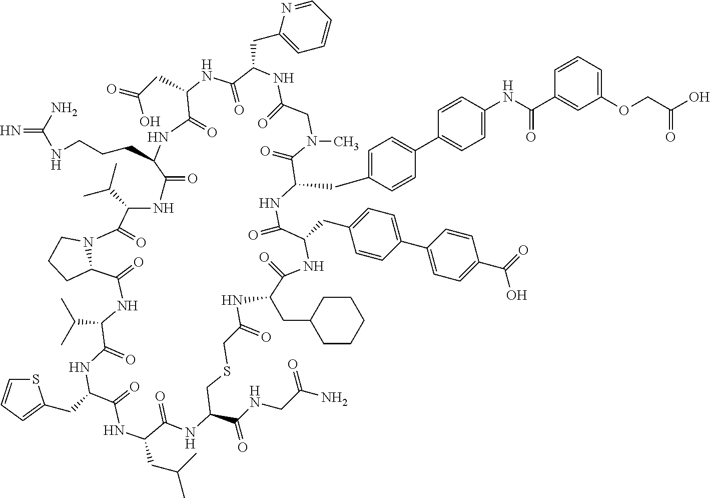 [SMILES: CC(C)C[C@@H]1NC(=O)[C@H](Cc2cccs2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC1=O]

Example 614 was prepared on a 50 umol scale. It was purified by Preparative LC/MS. The yield of the product was 3.4 mg, and its estimated purity by LCMS analysis was 92.1%. Analysis condition 1: Retention time=1.27 min. ESI-MS(+) m/z [M+2H]²⁺: 1053.8.

### Preparation of Example 615

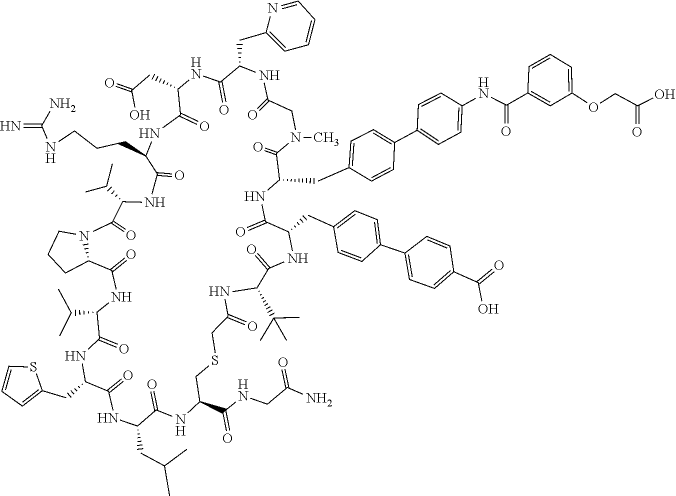 [SMILES: CC(C)C[C@@H]1NC(=O)[C@H](Cc2cccs2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccc(C(=O)O)cc3)cc2)NC(=O)[C@H](C(C)(C)C)NC(=O)CSC[C@@H](C(=O)NCC(N)=O)NC1=O]

Example 615 was prepared on a 50 Preparative LC/MS. The yield of the product was 1.7 mg, and its estimated purity by LCMS analysis was 95.4%. Analysis condition 1: Retention time=1.17 min. ESI-MS(+) m/z [M+2H]²⁺: 1034.

### Preparation of Example 616

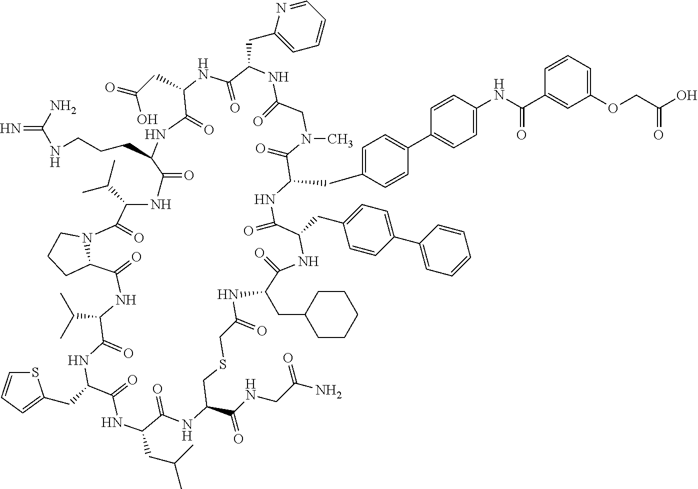 [SMILES: CC(C)C[C@@H]1NC(=O)[C@H](Cc2cccs2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC1=O]

Example 616 was prepared on a 50 umol scale. It was purified by Preparative LC/MS. The yield of the product was 2.9 mg, and its estimated purity by LCMS analysis was 91.1%. Analysis condition 1: Retention time=1.54 min. ESI-MS(+) m/z [M+2H]²⁺: 1031.8.

### Preparation of Example 617

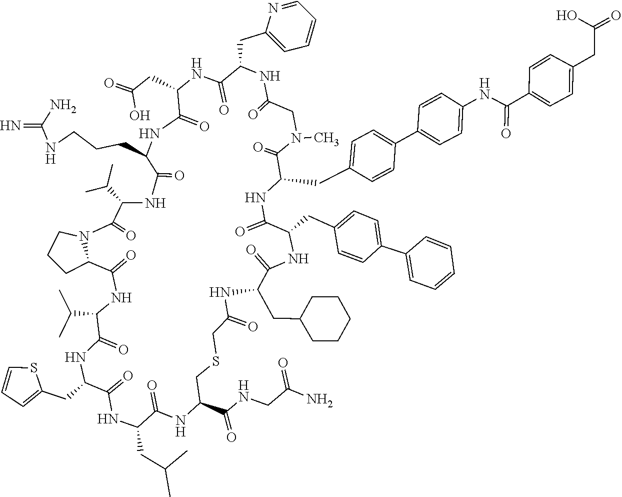 [SMILES: CC(C)C[C@@H]1NC(=O)[C@H](Cc2cccs2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC1=O]

Example 617 was prepared on a 50 Preparative LC/MS. The yield of the product was 5.8 mg, and its estimated purity by LCMS analysis was 95.9%. Analysis condition 2: Retention time=1.66 min. ESI-MS(+) m/z [M+2H]²⁺: 1023.7.

### Preparation of Example 618

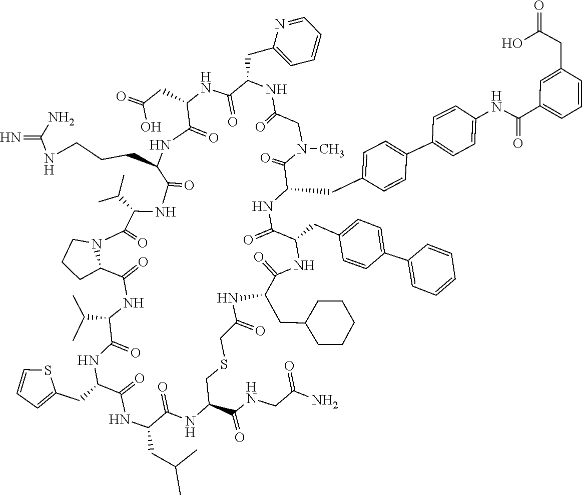 [SMILES: CC(C)C[C@@H]1NC(=O)[C@H](Cc2cccs2)NC(=O)[C@H](C(C)C)NC(=O)[C@@H]2CCCN2C(=O)[C@H](C(C)C)NC(=O)[C@@H](CCCNC(=N)N)NC(=O)[C@H](CC(=O)O)NC(=O)[C@H](Cc2ccccn2)NC(=O)CN(C)C(=O)[C@H](Cc2ccc(-c3ccc(NC(=O)c4cccc(CC(=O)O)c4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC1=O]

Example 618 was prepared on a 50 umol scale. It was purified by Preparative LC/MS. The yield of the product was 2.9 mg, and its estimated purity by LCMS analysis was 91.3%. Analysis condition 1: Retention time=1.55 min. ESI-MS(+) m/z [M+2H]²⁺: 1023.6.

### Preparation of Example 619

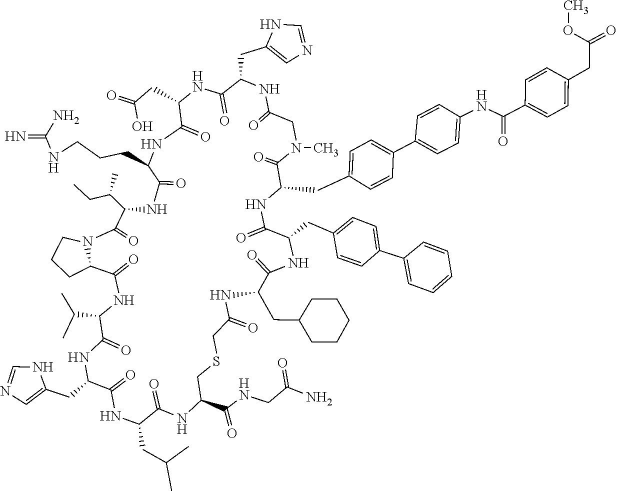 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(NC(=O)c4ccc(CC(=O)OC)cc4)cc3)cc2)NC(=O)[C@H](Cc2ccc(-c3ccccc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 619 was prepared on a 50 Preparative LC/MS. The yield of the product was 10.7 mg, and its estimated purity by LCMS analysis was 97.2%. Analysis condition 2: Retention time=1.51 min. ESI-MS(+) m/z [M+2H]²⁺: 1046.4.

### Preparation of Example 620

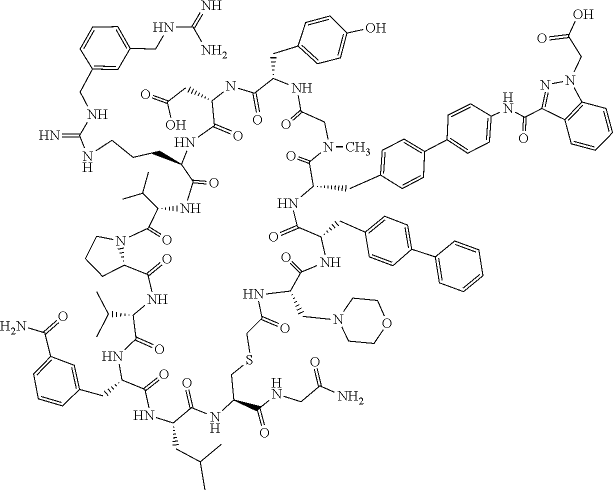

Example 620 was prepared on a 50 umol scale. It was purified by Preparative LC/MS. The yield of the product was 10.2 mg, and its estimated purity by LCMS analysis was 92.7%. Analysis condition 1: Retention time=1.38 min. ESI-MS(+) m/z [M+2H]²⁺: 1152.2.

### Preparation of Example 621

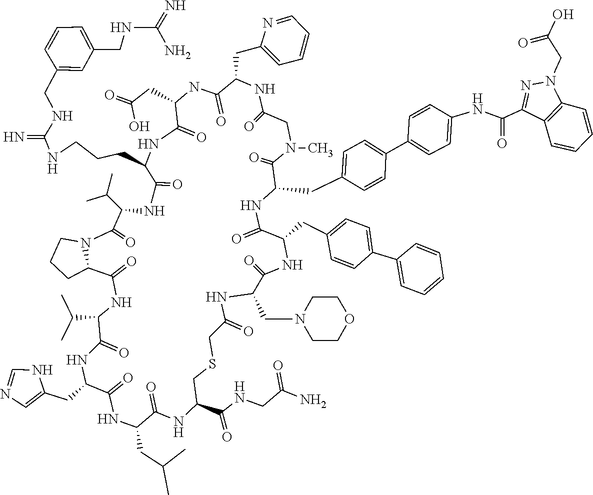

Example 621 was prepared on a 50 Preparative LC/MS. The yield of the product was 12.7 mg, and its estimated purity by LCMS analysis was 95.9%. Analysis condition 1: Retention time=1.5 min. ESI-MS(+) m/z [M+2H]²⁺: 1117.9.

### Preparation of Example 622

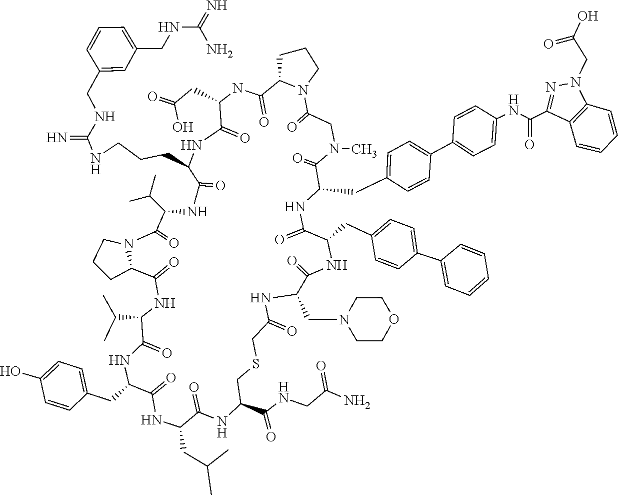

Example 622 was prepared on a 50 umol scale. It was purified by Preparative LC/MS. The yield of the product was 12.1 mg, and its estimated purity by LCMS analysis was 95.3%. Analysis condition 1: Retention time=1.43 min. ESI-MS(+) m/z [M+2H]²⁺: 1105.1.

### Preparation of Example 623

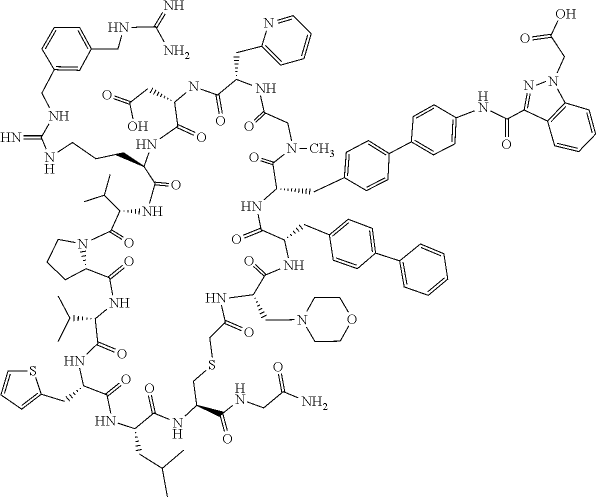

Example 623 was prepared on a 50 umol scale. It was purified by Preparative LC/MS. The yield of the product was 17.8 mg, and its estimated purity by LCMS analysis was 100%. Analysis condition 1: Retention time=1.48 min. ESI-MS(+) m/z [M+3H]³⁺: 751.1.

### Preparation of Example 624

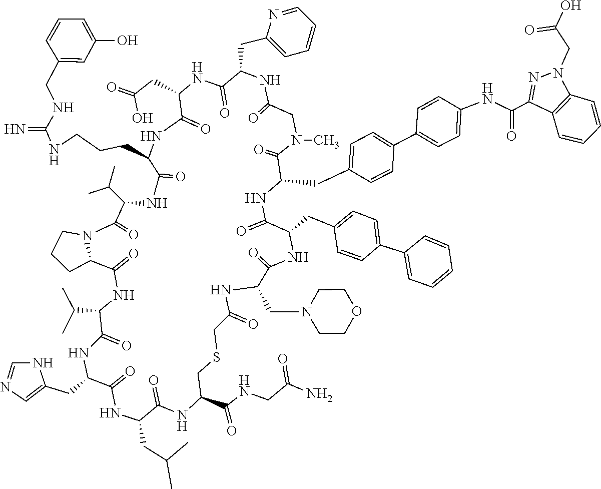 [SMILES: CC(C)C[C@@H]1NC(=O)[C@H](Cc2cnc[nH]2)NC(=O)[C@H](C(C)C)NC(=O)[C@@H]2CCCN2C(=O)[C@H](C(C)C)NC(=O)[C@@H](CCCNC(=N)NCc2cccc(O)c2)NC(=O)[C@H](CC(=O)O)NC(=O)[C@H](Cc2ccccn2)NC(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 624 was prepared on a 50 umol scale. It was purified by Preparative LC/MS. The yield of the product was 13.4 mg, and its estimated purity by LCMS analysis was 96.3%. Analysis condition 2: Retention time=1.43 min. ESI-MS(+) m/z [M+2H]²⁺: 1090.3.

### Preparation of Example 625

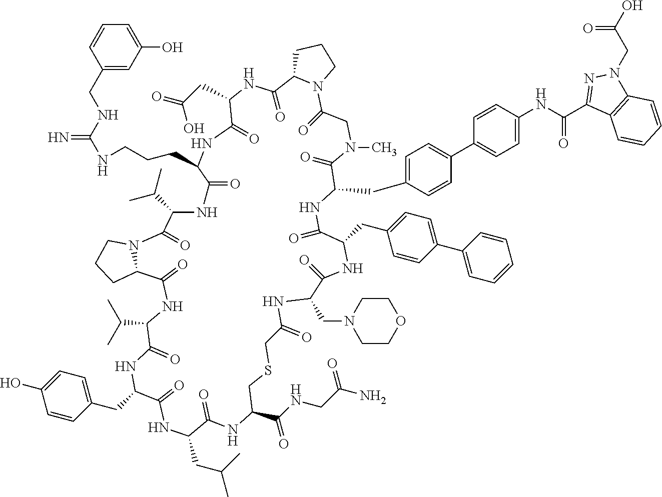 [SMILES: CC(C)C[C@@H]1NC(=O)[C@H](Cc2ccc(O)cc2)NC(=O)[C@H](C(C)C)NC(=O)[C@@H]2CCCN2C(=O)[C@H](C(C)C)NC(=O)[C@@H](CCCNC(=N)NCc2cccc(O)c2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 625 was prepared on a 50 Preparative LC/MS. The yield of the product was 11 mg, and its estimated purity by LCMS analysis was 94.5%. Analysis condition 1: Retention time=1.41 min. ESI-MS(+) m/z [M+2H]²⁺: 1077.8.

### Preparation of Example 626

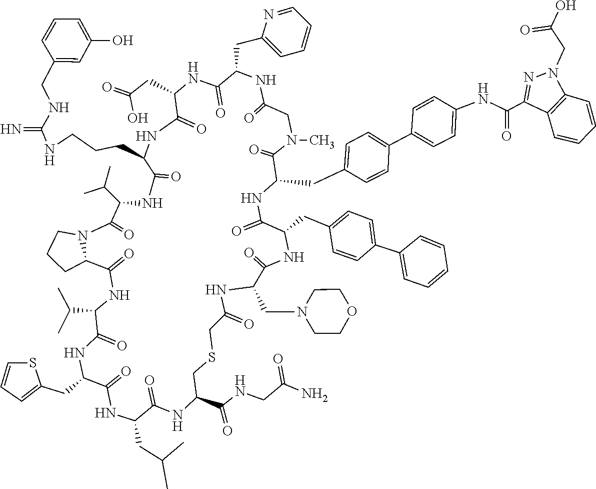 [SMILES: CC(C)C[C@@H]1NC(=O)[C@H](Cc2cccs2)NC(=O)[C@H](C(C)C)NC(=O)[C@@H]2CCCN2C(=O)[C@H](C(C)C)NC(=O)[C@@H](CCCNC(=N)NCc2cccc(O)c2)NC(=O)[C@H](CC(=O)O)NC(=O)[C@H](Cc2ccccn2)NC(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 626 was prepared on a 50 umol scale. It was purified by Preparative LC/MS. The yield of the product was 21.6 mg, and its estimated purity by LCMS analysis was 90%. Analysis condition 2: Retention time=1.58 min. ESI-MS(+) m/z [M+2H]²⁺: 1098.3.

### Preparation of Example 627

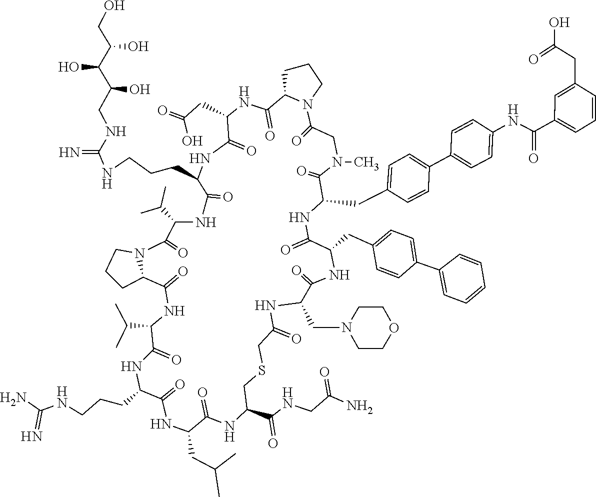 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NC[C@H](O)[C@@H](O)[C@@H](O)CO)NC(=O)[C@H](CC(=O)O)NC(=O)[C@@H]2CCCN2C(=O)CN(C)C(=O)[C@H](Cc2ccc(-c3ccc(NC(=O)c4cccc(CC(=O)O)c4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 627 was prepared on a 50 Preparative LC/MS. The yield of the product was 9 mg, and its estimated purity by LCMS analysis was 85.4%. Analysis condition 2: Retention time=1.29 min. ESI-MS(+) m/z [M+2H]²⁺: 1068.3.

### Preparation of Example 628

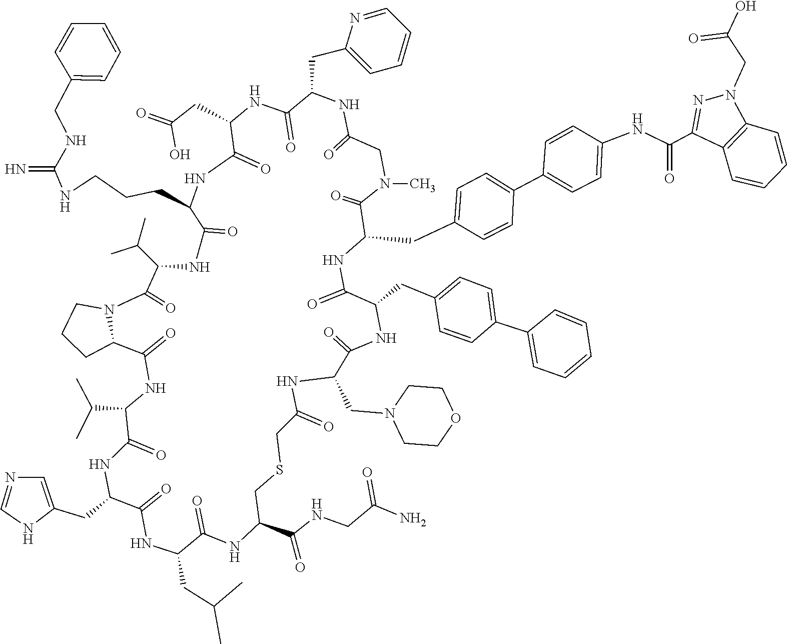 [SMILES: CC(C)C[C@@H]1NC(=O)[C@H](Cc2cnc[nH]2)NC(=O)[C@H](C(C)C)NC(=O)[C@@H]2CCCN2C(=O)[C@H](C(C)C)NC(=O)[C@@H](CCCNC(=N)NCc2ccccc2)NC(=O)[C@H](CC(=O)O)NC(=O)[C@H](Cc2ccccn2)NC(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 628 was prepared on a 50 umol scale. It was purified by Preparative LC/MS. The yield of the product was 2.6 mg, and its estimated purity by LCMS analysis was 95.1%. Analysis condition 1: Retention time=1.53 min. ESI-MS(+) m/z [M+2H]²⁺: 1082.1.

### Preparation of Example 629

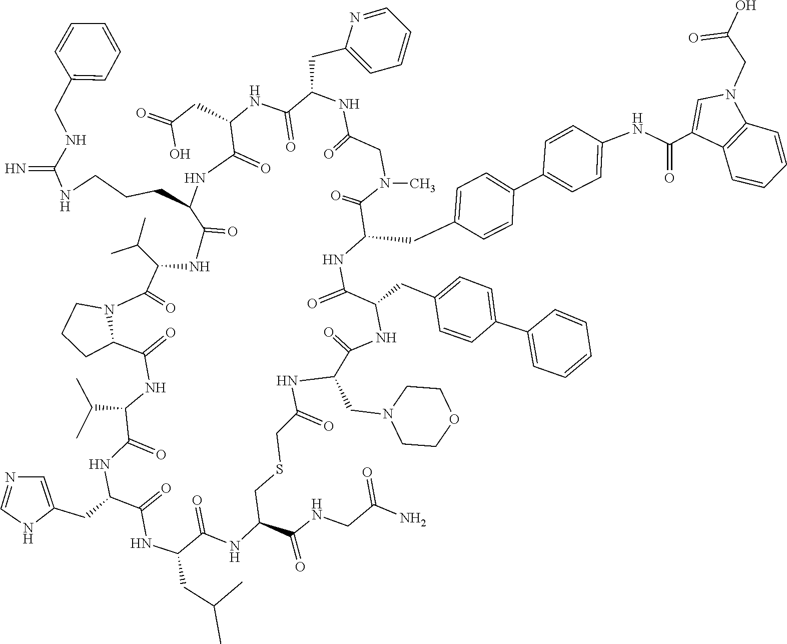 [SMILES: CC(C)C[C@@H]1NC(=O)[C@H](Cc2cnc[nH]2)NC(=O)[C@H](C(C)C)NC(=O)[C@@H]2CCCN2C(=O)[C@H](C(C)C)NC(=O)[C@@H](CCCNC(=N)NCc2ccccc2)NC(=O)[C@H](CC(=O)O)NC(=O)[C@H](Cc2ccccn2)NC(=O)CN(C)C(=O)[C@H](Cc2ccc(-c3ccc(NC(=O)c4cn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 629 was prepared on a 50 Preparative LC/MS. The yield of the product was 6.4 mg, and its estimated purity by LCMS analysis was 91.7%. Analysis condition 1: Retention time=1.56 min. ESI-MS(+) m/z [M+2H]²⁺: 1082.

### Preparation of Example 630

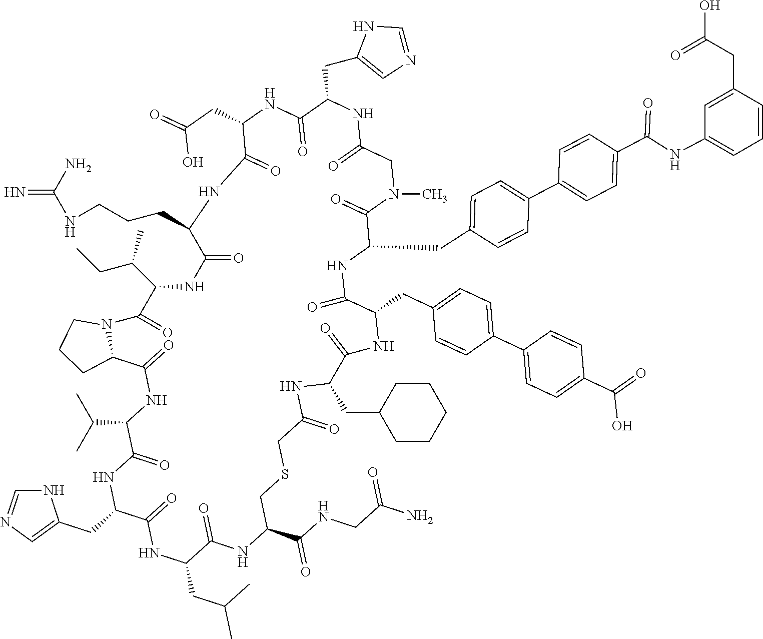 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(C(=O)Nc4cccc(CC(=O)O)c4)cc3)cc2)NC(=O)[C@H](Cc2ccc(-c3ccc(C(=O)O)cc3)cc2)NC(=O)[C@H](CC2CCCCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 630 was prepared on a 50 umol scale. It was purified by Preparative LC/MS. The yield of the product was 12.8 mg, and its estimated purity by LCMS analysis was 99.1%. Analysis condition 2: Retention time=1.26 min. ESI-MS(+) m/z [M+2H]²⁺: 1039.4.

### Preparation of Example 631

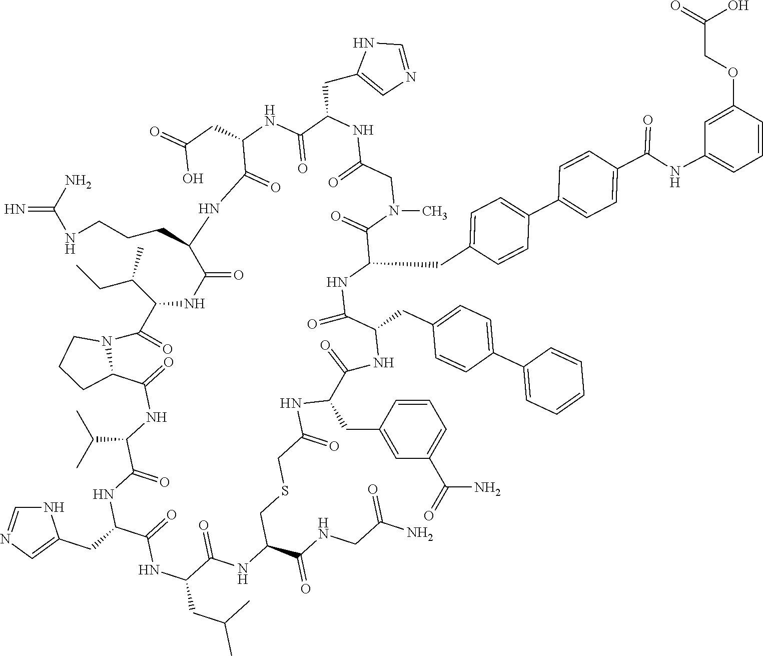 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(C(=O)Nc4cccc(OCC(=O)O)c4)cc3)cc2)NC(=O)[C@H](Cc2ccc(-c3ccccc3)cc2)NC(=O)[C@H](Cc2cccc(C(N)=O)c2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 631 was prepared on a 50 umol scale. It was purified by Preparative LC/MS. The yield of the product was 7.4 mg, and its estimated purity by LCMS analysis was 99.2%. Analysis condition 2: Retention time=1.3 min. ESI-MS(+) m/z [M+3H]³⁺: 696.1.

### Preparation of Example 632

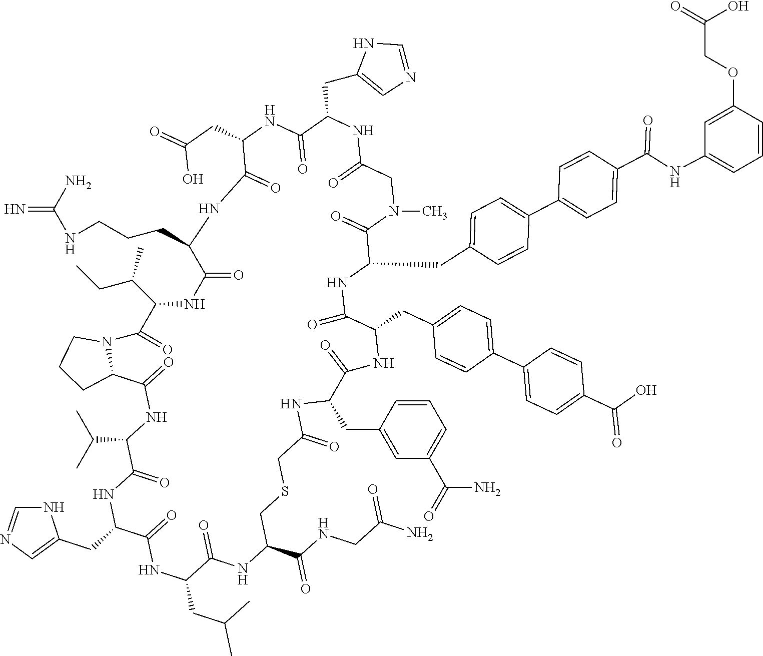 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(C(=O)Nc4cccc(OCC(=O)O)c4)cc3)cc2)NC(=O)[C@H](Cc2ccc(-c3ccc(C(=O)O)cc3)cc2)NC(=O)[C@H](Cc2cccc(C(N)=O)c2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 632 was prepared on a 50 umol scale. It was purified by Preparative LC/MS. The yield of the product was 2.7 mg, and its estimated purity by LCMS analysis was 100%. Analysis condition 1: Retention time=0.96 min. ESI-MS(+) m/z [M+2H]²⁺: 1065.9.

### Preparation of Example 633

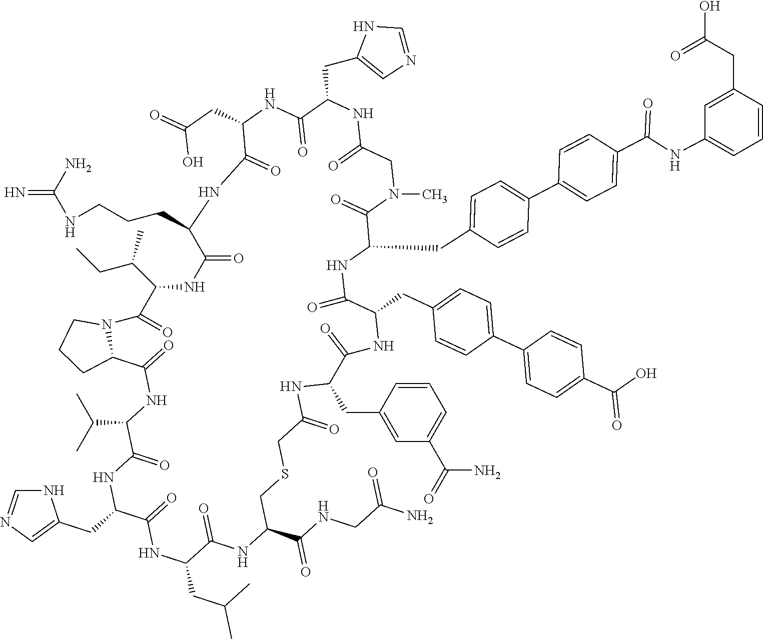 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(C(=O)Nc4cccc(CC(=O)O)c4)cc3)cc2)NC(=O)[C@H](Cc2ccc(-c3ccc(C(=O)O)cc3)cc2)NC(=O)[C@H](Cc2cccc(C(N)=O)c2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 633 was prepared on a 50 umol scale. It was purified by Preparative LC/MS. The yield of the product was 10.6 mg, and its estimated purity by LCMS analysis was 90.8%. Analysis condition 1: Retention time=1.21 min. ESI-MS(+) m/z [M+2H]²⁺: 1036.1.

### Preparation of Example 634

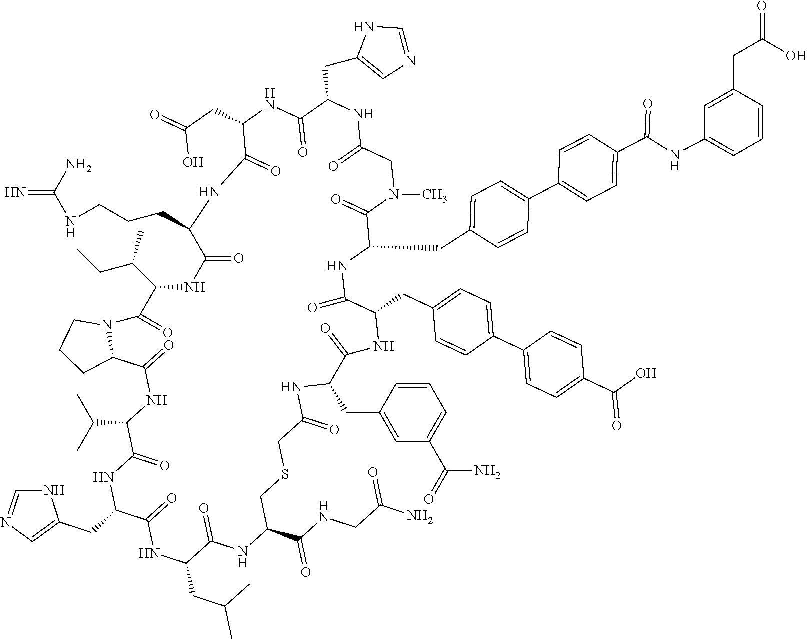 [SMILES: CC[C@H](C)[C@@H]1NC(=O)[C@@H](CCCNC(=N)N)NC(=O)[C@H](CC(=O)O)NC(=O)[C@H](Cc2cnc[nH]2)NC(=O)CN(C)C(=O)[C@H](Cc2ccc(-c3ccc(C(=O)Nc4cccc(CC(=O)O)c4)cc3)cc2)NC(=O)[C@H](Cc2ccc(-c3ccc(C(=O)O)cc3)cc2)NC(=O)[C@H](Cc2cccc(C(N)=O)c2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 634 was prepared on a 50 umol scale. It was purified by Preparative LC/MS. The yield of the product was 6.7 mg, and its estimated purity by LCMS analysis was 95.9%. Analysis condition 2: Retention time=1.13 min. ESI-MS(+) m/z [M+2H]²⁺: 1057.6.

### Preparation of Example 635

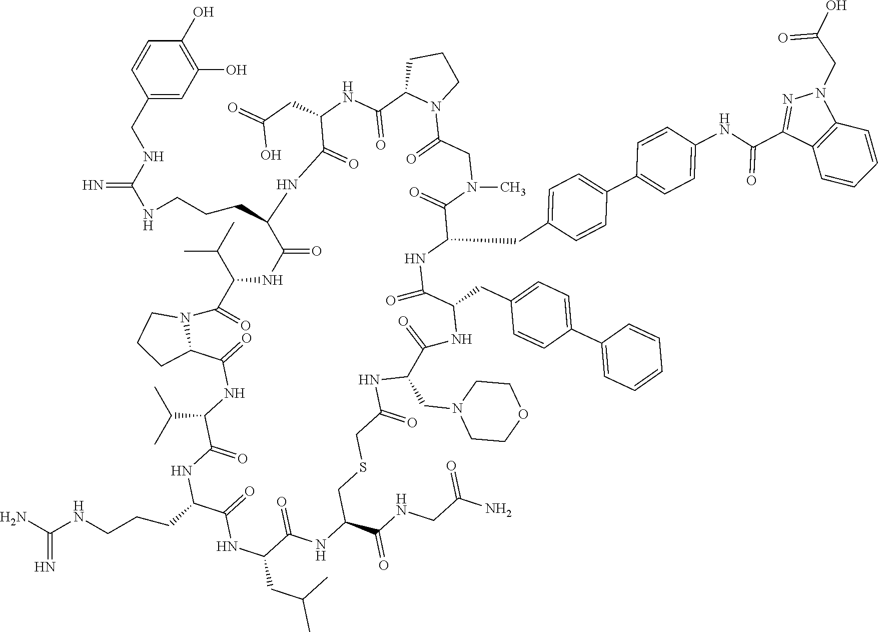 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2ccc(O)c(O)c2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 635 was prepared on a 50 umol scale. It was purified by Preparative LC/MS. The yield of the product was 11 mg, and its estimated purity by LCMS analysis was 96.2%. Analysis condition 1: Retention time=1.31 min. ESI-MS(+) m/z [M+2H]²⁺: 1082.4.

### Preparation of Example 636

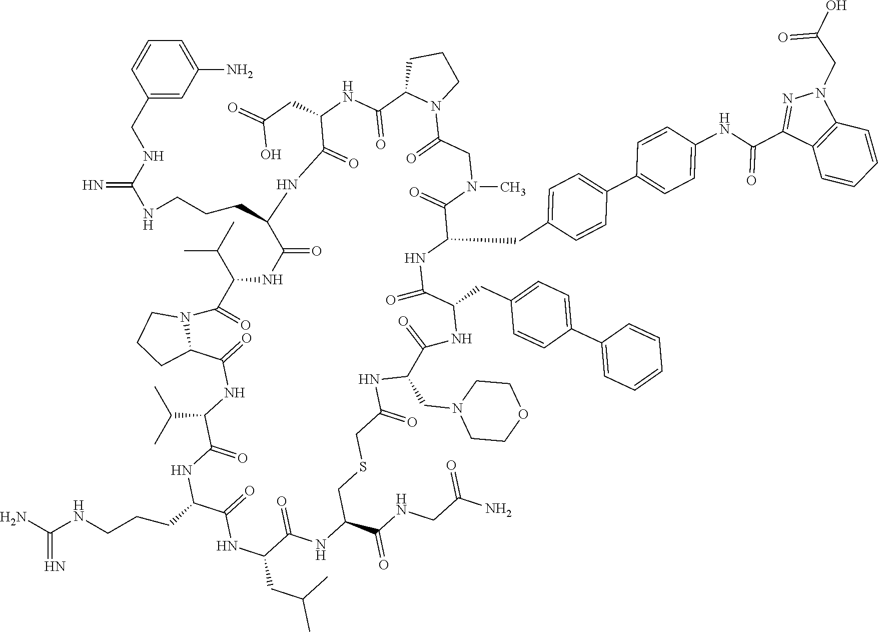 [SMILES: CC(C)C[C@@H]1NC(=O)[C@H](CCCNC(=N)N)NC(=O)[C@H](C(C)C)NC(=O)[C@@H]2CCCN2C(=O)[C@H](C(C)C)NC(=O)[C@@H](CCCNC(=N)NCc2cccc(N)c2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 636 was prepared on a 50 umol scale. It was purified by Preparative LC/MS. The yield of the product was 8.2 mg, and its estimated purity by LCMS analysis was 93.6%. Analysis condition 1: Retention time=1.34 min. ESI-MS(+) m/z [M+2H]²⁺: 1074.2.

### Preparation of Example 637

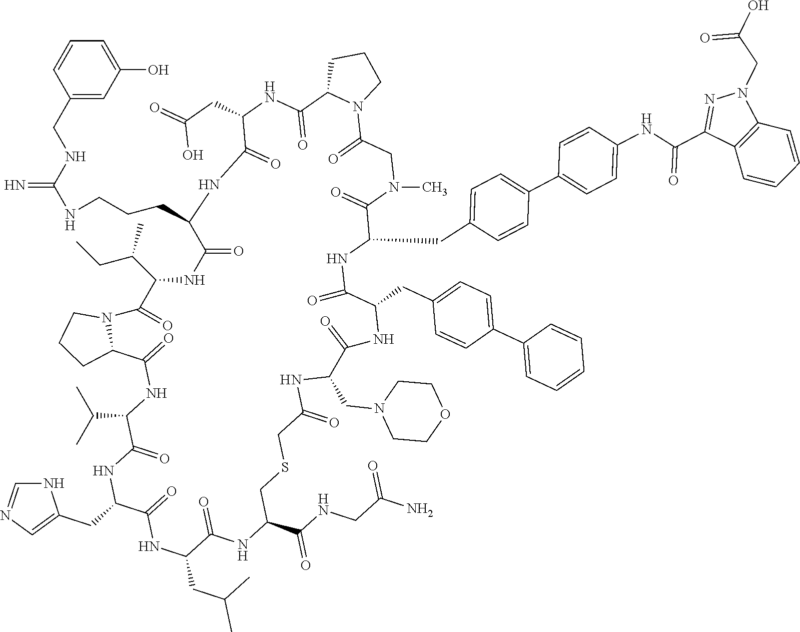 [SMILES: CC[C@H](C)[C@@H]1NC(=O)[C@@H](CCCNC(=N)NCc2cccc(O)c2)NC(=O)[C@H](CC(=O)O)NC(=O)[C@@H]2CCCN2C(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC(=O)[C@H](CC(C)C)NC(=O)[C@H](Cc2cnc[nH]2)NC(=O)[C@H](C(C)C)NC(=O)[C@@H]2CCCN2C1=O]

Example 637 was prepared on a 100 umol scale. It was purified by Preparative LC/MS. The yield of the product was 27.2 mg, and its estimated purity by LCMS analysis was 100%. Analysis condition 1: Retention time=1.4 min. ESI-MS(+) m/z [M+2H]²⁺: 1071.9.

### Preparation of Example 638

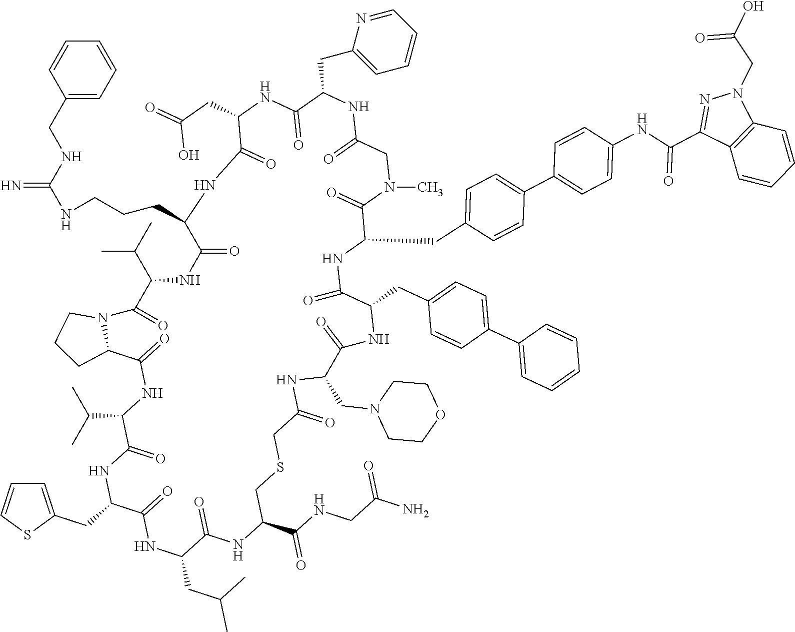 [SMILES: CC(C)C[C@@H]1NC(=O)[C@H](Cc2cccs2)NC(=O)[C@H](C(C)C)NC(=O)[C@@H]2CCCN2C(=O)[C@H](C(C)C)NC(=O)[C@@H](CCCNC(=N)NCc2ccccc2)NC(=O)[C@H](CC(=O)O)NC(=O)[C@H](Cc2ccccn2)NC(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 638 was prepared on a 50 umol scale. It was purified by Preparative LC/MS. The yield of the product was 13 mg, and its estimated purity by LCMS analysis was 94.3%. Analysis condition 1: Retention time=1.58 min. ESI-MS(+) m/z [M+2H]²⁺: 1090.5.

### Preparation of Example 639

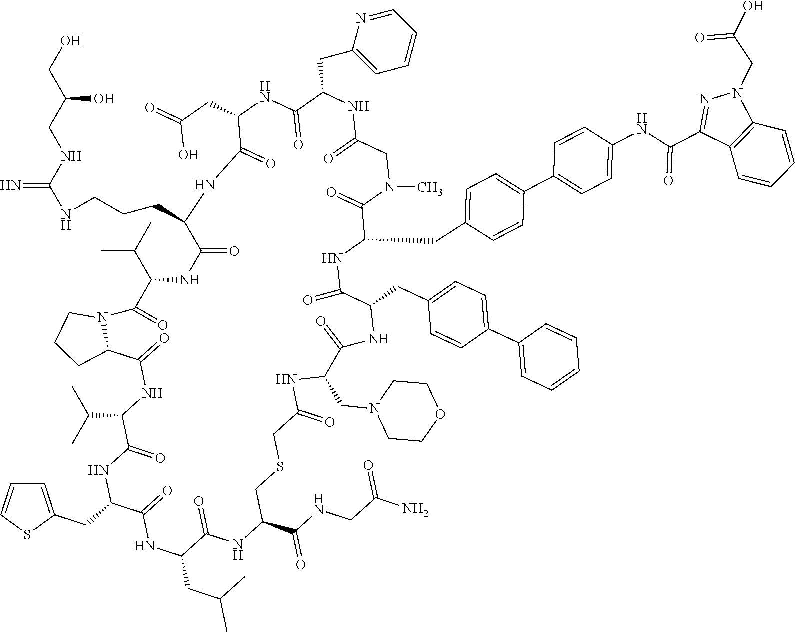 [SMILES: CC(C)C[C@@H]1NC(=O)[C@H](Cc2cccs2)NC(=O)[C@H](C(C)C)NC(=O)[C@@H]2CCCN2C(=O)[C@H](C(C)C)NC(=O)[C@@H](CCCNC(=N)NC[C@H](O)CO)NC(=O)[C@H](CC(=O)O)NC(=O)[C@H](Cc2ccccn2)NC(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 639 was prepared on a 50 umol scale. It was purified by Preparative LC/MS. The yield of the product was 33.9 mg, and its estimated purity by LCMS analysis was 100%. Analysis condition 1: Retention time=1.45 min. ESI-MS(+) m/z [M+2H]²⁺: 1082.6.

### Preparation of Example 640

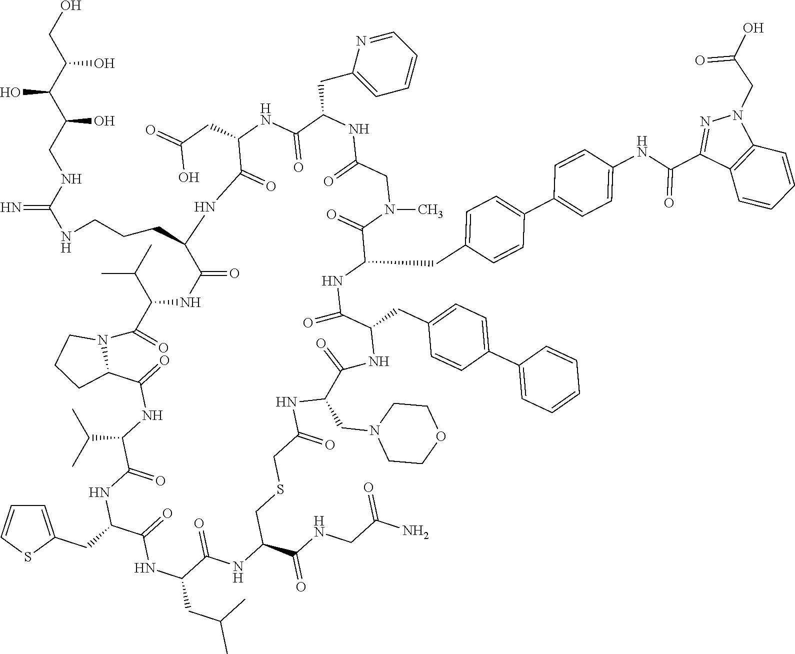 [SMILES: CC(C)C[C@@H]1NC(=O)[C@H](Cc2cccs2)NC(=O)[C@H](C(C)C)NC(=O)[C@@H]2CCCN2C(=O)[C@H](C(C)C)NC(=O)[C@@H](CCCNC(=N)NC[C@H](O)[C@@H](O)[C@@H](O)CO)NC(=O)[C@H](CC(=O)O)NC(=O)[C@H](Cc2ccccn2)NC(=O)CN(C)C(=O)[C@H](Cc2ccc(-c3ccc(NC(=O)c4nn(CC(=O)O)c5ccccc45)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 640 was prepared on a 50 umol scale. It was purified by Preparative LC/MS. The yield of the product was 16.4 mg, and its estimated purity by LCMS analysis was 89.1%. Analysis condition 1: Retention time=1.44 min. ESI-MS(+) m/z [M+2H]²⁺: 1112.5.

### Preparation of Example 641

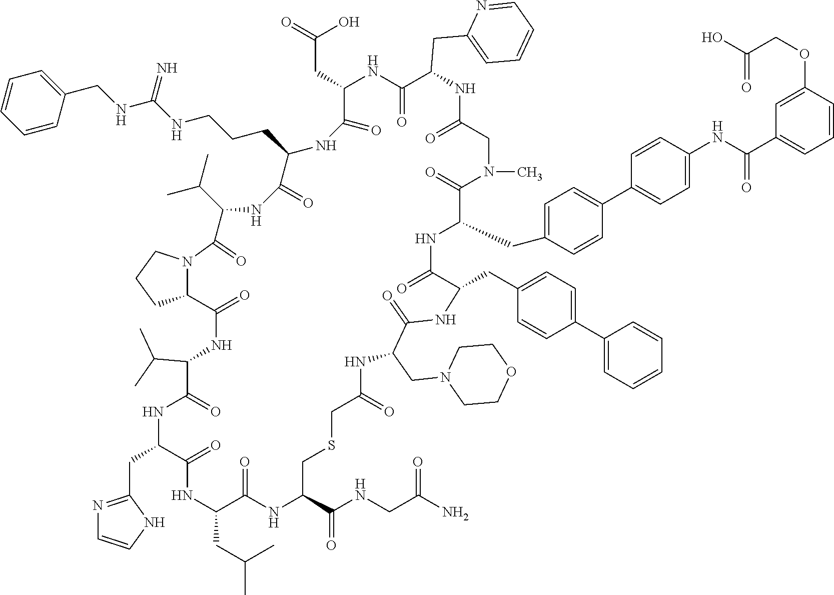 [SMILES: CC(C)C[C@@H]1NC(=O)[C@H](Cc2ncc[nH]2)NC(=O)[C@H](C(C)C)NC(=O)[C@@H]2CCCN2C(=O)[C@H](C(C)C)NC(=O)[C@@H](CCCNC(=N)NCc2ccccc2)NC(=O)[C@H](CC(=O)O)NC(=O)[C@H](Cc2ccccn2)NC(=O)CN(C)C(=O)[C@H](Cc2ccc(-c3ccc(NC(=O)c4cccc(OCC(=O)O)c4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 641 was prepared on a 50 Preparative LC/MS. The yield of the product was 1.6 mg, and its estimated purity by LCMS analysis was 94.6%. Analysis condition 1: Retention time=1.45 min. ESI-MS(+) m/z [M+2H]²⁺: 1070.5.

### Preparation of Example 642

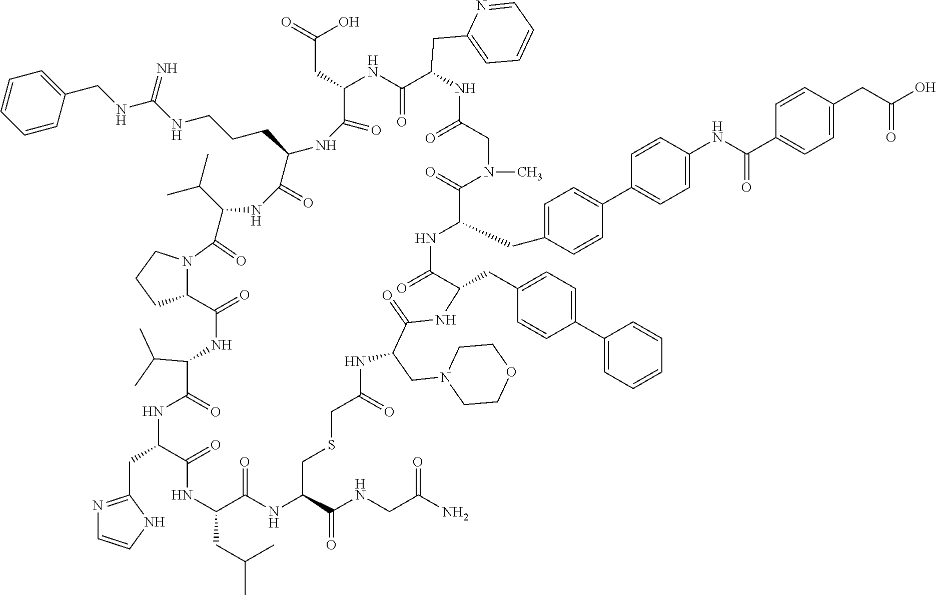 [SMILES: CC(C)C[C@@H]1NC(=O)[C@H](Cc2ncc[nH]2)NC(=O)[C@H](C(C)C)NC(=O)[C@@H]2CCCN2C(=O)[C@H](C(C)C)NC(=O)[C@@H](CCCNC(=N)NCc2ccccc2)NC(=O)[C@H](CC(=O)O)NC(=O)[C@H](Cc2ccccn2)NC(=O)CN(C)C(=O)[C@H](Cc2ccc(-c3ccc(NC(=O)c4ccc(CC(=O)O)cc4)cc3)cc2)NC(=O)[C@H](Cc2ccc(-c3ccccc3)cc2)NC(=O)[C@H](CN2CCOCC2)NC(=O)CSC[C@@H](C(=O)NCC(N)=O)NC1=O]

Example 642 was prepared on a 50 umol scale. It was purified by Preparative LC/MS. The yield of the product was 5.2 mg, and its estimated purity by LCMS analysis was 100%. Analysis condition 1: Retention time=1.45 min. ESI-MS(+) m/z [M+2H]²⁺: 1062.4.

### BIOLOGICAL ASSAYS

The pharmacological properties of the compounds of this invention may be confirmed by a number of biological assays. The exemplified biological assays, which follow, have been carried out with compounds of the invention.

### TNF-Induced HEK-Blue Assay

Test compounds serially diluted in DMSO were plated in an assay plate (LABCYTE, Cat. #LP-0200) at final concentrations ranging from 0.004 UM to 25 μM. TNFα (final concentration 0.5 ng/ml) or CD40L (final concentration 30 ng/ml) in assay buffer [DMEM, 4.5 g/l glucose (Gibco, Cat. 21063-029), 10% FBS (Sigma, F4135), 1% Penicillin-Streptomycin (Gibco, Cat. 15140-122), 1% Anti-Anti (Gibco, Cat. 15240-112) and 2 mM L-glutamine (Gibco, Cat. 25030-081)] was then added to the assay plate. After a 30 minute pre-incubation at 37° C. and 5% CO₂, HEK-Blue™-CD40L cells (INVIVOGEN, Cat. Code hkb-cd40) containing a NF-kB-driven secreted alkaline phosphatase reporter gene were seeded into the assay plate at a density of 20,000 cells per well. This plate was then incubated for 18 h at 37° C. and 5% CO₂. Secreted alkaline phosphatase expression was measured using QUANTI-Blue™ (INVIVOGEN, Cat. Code rep-qb1) according to manufacturer's specifications and the assay plate was read on a PerkinElmer Envision at 620 nm.

Inhibition data for the test compound over a range of concentrations was plotted as percentage inhibition of the test compound (100%=maximum inhibition). IC₅₀ values were determined after correcting for background [(sample read-mean of low control)/(mean of high control-mean of low control)] where by the low control is DMSO without stimulation and high control is DMSO with stimulation. The IC₅₀ is defined as the concentration of test compound which produces 50% inhibition and was quantified using the 4 parameter logistic equation to fit the data. “A” represents an IC₅₀ less than 50 nM, “B” represents an IC₅₀ 50 nM-200 nM, and “C” represents an IC₅₀ from 200 nM to 2 uM

Table 1 lists the IC₅₀ values measured in the TNF induced HEK-Blue assay for Examples 1 to 642 of this invention. The compounds of the present invention, as exemplified by Examples 1 to 642 showed IC₅₀ values measured in the TNF induced HEK-Blue assay of 25 μM or less.

TABLE 1

| | TNF induced HEK- |
|---|---|
| Ex. # | Blue assay IC50 value |
| 1 | C |
| 2 | C |
| 3 | C |
| 4 | C |
| 5 | C |
| 6 | C |
| 7 | C |
| 8 | C |
| 9 | C |
| 10 | C |
| 11 | C |
| 12 | C |
| 13 | C |
| 14 | C |
| 15 | C |
| 16 | C |
| 17 | C |
| 18 | C |
| 19 | C |
| 20 | C |
| 21 | C |
| 22 | C |
| 23 | C |
| 24 | C |
| 25 | C |
| 26 | C |
| 27 | B |
| 28 | C |
| 29 | C |
| 30 | C |
| 31 | C |
| 32 | B |
| 33 | C |
| 34 | C |
| 35 | C |
| 36 | C |
| 37 | C |
| 38 | C |
| 39 | C |
| 40 | B |
| 41 | C |
| 42 | C |
| 43 | C |
| 44 | C |
| 45 | C |
| 46 | C |
| 47 | B |
| 48 | C |
| 49 | B |
| 50 | B |
| 51 | C |
| 52 | C |
| 53 | C |
| 54 | B |
| 55 | B |
| 56 | B |
| 57 | B |
| 58 | B |
| 59 | C |
| 60 | C |
| 61 | C |
| 62 | B |
| 63 | C |
| 64 | C |
| 65 | B |
| 66 | C |
| 67 | C |
| 68 | A |
| 69 | A |
| 70 | C |
| 71 | B |
| 72 | B |
| 73 | C |
| 74 | C |
| 75 | C |
| 76 | A |
| 77 | C |
| 78 | B |
| 79 | A |
| 80 | C |
| 81 | B |
| 82 | C |
| 83 | B |
| 84 | B |
| 85 | C |
| 86 | B |
| 87 | B |
| 88 | C |
| 89 | B |
| 90 | B |
| 91 | C |
| 92 | C |
| 93 | B |
| 94 | B |
| 95 | C |
| 96 | B |
| 97 | B |
| 98 | B |
| 99 | C |
| 100 | C |
| 101 | B |
| 102 | C |
| 103 | C |
| 104 | C |
| 105 | C |
| 106 | B |
| 107 | B |
| 108 | B |
| 109 | C |
| 110 | B |
| 111 | C |
| 112 | C |
| 113 | C |
| 114 | C |
| 115 | C |
| 116 | B |
| 117 | C |
| 118 | C |
| 119 | C |
| 120 | B |
| 121 | C |
| 122 | C |
| 123 | B |
| 124 | B |
| 125 | A |
| 126 | B |
| 127 | B |
| 128 | A |
| 129 | C |
| 130 | C |
| 131 | B |
| 132 | B |
| 133 | C |
| 134 | B |
| 135 | C |
| 136 | B |
| 137 | C |
| 138 | C |
| 139 | C |
| 140 | C |
| 141 | B |
| 142 | C |
| 143 | A |
| 144 | A |
| 145 | A |
| 146 | A |
| 147 | A |
| 148 | A |
| 149 | B |
| 150 | B |
| 151 | B |
| 152 | B |
| 153 | A |
| 154 | B |
| 155 | A |
| 156 | B |
| 157 | C |
| 158 | C |
| 159 | A |
| 160 | B |
| 161 | B |
| 162 | B |
| 163 | C |
| 164 | A |
| 165 | A |
| 166 | A |
| 167 | B |
| 168 | A |
| 169 | A |
| 170 | A |
| 171 | A |
| 172 | A |
| 173 | B |
| 174 | B |
| 175 | A |
| 176 | A |
| 177 | B |
| 178 | A |
| 179 | B |
| 180 | B |
| 181 | A |
| 182 | B |
| 183 | B |
| 184 | A |
| 185 | A |
| 186 | B |
| 187 | B |
| 188 | A |
| 189 | C |
| 190 | B |
| 191 | B |
| 192 | B |
| 193 | B |
| 194 | B |
| 195 | A |
| 196 | A |
| 197 | B |
| 198 | A |
| 199 | A |
| 200 | B |
| 201 | A |
| 202 | C |
| 203 | A |
| 204 | B |
| 205 | B |
| 206 | A |
| 207 | C |
| 208 | B |
| 209 | C |
| 210 | A |
| 211 | C |
| 212 | A |
| 213 | A |
| 214 | B |
| 215 | B |
| 216 | C |
| 217 | B |
| 218 | B |
| 219 | B |
| 220 | A |
| 221 | A |
| 222 | A |
| 223 | A |
| 224 | B |
| 225 | B |
| 226 | B |
| 227 | B |
| 228 | A |
| 229 | B |
| 230 | B |
| 231 | B |
| 232 | A |
| 233 | A |
| 234 | A |
| 235 | C |
| 236 | C |
| 237 | B |
| 238 | A |
| 239 | B |
| 240 | B |
| 241 | A |
| 242 | A |
| 243 | B |
| 244 | C |
| 245 | C |
| 246 | B |
| 247 | B |
| 248 | B |
| 249 | B |
| 250 | B |
| 251 | A |
| 252 | A |
| 253 | B |
| 254 | C |
| 255 | B |
| 256 | B |
| 257 | A |
| 258 | B |
| 259 | A |
| 260 | A |
| 261 | A |
| 262 | B |
| 263 | B |
| 264 | B |
| 265 | B |
| 266 | B |
| 267 | B |
| 268 | A |
| 269 | B |
| 270 | B |
| 271 | B |
| 272 | B |
| 273 | A |
| 274 | A |
| 275 | B |
| 276 | A |
| 277 | A |
| 278 | B |
| 279 | B |
| 280 | B |
| 281 | B |
| 282 | B |
| 283 | A |
| 284 | B |
| 285 | A |
| 286 | B |
| 287 | A |
| 288 | C |
| 289 | B |
| 290 | B |
| 291 | A |
| 292 | A |
| 293 | A |
| 294 | A |
| 295 | A |
| 296 | A |
| 297 | A |
| 298 | B |
| 299 | B |
| 300 | B |
| 301 | B |
| 302 | B |
| 303 | C |
| 304 | A |
| 305 | A |
| 306 | B |
| 307 | B |
| 308 | A |
| 309 | B |
| 310 | B |
| 311 | B |
| 312 | C |
| 313 | C |
| 314 | C |
| 315 | A |
| 316 | B |
| 317 | B |
| 318 | B |
| 319 | B |
| 320 | C |
| 321 | B |
| 322 | B |
| 323 | B |
| 324 | B |
| 325 | C |
| 326 | B |
| 327 | B |
| 328 | B |
| 329 | A |
| 330 | B |
| 331 | A |
| 332 | C |
| 333 | A |
| 334 | A |
| 335 | B |
| 336 | A |
| 337 | B |
| 338 | A |
| 339 | A |
| 340 | A |
| 341 | A |
| 342 | A |
| 343 | A |
| 344 | A |
| 345 | A |
| 346 | A |
| 347 | A |
| 348 | B |
| 349 | C |
| 350 | A |
| 351 | A |
| 352 | A |
| 353 | A |
| 354 | B |
| 355 | A |
| 356 | A |
| 357 | B |
| 358 | A |
| 359 | A |
| 360 | A |
| 361 | A |
| 362 | A |
| 363 | A |
| 364 | A |
| 365 | A |
| 366 | A |
| 367 | A |
| 368 | A |
| 369 | B |
| 370 | A |
| 371 | A |
| 372 | A |
| 373 | A |
| 374 | A |
| 375 | A |
| 376 | B |
| 377 | B |
| 378 | A |
| 379 | B |
| 380 | B |
| 381 | B |
| 382 | A |
| 383 | B |
| 384 | B |
| 385 | A |
| 386 | B |
| 387 | A |
| 388 | A |
| 389 | A |
| 390 | A |
| 391 | B |
| 392 | B |
| 393 | A |
| 394 | B |
| 395 | A |
| 396 | A |
| 397 | B |
| 398 | A |
| 399 | C |
| 400 | C |
| 401 | B |
| 402 | B |
| 403 | B |
| 404 | B |
| 405 | B |
| 406 | B |
| 407 | B |
| 408 | A |
| 409 | B |
| 410 | C |
| 411 | B |
| 412 | B |
| 413 | C |
| 414 | B |
| 415 | B |
| 416 | B |
| 417 | A |
| 418 | B |
| 419 | C |
| 420 | B |
| 421 | B |
| 422 | A |
| 423 | A |
| 424 | B |
| 425 | A |
| 426 | A |
| 427 | B |
| 428 | B |
| 429 | C |
| 430 | A |
| 431 | B |
| 432 | B |
| 433 | B |
| 434 | B |
| 435 | A |
| 436 | A |
| 437 | C |
| 438 | B |
| 439 | B |
| 440 | A |
| 441 | A |
| 442 | B |
| 443 | A |
| 444 | A |
| 445 | A |
| 446 | B |
| 447 | B |
| 448 | A |
| 449 | A |
| 450 | A |
| 451 | A |
| 452 | B |
| 453 | A |
| 454 | A |
| 455 | B |
| 456 | B |
| 457 | A |
| 458 | A |
| 459 | B |
| 460 | A |
| 461 | A |
| 462 | A |
| 463 | B |
| 464 | A |
| 465 | A |
| 466 | A |
| 467 | A |
| 468 | A |
| 469 | A |
| 470 | A |
| 471 | A |
| 472 | B |
| 473 | A |
| 474 | B |
| 475 | A |
| 476 | A |
| 477 | A |
| 478 | B |
| 479 | A |
| 480 | B |
| 481 | A |
| 482 | A |
| 483 | A |
| 484 | A |
| 485 | A |
| 486 | A |
| 487 | A |
| 488 | A |
| 489 | A |
| 490 | B |
| 491 | B |
| 492 | A |
| 493 | B |
| 494 | A |
| 495 | A |
| 496 | A |
| 497 | A |
| 498 | B |
| 499 | A |
| 500 | A |
| 501 | B |
| 502 | A |
| 503 | A |
| 504 | A |
| 505 | B |
| 506 | A |
| 507 | A |
| 508 | A |
| 509 | A |
| 510 | A |
| 511 | B |
| 512 | A |
| 513 | A |
| 514 | A |
| 515 | A |
| 516 | A |
| 517 | A |
| 518 | B |
| 519 | B |
| 520 | A |
| 521 | A |
| 522 | A |
| 523 | A |
| 524 | B |
| 525 | A |
| 526 | A |
| 527 | A |
| 528 | A |
| 529 | B |
| 530 | A |
| 531 | A |
| 532 | A |
| 533 | A |
| 534 | A |
| 535 | A |
| 536 | A |
| 537 | B |
| 538 | C |
| 539 | A |
| 540 | A |
| 541 | A |
| 542 | A |
| 543 | C |
| 544 | C |
| 545 | B |
| 546 | A |
| 547 | A |
| 548 | B |
| 549 | B |
| 550 | B |
| 551 | A |
| 552 | B |
| 553 | B |
| 554 | A |
| 555 | A |
| 556 | A |
| 557 | A |
| 558 | A |
| 559 | A |
| 560 | A |
| 561 | A |
| 562 | B |
| 563 | A |
| 564 | A |
| 565 | A |
| 566 | A |
| 567 | A |
| 568 | A |
| 569 | A |
| 570 | B |
| 571 | B |
| 572 | A |
| 573 | B |
| 574 | A |
| 575 | B |
| 576 | B |
| 577 | B |
| 578 | A |
| 579 | A |
| 580 | A |
| 581 | B |
| 582 | A |
| 583 | B |
| 584 | B |
| 585 | B |
| 586 | A |
| 587 | A |
| 588 | A |
| 589 | A |
| 590 | A |
| 591 | A |
| 592 | A |
| 593 | A |
| 594 | A |
| 595 | A |
| 596 | B |
| 597 | A |
| 598 | A |
| 599 | A |
| 600 | A |
| 601 | A |
| 602 | A |
| 603 | A |
| 604 | A |
| 605 | C |
| 606 | A |
| 607 | A |
| 608 | A |
| 609 | A |
| 610 | A |
| 611 | A |
| 612 | B |
| 613 | B |
| 614 | B |
| 615 | B |
| 616 | B |
| 617 | A |
| 618 | B |
| 619 | A |
| 620 | A |
| 621 | A |
| 622 | A |
| 623 | A |
| 624 | A |
| 625 | A |
| 626 | B |
| 627 | B |
| 628 | A |
| 629 | B |
| 630 | B |
| 631 | B |
| 632 | B |
| 633 | B |
| 634 | B |
| 635 | A |
| 636 | A |
| 637 | A |
| 638 | A |
| 639 | A |
| 640 | B |
| 641 | B |
| 642 | C |

1. A compound selected from any of the exemplified compounds.

2. A pharmaceutical composition comprising one or more compounds according to claim 1 or a salt thereof; and a pharmaceutically acceptable carrier or diluent.

3. A method for treating a disease comprising administering a compound of claim 1, wherein the disease is selected from Crohn's disease, ulcerative colitis, asthma, graft versus host disease, allograft rejection, chronic obstructive pulmonary disease, Graves' disease, rheumatoid arthritis, systemic lupus erythematosus, lupus nephritis, cutaneous lupus, psoriasis, cryopyrin-associated periodic syndromes, TNF receptor associated periodic syndrome, familial Mediterranean fever, adult onset stills, systemic onset juvenile idiopathic arthritis, multiple sclerosis, neuropathic pain, gout, and gouty arthritis.

4. A method for treating a disease comprising administering a compound of claim 2, wherein the disease is selected from Crohn's disease, ulcerative colitis, asthma, graft versus host disease, allograft rejection, chronic obstructive pulmonary disease, Graves' disease, rheumatoid arthritis, systemic lupus erythematosus, lupus nephritis, cutaneous lupus, psoriasis, cryopyrin-associated periodic syndromes, TNF receptor associated periodic syndrome, familial Mediterranean fever, adult onset stills, systemic onset juvenile idiopathic arthritis, multiple sclerosis, neuropathic pain, gout, and gouty arthritis.
